(19) United States
(12) Patent Application Publication
(10) Pub. No.: US 2026/0297165 A1
(43) Pub. Date: Oct. 1, 2026

(54) COMPOSITIONS AND METHODS FOR EXPRESSING THERAPEUTICS

(71) Applicant: Avirmax Biopharma Inc., Hayward, CA (US)

(72) Inventors: Shengjiang LIU, Lafayette, CA (US); Jianwu CHEN, Danville, CA (US)

(21) Appl. No.: 19/531,546

(22) Filed: Feb. 5, 2026

Related U.S. Application Data
(63) Continuation of application No. PCT/US2024/041100, filed on Aug. 6, 2024.
(60) Provisional application No. 63/531,202, filed on Aug. 7, 2023.
(60) Provisional application No. 63/665,105, filed on Jun. 27, 2024.

(51) Int. Cl. C07K 14/705; A61K 38/17; A61K 38/22; A61K 48/00; C07K 14/575; C12N 15/86

(52) U.S. Cl. CPC C07K 14/70596; A61K 38/177; A61K 38/22; A61K 48/0033; A61K 48/005; C07K 14/575; C12N 15/86; C07K 2319/30; C12N 2750/14143

(57) ABSTRACT

Described herein are compositions for modulating transgene expression of one or more therapeutics. Also described herein are methods for using the compositions described herein for modulating transgene expression and for treating a disease or condition.

### CROSS-REFERENCE

This application is a continuation of International Application No. PCT/US2024/041100, filed Aug. 6, 2024, which claims the benefit of U.S. Provisional Application Ser. No. 63/531,202 filed on Aug. 7, 2023, and U.S. Provisional Application Ser. No. 63/665,105 filed on Jun. 27, 2024, each of which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 5, 2026, is named 59561-713.301_SL.xml and is 391,284 bytes in size.

### BACKGROUND

Neovascularization, including vasculogenesis, angiogenesis, and arteriogenesis, is regulated by a wide variety of cell signaling pathways. One of the signaling pathways is regulated by vascular endothelium growth factors (VEGFs). VEGFs are strong mitogens for endothelial cells, inducing proliferation, migration, blood vessel tubing formation, and permeability. As such, increase in VEGF signaling transduction pathway increases neovascularization signal, while decrease or inhibition of VEGF signaling transduction pathway decreases neovascularization signal. VEGF inhibition is one of the most popular treatment options for disease or condition related to neovascularization. For example, Treatment of ocular diseases often involves the use of angiogenesis inhibitors such as VEGF inhibitor.

### SUMMARY

Current treatments employing VEGF inhibitors can be cumbersome due to the short half-life of the VEGF inhibitor, which leads to the need for repeated monthly injections for achieving and sustaining suppression of neovascularization. Therefore, there remains a need for a therapeutic for treating ocular diseases. There also remains a need for therapeutics for inhibiting neovascularization by targeting signaling pathways other than or in combination with VEGF signaling pathways. Accordingly, described herein, in some aspects, is an engineered polynucleotide comprising one or more expression cassettes, the one or more expression cassettes encoding a first angiogenesis inhibitor and a second angiogenesis inhibitor. In some embodiments, the first angiogenesis inhibitor and the second angiogenesis inhibitor are covalently connected by a linker. In some embodiments, the first angiogenesis inhibitor comprises a complement inhibitor. In some embodiments, the complement inhibitor comprises a complement 3 inhibitor or a C3 degraded fragment. In some embodiments, the complement 3 inhibitor comprises an amino acid sequence that is at least 80% identical to any one of SEQ ID NOs: 1-15. In some embodiments, the first angiogenesis inhibitor or the second angiogenesis inhibitor comprises an inhibitor of a membrane attack complex (MAC). In some embodiments, the inhibitor of the MAC comprises CD59. In some embodiments, the CD59 comprises an amino acid sequence that is at least 80% identical to any one of SEQ ID NOs: 41-45, 312-319, or 325-329. In some embodiments, the second angiogenesis inhibitor comprises a natriuretic peptide. In some embodiments, the natriuretic peptide comprises a C-type natriuretic peptide (CNP). In some embodiments, the natriuretic peptide is covalently connected to an antibody or fragment thereof. In some embodiments, the antibody or fragment thereof comprises a fragment crystallizable (Fc) region. In some embodiments, the natriuretic peptide comprises an amino acid sequence that is at least 80% identical to any one of SEQ ID NOs: 61-72. In some embodiments, the second angiogenesis inhibitor comprises an endostatin or fragment thereof. In some embodiments, encoding a third angiogenesis inhibitor. In some embodiments, the engineered polynucleotide comprises a viral vector. In some embodiments, the viral vector comprises an AAV vector. In some embodiments, the AAV vector is an AAV2 vector. In some embodiments, the AAV vector encodes an engineered AAV capsid. In some embodiments, the engineered AAV capsid comprises an amino acid sequence of any one of SEQ ID NOs: 161-182 and SEQ ID NOs: 191-210. In some embodiments, the first angiogenesis inhibitor comprises the complement 3 inhibitor, and the second angiogenesis inhibitor comprises a CNP36. In some embodiments, the engineered polynucleotide further encoding a third angiogenesis inhibitor. In some embodiments, the third angiogenesis inhibitor comprises an inhibitor of a membrane attack complex (MAC), and wherein the inhibitor of the MAC comprises a CD59. In some embodiments, the first angiogenesis inhibitor comprises the CD59, and the second angiogenesis inhibitor comprises a complement 3 inhibitor fused to an Fc-CNP36. In some embodiments, the first angiogenesis inhibitor comprises a complement 3 inhibitor, and the second angiogenesis inhibitor comprises an endostatin. In some embodiments, the first angiogenesis inhibitor comprises a complement 3 inhibitor, and the second angiogenesis inhibitor comprises an Fc-CNP36, wherein the engineered polynucleotide further encodes a third angiogenesis inhibitor comprising a CD59. In some embodiments, the first angiogenesis inhibitor comprises a CD59, and the second angiogenesis inhibitor comprises a complement 3 inhibitor, wherein the engineered polynucleotide further encodes a third angiogenesis inhibitor comprising a Fc-CNP36. In some embodiments, the first angiogenesis inhibitor comprises a CD59, and the second angiogenesis inhibitor comprises an endostatin, wherein the engineered polynucleotide further encodes a third angiogenesis inhibitor comprising a complement 3 inhibitor. In some embodiments, the first angiogenesis inhibitor comprises a complement 3 inhibitor, and the second angiogenesis inhibitor comprises an endostatin, wherein the engineered polynucleotide further encodes a third angiogenesis inhibitor comprising a CD59.

Described herein, in some aspects, is an engineered polypeptide comprising a first angiogenesis inhibitor and a second angiogenesis inhibitor. In some embodiments, the first angiogenesis inhibitor and the second angiogenesis inhibitor are covalently connected by a linker. In some embodiments, the first angiogenesis inhibitor comprises a complement inhibitor. In some embodiments, the first angiogenesis inhibitor or the second angiogenesis inhibitor comprises an inhibitor of a membrane attack complex (MAC), and wherein the inhibitor of the MAC comprises CD59. In some embodiments, the second angiogenesis inhibitor comprises a natriuretic peptide. In some embodiments, the second angiogenesis inhibitor comprises an endostatin or fragment thereof. In some embodiments, the engineered polypeptide further encoding a third angiogenesis inhibitor.

Described herein, in some aspects, is a vector comprising an engineered polynucleotide described herein, or an engineered polypeptide described herein. In some embodiments, the vector encodes an AAV capsid, and wherein the AAV capsid comprises an engineered AAV capsid.

Described herein, in some aspects, is a viral particle comprising an engineered polynucleotide disclosed herein, an engineered polypeptide disclosed herein, or a vector disclosed herein. In some embodiments, the viral particle comprises an AAV capsid, and wherein the AAV capsid comprises an engineered AAV capsid.

Described herein, in some aspects, is a cell comprising an engineered polynucleotide disclosed herein, an engineered polypeptide disclosed herein, a vector disclosed herein, or a viral particle disclosed herein.

Described herein, in some aspects, is a composition comprising: a complement 3 inhibitor or a C3 degraded fragment comprising a C3a, C3b, iC3b, C3f, C3c, C3d, C3g, or a combination thereof; and a natriuretic peptide. In some embodiments, the natriuretic peptide comprises a C-type natriuretic peptide (CNP).

Described herein, in some aspects, is a composition comprising: a complement 3 inhibitor or a C3 degraded fragment comprising a C3a, C3b, iC3b, C3f, C3c, C3d, C3g, or a combination thereof; and an inhibitor of a membrane attack complex (MAC). In some embodiments, the inhibitor of the MAC comprises CD59.

Described herein, in some aspects, is a composition comprising a CD59 and a natriuretic peptide. In some embodiments, the natriuretic peptide comprises a C-type natriuretic peptide (CNP).

Described herein, in some aspects, is a pharmaceutical composition comprising an engineered polynucleotide disclosed herein, an engineered polypeptide disclosed herein, a vector disclosed herein, a viral particle disclosed herein, a cell disclosed herein, or a composition disclosed herein.

Described herein, in some aspects, is a method comprising contacting a cell obtained from a subject an engineered polynucleotide disclosed herein, an engineered polypeptide disclosed herein, a vector disclosed herein, a viral particle disclosed herein, a cell disclosed herein, a composition disclosed herein, or a pharmaceutical composition disclosed herein.

Described herein, in some aspects, is a method of treating a disease or condition in a subject, comprising: administering to the subject an engineered polynucleotide disclosed herein, an engineered polypeptide disclosed herein, a vector disclosed herein, a viral particle disclosed herein, a cell disclosed herein, a composition disclosed herein, or a pharmaceutical composition disclosed herein.

Described herein, in some aspects, is a method of treating a disease or condition in a subject, the method comprising administering to the subject an engineered polynucleotide comprising one or more expression cassettes, the one or more expression cassettes encoding a first angiogenesis inhibitor and a second angiogenesis inhibitor.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 illustrates vector construct for expressing a complement C3 inhibitor (C3i).

FIG. 2A and FIG. 2B illustrate vector construct for expressing an C3i operatively coupled to a natriuretic polypeptide.

FIG. 3 illustrates vector construct for expressing an C3i, an CD59, and a natriuretic polypeptide.

FIG. 4 illustrates exemplary vectors for expressing the angiogenesis inhibitors described herein.

FIGS. 5A-5C illustrate AAV constructs created and tested in Example 3.

FIG. 6 illustrates fusion proteins expressed by cells on 6-well plates were detected by SDS-PAGE and Western blot using HRP-conjugated goat anti-human IgG1 Fc antibody. Lane content: 1. Vector GAM 3d; 2. Vector EKQ 3d; 3. Vector CME 3d; 4. Non-transfected 3d; 5. Vector GAM 6d; 6. Vector EKQ 6d; 7. Vector CME 6d; 8. Non-transfected 6d.

FIG. 7 illustrates fusion proteins expressed by the cells in T125 flasks were detected by SDS-PAGE and Western blot using HRP-conjugated goat anti-human IgG1 Fc antibody. Lane content: 1. Vector GAM 3d; 2. Vector EKQ 3d; 3. Vector CME 3d; 4. non-transfected 3d; 5. Vector GAM 6d; 6. Vector EKQ 6d; 7. Vector CME 6d; 8. non-transfected 6d.

FIG. 8 illustrates fusion proteins expressed by the cells on 6-well plates were detected by SDS-PAGE and Western blot using rat anti-human CNP antibodies. Lane content: 1. Vector GAM 3d; 2. Vector EKQ 3d; 3. Vector CME 3d; 4. Non-transfected 3d; 5. Vector GAM 6d; 6. Vector EKQ 6d; 7. Vector CME 6d; 8. Non-transfected 6d.

FIG. 9 illustrates fusion proteins expressed by the cells on T125 flasks were detected by SDS-PAGE and Western blot using rat anti-human CNP antibodies based on Western blot analysis. Lane content: 1. Vector GAM 3d; 2. Vector EKQ 3d; 3. Vector CME 3d; 4. non-transfected 3d; 5. Vector GAM 6d; 6. Vector EKQ 6d; 7. Vector CME 6d; 8. Non-transfected 6d.

FIG. 10 illustrates membrane-bound CD59 was expressed in transfected cells based on Western blot analysis. Lane content: 1. Vector GTM; 2. Vector GTP; 3. Vector GTQ; 4. Vector GTR; 5. Vector GAT; 6. Vector EKQ; 7. Non-transfected; 8. Purified CD59 protein.

FIG. 11 illustrates Soluble CD59 (sCD59) protein in the cell culture supernatant at day 3 was detected using HRP-conjugated anti-human CD59 antibody based on Western blot analysis. Lane content: 1. Vector GCK; 2. Vector GCM; 3. Vector GEM; 4. Vector GEP; 5. Vector EKQ; 6. Non-transfected; 7. Purified CD59 protein.

FIG. 12 illustrates soluble CD59 (sCD59) protein in the cell culture supernatant at day 6 was detected using HRP-conjugated anti-human CD59 antibody based on Western blot analysis. Lane content: 1. Vector GCK; 2. Vector GCM; 3. Vector GEM; 4. Vector GEP; 5. Vector EKQ; 6. Non-transfected; 7. Purified CD59 protein.

FIG. 13 illustrates schematic AAV vector designs for Example 5.

FIG. 14 illustrates soluble CD59 (sCD59) protein large scale production in cell culture supernatant at day 6 was shown to be pure using SDS-PAGE gel analysis.

FIGS. 15A and 15B illustrate graphs depicting the results of a cell lysis inhibition assay. FIG. 15A illustrates a dose-response curve of % cytotoxicity from increasing concentration of normal human serum (NHS). FIG. 15B illustrates a dose-response curve of % max cytotoxicity normalized to BSA control compared to Vector GEM and CP40 cytoprotective effects.

FIG. 16 illustrates schematic AAV vector designs for Example 6.

FIG. 17 illustrates fusion proteins expressed by cells detected by SDS-PAGE and Western blot using HRP-conjugated mouse anti-human Fc antibodies. Lane content: M. Pre-stain protein marker; 1. Vector GAM; 2. Vector GGE; 3. Vector GGG; 4. Vector GGQ; 5. Vector GKA; 6. Vector GKK; 7. Vector EKQ; 8. Vector CPE; 9. Non-transfected cells.

FIG. 18 illustrates fusion proteins expressed by cells detected by SDS-PAGE and Western blot using Biotin-labeled goat-anti-human IgG antibodies and HRP-Streptavidin conjugate. Lane content: M. Pre-stain protein marker; 1. Vector GGE (repeat 1); 2. Vector GGE (repeat 2); 3. Vector GGG (repeat 1); 4. Vector GGG (repeat 2); 5. Vector GKA (repeat 1); 6. Vector GKA (repeat 2); PC, positive control, purified Vector GGE proteins.

FIG. 19 illustrate Vector GGG proteins expressed by the cells HEK293LTV in 6 well plates were shown to be pure using SDS-PAGE gel analysis. Lane content: M. Protein marker; 1. cell culture medium; 2. flow through; 3. Wash 1; 4. Wash 2; 5. Elution sample; 6. Proteins in PBS buffer.

FIG. 20 illustrates Vector GGE proteins expressed by the cells HEK293LTV in 6 well plates were shown to be pure using SDS-PAGE gel analysis. Lane content: M. Protein marker; 1. cell culture medium; 2. flow through; 3. Wash 1; 4. Wash 2; 5. Elution sample; 6. Proteins in PBS buffer.

FIGS. 21A-21C illustrate mutant fusion proteins expressed by cells detected by SDS-PAGE and a hemolysis inhibition assay. FIG. 21A illustrates design of mutant proteins derived from Vector GGG. FIG. 21B illustrates protein expressed by Expi293F cells shown to be of high purity using SDS-PAGE gel analysis. Lane content: M: Protein marker; 1, Vector KTP; 2, Vector KTQ; 3, Vector KTR; 4, Vector KAT; 5, Vector KAA; 6, Vector KAC; 7, Vector KAE; 8, Vector KAG; 9, Vector KAK; 10, Vector KAM; 11, Vector KAP; 12, Vector GGG. FIG. 21C illustrates a graph depicting results of a hemolysis inhibition assay for all mutant proteins.

FIGS. 22A and 22B illustrate fusion proteins expressed by cells detected by SDS-PAGE and results from a hemolysis inhibition assay. FIG. 22A illustrates proteins expressed shown to be pure using SDS-PAGE gel analysis. Lane content: M. Protein marker; 1, Vector GGE; 2, Vector GGG; 3, Vector KAG; 4, Vector KKT; 5, Vector KKA; 6, Vector KPT; 7, Vector KPC. FIG. 22B illustrates a graph depicting results of a hemolysis inhibition assay for all mutant proteins.

FIGS. 23A-23C illustrate graphs depicting results from a binding affinity assay of fusion proteins against C3b, C3c, and C3. FIG. 23A illustrates estimated IC50 values and KD vs C3b of Vector KPT, CP40, Vector KKT, Vector KPC, Vector KKA, Vector GGE, Vector KAG, and Vector GGG. FIG. 23B illustrates estimated IC50 values and KD vs C3c of Vector KPT, CP40, Vector KKT, Vector KPC, Vector KKA, Vector GGE, Vector KAG, and Vector GGG. FIG. 23C illustrates estimated IC50 values and KD vs C3 of Vector KPT, CP40, Vector KKT, Vector KPC, Vector KKA, Vector GGE, Vector KAG, and Vector GGG.

FIG. 24 illustrates schematic AAV vector designs for Vector KMR, Vector KKT, and Vector KPP.

FIGS. 25A-25D illustrates protein expression in ARPE-19 cells secreted into cell medium or remaining within the cell. FIG. 25A illustrates expression of CD59 from Vector KMR, Vector KKT, and Vector KPP. FIG. 25B illustrates expression of C3i from Vector KMR, Vector KKT, and Vector KPP. FIG. 25C illustrates expression of Fc-CNP from Vector KKT. FIG. 25D illustrates expression of Endostatin from Vector KPP.

FIG. 26 illustrates schematic AAV vector designs for Vector KGQ, Vector KGR, Vector KKT, and Vector KKA.

FIGS. 27A-27G illustrate results from AAV in vivo expression in mice eyes and statistical analysis. FIG. 27A illustrates a graph depicting results from the expression of CD59 from Vector KGQ, Vector KGR, Vector KKT, and Vector KKA. FIG. 27B illustrates a graph depicting results from the expression of Fc-CNP from Vector KKT. FIG. 27C illustrates a graph depicting results from the expression of hFc from Vector KGQ, Vector KGR, Vector KKT, and Vector KKA. FIG. 27D illustrates a graph depicting results from the expression of endostatin from Vector KKA. FIG. 27E illustrates a graph depicting results from the expression of CD59 and C3i from Vector KGQ and Vector KGR. FIG. 27F illustrates a graph depicting results from the expression of CD59, C3i, and CNP from Vector KKT. FIG. 27G illustrates a graph depicting results from the expression of CD59, C3i, and endostatin from Vector KKA.

FIGS. 28A-28C illustrate results from a ligand binding assay for C3i fusion proteins. FIG. 28A illustrates results from an ELISA assay for binding C3b and Vector KMR, Vector KKT, and Vector KPP proteins and estimated Kd values. FIG. 28B illustrates results from an ELISA assay for binding C3c and Vector KMR, Vector KKT, and Vector KPP proteins and estimated Kd values.

FIG. 28C illustrates results from an ELISA assay for binding C3 and Vector KMR, Vector KKT, and Vector KPP proteins and estimated Kd values.

FIG. 29 illustrates a graph depicting results from a ligand binding assay for NPR-b and Vector KKT protein, Fc1-CNP36, and Fc4-CNP36.

FIG. 30 illustrates a graph depicting results measuring cGMP production in NIG-ETE cells triggered by Vector CME protein (Fc4-CNP36), Vector KKT protein (C3i-Fc4-CNP36), CNP-22, or Cp40.

FIG. 31 illustrates a graph depicting results from an endostatin ligand receptor assay and estimated Kd values for Vector KPP protein.

FIG. 32 illustrates schematic AAV vector designs for Example 12.

FIG. 33 illustrates results from a ligand binding assay for wildtype and variant sCD59 proteins.

The novel features of the disclosure are set forth with particularity in the appended claims. A better understanding of the features and advantages of the present disclosure will be obtained by reference to the following detailed description that sets forth illustrative embodiments.

### DETAILED DESCRIPTION

### Overview

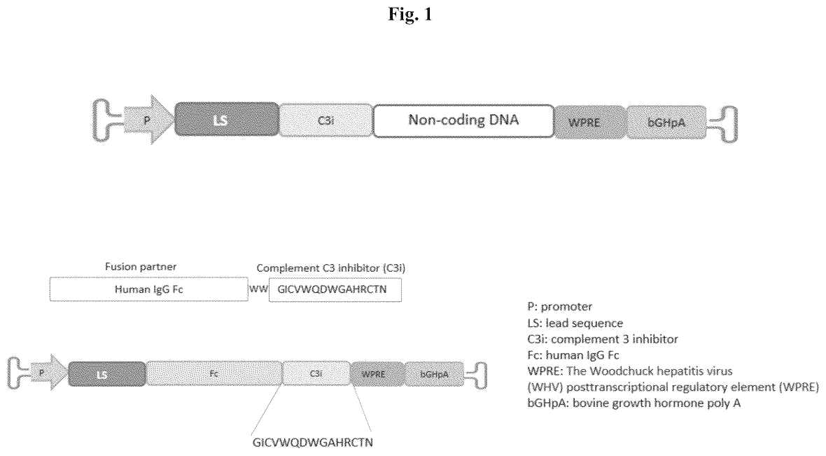

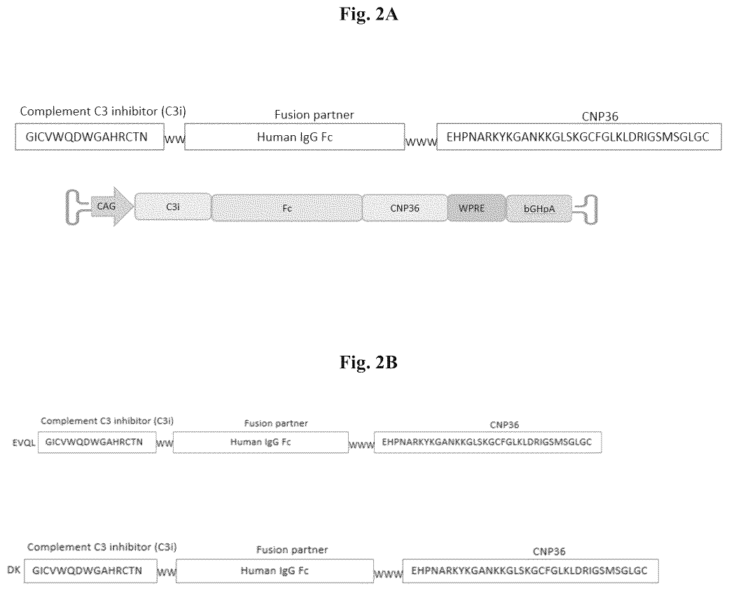

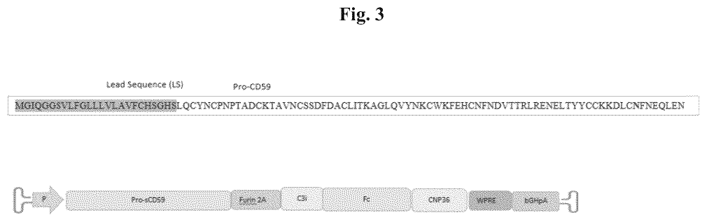

Described herein, in some aspects, is an engineered polynucleotide comprising one or more expression cassettes for encoding one or more angiogenesis inhibitors. In some embodiments, the one or more angiogenesis inhibitors are operatively coupled (e.g., covalently connected). In some embodiments, the one or more angiogenesis inhibitors comprise a complement inhibitor, a natriuretic peptide, an inhibitor of a membrane attack complex (MAC), an VEGF inhibitor, or a combination thereof. In some embodiments, the complement inhibitor comprises a complement 3 inhibitor (C3i) or a complement 3 (C3) degraded fragment. In some embodiments, the C3 degraded fragment comprises a C3a, C3b, iC3b, C3f, C3c, C3d, C3g, or a combination thereof. In some embodiments, the engineered polynucleotide comprises a vector such as an AAV vector. In some embodiments, the engineered polynucleotide encodes a first angiogenesis inhibitor and a second angiogenesis inhibitor. In some embodiments, the engineered polynucleotide encodes an angiogenesis inhibitor comprising a complement 3 inhibitor (C3i). For example, FIG. 1 illustrates a vector construct (e.g., Vector GGG, Vector KTP, Vector KTQ, Vector KTR, Vector KAT, Vector KAA, Vector KAC, Vector KAE, Vector KAG, Vector KAK, Vector KAM, or Vector KAP) encoding the complement 3 inhibitor. In some embodiments, the engineered polynucleotide encodes a complement 3 inhibitor and a natriuretic peptide (e.g., a C-type natriuretic peptide or an CNP), for example Vector GGE. In some embodiments, the complement 3 inhibitor and the natriuretic peptide are covalently connected. For example, FIGS. 2A and 2B (Vector GAM and Vector GGE) and FIG. 3 (top panel: Vector GKR sCD59-EVQL C3i-Fc4-CNP36; and bottom panel: Vector KAR sCD59-DK C3i-Fc4-CNP36) illustrate vector constructs encoding the complement 3 inhibitor and the CNP. In some embodiments, the complement 3 inhibitor comprises at least one modification compared to a comparable wild-type complement 3 inhibitor. In some embodiments, the complement 3 inhibitor comprises an amino acid sequence that is at least 80% identical to any one of SEQ ID NOs: 1-15.

In some embodiments, the engineered polynucleotide encodes one or more angiogenesis inhibitors, where one of the angiogenesis inhibitor comprises the CNP. In some embodiments, the CNP is covalently connected to an antibody or fragment thereof (e.g., a fragment crystallizable region). In some embodiments, the natriuretic peptide comprises an amino acid sequence that is at least 80% identical to any one of SEQ ID NOs: 61-72. In some embodiments, the engineered polynucleotide encodes one or more angiogenesis inhibitors, where one of the angiogenesis inhibitor comprises an inhibitor of a membrane attack complex (MAC). In some embodiments, the inhibitor of the MAC comprises an CD59. For example, FIG. 3 illustrates a vector construct encoding the CD59. In some embodiments, the CD59 comprises an amino acid sequence that is at least 80% identical to any one of SEQ ID NOs: 41-45. In some embodiments, the engineered polynucleotide encodes one or more angiogenesis inhibitors, where one of the angiogenesis inhibitor comprises a collagen or fragment thereof (e.g., an endostatin or fragment thereof). In some embodiments, the endostatin comprises an amino acid sequence that is at least 80% identical to SEQ ID NO: 51. In some embodiments, the engineered polynucleotide encodes one or more angiogenesis inhibitors, where one of the angiogenesis inhibitor comprises an VEGF inhibitor. In some embodiments, the VEGF inhibitor comprises an amino acid sequence that is at least 80% identical to any one of SEQ ID NOs: 81-92.

In some embodiments, the engineered polynucleotide comprises a vector. In some embodiments, the vector is a viral vector. In some embodiments, the viral vector comprises an adeno-associated virus (AAV) vector. In some embodiments, the viral vector encodes a modified viral capsid (e.g., as shown in Table 13 and Table 14). In some embodiments, the AAV vector encodes an engineered AAV capsid. In some embodiments, the engineered AAV capsid comprises an amino acid sequence of any one of SEQ ID NOs: 161-182 and SEQ ID NOs: 191-210. In some embodiments, the engineered AAV capsid comprises the amino acid sequence of SEQ ID NO: 169. In some embodiments, the engineered AAV capsid comprises an amino acid sequence of any one of SEQ ID NOs: 277-303 and SEQ ID NOs: 312-319 and 325-329.

In some embodiments, the engineered polynucleotide encodes a first angiogenesis inhibitor comprising a complement 3 inhibitor and a second angiogenesis inhibitor comprising a CNP. In some embodiments, the first angiogenesis inhibitor comprises the complement 3 inhibitor, and the second angiogenesis inhibitor comprises a CNP36. In some embodiments, the first angiogenesis inhibitor comprises the complement 3 inhibitor, and the second angiogenesis inhibitor comprises an Fc-CNP36. In some embodiments, the engineered polynucleotide encodes a third angiogenesis inhibitor. In some embodiments, the third angiogenesis inhibitor comprises an inhibitor of a membrane attack complex (MAC).

In some embodiments, the engineered polynucleotide encodes a first angiogenesis inhibitor comprising an inhibitor of a CD59 and a second angiogenesis inhibitor comprising a CNP. In some embodiments, the first angiogenesis inhibitor comprises the CD59, and the second angiogenesis inhibitor comprises a CNP36. In some embodiments, the first angiogenesis inhibitor comprises the CD59, and the second angiogenesis inhibitor comprises an Fc-CNP36. In some embodiments, the first angiogenesis inhibitor comprises the CD59, and the second angiogenesis inhibitor comprises a complement 3 inhibitor fused to an Fc-CNP36.

In some embodiments, the engineered polynucleotide encodes a first angiogenesis inhibitor comprising a complement 3 inhibitor and a second angiogenesis inhibitor comprising an endostatin. In some embodiments, the engineered polynucleotide encodes an Fc region flanked by the first angiogenesis inhibitor and the second angiogenesis inhibitor. In some embodiments, the first angiogenesis inhibitor comprises a VEGF inhibitor, and the second angiogenesis inhibitor comprises a complement 3 inhibitor. In some embodiments, the first angiogenesis inhibitor comprises a complement 3 inhibitor, and the second angiogenesis inhibitor comprises an Fc-CNP36, wherein the engineered polynucleotide further encodes a third angiogenesis inhibitor comprising a CD59. In some embodiments, the first angiogenesis inhibitor comprises a complement 3 inhibitor, and the second angiogenesis inhibitor comprises an Fc-CNP36, wherein the engineered polynucleotide further encodes a third angiogenesis inhibitor comprising a CD59. In some embodiments, the first angiogenesis inhibitor comprises a CD59, and the second angiogenesis inhibitor comprises a complement 3 inhibitor, wherein the engineered polynucleotide further encodes a third angiogenesis inhibitor comprising a Fc-CNP36. In some embodiments, the first angiogenesis inhibitor comprises a CD59, and the second angiogenesis inhibitor comprises an endostatin, wherein the engineered polynucleotide further encodes a third angiogenesis inhibitor comprising a complement 3 inhibitor. In some embodiments, the first angiogenesis inhibitor comprises a CD59, and the second angiogenesis inhibitor comprises a VEGF inhibitor, wherein the engineered polynucleotide further encodes a third angiogenesis inhibitor comprising a complement 3 inhibitor. In some embodiments, the first angiogenesis inhibitor comprises a complement 3 inhibitor, and the second angiogenesis inhibitor comprises an endostatin, wherein the engineered polynucleotide further encodes a third angiogenesis inhibitor comprising a CD59. In some embodiments, the first angiogenesis inhibitor, the second angiogenesis inhibitor, and the third angiogenesis inhibitor is not a VEGF inhibitor. In some embodiments, the engineered polynucleotide, upon administered to a subject, inhibits neovascularization in the subject. In some embodiments, the first angiogenesis inhibitor or the second angiogenesis inhibitor, upon administered to the subject, exhibits decreased inhibition of neovascularization in the subject compared to inhibition of neovascularization caused by a VEGF inhibitor.

Described herein, in some aspects, is an engineered polypeptide comprising a first angiogenesis inhibitor and a second angiogenesis inhibitor. In some embodiments, the first angiogenesis inhibitor and the second angiogenesis inhibitor are covalently connected by a linker. In some embodiments, the first angiogenesis inhibitor comprises a complement 3 inhibitor. In some embodiments, the complement 3 inhibitor comprises an amino acid sequence that is at least 80% identical to any one of SEQ ID NOs: 1-15. In some embodiments, the first angiogenesis inhibitor comprises an inhibitor of a membrane attack complex (MAC). In some embodiments, the inhibitor of the MAC comprises CD59 comprising an amino acid sequence that is at least 80% identical to any one of SEQ ID NOs: 41-45. In some embodiments, the inhibitor of the MAC comprises CD59 comprising an amino acid sequence that is at least 80% identical to any one of SEQ ID NOs: 312-319. In some embodiments, the inhibitor of the MAC comprises CD59 comprising an amino acid sequence that is at least 80% identical to any one of SEQ ID NOs: 325-329. In some embodiments, the second angiogenesis inhibitor comprises a natriuretic peptide. In some embodiments, the natriuretic peptide is covalently connected to an antibody or fragment thereof. In some embodiments, the antibody or fragment thereof comprises a fragment crystallizable (Fc) region. In some embodiments, the natriuretic peptide comprises an amino acid sequence that is at least 80% identical to any one of SEQ ID NOs: 61-72. In some embodiments, the second angiogenesis inhibitor comprises a collagen or fragment thereof (e.g., an endostatin or fragment thereof). In some embodiments, the second angiogenesis inhibitor comprises an amino acid sequence that is at least 80% identical to SEQ ID NO: 51. In some embodiments, the second angiogenesis inhibitor comprises a VEGF inhibitor. In some embodiments, the VEGF inhibitor comprises an amino acid sequence that is at least 80% identical to any one of SEQ ID NOs: 81-92.

Described herein, in some aspects, is a method for treating a disease or condition in a subject by administering an engineered polynucleotide or an engineered polypeptide to the subject.

In some embodiments, the method comprises once of the administering being curative of the disease or condition. In some embodiments, the method does not comprise daily administration. In some embodiments, the disease or condition comprises an ocular disease. In some embodiments, the ocular disease comprises ocular ischemic syndrome, proliferative retinopathies, neovascular glaucoma (NG), glaucoma, traumatic glaucoma, uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

### Engineered Polynucleotide

Described herein, in some aspects, is an engineered polynucleotide comprising one or more expression cassettes, the one or more expression cassettes encoding a first angiogenesis inhibitor and a second angiogenesis inhibitor. In some embodiments, the engineered polynucleotide comprises one or more expression cassettes for expressing the one or more angiogenesis inhibitors. In some embodiments, the one or more expression cassettes encode a contiguous polypeptide. In some embodiments, the contiguous polypeptide comprises a protease peptide sequence. In some embodiments, the protease peptide sequence is cleavable by a protease expressed endogenously in a cell. Non-limiting example of the protease can include a serine endoprotease, an aspartic endoprotease, a cysteine thiol endoprotease, a metalloendoprotease, or a glutamic acid and threonine endoprotease. In some embodiments, the protease peptide sequence is cleavable by a serine endoprotease. In some embodiments, the protease peptide sequence is cleavable by Furin. In some embodiments, the contiguous polypeptide comprises a protease cleavable sequence. In some embodiments, the protease cleavable sequence can be cleaved by any one of the proteases described herein. In some embodiments, the protease cleavable sequence can be cleaved by Furin. In some embodiments, the contiguous polypeptide comprises a self-cleaving polypeptide sequence. In some embodiments, the self-cleaving polypeptide sequence comprises a 2A self-cleaving peptide sequence. Non-limiting examples of the 2A self-cleaving peptide sequence can include T2A, P2A, E2A, F2A, or a combination thereof. In some embodiments, the self-cleaving polypeptide sequence comprises a F2A peptide sequence. In some embodiments, the contiguous polypeptide comprises a protease cleavable sequence and a self-cleaving polypeptide sequence. For example, the contiguous polypeptide described herein can comprise a Furin-F2A fusion polypeptide sequence. In some embodiments, the engineered polynucleotide comprises a viral vector such as an AAV vector.

In some embodiments, the engineered polynucleotide comprises one or more promoters or internal ribosome entry sites (IRES). In some embodiments, the expression cassette comprises one or more promoters or IRES. In some embodiments, the expression cassette is under expression control of a promoter. In some embodiments, the expression cassette is under expression control of a promoter. In some embodiments, expression cassette can further exert expression control via at least one IRES.

In some embodiments, the engineered polynucleotide comprises at least two, at least three, at least four, at least five, or more expression cassettes. In some embodiments, the engineered polynucleotide comprises two expression cassettes. In some embodiments, the one or more angiogenesis inhibitors are each encoded from an expression cassette of the one or more expression cassettes.

In some embodiments, the engineered polynucleotide encodes a first angiogenesis inhibitor and a second angiogenesis inhibitor. In some embodiments, the first angiogenesis inhibitor comprises a complement 3 inhibitor (C3i). In some embodiments, the engineered polynucleotide encodes a complement 3 inhibitor covalently connected to a natriuretic peptide. In some embodiments, the complement 3 inhibitor (C3i) comprises an amino acid sequence that is at least 75% identical, at least 80% identical, at least 85% identical, at least 90% identical, at least 95% identical, or at least 99% identical to any one of SEQ ID NOs: 1-15 (Table 10). In some embodiments, the complement 3 inhibitor (C3i) comprises an amino acid sequence that is any one of SEQ ID NOs: 1-15. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 1. In some embodiments, the complement 3 inhibitor (C3i) comprises an amino acid sequence that is SEQ ID NO: 1. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 2. In some embodiments, the complement 3 inhibitor (C3i) comprises an amino acid sequence that is SEQ ID NO: 2. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 3. In some embodiments, the complement 3 inhibitor (C3i) comprises an amino acid sequence that is SEQ ID NO: 3. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 4. In some embodiments, the complement 3 inhibitor (C3i) comprises an amino acid sequence that is SEQ ID NO: 4. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 5. In some embodiments, the complement 3 inhibitor (C3i) comprises an amino acid sequence that is SEQ ID NO: 5. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 6. In some embodiments, the complement 3 inhibitor (C3i) comprises an amino acid sequence that is SEQ ID NO: 6. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 7. In some embodiments, the complement 3 inhibitor (C3i) comprises an amino acid sequence that is SEQ ID NO: 7. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 8. In some embodiments, the complement 3 inhibitor (C3i) comprises an amino acid sequence that is SEQ ID NO: 8. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 9. In some embodiments, the complement 3 inhibitor (C3i) comprises an amino acid sequence that is SEQ ID NO: 9. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 10. In some embodiments, the complement 3 inhibitor (C3i) comprises an amino acid sequence that is SEQ ID NO: 10. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 11. In some embodiments, the complement 3 inhibitor (C3i) comprises an amino acid sequence that is SEQ ID NO: 11. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 12. In some embodiments, the complement 3 inhibitor (C3i) comprises an amino acid sequence that is SEQ ID NO: 12. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 13. In some embodiments, the complement 3 inhibitor (C3i) comprises an amino acid sequence that is SEQ ID NO: 13. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 14. In some embodiments, the complement 3 inhibitor (C3i) comprises an amino acid sequence that is SEQ ID NO: 14. In some embodiments, the complement 3 inhibitor (C3i) comprises an amino acid sequence that is at least 75% identical, at least 80% identical, at least 85% identical, at least 90% identical, at least 95% identical, or at least 99% identical to SEQ ID NO: 15. In some embodiments, the complement 3 inhibitor (C3i) comprises an amino acid sequence that is SEQ ID NO: 15.

In some embodiments, the complement 3 inhibitor (C3i) comprises an amino acid sequence that is at 8 contiguous amino acids, at least 10 contiguous amino acids, or at least 12 contiguous amino acids of any one of SEQ ID NOs: 1-15. In some embodiments, the complement 3 inhibitor (C3i) comprises an amino acid sequence that is at 8 contiguous amino acids, at least 10 contiguous amino acids, or at least 12 contiguous amino acids of SEQ ID NO: 1. In some embodiments, the complement 3 inhibitor (C3i) comprises an amino acid sequence that is at 8 contiguous amino acids, at least 10 contiguous amino acids, or at least 12 contiguous amino acids of SEQ ID NO: 2. In some embodiments, the complement 3 inhibitor (C3i) comprises an amino acid sequence that is at 8 contiguous amino acids, at least 10 contiguous amino acids, or at least 12 contiguous amino acids of SEQ ID NO: 3. In some embodiments, the complement 3 inhibitor (C3i) comprises an amino acid sequence that is at 8 contiguous amino acids, at least 10 contiguous amino acids, or at least 12 contiguous amino acids of SEQ ID NO: 4. In some embodiments, the complement 3 inhibitor (C3i) comprises an amino acid sequence that is at 8 contiguous amino acids, at least 10 contiguous amino acids, or at least 12 contiguous amino acids of SEQ ID NO: 5. In some embodiments, the complement 3 inhibitor (C3i) comprises an amino acid sequence that is at 8 contiguous amino acids, at least 10 contiguous amino acids, or at least 12 contiguous amino acids of SEQ ID NO: 6. In some embodiments, the complement 3 inhibitor (C3i) comprises an amino acid sequence that is at 8 contiguous amino acids, at least 10 contiguous amino acids, or at least 12 contiguous amino acids of SEQ ID NO: 7. In some embodiments, the complement 3 inhibitor (C3i) comprises an amino acid sequence that is at 8 contiguous amino acids, at least 10 contiguous amino acids, or at least 12 contiguous amino acids of SEQ ID NO: 8. In some embodiments, the complement 3 inhibitor (C3i) comprises an amino acid sequence that is at 8 contiguous amino acids, at least 10 contiguous amino acids, or at least 12 contiguous amino acids of SEQ ID NO: 9. In some embodiments, the complement 3 inhibitor (C3i) comprises an amino acid sequence that is at 8 contiguous amino acids, at least 10 contiguous amino acids, or at least 12 contiguous amino acids of SEQ ID NO: 10. In some embodiments, the complement 3 inhibitor (C3i) comprises an amino acid sequence that is at 8 contiguous amino acids, at least 10 contiguous amino acids, or at least 12 contiguous amino acids of SEQ ID NO: 11. In some embodiments, the complement 3 inhibitor (C3i) comprises an amino acid sequence that is at 8 contiguous amino acids, at least 10 contiguous amino acids, or at least 12 contiguous amino acids of SEQ ID NO: 12. In some embodiments, the complement 3 inhibitor (C3i) comprises an amino acid sequence that is at 8 contiguous amino acids, at least 10 contiguous amino acids, or at least 12 contiguous amino acids of SEQ ID NO: 13. In some embodiments, the complement 3 inhibitor (C3i) comprises an amino acid sequence that is at 8 contiguous amino acids, at least 10 contiguous amino acids, or at least 12 contiguous amino acids of SEQ ID NO: 14. In some embodiments, the complement 3 inhibitor (C3i) comprises an amino acid sequence that is at 8 contiguous amino acids, at least 10 contiguous amino acids, or at least 12 contiguous amino acids of SEQ ID NO: 15.

TABLE 10
Exemplary complement 3 inhibitor (C3i) amino acid sequences

| SEQ ID | | | Vector |
|---|---|---|---|
| NO | Amino acid sequence | Notes | name |
| 1 | GICVVQDWGHHRCT | C3i 13-mer | |
| 2 | GICVWQDWGAHRCTN | C3i 15-mer | Vector |
| | | | GGG |
| 3 | GICVWQDWGAHRCAN | T14A (N15) | Vector |
| | | | KTP |
| 4 | GYICVWQDWGAHRCTN | + 2Y (N15) | Vector |
| | | | KTQ |
| 5 | GYICVWQDWGAHRCAN | +2Y; T14A | Vector |
| | | (N15) | KTR |
| 6 | GICVWQDWGAHRCT | - N15 | Vector |
| | | | KAT |
| 7 | GICVWQDWGAHRCA | T14A; - N15 | Vector |
| | | | KAA |
| 8 | GYICVWQDWGAHRCT | + 2Y; - N15 | Vector |
| | | | KAC |
| 9 | GYICVWQDWGAHRCA | +2Y; T14A; - | Vector |
| | | N15 | KAE |
| 10 | GICVWQDWGAHRCTQ | N15Q | Vector |
| | | | KAG |
| 11 | GICVWQDWGAHRCAQ | T14A; N15Q | Vector |
| | | | KAK |
| 12 | GYICVWQDWGAHRCTQ | + 2Y; N15Q | Vector |
| | | | KAM |
| 13 | GYICVWQDWGAHRCAQ | +2Y; T14A; | Vector |
| | | N15Q | KAP |
| 14 | DKGICVWQDWGAHRCTNGGGGSGGGGS | | |
| 15 | EVQLGICVWQDWGAHRCTNGGGGSGGGGS | | |

In some embodiments, the engineered polynucleotide encoding the complement 3 inhibitor comprises a nucleic acid sequence that is at least 75% identical, at least 80% identical, at least 85% identical, at least 90% identical, at least 95% identical, or at least 99% identical to any one of SEQ ID NOs: 20-33 (Table 11). In some embodiments, the complement 3 inhibitor (C3i) is encoded by a nucleic acid sequence that is any one of SEQ ID NOs: 20-33. In some embodiments, the complement 3 inhibitor (C3i) is encoded by a nucleic acid sequence that is at least 50 contiguous nucleotide bases, at least 60 contiguous nucleotide bases, at least 70 contiguous nucleotide bases, or at least 50 contiguous nucleotide bases of any one of SEQ ID NOs: 20-33.

TABLE 11
Exemplary nucleic acid sequence encoding complement 3 inhibitor

| SEQ ID | | | Vector |
|---|---|---|---|
| NO | Nucleic acid sequence | Notes | name |
| 20 | GACAAGGGCATCTGCGTGTGGCAGGACTGGGG | | |
| | CGCCCACCGC | | |
| | TGCACCAACGGCGGCGGCGGCAGCGGCGGCG | | |
| | GCGGCAGC | | |
| 21 | GACAAGGGCATCTGCGTGTGGCAGGACTGGGG | | |
| | CGCCCACCGCTGCACCAA | | |
| | CGGCGGAGGCGGAAGCGGCGGAGGCGGATCT | | |
| 22 | ggcatctgcgtgtggcaggactggggcgcccaccgctgcaccaac | C3i | Vector |
| | | 15-mer | GGG |
| 23 | ggcatctgcgtgtggcaggactggggcgcccaccgctgcGccaac | T14A | Vector |
| | | (N15) | KTP |
| 24 | ggcTACatctgcgtgtggcaggactggggcgcccaccgctgcaccaac | +2Y | Vector |
| | | (N15) | KTQ |
| 25 | ggcTACatctgcgtgtggcaggactggggcgcccaccgctgcGccaac | +2Y; | Vector |
| | | T14A | KTR |
| | | (N15) | |
| 26 | ggcatctgcgtgtggcaggactggggcgcccaccgctgcacc | −N15 | Vector |
| | | | KAT |
| 27 | ggcatctgcgtgtggcaggactggggcgcccaccgctgcGcc | T14A; | Vector |
| | | −N15 | KAA |
| 28 | ggcTACatctgcgtgtggcaggactggggcgcccaccgctgcacc | +2Y; | Vector |
| | | −N15 | KAC |
| 29 | ggcTACatctgcgtgtggcaggactggggcgcccaccgctgcGcc | +2Y; | Vector |
| | | T14A; | KAE |
| | | −N15 | |
| 30 | ggcatctgcgtgtggcaggactggggcgcccaccgctgcaccCAG | N15Q | Vector |
| | | | KAG |
| 31 | ggcatctgcgtgtggcaggactggggcgcccaccgctgcGccCAG | T14A; | Vector |
| | | N15Q | KAK |
| 32 | ggcTACatctgcgtgtggcaggactggggcgcccaccgctgcaccCAG | +2Y; | Vector |
| | | N15Q | KAM |
| 33 | ggcTACatctgcgtgtggcaggactggggcgcccaccgctgcGccCAG | +2Y; | Vector |
| | | T14A; | KAP |
| | | N15Q | |

In some embodiments, the engineered polynucleotide encodes a natriuretic peptide or a natriuretic peptide fusion protein (e.g., a CNP-Fc fusion protein described herein). In some embodiments, the natriuretic peptide is a CNP. In some embodiments, the CNP is covalently connected to a complement 3 inhibitor. In some embodiments, the CNP is covalently connected to a complement 3 inhibitor by a linker. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to any one of SEQ ID NOs: 61-72 (Table 12). In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 61. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 62. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 63. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 64. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 65. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 66. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 67. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 68. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 69. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 70. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 71. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to SEQ ID NO: 72. In some embodiments, the natriuretic peptide or the natriuretic peptide fusion protein comprises an amino acid sequence that is any one of SEQ ID NOs: 61-72.

TABLE 12
Exemplary natriuretic peptide or the natriuretic peptide fusion
protein amino acid sequences

| SEQ ID | | |
|---|---|---|
| NO | Amino acid sequence | Notes |
| 61 | MHLSQLLACALLLTLLSLRPSEAKPGAPPKVPRTPPAEELAEPQ | Full |
| | AAGGGQKKGDKAPGGGGANLKGDRSRLLRDLRVDTKSRAAW | length |
| | ARLLQEHPNARKYKGANKKGLSKGCFGLKLDRIGSMSGLGC | CNP |
| 62 | GDRSRLLRDLRVDTKSRAAWARLLQEHPNA | CNP |
| | RKYKGANKKGLSKGCFGLKLDRIGSMSGLGC | precursor |
| | | (CNP-61) |
| 63 | DLRVDTKSRAAWARLLQEHPNARKYKGANKK | CNP-53 |
| | GLSKGCFGLKLDRIGSMSGLGC | |
| 64 | EHPNARKYKGANKKGLSKGCFGLKLDRIGSMSGLGC | CNP-36 |
| 65 | GLSKGCFGLKLDRIGSMSGLGC | CNP-22 |
| 66 | MEFGLSWLFLVAILKGVQC | Antibody |
| | | signal |
| | | peptide |
| 67 | DKTHTCPPCPAPELLGGPSVFLFPPKPKDTLMISRTPEVTCVVVD | IgG1 |
| | VSHEDPEVKFNWYVDGVEVHNAKTKPREEQYNSTYRVVSVLT | |
| | VLHQDWLNGKEYKCKVSNKALPAPIEKTISKAKGQPREPQVYT | |
| | LPPSRDELTKNQVSLTCLVKGFYPSDIAVEWESNGQPENNYKTT | |
| | PPVLDSDGSFFLYSKLTVDKSRWQQGNVFSCSVMHEALHNHY | |
| | TQKSLSLSPGK | |
| 68 | DKRVESKYGPPCPPCPAPEFLGGPSVFLFPPKPKDTLMISRTPEV | IgG4 |
| | TCVVVDVSQEDPEVQFNWYVDGVEVHNAKTKPREEQFNSTYR | |
| | VVSVLTVLHQDWLNGKEYKCKVSNKGLPSSIEKTISKAKGQPR | |
| | EPQVYTLPPSQEEMTKNQVSLTCLVKGFYPSDIAVEWESNGQPE | |
| | NNYKTTPPVLDSDGSFFLYSRLTVDKSRWQEGNVFSCSVMHEA | |
| | LHNHYTQKSLSLSLGK | |
| 69 | MEFGLWLFLVAILKGVQCEHPNARKYKGANKKG | SP- |
| | LSKGCFGLKLDRIGSMSGLGC | CNP36 |
| 70 | MEFGLWLFLVAILKGVQCGLSKGCFGLKLDRIGSMSGLGC | SP- |
| | | CNP22 |
| 71 | MEFGLSWLFLVAILKGVQCDKTHTCPPCPAPELLGGPSVFLFPP | SPVh- |
| | KPKDTLMISRTPEVTCVVVDVSHEDPEVKFNWYVDGVEVHNA | CNP36 |
| | KTKPREEQYNSTYRVVSVLTVLHQDWLNGKEYKCKVSNKALP | |
| | APIEKTISKAKGQPREPQVYTLPPSRDELTKNQVSLTCLVKGFYP | |
| | SDIAVEWESNGQPENNYKTTPPVLDSDGSFFLYSKLTVDKSRW | |
| | QQGNVFSCSVMHEALHNHYTQKSLSLSPGKGGGGSGGGGSGG | |
| | GGSGGGGSEHPNARKYKGANKKGLSKGCFGLKLDRIGSMSGL | |
| | GC | |
| 72 | MEFGLSWLFLVAILKGVQCDKTHTCPPCPAPELLGGPSVFLFPP | SPVh- |
| | KPKDTLMISRTPEVTCVVVDVSHEDPEVKFNWYVDGVEVHNA | CNP22 |
| | KTKPREEQYNSTYRVVSVLTVLHQDWLNGKEYKCKVSNKALP | |
| | APIEKTISKAKGQPREPQVYTLPPSRDELTKNQVSLTCLVKGFYP | |
| | SDIA VEWESNGQPENNYKTTPPVLDSDGSFFLYSKLTVDKSRW | |
| | QQGNVFSCSVMHEALHNHYTQKSLSLSPGKGGGGSGGGGSGG | |
| | GGSGGGGSLSKGCFGLKLDRIGSMSGLGC | |

In some embodiments, the engineered polynucleotide encodes an inhibitor of a membrane attack complex (MAC). In some embodiments, the inhibitor of the MAC comprises CD59. In some embodiments, the CD59 comprises an amino acid sequence that is at least 75% identical, at least 80% identical, at least 85% identical, at least 90% identical, at least 95% identical, or at least 99% identical to any one of SEQ ID NOs: 41-45. (Table 13). In some embodiments, the CD59 comprises an amino acid sequence that is at least 75% identical, at least 80% identical, at least 85% identical, at least 90% identical, at least 95% identical, or at least 99% identical to SEQ ID NO: 41. In some embodiments, the CD59 comprises an amino acid sequence that is at least 75% identical, at least 80% identical, at least 85% identical, at least 90% identical, at least 95% identical, or at least 99% identical to SEQ ID NO: 42. In some embodiments, the CD59 comprises an amino acid sequence that is at least 75% identical, at least 80% identical, at least 85% identical, at least 90% identical, at least 95% identical, or at least 99% identical to SEQ ID NO: 43. In some embodiments, the CD59 comprises an amino acid sequence that is at least 75% identical, at least 80% identical, at least 85% identical, at least 90% identical, at least 95% identical, or at least 99% identical to SEQ ID NO: 44. In some embodiments, the CD59 comprises an amino acid sequence that is at least 75% identical, at least 80% identical, at least 85% identical, at least 90% identical, at least 95% identical, or at least 99% identical to SEQ ID NO: 45. In some embodiments, the CD59 comprises an amino acid sequence that is any one of SEQ ID NOs: 41-45.

TABLE 13
Exemplary CD59 amino acid sequences

| SEQ ID | | |
|---|---|---|
| NO | Amino acid sequence | Notes |
| 41 | MGIQGGSVLFGLLLVLAVFCHSGHSLQCYNCPNPTADCKTAVNCSS | PRO-cd59 |
| | DFDACLITKAGLQVYNKCWKFEHCNFNDVTTRLRENELTYYCCKK | |
| | DLCNFNEQLENGGTSLSEKTVLLLVTPFLAAAWSLHP | |
| 42 | MGIQGGSVLFGLLLVLAVFCHSGHSLQCYNCPNPTADCKTAVNCSS | Soluble |
| | DFDACLITKAGLQVYNKCWKFEHCNFNDVTTRLRENELTYYCCKK | CD59 |
| | DLCNFNEQLEN | |
| 43 | LQCYNCPNPTADCKTAVNCSSDFDACLITKAGLQVYNKCWKFEHC | Membrane |
| | NFNDVTTRLRENELTYYCCKKDLCNFNEQLENGGTSLSEKTVLLLV | anchored |
| | TPFLAAAWSLHP | CD59 |
| | | (mCD59) |
| 44 | LQCYNCPNPTADCKTAVNCSSDFDACLITKAGLQVYNKCWKFEHC | Native |
| | NFNDVTTRLRENELTYYCCKKDLCNFNEQLEN | soluble |
| | | CD59 |
| | | (nsCD59) |
| 45 | EVQLQCYNCPNPTADCKTAVNCSSDFDACLITKAGLQVYNKCWKF | Vh-soluble |
| | EHCNFNDVTTRLRENELTYYCCKKDLCNFNEQLEN | CD59 |
| | | (vsCD59) |

In some embodiments, the engineered polynucleotide encodes an inhibitor of a membrane attack complex (MAC). In some embodiments, the inhibitor of the MAC comprises CD59. In some embodiments, the CD59 comprises an amino acid sequence that is at least 75% identical, at least 80% identical, at least 85% identical, at least 90% identical, at least 95% identical, or at least 99% identical to any one of SEQ ID NOs: 312-319. (Table 40). In some embodiments, the CD59 comprises an amino acid sequence that is at least 75% identical, at least 80% identical, at least 85% identical, at least 90% identical, at least 95% identical, or at least 99% identical to SEQ ID NO: 312. In some embodiments, the CD59 comprises an amino acid sequence that is at least 75% identical, at least 80% identical, at least 85% identical, at least 90% identical, at least 95% identical, or at least 99% identical to SEQ ID NO: 313. In some embodiments, the CD59 comprises an amino acid sequence that is at least 75% identical, at least 80% identical, at least 85% identical, at least 90% identical, at least 95% identical, or at least 99% identical to SEQ ID NO: 314. In some embodiments, the CD59 comprises an amino acid sequence that is at least 75% identical, at least 80% identical, at least 85% identical, at least 90% identical, at least 95% identical, or at least 99% identical to SEQ ID NO: 315. In some embodiments, the CD59 comprises an amino acid sequence that is at least 75% identical, at least 80% identical, at least 85% identical, at least 90% identical, at least 95% identical, or at least 99% identical to SEQ ID NO: 316. In some embodiments, the CD59 comprises an amino acid sequence that is at least 75% identical, at least 80% identical, at least 85% identical, at least 90% identical, at least 95% identical, or at least 99% identical to SEQ ID NO: 317. In some embodiments, the CD59 comprises an amino acid sequence that is at least 75% identical, at least 80% identical, at least 85% identical, at least 90% identical, at least 95% identical, or at least 99% identical to SEQ ID NO: 318. In some embodiments, the CD59 comprises an amino acid sequence that is at least 75% identical, at least 80% identical, at least 85% identical, at least 90% identical, at least 95% identical, or at least 99% identical to SEQ ID NO: 319. In some embodiments, the CD59 comprises an amino acid sequence that is any one of SEQ ID NOs: 312-319.

In some embodiments, the engineered polynucleotide encodes an inhibitor of a membrane attack complex (MAC). In some embodiments, the inhibitor of the MAC comprises CD59. In some embodiments, the CD59 comprises an amino acid sequence that is at least 75% identical, at least 80% identical, at least 85% identical, at least 90% identical, at least 95% identical, or at least 99% identical to any one of SEQ ID NOs: 325-329. (Table 50). In some embodiments, the CD59 comprises an amino acid sequence that is at least 75% identical, at least 80% identical, at least 85% identical, at least 90% identical, at least 95% identical, or at least 99% identical to SEQ ID NO: 325. In some embodiments, the CD59 comprises an amino acid sequence that is at least 75% identical, at least 80% identical, at least 85% identical, at least 90% identical, at least 95% identical, or at least 99% identical to SEQ ID NO: 326. In some embodiments, the CD59 comprises an amino acid sequence that is at least 75% identical, at least 80% identical, at least 85% identical, at least 90% identical, at least 95% identical, or at least 99% identical to SEQ ID NO: 327. In some embodiments, the CD59 comprises an amino acid sequence that is at least 75% identical, at least 80% identical, at least 85% identical, at least 90% identical, at least 95% identical, or at least 99% identical to SEQ ID NO: 328. In some embodiments, the CD59 comprises an amino acid sequence that is at least 75% identical, at least 80% identical, at least 85% identical, at least 90% identical, at least 95% identical, or at least 99% identical to SEQ ID NO: 329. In some embodiments, the CD59 comprises an amino acid sequence that is any one of SEQ ID NOs: 325-329.

TABLE 45
Exemplary CD59 amino acid sequences

| SEQ ID | | |
|---|---|---|
| NO | Amino acid sequence | Notes |
| 312 | MEFGLSWLFLVAILKGVQCDKLQCYNCPNPTADCKTAVNCSSD | Vh(DK) sCD59 |
| | FDACLITKAGLQVYNKCWKFEHCNFNDVTTRLRENELTYYCC | |
| | KKDLCNFNEQLEN | |
| 313 | MGIQGGSVLFGLLLVLAVFCHSGHSLQCYNCPNPTADCKTAVN | vsCD59-6 × His |
| | CSSDFDACLITKAGLQVYNKCWKFEHCNFNDVTTRLRENELTY | |
| | YCCKKDLCNFNEQLENHHHHHH | |
| 314 | MGIQGGSVLFGLLLVLAVFCHSGHSLQCYNCPNPTADCKTAVQ | vsCD59 N18Q- |
| | CSSDFDACLITKAGLQVYNKCWKFEHCNFNDVTTRLRENELTY | 6 × His |
| | YCCKKDLCNFNEQLENHHHHHH | |
| 315 | MGIQGGSVLFGLLLVLAVFCHSGHSLQCYNCPNPTADCKTAVN | vsCD59 Q343E- |
| | CSSDFDACLITKAGLEVYNKCWKFEHCNFNDVTTRLRENELTY | 6 × His |
| | YCCKKDLCNFNEQLENHHHHHH | |
| 316 | MGIQGGSVLFGLLLVLAVFCHSGHSLQCYNCPNPTADCKTAVN | vsCD59 K38R- |
| | CSSDFDACLITKAGLQVYNRCWKFEHCNFNDVTTRLRENELTY | 6 × His |
| | YCCKKDLCNFNEQLENHHHHHH | |
| 317 | MGIQGGSVLFGLLLVLAVFCHSGHSLQCYNCPNPTADCKTAVN | vsCD59 Q343E, |
| | CSSDFDACLITKAGLEVYNRCWKFEHCNFNDVTTRLRENELTY | K38R-6 × His |
| | YCCKKDLCNFNEQLENHHHHHH | |
| 318 | MGIQGGSVLFGLLLVLAVFCHSGHSLQCYNCPNPTADCKTAVQ | vsCD59 N18Q, |
| | CSSDFDACLITKAGLQVYNRCWKFEHCNFNDVTTRLRENELTY | K38R-6 × His |
| | YCCKKDLCNFNEQLENHHHHHH | |
| 319 | MGIQGGSVLFGLLLVLAVFCHSGHSLQCYNCPNPTADCKTAVQ | vsCD59 N18Q, |
| | CSSDFDACLITKAGLEVYNRCWKFEHCNFNDVTTRLRENELTY | Q33E, K38R-6 × His |
| | YCCKKDLCNFNEQLENHHHHHH | |
| 325 | MGIQGGSVLFGLLLVLAVFCHSGHSLQCYNCPNPTADCKTAVN | sCD59 L33E-6 x His |
| | CSSDFDACLITKAGEQVYNKCWKFEHCNFNDVTTRLRENELTY | |
| | YCCKKDLCNFNEQLENHHHHHH | |
| 326 | MGIQGGSVLFGLLLVLAVFCHSGHSLQCYNCPNPTADCKTAVN | sCD59 S20G- |
| | CGSDFDACLITKAGLQVYNKCWKFEHCNFNDVTTRLRENELTY | 6 × His |
| | YCCKKDLCNFNEQLENHHHHHH | |
| 327 | MGIQGGSVLFGLLLVLAVFCHSGHSLQCYNCPNPTADCKTAVN | sCD59 T51G- |
| | CSSDFDACLITKAGLQVYNKCWKFEHCNFNDVGTRLRENELTY | 6 × His |
| | YCCKKDLCNFNEQLENHHHHHH | |
| 328 | MGIQGGSVLFGLLLVLAVFCHSGHSLQCYNCPNPTADCKTAVN | sCD59 S20G, |
| | CGSDFDACLITKAGLQVYNKCWKFEHCNFNDVGTRLRENELT | T51G-6 × His |
| | YYCCKKDLCNFNEQLENHHHHHH | |
| 329 | MGIQGGSVLFGLLLVLAVFCHSGHSLQCYNCPNPTADCKTAVQ | SCD59 N18Q, |
| | CSSDFDACLITKAGLEVYNKCWKFEHCNFNDVTTRLRENELTY | Q34E-6 × His |
| | YCCKKDLCNFNEQLENHHHHHH | |

In some embodiments, the engineered polynucleotide encodes a collagen or fragment thereof. In some embodiments, the collagen or fragment thereof comprises an endostatin or fragment thereof. In some embodiments, the endostatin or fragment thereof comprises an amino acid sequence that is at least 75% identical, at least 80% identical, at least 85% identical, at least 90% identical, at least 95% identical, or at least 99% identical to SEQ ID NO: 51: MHSHRDFQPVLHLVALNSPLSGGMRGIRGADFQCFQQARAVGLAGTFRAFLSSRLQDL YSIVRRADRAAVPIVNLKDELLFPSWEALFSGSEGPLKPGARIFSFDGKDVLRHPTWPQK SVWHGSDPNGRRLTESYCETWRTEAPSATGQASSLLGGRLLGQSAASCHHAYIVLCIEN SFMTASK. In some embodiments, the endostatin or fragment thereof comprises an amino acid sequence that is SEQ ID NO: 51.

In some embodiments, the engineered polynucleotide encodes an VEGF inhibitor. In some embodiments, the VEGF inhibitor comprises an inhibitory RNA for targeting and degrading the VEGF transcript. In some embodiments, the VEGF inhibitor comprises an antibody or a fragment thereof. In some embodiments, the VEGF antibody binds to VEGF to decrease neovascularization signaling comprising the VEGF signaling transduction pathway. In some embodiments, the VEGF antibody binds to VEGF-A, VEGF-B, VEGF-C, VEGF-D, or a combination thereof. In some embodiments, the VEGF antibody binds to one or more isoforms of VEGF-A, including VEGF121, VEGF145, VEGF148, VEGF162, VEGF165, VEGF165b, VEGF183, VEGF189, or VEGF206. In some embodiments, the antibody comprises monovalent Fab′, a divalent Fab2, a F (ab)′3 fragments, a single-chain variable fragment (scFv), a bis-scFv, (scFv) 2, a diabody, a minibody, a nanobody, a triabody, a tetrabody, a disulfide stabilized Fv protein (“dsFv”), a single-domain antibody (sdAb), an Ig NAR, a camelid antibody, or a combination thereof, a binding fragment thereof, or a chemically modified derivative thereof. Non-limiting examples of VEGF antibodies include ranibizumab or bevacizumab. In some embodiments, the VEGF antibody comprises a polypeptide sequence that is at least 70%, at least 75%, at least 80%, at least 85%, at least 90%, at least 95%, at least 99%, or more identical to any one of SEQ ID NOs: 81-87 (Table 14), or a combination thereof, or a fragment thereof.

TABLE 14
Exemplary amino acid sequences of VEGF antibodies

| SEQ ID | |
|---|---|
| NO | Amino acid sequence of VEGF antibody |
| 81 | EVQLVESGGGLVQPGGSLRLSCAASGYDFTHYGMNWVRQAPGKGLEWV |
| | GWINTYTGEPTYAADFKRRFTFSLDTSKSTAYLQMNSLRAEDTAVYYCA |
| | KYPYYYGTSHWYFDVWGQGTLVTVSS |
| 82 | DIQLTQSPSSLSASVGDRVTITCSASQDISNYLNWYQQKPGKAPKVLIYFTS |
| | SLHSGVPSRFSGSGSGTDFTLTISSLQPEDFATYYCQQYSTVPWTFGQGTK |
| | VEIKRTVAA |
| 83 | EVQLVESGGGLVQPGGSLRLSCAASGYDFTHYGMNWVRQAPGKGLEWV |
| | GWINTYTGEPTYAADFKRRFTFSLDTSKSTAYLQMNSLRAEDTAVYYCA |
| | KYPYYYGTSHWYFDVWGQGTLVTVSSGGGGSGGGGSGGGGSGGGGSDI |
| | QLTQSPSSLSASVGDRVTITCSASQDISNYLNWYQQKPGKAPKVLIYFTSS |
| | LHSGVPSRFSGSGSGTDFTLTISSLQPEDFATYYCQQYSTVPWTFGQGTKV |
| | EIKRTVAA |
| 84 | EVQLVESGGGLVQPGGSLRLSCAASGYDFTHYGMNWVRQAPGKGLEWV |
| | GWINTYTGEPTYAADFKRRFTFSLDTSKSTAYLQMNSLRAEDTAVYYCA |
| | KYPYYYGTSHWYFDVWGQGTLVTVSSASTKGPSVFPLAPSSKSTSGGTA |
| | ALGCLVKDYFPEPVTVSWNSGALTSGVHTFPAVLQSSGLYSLSSVVTVPSS |
| | SLGTQTYICNVNHKPSNTKVDKKVEPKSCDKTHL |
| 85 | DIQLTQSPSSLSASVGDRVTITCSASQDISNYLNWYQQKPGKAPKVLIYFTS |
| | SLHSGVPSRFSGSGSGTDFTLTISSLQPEDFATYYCQQYSTVPWTFGQGTK |
| | VEIKRTVAAPSVFIFPPSDEQLKSGTASVVCLLNNFYPREAKVQWKVDNA |
| | LQSGNSQESVTEQDSKDSTYSLSSTLTLSKADYEKHKVYACEVTHQGLSS |
| | PVTKSFNRGEC |
| 86 | DIQLTQSPSSLSASVGDRVTITCSASQDISNYLNWYQQKPGKAPKVLIYFTS |
| | SLHSGVPSRFSGSGSGTDFTLTISSLQPEDFATYYCQQYSTVPWTFGQGTK |
| | VEIKRTVAAGGGGSGGGGSGGGGSGGGGSEVQLVESGGGLVQPGGSLRL |
| | SCAASGYDFTHYGMNWVRQAPGKGLEWVGWINTYTGEPTYAADFKRRF |
| | TFSLDTSKSTAYLQMNSLRAEDTAVYYCAKYPYYYGTSHWYFDVWGQG |
| | TLVTVSS |
| 87 | MEIVMTQSPSTLSASVGDRVIITCQASEIIHSWLAWYQQKPGKAPKLLIYL |
| | ASTLASGVPSRFSGSGSGAEFTLTISSLQPDDFATYYCQNVYLASTNGANF |
| | GQGTKLTVLGGGGGSGGGGSGGGGSGGGGSEVQLVESGGGLVQPGGSL |
| | RLSCTASGFSLTDYYYMTWVRQAPGKGLEWVGFIDPDDDPYYATWAKG |
| | RFTISRDNSKNTLYLQMNSLRAEDTAVYYCAGGDHNSGWGLDIWGQGTL |
| | VTVSS |

In some embodiments, the VEGF inhibitor is not an antibody. For example, the VEGF inhibitor described herein can comprise a VEGF receptor, a combination of VEGF receptors, or a fragment thereof for binding to VEGF for inhibiting or decreasing VEGF signaling transduction pathway. VEGF receptor can include a VEGF receptor 1 (FLT1), a VEGF receptor 2 (KDR/FLK1), a VEGF receptor 3 (FLT4), a fragment thereof, or a combination thereof. In some embodiments, the VEGF receptor can be a soluble VEGF receptor. For example, the soluble VEGF receptor can comprise a soluble VEGFR1, a soluble VEGFR2, a soluble VEGFR3, a soluble fragment thereof, or a combination thereof. In some embodiments, the non-antibody VEGF inhibitor comprises at least one of FLT1, KDR/FLK1, FLT4, a fragment thereof, or a combination thereof. In some embodiments, the non-antibody VEGF inhibitor comprises at least one of soluble FLT1, soluble KDR/FLK1, soluble FLT4, a fragment thereof, or a combination thereof. In some embodiments, the non-antibody inhibitor VEGF comprises a VEGF-Trap. In some embodiments, the non-antibody VEGF inhibitor comprises a polypeptide sequence that is at least 70%, at least 75%, at least 80%, is at least 85%, at least 90%, at least 95%, at least 99%, or more identical to any one of SEQ ID NOs: 88-92 (Table 15).

TABLE 15
Exemplary amino acid sequence of non-antibody VEGF inhibitor

| SEQ ID | |
|---|---|
| NO | Non-antibody VEGF inhibitor |
| 88 | LPAQVAFTPYAPEPGSTCRLREYYDQTAQMCCSKCSPGQHAKVFCTKTSD |
| | TVCDSCEDSTYTQLWNWVPECLSCGSRCSSDQVETQACTREQNRICTCRP |
| | GWYCALSKQEGCRLCAPLRKCRPGFGVARPGTETSDVVCKPCAPGTFSNT |
| | TSSTDICRPHQICNVVAIPGNASMDAVCTSTSPTRSMAPGAVHLPQPVSTRS |
| | QHTQPTPEPSTAPSTSFLLPMGPSPPAEGSTGDEPKSCDKTHTCPPCPAPELL |
| | GGPSVFLFPPKPKDTLMISRTPEVTCVVVDVSHEDPEVKFNWYVDGVEVH |
| | NAKTKPREEQYNSTYRVVSVLTVLHQDWLNGKEYKCKVSNKALPAPIEKT |
| | ISKAKGQPREPQVYTLPPSREEMTKNQVSLTCLVKGFYPSDIAVEWESNGQ |
| | PENNYKTTPPVLDSDGSFFLYSKLTVDKSRWQQGNVFSCSVMHEALHNHY |
| | TQKSLSLSPGK |
| 89 | DTGRPFVEMYSEIPEIIHMTEGRELVIPCRVTSPNITVTLKKFPLDTLIPDGKR |
| | IIWDSRKGFIISNATYKEIGLLTCEATVNGHLYKTNYLTHRQTNTIIDVVLSP |
| | SHGIELSVGEKLVLNCTARTELNVGIDENWEYPSSKHQHKKLVNRDLKTQ |
| | SGSEMKKFLSTLTIDGVTRSDQGLYTCAASSGLMTKKNSTFVRVHEK |
| 90 | DTGRPFVEMYSEIPEIIHMTEGRELVIPCRVTSPNITVTLKKFPLDTLIPDGKR |
| | IIWDSRKGFIISNATYKEIGLLTCEATVNGHLYKTNYLTHRQTNTIIDVVLSP |
| | SHGIELSVGEKLVLNCTARTELNVGIDENWEYPSSKHQHKKLVNRDLKTQ |
| | SGSEMKKFLSTLTIDGVTRSDQGLYTCAASSGLMTKKNSTFVRVHEKDKT |
| | HTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNVFSC |
| | SVMHEALHNHYTQKSLSLSPGK |
| 91 | SDTGRPFVEMYSEIPEIIHMTEGRELVIPCRVTSPNITVTLKKFPLDTLIPDGK |
| | RIIWDSRKGFIISNATYKEIGLLTCEATVNGHLYKTNYLTHRQTNTIIDVVLS |
| | PSHGIELSVGEKLVLNCTARTELNVGIDENWEYPSSKHQHKKLVNRDLKT |
| | QSGSEMKKFLSTLTIDGVTRSDQGLYTCAASSGLMTKKNSTFVRVHEKDK |
| | THTCPPCPAPELLGGPSVFLFPPKPKDTLMISRTPEVTCVVVDVSHEDPEVK |
| | FNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYP |
| | SDIAVEWESNGQPENNYKTTPPVLDSDGSFFLYSKLTVDKSRWQQGNVFS |
| | CSVMHEALHNHYTQKSLSLSPG |
| 92 | MVSYWDTGVLLCALLSCLLLTGSSSGSDTGRPFVEMYSEIPEIIHMTEGREL |
| | VIPCRVTSPNITVTLKKFPLDTLIPDGKRIIWDSRKGFIISNATYKEIGLLTCE |
| | ATVNGHLYKTNYLTHRQTNTIIDVVLSPSHGIELSVGEKLVLNCTARTELN |
| | VGIDFNWEYPSSKHQHKKLVNRDLKTQSGSEMKKFLSTLTIDGVTRSDQG |
| | LYTCAASSGLMTKKNSTFVRVHEKDKTHTCPPCPAPELLGGPSVFLFPPKP |
| | KDTLMISRTPEVTCVVVDVSHEDPEVKFNWYVDGVEVHNAKTKPREEQY |
| | NSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPG |

In some embodiments, the engineered polynucleotide comprises a viral vector such as an AAV vector comprising one or more expression cassettes for the one or more angiogenesis inhibitors. In some embodiments, the engineered polynucleotide comprises a vector. In some embodiments, the vector is a viral vector. In some embodiments, the engineered polynucleotide comprises an AAV vector. In some embodiments, the engineered polynucleotide comprises an AAV vector encoding an engineered AAV capsid. In some embodiments, the AAV vector comprises one or more expression cassettes for encoding an engineered polypeptide comprising: a peptide or a fusion protein comprising an antibody or fragment thereof operatively coupled to a peptide.

In some embodiments, the engineered polynucleotide is a vector. In some embodiments, the engineered polynucleotide is a viral vector comprising an AAV vector. In some embodiments, the engineered polynucleotide is an AAV vector comprising an AAV serotype comprising AAV1, AAV2, AAV3, AAV4, AAV5, AAV6, AAV7, AAV8, AAV9, AAV10, AAV11, AAV12, or any combination thereof. In some embodiments, the engineered polynucleotide is an AAV vector comprising the AAV2 serotype. In some embodiments, the AAV vector encodes an modified AAV capsid. In some embodiments, the engineered polynucleotide comprises a viral vector. In some embodiments, the viral vector comprises an AAV vector. In some embodiments, the AAV vector comprises an AAV serotype comprising AAV1, AAV2, AAV3, AAV4, AAV5, AAV6, AAV7, AAV8, AAV9, AAV10, AAV11, AAV12, or any combination thereof. In some embodiments, the AAV vector is an AAV2 vector. In some embodiments, the AAV vector encodes an engineered AAV capsid. In some embodiments, the engineered AAV capsid comprises an amino acid sequence of any one of SEQ ID NOs: 161-182 and SEQ ID NOs: 191-210. In some embodiments, the engineered AAV capsid comprises the amino acid sequence of SEQ ID NO: 169.

In some embodiments, the engineered polynucleotide comprises a nucleic acid sequence that is at least 75% identical, at least 80% identical, at least 85% identical, at least 90% identical, at least 95% identical, or at least 99% identical to of any one of SEQ ID NOs: 101-103 and 121-129. In some embodiments, the engineered polynucleotide comprises a nucleic acid sequence of any one of SEQ ID NOs: 101-103, and 121-129. In some embodiments, the engineered polynucleotide comprises a nucleic acid sequence of SEQ ID NOs: 101. In some embodiments, the engineered polynucleotide comprises a nucleic acid sequence of SEQ ID NOs: 102. In some embodiments, the engineered polynucleotide comprises a nucleic acid sequence of SEQ ID NOs: 103. In some embodiments, the engineered polynucleotide comprises a nucleic acid sequence of SEQ ID NOs: 121. In some embodiments, the engineered polynucleotide comprises a nucleic acid sequence of SEQ ID NOs: 122. In some embodiments, the engineered polynucleotide comprises a nucleic acid sequence of SEQ ID NOs: 123. In some embodiments, the engineered polynucleotide comprises a nucleic acid sequence of SEQ ID NOs: 124. In some embodiments, the engineered polynucleotide comprises a nucleic acid sequence of SEQ ID NOs: 125. In some embodiments, the engineered polynucleotide comprises a nucleic acid sequence of SEQ ID NOs: 126. In some embodiments, the engineered polynucleotide comprises a nucleic acid sequence of SEQ ID NOs: 127. In some embodiments, the engineered polynucleotide comprises a nucleic acid sequence of SEQ ID NOs: 128. In some embodiments, the engineered polynucleotide comprises a nucleic acid sequence of SEQ ID NOs: 129. In some embodiments, the engineered polynucleotide comprises a nucleic acid sequence of any one of SEQ ID NOs: 101-103, and 121-129.

In some embodiments, the engineered polynucleotide encodes a polypeptide comprising an amino acid sequence that is at least 75% identical, at least 80% identical, at least 85% identical, at least 90% identical, at least 95% identical, or at least 99% identical to any one of SEQ ID NO: 111-113 and 43-45. In some embodiments, the engineered polynucleotide encodes a polypeptide comprising an amino acid sequence that is any one of SEQ ID NO: 111-113 and 43-45.

In some embodiments, the engineered polynucleotide encodes a first angiogenesis inhibitor and a second angiogenesis inhibitor. In some embodiments, the first angiogenesis inhibitor comprises a complement 3 inhibitor, and the second angiogenesis inhibitor comprises a CNP. In some embodiments, the first angiogenesis inhibitor comprises the complement 3 inhibitor, and the second angiogenesis inhibitor comprises a CNP36. In some embodiments, the first angiogenesis inhibitor comprises the complement 3 inhibitor, and the second angiogenesis inhibitor comprises an Fc region and a CNP36. In some embodiments, the engineered AAV further encodes a third angiogenesis inhibitor. In some embodiments, the third angiogenesis inhibitor comprises an inhibitor of a membrane attack complex (MAC). In some embodiments, the inhibitor of the MAC comprises a CD59. In some embodiments, the engineered polynucleotide encodes a protease site flanked by the second angiogenesis inhibitor and the third angiogenesis inhibitor. In some embodiments, the first angiogenesis inhibitor comprises an inhibitor of a CD59, and the second angiogenesis inhibitor comprises a CNP. In some embodiments, the first angiogenesis inhibitor comprises the CD59, and the second angiogenesis inhibitor comprises a CNP36. In some embodiments, the first angiogenesis inhibitor comprises the CD59, and the second angiogenesis inhibitor comprises an Fc-CNP36. In some embodiments, the first angiogenesis inhibitor comprises the CD59, and the second angiogenesis inhibitor comprises a complement 3 inhibitor fused to an Fc-CNP36. In some embodiments, the first angiogenesis inhibitor comprises a complement 3 inhibitor, and the second angiogenesis inhibitor comprises an endostatin. In some embodiments, the engineered polynucleotide encodes an Fc region flanked by the first angiogenesis inhibitor and the second angiogenesis inhibitor. In some embodiments, the first angiogenesis inhibitor comprises a VEGF inhibitor, and the second angiogenesis inhibitor comprises a complement 3 inhibitor. In some embodiments, the first angiogenesis inhibitor comprises a complement 3 inhibitor, and the second angiogenesis inhibitor comprises an Fc-CNP36, wherein the engineered polynucleotide further encodes a third angiogenesis inhibitor comprising a CD59. In some embodiments, the engineered polynucleotide further encodes a protease site flanked by the second angiogenesis inhibitor and the third angiogenesis inhibitor. In some embodiments, the protease site comprises a Furin protease site. In some embodiments, the first angiogenesis inhibitor comprises a complement 3 inhibitor, and the second angiogenesis inhibitor comprises an Fc-CNP36, wherein the engineered polynucleotide further encodes a third angiogenesis inhibitor comprising a CD59. In some embodiments, the first angiogenesis inhibitor comprises a CD59, and the second angiogenesis inhibitor comprises a complement 3 inhibitor, wherein the engineered polynucleotide further encodes a third angiogenesis inhibitor comprising a Fc-CNP36. In some embodiments, the first angiogenesis inhibitor comprises a CD59, and the second angiogenesis inhibitor comprises an endostatin, wherein the engineered polynucleotide further encodes a third angiogenesis inhibitor comprising a complement 3 inhibitor. In some embodiments, the first angiogenesis inhibitor comprises a CD59, and the second angiogenesis inhibitor comprises a VEGF inhibitor, wherein the engineered polynucleotide further encodes a third angiogenesis inhibitor comprising a complement 3 inhibitor.

In some embodiments, the first angiogenesis inhibitor comprises a complement 3 inhibitor, and the second angiogenesis inhibitor comprises an endostatin, wherein the engineered polynucleotide further encodes a third angiogenesis inhibitor comprising a CD59. In some embodiments, the first angiogenesis inhibitor, the second angiogenesis inhibitor, and the third angiogenesis inhibitor is not a VEGF inhibitor. In some embodiments, the first angiogenesis inhibitor and the second angiogenesis inhibitor, upon administered to a subject, inhibits neovascularization in the subject. In some embodiments, the first angiogenesis inhibitor or the second angiogenesis inhibitor, upon administered to the subject, exhibits decreased inhibition of neovascularization in the subject compared to inhibition of neovascularization caused by a VEGF inhibitor.

In some embodiments, the engineered polynucleotide encodes sCD59-Fc4-CNP36. In some embodiments, the engineered polynucleotide encodes sCD59-C3i-Fc4-CNP36. In some embodiments, the engineered polynucleotide encodes C3i-Fc4-endostatin. In some embodiments, the engineered polynucleotide encodes Aflibercept (SEQ ID NO: 71)-linker-C3i. In some embodiments, the engineered polynucleotide encodes C3i-Fc4-CNP36-Furin-sCD59. sCD59-furin 2A-C3i-Fc4-CNP36. In some embodiments, the engineered polynucleotide encodes sCD59-furin 2A-endostatin-linker-C3i. In some embodiments, the engineered polynucleotide encodes sCD59-furin 2A-Aflibercept-linker-C3i. In some embodiments, the engineered polynucleotide encodes C3i-Fc4-CNP36-Furin-mCD59. In some embodiments, the engineered polynucleotide encodes C3i-Fc4-endostatin-Furin sCD59. In some embodiments, the engineered polynucleotide encodes C3i-Fc4-CNP36. In some embodiments, the engineered polynucleotide encodes Fc4-(G4S)4-CNP36. In some embodiments, the engineered polynucleotide encodes (DK)C3i-Fc4-CNP36. In some embodiments, the engineered polynucleotide encodes Fc4-C3i. In some embodiments, the engineered polynucleotide encodes Aflibercept-Fc1-C3i. In some embodiments, the engineered polynucleotide encodes C3i-Fc4-endostatin. In some embodiments, the engineered polynucleotide encodes (DK)C3i-Fc4-endostatin. In some embodiments, the engineered polynucleotide encodes Fc-C3i(T14A). In some embodiments, the engineered polynucleotide encodes Fc-C3i(+2Y). In some embodiments, the engineered polynucleotide encodes Fc-C3i(+2Y, T14A). In some embodiments, the engineered polynucleotide encodes Fc-C3i(−N15). In some embodiments, the engineered polynucleotide encodes Fc-C3i(T14A,−N15). In some embodiments, the engineered polynucleotide encodes Fc-C3i(+2Y,−N15). In some embodiments, the engineered polynucleotide encodes Fc-C3i(+2Y, T14A,−N15). In some embodiments, the engineered polynucleotide encodes Fc-C3i(N15Q). In some embodiments, the engineered polynucleotide encodes Fc-C3i(T14A, N15Q). In some embodiments, the engineered polynucleotide encodes Fc-C3i(+2Y, N15Q). In some embodiments, the engineered polynucleotide encodes Fc-C3i(+2Y, T14A, N15Q). In some embodiments, the engineered polynucleotide encodes Fc4-C3i(N15Q). In some embodiments, the engineered polynucleotide encodes C3i(N15Q)-Fc4-CNP36. In some embodiments, the engineered polynucleotide encodes C3i(N15Q)-Fc4-Endostatin. In some embodiments, the engineered polynucleotide encodes C3i(N15Q)-Fc4-C3i(N15Q). In some embodiments, the engineered polynucleotide encodes C3i(N15Q)-Fc4-Endostatin. In some embodiments, the engineered polynucleotide encodes sCD59. In some embodiments, the engineered polynucleotide encodes Vh(DK) sCD59. In some embodiments, the engineered polynucleotide encodes vsCD59-6×His. In some embodiments, the engineered polynucleotide encodes vsCD59 N18Q-6×His. In some embodiments, the engineered polynucleotide encodes vsCD59 Q34E-6×His. In some embodiments, the engineered polynucleotide encodes vsCD59 K38R-6×His. In some embodiments, the engineered polynucleotide encodes vsCD59 Q34E, K38R-6×His. In some embodiments, the engineered polynucleotide encodes vsCD59 N18Q, K38R-6×His. In some embodiments, the engineered polynucleotide encodes vsCD59 N18Q, Q33E, K38R-6×His.

In some embodiments, the engineered polynucleotide comprises a nucleic acid sequence that is at least 70% identical, that is at least 75% identical, at least 80% identical, at least 85% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical to of any one of SEQ ID NOs: 250-276, 304-311, and 320-324. In some embodiments, the engineered polynucleotide comprises a nucleic acid sequence of any one of SEQ ID NOs: 250-276, 304-311, and 320-324. In some embodiments, the engineered polynucleotide comprises a nucleic acid sequence of SEQ ID NOs: 250. In some embodiments, the engineered polynucleotide comprises a nucleic acid sequence of SEQ ID NOs: 251. In some embodiments, the engineered polynucleotide comprises a nucleic acid sequence of SEQ ID NOs: 252. In some embodiments, the engineered polynucleotide comprises a nucleic acid sequence of SEQ ID NOs: 253. In some embodiments, the engineered polynucleotide comprises a nucleic acid sequence of SEQ ID NOs: 254. In some embodiments, the engineered polynucleotide comprises a nucleic acid sequence of SEQ ID NOs: 255. In some embodiments, the engineered polynucleotide comprises a nucleic acid sequence of SEQ ID NOs: 256. In some embodiments, the engineered polynucleotide comprises a nucleic acid sequence of SEQ ID NOs: 257. In some embodiments, the engineered polynucleotide comprises a nucleic acid sequence of SEQ ID NOs: 258. In some embodiments, the engineered polynucleotide comprises a nucleic acid sequence of SEQ ID NOs: 259. In some embodiments, the engineered polynucleotide comprises a nucleic acid sequence of SEQ ID NOs: 260. In some embodiments, the engineered polynucleotide comprises a nucleic acid sequence of SEQ ID NOs: 261. In some embodiments, the engineered polynucleotide comprises a nucleic acid sequence of SEQ ID NOs: 262. In some embodiments, the engineered polynucleotide comprises a nucleic acid sequence of SEQ ID NOs: 263. In some embodiments, the engineered polynucleotide comprises a nucleic acid sequence of SEQ ID NOs: 264. In some embodiments, the engineered polynucleotide comprises a nucleic acid sequence of SEQ ID NOs: 265. In some embodiments, the engineered polynucleotide comprises a nucleic acid sequence of SEQ ID NOs: 266. In some embodiments, the engineered polynucleotide comprises a nucleic acid sequence of SEQ ID NOs: 267. In some embodiments, the engineered polynucleotide comprises a nucleic acid sequence of SEQ ID NOs: 268. In some embodiments, the engineered polynucleotide comprises a nucleic acid sequence of SEQ ID NOs: 269. In some embodiments, the engineered polynucleotide comprises a nucleic acid sequence of SEQ ID NOs: 270. In some embodiments, the engineered polynucleotide comprises a nucleic acid sequence of SEQ ID NOs: 271. In some embodiments, the engineered polynucleotide comprises a nucleic acid sequence of SEQ ID NOS: 272. In some embodiments, the engineered polynucleotide comprises a nucleic acid sequence of SEQ ID NOs: 273. In some embodiments, the engineered polynucleotide comprises a nucleic acid sequence of SEQ ID NOs: 274. In some embodiments, the engineered polynucleotide comprises a nucleic acid sequence of SEQ ID NOs: 275. In some embodiments, the engineered polynucleotide comprises a nucleic acid sequence of SEQ ID NOs: 276. In some embodiments, the engineered polynucleotide comprises a nucleic acid sequence of SEQ ID NOs: 304. In some embodiments, the engineered polynucleotide comprises a nucleic acid sequence of SEQ ID NOs: 305. In some embodiments, the engineered polynucleotide comprises a nucleic acid sequence of SEQ ID NOs: 306. In some embodiments, the engineered polynucleotide comprises a nucleic acid sequence of SEQ ID NOs: 307. In some embodiments, the engineered polynucleotide comprises a nucleic acid sequence of SEQ ID NOs: 308. In some embodiments, the engineered polynucleotide comprises a nucleic acid sequence of SEQ ID NOs: 309. In some embodiments, the engineered polynucleotide comprises a nucleic acid sequence of SEQ ID NOs: 310. In some embodiments, the engineered polynucleotide comprises a nucleic acid sequence of SEQ ID NOs: 311. In some embodiments, the engineered polynucleotide comprises a nucleic acid sequence of SEQ ID NOs: 320. In some embodiments, the engineered polynucleotide comprises a nucleic acid sequence of SEQ ID NOs: 321. In some embodiments, the engineered polynucleotide comprises a nucleic acid sequence of SEQ ID NOs: 322. In some embodiments, the engineered polynucleotide comprises a nucleic acid sequence of SEQ ID NOs: 323. In some embodiments, the engineered polynucleotide comprises a nucleic acid sequence of SEQ ID NOs: 324.

In some embodiments, the engineered polynucleotide encodes a polypeptide comprising an amino acid sequence that is at least 70% identical, that is at least 75% identical, at least 80%-identical, at least 85% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical to any one of SEQ ID NO: 277-303, 312-319, and 325-329. In some embodiments, the engineered polynucleotide encodes a polypeptide comprising an amino acid sequence that is any one of SEQ ID NO: 277-303, 312-319, and 325-329.

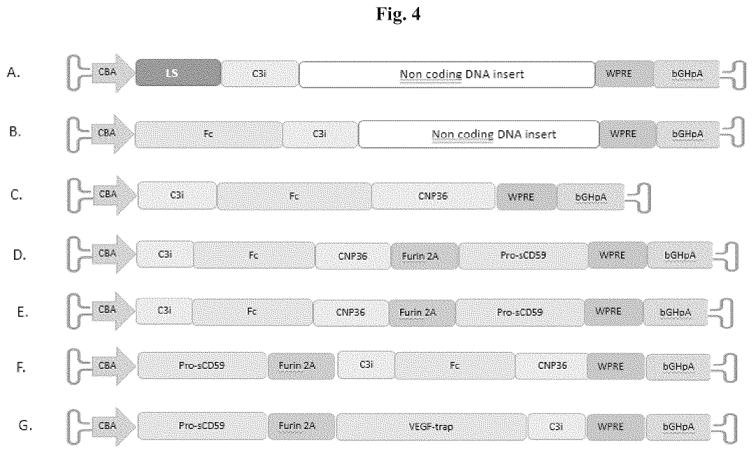

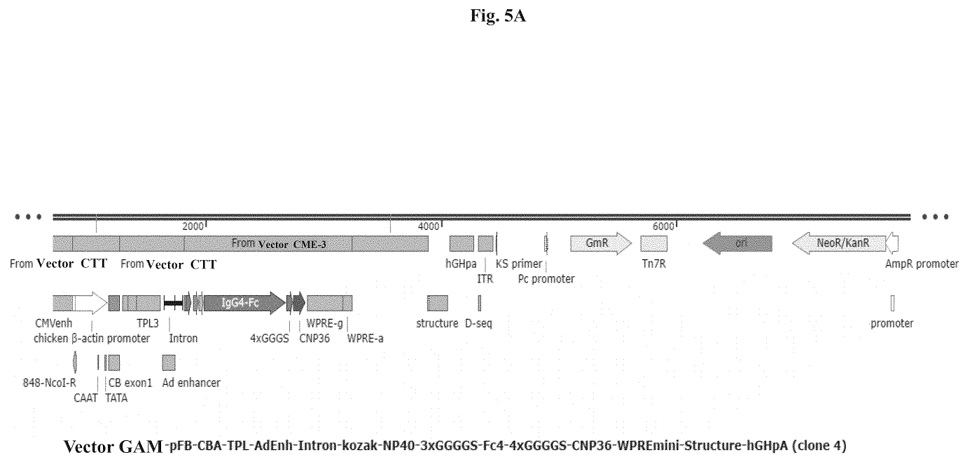

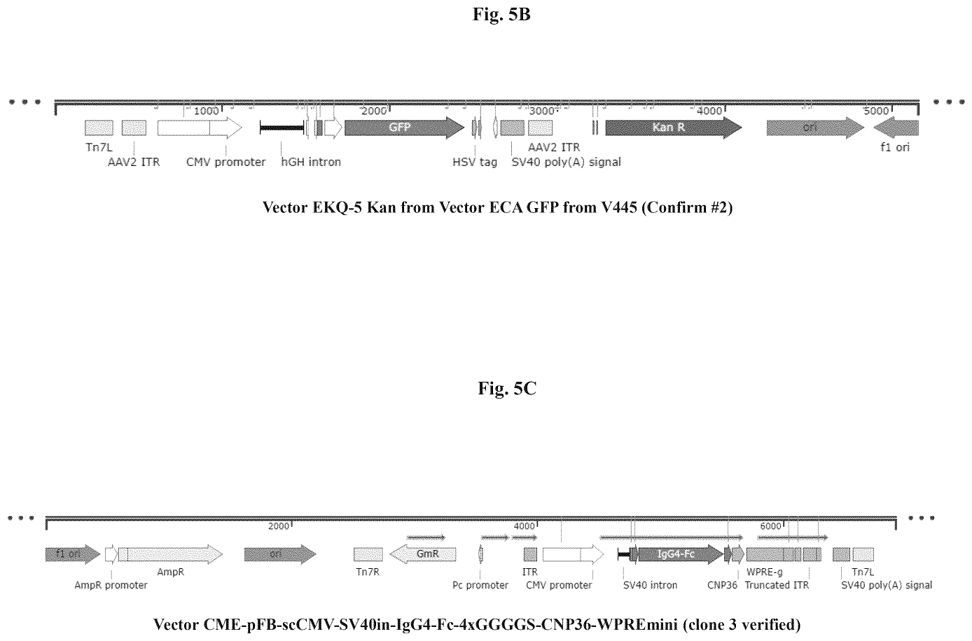

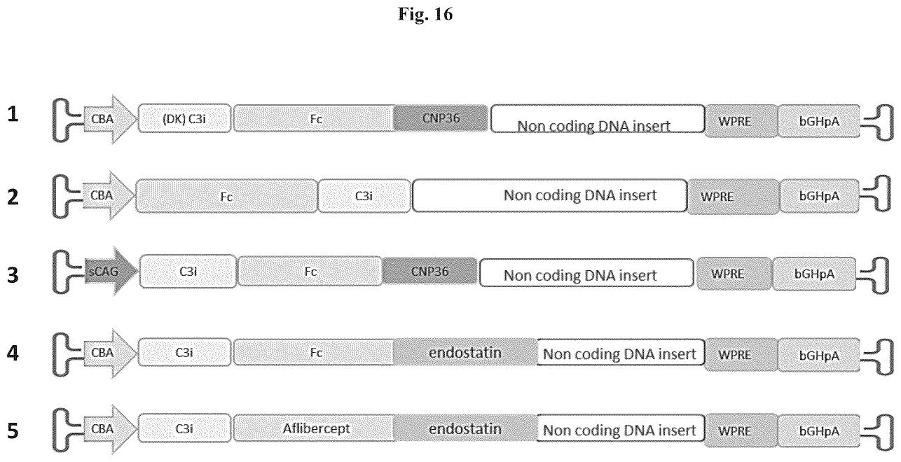

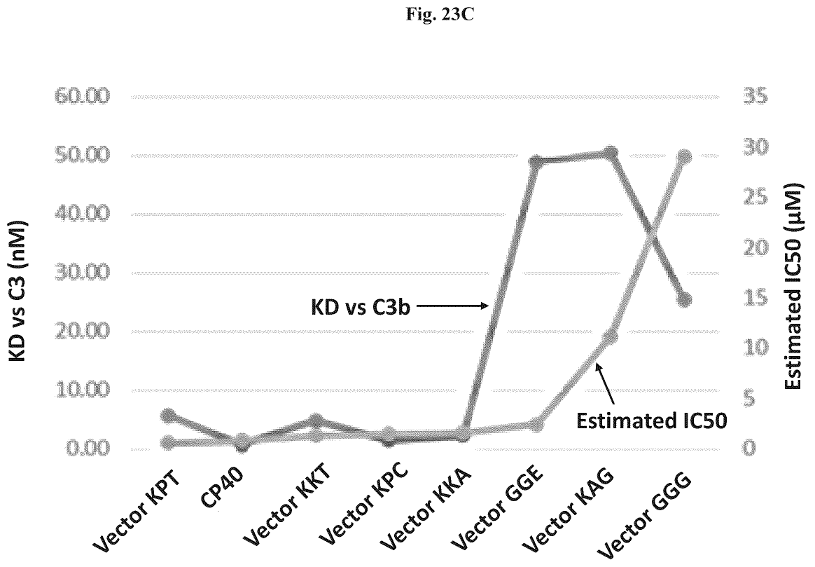

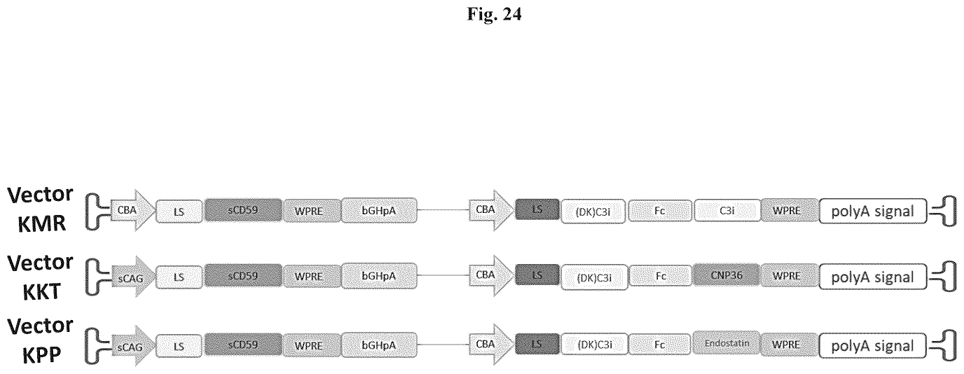

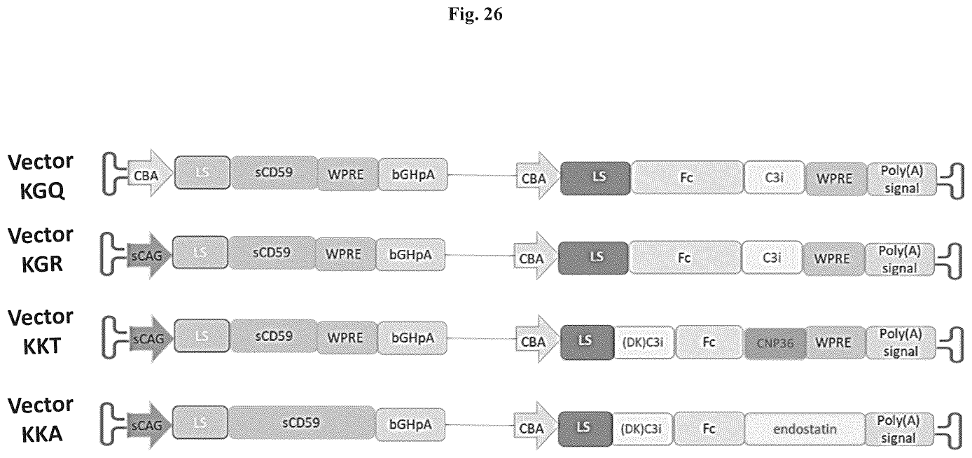

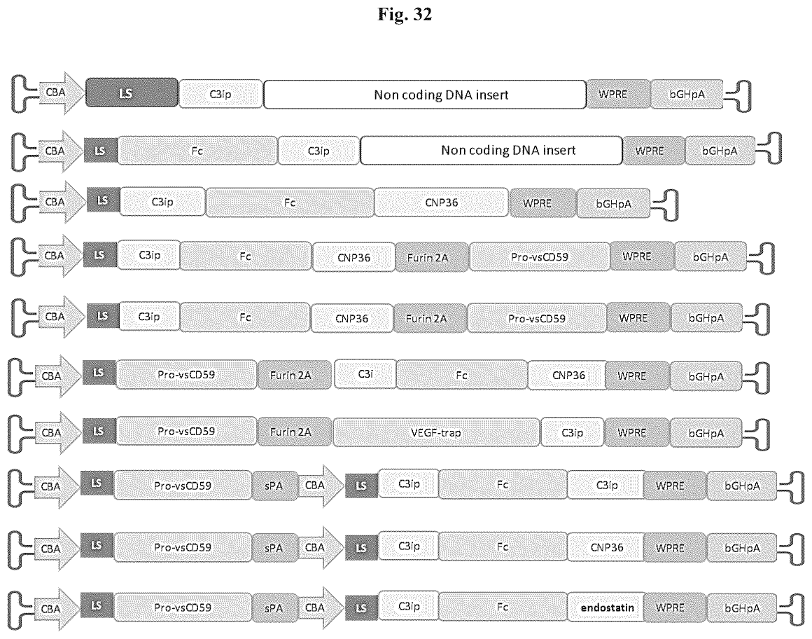

In some embodiments, the engineered polynucleotide encodes a sequence as shown in FIG. 1. In some embodiments, the engineered polynucleotide encodes a sequence as shown in FIGS. 2A-B. In some embodiments, the engineered polynucleotide encodes a sequence as shown in FIG. 3. In some embodiments, the engineered polynucleotide encodes a sequence as shown in FIG. 4. In some embodiments, the engineered polynucleotide encodes a sequence as shown in FIGS. 5A-C. In some embodiments, the engineered polynucleotide encodes a sequence as shown in FIG. 16. In some embodiments, the engineered polynucleotide encodes a sequence as shown in FIG. 24. In some embodiments, the engineered polynucleotide encodes a sequence as shown in FIG. 26. In some embodiments, the engineered polynucleotide encodes a sequence as shown in FIG. 32. In some cases, the engineered polynucleotide comprises additional features. Additional features can comprise sequences such as tags, signal peptides, intronic sequences, promoters, stuffer sequences, and the like. In some cases, the engineered polynucleotide encodes a signal peptide. A signal peptide is sometimes referred to as signal sequence, targeting signal, localization signal, localization sequence, transit peptide, leader sequence or leader peptide, is a short peptide present at the N-terminus of the majority of newly synthesized proteins that are destined toward the secretory pathway. These proteins include those that reside either inside certain organelles (the endoplasmic reticulum, Golgi or endosomes), secreted from the cell, or inserted into most cellular membranes. In some cases, nucleic acids provided herein can comprise signal peptides. A signal peptide can be of any length but typically from 15-30 amino acids long. A signal peptide can be from about: 10-15, 10-20, 10-30, 15-20, 15-25, 15-30, 20-30, or 25-30 amino acids long. Various signal peptides can be utilized and include but are not limited to: human antibody heavy chain (Vh), human antibody light chain (VI), and aflibercept.

In some cases, the engineered polynucleotide comprises an intronic sequence. An intron is any nucleotide sequence within a sequence that can be removed by RNA splicing during maturation of the final RNA product. In other words, introns are non-coding regions of an RNA transcript, or the DNA encoding it, that are eliminated by splicing before translation. While introns do not encode protein products, they are players in gene expression regulation. Some introns themselves encode functional RNAs through further processing after splicing to generate noncoding RNA molecules. Alternative splicing is widely used to generate multiple proteins from a single gene. Furthermore, some introns play essential roles in a wide range of gene expression regulatory functions such as nonsense-mediated decay and mRNA export. In an embodiment, an intronic sequence is included in a nucleic acid of the disclosure and can be selected from: hCMV intron A, adenovirus tripartite leader sequence intron, SV40 intron, hamster EF-1 alpha gene intron 1, intervening sequence intron, human growth hormone intron, and/or human beta globin intron. Any number of intronic sequences are contemplated. In an embodiment, the intronic sequence is SV40. In some cases, at least 1, at least 2, at least 3, at least 4, at least 5, at least 6, at least 7, at least 8, at least 9, or up to 10 intronic sequences can be included in a nucleic acid.

In an embodiment, the engineered polynucleotide comprises an additional feature including a promoter. Promoters are sequences of DNA to which proteins bind that initiate transcription of a single RNA from the DNA downstream of it. This RNA may encode a protein, or can have a function in and of itself, such as tRNA, mRNA, or rRNA. Promoters are located near the transcription start sites of genes, upstream on the DNA (towards the 5′ region of the sense strand). Promoters can be about 100-1000 base pairs long. Various promoters are contemplated and can be employed in the engineered polynucleotides of the disclosure. In an embodiment, a promoter is: a cytomegalovirus (CMV) promoter, an elongation factor 1 alpha (EF1α) promoter, a simian vacuolating virus (SV40) promoter, a phosphoglycerate kinase (PGK1) promoter, a ubiquitin C (Ubc) promoter, a human beta actin promoter, a CAG promoter, a Tetracycline response element (TRE) promoter, a UAS promoter, an Actin 5c (Ac5) promoter, a polyhedron promoter, a Ca2+/calmodulin-dependent protein kinase II (CaMKIIa) promoter, a GALI promoter, a GAL 10 promoter, a TEF1 promoter, a glyceraldehyde 3-phosphage dehydrogenase (GDS) promoter, an ADH1 promoter, a CaMV35S promoter, a Ubi promoter, a human polymerase III RNA (H1) promoter, a U6 promoter, a polyadenylated construct thereof, and any combination thereof. In some cases, the promoter is the CMV promoter.

Any of the provided the engineered polynucleotide can comprise viral vector sequences. A viral vector can be, without limitation, a lentivirus, a retrovirus, or an adeno-associated virus. A viral vector can be an adeno-associated viral (AAV) vector. In some cases, a viral vector is an adeno-associated viral vector. Many serotypes of AAV vectors are contemplated and include but are not limited to: AAV1, AAV2, AAV3, AAV4, AAV5, AAV6, AAV7, AAV8, AAV9, AAV10, AAV11, and/or AAV12. Based on these initial serotypes, AAV capsid of each serotype can be engineered to make them better suited for biological functions, tissue or cell selection. In some embodiments, an AAV vector is AAV2 and variants AAV2.N53 and AAV2.N54. Chimeric AAV vectors are also contemplated that may contain at least 2 AAV serotypes. In some cases, at least 3, at least 4, at least 5, at least 6, at least 7, or up to 8 different serotypes are combined in a chimeric AAV vector. In some cases, only a portion of the AAV is chimeric. For example, suitable portions can include the capsid, VP1, VP2, or VP3 domains and/or Rep. In some cases, at least one of VP1, VP2, and VP3 has at least one amino acid substitution compared to an otherwise comparable wild-type AAV capsid protein. In some cases, a mutation can occur in VP1 and VP2, in VP1 and VP3, in VP2 and VP3, or in VP1, VP2, and VP3. In some embodiments, at least one of VP1, VP2, and VP3 has from one to about 25 amino acid substitutions compared to wild-type AAV VP1, VP2, and VP3, e.g., from about one to about 5, from about 5 to about 10, from about 10 to about 15, from about 15 to about 20, or from about 20 to about 25 amino acid substitutions compared to wild-type AAV VP1, VP2, and VP3. In some cases, a VP can be removed. For example, in some embodiments a mutant AAV does not comprise at least one of VP1, VP2, or VP3.

In some cases, an AAV vector can be modified. For example, an AAV vector can comprise a modification such as an insertion, deletion, chemical alteration, or synthetic modification. In some cases, a single nucleotide is inserted into an AAV vector. In other cases, multiple nucleotides are inserted into a vector.

### Codon Optimization

In an embodiment, the engineered polynucleotide described herein comprises a modification that confers enhanced expression of the one or more angiogenesis inhibitors described herein. For example, the one or more angiogenesis inhibitors are derived from natural gene sequences and contain unmodified sequences that are not optimized for introduction and expression in target cells. In an embodiment, an isolated, engineered polynucleotide is codon optimized. Codon optimization can be specific for cell type-specific codon usage. Different organisms and cell types exhibit bias towards use of certain codons over others for the same amino acid. Some species are known to avoid certain codons almost entirely. Similarly, certain cell types are biased toward use of certain codons over others for the same amino acid. In an embodiment, a method of optimizing a codon of a engineered polynucleotide can comprise reassigning codon usage based on the frequencies of each codon's usage in a target cell. In some cases, a target cell can be of a certain tissue or organ. In some cases, a modification is performed to increase guanine and/or cytosine content.

In an embodiment, a engineered nucleic acid sequence can be modified to replace at least one codon with another codon coding for an identical amino acid. In some cases, a codon is modified within a coding region of a sequence. In some cases, a codon is modified within a non-coding region of a sequence. In some cases, a codon is modified within about 100, about 50, about 25, about 15, or about 5 bases from a termination codon. E-CAI can be utilized to estimate a value of a codon adaptation index.

Various modifications are contemplated herein. In some cases, codons can be interchanged. For example, a sequence can be modified to replace AGA with AGG. In other cases, CCC is replaced with CCT. In other cases, AGC is replaced with TCC. In other cases, CCC is replaced with CCG. Any of the non-limiting replacements provided in Table 16 can be applied to modify a nucleic acid. Any number of codons can be interchanged in a nucleic acid. In some cases, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 32, at least 34, at least 36, at least 38, at least 40, at least 42, at least 44, at least 46, at least 48, or up to 50 codons can be replaced. In an embodiment, a engineered polynucleotide comprises 3 codon modifications. In an embodiment, a engineered polynucleotide comprises 16 codon modifications. In an embodiment, a engineered polynucleotide comprises 3-5, 5-10, 5-15, 10-15, 10-20, 15-20, 1-20, 12-20, 12-25, 15-30, or 15-25 codon modifications. In an embodiment, a engineered polynucleotide comprises two codon modifications that are: AGA to AGG and at least one of: CCT to CCC, AGC to TCC, or CCC to CCG. In an embodiment, a engineered polynucleotide comprises three codon modifications that are: AGA to AGG and at least two of: CCT to CCC, AGC to TCC, or CCC to CCG. In an embodiment, a engineered polynucleotide comprises four codon modifications that are: AGA to AGG, CCT to CCC, AGC to TCC, and CCC to CCG. Additional modifications can comprise any of the codon modifications provided in Table 16 in combination with any of the above codons and/or any additional modifications possible from Table 16. In an embodiment, a nucleic acid is modified such that AGA is replaced with AGG and CCT is replaced with CCC. In an embodiment, a nucleic acid is modified such that AGA is replaced with AGG and AGC is replaced with TCC. In an embodiment, a nucleic acid is modified such that AGA is replaced with AGG and CCC is replaced with CCG.

TABLE 16
Non-limiting exemplary codons that can be interchanged
for modification of nucleic acids. Thymine can
be replaced with uracil in the below codons

| AA | Codons | AA | Codons |
|---|---|---|---|
| Ala | GCT, GCC, GCA, GCG | Leu | TTA, TTG, CTT, CTC, |
| | | | CTA, CTG |
| Arg | CGT, CGC, CGA, CGG, | Lys | AAA, AAG |
| | AGA, AGG | | |
| Asn | AAT, AAC | Met | ATG |
| Asp | GAT, GAC | Phe | TTT, TTC |
| Cys | TGT, TGC | Pro | CCT, CCC, CCA, CCU |
| Gln | CAA, CAG | Ser | TCT, TCC, TCA, TCG, |
| | | | AGT, AGC |
| Glu | GAA, GAG | Thr | ACT, ACC, ACA, ACG |
| Gly | GGT, GGC, GGA, GGG | Trp | TGG |
| His | CAT, CAC | Tyr | TAT, TAC |
| Ile | ATT, ATC, ATA | Val | GTT, GTC, GTA, GTG |
| Start | ATG | Stop | TAA, TGA, TAG |

In some embodiments, a engineered nucleic acid sequence can comprise a viral vector sequence. In some embodiments, a viral vector sequence can be a scAAV vector sequence. In some embodiments, a AAV vector sequence can be of serotype AAV1, AAV2, AAV3, AAV4, AAV5, AAV6, AAV7, AAV8, AAV9, AAV10, AAV11, AAV12, or any combination thereof. In some embodiments, an AAV vector sequence can be of the AAV2 serotype. In some embodiments, a viral vector sequence can comprise sequences of at least 2 AAV serotypes. In some embodiments, at least two serotypes can be selected from AAV1, AAV2, AAV3, AAV4, AAV5, AAV6, AAV8, AAV9, AAV11, and AAV12.

In some cases, a modification can also comprise a chemical modification. Modified nucleic acids can comprise modifications of their backbones, sugars, or nucleobases, and even novel bases or base pairs. Modified nucleic acids can have improved chemical and/or biological stability. Decoration with diverse chemical substituents (e.g., hydrophobic groups) can also yield improved properties and functionalities such as new structural motifs and enhanced target binding.

Exemplary chemical modification includes but are not limited to: 2′F, 2′-fluoro; 2′OMe, 2′-O-methyl; LNA, locked nucleic acid; FANA, 2′-fluoro arabinose nucleic acid; HNA, hexitol nucleic acid; 2′MOE, 2′-O-methoxyethyl; ribuloNA, (1′-3′)-β-L-ribulo nucleic acid; TNA, α-L-threose nucleic acid; tPhoNA, 3′-2′ phosphonomethyl-threosyl nucleic acid; dXNA, 2′-deoxyxylonucleic acid; PS, phosphorothioate; phNA, alkyl phosphonate nucleic acid; PNA, and peptide nucleic acid.

### Modified Capsid

Provided herein are modified adeno-associated virus (AAV) capsid-containing compositions and methods of using the same. A modified AAV capsid can comprise exogenous sequences as compared to an otherwise comparable unmodified AAV capsid. Exogenous sequences can refer to exogenous polypeptide sequences. AAV capsids can be modified to confer upon them, and any compositions and/or methods in which they are utilized, improved functionality thereby resulting in better therapeutics, particularly for ocular use.

The AAV wild-type (WT) genome contains at least three genes: rep, cap, and X. The X gene is located at the 3′ end of the genome (nucleotides 3929-4393 in AAV2) and seems to code for a protein with supportive function in genome replication. Significantly more information is available for rep and cap. The rep gene is located in the first half of the AAV WT genome and codes for a family of non-structural proteins (Rep proteins) required for viral transcription control and replication as well as packaging of viral genomes into the newly produced, pre-assembled capsids. The second half of the AAV genome contains the cap gene, which codes for the viral proteins (VPs) VP1, VP2, and VP3, and the assembly-activating protein (AAP). Transcription of all VPs, which are the capsid monomers, is controlled by a single promoter (p40 in case of AAV2) resulting in a single mRNA. Splicing (VP1) and an unusual translational start codon (VP2) are responsible for an approximately 10 times lower presence of VP1 and VP2 compared with VP3. When encoded by a single gene, AAV VPs share most of their amino acids. Specifically, the entire VP3 sequence is also contained within VP2 and VP1 (“common VP3 region”), and also VP2 and VP1 share approximately 65 amino acids (“common VP1/VP2 region”). Only VP1 contains a unique sequence at its N terminus (approximately 138 amino acids, VP1 unique). AAP was identified in 2010 as a 23 kDa protein encoded in an alternative cap ORF. It is used for stabilizing and transporting newly produced VP proteins from the cytoplasm into the cell nucleus. While AAV serotypes 1-3, 6-9, and rh10 failed to produce capsids in the absence of AAP, a low but detectable capsid production was reported for AAV4 and AAV5.

In an aspect, an AAV can comprise a modification. A modification can be of a rep, cap, and/or X coding polypeptide sequence of an AAV. In some cases, the modification can be of a cap polypeptide. A cap polypeptide can be modified in any one of the VP domains, for example VP1, VP2, and/or VP3. In some cases, VP1 is modified. In some cases, VP2 is modified. In some cases, VP3 is modified. In some aspects, two or all of the VP domains can be modified. In some cases, VP1 and VP2 are modified. In some cases, VP1 and VP3 are modified. Additionally, VP2 and VP3 can be modified or VP1, VP2, and VP3 are modified. Other combinations are contemplated, such as modifications in Rep and Cap, Cap and X, Rep and X, and/or Rep, Cap, and X. Any combination of domains can be modified such as any one of the aforementioned VP modifications in conjunction with a Rep and/or X modification. In some cases, Rep and VP1 and/or VP2 are modified. In some aspects, a subject Rep is modified. A rep modification can comprise a modification as provided herein and can be in at least one of Rep 78, Rep 68, Rep 52 or Rep 40. In some cases, a Rep is of a different AAV serotype than a subject capsid.

In some cases, a modification is of an AAV capsid. Capsids of AAV serotypes are assembled from 60 VP monomers with approximately 50 copies of VP3, 5 copies of VP2, and 5 copies of VP1. Topological prominent capsid surface structures are pores or “channel-like-structures” at each fivefold, depressions at each twofold, and three protrusions surrounding each threefold axis of symmetry. The pores allow exchange between the capsid interior and the outside. The depressions, more precisely the floor at each twofold axis, are the thinnest part of the viral capsid. The protrusions around the threefold axis harbor five of the nine so-called variable regions (VRs). Specifically, VR-IV, -V, and -VIII form loops (loop 1-4) at the top of the protrusions, while VR-VI and -VII are found at their base. VRs differ between serotypes and are responsible for serotype-specific variations in antibody and receptor binding. Because of their exposed positions and their function in receptor binding, VRs forming loops of the protrusions are ideal positions for capsid modifications aiming to re-direct or expand AAV tropism (cell surface targeting). While a re-directed tropism (vector re-targeting) combines ablation of natural receptor binding, for example by site-directed mutagenesis, with insertion of a ligand that mediates transduction through a novel non-natural AAV receptor, AAV vectors with tropism expansion gain the ability to transduce cells through an extra receptor while maintaining their natural receptor binding abilities.

In some aspects, a modification of an AAV capsid, can refer to an insertion of an exogenous polypeptide sequence. In other aspects, a modification can refer to a deletion in a polypeptide sequence. A modification can also refer to a modification of at least one amino acid residue, canonical or non-canonical, in a polypeptide sequence.

An insertion can comprise inserting at least 1 exogenous amino acid residue into a sequence coding an AAV capsid. The amino acid can refer to a canonical amino acid or a non-canonical amino acid. Any number of amino acid residues can be inserted. In some cases, an insertion site can be in the GH loop, or loop IV, of the AAV capsid protein, e.g., in a solvent-accessible portion of the GH loop, or loop IV, of the AAV capsid protein.

In some cases, a modification comprises insertion of an exogenous polypeptide sequence that comprises a sequence of Formula 1: X0-X1-X2-X1-X3-X1-X1-X4. In some cases, X0 is Valine (V), Isoleucine (I), Leucine (L), Phenylalanine (F), Tryptophan (W), Tyrosine (Y) or Methionine (M). In some cases, X1 is Alanine (A), Asparagine (N), Glutamine (Q), Serine(S), Threonine (T), Glutamic Acid (E), Aspartic Acid (D), Lysine (K), Arginine (R), or Histidine (H). In some cases, X2 is V, I, L, or M, where X3 is E, S, or Q. In some cases, X4 is K, R, E, or A. In some cases, Formula 1 further comprises X5. X5 can be Proline (P) or R.

In some cases, Formula 1 comprises: L-A-L-G-X3-X1-X1-X4 (SEQ ID NO: 232), L-K-L-G-X3-X1-X1-X4 (SEQ ID NO: 233), or V-K-L-G-X3-X1-X1-X4 (SEQ ID NO: 234). In some cases, Formula 1 comprises V-K-L-G-X3-X1-X1-X4 (SEQ ID NO: 235). In some cases, an exogenous polypeptide is V-K-L-G-X3-X1-T-X4 (SEQ ID NO: 236) and/or V-K-L-G-X3-X1-X1-K (SEQ ID NO: 237). In some cases, an exogenous polypeptide comprises L-A-L-G-X3-X1-X1-X4 (SEQ ID NO: 238). In some cases, an exogenous polypeptide comprises L-A-L-G-X3-X1-T-X4 (SEQ ID NO: 239) and/or L-A-L-G-X3-X1-S-X4 (SEQ ID NO: 240). In some cases, an exogenous polypeptide comprises: L-A-L-G-X3-X1-T-R (SEQ ID NO: 241), L-A-L-G-X3-X1-T-K (SEQ ID NO: 242), L-A-L-G-X3-X1-T-E (SEQ ID NO: 243), and/or L-A-L-G-X3-X1-T-A (SEQ ID NO: 244). In some cases, an exogenous polypeptide comprises L-A-L-G-X3-X1-S-K (SEQ ID NO: 246). In some cases, an exogenous polypeptide comprises L-K-L-G-X3-X1-X1-X4 (SEQ ID NO: 247). In some cases, an exogenous polypeptide comprises: L-K-L-G-X3-X1-T-X4 (SEQ ID NO: 248). In some cases, an exogenous polypeptide comprises: L-K-L-G-X3-X1-T-K (SEQ ID NO: 249).

In some cases, an exogenous polypeptide comprises a sequence of Formula 1. In some cases, a sequence of Formula I comprises a polypeptide sequence having at least 60%, 62%, 64%, 66%, 68%, 70%, 72%, 74%, 76%, 78%, 80%, 82%, 84%, 86%, 88%, 90%, 92%, 94%, 96%, 98%, or up to about 100% identity with a sequence of Table 17. In some cases, an exogenous polypeptide is one of Table 17 with 0-2 modifications to a residue.

In some cases, at least 2 of the exogenous polypeptides, such as those described by Formula 1, are inserted into a capsid sequence of an AAV provided herein. The at least 2 exogenous polypeptides can be inserted into the same location or at different locations. In an aspect, any one of the exogenous polypeptide sequences provided in Table 17 can be inserted into an unmodified AAV capsid sequence, such as those wildtype sequences provided in Table 18, to generate a modified AAV capsid.

TABLE 17
Exemplary exogenous polypeptide sequences that can be inserted into
AAV capsids (exemplary insertion sites are shown for AAV2 but
comparable locations of other AAV serotypes are also contemplated)

| SEQ ID | Modified Capsid Region | |
|---|---|---|
| NO | of AAV2 | Exogenous Polypeptide Sequence |
| 161 | 453 | LALGETTRPA |
| 162 | 587 | LALGETTKPA |
| 163 | 452 | ALGETTKP |
| 164 | 452 | ALGETTKP |
| 165 | 587 | LALGETTKPA |
| 166 | 587 | LKLGQTTKPK |
| 167 | 587 | LALGQTTKPK |
| 168 | 587 | LKLGQTTKPA |
| 169 | 587 | LALGQTTKPA |
| 170 | 453 | LALGQTTKPA |
| 171 | 587 | LALGQTTEPA |
| 172 | 588 | LALGQTTKPA |
| 173 | 585 | LALGQTTKPA |
| 174 | 587 | VALGQTTKPA |
| 175 | 586 | LALGESTARG |
| 176 | 586 | LALGETSKRA |
| 177 | 586 | LALGQSTKPA |
| 178 | 452 | LALGQTTKPA |
| 179 | 453 | LALGQTTKPA |
| 180 | 587 | LALGQTTKPA |
| 181 | 587 | LALGQTTKPALALGQTTKPA |
| 182 | 587 | VKLGQTTKPA |

| SEQ ID | |
|---|---|
| NO | Modified AAV2 Capsid Sequence |
| 191 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGLALGETTRPATTTQSRLQFSQAGAS |
| | DIRDQSRNWLPGPCYRQQRVSKTSADNNNSEYSWTGATK |
| | YHLNGRDSLVNPGPAMASHKDDEEKFFPQSGVLIFGKQGS |
| | EKTNVDIEKVMITDEEEIRTTNPVATEQYGSVSTNLQRGNR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 192 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGNLALGETTKPAR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 193 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSALGETTKPGTTTQSRLQFSQAGASDIR |
| | DQSRNWLPGPCYRQQRVSKTSADNNNSEYSWTGATKYHL |
| | NGRDSLVNPGPAMASHKDDEEKFFPQSGVLIFGKQGSEKT |
| | NVDIEKVMITDEEEIRTTNPVATEQYGSVSTNLQRGNRQAA |
| | TADVNTQGVLPGMVWQDRDVYLQGPIWAKIPHTDGHFHP |
| | SPLMGGFGLKHPPPQILIKNTPVPANPSTTFSAAKFASFITQY |
| | STGQVSVEIEWELQKENSKRWNPEIQYTSNYNKSVNVDFT |
| | VDTNGVYSEPRPIGTRYLTRNL |
| 194 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSALGETTKPGTTTQSRLQFSQAGASDIR |
| | DQSRNWLPGPCYRQQRVSKTSADNNNSEYSWTGATKYHL |
| | NGRDSLVNPGPAMASHKDDEEKFFPQSGVLIFGKQGSEKT |
| | NVDIEKVMITDEEEIRTTNPVATEQYGSVSTNLQRGNLALG |
| | ETTKPARQAATADVNTQGVLPGMVWQDRDVYLQGPIWA |
| | KIPHTDGHFHPSPLMGGFGLKHPPPQILIKNTPVPANPSTTFS |
| | AAKFASFITQYSTGQVSVEIEWELQKENSKRWNPEIQYTSN |
| | YNKSVNVDFTVDTNGVYSEPRPIGTRYLTRNL |
| 195 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGNLKLGQTTKPKR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 196 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGNLALGQTTKPKR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 197 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGNLKLGQTTKPAR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 198 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGNLALGQTTKPAR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 199 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGLALGQTTKPATTTQSRLQFSQAGAS |
| | DIRDQSRNWLPGPCYRQQRVSKTSADNNNSEYSWTGATK |
| | YHLNGRDSLVNPGPAMASHKDDEEKFFPQSGVLIFGKQGS |
| | EKTNVDIEKVMITDEEEIRTTNPVATEQYGSVSTNLQRGNR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 200 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGNLALGQTTEPAR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 201 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGNRLALGQTTKPA |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 202 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRLALGQTTKPAGNR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 203 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGNVALGQTTKPAR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 204 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGLALGESTARGNR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 205 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGLALGETSKRANR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 206 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGLALGQSTKPANR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 207 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSLALGQTTKPAGTTTQSRLQFSQAGAS |
| | DIRDQSRNWLPGPCYRQQRVSKTSADNNNSEYSWTGATK |
| | YHLNGRDSLVNPGPAMASHKDDEEKFFPQSGVLIFGKQGS |
| | EKTNVDIEKVMITDEEEIRTTNPVATEQYGSVSTNLQRGNR |
| | QAATADVNTQGVLPGMVWQDRDVYLQGPIWAKIPHTDG |
| | HFHPSPLMGGFGLKHPPPQILIKNTPVPANPSTTFSAAKFAS |
| | FITQYSTGQVSVEIEWELQKENSKRWNPEIQYTSNYNKSVN |
| | VDFTVDTNGVYSEPRPIGTRYLTRNL |
| 208 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGLALGQTTKPATTTQSRLQFSQAGAS |
| | DIRDQSRNWLPGPCYRQQRVSKTSADNNNSEYSWTGATK |
| | YHLNGRDSLVNPGPAMASHKDDEEKFFPQSGVLIFGKQGS |
| | EKTNVDIEKVMITDEEEIRTTNPVATEQYGSVSTNLQRGNL |
| | ALGQTTKPARQAATADVNTQGVLPGMVWQDRDVYLQGPI |
| | WAKIPHTDGHFHPSPLMGGFGLKHPPPQILIKNTPVPANPST |
| | TFSAAKFASFITQYSTGQVSVEIEWELQKENSKRWNPEIQY |
| | TSNYNKSVNVDFTVDTNGVYSEPRPIGTRYLTRNL |
| 209 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGNLALGQTTKPAL |
| | ALGQTTKPARQAATADVNTQGVLPGMVWQDRDVYLQGPI |
| | WAKIPHTDGHFHPSPLMGGFGLKHPPPQILIKNTPVPANPST |
| | TFSAAKFASFITQYSTGQVSVEIEWELQKENSKRWNPEIQY |
| | TSNYNKSVNVDFTVDTNGVYSEPRPIGTRYLTRNL |
| 210 | MAADGYLPDWLEDTLSEGIRQWWKLKPGPPPPKPAERHK |
| | DDSRGLVLPGYKYLGPFNGLDKGEPVNEADAAALEHDKA |
| | YDRQLDSGDNPYLKYNHADAEFQERLKEDTSFGGNLGRA |
| | VFQAKKRVLEPLGLVEEPVKTAPGKKRPVEHSPVEPDSSSG |
| | TGKAGQQPARKRLNFGQTGDADSVPDPQPLGQPPAAPSGL |
| | GTNTMATGSGAPMADNNEGADGVGNSSGNWHCDSTWM |
| | GDRVITTSTRTWALPTYNNHLYKQISSQSGASNDNHYFGY |
| | STPWGYFDFNRFHCHFSPRDWQRLINNNWGFRPKRLNFKL |
| | FNIQVKEVTQNDGTTTIANNLTSTVQVFTDSEYQLPYVLGS |
| | AHQGCLPPFPADVFMVPQYGYLTLNNGSQAVGRSSFYCLE |
| | YFPSQMLRTGNNFTFSYTFEDVPFHSSYAHSQSLDRLMNPL |
| | IDQYLYYLSRTNTPSGTTTQSRLQFSQAGASDIRDQSRNWL |
| | PGPCYRQQRVSKTSADNNNSEYSWTGATKYHLNGRDSLV |
| | NPGPAMASHKDDEEKFFPQSGVLIFGKQGSEKTNVDIEKV |
| | MITDEEEIRTTNPVATEQYGSVSTNLQRGNVAKGQTTKPA |
| | RQAATADVNTQGVLPGMVWQDRDVYLQGPIWAKIPHTD |
| | GHFHPSPLMGGFGLKHPPPQILIKNTPVPANPSTTFSAAKFA |
| | SFITQYSTGQVSVEIEWELQKENSKRWNPEIQYTSNYNKSV |
| | NVDFTVDTNGVYSEPRPIGTRYLTRNL |

TABLE 18
Exemplary wild type AAV capsid polypeptide sequences

| SEQ | AAV | |
|---|---|---|
| ID NO | Serotype | WT Polypeptide Sequence |
| 221 | AAV2 | MAADGYLPDWLEDTLSEGIRQWWKLKPGPPPPKPAERHKDDSR |
| | | GLVLPGYKYLGPFNGLDKGEPVNEADAAALEHDKAYDRQLDS |
| | | GDNPYLKYNHADAEFQERLKEDTSFGGNLGRAVFQAKKRVLEP |
| | | LGLVEEPVKTAPGKKRPVEHSPVEPDSSSGTGKAGQQPARKRLN |
| | | FGQTGDADSVPDPQPLGQPPAAPSGLGTNTMATGSGAPMADNN |
| | | EGADGVGNSSGNWHCDSTWMGDRVITTSTRTWALPTYNNHLY |
| | | KQISSQSGASNDNHYFGYSTPWGYFDFNRFHCHFSPRDWQRLIN |
| | | NNWGFRPKRLNFKLFNIQVKEVTQNDGTTTIANNLTSTVQVFTD |
| | | SEYQLPYVLGSAHQGCLPPFPADVFMVPQYGYLTLNNGSQAVG |
| | | RSSFYCLEYFPSQMLRTGNNFTFSYTFEDVPFHSSYAHSQSLDRL |
| | | MNPLIDQYLYYLSRTNTPSGTTTQSRLQFSQAGASDIRDQSRNW |
| | | LPGPCYRQQRVSKTSADNNNSEYSWTGATKYHLNGRDSLVNPG |
| | | PAMASHKDDEEKFFPQSGVLIFGKQGSEKTNVDIEKVMITDEEEI |
| | | RTTNPVATEQYGSVSTNLQRGNRQAATADVNTQGVLPGMVWQ |
| | | DRDVYLQGPIWAKIPHTDGHFHPSPLMGGFGLKHPPPQILIKNTP |
| | | VPANPSTTFSAAKFASFITQYSTGQVSVEIEWELQKENSKRWNPE |
| | | IQYTSNYNKSVNVDFTVDTNGVYSEPRPIGTRYLTRNL |
| 222 | AAV4 | MTDGYLPDWLEDNLSEGVREWWALQPGAPKPKANQQHQDNA |
| | | RGLVLPGYKYLGPGNGLDKGEPVNAADAAALEHDKAYDQQLK |
| | | AGDNPYLKYNHADAEFQQRLQGDTSFGGNLGRAVFQAKKRVL |
| | | EPLGLVEQAGETAPGKKRPLIESPQQPDSSTGIGKKGKQPAKKK |
| | | LVFEDETGAGDGPPEGSTSGAMSDDSEMRAAAGGAAVEGGQG |
| | | ADGVGNASGDWHCDSTWSEGHVTTTSTRTWVLPTYNNHLYKR |
| | | LGESLQSNTYNGFSTPWGYFDFNRFHCHFSPRDWQRLINNNWG |
| | | MRPKAMRVKIFNIQVKEVTTSNGETTVANNLTSTVQIFADSSYE |
| | | LPYVMDAGQEGSLPPFPNDVFMVPQYGYCGLVTGNTSQQQTDR |
| | | NAFYCLEYFPSQMLRTGNNFEITYSFEKVPFHSMYAHSQSLDRL |
| | | MNPLIDQYLWGLQSTTTGTTLNAGTATTNFTKLRPTNFSNFKKN |
| | | WLPGPSIKQQGFSKTANQNYKIPATGSDSLIKYETHSTLDGRWS |
| | | ALTPGPPMATAGPADSKFSNSQLIFAGPKQNGNTATVPGTLIFTS |
| | | EEELAATNATDTDMWGNLPGGDQSNSNLPTVDRLTALGAVPG |
| | | MVWQNRDIYYQGPIWAKIPHTDGHFHPSPLIGGFGLKHPPPQIFI |
| | | KNTPVPANPATTFSSTPVNSFITQYSTGQVSVQIDWEIQKERSKR |
| | | WNPEVQFTSNYGQQNSLLWAPDAAGKYTEPRAIGTRYLTHHL |
| 223 | AAV5 | MSFVDHPPDWLEEVGEGLREFLGLEAGPPKPKPNQQHQDQARG |
| | | LVLPGYNYLGPGNGLDRGEPVNRADEVAREHDISYNEQLEAGD |
| | | NPYLKYNHADAEFQEKLADDTSFGGNLGKAVFQAKKRVLEPFG |
| | | LVEEGAKTAPTGKRIDDHFPKRKKARTEEDSKPSTSSDAEAGPS |
| | | GSQQLQIPAQPASSLGADTMSAGGGGPLGDNNQGADGVGNAS |
| | | GDWHCDSTWMGDRVVTKSTRTWVLPSYNNHQYREIKSGSVDG |
| | | SNANAYFGYSTPWGYFDFNRFHSHWSPRDWQRLINNYWGFRP |
| | | RSLRVKIFNIQVKEVTVQDSTTTIANNLTSTVQVFTDDDYQLPYV |
| | | VGNGTEGCLPAFPPQVFTLPQYGYATLNRDNTENPTERSSFFCLE |
| | | YFPSKMLRTGNNFEFTYNFEEVPFHSSFAPSQNLFKLANPLVDQ |
| | | YLYRFVSTNNTGGVQFNKNLAGRYANTYKNWFPGPMGRTQG |
| | | WNLGSGVNRASVSAFATTNRMELEGASYQVPPQPNGMTNNLQ |
| | | GSNTYALENTMIFNSQPANPGTTATYLEGNMLITSESETQPVNR |
| | | VAYNVGGQMATNNQSSTTAPATGTYNLQEIVPGSVWMERDVY |
| | | LQGPIWAKIPETGAHFHPSPAMGGFGLKHPPPMMLIKNTPVPGNI |
| | | TSFSDVPVSSFITQYSTGQVTVEMEWELKKENSKRWNPEIQYTN |
| | | NYNDPQFVDFAPDSTGEYRTTRPIGTRYLTRPL |
| 224 | AAV6 | MAADGYLPDWLEDNLSEGIREWWDLKPGAPKPKANQQKQDD |
| | | GRGLVLPGYKYLGPFNGLDKGEPVNAADAAALEHDKAYDQQL |
| | | KAGDNPYLRYNHADAEFQERLQEDTSFGGNLGRAVFQAKKRV |
| | | LEPFGLVEEGAKTAPGKKRPVEQSPQEPDSSSGIGKTGQQPAKK |
| | | RLNFGQTGDSESVPDPQPLGEPPATPAAVGPTTMASGGGAPMA |
| | | DNNEGADGVGNASGNWHCDSTWLGDRVITTSTRTWALPTYNN |
| | | HLYKQISSASTGASNDNHYFGYSTPWGYFDFNRFHCHFSPRDW |
| | | QRLINNNWGFRPKRLNFKLFNIQVKEVTTNDGVTTIANNLTSTV |
| | | QVFSDSEYQLPYVLGSAHQGCLPPFPADVFMIPQYGYLTLNNGS |
| | | QAVGRSSFYCLEYFPSQMLRTGNNFTFSYTFEDVPFHSSYAHSQ |
| | | SLDRLMNPLIDQYLYYLNRTQNQSGSAQNKDLLFSRGSPAGMS |
| | | VQPKNWLPGPCYRQQRVSKTKTDNNNSNFTWTGASKYNLNGR |
| | | ESIINPGTAMASHKDDKDKFFPMSGVMIFGKESAGASNTALDNV |
| | | MITDEEEIKATNPVATERFGTVAVNLQSSSTDPATGDVHVMGAL |
| | | PGMVWQDRDVYLQGPIWAKIPHTDGHFHPSPLMGGFGLKHPPP |
| | | QILIKNTPVPANPPAEFSATKFASFITQYSTGQVSVEIEWELQKEN |
| | | SKRWNPEVQYTSNYAKSANVDFTVDNNGLYTEPRPIGTRYLTR |
| | | PL |
| 225 | AAV11 | MAADGYLPDWLEDNLSEGIREWWDLKPGAPKPKANQQKQDD |
| | | GRGLVLPGYKYLGPFNGLDKGEPVNAADAAALEHDKAYDQQL |
| | | KAGDNPYLRYNHADAEFQERLQEDTSFGGNLGRAVFQAKKRV |
| | | LEPLGLVEEGAKTAPGKKRPLESPQEPDSSSGIGKKGKQPARKR |
| | | LNFEEDTGAGDGPPEGSDTSAMSSDIEMRAAPGGNAVDAGQGS |
| | | DGVGNASGDWHCDSTWSEGKVTTTSTRTWVLPTYNNHLYLRL |
| | | GTTSSSNTYNGFSTPWGYFDFNRFHCHFSPRDWQRLINNNWGL |
| | | RPKAMRVKIFNIQVKEVTTSNGETTVANNLTSTVQIFADSSYELP |
| | | YVMDAGQEGSLPPFPNDVFMVPQYGYCGIVTGENQNQTDRNAF |
| | | YCLEYFPSQMLRTGNNFEMAYNFEKVPFHSMYAHSQSLDRLMN |
| | | PLLDQYLWHLQSTTSGETLNQGNAATTFGKIRSGDFAFYRKNW |
| | | LPGPCVKQQRFSKTASQNYKIPASGGNALLKYDTHYTLNNRWS |
| | | NIAPGPPMATAGPSDGDFSNAQLIFPGPSVTGNTTTSANNLLFTS |
| | | EEEIAATNPRDTDMFGQIADNNQNATTAPITGNVTAMGVLPGM |
| | | VWQNRDIYYQGPIWAKIPHADGHFHPSPLIGGFGLKHPPPQIFIK |
| | | NTPVPANPATTFTAARVDSFITQYSTGQVAVQIEWEIEKERSKR |
| | | WNPEVQFTSNYGNQSSMLWAPDTTGKYTEPRVIGSRYLTNHL |
| 226 | AAV12 | MAADGYLPDWLEDNLSEGIREWWALKPGAPQPKANQQHQDN |
| | | GRGLVLPGYKYLGPFNGLDKGEPVNEADAAALEHDKAYDKQL |
| | | EQGDNPYLKYNHADAEFQQRLATDTSFGGNLGRAVFQAKKRIL |
| | | EPLGLVEEGVKTAPGKKRPLEKTPNRPTNPDSGKAPAKKKQKD |
| | | GEPADSARRTLDFEDSGAGDGPPEGSSSGEMSHDAEMRAAPGG |
| | | NAVEAGQGADGVGNASGDWHCDSTWSEGRVTTTSTRTWVLPT |
| | | YNNHLYLRIGTTANSNTYNGFSTPWGYFDFNRFHCHFSPRDWQ |
| | | RLINNNWGLRPKSMRVKIFNIQVKEVTTSNGETTVANNLTSTVQ |
| | | IFADSTYELPYVMDAGQEGSFPPFPNDVFMVPQYGYCGVVTGK |
| | | NQNQTDRNAFYCLEYFPSQMLRTGNNFEVSYQFEKVPFHSMYA |
| | | HSQSLDRMMNPLLDQYLWHLQSTTTGNSLNQGTATTTYGKITT |
| | | GDFAYYRKNWLPGACIKQQKFSKNANQNYKIPASGGDALLKY |
| | | DTHTTLNGRWSNMAPGPPMATAGAGDSDFSNSQLIFAGPNPSG |
| | | NTTTSSNNLLFTSEEEIATTNPRDTDMFGQIADNNQNATTAPHIA |
| | | NLDAMGIVPGMVWQNRDIYYQGPIWAKVPHTDGHFHPSPLMG |
| | | GFGLKHPPPQIFIKNTPVPANPNTTFSAARINSFLTQYSTGQVAV |
| | | QIDWEIQKEHSKRWNPEVQFTSNYGTQNSMLWAPDNAGNYHE |
| | | LRAIGSRFLTHHL |

Similarly, a deletion can comprise deleting at least 1 amino acid residue in a sequence that codes for an AAV capsid. Any number of amino acids can be deleted. In some cases, at least, or at most: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or up to about 50 exogenous amino acid residues can be inserted and/or deleted in a polypeptide sequence that codes for an AAV capsid. In some cases, at least or at most: 1-5, 5-10, 10-15, 15-20, or combinations thereof of exogenous amino acid residues can be inserted and/or deleted in a polypeptide sequence that codes for an AAV capsid. In some cases, from about or up to about: 5 amino acids to about 11 amino acids are inserted in an insertion site in the GH loop or loop IV of the capsid protein relative to a corresponding unmodified AAV capsid protein. For example, the insertion site can be between amino acids 587 and 588 of AAV2, or the corresponding positions of the capsid subunit of another AAV serotype. It should be noted that the insertion site 587-588 is based on an AAV2 capsid protein. From about 5 amino acids to about 11 amino acids can be inserted in a corresponding site in an AAV serotype other than AAV2 (e.g., AAV5, AAV6, AAV8, AAV9, etc.).

In some embodiments, the insertion site is a single insertion site between two adjacent amino acids located between amino acids 570-614 of VP1 of any AAV serotype, e.g., the insertion site is between two adjacent amino acids located in amino acids 570-610, amino acids am-600, amino acids 570-575, amino acids 575-580, amino acids 580-585, amino acids 585-590, amino acids 590-600, or amino acids 600-614, of VP1 of any AAV serotype or variant. For example, the insertion site can be between amino acids 580 and 581, amino acids 581 and 582, amino acids 583 and 584, amino acids 584 and 585, amino acids 585 and 586, amino acids 586 and 587, amino acids 587 and 588, amino acids 588 and 589, or amino acids 589 and 590. The insertion site can be between amino acids 575 and 576, amino acids 576 and 577, amino acids 577 and 578, amino acids 578 and 579, or amino acids 579 and 580. The insertion site can be between amino acids 590 and 591, amino acids 591 and 592, amino acids 592 and 593, amino acids 593 and 594, amino acids 594 and 595, amino acids 595 and 596, amino acids 596 and 597, amino acids 597 and 598, amino acids 598 and 599, or amino acids 599 and 600.

In some aspects, an insertion site can be between amino acids 587 and 588 of AAV2, between amino acids 590 and 591 of AAV1, between amino acids 575 and 576 of AAV5, between amino acids 590 and 591 of AAV6, between amino acids 589 and 590 of AAV7, between amino acids 590 and 591 of AAV8, between amino acids 588 and 589 of AAV9, or between amino acids 588 and 589 of AAV10.

As another example, the insertion site can be between amino acids 450 and 460 of an AAV capsid protein, as shown in Table 18. For example, the insertion site can be at (e.g., immediately N-terminal to) amino acid 453 of AAV2, at amino acid 454 of AAV1, at amino acid 454 of AAV6, at amino acid 456 of AAV7, at amino acid 456 of AAV8, at amino acid 454 of AAV9, or at amino acid 456 of AAV10.

In some embodiments, a subject capsid protein includes a GH loop comprising an amino acid sequence having at least about 85%, at least about 90%, at least about 95%, at least about 98%, at least about 99%, or 100%, amino acid sequence identity to an amino acid sequence set forth in Table 18. Those skilled in the art would know, based on a comparison of the amino acid sequences of capsid proteins of various AAV serotypes, where an insertion site “corresponding to amino acids 587-588 of AAV2” would be in a capsid protein of any given AAV serotype.

In some cases, an exogenous polypeptide can have from 0 to 4 spacer amino acids (Y₁-Y₄) at the amino terminus and/or at the carboxyl terminus of any one of the exemplary polypeptides of Table 17 or Formula 1. Suitable spacer amino acids include, but are not limited to, leucine, alanine, glycine, and/or serine.

A modification of an AAV capsid can comprise a modification of at least one amino acid residue in a polypeptide sequence. In some cases, a modification can be made at any AAV capsid position, as described herein, and can include any number of modifications. In some cases, a modification can comprise a mutation. A mutation can comprise: a point mutation, missense mutation, nonsense mutation, deletion, duplication, frameshift, and/or repeat expansion.

In an aspect, an amino acid can be a non-polar, aliphatic residue such as glycine, alanine, valine, leucine, methionine, isoleucine, or proline. In an aspect, an amino acid residue is aromatic and is phenylalanine, tyrosine, or tryptophan. In an aspect, an amino acid residue is polar, non-charged and is serine, threonine, cysteine, asparagine, or glutamine. In an aspect, an amino acid is positively charged and is lysine, arginine, or histidine. In an aspect, an amino acid is negatively charged and is aspartate or glutamate.

In some cases, a mutation is a point mutation. A point mutation comprises a change from a charged amino acid residue to a polar or non-polar amino acid residue. In some cases, the charged amino acid is positively charged. In some cases, the charged amino acid is negatively charged.

A point mutation can be a conservative mutation. Non-limiting examples of conservative mutations comprise: a nonpolar aliphatic amino acid to a nonpolar aliphatic amino acid, a polar amino acid to a polar amino acid, a positively charged amino acid to a positively charged amino acid, a negatively charged amino acid to a negatively charged amino acid, and an aromatic amino acid to an aromatic amino acid. For example, 20 naturally occurring amino acids can share similar characteristics. Aliphatic amino acids can be: glycine, alanine, valine, leucine, or isoleucine. Hydroxyl or sulfur/selenium-containing amino acids can be: Serine, cysteine, selenocysteine, threonine, or methionine. A cyclic amino acid can be proline. An aromatic amino acid can be phenylalanine, tyrosine, or tryptophan. A basic amino acid can be histidine, lysine, and arginine. An acidic amino acid can be aspartate, glutamate, asparagine, or glutamine. A conservative mutation can be, serine to glycine, serine to alanine, serine to serine, serine to threonine, serine to proline. A conservative mutation can be arginine to asparagine, arginine to lysine, arginine to glutamine, arginine to arginine, arginine to histidine. A conservative mutation can be Leucine to phenylalanine, leucine to isoleucine, leucine to valine, leucine to leucine, leucine to methionine. A conservative mutation can be proline to glycine, proline to alanine, proline to serine, proline to threonine, proline to proline. A conservative mutation can be threonine to glycine, threonine to alanine, threonine to serine, threonine to threonine, threonine to proline. A conservative mutation can be alanine to glycine, alanine to threonine, alanine to proline, alanine to alanine, alanine to serine. A conservative mutation can be valine to methionine, valine to phenylalanine, valine to isoleucine, valine to leucine, valine to valine. A conservative mutation can be glycine to alanine, glycine to threonine, glycine to proline, glycine to serine, glycine to glycine. A conservative mutation can be Isoleucine to phenylalanine, isoleucine to isoleucine, isoleucine to valine, isoleucine to leucine, isoleucine to methionine. A conservative mutation can be phenylalanine to tryptophan, phenylalanine to phenylalanine, phenylalanine to tyrosine. A conservative mutation can be tyrosine to tryptophan, tyrosine to phenylalanine, tyrosine to tyrosine. A conservative mutation can be cysteine to serine, cysteine to threonine, cysteine to cysteine. A conservative mutation can be histidine to asparagine, histidine to lysine, histidine to glutamine, histidine to arginine, histidine to histidine. A conservative mutation can be glutamine to glutamic acid, glutamine to asparagine, glutamine to aspartic acid, glutamine to glutamine. A conservative mutation can be asparagine to glutamic acid, asparagine to asparagine, asparagine to aspartic acid, asparagine to glutamine. A conservative mutation can be lysine to asparagine, lysine to lysine, lysine to glutamine, lysine to arginine, lysine to histidine. A conservative mutation can be aspartic acid to glutamic acid, aspartic acid to asparagine, aspartic acid to aspartic acid, aspartic acid to glutamine. A conservative mutation can be glutamine to glutamine, glutamine to asparagine, glutamine to aspartic acid, glutamine to glutamine. A conservative mutation can be methionine to phenylalanine, methionine to isoleucine, methionine to valine, methionine to leucine, methionine to methionine. A conservative mutation can be tryptophan to tryptophan, tryptophan to phenylalanine, tryptophan to tyrosine.

Non-limiting examples of additional amino acid mutations can be: A to R, A to N, A to D, A to C, A to Q, A to E, A to G, A to H, A to I, A to L, A to K, A to M, A to F, A to P, A to S, A to T, A to W, A to Y, A to V, R to N, R to D, R to C, R to Q, R to E, R to G, R to H, R to I, R to L, R to K, R to M, R to F, R to P, R to S, R to T, R to W, R to Y, R to V, N to D, N to C, N to Q, N to E, N to G, N to H, N to I, N to L, N to K, N to M, N to F, N to P, N to S, N to T, N to W, N to Y, N to V, D to C, D to Q, D to E, D to G, D to H, D to I, D to L, D to K, D to M, D to F, D to P, D to S, D to T, D to W, D to Y, D to V, C to Q, C to E, C to G, C to H, C to I, C to L, C to K, C to M, C to F, C to P, C to S, C to T, C to W, C to Y, C to V, Q to E, Q to G, Q to H, Q to I, Q to L, Q to K, Q to M, Q to F, Q to P, Q to S, Q to T, Q to W, Q to Y, Q to V, E to G, E to H, E to I, E to L, E to K, E to M, E to F, E to P, E to S, E to T, E to W, E to Y, E to V, G to H, G to I, G to L, G to K, G to M, G to F, G to P, G to S, G to T, G to W, G to Y, G to V, H to I, H to L, H to K, H to M, H to F, H to P, H to S, H to T, H to W, H to Y, H to V, I to L, I to K, I to M, I to F, I to P, I to S, I to T, I to W, I to Y, I to V, L to K, L to M, L to F, L to P, L to S, L to T, L to W, L to Y, L to V, K to M, K to F, K to P, K to S, K to T, K to W, K to Y, K to V, M to F, M to P, M to S, M to T, M to W, M to Y, M to V, F to P, F to S, F to T, F to W, F to Y, F to V, P to S, P to T, P to W, P to Y, P to V, S to T, S to W, S to Y, S to V, T to W, T to Y, T to V, W to Y, W to V, Y to V, and any of the previously described mutations in reverse.

Any one of the aforementioned modifications, insertions, deletions, and/or mutations, can be made at any residue in an AAV sequence. The sequence may be a capsid sequence. In other cases, the sequence may not be a capsid sequence but rather a Rep and/or X sequence. The sequence may be in a VP1, VP2, and/or VP3 as previously described. In some cases, the sequence modification is of a loop of a capsid sequence, such as loop 3 and/or loop 4. In some cases, the modification is of a residue of a sequence in Table 18.

In some cases, a modification, such as insertion, deletion, and/or mutation is of a residue of a capsid polypeptide sequence in Table 18. In some cases, a modification is from 1-100, 100-200, 200-300, 300-400, 400-500, 500-600, 600-700, 700-800, or combinations thereof. In some cases, a modification is in a residue at position 200-300, 300-400, 400-500, 500-600 or combinations thereof. In some cases, a modification is in a residue at position 300-500 or combinations thereof. In an aspect, an insertion site is in the GH loop, or loop IV, of the AAV capsid protein, e.g., in a solvent-accessible portion of the GH loop, or loop IV, of the AAV capsid protein. For example, the insertion site is within amino acids 570-611 of AAV2, within amino acids 571-612 of AAV1, within amino acids 560-601 of AAV5, within amino acids 571 to 612 of AAV6, within amino acids 572 to 613 of AAV7, within amino acids 573 to 614 of AAV8, within amino acids 571 to 612 of AAV9, or within amino acids 573 to 614 of AAV10.

For example, the insertion site can be between amino acids 587 and 588 of AAV2, between amino acids 590 and 591 of AAV1, between amino acids 575 and 576 of AAV5, between amino acids 590 and 591 of AAV6, between amino acids 589 and 590 of AAV7, between amino acids 590 and 591 of AAV8, between amino acids 588 and 589 of AAV9, or between amino acids 589 and 590 of AAV10. In some cases, a modification is at position 452, 453, 466, 467, 468, 471, 585, 586, 587, and/or 588 of AAV2. In some cases, a modification is at position 452 or 453 of AAV2. In some cases, a modification is at position 587 or 588 of AAV2. In some cases, a modification is an insertion at position 452, 453, 466, 467, 468, 471, 585, 586, 587, and/or 588 of any one of SEQ ID NOs: 221-226. In some cases, a modification is an insertion at position 452, 453, 466, 467, 468, 471, 585, 586, 587, and/or 588 of SEQ ID NO: 221. In some cases, a modification is a mutation and the mutation is R585A or R588A of any one of SEQ ID NOs: 221-226. In some cases, a modification is a mutation and the mutation is R585A or R588A of SEQ ID NO: 221.

In some embodiments, a subject modified AAV capsid does not include any other amino acid modifications mutations, substitutions, insertions, or deletions, other than an insertion of from about 5 amino acids to about 11 amino acids in a loop (loop 3 and/or 4) relative to a corresponding unmodified AAV capsid protein. In other embodiments, a subject variant AAV capsid includes from 1 to about 25 amino acid insertions, deletions, or substitutions, compared to an unmodified AAV capsid protein, in addition to an insertion of from about 5 amino acids to about 11 amino acids in the loop 3 and/or loop 4 relative to an unmodified AAV capsid protein. In an embodiment, a subject AAV virion capsid does not include any other amino acid substitutions, insertions, or deletions, other than an insertion of from about 7 amino acids to about 10 amino acids in a GH loop or loop IV relative to a corresponding parental AAV capsid protein. In other embodiments, a subject AAV virion capsid includes from 1 to about 25 amino acid insertions, deletions, or substitutions, compared to the parental AAV capsid protein, in addition to an insertion of from about 7 amino acids to about 10 amino acids in the GH loop or loop IV relative to a corresponding parental AAV capsid protein. For example, in some embodiments, a subject AAV virion capsid includes from 1 to about 5, from about 5 to about 10, from about 10 to about 15, from about 15 to about 20, or from about 20 to about 25 amino acid insertions, deletions, or substitutions, compared to the parental AAV capsid protein, in addition to an insertion of from about 7 amino acids to about 10 amino acids in the GH loop or loop IV relative to a corresponding parental AAV capsid protein.

In some cases, a chimeric AAV capsid is provided herein. A chimeric capsid comprises a polypeptide sequence from at least 2 AAV serotypes. A chimeric capsid can comprise a mix of sequences selected from serotypes AAV1, AAV2, AAV3, AAV4, AAV5, AAV6, AAV7, AAV8, AAV9, AAV10, AAV11, and/or AAV12. In some cases, the chimeric serotypes are different between VP1, VP2, and/or VP3. In some cases, a chimeric capsid comprises sequences from at least 2 serotypes selected from: AAV4 and AAV6, AAV5 and AAV6, AAV11 and AAV6, AAV12 and AAV6, and any combination thereof. In some cases, a first AAV serotype can be AAV4 and a second serotype can be AAV6. In some cases, a first AAV serotype and a second AAV serotype of a chimeric AAV vector can be AAV11 and AAV6. In some cases, a first AAV serotype and a second AAV serotype of a chimeric AAV vector can be AAV12 and AAV6. In some cases, a chimeric capsid comprises sequences from: AAV2 and AAV5 or AAV2 and AAV6. In some cases, a chimeric capsid comprises sequences from: AAV2 and AAV5, AAV2 and AAV6, AAV2 and AAV8, AAV2 and AAV9, AAV2 and AAV1, and AAV2 and AAV12.

The modifications to an AAV provided herein can confer enhanced activity to the modified AAV as compared to an otherwise unmodified or wildtype AAV. Modifications provided herein can improve cell transduction, tropism, and/or reduce immunogenicity associated with the capsid.

In some cases, a modification provided herein enhances cellular transduction. Cellular transduction can refer to the ability of an AAV to infect a cell (in vivo or in vitro) and/or deliver a transgene into the cell.

In some cases, a modification provided herein enhances tropism. Enhanced tropism refers to gaining the ability to transduce cells through an extra receptor, as compared to an otherwise unmodified AAV. In some aspects, enhanced tropism can improve infectivity of an ocular cell, thereby improving gene therapy by way utilization of the modified AAV. In some cases, a modification provided herein can improve tropism to an ocular cell selected from: bipolar, retinal ganglion, horizontal, amacrine, epithelial, retinal pigment, photoreceptor, or any combination thereof. In some cases, a modification improves tropism to a retinal cell.

Also provided herein are AAV vectors. AAV vectors comprise: inverted terminal repeats (ITRs), Rep, Cap, AAP, and X sequences. Typically, the AAV viral genome is flanked by the ITRs, which serve as packaging signal and origin of replication. The rep gene encodes a family of multifunctional proteins (Rep proteins) responsible for controlling viral transcription, replication, packaging, and integration in AAVS1. For AAV2, four Rep proteins are described. Expression of Rep78 and Rep68 is controlled by the AAV2-specific p5 promoter, while p19 controls expression of the smaller Rep proteins (Rep52 and Rep40). Rep68 and Rep40 are splice variants of Rep78 and Rep52, respectively. Numbers indicate the molecular weight. Expression of AAP and the viral capsid proteins VP1 (90 kDa), VP2 (72 kDa), and VP3 (60 kDa), all encoded in the cap gene, is controlled by the p40 promoter. The X gene is located at the 3′ end of the genome within a region shared with the cap gene and possesses its own promoter (p81). While the X protein seems to enhance viral replication, AAP is essential for capsid assembly. The three different VPs contribute in a 1 (VP1): 1 (VP2): 10 (VP3) ratio to the icosahedral AAV2 capsid.

A modified capsid protein disclosed herein can be isolated, e.g., purified. In some embodiments, a modified capsid disclosed herein is included in an AAV vector or an AAV virion (for example recombinant AAV virion, rAAV, or an AAV viral particle). In other embodiments, such modified AAV vectors and/or AAV variant virions are used in an in vivo or ex vivo method of treating ocular disease in a primate retina, for example human retina.

Provided herein are also vectors that comprise modified AAV capsids. Any one of the previously described modifications can be encompassed in a vector provided herein. In some cases, an AAV vector comprises a modified capsid that comprises an exogenous sequence in at least two loops of a VP domain as compared to an otherwise comparable AAV capsid sequence that lacks the exogenous sequence. In some aspects, vectors provided herein can further comprise a transgene sequence.

### Engineered Polypeptide

Described herein, in some aspects, is an engineered polypeptide. In some embodiments, the engineered polypeptide is encoded by an engineered polynucleotide described herein. In some embodiments, the engineered polypeptide comprises a first angiogenesis inhibitor and a second angiogenesis inhibitor described herein. In some embodiments, the engineered polypeptide comprises a third angiogenesis inhibitor. In some embodiments, the engineered polypeptide comprises two or more angiogenesis inhibitors, where the two or more angiogenesis inhibitors are covalently connected by an antibody (e.g., an Fc region described herein) or a linker.

In some embodiments, the engineered polypeptide comprises complement 3 inhibitor and at least one additional angiogenesis inhibitor. In some embodiments, the complement 3 inhibitor (C3i) comprises an amino acid sequence that is at least 75% identical, at least 80% identical, at least 85% identical, at least 90% identical, at least 95% identical, or at least 99% identical to any one of SEQ ID NOs: 1-15. In some embodiments, the complement 3 inhibitor (C3i) comprises an amino acid sequence that is any one of SEQ ID NOs: 1-15. In some embodiments, the complement 3 inhibitor (C3i) comprises an amino acid sequence that is at 8 contiguous amino acids, at least 10 contiguous amino acids, or at least 12 contiguous amino acids of any one of SEQ ID NOs: 1-15.

In some embodiments, the engineered polypeptide comprises a natriuretic peptide and at least one additional angiogenesis inhibitor. In some embodiments, the natriuretic peptide is a CNP. In some embodiments, the CNP is covalently connected to a complement 3 inhibitor. In some embodiments, the CNP is covalently connected to a complement 3 inhibitor by a linker. In some embodiments, the natriuretic peptide or the natriuretic peptide fusion protein comprises an amino acid sequence that is at least 75% identical, at least 80% identical, at least 85% identical, at least 90% identical, at least 95% identical, or at least 99% identical to any one of SEQ ID NOs: 61-72. In some embodiments, the natriuretic peptide or the natriuretic peptide fusion protein comprises an amino acid sequence that is any one of SEQ ID NOs: 61-72.

In some embodiments, the engineered polypeptide comprises an inhibitor of a membrane attack complex (MAC) and at least one additional angiogenesis inhibitor. In some embodiments, the inhibitor of the MAC comprises CD59. In some embodiments, the CD59 comprises an amino acid sequence that is at least 75% identical, at least 80% identical, at least 85% identical, at least 90% identical, at least 95% identical, or at least 99% identical to any one of SEQ ID NOs: 41-45. In some embodiments, the CD59 comprises an amino acid sequence that is at least 75% identical, at least 80% identical, at least 85% identical, at least 90% identical, at least 95% identical, or at least 99% identical to any one of SEQ ID NOs: 312-319. In some embodiments, the CD59 comprises an amino acid sequence that is at least 75% identical, at least 80% identical, at least 85% identical, at least 90% identical, at least 95% identical, or at least 99% identical to any one of SEQ ID NOs: 325-329. In some embodiments, the CD59 comprises an amino acid sequence that is any one of SEQ ID NOs: 41-45. In some embodiments, the CD59 comprises an amino acid sequence that is any one of SEQ ID NOs: 312-319. In some embodiments, the CD59 comprises an amino acid sequence that is any one of SEQ ID NOs: 325-329.

In some embodiments, the engineered polypeptide comprises a collagen or fragment thereof. In some embodiments, the collagen or fragment thereof comprises an endostatin or fragment thereof. In some embodiments, the endostatin or fragment thereof comprises an amino acid sequence that is at least 75% identical, at least 80% identical, at least 85% identical, at least 90% identical, at least 95% identical, or at least 99% identical to SEQ ID NO: 51. In some embodiments, the endostatin or fragment thereof comprises an amino acid sequence that is SEQ ID NO: 51.

In some embodiments, the engineered polypeptide comprises an VEGF inhibitor and at least one additional angiogenesis inhibitor. In some embodiments, the VEGF antibody comprises a polypeptide sequence that is at least 70%, at least 75%, at least 80%, at least 85%, at least 90%, at least 95%, at least 99%, or more identical to any one of SEQ ID NOs: 81-87 or SEQ ID NOs: 88-92, or a combination thereof, or a fragment thereof. In some embodiments, the VEGF antibody comprises a polypeptide sequence that is any one of SEQ ID NOs: 81-87 or SEQ ID NOs: 88-92, or a combination thereof, or a fragment thereof.

In some embodiments, the engineered polypeptide comprises a complement 3 inhibitor, a fragment crystallizable (Fc) region, and a natriuretic peptide. In some embodiments, the engineered polypeptide comprises a complement 3 inhibitor and a natriuretic peptide. In some embodiments, the natriuretic peptide comprises a C-type natriuretic peptide (CNP). In some embodiments, the natriuretic peptide is covalently connected to an antibody or fragment thereof. In some embodiments, the antibody or fragment thereof comprises a fragment crystallizable (Fc) region. In some embodiments, the engineered polypeptide further comprises a CD59, an endostatin, a VEGF inhibitor, or a combination thereof. In some embodiments, the engineered polypeptide further comprises a CD59, an endostatin, and a VEGF inhibitor. In some embodiments, the engineered polypeptide further comprises a CD59 and an endostatin. In some embodiments, the engineered polypeptide further comprises an endostatin and a VEGF inhibitor. In some embodiments, the engineered polypeptide further comprises an endostatin and a VEGF inhibitor. In some embodiments, the engineered polypeptide further comprises a CD59 and a VEGF inhibitor. In some embodiments, the engineered polypeptide further comprises a CD59, and an endostatin. In some embodiments, the engineered polypeptide comprises a complement 3 inhibitor and an inhibitor of a membrane attack complex (MAC). In some embodiments, the inhibitor of the MAC comprises CD59. In some embodiments, the engineered polypeptide comprises a natriuretic peptide, an endostatin, a VEGF inhibitor, or a combination thereof. In some embodiments, the engineered polypeptide comprises a natriuretic peptide, an endostatin, and a VEGF inhibitor. In some embodiments, the engineered polypeptide comprises a complement 3 inhibitor and a collagen or fragment thereof. In some embodiments, the collagen or fragment thereof comprises an endostatin or fragment thereof. In some embodiments, the engineered polypeptide further comprises a natriuretic peptide, a CD59, a VEGF inhibitor, or a combination thereof. In some embodiments, the engineered polypeptide further comprises a natriuretic peptide. In some embodiments, the engineered polypeptide further comprises a CD59. In some embodiments, the engineered polypeptide further comprises a VEGF inhibitor. In some embodiments, the engineered polypeptide comprises a complement 3 inhibitor and a VEGF inhibitor.

In some embodiments, the engineered polypeptide comprises a CD59, an Fc region, and a natriuretic peptide. In some embodiments, the natriuretic peptide comprises a C-type natriuretic peptide (CNP). In some embodiments, the natriuretic peptide is covalently connected to an antibody or fragment thereof. In some embodiments, the antibody or fragment thereof comprises a fragment crystallizable (Fc) region. In some embodiments, the engineered polypeptide comprises a complement 3 inhibitor, an endostatin, a VEGF inhibitor, or a combination thereof. In some embodiments, the engineered polypeptide comprises a complement 3 inhibitor, an endostatin, and a VEGF inhibitor.

In some embodiments, the engineered polypeptide comprises a CD59 and a collagen or fragment thereof. In some embodiments, the collagen or fragment thereof comprises an endostatin or fragment thereof. In some embodiments, the collagen or fragment thereof comprises an endostatin. In some embodiments, the engineered polypeptide comprises a natriuretic peptide, a complement 3 inhibitor, a VEGF inhibitor, or a combination thereof. In some embodiments, the engineered polypeptide comprises a natriuretic peptide, a complement 3 inhibitor, and a VEGF inhibitor. In some embodiments, the engineered polypeptide comprises a natriuretic peptide and a complement 3 inhibitor.

In some embodiments, the engineered polypeptide comprises a CD59 and a VEGF inhibitor.

In some embodiments, the engineered polypeptide comprises a natriuretic peptide, a complement 3 inhibitor, an endostatin, or a combination thereof. In some embodiments, the engineered polypeptide comprises a natriuretic peptide, a complement 3 inhibitor, and an endostatin.

In some embodiments, the engineered polypeptide comprises sCD59-Fc4-CNP36. In some embodiments, the engineered polypeptide comprises sCD59-C3i-Fc4-CNP36. In some embodiments, the engineered polynucleotide encodes C3i-Fc4-endostatin. In some embodiments, the engineered polypeptide comprises Aflibercept (SEQ ID NO: 71)-linker-C3i. In some embodiments, the engineered polypeptide comprises C3i-Fc4-CNP36-Furin-sCD59. In some embodiments, the engineered polypeptide comprises sCD59-furin 2A-C3i-Fc4-CNP36. In some embodiments, the engineered polypeptide comprises sCD59-furin 2A-endostatin-linker-C3i. In some embodiments, the engineered polypeptide comprises sCD59-furin 2A-Aflibercept-linker-C3i. In some embodiments, the engineered polypeptide comprises C3i-Fc4-CNP36-Furin-mCD59. In some embodiments, the engineered polypeptide comprises C3i-Fc4-endostatin-Furin sCD59. In some embodiments, the engineered polypeptide comprises a sequence as shown in FIG. 1. In some embodiments, the engineered polypeptide comprises a sequence as shown in FIGS. 2A-B. In some embodiments, the engineered polypeptide comprises a sequence as shown in FIG. 3. In some embodiments, the engineered polypeptide comprises a sequence as shown in FIG. 4. In some embodiments, the engineered polypeptide comprises a sequence as shown in FIGS. 5A-C. In some embodiments, the engineered polynucleotide encodes a sequence as shown in FIG. 16. In some embodiments, the engineered polynucleotide encodes a sequence as shown in FIG. 24. In some embodiments, the engineered polynucleotide encodes a sequence as shown in FIG. 26. In some embodiments, the engineered polynucleotide encodes a sequence as shown in FIG. 32.

In some embodiments, the engineered polypeptide can be administered to a subject to treat a disease or condition. In some embodiments, the engineered polypeptide can be formulated into a pharmaceutical composition to be administered to a subject to treat a disease or condition. In some embodiments, the engineered polypeptide can increase activity or signal cascade associated with complement pathway. In some embodiments, the engineered polypeptide can increase activity or signal cascade associated with a natriuretic peptide receptor (NPR). In some embodiments, the engineered polypeptide can increase activity or signal cascade associated with a cyclic GMP (cGMP) signaling pathway. In some embodiments, the engineered polypeptide can increase activity or signal cascade associated with CD59. In some embodiments, the engineered polypeptide can increase activity or signal cascade associated with endostatin. In some embodiments, the engineered polypeptide can decrease activity or signal cascade associated with VEGF.

In some embodiments, the engineered polypeptide can be administered to a subject to treat a disease or condition by increasing activity or signal cascade associated with complement pathway. In some embodiments, the engineered polypeptide can be administered to a subject to treat a disease or condition by increasing activity or signal cascade associated with a natriuretic peptide receptor (NPR). In some embodiments, the engineered polypeptide can be administered to a subject to treat a disease or condition by increasing activity or signal cascade associated with a cGMP signaling pathway. In some embodiments, the engineered polypeptide can be administered to a subject to treat a disease or condition by increasing activity or signal cascade associated with CD59. In some embodiments, the engineered polypeptide can be administered to a subject to treat a disease or condition by increasing activity or signal cascade associated with endostatin. In some embodiments, the engineered polypeptide can be administered to a subject to treat a disease or condition by decreasing activity or signal cascade associated with VEGF.

### Pharmaceutical Composition

Described herein are pharmaceutical compositions comprising an engineered polynucleotide, an AAV vector comprising the engineered polynucleotide, an engineered polypeptide, a cell transduced by an AAV vector comprising an engineered polynucleotide, a viral particle comprising the engineered polynucleotide, or a combination thereof. In some embodiments, the pharmaceutical composition further comprises as pharmaceutically acceptable: carrier, excipient, or diluent. In some embodiments, the pharmaceutical composition comprises two or more active agents as disclosed herein. In some embodiments, the pharmaceutical composition comprising the engineered polynucleotide, the AAV vector comprising the engineered polynucleotide, or the AAV vector comprising the engineered polynucleotide treats a disease or condition described herein. In some embodiments, the disease or condition comprises an ocular disease. In some embodiments, the disease or condition comprises ocular ischemic syndrome, proliferative retinopathies, neovascular glaucoma (NG), uveitis, neovascular uveitis, achromatopsia, age-related macular degeneration (nAMD), diabetic macular edema (DME), diabetic macular retinopathy (DMR), retinal vein occlusion (RVO), glaucoma, traumatic glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

For in vivo delivery, the engineered polynucleotide, the AAV vector comprising the engineered polynucleotide, the engineered polypeptide, the cell transduced by an AAV vector comprising the engineered polynucleotide, or a combination thereof can be formulated into pharmaceutical compositions and can generally be administered intravitreally or parenterally (e.g., administered via an intramuscular, subcutaneous, intratumoral, transdermal, intrathecal, etc., route of administration). In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof to a subject in need thereof.

In some aspects, a pharmaceutical composition can be used to treat a subject such as a human or mammal, in need thereof. In some cases, a subject can be diagnosed with a disease, e.g., ocular disease. In some aspects, subject pharmaceutical compositions are co-administered with secondary therapies. A secondary therapy can comprise any therapy for ocular use. In some cases, a secondary therapy comprises nutritional therapy, vitamins, laser treatment, such as laser photocoagulation, photodynamic therapy, Visudyne, anti-VEGF therapy, eye-wear, eye drops, numbing agents, Orthoptic vision therapy, Behavioral/perceptual vision therapy, and the like. In some aspects, any of the previously described biologics can be considered a secondary therapy.

In some embodiments, an effective amount of the pharmaceutical composition results in a decrease in the rate of loss of retinal function, anatomical integrity, or retinal health, e.g. a 2-fold, 3-fold, 4-fold, or 5-fold or more decrease in the rate of loss and hence progression of disease, for example, a 10-fold decrease or more in the rate of loss and hence progression of disease.

In some embodiments, an effective amount of the pharmaceutical composition decreases neovascularization signaling in a cell by at least 10%, at least 20%, at least 30%, at least 40%, at least 50%, at least 60%, at least 70%, at least 80%, at least 90%, at least 100%, at least 200%, at least 500%, or more compared to neovascularization signaling in a cell that is not treated with the pharmaceutical composition. In some embodiments, an effective amount of the pharmaceutical composition decreases neovascularization in a subject in need thereof at least 10%, at least 20%, at least 30%, at least 40%, at least 50%, at least 60%, at least 70%, at least 80%, at least 90%, at least 100%, at least 200%, at least 500%, or more compared to neovascularization in the subject if the subject is not treated with the pharmaceutical composition. In some embodiments, an effective amount of the pharmaceutical composition decreases blood vessel leakage in a subject in need thereof at least 10%, at least 20%, at least 30%, at least 40%, at least 50%, at least 60%, at least 70%, at least 80%, at least 90%, at least 100%, at least 200%, at least 500%, or more compared to blood vessel leakage in the subject if the subject is not treated with the pharmaceutical composition. In some embodiments, an effective amount of the pharmaceutical composition decreases inflammation in a subject in need thereof at least 10%, at least 20%, at least 30%, at least 40%, at least 50%, at least 60%, at least 70%, at least 80%, at least 90%, at least 100%, at least 200%, at least 500%, or more compared to inflammation in the subject if the subject is not treated with the pharmaceutical composition.

In some embodiments, the effective amount of the subject rAAV virion results in a gain in visual function, retinal function, an improvement in retinal anatomy or health, and/or an improvement in ocular motility and/or improvement in neurological function, e.g. a 2-fold, 3-fold, 4-fold or 5-fold improvement or more in retinal function, retinal anatomy or health, and/or improvement in ocular motility, e.g. a 10-fold improvement or more in retinal function, retinal anatomy or health, and/or improvement in ocular motility. As will be readily appreciated by the ordinarily skilled artisan, the dose required to achieve the desired treatment effect will typically be in the range of 1×10⁸ to about 1×10¹⁵ recombinant virions, typically referred to by the ordinarily skilled artisan as 1×10⁸ to about 1×10¹⁵ “vector genomes”.

In some aspects, compositions provided herein, such as pharmaceutical compositions are administered to a subject in need thereof. In some cases, an administration comprises delivering a dosage of an AAV of about vector 0.5×10⁹ vg, 1.0×10⁹ vg, 1.0×10¹⁰, 1.0×10¹¹ vg, 3.0×10¹¹ vg, 6×10¹¹ vg, 8.0×10¹¹ vg, 1.0×10¹² vg, 1.0×10¹³ vg, 1.0×10¹⁴ vg, 1.0×10¹⁵ vg, 1.5×10¹⁵ vg. For example, for in vivo injection, e.g., injection directly into the eye, a therapeutically effective dose can be on the order of from about 10⁶ to about 10¹⁵ of subject AAV virions, e.g., from about 10⁸ to 10¹² engineered AAV virions. For in vitro transduction, an effective amount of engineered AAV virions to be delivered to cells will be on the order of from about 10⁸ to about 10¹³ of the engineered AAV virions. Other effective dosages can be readily established by one of ordinary skill in the art through routine trials establishing dose response curves.

Administrations can be repeated for any amount of time. In some aspects, administering is performed: twice daily, every other day, twice a week, bimonthly, trimonthly, once a month, every other month, semiannually, annually, or biannually.

Dosage treatment may be a single dose schedule or a multiple dose schedule. Moreover, the subject may be administered as many doses as appropriate. One of skill in the art can readily determine an appropriate number of doses. In some aspects, a pharmaceutical composition is administered via intravitreal injection, subretinal injection, microinjection, or supraocular injection.

In practicing the methods of treatment or use provided herein, therapeutically effective amounts of the pharmaceutical composition described herein are administered to a mammal having a disease, disorder, or condition to be treated, e.g., cancer. In some embodiments, the mammal is a human. A therapeutically effective amount may vary widely depending on the severity of the disease, the age and relative health of the subject, the potency of the therapeutic agent used and other factors. The therapeutic agents, and in some cases, compositions described herein, may be used singly or in combination with one or more therapeutic agents as components of mixtures.

The pharmaceutical composition described herein may be administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The composition described herein may include, but not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical composition may be manufactured in a conventional manner, such as, by way of example only, by means of conventional mixing, dissolving, granulating, levigating, emulsifying, encapsulating, entrapping or compression processes.

In certain embodiments, the pharmaceutical composition provided herein includes one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, the pharmaceutical composition described herein is formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for rehydration into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms may be ensured by various antibacterial and antifungal agents, such as parabens, chlorobutanol, phenol, sorbic acid, and the like. In some cases, it is desirable to include isotonic agents, such as sugars, sodium chloride, and the like. Prolonged absorption of the injectable pharmaceutical form may be brought about by the use of agents delaying absorption, such as aluminum monostearate and gelatin.

In another aspect, dosage forms include microencapsulated formulations. In some embodiments, one or more other compatible materials are present in the microencapsulation material. Non-limiting example of materials includes pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Liquid formulation dosage forms for oral administration are optionally aqueous suspensions selected from the group including, but not limited to, pharmaceutically acceptable aqueous oral dispersions, emulsions, solutions, elixirs, gels, and syrups. In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions\ further includes a crystal-forming inhibitor.

In some embodiments, the pharmaceutical composition described herein is self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients.

Furthermore, the pharmaceutical composition optionally includes one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, the pharmaceutical composition optionally includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

### Kit

Disclosed herein, in some embodiments, are kits for using comprising an engineered polynucleotide, an AAV comprising the engineered polynucleotide, an engineered polypeptide, a cell transduced by an AAV vector comprising an engineered polynucleotide, a viral particle comprising the engineered polynucleotide, a pharmaceutical composition, or a combination thereof described herein. In some embodiments, the kit disclosed herein may be used to treat a disease or condition in a subject. In some embodiments, the kit comprises an assemblage of materials or components apart from comprising the engineered polynucleotide, the AAV comprising the engineered polynucleotide, the engineered polypeptide, the cell transduced by an AAV vector comprising an engineered polynucleotide, or the pharmaceutical composition.

In some embodiments, the kit described herein comprises components for selecting for a homogenous population of AAV containing the engineered polynucleotide described herein. In some embodiments, the kit comprises the components for assaying the number of units of a biomolecule (e.g., the AAV) synthesized, and/or released or expressed on the surface by a host cell. In some embodiments, the kit comprises components for performing assays such as enzyme-linked immunosorbent assay (ELISA). The exact nature of the components configured in the kit depends on its intended purpose. For example, some embodiments are configured for the purpose of treating a disease or condition disclosed herein (e.g., cancer) in a subject. In some embodiments, the kit is configured particularly for the purpose of treating mammalian subjects. In some embodiments, the kit is configured particularly for the purpose of treating human subjects.

Instructions for use may be included in the kit. In some embodiments, the kit comprises instructions for administering the engineered polynucleotide, the AAV vector comprising the engineered polynucleotide, the engineered polypeptide, the AAV comprising the engineered polynucleotide, the cell transduced with the AAV vector, the pharmaceutical composition, or a combination thereof to a subject in need thereof. In some embodiments, the kit comprises instructions for further engineering a cell to express a biomolecule (e.g., the engineered polynucleotide, the AAV vector comprising the engineered polynucleotide, the engineered polypeptide, the AAV comprising the engineered polynucleotide, or the cell transduced with the AAV vector). In some embodiments, the kit comprises instructions for thawing or otherwise restoring biological activity of the engineered polynucleotide, the AAV vector comprising the engineered polynucleotide, AAV comprising the engineered polynucleotide, which may have been cryopreserved or lyophilized during storage or transportation. In some embodiments, the kit comprises instructions for measuring the viability of the restored the engineered polynucleotide, the AAV vector comprising the engineered polynucleotide, AAV comprising the engineered polynucleotide to ensure efficacy for its intended purpose (e.g., therapeutic efficacy if used for treating a subject).

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are typically contained in suitable packaging material(s).

### Method of Delivery

The engineered polynucleotide can be readily introduced into a host cell, e.g., a mammalian, bacterial, yeast, or insect cell by any method in the art. For example, the engineered polynucleotide can be transferred into a host cell by physical, chemical, or biological means. In some embodiments, the engineered polynucleotide can be delivered to a host cell by encapsulating the engineered polynucleotide in a viral particle such as an AAV particle. In some embodiments, the engineered polynucleotide can be delivered into the cell via physical methods such as calcium phosphate precipitation, lipofection, particle bombardment, microinjection, gene gun, electroporation, and the like.

Physical methods for introducing the engineered polynucleotide encoding into the cell can include calcium phosphate precipitation, lipofection, particle bombardment, microinjection, gene gun, electroporation, and the like. One method for the introduction of the engineered polynucleotide a host cell is calcium phosphate transfection.

Chemical means for introducing the engineered polynucleotide encoding the non-naturally into the cell can include colloidal dispersion systems, such as macromolecule complexes, nanocapsules, microspheres, beads, and lipid-based systems including oil-in-water emulsions, micelles, mixed micelles, spherical nucleic acid (SNA), liposomes, or lipid nanoparticles. An example colloidal system for use as a delivery vehicle in vitro and in vivo is a liposome (e.g., an artificial membrane vesicle). Other methods of state-of-the-art targeted delivery of nucleic acids are available, such as delivery of engineered polynucleotide or vector encoding the engineered polynucleotide with targeted nanoparticles.

In the case where a non-viral delivery system is utilized, an example delivery vehicle is a liposome. The use of lipid formulations is contemplated for the introduction of the engineered polynucleotide or vector encoding the engineered polynucleotide into a cell (in vitro, ex vivo, or in vivo). In another aspect, the vector can be associated with a lipid. The vector associated with a lipid can be encapsulated in the aqueous interior of a liposome, interspersed within the lipid bilayer of a liposome, attached to a liposome via a linking molecule that is associated with both the liposome and the engineered polynucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, in some embodiments, they are present in a bilayer structure, as micelles, or with a “collapsed” structure. Alternately, they are simply be interspersed in a solution, possibly forming aggregates that are not uniform in size or shape. Lipids are fatty substances which are, in some embodiments, naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use are obtained from commercial sources. Stock solutions of lipids in chloroform or chloroform/methanol are often stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes are often characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids, in some embodiments, assume a micellar structure or merely exist as nonuniform aggregates of lipid molecules. Also contemplated are lipofectamine-nucleic acid complexes.

In some cases, non-viral delivery method comprises lipofection, nucleofection, microinjection, biolistics, virosomes, liposomes, immunoliposomes, exosomes, polycation or lipid: cargo conjugates (or aggregates), naked polypeptide (e.g., recombinant polypeptides), naked DNA, artificial virions, and agent-enhanced uptake of polypeptide or DNA. In some embodiments, the delivery method comprises conjugating or encapsulating the compositions or the engineered polynucleotides described herein with at least one polymer such as natural polymer or synthetic materials. The polymer can be biocompatible or biodegradable. Non-limiting examples of suitable biocompatible, biodegradable synthetic polymers can include aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amine groups, and poly(anhydrides). Such synthetic polymers can be homopolymers or copolymers (e.g., random, block, segmented, graft) of a plurality of different monomers, e.g., two or more of lactic acid, lactide, glycolic acid, glycolide, epsilon-caprolactone, trimethylene carbonate, p-dioxanone, etc. In an example, the scaffold can be comprised of a polymer comprising glycolic acid and lactic acid, such as those with a ratio of glycolic acid to lactic acid of 90/10 or 5/95. Non-limiting examples of naturally occurring biocompatible, biodegradable polymers can include glycoproteins, proteoglycans, polysaccharides, glycosamineoglycan (GAG) and fragment(s) derived from these components, elastin, laminins, decrorin, fibrinogen/fibrin, fibronectins, osteopontin, tenascins, hyaluronic acid, collagen, chondroitin sulfate, heparin, heparan sulfate, ORC, carboxymethyl cellulose, and chitin.

In some cases, the engineered polynucleotide described herein can be packaged and delivered to the cell via extracellular vesicles. The extracellular vesicles can be any membrane-bound particles. In some embodiments, the extracellular vesicles can be any membrane-bound particles secreted by at least one cell. In some instances, the extracellular vesicles can be any membrane-bound particles synthesized in vitro. In some instances, the extracellular vesicles can be any membrane-bound particles synthesized without a cell. In some cases, the extracellular vesicles can be exosomes, microvesicles, retrovirus-like particles, apoptotic bodies, apoptosomes, oncosomes, exophers, enveloped viruses, exomeres, or other very large extracellular vesicles.

In some embodiments, the engineered polynucleotide can be delivered into the cell via biological methods such as the use of DNA and RNA vectors. Viral vectors, and especially retroviral vectors, have become the most widely used method for inserting genes into mammalian, e.g., human cells. Other viral vectors, in some embodiments, are derived from lentivirus, poxviruses, herpes simplex virus I, adenoviruses and adeno-associated viruses, and the like. Exemplary viral vectors include retroviral vectors, adenoviral vectors, adeno-associated viral vectors (AAV vectors), pox vectors, parvoviral vectors, baculovirus vectors, measles viral vectors, or herpes simplex virus vectors (HSVs). In some instances, the retroviral vectors include gamma-retroviral vectors such as vectors derived from the Moloney Murine Keukemia Virus (MoMLV, MMLV, MuLV, or MLV) or the Murine Steam cell Virus (MSCV) genome. In some instances, the retroviral vectors also include lentiviral vectors such as those derived from the human immunodeficiency virus (HIV) genome. In some instances, AAV comprises a serotype, including AAV1, AAV2, AAV4, AAV5, AAV6, AAV7, AAV8, AAV9, AAV10, AAV11, AAV12, or a combination thereof. Based on these initial serotypes, AAV capsid of each serotype can be engineered to make them better suited for biological functions, tissue or cell selection. In some embodiments, an AAV is AAV2 and variants AAV2.N53 and AAV2.N54 which are used in the examples of the present disclosure. Chimeric AAVs are also contemplated that may contain at least 2 AAV serotypes. In some cases, at least 3, at least 4, at least 5, at least 6, at least 7, or up to 8 different serotypes are combined in a chimeric AAV. In some cases, only a portion of the AAV is chimeric. For example, suitable portions can include the capsid, VP1, VP2, or VP3 domains and/or Rep. In some cases, at least one of VP1, VP2, and VP3 has at least one amino acid substitution compared to an otherwise comparable wild-type AAV capsid protein. In some cases, a mutation can occur in VP1 and VP2, in VP1 and VP3, in VP2 and VP3, or in VP1, VP2, and VP3. In some embodiments, at least one of VP1, VP2, and VP3 has from one to about 25 amino acid substitutions compared to wild-type AAV VP1, VP2, and VP3, e.g., from about one to about 5, from about 5 to about 10, from about 10 to about 15, from about 15 to about 20, or from about 20 to about 25 amino acid substitutions compared to wild-type AAV VP1, VP2, and VP3. In some cases, a VP can be removed. For example, in some embodiments a mutant AAV does not comprise at least one of VP1, VP2, or VP3.

### Method of Modifying Cell

In an aspect, provided herein are also methods of modifying cells to thereby generate engineered cells. Cells can refer to primary cells, recombinant cells, or cell lines. In some cases, a cell is a packaging cell. A packaging cell can be any one of: HEK 293 cells, HeLa cells, and Vero cells to name a few. An engineered cell can be a primary cell. In some cases, an engineered cell can be an ocular cell. Suitable ocular cells include but are not limited to a: photoreceptor, ganglion cell, RPE cell, amacrine cell, horizontal cell, muller cell, and the like.

In some cases, a cell is a packaging cell utilized to generate viral particles. To generate AAV virions or viral particles, an AAV vector is introduced into a suitable host cell using known techniques, such as by transfection. In some cases, transfection techniques are used, e.g., CaPO4 transfection or electroporation, and/or infection by hybrid adenovirus/AAV vectors into cell lines such as the human embryonic kidney cell line HEK 293 (a human kidney cell line containing functional adenovirus E1 genes which provides trans-acting E1 proteins). Suitable transfection methods include calcium phosphate co-precipitation, direct micro-injection, electroporation, liposome mediated gene transfer, and nucleic acid delivery using high-velocity microprojectiles, which are known in the art.

To engineer a cell, a plurality of cells may be contacted with an isolated engineered polynucleotide. Contacting can comprise any length of time and may include from about 5 min to about 5 days. Contacting can last from about 5, about 10, about 15, about 20, about 25, about 30, about 35, about 40, about 45, about 50, about 55, or about 60 minutes. In some cases, the contacting can last from 1 hour, 3 hours, 5 hours, 10 hours, 15 hours, 20 hours, 1 day, 2 days, 3 days, 4 days or up to about 5 days.

In some cases, supernatant of the packaging cell line is treated by PEG precipitation for concentrating the virus. In other cases, a centrifugation step can be used to concentrate a virus. For example, a column can be used to concentration a virus during a centrifugation. In some embodiments, a precipitation occurs at no more than about 4° C. (for example about 3° C., about 2° C., about 1° C., or about 1° C.) for at least about 2 hours, at least about 3 hours, at least about 4 hours, at least about 6 hours, at least about 9 hours, at least about 12 hours, or at least about 24 hours. In some embodiments, the recombinant AAV is isolated from the PEG-precipitated supernatant by low-speed centrifugation followed by CsCl gradient. The low-speed centrifugation can be to can be about 4000 RPM, about 4500 RPM, about 5000 RPM, or about 6000 RPM for about 20 minutes, about 30 minutes, about 40 minutes, about 50 minutes or about 60 minutes. In some cases, recombinant AAV is isolated from the PEG-precipitated supernatant by centrifugation at about 5000 RPM for about 30 minutes followed by CsCl gradient. In some cases, CsCl purification can be replaced with IDX gradient ultracentrifugation. Supernatant can be collected at about 12 hours, about 24 hours, about 36 hours, about 48 hours, about 72 hours, about 96 hours, about 120 hours, or a time between any of these two time points after a transfection. Supernatant can also be purified, concentrated, or a combination thereof. For example, a concentration or viral titer can be determined by qPCR or silver stain.

In an aspect, provided is also a plurality of AAV particles (containing the engineered polynucleotide described herein) isolated from an engineered cell. A viral titer can be from about 10² vp/mL, about 10³ vp/mL, about 10⁴ vp/mL, about 10⁵ vp/mL, about 10⁶ vp/mL, about 10⁷ vp/mL, about 10⁸ vp/mL, or up to about 10⁹ vp/mL. A viral titer can be from about 10² GC/mL, about 10³ GC/mL, about 10⁴ GC/mL, about 10⁵ GC/mL, about 10⁶ GC/mL, about 10⁷ GC/mL, about 10⁸ GC/mL, or up to about 10⁹ GC/mL. In some cases, a viral titer can be from about 10² TU/mL, about 10³ TU/mL, about 10⁴ TU/mL, about 10⁵ TU/mL, about 10⁶ TU/mL, about 10⁷ TU/mL, about 10⁸ TU/mL, or up to about 10⁹ TU/mL. An optimal viral titer can vary depending on cell type to be transduced. A range of virus can be from about 1000 MOI to about 2000 MOI, from about 1500 MOI to about 2500 MOI, from about 2000 MOI to about 3000 MOI, from about 3000 MOI to about 4000 MOI, from about 4000 MOI to about 5000 MOI, from about 5000 MOI to about 6000 MOI, from about 6000 MOI to about 7000 MOI, from about 7000 MOI to about 8000 MOI, from about 8000 MOI to about 9000 MOI, from about 9000 MOI to about 10,000 MOI. For example, to infect 1 million cells using a MOI of 10,000, one will need 10,000×1,000,000=10¹⁰ GC.

In some cases, a plurality of AAV particles can be formulated into unit dose form. Various formulations are contemplated for adult or pediatric delivery and include but are not limited to: 0.5×10⁹ vg, 1.0×10⁹ vg, 1.0×10¹⁰, 1.0×10¹¹ vg, 3.0×10¹¹ vg, 6×10¹¹ vg, 8.0×10¹¹ vg, 1.0×10¹² vg, 1.0×10¹³ vg, 1.0×10¹⁴ vg, 1.0×10¹⁵ vg, or up to 1.5×10¹⁵ vg. Compositions of viral particles can be cryopreserved or otherwise stored in suitable containers.

Provided compositions and methods herein can be sufficient to enhance delivery and/or expression of subject biologic by at least about 3%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or up to 100% more than an otherwise comparable unmodified nucleic acid. In some cases, the otherwise comparable unmodified nucleic acid is one that encodes VEGF-Trap. In some cases, modifications can be sufficient to enhance delivery and/or expression of subject biologics by at least about 1-fold, about 6-fold, about 11-fold, about 16-fold, about 21-fold, about 26-fold, about 31-fold, about 36-fold, about 41-fold, about 46-fold, about 51-fold, about 56-fold, about 61-fold, about 66-fold, about 71-fold, about 76-fold, about 81-fold, about 86-fold, about 91-fold, about 96-fold, about 101-fold, about 106-fold, about 111-fold, about 116-fold, about 121-fold, about 126-fold, about 131-fold, about 136-fold, about 141-fold, about 146-fold, about 151-fold, about 156-fold, about 161-fold, about 166-fold, about 171-fold, about 176-fold, about 181-fold, about 186-fold, about 191-fold, about 196-fold, about 201-fold, about 206-fold, about 211-fold, about 216-fold, about 221-fold, about 226-fold, about 231-fold, about 236-fold, about 241-fold, about 246-fold, about 251-fold, about 256-fold, about 261-fold, about 266-fold, about 271-fold, about 276-fold, about 281-fold, about 286-fold, about 291-fold, about 296-fold, about 301-fold, about 306-fold, about 311-fold, about 316-fold, about 321-fold, about 326-fold, about 331-fold, about 336-fold, about 341-fold, about 346-fold, or about 350-fold more than an otherwise comparable unmodified nucleic acid. In an embodiment, increased expression comprises at least a 5-fold, at least a 10-fold, at least a 20-fold, at least a 50-fold, at least a 100-fold, at least a 200-fold, or at least a 500-fold increase as determined by in in vitro assay. Suitable in vitro assays include ELISA, western blot, Luminex, microscopy, imaging, and/or flow cytometry.

A subject AAV virion can exhibit at least 1-fold, at least 6-fold, at least 10-fold, at least 15-fold, at least 20-fold, at least 25-fold, at least 50-fold, or more than 50-fold, increased infectivity of a retinal cell, compared to the infectivity of the retinal cell (photoreceptor, ganglion cell, RPE cell, amacrine cell, horizontal cell, muller cell, and the like) by an AAV virion comprising an otherwise comparable WT AAV capsid protein.

### Method of Treatment

Provided herein are methods of treating a disease or condition described here. In some aspects, the method confers protection against the disease or condition. A method of treatment can comprise introducing to a subject in need an engineered polynucleotide, an engineered polypeptide, an AAV vector comprising the engineered polynucleotide, an AAV comprising the engineered polynucleotide, a cell transduced with an AAV vector, a viral particle comprising the engineered polynucleotide, a pharmaceutical composition, or a combination thereof. Also provided is a method of treating disease or condition that comprises administering a pharmaceutical composition to a subject in need thereof. A pharmaceutical composition can comprise a sequence that encodes a biologic that comprises the engineered polynucleotide, the AAV vector comprising the engineered polynucleotide, the AAV vector comprising the engineered polynucleotide, a viral particle comprising the engineered polynucleotide, or a combination thereof. In some embodiments, administration is by any suitable mode of administration, including systemic administration (e.g., intravenous, intravitreal, subretinal, or etc.). In some embodiments, the subject is human.

In some embodiments, the method comprises treating a disease or condition in a subject in need thereof by administering to the subject a therapeutically effective amount of an engineered polynucleotide, an engineered polypeptide, a cell transduced with an engineered polynucleotide, or pharmaceutical composition described herein. In some embodiments, the method treats a disease or condition, where once of the administering of an engineered polynucleotide, an engineered polypeptide, a cell transduced with an engineered polynucleotide, or pharmaceutical composition described herein is curative of the disease or condition. In some embodiments, the method treats a disease or condition, where the administering of an engineered polynucleotide, an engineered polypeptide, a cell transduced with an engineered polynucleotide, or pharmaceutical composition described herein does not comprise daily administration. In some embodiments, the disease or condition comprises an ocular disease. Non-limiting example of the ocular disease can include ocular ischemic syndrome, proliferative retinopathies, neovascular glaucoma (NG), uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), glaucoma, traumatic glaucoma, Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof. In some embodiments, the disease or condition is neovascular glaucoma (NG). In some embodiments, the disease or condition is glaucoma. In some embodiments, the disease or condition is traumatic glaucoma.

In another aspect, provided herein is a pharmaceutical composition comprising an engineered polynucleotide disclosed herein, an engineered polypeptide disclosed herein, a vector disclosed herein, a viral particle disclosed herein, a cell disclosed herein, or a composition disclosed herein. In some embodiments, the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof. In some embodiments, the pharmaceutical composition is formulated for administering intrathecally. In some embodiments, the pharmaceutical composition is formulated for administering retinally. In some embodiments, the pharmaceutical composition is formulated for administering intraocularly. In some embodiments, the pharmaceutical composition is formulated for administering intravitreally, subretinally, or suprachoroidally.

In another aspect, provided herein is a method comprising contacting a cell obtained from a subject with an engineered polynucleotide disclosed herein, an engineered polypeptide disclosed herein, a vector disclosed herein, a viral particle disclosed herein, a cell disclosed herein, a composition disclosed herein, or a pharmaceutical composition disclosed herein.

In another aspect, provided herein is a method of treating a disease or condition in a subject, comprising: administering to the subject an engineered polynucleotide disclosed herein, an engineered polypeptide disclosed herein, a vector disclosed herein, a viral particle disclosed herein, a cell disclosed herein, a composition disclosed herein, or a pharmaceutical composition disclosed herein. In some embodiments, the administering is curative of the disease or condition. In some embodiments, the administering does not comprise daily administration. In some embodiments, the administering comprises a weekly administration, a bi-weekly administration, a monthly administration, a bi-month administration, a semiannual administration, an annual administration, or a combination thereof. In some embodiments, the disease or condition comprises an ocular disease. In some embodiments, the ocular disease comprises ocular ischemic syndrome, proliferative retinopathies, neovascular glaucoma (NG), glaucoma, traumatic glaucoma, uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof. In some embodiments, the ocular disease comprises GA or dAMD.

In another aspect, provided herein is a method of treating a disease or condition in a subject, the method comprising administering to the subject an engineered polynucleotide comprising one or more expression cassettes, the one or more expression cassettes encoding a first angiogenesis inhibitor and a second angiogenesis inhibitor. In some embodiments, the first angiogenesis inhibitor and the second angiogenesis inhibitor is each encoded by an expression cassette of the one or more expression cassettes. In some embodiments, the first angiogenesis inhibitor and the second angiogenesis inhibitor are operatively coupled. In some embodiments, the first angiogenesis inhibitor and the second angiogenesis inhibitor are covalently connected by a linker. In some embodiments, the first angiogenesis inhibitor comprises a complement inhibitor such as a complement 3 inhibitor. In some embodiments, the complement 3 inhibitor comprises an amino acid sequence that is at least 80% identical to any one of SEQ ID NOs: 1-15. In some embodiments, the complement 3 inhibitor is encoded by a nucleic acid sequence that is at least 80% identical to any one of SEQ ID NOs: 20-33. In some embodiments, the first angiogenesis inhibitor comprises an inhibitor of a membrane attack complex (MAC) such as CD59. In some embodiments, the CD59 comprises an amino acid sequence that is at least 80% identical to any one of SEQ ID NOs: 41-45. In some embodiments, the second angiogenesis inhibitor comprises a natriuretic peptide. In some embodiments, the natriuretic peptide comprises an amino acid sequence that is at least 80% identical to any one of SEQ ID NOs: 61-72. In some embodiments, the second angiogenesis inhibitor comprises a collagen or fragment thereof. In some embodiments, the second angiogenesis inhibitor comprises an endostatin or fragment thereof. In some embodiments, the second angiogenesis inhibitor comprises an amino acid sequence that is at least 80% identical to SEQ ID NO: 51. In some embodiments, the second angiogenesis inhibitor comprises a VEGF inhibitor. In some embodiments, the VEGF inhibitor comprises an amino acid sequence that is at least 80% identical to any one of SEQ ID NOs: 81-92.

In some embodiments, the engineered polynucleotide comprises a viral vector. In some embodiments, the viral vector comprises an AAV vector. In some embodiments, the AAV vector comprises an AAV serotype comprising AAV1, AAV2, AAV3, AAV4, AAV5, AAV6, AAV7, AAV8, AAV9, AAV10, AAV11, AAV12, or any combination thereof. In some embodiments, the AAV vector is an AAV2 vector. In some embodiments, the AAV vector encodes an engineered AAV capsid, where the engineered AAV capsid comprises an amino acid sequence of any one of SEQ ID NOs: 161-182 and SEQ ID NOs: 191-210. In some embodiments, the engineered AAV capsid comprises the amino acid sequence of SEQ ID NO: 169.

In some embodiments, the method comprises once of the administering being curative of the disease or condition. In some embodiments, the administering does not comprise daily administration. In some embodiments, the administering comprises a weekly administration, a bi-weekly administration, a monthly administration, a bi-month administration, a semiannual administration, an annual administration, or a combination thereof. In some embodiments, the disease or condition comprises an ocular disease such as ocular ischemic syndrome, proliferative retinopathies, neovascular glaucoma (NG), glaucoma, traumatic glaucoma, uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof. In some embodiments, the ocular disease comprises GA or dAMD. In some embodiments, the first angiogenesis inhibitor and the second angiogenesis inhibitor, upon administered to a subject, inhibits neovascularization in the subject.

In some embodiments, the first angiogenesis inhibitor or the second angiogenesis inhibitor, upon administered to the subject, exhibits decreased inhibition of neovascularization in the subject compared to inhibition of neovascularization caused by a VEGF inhibitor. In such case, the decreased inhibition of neovascularization induced by the first angiogenesis inhibitor or the second angiogenesis can be more therapeutically effective in treating the disease or condition. For example, the decreased inhibition of neovascularization allows presence of blood vessels for transporting and delivering the first angiogenesis inhibitor or the second angiogenesis to a site associated with the disease or condition.

In some embodiments, administering a therapeutically effective amount of an engineered polynucleotide, an engineered polypeptide, a cell transduced with an engineered polynucleotide, or pharmaceutical composition described herein to a subject protects the subjection from the disease or condition. For example, administering a therapeutically effective amount of an engineered polynucleotide, an engineered polypeptide, a cell transduced with an engineered polynucleotide, or pharmaceutical composition can protect the subject from developing disease or condition stemmed from injury. In some embodiments, administering a therapeutically effective amount of an engineered polynucleotide, an engineered polypeptide, a cell transduced with an engineered polynucleotide, or pharmaceutical composition protects or promotes survival of cells in a subject. In some embodiments, administering a therapeutically effective amount of an engineered polynucleotide, an engineered polypeptide, a cell transduced with an engineered polynucleotide, or pharmaceutical composition protects or promotes survival of ocular cells in a subject. In some embodiments, administering a therapeutically effective amount of an engineered polynucleotide, an engineered polypeptide, a cell transduced with an engineered polynucleotide or, pharmaceutical composition protects or promotes survival of retinal ganglion cells in a subject.

In some embodiments, administering a therapeutically effective amount of an engineered polynucleotide, an engineered polypeptide, a cell transduced with an engineered polynucleotide or, pharmaceutical composition decreases intraocular pressure in a subject.

In some embodiments, the engineered polynucleotide, the engineered polypeptide, the AAV vector comprising the engineered polynucleotide, the AAV comprising the engineered polynucleotide, the cell transduced with the AAV vector, or the pharmaceutical composition is administered at least once during a period of time (e.g., every 2 days, twice a week, once a week, every week, three times per month, two times per month, one time per month, every 2 months, every 3 months, every 4 months, every 5 months, every 6 months, every 7 months, every 8 months, every 9 months, every 10 months, every 11 months, once a year). In some embodiments, the composition is administered two or more times (e.g., 2, 3, 4, 5, 6, 7, 8, 9, 10, 11, 12, 13, 14, 15, 16, 17, 18, 19, 20, 30, 40, 50, 60, 70, 80, 90, 100 times) during a period of time. In some embodiments, the administration described herein comprises a single administration. In some embodiments, the administration described herein does not include daily administration.

In some embodiments, the method comprises administering the engineered polynucleotide, the engineered polypeptide, the AAV vector comprising the engineered polynucleotide, the AAV comprising the engineered polynucleotide, the cell transduced with the AAV vector, or the pharmaceutical composition in a therapeutically-effective amount by various forms and routes including, for example, oral, or topical administration. In some embodiments, a composition may be administered by intravitreal, subretinal, suprachoroidal, parenteral, intravenous, subcutaneous, intramuscular, intradermal, intraperitoneal, intracerebral, subarachnoid, intraocular, intrasternal, ophthalmic, endothelial, local, intranasal, intrapulmonary, rectal, intraarterial, intrathecal, inhalation, intralesional, intradermal, epidural, intracapsular, subcapsular, intracardiac, transtracheal, subcuticular, subarachnoid, or intraspinal administration, e.g., injection or infusion. In some embodiments, a composition may be administered by absorption through epithelial or mucocutaneous linings (e.g., oral mucosa, rectal and intestinal mucosa administration). In some embodiments, the composition is delivered via multiple administration routes.

In some embodiments, the method comprises administering the engineered polynucleotide, the engineered polypeptide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof by intravenous infusion. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof is administered by slow continuous infusion over a long period, such as more than 24 hours. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof is administered as an intravenous injection or a short infusion. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof is administered via vitreous route. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof may be administered in a local manner, for example, via injection of the agent directly into an organ, optionally in a depot or sustained release formulation or implant.

In some embodiments, the engineered polynucleotide, the engineered polypeptide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof may be administered in conjunction with other therapies, for example, an antiviral therapy, a chemotherapy, an antibiotic, a cell therapy, a cytokine therapy, or an anti-inflammatory agent. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof may be administered before, during, or after the occurrence of a disease or condition, and the timing of administering the composition containing a therapeutic agent may vary. In some cases, the composition may be used as a prophylactic and may be administered continuously to subjects (e.g., the subject for immunization or the subject for treatment) with a susceptibility to a coronavirus or a propensity to a condition or disease associated with a coronavirus. Prophylactic administration may lessen a likelihood of the occurrence of the infection, disease or condition, or may reduce the severity of the infection, disease or condition.

The engineered polynucleotide, the engineered polypeptide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof may be administered to a subject before the onset of the symptoms. In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof may be administered to a subject (e.g., the subject for immunization or the subject for treatment) after (e.g., as soon as possible after) a test result, for example, a test result that provides a diagnosis, a test that shows the presence of a coronavirus in a subject (e.g., the subject for immunization or the subject for treatment), or a test showing progress of a condition, e.g., a decreased blood oxygen levels. A therapeutic agent may be administered after (e.g., as soon as is practicable after) the onset of a disease or condition is detected or suspected. A therapeutic agent may be administered after (e.g., as soon as is practicable after) a potential exposure to a coronavirus, for example, after a subject (e.g., the subject for immunization or the subject for treatment) has contact with an infected subject, or learns they had contact with an infected subject that may be contagious.

Actual dosage levels of an agent of the disclosure (e.g., the engineered polynucleotide or a pharmaceutical composition) may be varied so as to obtain an amount of the agent to achieve the desired therapeutic response for a particular subject, composition, and mode of administration, without being toxic to the subject (e.g., the subject for immunization or the subject for treatment). The selected dosage level may depend upon a variety of pharmacokinetic factors including the activity of the particular compositions of the present disclosure employed, the route of administration, the time of administration, the rate of excretion, the duration of the treatment, other drugs, compounds and/or materials used in combination with the particular compositions employed, the age, sex, weight, condition, general health and prior medical history of the patient being treated, and like factors well known in the medical arts.

Dosage regimens may be adjusted to provide the optimum desired response (e.g., a therapeutic and/or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects (e.g., the subjects for immunization or the subjects for treatment); each unit contains a predetermined quantity of active agent calculated to produce the desired therapeutic effect in association with the required pharmaceutical carrier. The specification for the dosage unit forms of the disclosure may be determined by and directly dependent on (a) the unique characteristics of the active agent and the particular therapeutic effect to be achieved, and (b) the limitations inherent in the art of compounding such an active agent for the treatment of sensitivity in individuals. A dose may be determined by reference to a plasma concentration or a local concentration of the circular polyribonucleotide or antibody or antigen-binding fragment thereof. A dose may be determined by reference to a plasma concentration or a local concentration of the linear polyribonucleotide or antibody or antigen-binding fragment thereof.

The engineered polynucleotide, the engineered polypeptide the AAV vector comprising the engineered polynucleotide, the AAV comprising the engineered polynucleotide, the cell transduced with the AAV vector, the viral particle comprising the engineered polynucleotide, the pharmaceutical composition, or a combination thereof described herein may be in a unit dosage form suitable for a single administration of a precise dosage. In unit dosage form, the formulation may be divided into unit doses containing appropriate quantities of the compositions. In unit dosage form, the formulation may be divided into unit doses containing appropriate quantities of one or more linear polyribonucleotides, antibodies or the antigen-binding fragments thereof, and/or therapeutic agents. The unit dosage may be in the form of a package containing discrete quantities of the formulation. Non-limiting examples are packaged injectables, vials, and ampoules. An aqueous suspension composition disclosed herein may be packaged in a single-dose non-reclosable container. Multiple-dose reclosable containers may be used, for example, in combination with or without a preservative. A formulation for injection disclosed herein may be present in a unit dosage form, for example, in ampoules, or in multi dose containers with a preservative.

In some cases, an increased level of a biologic in a subject is at least a 5-fold, a 10-fold, a 20-fold, a 50-fold, a 100-fold, a 200-fold, or a 500-fold increased, as determined by a diagnostic assay.

Suitable diagnostic assays can include ocular diagnostic assays. Ocular diagnostic assays can include ophthalmic testing such as refraction testing, ocular scans, Ocular coherence tomography, Farnworth-Munsell 100 Hue Test, Computerized Optic Disc Imaging and Nerve Fiber Layer Analysis (GDX, HRT, OCT), Corneal Topography, Electroretinography (ERG), electro-oculography (EOG), visual evoked potentials (VEP), visual evoked response (VER), Fluorescein Angiography, Ocular Coherence Tomography (OCT), retinal photography, fundus photography, Specular Microscopy, Goldmann, Humphrey, FDT, Octopus, Biometry/IOL calculation, A-Scan, B-Scan, and combinations thereof.

In some cases, a retinal test can be utilized. Nonlimiting methods for assessing retinal function and changes thereof include assessing visual acuity (e.g. best-corrected visual acuity [BCVA], ambulation, navigation, object detection and discrimination), assessing visual field (e.g. static and kinetic visual field perimetry), performing a clinical examination (e.g. slit lamp examination of the anterior and posterior segments of the eye), assessing electrophysiological responsiveness to all wavelengths of light and dark (e.g. all forms of electroretinography (ERG) [full-field, multifocal and pattern], all forms of visual evoked potential (VEP), electrooculography (EOG), color vision, dark adaptation and/or contrast sensitivity). Nonlimiting methods for assessing anatomy and retinal health and changes thereof include Optical Coherence Tomography (OCT), fundus photography, adaptive optics scanning laser ophthalmoscopy (AO-SLO), fluorescence and/or autofluorescence; measuring ocular motility and eye movements (e.g. nystagmus, fixation preference, and stability), measuring reported outcomes (patient-reported changes in visual and non-visually-guided behaviors and activities, patient-reported outcomes [PRO], questionnaire-based assessments of quality-of-life, daily activities and measures of neurological function (e.g. functional Magnetic Resonance Imaging (MRI)).

In some embodiments, the engineered polynucleotide, the AAV vector comprising the engineered polynucleotide, the AAV comprising the engineered polynucleotide, the cell transduced with the AAV vector, the viral particle comprising the engineered polynucleotide, the pharmaceutical composition, or a combination thereof 20%, at least 30%, at least 40%, at least 50%, at least 60%, at least 70%, at least 80%, at least 90%, at least 100%, at least 200%, at least 500%, or more compared to a comparable cell that is not contacted with the engineered polynucleotide, the AAV vector comprising the engineered polynucleotide, the AAV comprising the engineered polynucleotide, or the pharmaceutical composition.

In some embodiments, the method of treatment described herein can treat an ocular disease. Relevant ocular diseases and conditions can include but are not limited to: blindness, Achromatopsia, Age-related macular degeneration (AMD), Diabetic retinopathy (DR), Glaucoma, Bardet-Biedl Syndrome, Best Disease, Choroideremia, Leber Congenital Amaurosis, Macular degeneration, Polypoidal choroidal vasculopathy (PCV), Retinitis pigmentosa, Refsum disease, Stargardt disease, Usher syndrome, X-linked retinoschisis (XLRS), Rod-cone dystrophy, Cone-rod dystrophy, Oguchi disease, Malattia Leventinese (Familial Dominant Drusen), and Blue-cone monochromacy. In an embodiment, the ocular disease or condition is AMD. AMD can be wet AMD or dry AMD.

In some cases, an administration of a pharmaceutical composition is sufficient to reduce at least a symptom of a disease or condition, treat the disease or condition, and/or eliminate the disease or condition. In some cases, improvements of diseases or conditions can be ascertained by any of the provided diagnostic assays. In other cases, an improvement can be obtained via an interview with the treated subject. For example, a subject may be able to communicate to an attending physician that their vision is improved as compared to their vision prior to administration of a subject pharmaceutical. In other cases, an in vivo animal model may be used to ascertain reduction of a disease or condition after treatment. Suitable animal models include mouse models, primate models, rat models, canine models, and the like.

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Any systems, methods, software, and platforms described herein are modular. Accordingly, terms such as “first” and “second” do not necessarily imply priority, order of importance, or order of acts.

The term “about” when referring to a number or a numerical range means that the number or numerical range referred to is an approximation within experimental variability (or within statistical experimental error), and the number or numerical range may vary from, for example, from 1% to 15% of the stated number or numerical range. In examples, the term “about” refers to +10% of a stated number or value.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “AAV,” “AAV construct,” or “recombinant AAV” or “AAV” refer to adeno-associated virus of any of the known serotypes, including AAV-1, AAV-2, AAV-3, AAV-4, AAV-5, AAV-6, AAV-7, AAV-8, AAV-9, AAV-10, AAV-11, AAV-12, AAV-13, or scAAV, rh10, chimeric or hybrid AAV, or any combination, derivative, or variant thereof. AAV is a small non-enveloped single-stranded DNA virus. They are non-pathogenic parvoviruses and can require helper viruses, such as adenovirus, herpes simplex virus, vaccinia virus, and CMV, for replication. Wild-type AAV is common in the general population, and is not associated with any known pathologies. A hybrid AAV is an AAV comprising a capsid protein of one AAV serotype and genomic material from another AAV serotype. A chimeric AAV comprises genetic and/or protein sequences derived from two or more AAV serotypes, and can include mutations made to the genetic sequences of those two or more AAV serotypes. An exemplary chimeric AAV can comprise a chimeric AAV capsid, for example, a capsid protein with one or more regions of amino acids derived from two or more AAV serotypes. An AAV variant is an AAV comprising one or more amino acid mutations in its genome or proteins as compared to its parental AAV, e.g., one or more amino acid mutations in its capsid protein as compared to its parental AAV. AAV, as used herein, includes avian AAV, bovine AAV, canine AAV, equine AAV, primate AAV, non-primate AAV, and ovine AAV, where primate AAV refers to AAV that infect non-primates, and where non-primate AAV refers to AAV that infect non-primate animals, such as avian AAV that infects avian animals. In some cases, the wild-type AAV contains rep and cap genes, where the rep gene is required for viral replication and the cap gene is required for the synthesis of capsid proteins. As used herein, the terms “recombinant AAV” and “rAAV” are interchangeable.

The terms “recombinant AAV vector” or “AAV vector” or “AAV vector” refer to a vector derived from any of the AAV serotypes mentioned above. In some cases, an AAV vector can comprise one or more of the AAV wild-type genes deleted in whole or part, such as the rep and/or cap genes, but contains functional elements that are required for packaging and use of AAV virus for gene therapy. For example, functional inverted terminal repeats or ITR sequences that flank an open reading frame or exogenous sequences cloned in are known to be important for replication and packaging of an AAV virion, but the ITR sequences can be modified from the wild-type nucleotide sequences, including insertions, deletions, or substitutions of nucleotides, so that the AAV is suitable for use for the embodiments described herein, such as a gene therapy or gene delivery system. In some aspects, a self-complementary vector (sc) can be used, such as a self-complementary AAV vector, which can bypass the requirement for viral second-strand DNA synthesis and can lead to higher rate of expression of a transgene protein. In some aspects, AAV vectors can be generated to allow selection of an optimal serotype, promoter, and transgene. In some cases, the vector can be targeted vector or a modified vector that selectively binds or infects immune cells.

The terms “AAV virion” or “AAV virion” refer to a virus particle comprising a capsid comprising at least one AAV capsid protein that encapsidates an AAV vector as described herein, where the vector can further comprise a heterologous nucleic acid sequence or a transgene in some embodiments. A virion can be an engineered virion.

The term “subject,” “host,” “individual,” and “patient” are as used interchangeably herein to refer to animals, typically mammalian animals. Any suitable mammal can be administered a composition as described herein (such as an engineered guide RNA) or treated by a method as described herein. A subject can be a vertebrate or an invertebrate. A subject can be a laboratory animal. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. In some embodiments a subject is a human. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

The term “protein”, “peptide”, and “polypeptide” are used interchangeably and in their broadest sense to refer to a compound of two or more subunit amino acids, amino acid analogs or peptidomimetics. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics. As used herein, the term “fusion protein” refers to a protein comprised of domains from more than one naturally occurring or recombinantly produced protein, where generally each domain serves a different function. In this regard, the term “linker” refers to a protein fragment that is used to link these domains together-optionally to preserve the conformation of the fused protein domains and/or prevent unfavorable interactions between the fused protein domains which may compromise their respective functions.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EMBODIMENTS

Embodiment 1: An engineered polynucleotide comprising one or more expression cassettes, the one or more expression cassettes encoding a first angiogenesis inhibitor and a second angiogenesis inhibitor.

Embodiment 2: An engineered polynucleotide comprising one or more expression cassettes, the one or more expression cassettes encoding: a complement 3 (C3) inhibitor or a C3 degraded fragment; and a natriuretic peptide.

Embodiment 3: The engineered polynucleotide of embodiment 2, wherein the complement 3 inhibitor or the C3 degraded fragment and the natriuretic peptide are covalently connected by a linker.

Embodiment 4: The engineered polynucleotide of embodiment 2 or 3, wherein the complement 3 inhibitor comprises an amino acid sequence that is at least 80% identical to any one of SEQ ID NOs: 1-15.

Embodiment 5: The engineered polynucleotide of any one of embodiments 2-4, wherein the complement 3 inhibitor comprises an amino acid sequence comprising any one of SEQ ID NOs: 1-15.

Embodiment 6: The engineered polynucleotide of any one of embodiments 2-5, wherein the complement 3 inhibitor is encoded by a nucleic acid sequence that is at least 80% identical to any one of SEQ ID NOs: 20-33.

Embodiment 7: The engineered polynucleotide of any one of embodiments 2-6, wherein the complement 3 inhibitor is encoded by a nucleic acid sequence comprising any one of SEQ ID NOs: 20-33.

Embodiment 8: The engineered polynucleotide of any one of embodiments 2-7, wherein the natriuretic peptide comprises a C-type natriuretic peptide (CNP).

Embodiment 9: The engineered polynucleotide of any one of embodiments 2-8, wherein the natriuretic peptide is covalently connected to an antibody or fragment thereof.

Embodiment 10: The engineered polynucleotide of embodiment 9, wherein the antibody or fragment thereof comprises a fragment crystallizable (Fc) region.

Embodiment 11: The engineered polynucleotide of any one of embodiments 8-10, wherein the natriuretic peptide comprises an amino acid sequence that is at least 80% identical to any one of SEQ ID NOs: 61-72.

Embodiment 12: The engineered polynucleotide of any one of embodiments 8-11, wherein the natriuretic peptide comprises an amino acid sequence that is any one of SEQ ID NOs: 61-72.

Embodiment 13: The engineered polynucleotide of embodiment 1, wherein the first angiogenesis inhibitor and the second angiogenesis inhibitor is each encoded by an expression cassette of the one or more expression cassettes.

Embodiment 14: The engineered polynucleotide of embodiments 1 or 13, wherein the first angiogenesis inhibitor and the second angiogenesis inhibitor are encoded by an expression cassette of the one or more expression cassettes.

Embodiment 15: The engineered polynucleotide of any one of embodiments 1 or 13-14, wherein the first angiogenesis inhibitor and the second angiogenesis inhibitor are operatively coupled.

Embodiment 16: The engineered polynucleotide of any one of embodiments 1 or 13-15, wherein the first angiogenesis inhibitor and the second angiogenesis inhibitor are covalently connected by a linker.

Embodiment 17: The engineered polynucleotide of any one of embodiments 1 or 13-16, wherein the first angiogenesis inhibitor comprises a complement inhibitor.

Embodiment 18: The engineered polynucleotide of any one of embodiments 1 or 13-17, wherein the complement inhibitor comprises a complement 3 inhibitor or a C3 degraded fragment.

Embodiment 19: The engineered polynucleotide of any one of embodiments 1 or 13-18, wherein the complement 3 inhibitor comprises an amino acid sequence that is at least 80% identical to any one of SEQ ID NOs: 1-15.

Embodiment 20: The engineered polynucleotide of any one of embodiments 1 or 13-19, wherein the complement 3 inhibitor comprises an amino acid sequence comprising any one of SEQ ID NOs: 1-15.

Embodiment 21: The engineered polynucleotide of any one of embodiments 1 or 13-20, wherein the complement 3 inhibitor is encoded by a nucleic acid sequence that is at least 80% identical to any one of SEQ ID NOs: 20-33.

Embodiment 22: The engineered polynucleotide of any one of embodiments 1 or 13-21, wherein the complement 3 inhibitor is encoded by a nucleic acid sequence comprising any one of SEQ ID NOs: 20-33.

Embodiment 23: The engineered polynucleotide of any one of embodiments 1 or 13-22, wherein the first angiogenesis inhibitor comprises an inhibitor of a membrane attack complex (MAC).

Embodiment 24: The engineered polynucleotide of any one of embodiments 1 or 13-23, wherein the inhibitor of the MAC comprises CD59.

Embodiment 25: The engineered polynucleotide of any one of embodiments 1 or 13-24, wherein the CD59 comprises an amino acid sequence that is at least 80% identical to any one of SEQ ID NOs: 41-45.

Embodiment 26: The engineered polynucleotide of any one of embodiments 1 or 13-25, wherein the CD59 comprises an amino acid sequence that is any one of SEQ ID NOs: 41-45.

Embodiment 27: The engineered polynucleotide of any one of embodiments 1 or 13-26, wherein the second angiogenesis inhibitor comprises an inhibitor of a membrane attack complex (MAC).

Embodiment 28: The engineered polynucleotide of any one of embodiments 1 or 13-27, wherein the inhibitor of the MAC comprises CD59.

Embodiment 29: The engineered polynucleotide of any one of embodiments 1 or 13-28, wherein the CD59 comprises an amino acid sequence that is at least 80% identical to any one of SEQ ID NOs: 41-45.

Embodiment 30: The engineered polynucleotide of any one of embodiments 1 or 13-29, wherein the CD59 comprises an amino acid sequence that is any one of SEQ ID NOs: 41-45.

Embodiment 31: The engineered polynucleotide of any one of embodiments 1 or 13-30, wherein the second angiogenesis inhibitor comprises a natriuretic peptide.

Embodiment 32: The engineered polynucleotide of embodiment 31, wherein the natriuretic peptide comprises a C-type natriuretic peptide (CNP).

Embodiment 33: The engineered polynucleotide of embodiments 31 or 32, wherein the natriuretic peptide is covalently connected to an antibody or fragment thereof.

Embodiment 34: The engineered polynucleotide of embodiment 33, wherein the antibody or fragment thereof comprises a fragment crystallizable (Fc) region.

Embodiment 35: The engineered polynucleotide of any one of embodiments 31-34, wherein the natriuretic peptide comprises an amino acid sequence that is at least 80% identical to any one of SEQ ID NOs: 61-72.

Embodiment 36: The engineered polynucleotide of any one of embodiments 31-35, wherein the natriuretic peptide comprises an amino acid sequence that is any one of SEQ ID NOs: 61-72.

Embodiment 37: The engineered polynucleotide of any one of embodiments 1, 13-36, wherein the second angiogenesis inhibitor comprises a collagen or fragment thereof.

Embodiment 38: The engineered polynucleotide of any one of embodiments 1, 13-37, wherein the second angiogenesis inhibitor comprises an endostatin or fragment thereof.

Embodiment 39: The engineered polynucleotide of any one of embodiments 1, 13-38, wherein the second angiogenesis inhibitor comprises an amino acid sequence that is at least 80% identical to SEQ ID NO: 51.

Embodiment 40: The engineered polynucleotide of any one of embodiments 1, 13-39, wherein the second angiogenesis inhibitor comprises the amino acid sequence that is SEQ ID NO: 51.

Embodiment 41: The engineered polynucleotide of any one of embodiments 1, 13-40, wherein the second angiogenesis inhibitor comprises a VEGF inhibitor.

Embodiment 42: The engineered polynucleotide of embodiment 41, wherein the VEGF inhibitor comprises an amino acid sequence that is at least 80% identical to any one of SEQ ID NOs: 81-92.

Embodiment 43: The engineered polynucleotide of embodiments 41 or 42, wherein the VEGF inhibitor comprises an amino acid sequence that is any one of SEQ ID NOs: 81-92.

Embodiment 44: The engineered polynucleotide of any one of embodiments 3-43, wherein the linker comprises a cleavable linker.

Embodiment 45: The engineered polynucleotide of embodiment 44, wherein the cleavable linker comprises a Furin protease linker.

Embodiment 46: The engineered polynucleotide of any one of the previous embodiments, further encoding a third angiogenesis inhibitor.

Embodiment 47: The engineered polynucleotide of any one of embodiments 2-46, wherein the C3 degraded fragment comprises C3a, C3b, iC3b, C3f, C3c, C3d, C3g, or a combination thereof.

Embodiment 48: The engineered polynucleotide of any one of the previous embodiments, wherein the engineered polynucleotide comprises a viral vector.

Embodiment 49: The engineered polynucleotide of embodiment 48, wherein the viral vector comprises an AAV vector.

Embodiment 50: The engineered polynucleotide of embodiment 49, wherein the AAV vector comprises an AAV serotype comprising AAV1, AAV2, AAV3, AAV4, AAV5, AAV6, AAV7, AAV8, AAV9, AAV10, AAV11, AAV12, or any combination thereof.

Embodiment 51: The engineered polynucleotide of embodiments 49 or 50, wherein the AAV vector is an AAV2 vector.

Embodiment 52: The engineered polynucleotide of any one of embodiments 49-51, wherein the AAV vector encodes an engineered AAV capsid.

Embodiment 53: The engineered polynucleotide of embodiment 52, wherein the engineered AAV capsid comprises an amino acid sequence of any one of SEQ ID NOs: 161-182 and SEQ ID NOs: 191-210.

Embodiment 54: The engineered polynucleotide of embodiments 52 or 53, wherein the engineered AAV capsid comprises the amino acid sequence of SEQ ID NO: 169.

Embodiment 55: The engineered polynucleotide of any one of embodiments 1-54, wherein the first angiogenesis inhibitor comprises: a complement 3 inhibitor or a C3 degraded fragment comprising a C3a, C3b, iC3b, C3f, C3c, C3d, C3g, or a combination thereof; and the second angiogenesis inhibitor comprises a CNP.

Embodiment 56: The engineered polynucleotide of any one of embodiments 1-55, wherein the first angiogenesis inhibitor comprises the complement 3 inhibitor, and the second angiogenesis inhibitor comprises a CNP36.

Embodiment 57: The engineered polynucleotide of any one of embodiments 1-56, wherein the first angiogenesis inhibitor comprises the complement 3 inhibitor, and the second angiogenesis inhibitor comprises an Fc-CNP36.

Embodiment 58: The engineered polynucleotide of any one of embodiments 1-57, further encoding a third angiogenesis inhibitor.

Embodiment 59: The engineered polynucleotide of embodiment 58, wherein the third angiogenesis inhibitor comprises an inhibitor of a membrane attack complex (MAC).

Embodiment 60: The engineered polynucleotide of embodiment 59, wherein the inhibitor of the MAC comprises a CD59.

Embodiment 61: The engineered polynucleotide of any one of embodiments 58-60, wherein the engineered polynucleotide encodes a protease site flanked by the second angiogenesis inhibitor and the third angiogenesis inhibitor.

Embodiment 62: The engineered polynucleotide of any one of embodiments 1-61, wherein the first angiogenesis inhibitor comprises an inhibitor of a CD59, and the second angiogenesis inhibitor comprises a CNP.

Embodiment 63: The engineered polynucleotide of any one of embodiments 1-62, wherein the first angiogenesis inhibitor comprises the CD59, and the second angiogenesis inhibitor comprises a CNP36.

Embodiment 64: The engineered polynucleotide of any one of embodiments 1-63, wherein the first angiogenesis inhibitor comprises the CD59, and the second angiogenesis inhibitor comprises an Fc-CNP36.

Embodiment 65: The engineered polynucleotide of any one of embodiments 1-64, wherein the first angiogenesis inhibitor comprises the CD59, and the second angiogenesis inhibitor comprises a complement 3 inhibitor fused to an Fc-CNP36.

Embodiment 66: The engineered polynucleotide of any one of embodiments 1-65, wherein the first angiogenesis inhibitor comprises a complement 3 inhibitor, and the second angiogenesis inhibitor comprises an endostatin.

Embodiment 67: The engineered polynucleotide of any one of embodiments 1-66, wherein the engineered polynucleotide encodes an Fc region flanked by the first angiogenesis inhibitor and the second angiogenesis inhibitor.

Embodiment 68: The engineered polynucleotide of any one of embodiments 1-67, wherein the first angiogenesis inhibitor comprises a VEGF inhibitor, and the second angiogenesis inhibitor comprises a complement 3 inhibitor.

Embodiment 69: The engineered polynucleotide of any one of embodiments 1-68, wherein the first angiogenesis inhibitor comprises a complement 3 inhibitor, and the second angiogenesis inhibitor comprises an Fc-CNP36, wherein the engineered polynucleotide further encodes a third angiogenesis inhibitor comprising a CD59.

Embodiment 70: The engineered polynucleotide of any one of embodiments 1-69, wherein the engineered polynucleotide further encodes a protease site flanked by the second angiogenesis inhibitor and the third angiogenesis inhibitor.

Embodiment 71: The engineered polynucleotide of any one of embodiments 1-70, wherein the protease site comprises a Furin protease site.

Embodiment 72: The engineered polynucleotide of any one of embodiments 1-71, wherein the first angiogenesis inhibitor comprises a complement 3 inhibitor, and the second angiogenesis inhibitor comprises an Fc-CNP36, wherein the engineered polynucleotide further encodes a third angiogenesis inhibitor comprising a CD59.

Embodiment 73: The engineered polynucleotide of any one of embodiments 1-72, wherein the first angiogenesis inhibitor comprises a CD59, and the second angiogenesis inhibitor comprises a complement 3 inhibitor, wherein the engineered polynucleotide further encodes a third angiogenesis inhibitor comprising a Fc-CNP36.

Embodiment 74: The engineered polynucleotide of any one of embodiments 1-73, wherein the first angiogenesis inhibitor comprises a CD59, and the second angiogenesis inhibitor comprises an endostatin, wherein the engineered polynucleotide further encodes a third angiogenesis inhibitor comprising a complement 3 inhibitor.

Embodiment 75: The engineered polynucleotide of any one of embodiments 1-74, wherein the first angiogenesis inhibitor comprises a CD59, and the second angiogenesis inhibitor comprises a VEGF inhibitor, wherein the engineered polynucleotide further encodes a third angiogenesis inhibitor comprising a complement 3 inhibitor.

Embodiment 76: The engineered polynucleotide of any one of embodiments 1-75, wherein the first angiogenesis inhibitor comprises a complement 3 inhibitor, and the second angiogenesis inhibitor comprises an endostatin, wherein the engineered polynucleotide further encodes a third angiogenesis inhibitor comprising a CD59.

Embodiment 77: The engineered polynucleotide of any one of embodiments 1-76, wherein the first angiogenesis inhibitor, the second angiogenesis inhibitor, and the third angiogenesis inhibitor is not a VEGF inhibitor.

Embodiment 78: The engineered polynucleotide of any one of embodiments 1-77, wherein the first angiogenesis inhibitor and the second angiogenesis inhibitor, upon administered to a subject, inhibits neovascularization in the subject.

Embodiment 79: The engineered polynucleotide of any one of embodiments 1-78, wherein the first angiogenesis inhibitor or the second angiogenesis inhibitor, upon administered to the subject, exhibits decreased inhibition of neovascularization in the subject compared to inhibition of neovascularization caused by a VEGF inhibitor.

Embodiment 80: An engineered polypeptide comprising a first angiogenesis inhibitor and a second angiogenesis inhibitor.

Embodiment 81: The engineered polypeptide of embodiment 80, wherein the first angiogenesis inhibitor and the second angiogenesis inhibitor are covalently connected by a linker.

Embodiment 82: The engineered polypeptide of any one of embodiments 80-81, wherein the first angiogenesis inhibitor comprises a complement inhibitor.

Embodiment 83: The engineered polypeptide of any one of embodiments 80-82, wherein the complement inhibitor comprises a complement 3 inhibitor or a C3 degraded fragment comprising a C3a, C3b, iC3b, C3f, C3c, C3d, C3g, or a combination thereof.

Embodiment 84: The engineered polypeptide of any one of embodiments 80-83, wherein the complement 3 inhibitor comprises an amino acid sequence that is at least 80% identical to any one of SEQ ID NOs: 1-15.

Embodiment 85: The engineered polypeptide of any one of embodiments 80-84, wherein the complement 3 inhibitor comprises an amino acid sequence comprising any one of SEQ ID NOs: 1-15.

Embodiment 86: The engineered polypeptide of any one of embodiments 80-85, wherein the complement 3 inhibitor is encoded by a nucleic acid sequence that is at least 80% identical to any one of SEQ ID NOs: 20-33.

Embodiment 87: The engineered polypeptide of any one of embodiments 80-86, wherein the complement 3 inhibitor is encoded by a nucleic acid sequence comprising any one of SEQ ID NOs: 20-33.

Embodiment 88: The engineered polypeptide of any one of embodiments 80-87, wherein the first angiogenesis inhibitor comprises an inhibitor of a membrane attack complex (MAC).

Embodiment 89: The engineered polypeptide of any one of embodiments 80-88, wherein the inhibitor of the MAC comprises CD59.

Embodiment 90: The engineered polypeptide of any one of embodiments 80-89, wherein the CD59 comprises an amino acid sequence that is at least 80% identical to any one of SEQ ID NOs: 41-45.

Embodiment 91: The engineered polypeptide of any one of embodiments 80-90, wherein the CD59 comprises an amino acid sequence that is any one of SEQ ID NOs: 41-45.

Embodiment 92: The engineered polypeptide of any one of embodiments 80-91, wherein the second angiogenesis inhibitor comprises a natriuretic peptide.

Embodiment 93: The engineered polypeptide of any one of embodiments 80-92, wherein the natriuretic peptide is covalently connected to an antibody or fragment thereof.

Embodiment 94: The engineered polypeptide of any one of embodiments 80-93, wherein the antibody or fragment thereof comprises a fragment crystallizable (Fc) region.

Embodiment 95: The engineered polypeptide of any one of embodiments 80-94, wherein the natriuretic peptide comprises an amino acid sequence that is at least 80% identical to any one of SEQ ID NOs: 61-72.

Embodiment 96: The engineered polypeptide of any one of embodiments 80-95, wherein the natriuretic peptide comprises an amino acid sequence that is any one of SEQ ID NOs: 61-72.

Embodiment 97: The engineered polypeptide of any one of embodiments 80-96, wherein the second angiogenesis inhibitor comprises a collagen or fragment thereof.

Embodiment 98: The engineered polypeptide of any one of embodiments 80-97, wherein the second angiogenesis inhibitor comprises an endostatin or fragment thereof.

Embodiment 99: The engineered polypeptide of any one of embodiments 80-98, wherein the second angiogenesis inhibitor comprises an amino acid sequence that is at least 80% identical to SEQ ID NO: 51.

Embodiment 100: The engineered polypeptide of any one of embodiments 80-99, wherein the second angiogenesis inhibitor comprises the amino acid sequence that is SEQ ID NO: 51.

Embodiment 101: The engineered polypeptide of any one of embodiments 80-100, wherein the second angiogenesis inhibitor comprises a VEGF inhibitor.

Embodiment 102: The engineered polypeptide of any one of embodiments 80-101, wherein the VEGF inhibitor comprises an amino acid sequence that is at least 80% identical to any one of SEQ ID NOs: 81-92.

Embodiment 103: The engineered polypeptide of any one of embodiments 80-102, wherein the VEGF inhibitor comprises an amino acid sequence that is any one of SEQ ID NOs: 81-92.

Embodiment 104: The engineered polypeptide of any one of embodiments 80-103, wherein the linker comprises a cleavable linker.

Embodiment 105: The engineered polypeptide of any one of embodiments 80-104, wherein the cleavable linker comprises a Furin protease linker.

Embodiment 106: The engineered polypeptide of any one of the previous embodiments 80-105, further encoding a third angiogenesis inhibitor.

Embodiment 107: The engineered polypeptide of embodiment 106, wherein the third angiogenesis inhibitor is different from the first angiogenesis inhibitor and the second angiogenesis inhibitor.

Embodiment 108: A vector comprising the engineered polynucleotide of any one of embodiments 1-79.

Embodiment 109: A vector encoding the engineered polypeptide of any one of embodiments 80-107.

Embodiment 110: The vector of embodiments 108 or 109, wherein the vector encodes an AAV capsid.

Embodiment 111: The vector of any one of embodiments 108-110, wherein the AAV capsid comprises an engineered AAV capsid.

Embodiment 112: The vector of any one of embodiments 108-111, wherein the modified AAV capsid comprises a engineered AAV2 capsid.

Embodiment 113: A viral particle comprising the engineered polynucleotide of any one of embodiments 1-79 or the vector of any one of embodiments 108-112.

Embodiment 114: The viral particle of embodiment 113, wherein the viral particle comprises an AAV capsid.

Embodiment 115: The viral particle of any one of embodiments 113-114, wherein the AAV capsid comprises an engineered AAV capsid.

Embodiment 116: The viral particle of any one of embodiments 113-115, wherein the modified AAV capsid comprises a engineered AAV2 capsid.

Embodiment 117: A cell comprising the engineered polynucleotide of any one of embodiments 1-79, the engineered polypeptide of any one of embodiments 80-107, the vector of any one of embodiments 108-112, or the viral particle of any one of embodiments 113-116.

Embodiment 118: A composition comprising: a complement 3 inhibitor or a C3 degraded fragment comprising a C3a, C3b, iC3b, C3f, C3c, C3d, C3g, or a combination thereof; and a natriuretic peptide.

Embodiment 119: The composition of embodiment 118, wherein the natriuretic peptide comprises a C-type natriuretic peptide (CNP).

Embodiment 120: The composition of any one of embodiments 118-119, wherein the natriuretic peptide is covalently connected to an antibody or fragment thereof.

Embodiment 121: The composition of any one of embodiments 118-120, wherein the antibody or fragment thereof comprises a fragment crystallizable (Fc) region.

Embodiment 122: The composition of any one of embodiments 118-121, wherein the natriuretic peptide comprises an amino acid sequence that is at least 80% identical to any one of SEQ ID NOs: 61-72.

Embodiment 123: The composition of any one of embodiments 118-122, wherein the natriuretic peptide comprises an amino acid sequence that is any one of SEQ ID NOs: 61-72.

Embodiment 124: The composition of any one of embodiments 118-123, further comprising a CD59, an endostatin, a VEGF inhibitor, or a combination thereof.

Embodiment 125: A composition comprising: a complement 3 inhibitor or a C3 degraded fragment comprising a C3a, C3b, iC3b, C3f, C3c, C3d, C3g, or a combination thereof; and an inhibitor of a membrane attack complex (MAC).

Embodiment 126: The composition of any one of embodiments 118-125, wherein the inhibitor of the MAC comprises CD59.

Embodiment 127: The composition of any one of embodiments 118-126, wherein the CD59 comprises an amino acid sequence that is at least 80% identical to any one of SEQ ID NOs: 41-45.

Embodiment 128: The composition of any one of embodiments 118-127, wherein the CD59 comprises an amino acid sequence that is any one of SEQ ID NOs: 41-45.

Embodiment 129: The composition of any one of embodiments 118-128, further comprising a natriuretic peptide, an endostatin, a VEGF inhibitor, or a combination thereof.

Embodiment 130: A composition comprising: a complement 3 inhibitor or a C3 degraded fragment comprising a C3a, C3b, iC3b, C3f, C3c, C3d, C3g, or a combination thereof; and a collagen or fragment thereof.

Embodiment 131: The composition of any one of embodiments 118-130, wherein the collagen or fragment thereof comprises an endostatin or fragment thereof.

Embodiment 132: The composition of any one of embodiments 118-131, wherein the endostatin or fragment thereof comprises an amino acid sequence that is at least 80% identical to SEQ ID NO: 51.

Embodiment 133: The composition of any one of embodiments 118-132, wherein the endostatin or fragment thereof comprises the amino acid sequence that is SEQ ID NO: 51.

Embodiment 134: The composition of any one of embodiments 118-133, further comprising a natriuretic peptide, a CD59, a VEGF inhibitor, or a combination thereof.

Embodiment 135: A composition comprising: a complement 3 inhibitor or a C3 degraded fragment comprising a C3a, C3b, iC3b, C3f, C3c, C3d, C3g, or a combination thereof, and a VEGF inhibitor.

Embodiment 136: The composition of any one of embodiments 118-135, wherein the VEGF inhibitor comprises an amino acid sequence that is at least 80% identical to any one of SEQ ID NOs: 81-92.

Embodiment 137: The composition of any one of embodiments 118-136, wherein the VEGF inhibitor comprises an amino acid sequence that is any one of SEQ ID NOs: 81-92.

Embodiment 138: The composition of any one of embodiments 118-137, further comprising a natriuretic peptide, a CD59, an endostatin, or a combination thereof.

Embodiment 139: The composition of any one of embodiments 118-138, wherein the complement 3 inhibitor comprises an amino acid sequence that is at least 80% identical to any one of SEQ ID NOs: 1-15.

Embodiment 140: The composition of any one of embodiments 118-139, wherein the complement 3 inhibitor comprises an amino acid sequence comprising any one of SEQ ID NOS: 1-15.

Embodiment 141: The composition of any one of embodiments 118-140, wherein the complement 3 inhibitor is encoded by a nucleic acid sequence that is at least 80% identical to any one of SEQ ID NOs: 20-33.

Embodiment 142: The composition of any one of embodiments 118-141, wherein the complement 3 inhibitor is encoded by a nucleic acid sequence comprising any one of SEQ ID NOs: 20-33.

Embodiment 143: A composition comprising a CD59 and a natriuretic peptide.

Embodiment 144: The composition of any one of embodiments 118-143, wherein the natriuretic peptide comprises a C-type natriuretic peptide (CNP).

Embodiment 145: The composition of any one of embodiments 118-144, wherein the natriuretic peptide is covalently connected to an antibody or fragment thereof.

Embodiment 146: The composition of any one of embodiments 118-145, wherein the antibody or fragment thereof comprises a fragment crystallizable (Fc) region.

Embodiment 147: The composition of any one of embodiments 118-146, wherein the natriuretic peptide comprises an amino acid sequence that is at least 80% identical to any one of SEQ ID NOs: 61-72.

Embodiment 148: The composition of any one of embodiments 118-147, wherein the natriuretic peptide comprises an amino acid sequence that is any one of SEQ ID NOs: 61-72.

Embodiment 149: The composition of any one of embodiments 143-148, further comprising a complement 3 inhibitor, an endostatin, a VEGF inhibitor, or a combination thereof.

Embodiment 150: A composition comprising a CD59 and a collagen or fragment thereof.

Embodiment 151: The composition of any one of embodiments 118-150, wherein the collagen or fragment thereof comprises an endostatin or fragment thereof.

Embodiment 152: The composition of any one of embodiments 118-151, wherein the endostatin or fragment thereof comprises an amino acid sequence that is at least 80% identical to SEQ ID NO: 51.

Embodiment 153: The composition of any one of embodiments 118-152, wherein the endostatin or fragment thereof comprises the amino acid sequence that is SEQ ID NO: 51.

Embodiment 154: The composition of any one of embodiments 118-153, further comprising a natriuretic peptide, a complement 3 inhibitor, a VEGF inhibitor, or a combination thereof.

Embodiment 155: A composition comprising a CD59 and a VEGF inhibitor.

Embodiment 156: The composition of any one of embodiments 118-155, wherein the VEGF inhibitor comprises an amino acid sequence that is at least 80% identical to any one of SEQ ID NOs: 81-92.

Embodiment 157: The composition of any one of embodiments 118-156, wherein the VEGF inhibitor comprises an amino acid sequence that is any one of SEQ ID NOs: 81-92.

Embodiment 158: The composition of any one of embodiments 118-157, further comprising a natriuretic peptide, a complement 3 inhibitor, an endostatin, or a combination thereof.

Embodiment 159: The composition of any one of embodiments 118-158, wherein the CD59 comprises an amino acid sequence that is at least 80% identical to any one of SEQ ID NOs: 41-45.

Embodiment 160: The composition of any one of embodiments 118-159, wherein the CD59 comprises an amino acid sequence that is any one of SEQ ID NOs: 41-45.

Embodiment 161: A pharmaceutical composition comprising the engineered polynucleotide of any one of embodiments 1-79, the engineered polypeptide of any one of embodiments 80-107, the vector of any one of embodiments 108-112, the viral particle of any one of embodiments 113-116, the cell of embodiment 117, or the composition of any one of embodiments 118-160.

Embodiment 162: The pharmaceutical composition of embodiment 161, wherein the pharmaceutical composition is formulated for administering intrathecally, intraocularly, intravitreally, retinally, intravenously, intramuscularly, intraventricularly, intracerebrally, intracerebellarly, intracerebroventricularly, intraperenchymally, subcutaneously, subretinally, suprachoroidally, intratumorally, pulmonarily, endotracheally, intraperitoneally, intravesically, intravaginally, intrarectally, orally, sublingually, transdermally, by inhalation, by inhaled nebulized form, by intraluminal-GI route, or a combination thereof to a subject in need thereof.

Embodiment 163: The pharmaceutical composition of any one of embodiments 160-162, wherein the pharmaceutical composition is formulated for administering intravitreally, subretinally, or suprachoroidally.

Embodiment 164: A method comprising contacting a cell obtained from a subject the engineered polynucleotide of any one of embodiments 1-79, the engineered polypeptide of any one of embodiments 80-107, the vector of any one of embodiments 108-112, the viral particle of any one of embodiments 113-116, the cell of embodiment 117, the composition of any one of embodiments 118-160, or the pharmaceutical composition of any one of embodiments 161-163.

Embodiment 165: A method of treating a disease or condition in a subject, comprising: administering to the subject the engineered polynucleotide of any one of embodiments 1-79, the engineered polypeptide of any one of embodiments 80-107, the vector of any one of embodiments 108-112, the viral particle of any one of embodiments 113-116, the cell of embodiment 117, the composition of any one of embodiments 118-160, or the pharmaceutical composition of any one of embodiments 161-163.

Embodiment 166: The method of any one of embodiments 164 or 165, wherein once of the administering is curative of the disease or condition.

Embodiment 167: The method of any one of embodiments 164-166, wherein the administering does not comprise daily administration.

Embodiment 168: The method of any one of embodiments 164-167, wherein the administering comprises a weekly administration, a bi-weekly administration, a monthly administration, a bi-month administration, a semiannual administration, an annual administration, or a combination thereof.

Embodiment 169: The method of any one of embodiments 164-168, wherein the disease or condition comprises an ocular disease.

Embodiment 170: The method of any one of embodiments 164-169, wherein the ocular disease comprises ocular ischemic syndrome, proliferative retinopathies, neovascular glaucoma (NG), glaucoma, traumatic glaucoma, uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

Embodiment 171: The method of any one of embodiments 164-170, wherein the ocular disease comprises GA or dAMD.

Embodiment 172: A method of treating a disease or condition in a subject, the method comprising administering to the subject an engineered polynucleotide comprising one or more expression cassettes, the one or more expression cassettes encoding a first angiogenesis inhibitor and a second angiogenesis inhibitor.

Embodiment 173: The method of any one of embodiments 164-172, wherein the first angiogenesis inhibitor and the second angiogenesis inhibitor is each encoded by an expression cassette of the one or more expression cassettes.

Embodiment 174: The method of any one of embodiments 164-173, wherein the first angiogenesis inhibitor and the second angiogenesis inhibitor are operatively coupled.

Embodiment 175: The method of any one of embodiments 164-174, wherein the first angiogenesis inhibitor and the second angiogenesis inhibitor are covalently connected by a linker.

Embodiment 176: The method of any one of embodiments 164-175, wherein the first angiogenesis inhibitor comprises a complement inhibitor.

Embodiment 177: The method of any one of embodiments 164-176, wherein the complement inhibitor comprises a complement 3 inhibitor or a C3 degraded fragment comprising a C3a, C3b, iC3b, C3f, C3c, C3d, C3g, or a combination thereof.

Embodiment 178: The method of any one of embodiments 164-177, wherein the complement 3 inhibitor comprises an amino acid sequence that is at least 80% identical to any one of SEQ ID NOs: 1-15.

Embodiment 179: The method of any one of embodiments 164-178, wherein the complement 3 inhibitor comprises an amino acid sequence comprising any one of SEQ ID NOS: 1-15.

Embodiment 180: The method of any one of embodiments 164-179, wherein the complement 3 inhibitor is encoded by a nucleic acid sequence that is at least 80% identical to any one of SEQ ID NOs: 20-33.

Embodiment 181: The method of any one of embodiments 164-180, wherein the complement 3 inhibitor is encoded by a nucleic acid sequence comprising any one of SEQ ID NOs: 20-33.

Embodiment 182: The method of any one of embodiments 164-181, wherein the first angiogenesis inhibitor comprises an inhibitor of a membrane attack complex (MAC).

Embodiment 183: The method of any one of embodiments 164-182, wherein the inhibitor of the MAC comprises CD59.

Embodiment 184: The method of any one of embodiments 164-183, wherein the CD59 comprises an amino acid sequence that is at least 80% identical to any one of SEQ ID NOs: 41-45.

Embodiment 185: The method of any one of embodiments 164-184, wherein the CD59 comprises an amino acid sequence that is any one of SEQ ID NOs: 41-45.

Embodiment 186: The method of any one of embodiments 164-185, wherein the second angiogenesis inhibitor comprises a natriuretic peptide.

Embodiment 187: The method of any one of embodiments 164-186, wherein the natriuretic peptide is covalently connected to an antibody or fragment thereof.

Embodiment 188: The method of any one of embodiments 164-187, wherein the antibody or fragment thereof comprises a fragment crystallizable (Fc) region.

Embodiment 189: The method of any one of embodiments 164-188, wherein the natriuretic peptide comprises an amino acid sequence that is at least 80% identical to any one of SEQ ID NOs: 61-72.

Embodiment 190: The method of any one of embodiments 164-189, wherein the natriuretic peptide comprises an amino acid sequence that is any one of SEQ ID NOs: 61-72.

Embodiment 191: The method of any one of embodiments 164-190, wherein the second angiogenesis inhibitor comprises a collagen or fragment thereof.

Embodiment 192: The method of any one of embodiments 164-191, wherein the second angiogenesis inhibitor comprises an endostatin or fragment thereof.

Embodiment 193: The method of any one of embodiments 164-192, wherein the second angiogenesis inhibitor comprises an amino acid sequence that is at least 80% identical to SEQ ID NO: 51.

Embodiment 194: The method of any one of embodiments 164-193, wherein the second angiogenesis inhibitor comprises the amino acid sequence that is SEQ ID NO: 51.

Embodiment 195: The method of any one of embodiments 164-194, wherein the second angiogenesis inhibitor comprises a VEGF inhibitor.

Embodiment 196: The method of any one of embodiments 164-195, wherein the VEGF inhibitor comprises an amino acid sequence that is at least 80% identical to any one of SEQ ID NOs: 81-92.

Embodiment 197: The method of any one of embodiments 164-196, wherein the VEGF inhibitor comprises an amino acid sequence that is any one of SEQ ID NOs: 81-92.

Embodiment 198: The method of any one of embodiments 164-197, wherein the linker comprises a cleavable linker.

Embodiment 199: The method of any one of embodiments 164-198, wherein the cleavable linker comprises a Furin protease linker.

Embodiment 200: The method of any one of embodiments 164-199, wherein the engineered polynucleotide further encodes a third angiogenesis inhibitor.

Embodiment 201: The method of any one of embodiments 164-200, wherein the third angiogenesis inhibitor is different from the first angiogenesis inhibitor and the second angiogenesis inhibitor.

Embodiment 202: The method of any one of embodiments 164-201, wherein the engineered polynucleotide comprises a viral vector.

Embodiment 203: The method of any one of embodiments 164-202, wherein the viral vector comprises an AAV vector.

Embodiment 204: The method of any one of embodiments 164-203, wherein the AAV vector comprises an AAV serotype comprising AAV1, AAV2, AAV3, AAV4, AAV5, AAV6, AAV7, AAV8, AAV9, AAV10, AAV11, AAV12, or any combination thereof.

Embodiment 205: The method of any one of embodiments 164-204, wherein the AAV vector is an AAV2 vector.

Embodiment 206: The method of any one of embodiments 164-205, wherein the AAV vector encodes an engineered AAV capsid.

Embodiment 207: The method of any one of embodiments 164-206, wherein the engineered AAV capsid comprises an amino acid sequence of any one of SEQ ID NOs: 161-182 and SEQ ID NOs: 191-210.

Embodiment 208: The method of any one of embodiments 164-207, wherein the engineered AAV capsid comprises the amino acid sequence of SEQ ID NO: 169.

Embodiment 209: The method of any one of embodiments 164-208, wherein once of the administering is curative of the disease or condition.

Embodiment 210: The method of any one of embodiments 164-209, wherein the administering does not comprise daily administration.

Embodiment 211: The method of any one of embodiments 164-210, wherein the administering comprises a weekly administration, a bi-weekly administration, a monthly administration, a bi-month administration, a semiannual administration, an annual administration, or a combination thereof.

Embodiment 212: The method of any one of embodiments 164-211, wherein the disease or condition comprises an ocular disease.

Embodiment 213: The method of any one of embodiments 164-212, wherein the ocular disease comprises ocular ischemic syndrome, proliferative retinopathies, neovascular glaucoma (NG), glaucoma, traumatic glaucoma, uveitis, neovascular uveitis, achromatopsia, age-related macular degeneration (nAMD), geographic atrophy (GA), dry age-related macular degeneration (dAMD), diabetic macular edema (DME), diabetic macular retinopathy (DMR), retinal vein occlusion (RVO), Bardet-Biedl Syndrome, Best Disease, choroideremia, Leber Congenital Amaurosis, macular degeneration, polypoidal choroidal vasculopathy (PCV), retinitis pigmentosa, Refsum disease, Stargardt disease, Usher syndrome, X-linked retinoschisis (XLRS), rod-cone dystrophy, Cone-rod dystrophy, Oguchi disease, Malattia leventinese (Familial Dominant Drusen), blue-cone monochromacy, or a combination thereof.

Embodiment 214: The method of any one of embodiments 164-213, wherein the ocular disease comprises GA or dAMD.

Embodiment 215: The method of any one of embodiments 164-214, wherein the first angiogenesis inhibitor comprises a complement 3 inhibitor, and the second angiogenesis inhibitor comprises an endostatin, wherein the engineered polynucleotide further encodes a third angiogenesis inhibitor comprising a CD59.

Embodiment 216: The method of any one of embodiments 164-215, wherein the first angiogenesis inhibitor, the second angiogenesis inhibitor, and the third angiogenesis inhibitor is not a VEGF inhibitor.

Embodiment 217: The method of any one of embodiments 164-216, wherein the first angiogenesis inhibitor and the second angiogenesis inhibitor, upon administered to a subject, inhibits neovascularization in the subject.

Embodiment 218: The method of any one of embodiments 164-217, wherein the first angiogenesis inhibitor or the second angiogenesis inhibitor, upon administered to the subject, exhibits decreased inhibition of neovascularization in the subject compared to inhibition of neovascularization caused by a VEGF inhibitor.

### EXAMPLES

The following illustrative examples are representative of embodiments of the stimulation, systems, and methods described herein and are not meant to be limiting in any way.

### Example 1. Vector for Expressing Multiple Angiogenesis Inhibitors

A vector can be designed to express multiple angiogenesis inhibitors described herein. FIG. 1 illustrates a vector map (top) for expression a complement 3 inhibitor (C3i) operatively coupled with a lead sequence (LS). The bottom vector map of FIG. 1 illustrates a complement 3 inhibitor (C3i) operatively coupled to a human IgG Fc region. The complement 3 inhibitor (C3i) can be modified to include at least one amino acid substitution compared to a wild-type complement 3 inhibitor (C3i). Table 1 illustrates exemplary C3i amino acid sequence that can be encoded by the vector described herein. Table 2 illustrates exemplary nucleic acid sequences for encoding the exemplary C3i.

TABLE 1
Exemplary C3i amino acid sequence

| Vector | Amino acid | SEQ | |
|---|---|---|---|
| name | sequence | ID NO | Note |
| Vector GGG | GICVWQDWGAHRCTN | 2 | C3i 15-mer |
| Vector KTP | GICVWQDWGAHRCAN | 3 | T14A (N15) |
| Vector KTQ | GYICVWQDWGAHRCTN | 4 | +2Y (N15) |
| Vector KTR | GYICVWQDWGAHRCAN | 5 | +2Y; T14A |
| | | | (N15) |
| Vector KAT | GICVWQDWGAHRCT | 6 | −N15 |
| Vector KAA | GICVWQDWGAHRCA | 7 | T14A; −N15 |
| Vector KAC | GYICVWQDWGAHRCT | 8 | +2Y; −N15 |
| Vector KAE | GYICVWQDWGAHRCA | 9 | +2Y; T14A; |
| | | | −N15 |
| Vector KAG | GICVWQDWGAHRCTQ | 10 | N15Q |
| Vector KAK | GICVWQDWGAHRCAQ | 11 | T14A; N15Q |
| Vector KAM | GYICVWQDWGAHRCTQ | 12 | +2Y; N15Q |
| Vector KAP | GYICVWQDWGAHRCAQ | 13 | +2Y; T14A; |
| | | | N15Q |

TABLE 2
Exemplary nucleic acid sequence encoding C3i

| Vector | | SEQ | |
|---|---|---|---|
| name | Nucleic acid sequence | ID NO | Note |
| Vector | ggcatctgcgtgtggcaggac | 22 | C3i 15-mer |
| GGG | tggggcgcccaccgctgcacc | | |
| | aac | | |
| Vector | ggcatctgcgtgtggcaggac | 23 | T14A (N15) |
| KTP | tggggcgcccaccgctgcGcc | | |
| | aac | | |
| Vector | ggcTACatctgcgtgtggcag | 24 | +2Y (N15) |
| KTQ | gactggggcgcccaccgctgc | | |
| | accaac | | |
| Vector | ggcTACatctgcgtgtggcag | 25 | +2Y; T14A |
| KTR | gactggggcgcccaccgctgc | | (N15) |
| | Gccaac | | |
| Vector | ggcatctgcgtgtggcaggac | 26 | −N15 |
| KAT | tggggcgcccaccgctgcacc | | |
| Vector | ggcatctgcgtgtggcaggac | 27 | T14A; −N15 |
| KAA | tggggcgcccaccgctgcGcc | | |
| Vector | ggcTACatctgcgtgtggcag | 28 | +2Y; −N15 |
| KAC | gactggggcgcccaccgctgc | | |
| | acc | | |
| Vector | ggcTACatctgcgtgtggcag | 29 | +2Y; T14A; |
| KAE | gactggggcgcccaccgctgc | | −N15 |
| | Gcc | | |
| Vector | ggcatctgcgtgtggcaggac | 30 | N15Q |
| KAG | tggggcgcccaccgctgcacc | | |
| | CAG | | |
| Vector | ggcatctgcgtgtggcaggac | 31 | T14A; N15Q |
| KAK | tggggcgcccaccgctgcGcc | | |
| | CAG | | |
| Vector | ggcTACatctgcgtgtggcag | 32 | +2Y; N15Q |
| KAM | gactggggcgcccaccgctgc | | |
| | accCAG | | |
| Vector | ggcTACatctgcgtgtggcag | 33 | +2Y; T14A; |
| KAP | gactggggcgcccaccgctgc | | N15Q |
| | GccCAG | | |

FIGS. 2A-B illustrate a complement 3 inhibitor (C3i) operatively coupled to a natriuretic polypeptide (e.g., an CNP36) by a human IgG Fc region (e.g., an Vector GAM or Vector GGE construct described herein). As shown in FIG. 2A, 15 mer-C3i is cloned between heavy chain leader sequence and Fc4 with the link of 2×GGGS in Vector CPE (SEQ ID NO: 15). FIG. 2B illustrates a similar vector, where the C3i further comprises an EVQL peptide or a DK peptide at the N-terminus of the C3i. FIG. 3 illustrates a vector for expressing an CD59, an C3i, and a natriuretic polypeptide (CNP36). The C3i as depicted in FIG. 3 can be any one of the C3i described herein, include the C3i as shown in FIG. 2B. FIG. 4 illustrates additional exemplary vectors for expressing the angiogenesis inhibitors described herein. Table 3 illustrates exemplary clone identification and construct name of the vectors described herein.

TABLE 3
Exemplary vector constructs

| | | |
|---|---|---|
| Clone ID | Vector name | AAV Vector |

Mono GOI

| | | |
|---|---|---|
| | AAV2.N54 -sham | |
| Vector GKE | AAV2.N54 -C3i | |
| Vector GEM | AAV2.N54 -sCD59 | SEQ ID NO: 169-Vector |
| | | GEM |
| Vector GTP | AAV2.N54-mCD59 | SEQ ID NO: 169-Vector |
| | | GTP |
| Vector GGG | AAV2.N54-Fc4-C3i | Vector GGG |
| Vector KTP | AAV2.N54-Fc-C3i, T14A | |
| Vector KTQ | AAV2.N54-Fc-C3i, +2Y | |
| Vector KTR | AAV2.N54-Fc-C3i, +2Y, T14A | |
| Vector KAT | AAV2.N54-Fc-C3i, −N15 | |
| Vector KAA | AAV2.N54-Fc-C3i, T14A, −N15 | |
| Vector KAC | AAV2.N54-Fc-C3i, +2Y, −N15 | |
| Vector KAE | AAV2.N54-Fc-C3i, +2Y, T14A, −N15 | |
| Vector KAG | AAV2.N54-Fc-C3i, N15Q | |
| Vector KAK | AAV2.N54-Fc-C3i, T14A, N15Q | |
| Vector KAM | AAV2.N54-Fc-C3i, +2Y, N15Q | |
| Vector KAP | AAV2.N54-Fc-C3i, +2Y, T14A, N15Q | |
| Vector KGQ, | AAV2.N54-sCD59 | |
| Vector KGR, | | |
| Vector KKT, | | |
| Vector KKA, | | |
| Vector KMR, | | |
| Vector KPT, | | |
| Vector KPC, | | |
| Vector KPP | | |
| Vector KGQ, | AAV2.N54-Fc4-C3i(N15Q) | |
| Vector KGR | | |
| Vector KRC | Vh(DK) sCD59 | |
| Vector KRE | vsCD59-6 × His | |
| Vector KRG | vsCD59 N18Q-6 × His | |
| Vector KRK | vsCD59 Q34E-6 × His | |
| Vector KRM | vsCD59 K38R-6 × His | |
| Vector KRP | vsCD59 Q34E, K38R-6 × His | |
| Vector KRQ | vsCD59 N18Q, K38R-6 × His | |
| Vector KRR | vsCD59 N18Q, Q33E, K38R-6 × His | |

Dual GOI

| | | |
|---|---|---|
| | AAV2.N54-C3i-Fc4-CNP36 (sham) | |
| Vector GAM | AAV2.N54-C3i-Fc4-CNP36 (AAV) | SEQ ID NO: 169-Vector |
| | | GAM |
| | AAV2.N54-sCD59-Fc4-CNP36-sham | |
| Vector GGM | AAV2.N54-sCD59-Fc4-CNP36 | |
| Vector GGP | AAV2.N54-sCD59----- C3i-Fc4-CNP36 | |
| | AAV2.N54-C3i-Fc4-endostatin (Vector | |
| | EAP) sham | |
| Vector GKA | AAV2.N54-C3i-Fc4-endostatin (Vector | |
| | EAP) | |
| Vector GKK | AAV2.N54-Aflibercept (Vector ACT)- | |
| | linker-C3i | |
| Vector CPE | AAV2.N54-Fc4-(G4S)4-CNP36 | |
| Vector GGQ | AAV2.N54-C3i- Fc4-CNP36 | |
| Vector GGE | AAV2.N54-(DK)C3i- Fc4-CNP36 | |
| Vector GKC | AAV2.N54-(DK) C3i- Fc4-endostatin | |
| Vector KKT | AAV2.N54-C3i(N15Q)-Fc4-CNP36 | |
| Vector KKA | AAV2.N54-C3i(N15Q)-Fc4-Endostatin | |
| Vector KMR, | AAV2.N54-C3i(N15Q)-Fc4-C3i514 | |
| Vector KPT | | |
| Vector KPC | AAV2.N54-C3i(N15Q)-Fc4 | |
| Vector KPP | AAV2.N54-C3i(N15Q)-Fc4-Endostatin | |

Tri-GOI

| | | |
|---|---|---|
| | AAV2.N54 -C3i-Fc4-CNP36-Furin-- | |
| | sCD59-sham | |
| Vector GKR | AAV2.N54 -sCD59-furin 2A-C3i-Fc4- | SEQ ID NO: 169-Vector |
| | CNP36 | GKR |
| Vector GMM | AAV2.N54 -sCD59-furin 2A- endostatin- | SEQ ID NO: 169-Vector |
| | linker-C3i | GMM |
| Vector GMR | AAV2.N54 -sCD59-furin 2A- Aflibercept- | SEQ ID NO: 169-Vector |
| | linker-C3i | GMR |
| | AAV2.N54-C3i-Fc4-CNP36-Furin-mCD59 | |
| | AAV2.N54-C3i-Fc4-endostatin (Vector | |
| | EAP)-Furin sCD59 sham | |
| | AAV2.N54-C3i-Fc4-endostatin (Vector | |
| | EAP)-Furin sCD59 | |

The expression level of the vector encoding the multiple angiogenesis inhibitors encoded can be measured. For example, enzyme-linked immunoassay (ELISA), Western blotting, or SDS-PAGE can quantify expression level of the angiogenesis inhibitors in cell lysate or in cell culture supernatants. After confirming expression of the multiple angiogenesis inhibitors, therapeutic efficacy of said multiple angiogenesis inhibitors can be determined in both cellular or animal model. For example, therapeutic efficacy of co-expressing of the multiple angiogenesis inhibitors from a vector can be assessed in an N-methyl-D-aspartate (NMDA) excitotoxicity mouse model of retinal degeneration; rat model of partial optic nerve transection (pONT); or neuroprotective effects and treatment efficacy glaucoma model.

### Example 2. rBV Amplification and AAV Production

### Cell Culture Maintenance

SF-RVF cells were cultured in ESF AF media (Expression Systems, Davis, CA) containing in Corning bottles with gentle shaking at 160 RPM and 28° C. Once cell density reached to ~1×10⁷ cells/mL, they were split at a ratio of 1:2 to 1:8 with fresh medium and continuously cultured for maintenance.

### Bacmid Transfection and rBV Amplification

Recombinant baculovirus expressing capsid protein (AAV2.N54 or AAV6.N54) and GOP were generated using a Bac-to-Bac baculovirus Expression System (Thermo Fischer Scientific, Fremont, CA) and GenJet DNA transfection reagent. The recombinant baculovirus was amplified by infecting 50-100 mL of SF-RVF cell culture at 1:200 (v/v) in 250-mL Corning bottles with shaking at 180 RPM and 28° C. for three days. The baculovirus titer was detected by qPCR. AAV production and purification

AAV was produced by co-infecting 200 mL of SF-RVF cell culture at 5×10⁶ cells/mL with the mixture of rBV-AAV2.N54 (AAV6.N54) and rBV-GOI at MOIs of 400-800 based on the rBV titers measured by QPCR analysis. The rBV infected culture was incubated with shaking at 180 RPM and 28° C. for three days. The cell pellets were harvested and lysed in lysis buffer (1% (w/v) Sarkosyl, 1% (v/v) Triton X-100, 10 mM Tris·HCl pH 8.0, 2 M Urea, 2 mM MgCl₂, and 25 IU/mL Benzonase™) at 37° C. with shaking at 300 RPM/min for 1 hour. At the end of the incubation, NaCl stock (5M) was added to the lysate to the final concentration of 0.5-1 M and the lysate was centrifuged at 8000 RPM for 20 min. Cleared lysate was purified by two rounds of CsCl gradient ultracentrifugation. The full AAV band was needle pulled and buffer exchanged into ACI Formulation Buffer 1a using a PD-10 desalting column. Sterile filtered AAV was quantified by ddPCR analysis using GOI specific primers/probes.

### Example 3. Cloning and Expression of C3i-Fc4-CNP36 Fusion Gene

C3i-Fc4-CNP36 fusion gene with human antibody heavy chain secretion signal peptide-encoding sequences at the 5′-end was constructed into a single-strand AAV vector, Vector ERP, and named with Vector GAM (FIG. 5A, FIG. 5B, or FIG. 5C). After the designed human antibody heavy chain secretion signal peptide-C31-3×GGGGS fusion proteins were reverse translated into DNA sequences using the Jcat program (Technische Universität Braunschweig, Lower Saxony, Germany) with Homo sapiens codon output, the partial fusion gene was further modified manually to adjust the GC content and sent for DNA synthesis in Integrated DNA Technologies, Inc. (Coralville, Iowa). While the synthetic DNA fragment was successfully cloned into Vector ETP, plasmid DNA was identified by restriction digestion, and sequences between two ITRs from plasmid DNA of the correct colonies were verified by compete sequencing Table 4.

### Transient Expression of the Vectors in Mammalian Cell Culture System

Human HEK293LTV cells were used for fusion gene expression in this study. They were cultured in DMEM medium (Thermo Fisher Scientific) with 10% FBS (ATCC, Manassas, VA) in a CO₂ incubator at 37° C. For maintenance passage, cells were split by 1:10 twice a week. For transfection, cells were seeded on 6-well plates (Corning, NY) at 0.75×10⁶ cells/well in 2 mL of medium overnight. One or two hours before transfection, the culture medium was replaced with DMEM medium with 2% FBS. For the cells on each well, 2 μg of plasmid DNA was diluted into 300 μL of 150 mM NaCl before 8 μL of PEImax (Polysciences, Germany) was added. After the mixture was incubated at room temperature for 20 min, it was added to the cells dropwise and incubated at 37° C. in the CO₂ incubator for 3 days. Then, medium was harvested for further experiments, and 3 mL of fresh medium was added into the cells. In the same, the medium was collected 3 days later again. Additionally, when the relatively large amount of proteins was needed, T125 flasks were used and the amount of plasmid DNA and reagents amount was increased accordingly.

While Vector GAM plasmid was prepared by Xtra Maxi Plus EF (Macherey-Nagel SAS, France), it was confirmed by restriction digestion using four different enzymes. In this study, the plasmid, Vector EKQ with EGFP gene was used as transfection control, and the plasmid, Vector CPE with the Fc4-CNP36 gene as protein expression control. After HEK293LTV cells were seeded onto 6-well plates 1 day prior to transient transfection, each transfection was performed with 2 μg/well DNA using PEImax. To evaluate the transfection efficiency, cells transfected with Vector EKQ was observed under fluorescence microscope (Data not shown). Cell culture supernatant at 3 days and 6 days post-transfection were collected and analyzed for protein expression.

### Detection and Quantification of C3i-Fc4-CNP36 Fusion Protein

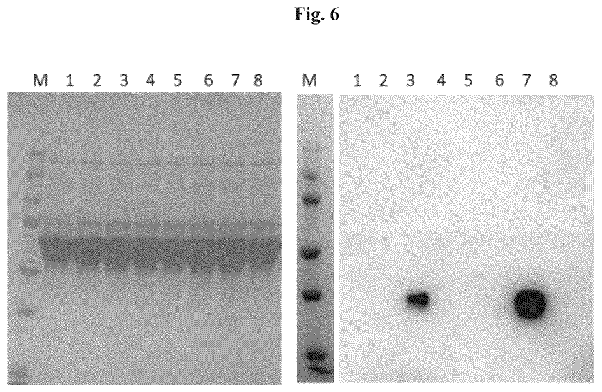

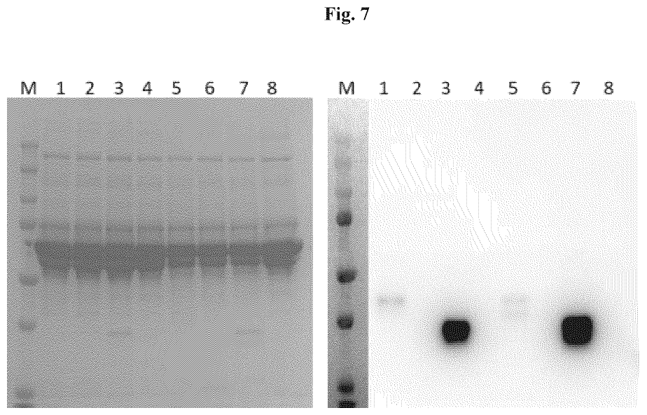

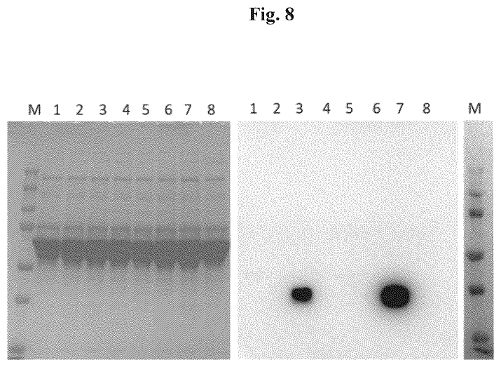

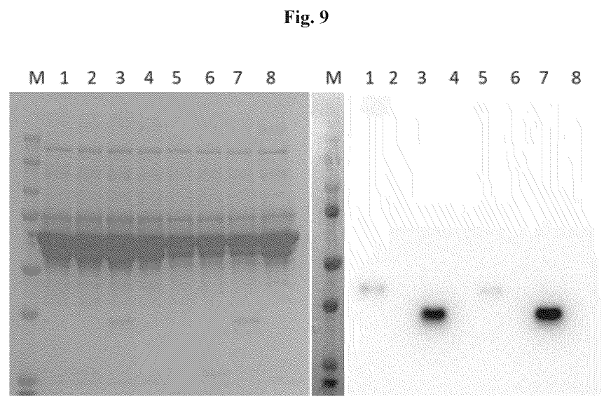

C3i-Fc4-CNP36 fusion proteins (Table 5) were detected by SDS-PAGE and Western blot analysis. HEK293LTV cell media (supernatants) was collected at 3 days and 6 days after plasmid DNA was transfected into the cells on 6-well plates or in the T125 flasks. A total volume of 26 μL of supernatants was mixed with 10 μl of 4× loading buffer and 4 μl of 10× reducing buffer and loaded onto the Novex™ WedgeWell™ Tris-Glycine gels (Thermo Fisher Scientific) for electrophoresis. After the proteins were run at 150 v for about 1.5 hour, one gel was stained by SimplyBlue™ SafeStain (Thermo Fisher Scientific), de-stained by water, and imaged using a digital camera (FIG. 6, FIG. 7, FIG. 8 and FIG. 9). On the other hand, another gel was subsequently transferred onto PVDF membranes using Trans-Blot Turbo Transfer System (Bio-Rad, Hercules, CA, USA). Immediately after membranes were treated with casein blocker in PBS (Thermo Scientific, Waltham, MA, USA) for 1 hour at room temperature, they were probed with HRP-conjugated goat anti-human IgG1 Fc antibody (A01854-200) (Genscript, Nanjing, China) or rat anti-human CNP antibodies (MAB31271) (Biotechne, Minneapolis, MN) followed by the use of the goat-HRP-conjugated goat-anti-rat IgG secondary antibody (1:10,000) (31470) (Thermo Fisher Scientific). Proteins were detected using the Supersignal™ West Atto Ultimate sensitivity kit (Thermo Fisher Scientific) and images were captured by Gel Doc™ XR+ with Image Lab™ Software (Bio-Rad, Hercules, CA) (FIG. 6, FIG. 7, FIG. 8, and FIG. 9). The SDS-PAGE and Western blot results show that the C3i-Fc4-CNP36 fusion protein in culture supernatant of the 3- or 6-days Vector GAM-transfected cells was detected, but not in the Vector EKQ or non-transfected cell culture supernatant. However, the expression level of C3i-Fc4-CNP36 fusion proteins for the Vector GAM plasmid is much lower than the Fc4-CNP36 fusion protein expressed by the plasmid, Vector CPE on the 6-well plates or in the T125 flasks using either HRP-conjugated goat anti-human IgG1 Fc antibody or rat anti-human CNP antibodies.

Also, the C3i-Fc4-CNP36 fusion proteins concentration in the supernatants was measured by sandwich ELISA. After 96-well microplates were coated with anti-human CNP antibodies (MAB31271) (Biotechne, Minneapolis, MN) and incubated at 4° C. overnight, they were treated with casein blocker in PBS at room temperature for 1 hr. Then CNP protein standards or supernatant samples were added into the microplates. After wash for three time, the Biotin-labelled goat-anti-human IgG Fc (ab98618) (Abcam, Waltham, MA) was applied to the plates followed by the use of HRP-Streptavidin conjugate (ab7403) (Abcam, Waltham, MA). The color reaction was developed using 1-Step™ Ultra TMB-ELISA Substrate Solution (ab171523) (Abcam, Waltham, MA) and stopped by the addition of 2M HCl. Finally, the microplate was read and recorded in the SpectaMax iD3 (Molecular Devices, San Jose, CA). The ELISA data was plotted and analyzed by GraphPad Prism 9.5.1 (Dotmatics, Boston, Massachusetts). Table 6 demonstrates that the C3i-Fc4-CNP36 fusion protein concentration in the 3d and 6d supernatant is 4.87 and 8.97 ug/mL, respectively. Similarly, when T125 flasks were used, the fusion protein concentration in the 3d and 6d supernatant is 29.02 and 16.09 μg/mL, respectively (Table 7). The fusion protein concentration measured by ELISA was much higher than the SDS-PAGE and Western blot.

The C3i-Fc4-CNP36 fusion protein in culture supernatant of the 3- or 6-days Vector GAM-transfected cells on the 6-well plates was detected by HRP-conjugated goat anti-human IgG1 Fc antibody, but not in the Vector EKQ or non-transfected cell culture supernatant. However, C3i-Fc4-CNP36 fusion protein expression level for the Vector GAM plasmid was much lower than the Fc4-CNP36 fusion protein expressed by the plasmid, Vector CPE. The resulting Western blot results are shown in FIG. 6. Lane content: 1. Vector GAM 3d; 2. Vector EKQ 3d; 3. Vector CME 3d; 4. Non-transfected 3d; 5. Vector GAM 6d; 6. Vector EKQ 6d; 7. Vector CME 6d; 8. Non-transfected 6d.

The C3i-Fc4-CNP36 fusion protein in culture supernatant of the 3- or 6-days Vector GAM-transfected cells in the T125 flasks was detected by HRP-conjugated goat anti-human IgG1 Fc antibody, but not in the Vector EKQ or non-transfected cell culture supernatant. However, C3i-Fc4-CNP36 fusion protein expression level for the Vector GAM plasmid showed much lower than the Fc4-CNP36 fusion protein expressed by the plasmid, Vector CPE. The resulting Western blot results are shown in FIG. 7. Lane content: 1. Vector GAM 3d; 2. Vector EKQ 3d; 3. Vector CME 3d; 4. non-transfected 3d; 5. Vector GAM 6d; 6. Vector EKQ 6d; 7. Vector CME 6d; 8. non-transfected 6d.

The C3i-Fc4-CNP36 fusion protein in culture supernatant of the 3- or 6-days Vector GAM-transfected cells on 6-well plates was detected by rat-anti-human CNP antibody, but not in the Vector EKQ or non-transfected cell culture supernatant. However, C3i-Fc4-CNP36 fusion protein expression level for the Vector GAM plasmid was much lower than the Fc4-CNP36 fusion protein expressed by the plasmid, Vector CPE. The resulting Western blot results are shown in FIG. 8. Lane content: 1. Vector GAM 3d; 2. Vector EKQ 3d; 3. Vector CME 3d; 4. Non-transfected 3d; 5. Vector GAM 6d; 6. Vector EKQ 6d; 7. Vector CME 6d; 8. Non-transfected 6d.

The C3i-Fc4-CNP36 fusion protein in culture supernatant of the 3- or 6-days Vector GAM-transfected cells in T125 flasks was detected by rat-anti-human CNP antibody, but not in the Vector EKQ or non-transfected cell culture supernatant. However, C3i-Fc4-CNP36 fusion protein expression level for the Vector GAM plasmid was much lower than the Fc4-CNP36 fusion protein expressed by the plasmid, Vector CPE. The resulting Western blot results are shown in FIG. 9. Lane content: 1. Vector GAM 3d; 2. Vector EKQ 3d; 3. Vector CME 3d; 4. non-transfected 3d; 5. Vector GAM 6d; 6. Vector EKQ 6d; 7. Vector CME 6d; 8

TABLE 4
DNA sequences of plasmids used in Example 3

| SEQ | | |
|---|---|---|
| ID | Clone | |
| NO | No. | Sequence |
| SEQ | Vector | CATTCGCCATTCAGGCTGCAAATAAGCGTTGATATTCAGTCAA |
| ID | GAM | TTACAAACATTAATAACGAAGAGATGACAGAAAAATTTTCAT |
| NO: | | TCTGTGACAGAGAAAAAGTAGCCGAAGATGACGGTTTGTCAC |
| 101 | | ATGGAGTTGGCAGGATGTTTGATTAAAAACATAACAGGAAGA |
| | | AAAATGCCCCGCTGTGGGCGGACAAAATAGTTGGGAACTGGG |
| | | AGGGGTGGAAATGGAGTTTTTAAGGATTATTTAGGGAAGAGT |
| | | GACAAAATAGATGGGAACTGGGTGTAGCGTCGTAAGCTAATA |
| | | CGAAAATTAAAAATGACAAAATAGTTTGGAACTAGATTTCAC |
| | | TTATCTGGTTCGGATCTCCTAGGGGTACCGGGCCCCCCCTCGA |
| | | GGTCGACGGTATCGATGGATCCGATATCGAATTCAAGCGCGC |
| | | TCGCTCGCTCACTGAGGCCGCCCGGGCAAAGCCCGGGCGTCG |
| | | GGCGACCTTTGGTCGCCCGGCCTCAGTGAGCGAGCGAGCGCG |
| | | CAGAGAGGGAGTGGCCAACTCCATCACTAGGGGTTCCTTGTA |
| | | GTTAATGATTAACCCGCCATGCTACTTATCTACGTACTCTGGA |
| | | GACGACTTACGGTAAATGGCCCGCCTGGCTGACCGCCCAACG |
| | | ACCCCCGCCCATTGACGTCAATAATGACGTATGTTCCCATAGT |
| | | AACGCCAATAGGGACTTTCCATTGACGTCAATGGGTGGAGTA |
| | | TTTACGGTAAACTGCCCACTTGGCAGTACATCAAGTGTATCAT |
| | | ATGCCAAGTCCGCCCCCTATTGACGTCAATGACGGTAAATGG |
| | | CCCGCCTGGCATTATGCCCAGTACATGACCTTACGGGACTTTC |
| | | CTACTTGGCAGTACATCTACGTATTAGTCATCGCTATTACCAT |
| | | GGTCGAGGTGAGCCCCACGTTCTGCTTCACTCTCCCCATCTCC |
| | | CCCCCCTCCCCACCCCCAATTTTGTATTTATTTATTTTTTAATT |
| | | ATTTTGTGCAGCGATGGGGGCGGGGGGGGGGGGGGGGCGCG |
| | | CGCCAGGCGGGGCGGGGCGGGGCGAGGGGCGGGGCGGGGCG |
| | | AGGCGGAGAGGTGCGGCGGCAGCCAATCAGAGCGGCGCGCT |
| | | CCGAAAGTTTCCTTTTATGGCGAGGCGGCGGCGGCGGCGGCC |
| | | CTATAAAAAGCGAAGCGCGCGGCGGGCGGGAGTCGCTGCGA |
| | | CGCTGCCTTCGCCCCGTGCCCCGCTCCGCCGCCGCCTCGCGCC |
| | | GCCCGCCCCGGCTCTGACTGACCGCGTTACTCCCACAGAGAT |
| | | CTCGTTTAGTGAACCGTCAGATCCTCACTCTCTTCCGCATCGC |
| | | TGTCTGCGAGGGCCAGCTGTTGGGCTCGCGGTTGAGGACAAA |
| | | CTCTTCGCGGTCTTTCCAGTACTCTTGGATCGGAAACCCGTCG |
| | | GCCTCCGAACGGTACTCCGCCACCGAGGGACCTGAGCGAGTC |
| | | CGCATCGACCGGATCGGAAAACCTCTCGAGAAAGGCGTCTAA |
| | | CCAGTCACAGTCGCAAGGTAGGCTGAGCACCGTGGCGGGCGG |
| | | CAGCGGGTGGCGGTCGGGGTTGTTTCTGGCGGAGGTGCTGCT |
| | | GATGATGTAATTAAAGTAGGCGGTCTTGAGACGGCGGATGGT |
| | | CGAGGTGAGGTGTGGCAGGCTTGAGATCCAGCTGTTGGGGTG |
| | | AGTACTCCCTCTCAAAAGCGGGCATTACTTCTGCGCTAAGATT |
| | | GTCAGTTTCCAAAAACGAGGAGGATTTGATATTCACCTGGCC |
| | | CGATCTGGCCATACACTTGAGTGACAATGACATCCACTTTGCC |
| | | TTTCTCTCCACAGGTGTCCACTCCCAGGTCCAAGTTTAAACGC |
| | | CGCCACCATGGAGTTCGGCCTGAGCTGGCTGTTCCTGGTGGCC |
| | | ATCCTTAAGGGCGTGCAGTGCGAGGTGCAGCTGGGCATCTGC |
| | | GTGTGGCAGGACTGGGGCGCCCACCGCTGCACCAACGGCGGA |
| | | GGCGGAAGCGGCGGAGGCGGATCTGGCGGAGGCGGAAGCGA |
| | | CAAGAGAGTGGAGAGCAAGTACGGCCCCCCCTGCCCCCCCTG |
| | | CCCCGCCCCCGAGTTCCTGGGCGGCCCCAGCGTGTTCCTGTTC |
| | | CCCCCCAAGCCCAAGGACACCCTGATGATCAGCAGAACCCCC |
| | | GAGGTGACCTGCGTGGTGGTGGACGTGAGCCAGGAGGACCCC |
| | | GAGGTGCAGTTCAACTGGTACGTGGACGGCGTGGAGGTGCAC |
| | | AACGCCAAGACCAAGCCCAGAGAGGAGCAGTTCAACAGCAC |
| | | CTACAGAGTGGTGAGCGTGCTGACCGTGCTGCACCAGGACTG |
| | | GCTGAACGGCAAGGAGTACAAGTGCAAGGTGAGCAACAAGG |
| | | GCCTGCCCAGCAGCATCGAGAAGACCATCAGCAAGGCCAAG |
| | | GGCCAGCCCAGAGAGCCCCAGGTGTACACCCTGCCCCCCAGC |
| | | CAGGAGGAGATGACCAAGAACCAGGTGAGCCTGACCTGCCTG |
| | | GTGAAGGGCTTCTACCCCAGCGACATCGCCGTGGAGTGGGAG |
| | | AGCAACGGCCAGCCCGAGAACAACTACAAGACCACCCCCCCC |
| | | GTGCTGGACAGCGACGGCAGCTTCTTCCTGTACAGCAGACTG |
| | | ACCGTGGACAAGAGCAGATGGCAGGAGGGCAACGTGTTCAG |
| | | CTGCAGCGTGATGCACGAGGCCCTGCACAACCACTACACCCA |
| | | GAAGAGCCTGAGCCTGAGCCTGGGCAAGGGCGGAGGCGGAA |
| | | GCGGCGGAGGCGGATCTGGCGGAGGCGGAAGCGGCGGAGGC |
| | | GGCTCTGAGCACCCCAACGCGCGCAAATACAAAGGAGCCAAC |
| | | AAGAAGGGCTTGTCCAAGGGCTGCTTCGGCCTCAAGCTGGAC |
| | | CGAATCGGCTCCATGAGCGGCCTGGGATGTTAACTCGAGAAT |
| | | CAACCTCTGGATTACAAAATTTGTGAAAGATTGACTGGTATTC |
| | | TTAACTATGTTGCTCCTTTTACGCTATGTGGATACGCTGCTTTA |
| | | ATGCCTTTGTATCATGCTATTGCTTCCCGTATGGCTTTCATTTT |
| | | CTCCTCCTTGTATAAATCCTGGTTGCTGTCTCTTTATGAGGAG |
| | | TTGTGGCCCGTTGTCAGGCAACGTGGCGTGGTGTGCACTGTGT |
| | | TTGCTGACGCAACCCCCACTGGTTGGGGCATTGCCACCACCTG |
| | | TCAGCTCCTTTCCGGGACTTTCGCTTTCCCCCTCCCTATTGCCA |
| | | CGGCGGAACTCATCGCCGCCTGCCTTGCCCGCTGCTGGACAG |
| | | GGGCTCGGCTGTTGGGCACTGACAATTCCGTGGTCTGTTCTCA |
| | | TCACATCATATCAAGGTTATATACCATCAATATTGCCACAGAT |
| | | GTTACTTAGCCTTTTAATATTTCTCTAATTTAGTGTATATGCAA |
| | | TGATAGTTCTCTGATTTCTGAGATTGAGTTTCTCATGTGTAAT |
| | | GATTATTTAGAGTTTCTCTTTCATCTGTTCAAATTTTTGTCTAG |
| | | TTTTATTTTTTACTGATTTGTAAGACTTCTTTTTATAATCTGCA |
| | | TATTACAATTCTCTTTACTGGGGTGTTGCAAATATTTTCTGTCA |
| | | TTCTATGGCCTGACTTTTCTTAATGGTTTTTTAATTTTAAAAAT |
| | | AAGTCTTAATATTCATGCAATCTAATTAACAATCTTTTCTTTGT |
| | | GGTTAGGACTTTGAGTCATAAGAAATTTTTCTCTACACTGAAG |
| | | TCATGATGGCATGCTTCTATATTATTTTCTAAAAGATTTAAAG |
| | | TTTTGCCTTCTCCATTTAGACTTATAATTCACTGGAATTTTTTT |
| | | GTGTGTATGGTATGACATATGGGTTCCCTTTTATTTTTTACATA |
| | | TAAATATATTTCCCTGTTTTTCTAAAAAAGAAAAAGATCATCA |
| | | TTTTCCCATTGTAAAATGCCATATTTTTTTCATAGGTCACTTAC |
| | | ATATATCAATGGGTCTGTTTCTGAGCTCTACTCTATTTTATCA |
| | | GCCTCACTGTCTATCCCCACACATCTCATGCTTTGCTCTAAAT |
| | | CTTGATATTTAGTGGAACATTCTTTCCCATTTTGTTCTACAAG |
| | | AATATTTTTGTTATTGTCTTTGGGCTTTCTATATACATTTTGAA |
| | | ATGAGGTTGACAAGTTACCTAGGAAAACTGTCTTCCTGCCCG |
| | | GGTGGCATCCCTGTGACCCCTCCCCAGTGCCTCTCCTGGCCCT |
| | | GGAAGTTGCCACTCCAGTGCCCACCAGCCTTGTCCTAATAAA |
| | | ATTAAGTTGCATCATTTTGTCTGACTAGGTGTCCTTCTATAAT |
| | | ATTATGGGGTGGAGGGGGGTGGTATGGAGCAAGGGGCCCAA |
| | | GTTGGGAAGAAACCTGTAGGGCCTGCGTACGTAGATAAGTAG |
| | | CATGGCGGGTTAATCATTAACTACAAGGAACCCCTAGTGATG |
| | | GAGTTGGCCACTCCCTCTCTGCGCGCTCGCTCGCTCACTGAGG |
| | | CCGGGCGACCAAAGGTCGCCCGACGCCCGGGCTTTGCCCGGG |
| | | CGGCCTCAGTGAGCGAGCGAGCGCGCAGCTGCTGGTACCGGG |
| | | CCCCCCCTCGAGGTCGACGGTATCGATGGATCCGATATCGAA |
| | | TTCAACCTGCAGGCATGCTCTGAGACAATAACCCTGATAAAT |
| | | GCTTCAATAATGTAGCCAACCACTAGAACTATAGCTAGAGTC |
| | | CTGGGCGAACAAACGATGCTCGCCTTCCAGAAAACCGAGGAT |
| | | GCGAACCACTTCATCCGGGGTCAGCACCACCGGCAAGCGCCG |
| | | CGACGGCCGAGGTCTTCCGATCTCCTGAAGCCAGGGCAGATC |
| | | CGTGCACAGCACCTTGCCGTAGAAGAACAGCAAGGCCGCCAA |
| | | TGCCTGACGATGCGTGGAGACCGAAACCTTGCGCTCGTTCGC |
| | | CAGCCAGGACAGAAATGCCTCGACTTCGCTGCTGCCCAAGGT |
| | | TGCCGGGTGACGCACACCGTGGAAACGGATGAAGGCACGAA |
| | | CCCAGTTGACATAAGCCTGTTCGGTTCGTAAACTGTAATGCAA |
| | | GTAGCGTATGCGCTCACGCAACTGGTCCAGAACCTTGACCGA |
| | | ACGCAGCGGTGGTAACGGCGCAGTGGCGGTTTTCATGGCTTG |
| | | TTATGACTGTTTTTTTGTACAGTCTATGCCTCGGGCATCCAAG |
| | | CAGCAAGCGCGTTACGCCGTGGGTCGATGTTTGATGTTATGG |
| | | AGCAGCAACGATGTTACGCAGCAGCAACGATGTTACGCAGCA |
| | | GGGCAGTCGCCCTAAAACAAAGTTAGGTGGCTCAAGTATGGG |
| | | CATCATTCGCACATGTAGGCTCGGCCCTGACCAAGTCAAATC |
| | | CATGCGGGCTGCTCTTGATCTTTTCGGTCGTGAGTTCGGAGAC |
| | | GTAGCCACCTACTCCCAACATCAGCCGGACTCCGATTACCTCG |
| | | GGAACTTGCTCCGTAGTAAGACATTCATCGCGCTTGCTGCCTT |
| | | CGACCAAGAAGCGGTTGTTGGCGCTCTCGCGGCTTACGTTCTG |
| | | CCCAGGTTTGAGCAGCCGCGTAGTGAGATCTATATCTATGATC |
| | | TCGCAGTCTCCGGCGAGCACCGGAGGCAGGGCATTGCCACCG |
| | | CGCTCATCAATCTCCTCAAGCATGAGGCCAACGCGCTTGGTG |
| | | CTTATGTGATCTACGTGCAAGCAGATTACGGTGACGATCCCG |
| | | CAGTGGCTCTCTATACAAAGTTGGGCATACGGGAAGAAGTGA |
| | | TGCACTTTGATATCGACCCAAGTACCGCCACCTAACAATTCGT |
| | | TCAAGCCGAGATCGGCTTCCCGGCCGCGGAGTTGTTCGGTAA |
| | | ATTGTCACAACGCCGCGAATATAGTCTTTACCATGCCCTTGGC |
| | | CACGCCCCTCTTTAATACGACGGGCAATTTGCACTTCAGAAA |
| | | ATGAAGAGTTTGCTTTAGCCATAACAAAAGTCCAGTATGCTTT |
| | | TTCACAGCATAACTGGACTGATTTCAGTTTACAACTATTCTGT |
| | | CTAGTTTAAGACTTTATTGTCATAGTTTAGATCTATTTTGTTCA |
| | | GTTTAAGACTTTATTGTCCGCCCACACCCGCTTACGCAGGGCA |
| | | TCCATTTATTACTCAACCGTAACCGATTTTGCCAGGTTACGCG |
| | | GCTGGTCTGCGGTGTGAAATACCGCACAGATGCGTAAGGAGA |
| | | AAATACCGCATCAGGCGCTCTTCCGCTTCCTCGCTCACTGACT |
| | | CGCTGCGCTCGGTCGTTCGGCTGCGGCGAGCGGTATCAGCTC |
| | | ACTCAAAGGCGGTAATACGGTTATCCACAGAATCAGGGGATA |
| | | ACGCAGGAAAGAACATGTGAGCAAAAGGCCAGCAAAAGGCC |
| | | AGGAACCGTAAAAAGGCCGCGTTGCTGGCGTTTTTCCATAGG |
| | | CTCCGCCCCCCTGACGAGCATCACAAAAATCGACGCTCAAGT |
| | | CAGAGGTGGCGAAACCCGACAGGACTATAAAGATACCAGGC |
| | | GTTTCCCCCTGGAAGCTCCCTCGTGCGCTCTCCTGTTCCGACC |
| | | CTGCCGCTTACCGGATACCTGTCCGCCTTTCTCCCTTCGGGAA |
| | | GCGTGGCGCTTTCTCAATGCTCACGCTGTAGGTATCTCAGTTC |
| | | GGTGTAGGTCGTTCGCTCCAAGCTGGGCTGTGTGCACGAACC |
| | | CCCCGTTCAGCCCGACCGCTGCGCCTTATCCGGTAACTATCGT |
| | | CTTGAGTCCAACCCGGTAAGACACGACTTATCGCCACTGGCA |
| | | GCAGCCACTGGTAACAGGATTAGCAGAGCGAGGTATGTAGGC |
| | | GGTGCTACAGAGTTCTTGAAGTGGTGGCCTAACTACGGCTAC |
| | | ACTAGAAGGACAGTATTTGGTATCTGCGCTCTGCTGAAGCCA |
| | | GTTACCTTCGGAAAAAGAGTTGGTAGCTCTTGATCCGGCAAA |
| | | CAAACCACCGCTGGTAGCGGTGGTTTTTTTGTTTGCAAGCAGC |
| | | AGATTACGCGCAGAAAAAAAGGATCTCAAGAAGATCCTTTGA |
| | | TCTTTTCTACGTGGTCTGACGCTCAGTGGAACGAAAACTCACG |
| | | TTAAGGGATTTTGGTCATGAGATTATCAAAAAGGATCTTCACC |
| | | TAGATCCTTTTAAATTAAAAATGAAGTTTTAAATCAATCTAAA |
| | | GTATATATGAGTAAACTTGGTCTGACGGTTACTCAGAAGAAC |
| | | TCGTCAAGAAGGCGATAGAAGGCGATGCGCTGCGAATCGGG |
| | | AGCGGCGATACCGTAAAGCACGAGGAAGCGGTCAGCCCATTC |
| | | GCCGCCAAGCTCTTCAGCAATATCACGGGTAGCCAACGCTAT |
| | | GTCCTGATAGCGGTCCGCCACACCCAGCCGGCCACAGTCGAT |
| | | GAATCCAGAAAAGCGGCCATTTTCCACCATGATATTCGGCAA |
| | | GCAGGCATCGCCATGGGTCACGACGAGATCCTCGCCGTCGGG |
| | | CATGCGCGCCTTGAGCCTGGCGAACAGTTCGGCTGGCGCGAG |
| | | CCCCTGATGCTCTTCGTCCAGATCATCCTGATCGACAAGACCG |
| | | GCTTCCATCCGAGTACGTGCTCGCTCGATGCGATGTTTCGCTT |
| | | GGTGGTCGAATGGGCAGGTAGCCGGATCAAGCGTATGCAGCC |
| | | GCCGCATTGCATCAGCCATGATGGATACTTTCTCGGCAGGAG |
| | | CAAGGTGAGATGACAGGAGATCCTGCCCCGGCACTTCGCCCA |
| | | ATAGCAGCCAGTCCCTTCCCGCTTCAGTGACAACGTCGAGCA |
| | | CAGCTGCGCAAGGAACGCCCGTCGTGGCCAGCCACGATAGCC |
| | | GCGCTGCCTCGTCCTGCAGTTCATTCAGGGCACCGGACAGGT |
| | | CGGTCTTGACAAAAAGAACCGGGCGCCCCTGCGCTGACAGCC |
| | | GGAACACGGCGGCATCAGAGCAGCCGATTGTCTGTTGTGCCC |
| | | AGTCATAGCCGAATAGCCTCTCCACCCAAGCGGCCGGAGAAC |
| | | CTGCGTGCAATCCATCTTGTTCAATCATACTCTTCCTTTTTCAA |
| | | TATTATTGAAGCATTTATCAGGGTTATTGTCTCATGAGCGGAT |
| | | ACATATTTGAATGTATTTAGAAAAATAAACAAATAGGGGTTC |
| | | CGCGCACATTTCCCCGAAAAGTGCCACCTGACGTCTAAGAAA |
| | | CCATTATTATCATGACATTAACCTATAAAAATAGGCGTATCAC |
| | | GAGGCCCTTTCGTCTCGCGCGTTTCGGTGATGACGGTGAAAA |
| | | CCTCTGACACATGCAGCTCCCGGAGACGGTCACAGCTTGTCT |
| | | GTAAGCGGATGCCGGGAGCAGACAAGCCCGTCAGGGCGCGT |
| | | CAGCGGGTGTTGGCGGGTGTCGGGGCTGGCTTAACTATGCGG |
| | | CATCAGAGCAGATTGTACTGAGAGTGCACCATATGCGGTGTG |
| | | AAATACCGCACAGATGCGTAAGGAGAAAATACCGCATCAGG |
| | | AAATTGTAAACGTTAATATTTTGTTAAAATTCGCGTTAAATTT |
| | | TTGTTAAATCAGCTCATTTTTTAACCAATAGGCCGAAATCGGC |
| | | AAAATCCCTTATAAATCAAAAGAATAGACCGAGATAGGGTTG |
| | | AGTGTTGTTCCAGTTTGGAACAAGAGTCCACTATTAAAGAAC |
| | | GTGGACTCCAACGTCAAAGGGCGAAAAACCGTCTATCAGGGC |
| | | GATGGCCCACTACGTGAACCATCACCCTAATCAAGTTTTTTGG |
| | | GGTCGAGGTGCCGTAAAGCACTAAATCGGAACCCTAAAGGGA |
| | | GCCCCCGATTTAGAGCTTGACGGGGAAAGCCGGCGAACGTGG |
| | | CGAGAAAGGAAGGGAAGAAAGCGAAAGGAGCGGGCGCTAG |
| | | GGCGCTGGCAAGTGTAGCGGTCACGCTGCGCGTAACCACCAC |
| | | ACCCGCCGCGCTTAATGCGCCGCTACAGGGCGCGTCC |
| SEQ | Vector | CATTCGCCATTCAGGCTGCAAATAAGCGTTGATATTCAGTCAA |
| ID | EKQ | TTACAAACATTAATAACGAAGAGATGACAGAAAAATTTTCAT |
| NO: | | TCTGTGACAGAGAAAAAGTAGCCGAAGATGACGGTTTGTCAC |
| 102 | | ATGGAGTTGGCAGGATGTTTGATTAAAAACATAACAGGAAGA |
| | | AAAATGCCCCGCTGTGGGCGGACAAAATAGTTGGGAACTGGG |
| | | AGGGGTGGAAATGGAGTTTTTAAGGATTATTTAGGGAAGAGT |
| | | GACAAAATAGATGGGAACTGGGTGTAGCGTCGTAAGCTAATA |
| | | CGAAAATTAAAAATGACAAAATAGTTTGGAACTAGATTTCAC |
| | | TTATCTGGTTCGGATCTCCTAGAGCTTACATTATTGAAGCATT |
| | | TATCAGGGTTATTGTCTCAGACCTGCAGGCAGCTGCGCGCTCG |
| | | CTCGCTCACTGAGGCCGCCCGGGCAAAGCCCGGGCGTCGGGC |
| | | GACCTTTGGTCGCCCGGCCTCAGTGAGCGAGCGAGCGCGCAG |
| | | AGAGGGAGTGGCCAACTCCATCACTAGGGGTTCCTGCGGCCG |
| | | CACGCGTACTAGTTATTAATAGTAATCAATTACGGGGTCATTA |
| | | GTTCATAGCCCATATATGGAGTTCCGCGTTACATAACTTACGG |
| | | TAAATGGCCCGCCTGGCTGACCGCCCAACGACCCCCGCCCAT |
| | | TGACGTCAATAATGACGTATGTTCCCATAGTAACGCCAATAG |
| | | GGACTTTCCATTGACGTCAATGGGTGGAGTATTTACGGTAAA |
| | | CTGCCCACTTGGCAGTACATCAAGTGTATCATATGCCAAGTAC |
| | | GCCCCCTATTGACGTCAATGACGGTAAATGGCCCGCCTGGCA |
| | | TTATGCCCAGTACATGACCTTATGGGACTTTCCTACTTGGCAG |
| | | TACATCTACGTATTAGTCATCGCTATTACCATGGTGATGCGGT |
| | | TTTGGCAGTACATCAATGGGCGTGGATAGCGGTTTGACTCAC |
| | | GGGGATTTCCAAGTCTCCACCCCATTGACGTCAATGGGAGTTT |
| | | GTTTTGGCACCAAAATCAACGGGACTTTCCAAAATGTCGTAA |
| | | CAACTCCGCCCCATTGACGCAAATGGGCGGTAGGCGTGTACG |
| | | GTGGGAGGTCTATATAAGCAGAGCTCGTTTAGTGAACCGTCA |
| | | GATCGCCTGGAGACGCCATCCACGCTGTTTTGACCTCCATAGA |
| | | AGACACCGGGACCGATCCAGCCTCCGTACCGGTTCGAACAGG |
| | | TAAGCGCCCCTAAAATCCCTTTGGGCACAATGTGTCCTGAGG |
| | | GGAGAGGCAGCGACCTGTAGATGGGACGGGGGCACTAACCC |
| | | TCAGGTTTGGGGCTTCTGAATGTGAGTATCGCCATGTAAGCCC |
| | | AGTATTTGGCCAATCTCAGAAAGCTCCTGGTCCCTGGAGGGA |
| | | TGGAGAGAGAAAAACAAACAGCTCCTGGAGCAGGGAGAGTG |
| | | CTGGCCTCTTGCTCTCCGGCTCCCTCTGTTGCCCTCTGGTTTCT |
| | | CCCCAGGTTCGAAGCGCGCAATTAACCCTCACTAAAGGGAAC |
| | | AAAAGCTGGAGCTCAATCGGACCGAAATTAATACGACTCACT |
| | | ATAGGGGAATTGTGAGCGGATAACAATTCCCCGGAGTTAATC |
| | | CGGGACCTTTAATTCAACCCAACACAATATATTATAGTTAAAT |
| | | AAGAATTATTATCAAATCATTTGTATATTAATTAAAATACTAT |
| | | ACTGTAAATTACATTTTATTTACAATCAAAGGAGATATACCAT |
| | | GGTGAGCAAGGGCGAGGAGCTGTTCACCGGGGTGGTGCCCAT |
| | | CCTGGTCGAGCTGGACGGCGACGTAAACGGCCACAAGTTCAG |
| | | CGTGTCCGGCGAGGGCGAGGGCGATGCCACCTACGGCAAGCT |
| | | GACCCTGAAGTTCATCTGCACCACCGGCAAGCTGCCCGTGCC |
| | | CTGGCCCACCCTCGTGACCACCCTGACCTACGGCGTGCAGTG |
| | | CTTCAGCCGCTACCCCGACCACATGAAGCAGCACGACTTCTTC |
| | | AAGTCCGCCATGCCCGAAGGCTACGTCCAGGAGCGCACCATC |
| | | TTCTTCAAGGACGACGGCAACTACAAGACCCGCGCCGAGGTG |
| | | AAGTTCGAGGGCGACACCCTGGTGAACCGCATCGAGCTGAAG |
| | | GGCATCGACTTCAAGGAGGACGGCAACATCCTGGGGCACAAG |
| | | CTGGAGTACAACTACAACAGCCACAACGTCTATATCATGGCC |
| | | GACAAGCAGAAGAACGGCATCAAGGTGAACTTCAAGATCCG |
| | | CCACAACATCGAGGACGGCAGCGTGCAGCTCGCCGACCACTA |
| | | CCAGCAGAACACCCCCATCGGCGACGGCCCCGTGCTGCTGCC |
| | | CGACAACCACTACCTGAGCACCCAGTCCGCCCTGAGCAAAGA |
| | | CCCCAACGAGAAGCGCGATCACATGGTCCTGCTGGAGTTCGT |
| | | GACCGCCGCCGGGATCACTCTCGGCATGGACGAGCTGTACAA |
| | | GTAAAGCGGCCGGCCGCACAGCTGTATACACGTGCAAGCCAG |
| | | CCAGAACTCGCCCCGGAAGACCCCGAGGATCTCGAGCACCAC |
| | | CATCACCATCACCATCACTAAGTGATTAACCTCAGGTGCAGG |
| | | CTGCCTATCAGAAGGTGGTGGCTGGTGTGGGTACCCAATTCG |
| | | CCCTATAGTGAGTCGTATTACGCGCGCAGCGGCCGACCATGG |
| | | CCCAACTTGTTTATTGCAGCTTATAATGGTTACAAATAAAGCA |
| | | ATAGCATCACAAATTTCACAAATAAAGCATTTTTTTCACTGCA |
| | | TTCTAGTTGTGGTTTGTCCAAACTCATCAATGTATCTTATCAT |
| | | GTCTGGATCTCCGGACACGTGCGGACCGAGCGGCCGCAGGAA |
| | | CCCCTAGTGATGGAGTTGGCCACTCCCTCTCTGCGCGCTCGCT |
| | | CGCTCACTGAGGCCGGGCGACCAAAGGTCGCCCGACGCCCGG |
| | | GCTTTGCCCGGGCGGCCTCAGTGAGCGAGCGAGCGCGCAGCT |
| | | GCCTGCAGGCATGCAAGCGTAGCCAACCACTAGAACTATAGC |
| | | TAGAGTCCTGGGCGAACAAACGATGCTCGCCTTCCAGAAAAC |
| | | CGAGGATGCGAACCACTTCATCCGGGGTCAGCACCACCGGCA |
| | | AGCGCCGCGACGGCCGAGGTCTTCCGATCTCCTGAAGCCAGG |
| | | GCAGATCCGTGCACAGCACCTTGCCGTAGAAGAACAGCAAGG |
| | | CCGCCAATGCCTGACGATGGATCCTGTCTCAAAATCTCTGATG |
| | | TTACATTGCACAAGATAAAAATATATCATCATGAACAATAAA |
| | | ACTGTCTGCTTACATAAACAGTAATACAAGGGGTGTTATGAG |
| | | CCATATTCAACGGGAAACGTCTTGCTCAAGGCCGCGATTAAA |
| | | TTCCAACATGGATGCTGATTTATATGGGTATAAATGGGCTCGC |
| | | GATAATGTCGGGCAATCAGGTGCGACAATCTATCGACTTTAT |
| | | GGGAAGCCCGATGCGCCAGAGTTGTTTCTGAAACATGGCAAA |
| | | GGTAGCGTTGCCAATGATGTTACAGATGAGATGGTCAGACTA |
| | | AACTGGCTGACGGAATTTATGCCTCTTCCGACCATCAAGCATT |
| | | TTATCCGTACTCCTGATGATGCATGGTTACTCACCACTGCGAT |
| | | CCCCGGGAAAACAGCATTCCAGGTATTAGAAGAATATCCTGA |
| | | TTCAGGTGAAAATATTGTTGATGCGCTGGCAGTGTTCCTGCGC |
| | | CGGTTGCATTCGATTCCTGTTTGTAATTGTCCTTTTAACAGCG |
| | | ATCGCGTATTTCGTCTCGCTCAGGCGCAATCACGAATGAATA |
| | | ACGGTTTGGTTGATGCGAGTGATTTTGATGACGAGCGTAATG |
| | | GCTGGCCTGTTGAACAAGTCTGGAAAGAAATGCATAAGCTTT |
| | | TGCCATTCTCACCGGATTCAGTCGTCACTCATGGTGATTTCTC |
| | | ACTTGATAACCTTATTTTTGACGAGGGGAAATTAATAGGTTGT |
| | | ATTGATGTTGGACGAGTCGGAATCGCAGACCGATACCAGGAT |
| | | CTTGCCATCCTATGGAACTGCCTCGGTGAGTTTTCTCCTTCATT |
| | | ACAGAAACGGCTTTTTCAAAAATATGGTATTGATAATCCTGAT |
| | | ATGAATAAATTGCAGTTTCATTTGATGCTCGATGAGTTTTTCT |
| | | AATCAGAATTGGTTAATTGGTTGTAACACTGGCAGAGCATTA |
| | | CGCTGACTTGACGGGACGGCGCCTAGGCTCATGACCAAAATC |
| | | CCTTAACGTGAGTTTTCGTTCCACTGAGCGTCAGACCCCGTAG |
| | | AAAAGATCAAAGGATCTTCTTGAGATCCTTTTTTTCTGCGCGT |
| | | AATCTGCTGCTTGCAAACAAAAAAACCACCGCTACCAGCGGT |
| | | GGTTTGTTTGCCGGATCAAGAGCTACCAACTCTTTTTCCGAAG |
| | | GTAACTGGCTTCAGCAGAGCGCAGATACCAAATACTGTTCTT |
| | | CTAGTGTAGCCGTAGTTAGGCCACCACTTCAAGAACTCTGTA |
| | | GCACCGCCTACATACCTCGCTCTGCTAATCCTGTTACCAGTGG |
| | | CTGCTGCCAGTGGCGATAAGTCGTGTCTTACCGGGTTGGACTC |
| | | AAGACGATAGTTACCGGATAAGGCGCAGCGGTCGGGCTGAAC |
| | | GGGGGGTTCGTGCACACAGCCCAGCTTGGAGCGAACGACCTA |
| | | CACCGAACTGAGATACCTACAGCGTGAGCTATGAGAAAGCGC |
| | | CACGCTTCCCGAAGGGAGAAAGGCGGACAGGTATCCGGTAA |
| | | GCGGCAGGGTCGGAACAGGAGAGCGCACGAGGGAGCTTCCA |
| | | GGGGGAAACGCCTGGTATCTTTATAGTCCTGTCGGGTTTCGCC |
| | | ACCTCTGACTTGAGCGTCGATTTTTGTGATGCTCGTCAGGGGG |
| | | GCGGAGCCTATGGAAAAACGCCAGCAACGCGGCCTTTTTACG |
| | | GTTCCTGGCCTTTTGCTGAATTCAAGGGCGAAAAACCGTCTAT |
| | | CAGGGCGATGGCCCACTACGTGAACCATCACCCTAATCAAGT |
| | | TTTTTGGGGTCGAGGTGCCGTAAAGCACTAAATCGGAACCCT |
| | | AAAGGGAGCCCCCGATTTAGAGCTTGACGGGGAAAGCCGGC |
| | | GAACGTGGCGGAAGGGAAGAAAGCGAAAGGAGCGGGCGCTA |
| | | GGGCGCTGGCAAGTGTAGCGGTCACGCTGCGCGTAACCACCA |
| | | CACCCGCCGCGCTTAATGCGCCGCTACAGGGCGCGTC |
| SEQ | Vector | CATTCGCCATTCAGGCTGCAAATAAGCGTTGATATTCAGTCAA |
| ID | CPE | TTACAAACATTAATAACGAAGAGATGACAGAAAAATTTTCAT |
| NO: | | TCTGTGACAGAGAAAAAGTAGCCGAAGATGACGGTTTGTCAC |
| 103 | | ATGGAGTTGGCAGGATGTTTGATTAAAAACATAACAGGAAGA |
| | | AAAATGCCCCGCTGTGGGCGGACAAAATAGTTGGGAACTGGG |
| | | AGGGGTGGAAATGGAGTTTTTAAGGATTATTTAGGGAAGAGT |
| | | GACAAAATAGATGGGAACTGGGTGTAGCGTCGTAAGCTAATA |
| | | CGAAAATTAAAAATGACAAAATAGTTTGGAACTAGATTTCAC |
| | | TTATCTGGTTCGGATCTCCTAGGCTCAAGCAGTGATCAGATCC |
| | | AGACATGATAAGATACATTGATGAGTTTGGACAAACCACAAC |
| | | TAGAATGCAGTGAAAAAAATGCTTTATTTGTGAAATTTGTGAT |
| | | GCTATTGCTTTATTTGTAACCATTATAAGCTGCAATAAACAAG |
| | | TTAACAACAACAATTGCATTCATTTTATGTTTCAGGTTCAGGG |
| | | GGAGGTGTGGGAGGTTTTTTAAAGCAAGTAAAACCTCTACAA |
| | | ATGTGGTATGGCTGATTATGATCCTCTAGTACTTCTCGACAAG |
| | | CTCGGATCCTGCGCGCTCGCTCGCTCACTGAGGCCGCCCGGG |
| | | CAAAGCCCGGGCGTCGGGCGACCTTTGGTCGCCCGGCCTCAG |
| | | TGAGCGAGCGAGCGCGCAGAGAGGGAGTGGGGTTGATCTCTC |
| | | CCCAGCATGCCACACAAAAAACCAACACACAGATGTAATGAA |
| | | AATAAAGATATTTTATTTTCGAAGGTACCACCACGGAATTGTC |
| | | AGTGCCCAACAGCCGAGCCCCTGTCCAGCAGCGGGCAAGGCA |
| | | GGCGGCGATGAGTTCCGCCGTGGCAATAGGGAGGGGGAAAG |
| | | CGAAAGTCCCGGAAAGGAGCTGACAGGTGGTGGCAATGCCCC |
| | | AACCAGTGGGGGTTGCGTCAGCAAACACAGTGCACACCACGC |
| | | CACGTTGCCTGACAACGGGCCACAACTCCTCATAAAGAGACA |
| | | GCAACCAGGATTTATACAAGGAGGAGAAAATGAAAGCCATA |
| | | CGGGAAGCAATAGCATGATACAAAGGCATTAAAGCAGCGTAT |
| | | CCACATAGCGTAAAAGGAGCAACATAGTTAAGAATACCAGTC |
| | | AATCTTTCACAAATTTTGTAATCCAGAGGTTGATTCTCGAGTT |
| | | AACATCCCAGGCCGCTCATGGAGCCGATTCGGTCCAGCTTGA |
| | | GGCCGAAGCAGCCCTTGGACAAGCCCTTCTTGTTGGCTCCTTT |
| | | GTATTTGCGCGCGTTGGGGTGCTCAGAGCCGCCTCCGCCGCTT |
| | | CCGCCTCCGCCAGATCCGCCTCCGCCGCTTCCGCCTCCGCCCT |
| | | TGCCCAGGCTCAGGCTCAGGCTCTTCTGGGTGTAGTGGTTGTG |
| | | CAGGGCCTCGTGCATCACGCTGCAGCTGAACACGTTGCCCTC |
| | | CTGCCATCTGCTCTTGTCCACGGTCAGTCTGCTGTACAGGAAG |
| | | AAGCTGCCGTCGCTGTCCAGCACGGGGGGGGTGGTCTTGTAG |
| | | TTGTTCTCGGGCTGGCCGTTGCTCTCCCACTCCACGGCGATGT |
| | | CGCTGGGGTAGAAGCCCTTCACCAGGCAGGTCAGGCTCACCT |
| | | GGTTCTTGGTCATCTCCTCCTGGCTGGGGGGCAGGGTGTACAC |
| | | CTGGGGCTCTCTGGGCTGGCCCTTGGCCTTGCTGATGGTCTTC |
| | | TCGATGCTGCTGGGCAGGCCCTTGTTGCTCACCTTGCACTTGT |
| | | ACTCCTTGCCGTTCAGCCAGTCCTGGTGCAGCACGGTCAGCAC |
| | | GCTCACCACTCTGTAGGTGCTGTTGAACTGCTCCTCTCTGGGC |
| | | TTGGTCTTGGCGTTGTGCACCTCCACGCCGTCCACGTACCAGT |
| | | TGAACTGCACCTCGGGGTCCTCCTGGCTCACGTCCACCACCAC |
| | | GCAGGTCACCTCGGGGGTTCTGCTGATCATCAGGGTGTCCTTG |
| | | GGCTTGGGGGGGAACAGGAACACGCTGGGGCCGCCCAGGAA |
| | | CTCGGGGGCGGGGCAGGGGGGGCAGGGGGGGCCGTACTTGC |
| | | TCTCCACTCTCTTGTCGCACTGCACGCCCTTAAGGATGGCCAC |
| | | CAGGAACAGCCAGCTCAGGCCGAACTCCATGGTGGCGGCAGG |
| | | CCTAGAAGTAAAGGCAACATCCACTGAGGAGCAGTTCTTTGA |
| | | TTTGCACCACCACCGGATCCGGGACCTGAAATAAAAGACAAA |
| | | AAGACTAAACTTACCTGTGGGAGTAACGCGGTCAGTCAGAGC |
| | | CGGGGCGGGCGGCGCGAGGCGGCGGCGGAGCGGGGCACGGG |
| | | GCGAAGGCAGCGTCGCAGCGACTCCCGCCCGCCGCGCGCTTC |
| | | GCTTTTTATAGGGCCGCCGCCGCCGCCGCCTCGCCATAAAAG |
| | | GAAACTTTCGGAGCGCGCCGCTCTGATTGGCTGCCGCCGCAC |
| | | CTCTCCGCCTCGCCCCGCCCCGCCCCTCGCCCCGCCCCGCCCC |
| | | GCCTGGCGCGCGCCCCCCCCCCCCCCCCGCCCCCATCGCTGCA |
| | | CAAAATAATTAAAAAATAAATAAATACAAAATTGGGGGTGG |
| | | GGAGGGGGGGGAGATGGGGAGAGTGAAGCAGAACGTGGGGC |
| | | TCACCTCGAGCCATGGTAATAGCGATGACTAATACGTAGATG |
| | | TACTGCCAAGTAGGAAAGTCCCATAAGGTCATGTACTGGGCA |
| | | TAATGCCAGGCGGGCCATTTACCGTCATTGACGTCAATAGGG |
| | | GGCGTACTTGGCATATGATACACTTGATGTACTGCCAAGTGG |
| | | GCAGTTTACCGTAAATACTCCACCCATTGACGTCAATGGAAA |
| | | GTCCCTATTGGCGTTACTATGGGAACATACGTCATTATTGACG |
| | | TCAATGGGCGGGGGTCGTTGGGCGGTCAGCCAGGCGGGCCAT |
| | | TTACCGTAAGTTATGTAACGGGTACCGAATTCAGATCAGCTTC |
| | | CTAGGAACCCCTAGTGATGGAGTTGGCCACTCCCTCTCTGCGC |
| | | GCTCGCTCGCTCACTGAGGCCGGGCGACCAAAGGTCGCCCGA |
| | | CGCCCGGGCTTTGCCCGGGCGGCCTCAGTGAGCGAGCGAGCG |
| | | CGCCAGGATCCGAGCTTGTCGAGAAGTACTAGAGGATCATCA |
| | | AGCTGTAGCCAACCACTAGAACTATAGCTAGAGTCCTGGGCG |
| | | AACAAACGATGCTCGCCTTCCAGAAAACCGAGGATGCGAACC |
| | | ACTTCATCCGGGGTCAGCACCACCGGCAAGCGCCGCGACGGC |
| | | CGAGGTCTTCCGATCTCCTGAAGCCAGGGCAGATCCGTGCAC |
| | | AGCACCTTGCCGTAGAAGAACAGCAAGGCCGCCAATGCCTGA |
| | | CGATGCGTGGAGACCGAAACCTTGCGCTCGTTCGCCAGCCAG |
| | | GACAGAAATGCCTCGACTTCGCTGCTGCCCAAGGTTGCCGGG |
| | | TGACGCACACCGTGGAAACGGATGAAGGCACGAACCCAGTTG |
| | | ACATAAGCCTGTTCGGTTCGTAAACTGTAATGCAAGTAGCGT |
| | | ATGCGCTCACGCAACTGGTCCAGAACCTTGACCGAACGCAGC |
| | | GGTGGTAACGGCGCAGTGGCGGTTTTCATGGCTTGTTATGACT |
| | | GTTTTTTTGTACAGTCTATGCCTCGGGCATCCAAGCAGCAAGC |
| | | GCGTTACGCCGTGGGTCGATGTTTGATGTTATGGAGCAGCAA |
| | | CGATGTTACGCAGCAGCAACGATGTTACGCAGCAGGGCAGTC |
| | | GCCCTAAAACAAAGTTAGGTGGCTCAAGTATGGGCATCATTC |
| | | GCACATGTAGGCTCGGCCCTGACCAAGTCAAATCCATGCGGG |
| | | CTGCTCTTGATCTTTTCGGTCGTGAGTTCGGAGACGTAGCCAC |
| | | CTACTCCCAACATCAGCCGGACTCCGATTACCTCGGGAACTTG |
| | | CTCCGTAGTAAGACATTCATCGCGCTTGCTGCCTTCGACCAAG |
| | | AAGCGGTTGTTGGCGCTCTCGCGGCTTACGTTCTGCCCAGGTT |
| | | TGAGCAGCCGCGTAGTGAGATCTATATCTATGATCTCGCAGTC |
| | | TCCGGCGAGCACCGGAGGCAGGGCATTGCCACCGCGCTCATC |
| | | AATCTCCTCAAGCATGAGGCCAACGCGCTTGGTGCTTATGTG |
| | | ATCTACGTGCAAGCAGATTACGGTGACGATCCCGCAGTGGCT |
| | | CTCTATACAAAGTTGGGCATACGGGAAGAAGTGATGCACTTT |
| | | GATATCGACCCAAGTACCGCCACCTAACAATTCGTTCAAGCC |
| | | GAGATCGGCTTCCCGGCCGCGGAGTTGTTCGGTAAATTGTCA |
| | | CAACGCCGCGAATATAGTCTTTACCATGCCCTTGGCCACGCCC |
| | | CTCTTTAATACGACGGGCAATTTGCACTTCAGAAAATGAAGA |
| | | GTTTGCTTTAGCCATAACAAAAGTCCAGTATGCTTTTTCACAG |
| | | CATAACTGGACTGATTTCAGTTTACAACTATTCTGTCTAGTTT |
| | | AAGACTTTATTGTCATAGTTTAGATCTATTTTGTTCAGTTTAA |
| | | GACTTTATTGTCCGCCCACACCCGCTTACGCAGGGCATCCATT |
| | | TATTACTCAACCGTAACCGATTTTGCCAGGTTACGCGGCTGGT |
| | | CTGCGGTGTGAAATACCGCACAGATGCGTAAGGAGAAAATAC |
| | | CGCATCAGGCGCTCTTCCGCTTCCTCGCTCACTGACTCGCTGC |
| | | GCTCGGTCGTTCGGCTGCGGCGAGCGGTATCAGCTCACTCAA |
| | | AGGCGGTAATACGGTTATCCACAGAATCAGGGGATAACGCAG |
| | | GAAAGAACATGTGAGCAAAAGGCCAGCAAAAGGCCAGGAAC |
| | | CGTAAAAAGGCCGCGTTGCTGGCGTTTTTCCATAGGCTCCGCC |
| | | CCCCTGACGAGCATCACAAAAATCGACGCTCAAGTCAGAGGT |
| | | GGCGAAACCCGACAGGACTATAAAGATACCAGGCGTTTCCCC |
| | | CTGGAAGCTCCCTCGTGCGCTCTCCTGTTCCGACCCTGCCGCT |
| | | TACCGGATACCTGTCCGCCTTTCTCCCTTCGGGAAGCGTGGCG |
| | | CTTTCTCAATGCTCACGCTGTAGGTATCTCAGTTCGGTGTAGG |
| | | TCGTTCGCTCCAAGCTGGGCTGTGTGCACGAACCCCCCGTTCA |
| | | GCCCGACCGCTGCGCCTTATCCGGTAACTATCGTCTTGAGTCC |
| | | AACCCGGTAAGACACGACTTATCGCCACTGGCAGCAGCCACT |
| | | GGTAACAGGATTAGCAGAGCGAGGTATGTAGGCGGTGCTACA |
| | | GAGTTCTTGAAGTGGTGGCCTAACTACGGCTACACTAGAAGG |
| | | ACAGTATTTGGTATCTGCGCTCTGCTGAAGCCAGTTACCTTCG |
| | | GAAAAAGAGTTGGTAGCTCTTGATCCGGCAAACAAACCACCG |
| | | CTGGTAGCGGTGGTTTTTTTGTTTGCAAGCAGCAGATTACGCG |
| | | CAGAAAAAAAGGATCTCAAGAAGATCCTTTGATCTTTTCTAC |
| | | GGGGTCTGACGCTCAGTGGAACGAAAACTCACGTTAAGGGAT |
| | | TTTGGTCATGAGATTATCAAAAAGGATCTTCACCTAGATCCTT |
| | | TTAAATTAAAAATGAAGTTTTAAATCAATCTAAAGTATATATG |
| | | AGTAAACTTGGTCTGACAGTTACCAATGCTTAATCAGTGAGG |
| | | CACCTATCTCAGCGATCTGTCTATTTCGTTCATCCATAGTTGC |
| | | CTGACTCCCCGTCGTGTAGATAACTACGATACGGGAGGGCTT |
| | | ACCATCTGGCCCCAGTGCTGCAATGATACCGCGAGACCCACG |
| | | CTCACCGGCTCCAGATTTATCAGCAATAAACCAGCCAGCCGG |
| | | AAGGGCCGAGCGCAGAAGTGGTCCTGCAACTTTATCCGCCTC |
| | | CATCCAGTCTATTAATTGTTGCCGGGAAGCTAGAGTAAGTAG |
| | | TTCGCCAGTTAATAGTTTGCGCAACGTTGTTGCCATTGCTACA |
| | | GGCATCGTGGTGTCACGCTCGTCGTTTGGTATGGCTTCATTCA |
| | | GCTCCGGTTCCCAACGATCAAGGCGAGTTACATGATCCCCCA |
| | | TGTTGTGCAAAAAAGCGGTTAGCTCCTTCGGTCCTCCGATCGT |
| | | TGTCAGAAGTAAGTTGGCCGCAGTGTTATCACTCATGGTTATG |
| | | GCAGCACTGCATAATTCTCTTACTGTCATGCCATCCGTAAGAT |
| | | GCTTTTCTGTGACTGGTGAGTACTCAACCAAGTCATTCTGAGA |
| | | ATAGTGTATGCGGCGACCGAGTTGCTCTTGCCCGGCGTCAAT |
| | | ACGGGATAATACCGCGCCACATAGCAGAACTTTAAAAGTGCT |
| | | CATCATTGGAAAACGTTCTTCGGGGCGAAAACTCTCAAGGAT |
| | | CTTACCGCTGTTGAGATCCAGTTCGATGTAACCCACTCGTGCA |
| | | CCCAACTGATCTTCAGCATCTTTTACTTTCACCAGCGTTTCTG |
| | | GGTGAGCAAAAACAGGAAGGCAAAATGCCGCAAAAAAGGGA |
| | | ATAAGGGCGACACGGAAATGTTGAATACTCATACTCTTCCTTT |
| | | TTCAATATTATTGAAGCATTTATCAGGGTTATTGTCTCATGAG |
| | | CGGATACATATTTGAATGTATTTAGAAAAATAAACAAATAGG |
| | | GGTTCCGCGCACATTTCCCCGAAAAGTGCCACCTGAAATTGT |
| | | AAACGTTAATATTTTGTTAAAATTCGCGTTAAATTTTTGTTAA |
| | | ATCAGCTCATTTTTTAACCAATAGGCCGAAATCGGCAAAATC |
| | | CCTTATAAATCAAAAGAATAGACCGAGATAGGGTTGAGTGTT |
| | | GTTCCAGTTTGGAACAAGAGTCCACTATTAAAGAACGTGGAC |
| | | TCCAACGTCAAAGGGCGAAAAACCGTCTATCAGGGCGATGGC |
| | | CCACTACGTGAACCATCACCCTAATCAAGTTTTTTGGGGTCGA |
| | | GGTGCCGTAAAGCACTAAATCGGAACCCTAAAGGGAGCCCCC |
| | | GATTTAGAGCTTGACGGGGAAAGCCGGCGAACGTGGCGAGA |
| | | AAGGAAGGGAAGAAAGCGAAAGGAGCGGGCGCTAGGGCGCT |
| | | GGCAAGTGTAGCGGTCACGCTGCGCGTAACCACCACACCCGC |
| | | CGCGCTTAATGCGCCGCTACAGGGCGCGTC |

TABLE 5
Peptides and/or fusion protein sequences

| SEQ | | | |
|---|---|---|---|
| ID | Clone | | |
| NO: | ID | Protein | Protein sequences |
| 111 | Vector | C3i- | EVQLGICVWQDWGAHRCTNGGGGSGGGGSGGGGSDK |
| | GAM | Fc4- | RVESKYGPPCPPCPAPEFLGGPSVFLFPPKPKDTLMISRT |
| | | CNP36 | PEVTCVVVDVSQEDPEVQFNWYVDGVEVHNAKTKPRE |
| | | (CFC36) | EQFNSTYRVVSVLTVLHQDWLNGKEYKCKVSNKGLPS |
| | | | SIEKTISKAKGQPREPQVYTLPPSQEEMTKNQVSLTCLV |
| | | | KGFYPSDIAVEWESNGQPENNYKTTPPVLDSDGSFFLYS |
| | | | RLTVDKSRWQEGNVFSCSVMHEALHNHYTQKSLSLSL |
| | | | GKGGGGSGGGGSGGGGSGGGGSEHPNARKYKGANKK |
| | | | GLSKGCFGLKLDRIGSMSGLGC |
| 112 | Vector | EGFP | MVSKGEELFTGVVPILVELDGDVNGHKFSVSGEGEGDA |
| | EKQ | | TYGKLTLKFICTTGKLPVPWPTLVTTLTYGVQCFSRYPD |
| | | | HMKQHDFFKSAMPEGYVQERTIFFKDDGNYKTRAEVK |
| | | | FEGDTLVNRIELKGIDFKEDGNILGHKLEYNYNSHNVYI |
| | | | MADKQKNGIKVNFKIRHNIEDGSVQLADHYQQNTPIGD |
| | | | GPVLLPDNHYLSTQSALSKDPNEKRDHMVLLEFVTAAG |
| | | | ITLGMDELYK |
| 113 | Vector | Fc4- | DKRVESKYGPPCPPCPAPEFLGGPSVFLFPPKPKDTLMIS |
| | CPE | (G4S)4- | RTPEVTCVVVDVSQEDPEVQFNWYVDGVEVHNAKTKP |
| | | CNP36 | REEQFNSTYRVVSVLTVLHQDWLNGKEYKCKVSNKGL |
| | | | PSSIEKTISKAKGQPREPQVYTLPPSQEEMTKNQVSLTCL |
| | | | VKGFYPSDIAVEWESNGQPENNYKTTPPVLDSDGSFFLY |
| | | | SRLTVDKSRWQEGNVFSCSVMHEALHNHYTQKSLSLSL |
| | | | GKGGGGSGGGGSGGGGSGGGGSEHPNARKYKGANKK |
| | | | GLSKGCFGLKLDRIGSMSGLGC |

TABLE 6
The fusion protein concentration on 6-well plates
of HEK293 cells measured by the sandwich ELISA

| Vector ID. | |
|---|---|
| Vector GAM | Vector CME |

Days post transfection

| | 3 | 6 | 3 | 6 |
|---|---|---|---|---|
| CFC36 (μg/mL) | 4.87 | 8.97 | 7.02 | 16.14 |
| StDev (μg/mL) | 0.01 | 0.13 | 0.42 | 2.28 |
| Ratio | | | 1.44 | 1.80 |

TABLE 7
The fusion protein concentration on T125 flasks
of HEK293 cells measured by the sandwich ELISA

| Vector ID. | |
|---|---|
| Vector GAM | Vector CME |

Days post transfection

| | 3 | 6 | 3 | 6 |
|---|---|---|---|---|
| CFC36 (μg/mL) | 29.02 | 16.09 | 45.73 | 43.44 |
| StDev (μg/mL) | 1.26 | 0.18 | 0.40 | 1.92 |
| Ratio | | | 1.58 | 2.70 |

### Example 4. Cloning and Expression of Membrane-Bound and Soluble CD59 Genes

Designing and Molecular Cloning of Membrane-Bound and Soluble CD59 (sCD59) Genes

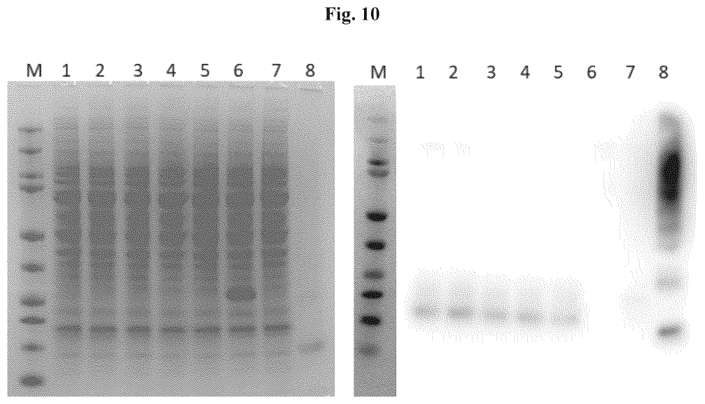

Membrane-bound CD59 (mCD59) with native signal peptide-encoding sequences at the 5′-end was constructed into a self-complementary AAV vector, Vector CPE. After mCD59 proteins were reverse translated into DNA sequences with Homo sapiens codon output using 4 different programs from Snapgene, Jcat, Geneart, and Genscript, the full-length mCD59 genes with different codon optimization were further modified manually to adjust the GC content and sent for DNA synthesis in Twist Bioscience (South San Francisco, CA). After the synthetic DNA fragment was successfully cloned into Vector CPE, all plasmid DNA was identified by restriction digestion and named with Vector GTM for wild-type mCD59, Vector GTP for the Snapgene-optimized mCD59, Vector GTQ for the Jcat-optimized, Vector GTR for the Geneart-optimized, Vector GAT for the Genscript-optimized (FIG. 10), and the sequences from all these plasmids between two ITRs were verified by compete sequencing Table 8. Additional information regarding sequence information is shown in Table 9.

Since wild-type and Snapgene-optimized mCD59 showed higher expression level among these genes, they were chosen for soluble CD59 expression. After the GPI-anchor peptide (GGTSLSEKTVLLLVTPFLAAAWSLHP)-encoding sequences were removed from the full-length CD59 gene, the wild-type and Snapgene-optimized sCD59 with native or human antibody heavy chain secretion signal peptide-encoding sequences at 5′ end was subcloned into a single-strand AAV vector, Vector ETP. Four different plasmids were obtained, for example, Vector GCK contains native secretion signal peptide-encoding sequences and sCD59 sequences; Vector GCM, human antibody heavy chain secretion signal peptide-encoding sequences and wild-type sCD59 sequences; Vector GEM, native secretion signal peptide-encoding sequences and Snapgene-optimized sCD59 gene; Vector GEP, human antibody heavy chain secretion signal peptide-encoding sequences and Snapgene-optimized sCD59 gene. In the same, all these plasmids were identified by restriction digestion, and the sequences from all these plasmids between two ITRs were verified by compete sequencing (Table 8).

Transient Expression of mCD59 Genes in Mammalian Cell Culture System

Human EXPI293 suspension cells were used for the mCD59 expression. Cells were cultured in BalanCD medium (Fujifilm Irvine Scientific, Santa Ana, CA) with 5 mM glutaMax™ (Thermo Fisher Scientific) in an orbit shaker at 37° C. Immediately prior to transfection using Mirus transfection reagent (San Francisco, CA), cells were diluted in a shake flask (Corning, NY) at 2.0×10⁶ cells/mL in 25 mL of medium. For each shake flask, 50 μg of plasmid DNA was diluted into 2.5 ml of VirusGEN® SELCT AAV Complex Formation Solution and Enhancer in a sterile tube before 75 μl of TransIT-VirusGEN® SELECT Reagent was added to the diluted DNA. After the mixture was incubated at room temperature for 15-30 minutes without additional agitation to allow transfection complexes to form, it was added to the cells dropwise and incubated at 37° C. in an orbit shaker for 2 days. Then the cells were harvested for further experiments. After the cells were washed with 1×PBS once, 1 mL of RIPA buffer (89901) (Thermo Fisher Scientific) with 1× protease inhibitor cocktail (1183670001) (Millipore Sigma, Burlington, MA) was added into 10×10⁶ cells. After the cells were sonicated for 10 seconds three times with 10 second intervals, samples were centrifuged at 12000×g for 15 min. The cell lysate was collected and the protein concentration was measured by Pierce™ BCA protein assay kit (23225) (Thermo Fisher Scientific).

Plasmids Vector GTM, Vector GTP, Vector GTQ, Vector GTR, and Vector GAT, were prepared by Xtra Maxi Plus EF (Macherey-Nagel SAS, France) and confirmed by restriction digestion. Vector EKQ containing EGFP gene was used as the transfection control. After EXPI293 suspension cells were diluted to 2.0×10⁶ cells/mL in 25 mL of medium, each transfection was performed with 50 μg of DNA using Mirus transfection reagent. To evaluate the transfection efficiency, cells transfected with Vector EKQ was observed under fluorescence microscope. The cell lysate was prepared, collected and analyzed for protein expression.

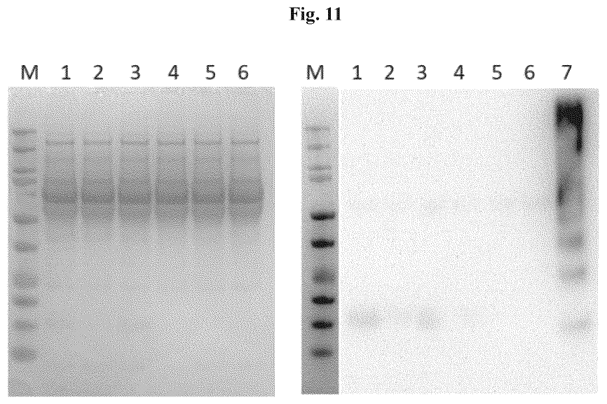

mCD59 proteins of the cell lysate were detected by SDS-PAGE and Western blot analysis. EXPI293 cells were collected at day 2 after the plasmid DNA was transfected into the cells. A total volume of 26 μL of cell lysate was mixed with 10 μl of 4× loading buffer and 4 μl of 10× reducing buffer and loaded onto the NuPAGE™ 4-12% Bis-Tris gels (Thermo Fisher Scientific) for electrophoresis. The proteins were run at 150 v for about 1 hour, one gel was stained by SimplyBlue™ SafeStain (Thermo Fisher Scientific), de-stained by water, and imaged using a digital camera (FIG. 11). Another gel was subsequently transferred onto PVDF membranes using Trans-Blot Turbo Transfer System (Bio-Rad, Hercules, CA, USA). Immediately after membranes were treated with casein blocker in PBS (Thermo Scientific, Waltham, MA, USA) for 1 hour at room temperature, they were probed with HRP-conjugated mouse-anti-human CD59 monoclonal antibody (sc-133170) (Santa Cruz Biotechnology, Dallas, TX). Proteins were detected using the Supersignal™ West Atto Ultimate sensitivity kit (Thermo Fisher Scientific) and images were captured by Gel Doc™ XR+ with Image Lab™ Software (Bio-Rad, Hercules, CA). The SDS-PAGE and Western blot results showed that the mCD59 was expressed in Vector GTM, Vector GTP, Vector GTQ, Vector GTR, and Vector GAT-transfected cells, but not in the Vector EKQ or non-transfected cells (FIG. 11). Moreover, Snapgene-optimized mCD59 has the highest expression level followed by wild-type mCD59, the Genscript-optimized, the Jcat-optimized, and the Geneart-optimized mCD59 shows the lowest expression level.

Transient Expression of sCD59 Genes in Mammalian Cell Culture System

Human HEK293LTV cells were used for sCD59 gene expression and were cultured in DMEM medium (Thermo Fisher Scientific) with 10% FBS (ATCC, Manassas, VA) in a CO₂ incubator at 37° C. For maintenance passage, cells were split by 1:10 twice a week. For transfection, cells were seeded on 6-well plates (Corning, NY) at 0.6×10⁶ cells/well in 2 mL media overnight. One or two hours before transfection, the culture medium was replaced with DMEM medium with 2% FBS. For each well, 2 μg of plasmid DNA was diluted into 300 μL of 150 mM NaCl before 8 μL of PEImax (Polysciences, Germany) was added. After the mixture was incubated at room temperature for 20 minutes, it was added to the cells dropwise and incubated at 37° C. in the CO₂ incubator. Three days later, all medium from each well was harvested for further experiments, and 3 mL of fresh medium was added to the cells. In the same, the medium was collected in 3 days one more time.

While the plasmids, Vector GCK, Vector GCM, Vector GEM and Vector GEP, were prepared by Xtra Maxi Plus EF (Macherey-Nagel SAS, France), it was confirmed by restriction digestion. In this study, the plasmid, Vector EKQ with EGFP gene was used as the transfection control. After HEK293LTV cells were seeded onto 6-well plates 1 day prior to transient transfection, each transfection was performed with 2 μg/well DNA using PEImax. To evaluate the transfection efficiency, cells transfected with Vector EKQ was observed under fluorescence microscope. Cell culture supernatants at 3 days and 6 days post-transfection were collected and analyzed for protein expression.

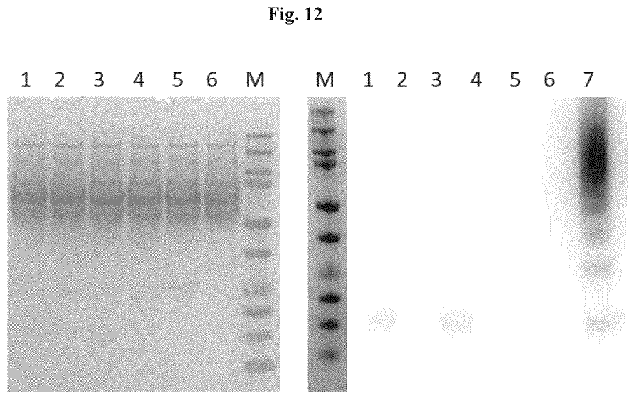

sCD59 proteins were detected by SDS-PAGE and Western blot analysis. Cell culture supernatant was collected at day 3 and 6 after plasmid DNA was transfected into the cells. A total volume of 26 μL of each supernatant was mixed with 10 μl of 4× loading buffer and 4 μl of 10× reducing buffer and loaded onto the NuPAGE™ 4-12% Bis-Tris gels (Thermo Fisher Scientific) for electrophoresis. After the proteins were run at 150 v for about 1 hour, one gel was stained by SimplyBlue™ SafeStain (Thermo Fisher Scientific), de-stained by water, and imaged using a digital camera (FIG. 11 and FIG. 12). On the other hand, another gel was subsequently transferred onto PVDF membranes using Trans-Blot Turbo Transfer System (Bio-Rad, Hercules, CA, USA). Immediately after membranes were treated with casein blocker in PBS (Thermo Scientific, Waltham, MA, USA) for 1 hour at room temperature, they were probed with HRP-conjugated mouse-anti-human CD59 monoclonal antibody (sc-133170) (Santa Cruz Biotechnology, Dallas, TX). Proteins were detected using the Supersignal™ West Atto Ultimate sensitivity kit (Thermo Fisher Scientific) and images were captured by Gel Doc™ XR+ with Image Lab™ Software (Bio-Rad, Hercules, CA). Western blot results show that the sCD59 was expressed in Vector GCK, 426, 436, and 437-transfected cells, but not in the Vector EKQ or non-transfected cells (FIG. 11 and FIG. 12). Moreover, sCD59 gene from the plasmids, Vector GCK and Vector GEM demonstrate much higher expression level than that from Vector GCM and Vector GEP.

The membrane-bound CD59 (mCD59) from the cell lysate was detected by HRP-conjugated anti-human CD59 antibody, but not in the Vector EKQ or non-transfected cells. The resulting Western blot results are shown in FIG. 10. Lane content: 1. Vector GTM; 2. Vector GTP; 3. Vector GTQ; 4. Vector GTR; 5. Vector GAT; 6. Vector EKQ; 7. Non-transfected; 8. Purified CD59 protein.

Soluble CD59 (sCD59) protein in the cell culture supernatant at day 3 was detected using HRP-conjugated anti-human CD59 antibody. The soluble CD59 (sCD59) from the cell culture supernatant at day 3 was detected by HRP-conjugated anti-human CD59 antibody, but not in the Vector EKQ or non-transfected cells. The resulting Western blot results are shown in FIG. 11. Lane content: 1. Vector GCK; 2. Vector GCM; 3. Vector GEM; 4. Vector GEP; 5. Vector EKQ; 6. Non-transfected; 7. Purified CD59 protein.

The soluble CD59 (sCD59) from the cell culture supernatant at day 6 was detected by HRP-conjugated anti-human CD59 antibody, but not in the Vector EKQ or non-transfected cells. The resulting Western blot images are shown in FIG. 12. Lane content: 1. Vector GCK; 2. Vector GCM; 3. Vector GEM; 4. Vector GEP; 5. Vector EKQ; 6. Non-transfected; 7. Purified CD59 protein.

sCD59 Protein Purification

A large-scale cell culture was prepared for Vector GEM protein purification. sCD59 protein was purified using AKTA Explorer 100 in four steps. First, sample clarification was performed to remove cellular debris by centrifugation followed by 0.2 μm filtration (Nalgene, Rochester, NY). The clarified harvest was directly transferred into downstream purification. Second, gel filtration column chromatography was performed using Sephadex G-25 (Cytiva, Marlborugh, MA) to remove cell culture medium from the cell culture supernatant containing the CD59 protein. After the G-25 desalting column was equilibrated with Tris buffer (20 mM Tris pH7.3, 100 mM NaCl, 0.005% F-68), the cell culture supernatant containing the sCD59 protein was loaded. The sCD59 protein was collected when the UV A280 increases to 5 mAU and collection was stopped when the UV A280 reduced to 5 mAU. Third, a strong anion exchange column chromatography using HQ resin (ThermoFisher, Walthan, MA) was performed on the G-25 desalted pool. The G-25 desalted product was diluted with XQ column equilibration buffer (20 mM Tris pH7.3, 0.005% F-68) to a conductivity <3 mS/cm and loaded onto an equilibrated HQ column with equilibration buffer (20 mM Tris pH7.3, 10 mM NaCl, 0.005% F-68). After loading and washing with equilibration buffer, the column was eluted using a 10-500 mM NaCl gradient. The elution peak was collected when the A280 increased to 5 mAU and the collection was ended when the UV A280 reduced to 5 mAU. Fourth, size exclusion column (SEC) chromatography using Superdex 75 (Cytiva, Marlborugh, MA) was performed to further remove the impurity from the collection of HQ purification. After the SEC column was equilibrated with PBS buffer, the HQ column chromatography collection was loaded. The CD59 protein elution peak was collected when the A280 increased to 5 mAU and the collection was ended when the UV A280 reduced to 5 mAU.

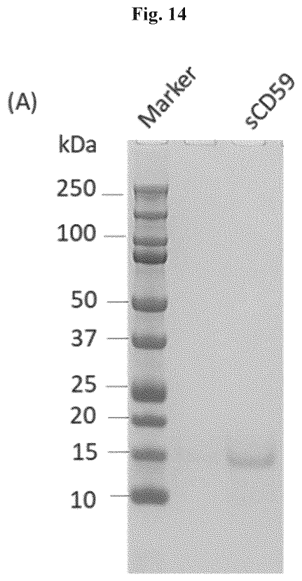

The purified CD59 sample was resolved by SDS-PAGE using 4-12% Bis-Tris gradient gels (Invitrogen™ Novex) according to the manufacturer's instructions. Protein bands were visualized by staining with Coomassie Blue. In the gel, the protein size is estimated to be around 14 kDa that is about 5 kDa larger than the theoretic molecular weight (FIG. 14). The result show that the sCD59 was of high purity. The size increase could have been due to the protein glycosylation. Lane content: 1. Vector GEM.

sCD59 N-Terminal Sequencing

The purified sCD59 proteins were sent for N-terminal sequencing for protein identification. After the sCD59 proteins were transferred onto the PVDF membrane and stained with SimplyBlue SafeStain (Thermo Fisher Scientific, Cat. #465034), a single band with approximately 8 ug proteins was excised and kept in the 5 mL of Eppendorf tube (data not shown). The bands were thoroughly de-stained by 50%, 60%, and 70% of methanol for 15 min sequentially. Then the blots were washed by milliQ water for 15 min. Airdry the blot overnight. The de-stained membrane was transferred to a fresh tube and stored at 4° C. until the sample was shipped on ice pack to Creative Proteomics (Shirley, NY, USA) for N-terminal first 5 amino acids identification. The protein N-terminal sequencing results showed that the first 4 out of 5 amino acids are identical to the predicted (Table 42). The results show that the sCD59 protein was correctly translated and processed for secretion.

TABLE 42
Vector GEM protein N-terminal sequencing results

| Cycle | Residue | Amino Acid |
|---|---|---|
| 1 | Blank | — |
| 2 | Standard | — |
| 3 | 1 | L |
| 4 | 2 | Q |
| 5 | 3 | — |
| 6 | 4 | Y |
| 7 | 5 | N |

sCD59 Function Assay

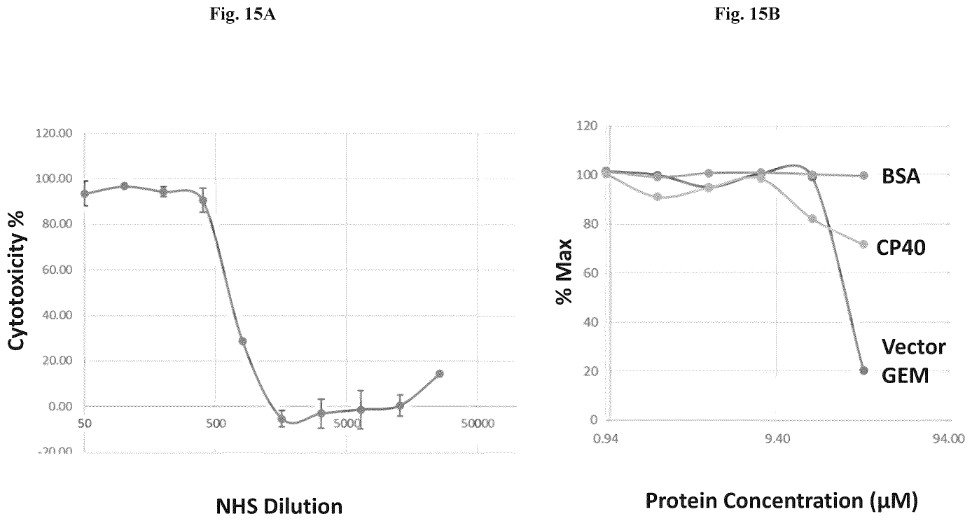

To test biological function of sCD59, cell lysis inhibition assay was performed using Hep1c1c7 cells. The serum dose causing approximately 70% cell lysis was determined for the new normal human serum (NHS) before the purified sCD59 proteins were used for cell lysis inhibition assay. Three hours prior to the experiment, 1×10⁴ cells per well were seeded onto a 96-well plate. Various doses of NHS were mixed with GVB2++ buffer (with Ca²⁺ and Mg²⁺) (Complement Technology, Cat. B100). 100 μL of mixture was added to each well. Background lysis was measured using GVB2++ buffer only and maximum lysis was measured by lysing all the cells with Maximum Lysis Buffer (GVB2++ plus 1% SDS). After cells were incubated with mixtures for 1 hour, 10 μL of WST-8 was added into each well of the plate. Again, the cells were incubated with WST-8 for 1-4 hours. The absorbance at 450 nm was measured using a microplate reader. To calculate the percentage of live cells, the absorbance was subtracted by the Max Lysis, divided by Background Lysis minus Max Lysis and multiplied by 100, which equaled the % of live cells. The % of dead cells was calculated by subtracting 100 by the % of live cells. In the GraphPad Prism, the % of cell death was plotted against dose of NHS, and the dose of NHS causing 50% and 70% hemolysis was respectively determined (FIG. 15A).

Using the NHS dose causing 70% cell lysis, the purified sCD59 proteins were used for cell lysis inhibition assay. The sCD59 proteins were 2× serially diluted and mixed with GVB2++ buffer in the presence of NHS, resulting in the final concentration of 30.11, 15.06, 7.53, 3.76, 1.88, and 0.94 μM. The benchmark, CP40 peptide, was also used in this study. The mixture for each sample was added into 4 wells. After quadruple readings was subtracted from the Max Lysis and averaged out, it was divided by 70% of cell lysis minus Max Lysis and multiplied by 100. The % of cell lysis was considered as % of cell lysis inhibition. Using GraphPad Prism software, the percentage of cell lysis inhibition was plotted against the molecular concentration of sCD59 proteins and IC50 value was estimated to be around 20 μM based on the data plotting (FIG. 15B). The results demonstrated that purified sCD59 could dramatically inhibit cell lysis at increasing concentration, and CP40 also showed the marginal inhibition, but the BSA control did not have an inhibitory effect at all.

TABLE 8
DNA sequences of plasmids used in Example 4

| SEQ ID NO | Clone no. | Sequence |
|---|---|---|
| 121 | Vector | GACGCGCCCTGTAGCGGCGCATTAAGCGCGGCGGGTGTGGTGGTT |
| | GTM | ACGCGCAGCGTGACCGCTACACTTGCCAGCGCCCTAGCGCCCGCTC |
| | Plasmid | CTTTCGCTTTCTTCCCTTCCTTTCTCGCCACGTTCGCCGGCTTTCCCC |
| | | GTCAAGCTCTAAATCGGGGGCTCCCTTTAGGGTTCCGATTTAGTGC |
| | | TTTACGGCACCTCGACCCCAAAAAACTTGATTAGGGTGATGGTTCA |
| | | CGTAGTGGGCCATCGCCCTGATAGACGGTTTTTCGCCCTTTGACGT |
| | | TGGAGTCCACGTTCTTTAATAGTGGACTCTTGTTCCAAACTGGAAC |
| | | AACACTCAACCCTATCTCGGTCTATTCTTTTGATTTATAAGGGATTT |
| | | TGCCGATTTCGGCCTATTGGTTAAAAAATGAGCTGATTTAACAAAA |
| | | ATTTAACGCGAATTTTAACAAAATATTAACGTTTACAATTTCAGGT |
| | | GGCACTTTTCGGGGAAATGTGCGCGGAACCCCTATTTGTTTATTTT |
| | | TCTAAATACATTCAAATATGTATCCGCTCATGAGACAATAACCCTG |
| | | ATAAATGCTTCAATAATATTGAAAAAGGAAGAGTATGAGTATTCA |
| | | ACATTTCCGTGTCGCCCTTATTCCCTTTTTTGCGGCATTTTGCCTTC |
| | | CTGTTTTTGCTCACCCAGAAACGCTGGTGAAAGTAAAAGATGCTGA |
| | | AGATCAGTTGGGTGCACGAGTGGGTTACATCGAACTGGATCTCAA |
| | | CAGCGGTAAGATCCTTGAGAGTTTTCGCCCCGAAGAACGTTTTCCA |
| | | ATGATGAGCACTTTTAAAGTTCTGCTATGTGGCGCGGTATTATCCC |
| | | GTATTGACGCCGGGCAAGAGCAACTCGGTCGCCGCATACACTATT |
| | | CTCAGAATGACTTGGTTGAGTACTCACCAGTCACAGAAAAGCATCT |
| | | TACGGATGGCATGACAGTAAGAGAATTATGCAGTGCTGCCATAAC |
| | | CATGAGTGATAACACTGCGGCCAACTTACTTCTGACAACGATCGG |
| | | AGGACCGAAGGAGCTAACCGCTTTTTTGCACAACATGGGGGATCA |
| | | TGTAACTCGCCTTGATCGTTGGGAACCGGAGCTGAATGAAGCCAT |
| | | ACCAAACGACGAGCGTGACACCACGATGCCTGTAGCAATGGCAAC |
| | | AACGTTGCGCAAACTATTAACTGGCGAACTACTTACTCTAGCTTCC |
| | | CGGCAACAATTAATAGACTGGATGGAGGCGGATAAAGTTGCAGGA |
| | | CCACTTCTGCGCTCGGCCCTTCCGGCTGGCTGGTTTATTGCTGATA |
| | | AATCTGGAGCCGGTGAGCGTGGGTCTCGCGGTATCATTGCAGCACT |
| | | GGGGCCAGATGGTAAGCCCTCCCGTATCGTAGTTATCTACACGACG |
| | | GGGAGTCAGGCAACTATGGATGAACGAAATAGACAGATCGCTGAG |
| | | ATAGGTGCCTCACTGATTAAGCATTGGTAACTGTCAGACCAAGTTT |
| | | ACTCATATATACTTTAGATTGATTTAAAACTTCATTTTTAATTTAAA |
| | | AGGATCTAGGTGAAGATCCTTTTTGATAATCTCATGACCAAAATCC |
| | | CTTAACGTGAGTTTTCGTTCCACTGAGCGTCAGACCCCGTAGAAAA |
| | | GATCAAAGGATCTTCTTGAGATCCTTTTTTTCTGCGCGTAATCTGCT |
| | | GCTTGCAAACAAAAAAACCACCGCTACCAGCGGTGGTTTGTTTGCC |
| | | GGATCAAGAGCTACCAACTCTTTTTCCGAAGGTAACTGGCTTCAGC |
| | | AGAGCGCAGATACCAAATACTGTCCTTCTAGTGTAGCCGTAGTTAG |
| | | GCCACCACTTCAAGAACTCTGTAGCACCGCCTACATACCTCGCTCT |
| | | GCTAATCCTGTTACCAGTGGCTGCTGCCAGTGGCGATAAGTCGTGT |
| | | CTTACCGGGTTGGACTCAAGACGATAGTTACCGGATAAGGCGCAG |
| | | CGGTCGGGCTGAACGGGGGGTTCGTGCACACAGCCCAGCTTGGAG |
| | | CGAACGACCTACACCGAACTGAGATACCTACAGCGTGAGCATTGA |
| | | GAAAGCGCCACGCTTCCCGAAGGGAGAAAGGCGGACAGGTATCCG |
| | | GTAAGCGGCAGGGTCGGAACAGGAGAGCGCACGAGGGAGCTTCC |
| | | AGGGGGAAACGCCTGGTATCTTTATAGTCCTGTCGGGTTTCGCCAC |
| | | CTCTGACTTGAGCGTCGATTTTTGTGATGCTCGTCAGGGGGGCGGA |
| | | GCCTATGGAAAAACGCCAGCAACGCGGCCTTTTTACGGTTCCTGGC |
| | | CTTTTGCTGGCCTTTTGCTCACATGTTCTTTCCTGCGTTATCCCCTG |
| | | ATTCTGTGGATAACCGTATTACCGCCTTTGAGTGAGCTGATACCGC |
| | | TCGCCGCAGCCGAACGACCGAGCGCAGCGAGTCAGTGAGCGAGGA |
| | | AGCGGAAGAGCGCCTGATGCGGTATTTTCTCCTTACGCATCTGTGC |
| | | GGTATTTCACACCGCAGACCAGCCGCGTAACCTGGCAAAATCGGT |
| | | TACGGTTGAGTAATAAATGGATGCCCTGCGTAAGCGGGTGTGGGC |
| | | GGACAATAAAGTCTTAAACTGAACAAAATAGATCTAAACTATGAC |
| | | AATAAAGTCTTAAACTAGACAGAATAGTTGTAAACTGAAATCAGT |
| | | CCAGTTATGCTGTGAAAAAGCATACTGGACTTTTGTTATGGCTAAA |
| | | GCAAACTCTTCATTTTCTGAAGTGCAAATTGCCCGTCGTATTAAAG |
| | | AGGGGCGTGGCCAAGGGCATGGTAAAGACTATATTCGCGGCGTTG |
| | | TGACAATTTACCGAACAACTCCGCGGCCGGGAAGCCGATCTCGGC |
| | | TTGAACGAATTGTTAGGTGGCGGTACTTGGGTCGATATCAAAGTGC |
| | | ATCACTTCTTCCCGTATGCCCAACTTTGTATAGAGAGCCACTGCGG |
| | | GATCGTCACCGTAATCTGCTTGCACGTAGATCACATAAGCACCAAG |
| | | CGCGTTGGCCTCATGCTTGAGGAGATTGATGAGCGCGGTGGCAAT |
| | | GCCCTGCCTCCGGTGCTCGCCGGAGACTGCGAGATCATAGATATA |
| | | GATCTCACTACGCGGCTGCTCAAACCTGGGCAGAACGTAAGCCGC |
| | | GAGAGCGCCAACAACCGCTTCTTGGTCGAAGGCAGCAAGCGCGAT |
| | | GAATGTCTTACTACGGAGCAAGTTCCCGAGGTAATCGGAGTCCGG |
| | | CTGATGTTGGGAGTAGGTGGCTACGTCTCCGAACTCACGACCGAA |
| | | AAGATCAAGAGCAGCCCGCATGGATTTGACTTGGTCAGGGCCGAG |
| | | CCTACATGTGCGAATGATGCCCATACTTGAGCCACCTAACTTTGTT |
| | | TTAGGGCGACTGCCTTGCTGTGTAACGTCATTGCTGCTGCGTAACA |
| | | TCGTTGCTGCTCCATAACATCAAACATCGACCCACGGCGTAACGCG |
| | | CTTGCTGCTTGGATGCCCGAGGCATAGACTGTACAAAAAAACAGT |
| | | CATAACAAGCCATGAAAACCGCCACTGCGCCGTTACCACCGCTGC |
| | | GTTCGGTCAAGGTTCTGGACCAGTTGCGTGAGCGCATACGCTACTT |
| | | GCATTACAGTTTACGAACCGAACAGGCTTATGTCAACTGGGTTCGT |
| | | GCCTTCATCCGTTTCCACGGTGTGCGTCACCCGGCAACCTTGGGCA |
| | | GCAGCGAAGTCGAGGCATTTCTGTCCTGGCTGGCGAACGAGCGCA |
| | | AGGTTTCGGTCTCCACGCATCGTCAGGCATTGGCGGCCTTGCTGTT |
| | | CTTCTACGGCAAGGTGCTGTGCACGGATCTGCCCTGGCTTCAGGAG |
| | | ATCGGAAGACCTCGGCCGTCGCGGCGCTTGCCGGTGGTGCTGACC |
| | | CCGGATGAAGTGGTTCGCATCCTCGGTTTTCTGGAAGGCGAGCATC |
| | | GTTTGTTCGCCCAGGACTCTAGCTATAGTTCTAGTGGTTGGCTACA |
| | | GCTTGCTGCGCGCTCGCTCGCTCACTGAGGCCGCCCGGGCAAAGCC |
| | | CGGGCGTCGGGCGACCTTTGGTCGCCCGGCCTCAGTGAGCGAGCG |
| | | AGCGCGCAGAGAGGGAGTGGCCAACTCCATCACTAGGGGTTCCTA |
| | | GGAAGCTGATCTGAATTCGGTACCCGTTACATAACTTACGGTAAAT |
| | | GGCCCGCCTGGCTGACCGCCCAACGACCCCCGCCCATTGACGTCA |
| | | ATAATGACGTATGTTCCCATAGTAACGCCAATAGGGACTTTCCATT |
| | | GACGTCAATGGGTGGAGTATTTACGGTAAACTGCCCACTTGGCAGT |
| | | ACATCAAGTGTATCATATGCCAAGTACGCCCCCTATTGACGTCAAT |
| | | GACGGTAAATGGCCCGCCTGGCATTATGCCCAGTACATGACCTTAT |
| | | GGGACTTTCCTACTTGGCAGTACATCTACGTATTAGTCATCGCTAT |
| | | TACCATGGCTCGAGGTGAGCCCCACGTTCTGCTTCACTCTCCCCAT |
| | | CTCCCCCCCCTCCCCACCCCCAATTTTGTATTTATTTATTTTTTAATT |
| | | ATTTTGTGCAGCGATGGGGGCGGGGGGGGGGGGGGGGCGCGCGCC |
| | | AGGCGGGGCGGGGCGGGGCGAGGGGGGGGCGGGGCGAGGCGGA |
| | | GAGGTGCGGCGGCAGCCAATCAGAGCGGCGCGCTCCGAAAGTTTC |
| | | CTTTTATGGCGAGGCGGCGGCGGCGGCGGCCCTATAAAAAGCGAA |
| | | GCGCGCGGCGGGCGGGAGTCGCTGCGTTGCCTTCGCCCCGTGCCCC |
| | | GCTCCGCGCCGCCTCGCGCCGCCCGCCCCGGCTCTGACTGACCGCG |
| | | TTACTCCCACAGGTAAGTTTAGTCTTTTTGTCTTTTATTTCAGGTCC |
| | | CGGATCCGGTGGTGGTGCAAATCAAAGAACTGCTCCTCAGTGGAT |
| | | GTTGCCTTTACTTCTAGGCCTGCCGCCACCATGGGAATCCAAGGAG |
| | | GGTCTGTCCTGTTCGGGCTGCTGCTCGTCCTGGCTGTCTTCTGCCAT |
| | | TCAGGTCATAGCCTGCAGTGCTACAACTGTCCTAACCCAACTGCTG |
| | | ACTGCAAAACAGCCGTCAATTGTTCATCTGATTTTGATGCGTGTCT |
| | | CATTACCAAAGCTGGGTTACAAGTGTATAACAAGTGTTGGAAGTTT |
| | | GAGCATTGCAATTTCAACGACGTCACAACCCGCTTGAGGGAAAAT |
| | | GAGCTAACGTACTACTGCTGCAAGAAGGACCTGTGTAACTTTAAC |
| | | GAACAGCTTGAAAATGGTGGGACATCCTTATCAGAGAAAACAGTT |
| | | CTTCTGCTGGTGACTCCATTTCTGGCAGCAGCCTGGAGCCTTCATC |
| | | CCTAAAGAACCAGGTGAGCCTGACCTGCCTGGTGAAGGGCTTCTA |
| | | CCCCAGCGACATCGCCGTGGAGTGGGAGAGCAACGGCCAGCCCGA |
| | | GAACAACTACAAGACCACCCCCCCCGTGCTGGACAGCGACGGCAG |
| | | CTTCTTCCTGTACAGCAGACTGACCGTGGACAAGAGCAGATGGCA |
| | | GGAGGGCAACGTGTTCAGCTGCAGCGTGATGCACGAGGCCCTGCA |
| | | CAACCACTACACCCAGAAGAGCCTGAGCCTGAGCCTGGGCAAGGG |
| | | CGGAGGCGGAAGCGGCGGAGGCGGATCTGGCGGAGGCGGAAGCG |
| | | GCGGAGGCGGCTCTGAGCACCCCAACGCGCGCAAATACAAAGGAG |
| | | CCAACAAGAAGGGCTTGTCCAAGGGCTGCTTCGGCCTCAAGCTGG |
| | | ACCGAATCGGCTCCATGAGCGGCCTGGGATGTTAACTCGAGAATC |
| | | AACCTCTGGATTACAAAATTTGTGAAAGATTGACTGGTATTCTTAA |
| | | CTATGTTGCTCCTTTTACGCTATGTGGATACGCTGCTTTAATGCCTT |
| | | TGTATCATGCTATTGCTTCCCGTATGGCTTTCATTTTCTCCTCCTTGT |
| | | ATAAATCCTGGTTGCTGTCTCTTTATGAGGAGTTGTGGCCCGTTGT |
| | | CAGGCAACGTGGCGTGGTGTGCACTGTGTTTGCTGACGCAACCCCC |
| | | ACTGGTTGGGGCATTGCCACCACCTGTCAGCTCCTTTCCGGGACTT |
| | | TCGCTTTCCCCCTCCCTATTGCCACGGCGGAACTCATCGCCGCCTG |
| | | CCTTGCCCGCTGCTGGACAGGGGCTCGGCTGTTGGGCACTGACAAT |
| | | TCCGTGGTGGTACCTTCGAAAATAAAATATCTTTATTTTCATTACAT |
| | | CTGTGTGTTGGTTTTTTGTGTGGCATGCTGGGGAGAGATCAACCCC |
| | | ACTCCCTCTCTGCGCGCTCGCTCGCTCACTGAGGCCGGGCGACCAA |
| | | AGGTCGCCCGACGCCCGGGCTTTGCCCGGGCGGCCTCAGTGAGCG |
| | | AGCGAGCGCGCAGGATCCGAGCTTGTCGAGAAGTACTAGAGGATC |
| | | ATAATCAGCCATACCACATTTGTAGAGGTTTTACTTGCTTTAAAAA |
| | | ACCTCCCACACCTCCCCCTGAACCTGAAACATAAAATGAATGCAAT |
| | | TGTTGTTGTTAACTTGTTTATTGCAGCTTATAATGGTTACAAATAAA |
| | | GCAATAGCATCACAAATTTCACAAATAAAGCATTTTTTTCACTGCA |
| | | TTCTAGTTGTGGTTTGTCCAAACTCATCAATGTATCTTATCATGTCT |
| | | GGATCTGATCACTGCTTGAGCCTAGGAGATCCGAACCAGATAAGT |
| | | GAAATCTAGTTCCAAACTATTTTGTCATTTTTAATTTTCGTATTAGC |
| | | TTACGACGCTACACCCAGTTCCCATCTATTTTGTCACTCTTCCCTAA |
| | | ATAATCCTTAAAAACTCCATTTCCACCCCTCCCAGTTCCCAACTATT |
| | | TTGTCCGCCCACAGCGGGGCATTTTTCTTCCTGTTATGTTTTTAATC |
| | | AAACATCCTGCCAACTCCATGTGACAAACCGTCATCTTCGGCTACT |
| | | TTTTCTCTGTCACAGAATGAAAATTTTTCTGTCATCTCTTCGTTATT |
| | | AATGTTTGTAATTGACTGAATATCAACGCTTATTTGCAGCCTGAAT |
| | | GGCGAATG |
| 122 | Vector | GACGCGCCCTGTAGCGGCGCATTAAGCGCGGCGGGTGTGGTGGTT |
| | GTP | ACGCGCAGCGTGACCGCTACACTTGCCAGCGCCCTAGCGCCCGCTC |
| | Plasmid | CTTTCGCTTTCTTCCCTTCCTTTCTCGCCACGTTCGCCGGCTTTCCCC |
| | | GTCAAGCTCTAAATCGGGGGCTCCCTTTAGGGTTCCGATTTAGTGC |
| | | TTTACGGCACCTCGACCCCAAAAAACTTGATTAGGGTGATGGTTCA |
| | | CGTAGTGGGCCATCGCCCTGATAGACGGTTTTTCGCCCTTTGACGT |
| | | TGGAGTCCACGTTCTTTAATAGTGGACTCTTGTTCCAAACTGGAAC |
| | | AACACTCAACCCTATCTCGGTCTATTCTTTTGATTTATAAGGGATTT |
| | | TGCCGATTTCGGCCTATTGGTTAAAAAATGAGCTGATTTAACAAAA |
| | | ATTTAACGCGAATTTTAACAAAATATTAACGTTTACAATTTCAGGT |
| | | GGCACTTTTCGGGGAAATGTGCGCGGAACCCCTATTTGTTTATTTT |
| | | TCTAAATACATTCAAATATGTATCCGCTCATGAGACAATAACCCTG |
| | | ATAAATGCTTCAATAATATTGAAAAAGGAAGAGTATGAGTATTCA |
| | | ACATTTCCGTGTCGCCCTTATTCCCTTTTTTGCGGCATTTTGCCTTC |
| | | CTGTTTTTGCTCACCCAGAAACGCTGGTGAAAGTAAAAGATGCTGA |
| | | AGATCAGTTGGGTGCACGAGTGGGTTACATCGAACTGGATCTCAA |
| | | CAGCGGTAAGATCCTTGAGAGTTTTCGCCCCGAAGAACGTTTTCCA |
| | | ATGATGAGCACTTTTAAAGTTCTGCTATGTGGCGCGGTATTATCCC |
| | | GTATTGACGCCGGGCAAGAGCAACTCGGTCGCCGCATACACTATT |
| | | CTCAGAATGACTTGGTTGAGTACTCACCAGTCACAGAAAAGCATCT |
| | | TACGGATGGCATGACAGTAAGAGAATTATGCAGTGCTGCCATAAC |
| | | CATGAGTGATAACACTGCGGCCAACTTACTTCTGACAACGATCGG |
| | | AGGACCGAAGGAGCTAACCGCTTTTTTGCACAACATGGGGGATCA |
| | | TGTAACTCGCCTTGATCGTTGGGAACCGGAGCTGAATGAAGCCAT |
| | | ACCAAACGACGAGCGTGACACCACGATGCCTGTAGCAATGGCAAC |
| | | AACGTTGCGCAAACTATTAACTGGCGAACTACTTACTCTAGCTTCC |
| | | CGGCAACAATTAATAGACTGGATGGAGGCGGATAAAGTTGCAGGA |
| | | CCACTTCTGCGCTCGGCCCTTCCGGCTGGCTGGTTTATTGCTGATA |
| | | AATCTGGAGCCGGTGAGCGTGGGTCTCGCGGTATCATTGCAGCACT |
| | | GGGGCCAGATGGTAAGCCCTCCCGTATCGTAGTTATCTACACGACG |
| | | GGGAGTCAGGCAACTATGGATGAACGAAATAGACAGATCGCTGAG |
| | | ATAGGTGCCTCACTGATTAAGCATTGGTAACTGTCAGACCAAGTTT |
| | | ACTCATATATACTTTAGATTGATTTAAAACTTCATTTTTAATTTAAA |
| | | AGGATCTAGGTGAAGATCCTTTTTGATAATCTCATGACCAAAATCC |
| | | CTTAACGTGAGTTTTCGTTCCACTGAGCGTCAGACCCCGTAGAAAA |
| | | GATCAAAGGATCTTCTTGAGATCCTTTTTTTCTGCGCGTAATCTGCT |
| | | GCTTGCAAACAAAAAAACCACCGCTACCAGCGGTGGTTTGTTTGCC |
| | | GGATCAAGAGCTACCAACTCTTTTTCCGAAGGTAACTGGCTTCAGC |
| | | AGAGCGCAGATACCAAATACTGTCCTTCTAGTGTAGCCGTAGTTAG |
| | | GCCACCACTTCAAGAACTCTGTAGCACCGCCTACATACCTCGCTCT |
| | | GCTAATCCTGTTACCAGTGGCTGCTGCCAGTGGCGATAAGTCGTGT |
| | | CTTACCGGGTTGGACTCAAGACGATAGTTACCGGATAAGGCGCAG |
| | | CGGTCGGGCTGAACGGGGGGTTCGTGCACACAGCCCAGCTTGGAG |
| | | CGAACGACCTACACCGAACTGAGATACCTACAGCGTGAGCATTGA |
| | | GAAAGCGCCACGCTTCCCGAAGGGAGAAAGGCGGACAGGTATCCG |
| | | GTAAGCGGCAGGGTCGGAACAGGAGAGCGCACGAGGGAGCTTCC |
| | | AGGGGGAAACGCCTGGTATCTTTATAGTCCTGTCGGGTTTCGCCAC |
| | | CTCTGACTTGAGCGTCGATTTTTGTGATGCTCGTCAGGGGGGCGGA |
| | | GCCTATGGAAAAACGCCAGCAACGCGGCCTTTTTACGGTTCCTGGC |
| | | CTTTTGCTGGCCTTTTGCTCACATGTTCTTTCCTGCGTTATCCCCTG |
| | | ATTCTGTGGATAACCGTATTACCGCCTTTGAGTGAGCTGATACCGC |
| | | TCGCCGCAGCCGAACGACCGAGCGCAGCGAGTCAGTGAGCGAGGA |
| | | AGCGGAAGAGCGCCTGATGCGGTATTTTCTCCTTACGCATCTGTGC |
| | | GGTATTTCACACCGCAGACCAGCCGCGTAACCTGGCAAAATCGGT |
| | | TACGGTTGAGTAATAAATGGATGCCCTGCGTAAGCGGGTGTGGGC |
| | | GGACAATAAAGTCTTAAACTGAACAAAATAGATCTAAACTATGAC |
| | | AATAAAGTCTTAAACTAGACAGAATAGTTGTAAACTGAAATCAGT |
| | | CCAGTTATGCTGTGAAAAAGCATACTGGACTTTTGTTATGGCTAAA |
| | | GCAAACTCTTCATTTTCTGAAGTGCAAATTGCCCGTCGTATTAAAG |
| | | AGGGGCGTGGCCAAGGGCATGGTAAAGACTATATTCGCGGCGTTG |
| | | TGACAATTTACCGAACAACTCCGCGGCCGGGAAGCCGATCTCGGC |
| | | TTGAACGAATTGTTAGGTGGCGGTACTTGGGTCGATATCAAAGTGC |
| | | ATCACTTCTTCCCGTATGCCCAACTTTGTATAGAGAGCCACTGCGG |
| | | GATCGTCACCGTAATCTGCTTGCACGTAGATCACATAAGCACCAAG |
| | | CGCGTTGGCCTCATGCTTGAGGAGATTGATGAGCGCGGTGGCAAT |
| | | GCCCTGCCTCCGGTGCTCGCCGGAGACTGCGAGATCATAGATATA |
| | | GATCTCACTACGCGGCTGCTCAAACCTGGGCAGAACGTAAGCCGC |
| | | GAGAGCGCCAACAACCGCTTCTTGGTCGAAGGCAGCAAGCGCGAT |
| | | GAATGTCTTACTACGGAGCAAGTTCCCGAGGTAATCGGAGTCCGG |
| | | CTGATGTTGGGAGTAGGTGGCTACGTCTCCGAACTCACGACCGAA |
| | | AAGATCAAGAGCAGCCCGCATGGATTTGACTTGGTCAGGGCCGAG |
| | | CCTACATGTGCGAATGATGCCCATACTTGAGCCACCTAACTTTGTT |
| | | TTAGGGCGACTGCCTTGCTGTGTAACGTCATTGCTGCTGCGTAACA |
| | | TCGTTGCTGCTCCATAACATCAAACATCGACCCACGGCGTAACGCG |
| | | CTTGCTGCTTGGATGCCCGAGGCATAGACTGTACAAAAAAACAGT |
| | | CATAACAAGCCATGAAAACCGCCACTGCGCCGTTACCACCGCTGC |
| | | GTTCGGTCAAGGTTCTGGACCAGTTGCGTGAGCGCATACGCTACTT |
| | | GCATTACAGTTTACGAACCGAACAGGCTTATGTCAACTGGGTTCGT |
| | | GCCTTCATCCGTTTCCACGGTGTGCGTCACCCGGCAACCTTGGGCA |
| | | GCAGCGAAGTCGAGGCATTTCTGTCCTGGCTGGCGAACGAGCGCA |
| | | AGGTTTCGGTCTCCACGCATCGTCAGGCATTGGCGGCCTTGCTGTT |
| | | CTTCTACGGCAAGGTGCTGTGCACGGATCTGCCCTGGCTTCAGGAG |
| | | ATCGGAAGACCTCGGCCGTCGCGGCGCTTGCCGGTGGTGCTGACC |
| | | CCGGATGAAGTGGTTCGCATCCTCGGTTTTCTGGAAGGCGAGCATC |
| | | GTTTGTTCGCCCAGGACTCTAGCTATAGTTCTAGTGGTTGGCTACA |
| | | GCTTGCTGCGCGCTCGCTCGCTCACTGAGGCCGCCCGGGCAAAGCC |
| | | CGGGCGTCGGGCGACCTTTGGTCGCCCGGCCTCAGTGAGCGAGCG |
| | | AGCGCGCAGAGAGGGAGTGGCCAACTCCATCACTAGGGGTTCCTA |
| | | GGAAGCTGATCTGAATTCGGTACCCGTTACATAACTTACGGTAAAT |
| | | GGCCCGCCTGGCTGACCGCCCAACGACCCCCGCCCATTGACGTCA |
| | | ATAATGACGTATGTTCCCATAGTAACGCCAATAGGGACTTTCCATT |
| | | GACGTCAATGGGTGGAGTATTTACGGTAAACTGCCCACTTGGCAGT |
| | | ACATCAAGTGTATCATATGCCAAGTACGCCCCCTATTGACGTCAAT |
| | | GACGGTAAATGGCCCGCCTGGCATTATGCCCAGTACATGACCTTAT |
| | | GGGACTTTCCTACTTGGCAGTACATCTACGTATTAGTCATCGCTAT |
| | | TACCATGGCTCGAGGTGAGCCCCACGTTCTGCTTCACTCTCCCCAT |
| | | CTCCCCCCCCTCCCCACCCCCAATTTTGTATTTATTTATTTTTTAATT |
| | | ATTTTGTGCAGCGATGGGGGCGGGGGGGGGGGGGGGGCGCGCGCC |
| | | AGGCGGGGCGGGGCGGGGCGAGGGGCGGGGCGGGGCGAGGCGGA |
| | | GAGGTGCGGCGGCAGCCAATCAGAGCGGCGCGCTCCGAAAGTTTC |
| | | CTTTTATGGCGAGGCGGCGGCGGCGGCGGCCCTATAAAAAGCGAA |
| | | GCGCGCGGCGGGCGGGAGTCGCTGCGTTGCCTTCGCCCCGTGCCCC |
| | | GCTCCGCGCCGCCTCGCGCCGCCCGCCCCGGCTCTGACTGACCGCG |
| | | TTACTCCCACAGGTAAGTTTAGTCTTTTTGTCTTTTATTTCAGGTCC |
| | | CGGATCCGGTGGTGGTGCAAATCAAAGAACTGCTCCTCAGTGGAT |
| | | GTTGCCTTTACTTCTAGGCCTGCCGCCACCATGGGCATCCAGGGCG |
| | | GCAGCGTGCTGTTCGGCCTGCTGCTGGTGCTGGCCGTGTTCTGCCA |
| | | CAGCGGCCACAGCCTGCAGTGCTACAACTGCCCCAACCCCACCGC |
| | | CGACTGCAAGACCGCCGTGAACTGCAGCAGCGACTTCGACGCCTG |
| | | CCTGATCACCAAGGCCGGCCTGCAGGTGTACAACAAGTGCTGGAA |
| | | GTTCGAGCACTGCAACTTCAACGACGTGACCACCAGGCTGAGGGA |
| | | GAACGAGCTGACCTACTACTGCTGCAAGAAGGACCTGTGCAACTT |
| | | CAACGAGCAGCTGGAGAACGGCGGCACCAGCCTGAGCGAGAAGA |
| | | CCGTGCTGCTGCTGGTGACCCCCTTCCTGGCCGCCGCCTGGAGCCT |
| | | GCACCCCTGAAGAACCAGGTGAGCCTGACCTGCCTGGTGAAGGGC |
| | | TTCTACCCCAGCGACATCGCCGTGGAGTGGGAGAGCAACGGCCAG |
| | | CCCGAGAACAACTACAAGACCACCCCCCCCGTGCTGGACAGCGAC |
| | | GGCAGCTTCTTCCTGTACAGCAGACTGACCGTGGACAAGAGCAGA |
| | | TGGCAGGAGGGCAACGTGTTCAGCTGCAGCGTGATGCACGAGGCC |
| | | CTGCACAACCACTACACCCAGAAGAGCCTGAGCCTGAGCCTGGGC |
| | | AAGGGCGGAGGCGGAAGCGGCGGAGGCGGATCTGGCGGAGGCGG |
| | | AAGCGGCGGAGGCGGCTCTGAGCACCCCAACGCGCGCAAATACAA |
| | | AGGAGCCAACAAGAAGGGCTTGTCCAAGGGCTGCTTCGGCCTCAA |
| | | GCTGGACCGAATCGGCTCCATGAGCGGCCTGGGATGTTAACTCGA |
| | | GAATCAACCTCTGGATTACAAAATTTGTGAAAGATTGACTGGTATT |
| | | CTTAACTATGTTGCTCCTTTTACGCTATGTGGATACGCTGCTTTAAT |
| | | GCCTTTGTATCATGCTATTGCTTCCCGTATGGCTTTCATTTTCTCCT |
| | | CCTTGTATAAATCCTGGTTGCTGTCTCTTTATGAGGAGTTGTGGCCC |
| | | GTTGTCAGGCAACGTGGCGTGGTGTGCACTGTGTTTGCTGACGCAA |
| | | CCCCCACTGGTTGGGGCATTGCCACCACCTGTCAGCTCCTTTCCGG |
| | | GACTTTCGCTTTCCCCCTCCCTATTGCCACGGCGGAACTCATCGCC |
| | | GCCTGCCTTGCCCGCTGCTGGACAGGGGCTCGGCTGTTGGGCACTG |
| | | ACAATTCCGTGGTGGTACCTTCGAAAATAAAATATCTTTATTTTCA |
| | | TTACATCTGTGTGTTGGTTTTTTGTGTGGCATGCTGGGGAGAGATC |
| | | AACCCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGGATCCGAGCTTGTCGAGAAGTACTAG |
| | | AGGATCATAATCAGCCATACCACATTTGTAGAGGTTTTACTTGCTT |
| | | TAAAAAACCTCCCACACCTCCCCCTGAACCTGAAACATAAAATGA |
| | | ATGCAATTGTTGTTGTTAACTTGTTTATTGCAGCTTATAATGGTTAC |
| | | AAATAAAGCAATAGCATCACAAATTTCACAAATAAAGCATTTTTTT |
| | | CACTGCATTCTAGTTGTGGTTTGTCCAAACTCATCAATGTATCTTAT |
| | | CATGTCTGGATCTGATCACTGCTTGAGCCTAGGAGATCCGAACCAG |
| | | ATAAGTGAAATCTAGTTCCAAACTATTTTGTCATTTTTAATTTTCGT |
| | | ATTAGCTTACGACGCTACACCCAGTTCCCATCTATTTTGTCACTCTT |
| | | CCCTAAATAATCCTTAAAAACTCCATTTCCACCCCTCCCAGTTCCC |
| | | AACTATTTTGTCCGCCCACAGCGGGGCATTTTTCTTCCTGTTATGTT |
| | | TTTAATCAAACATCCTGCCAACTCCATGTGACAAACCGTCATCTTC |
| | | GGCTACTTTTTCTCTGTCACAGAATGAAAATTTTTCTGTCATCTCTT |
| | | CGTTATTAATGTTTGTAATTGACTGAATATCAACGCTTATTTGCAG |
| | | CCTGAATGGCGAATG |
| 123 | Vector | GACGCGCCCTGTAGCGGCGCATTAAGCGCGGCGGGTGTGGTGGTT |
| | GTQ | ACGCGCAGCGTGACCGCTACACTTGCCAGCGCCCTAGCGCCCGCTC |
| | Plasmid | CTTTCGCTTTCTTCCCTTCCTTTCTCGCCACGTTCGCCGGCTTTCCCC |
| | | GTCAAGCTCTAAATCGGGGGCTCCCTTTAGGGTTCCGATTTAGTGC |
| | | TTTACGGCACCTCGACCCCAAAAAACTTGATTAGGGTGATGGTTCA |
| | | CGTAGTGGGCCATCGCCCTGATAGACGGTTTTTCGCCCTTTGACGT |
| | | TGGAGTCCACGTTCTTTAATAGTGGACTCTTGTTCCAAACTGGAAC |
| | | AACACTCAACCCTATCTCGGTCTATTCTTTTGATTTATAAGGGATTT |
| | | TGCCGATTTCGGCCTATTGGTTAAAAAATGAGCTGATTTAACAAAA |
| | | ATTTAACGCGAATTTTAACAAAATATTAACGTTTACAATTTCAGGT |
| | | GGCACTTTTCGGGGAAATGTGCGCGGAACCCCTATTTGTTTATTTT |
| | | TCTAAATACATTCAAATATGTATCCGCTCATGAGACAATAACCCTG |
| | | ATAAATGCTTCAATAATATTGAAAAAGGAAGAGTATGAGTATTCA |
| | | ACATTTCCGTGTCGCCCTTATTCCCTTTTTTGCGGCATTTTGCCTTC |
| | | CTGTTTTTGCTCACCCAGAAACGCTGGTGAAAGTAAAAGATGCTGA |
| | | AGATCAGTTGGGTGCACGAGTGGGTTACATCGAACTGGATCTCAA |
| | | CAGCGGTAAGATCCTTGAGAGTTTTCGCCCCGAAGAACGTTTTCCA |
| | | ATGATGAGCACTTTTAAAGTTCTGCTATGTGGCGCGGTATTATCCC |
| | | GTATTGACGCCGGGCAAGAGCAACTCGGTCGCCGCATACACTATT |
| | | CTCAGAATGACTTGGTTGAGTACTCACCAGTCACAGAAAAGCATCT |
| | | TACGGATGGCATGACAGTAAGAGAATTATGCAGTGCTGCCATAAC |
| | | CATGAGTGATAACACTGCGGCCAACTTACTTCTGACAACGATCGG |
| | | AGGACCGAAGGAGCTAACCGCTTTTTTGCACAACATGGGGGATCA |
| | | TGTAACTCGCCTTGATCGTTGGGAACCGGAGCTGAATGAAGCCAT |
| | | ACCAAACGACGAGCGTGACACCACGATGCCTGTAGCAATGGCAAC |
| | | AACGTTGCGCAAACTATTAACTGGCGAACTACTTACTCTAGCTTCC |
| | | CGGCAACAATTAATAGACTGGATGGAGGCGGATAAAGTTGCAGGA |
| | | CCACTTCTGCGCTCGGCCCTTCCGGCTGGCTGGTTTATTGCTGATA |
| | | AATCTGGAGCCGGTGAGCGTGGGTCTCGCGGTATCATTGCAGCACT |
| | | GGGGCCAGATGGTAAGCCCTCCCGTATCGTAGTTATCTACACGACG |
| | | GGGAGTCAGGCAACTATGGATGAACGAAATAGACAGATCGCTGAG |
| | | ATAGGTGCCTCACTGATTAAGCATTGGTAACTGTCAGACCAAGTTT |
| | | ACTCATATATACTTTAGATTGATTTAAAACTTCATTTTTAATTTAAA |
| | | AGGATCTAGGTGAAGATCCTTTTTGATAATCTCATGACCAAAATCC |
| | | CTTAACGTGAGTTTTCGTTCCACTGAGCGTCAGACCCCGTAGAAAA |
| | | GATCAAAGGATCTTCTTGAGATCCTTTTTTTCTGCGCGTAATCTGCT |
| | | GCTTGCAAACAAAAAAACCACCGCTACCAGCGGTGGTTTGTTTGCC |
| | | GGATCAAGAGCTACCAACTCTTTTTCCGAAGGTAACTGGCTTCAGC |
| | | AGAGCGCAGATACCAAATACTGTCCTTCTAGTGTAGCCGTAGTTAG |
| | | GCCACCACTTCAAGAACTCTGTAGCACCGCCTACATACCTCGCTCT |
| | | GCTAATCCTGTTACCAGTGGCTGCTGCCAGTGGCGATAAGTCGTGT |
| | | CTTACCGGGTTGGACTCAAGACGATAGTTACCGGATAAGGCGCAG |
| | | CGGTCGGGCTGAACGGGGGGTTCGTGCACACAGCCCAGCTTGGAG |
| | | CGAACGACCTACACCGAACTGAGATACCTACAGCGTGAGCATTGA |
| | | GAAAGCGCCACGCTTCCCGAAGGGAGAAAGGCGGACAGGTATCCG |
| | | GTAAGCGGCAGGGTCGGAACAGGAGAGCGCACGAGGGAGCTTCC |
| | | AGGGGGAAACGCCTGGTATCTTTATAGTCCTGTCGGGTTTCGCCAC |
| | | CTCTGACTTGAGCGTCGATTTTTGTGATGCTCGTCAGGGGGGCGGA |
| | | GCCTATGGAAAAACGCCAGCAACGCGGCCTTTTTACGGTTCCTGGC |
| | | CTTTTGCTGGCCTTTTGCTCACATGTTCTTTCCTGCGTTATCCCCTG |
| | | ATTCTGTGGATAACCGTATTACCGCCTTTGAGTGAGCTGATACCGC |
| | | TCGCCGCAGCCGAACGACCGAGCGCAGCGAGTCAGTGAGCGAGGA |
| | | AGCGGAAGAGCGCCTGATGCGGTATTTTCTCCTTACGCATCTGTGC |
| | | GGTATTTCACACCGCAGACCAGCCGCGTAACCTGGCAAAATCGGT |
| | | TACGGTTGAGTAATAAATGGATGCCCTGCGTAAGCGGGTGTGGGC |
| | | GGACAATAAAGTCTTAAACTGAACAAAATAGATCTAAACTATGAC |
| | | AATAAAGTCTTAAACTAGACAGAATAGTTGTAAACTGAAATCAGT |
| | | CCAGTTATGCTGTGAAAAAGCATACTGGACTTTTGTTATGGCTAAA |
| | | GCAAACTCTTCATTTTCTGAAGTGCAAATTGCCCGTCGTATTAAAG |
| | | AGGGGCGTGGCCAAGGGCATGGTAAAGACTATATTCGCGGCGTTG |
| | | TGACAATTTACCGAACAACTCCGCGGCCGGGAAGCCGATCTCGGC |
| | | TTGAACGAATTGTTAGGTGGCGGTACTTGGGTCGATATCAAAGTGC |
| | | ATCACTTCTTCCCGTATGCCCAACTTTGTATAGAGAGCCACTGCGG |
| | | GATCGTCACCGTAATCTGCTTGCACGTAGATCACATAAGCACCAAG |
| | | CGCGTTGGCCTCATGCTTGAGGAGATTGATGAGCGCGGTGGCAAT |
| | | GCCCTGCCTCCGGTGCTCGCCGGAGACTGCGAGATCATAGATATA |
| | | GATCTCACTACGCGGCTGCTCAAACCTGGGCAGAACGTAAGCCGC |
| | | GAGAGCGCCAACAACCGCTTCTTGGTCGAAGGCAGCAAGCGCGAT |
| | | GAATGTCTTACTACGGAGCAAGTTCCCGAGGTAATCGGAGTCCGG |
| | | CTGATGTTGGGAGTAGGTGGCTACGTCTCCGAACTCACGACCGAA |
| | | AAGATCAAGAGCAGCCCGCATGGATTTGACTTGGTCAGGGCCGAG |
| | | CCTACATGTGCGAATGATGCCCATACTTGAGCCACCTAACTTTGTT |
| | | TTAGGGCGACTGCCTTGCTGTGTAACGTCATTGCTGCTGCGTAACA |
| | | TCGTTGCTGCTCCATAACATCAAACATCGACCCACGGCGTAACGCG |
| | | CTTGCTGCTTGGATGCCCGAGGCATAGACTGTACAAAAAAACAGT |
| | | CATAACAAGCCATGAAAACCGCCACTGCGCCGTTACCACCGCTGC |
| | | GTTCGGTCAAGGTTCTGGACCAGTTGCGTGAGCGCATACGCTACTT |
| | | GCATTACAGTTTACGAACCGAACAGGCTTATGTCAACTGGGTTCGT |
| | | GCCTTCATCCGTTTCCACGGTGTGCGTCACCCGGCAACCTTGGGCA |
| | | GCAGCGAAGTCGAGGCATTTCTGTCCTGGCTGGCGAACGAGCGCA |
| | | AGGTTTCGGTCTCCACGCATCGTCAGGCATTGGCGGCCTTGCTGTT |
| | | CTTCTACGGCAAGGTGCTGTGCACGGATCTGCCCTGGCTTCAGGAG |
| | | ATCGGAAGACCTCGGCCGTCGCGGCGCTTGCCGGTGGTGCTGACC |
| | | CCGGATGAAGTGGTTCGCATCCTCGGTTTTCTGGAAGGCGAGCATC |
| | | GTTTGTTCGCCCAGGACTCTAGCTATAGTTCTAGTGGTTGGCTACA |
| | | GCTTGCTGCGCGCTCGCTCGCTCACTGAGGCCGCCCGGGCAAAGCC |
| | | CGGGCGTCGGGCGACCTTTGGTCGCCCGGCCTCAGTGAGCGAGCG |
| | | AGCGCGCAGAGAGGGAGTGGCCAACTCCATCACTAGGGGTTCCTA |
| | | GGAAGCTGATCTGAATTCGGTACCCGTTACATAACTTACGGTAAAT |
| | | GGCCCGCCTGGCTGACCGCCCAACGACCCCCGCCCATTGACGTCA |
| | | ATAATGACGTATGTTCCCATAGTAACGCCAATAGGGACTTTCCATT |
| | | GACGTCAATGGGTGGAGTATTTACGGTAAACTGCCCACTTGGCAGT |
| | | ACATCAAGTGTATCATATGCCAAGTACGCCCCCTATTGACGTCAAT |
| | | GACGGTAAATGGCCCGCCTGGCATTATGCCCAGTACATGACCTTAT |
| | | GGGACTTTCCTACTTGGCAGTACATCTACGTATTAGTCATCGCTAT |
| | | TACCATGGCTCGAGGTGAGCCCCACGTTCTGCTTCACTCTCCCCAT |
| | | CTCCCCCCCCTCCCCACCCCCAATTTTGTATTTATTTATTTTTTAATT |
| | | ATTTTGTGCAGCGATGGGGGCGGGGGGGGGGGGGGGGCGCGCGCC |
| | | AGGCGGGGCGGGGCGGGGCGAGGGGCGGGGCGGGGCGAGGCGGA |
| | | GAGGTGCGGCGGCAGCCAATCAGAGCGGCGCGCTCCGAAAGTTTC |
| | | CTTTTATGGCGAGGCGGCGGCGGCGGCGGCCCTATAAAAAGCGAA |
| | | GCGCGCGGCGGGCGGGAGTCGCTGCGTTGCCTTCGCCCCGTGCCCC |
| | | GCTCCGCGCCGCCTCGCGCCGCCCGCCCCGGCTCTGACTGACCGCG |
| | | TTACTCCCACAGGTAAGTTTAGTCTTTTTGTCTTTTATTTCAGGTCC |
| | | CGGATCCGGTGGTGGTGCAAATCAAAGAACTGCTCCTCAGTGGAT |
| | | GTTGCCTTTACTTCTAGGCCTGCCGCCACCATGGGCATCCAGGGCG |
| | | GCAGCGTGCTGTTCGGCCTGCTGCTGGTGCTGGCCGTGTTCTGCCA |
| | | CAGCGGCCACAGCCTGCAGTGCTACAACTGCCCCAACCCCACCGC |
| | | CGACTGCAAGACCGCCGTGAACTGCAGCAGCGACTTCGACGCCTG |
| | | CCTGATCACCAAGGCCGGCCTGCAGGTGTACAACAAGTGCTGGAA |
| | | GTTCGAGCACTGCAACTTCAACGACGTGACCACCCGCCTGCGCGA |
| | | GAACGAGCTGACCTACTACTGCTGCAAGAAGGACCTGTGCAACTT |
| | | CAACGAGCAGCTGGAGAACGGCGGCACCAGCCTGAGCGAGAAGA |
| | | CCGTGCTGCTGCTGGTGACCCCCTTCCTGGCCGCCGCCTGGAGCCT |
| | | GCACCCCTAAAGAACCAGGTGAGCCTGACCTGCCTGGTGAAGGGC |
| | | TTCTACCCCAGCGACATCGCCGTGGAGTGGGAGAGCAACGGCCAG |
| | | CCCGAGAACAACTACAAGACCACCCCCCCCGTGCTGGACAGCGAC |
| | | GGCAGCTTCTTCCTGTACAGCAGACTGACCGTGGACAAGAGCAGA |
| | | TGGCAGGAGGGCAACGTGTTCAGCTGCAGCGTGATGCACGAGGCC |
| | | CTGCACAACCACTACACCCAGAAGAGCCTGAGCCTGAGCCTGGGC |
| | | AAGGGCGGAGGCGGAAGCGGCGGAGGCGGATCTGGCGGAGGCGG |
| | | AAGCGGCGGAGGCGGCTCTGAGCACCCCAACGCGCGCAAATACAA |
| | | AGGAGCCAACAAGAAGGGCTTGTCCAAGGGCTGCTTCGGCCTCAA |
| | | GCTGGACCGAATCGGCTCCATGAGCGGCCTGGGATGTTAACTCGA |
| | | GAATCAACCTCTGGATTACAAAATTTGTGAAAGATTGACTGGTATT |
| | | CTTAACTATGTTGCTCCTTTTACGCTATGTGGATACGCTGCTTTAAT |
| | | GCCTTTGTATCATGCTATTGCTTCCCGTATGGCTTTCATTTTCTCCT |
| | | CCTTGTATAAATCCTGGTTGCTGTCTCTTTATGAGGAGTTGTGGCCC |
| | | GTTGTCAGGCAACGTGGCGTGGTGTGCACTGTGTTTGCTGACGCAA |
| | | CCCCCACTGGTTGGGGCATTGCCACCACCTGTCAGCTCCTTTCCGG |
| | | GACTTTCGCTTTCCCCCTCCCTATTGCCACGGCGGAACTCATCGCC |
| | | GCCTGCCTTGCCCGCTGCTGGACAGGGGCTCGGCTGTTGGGCACTG |
| | | ACAATTCCGTGGTGGTACCTTCGAAAATAAAATATCTTTATTTTCA |
| | | TTACATCTGTGTGTTGGTTTTTTGTGTGGCATGCTGGGGAGAGATC |
| | | AACCCCACTCCCTCTCTGCGCGCTCGCTCGCTCACTGAGGCCGGGC |
| | | GACCAAAGGTCGCCCGACGCCCGGGCTTTGCCCGGGCGGCCTCAG |
| | | TGAGCGAGCGAGCGCGCAGGATCCGAGCTTGTCGAGAAGTACTAG |
| | | AGGATCATAATCAGCCATACCACATTTGTAGAGGTTTTACTTGCTT |
| | | TAAAAAACCTCCCACACCTCCCCCTGAACCTGAAACATAAAATGA |
| | | ATGCAATTGTTGTTGTTAACTTGTTTATTGCAGCTTATAATGGTTAC |
| | | AAATAAAGCAATAGCATCACAAATTTCACAAATAAAGCATTTTTTT |
| | | CACTGCATTCTAGTTGTGGTTTGTCCAAACTCATCAATGTATCTTAT |
| | | CATGTCTGGATCTGATCACTGCTTGAGCCTAGGAGATCCGAACCAG |
| | | ATAAGTGAAATCTAGTTCCAAACTATTTTGTCATTTTTAATTTTCGT |
| | | ATTAGCTTACGACGCTACACCCAGTTCCCATCTATTTTGTCACTCTT |
| | | CCCTAAATAATCCTTAAAAACTCCATTTCCACCCCTCCCAGTTCCC |
| | | AACTATTTTGTCCGCCCACAGCGGGGCATTTTTCTTCCTGTTATGTT |
| | | TTTAATCAAACATCCTGCCAACTCCATGTGACAAACCGTCATCTTC |
| | | GGCTACTTTTTCTCTGTCACAGAATGAAAATTTTTCTGTCATCTCTT |
| | | CGTTATTAATGTTTGTAATTGACTGAATATCAACGCTTATTTGCAG |
| | | CCTGAATGGCGAATG |
| SEQ ID NO: 124 | Vector | GACGCGCCCTGTAGCGGCGCATTAAGCGCGGCGGGTGTGGTGGTT |
| | GTR | ACGCGCAGCGTGACCGCTACACTTGCCAGCGCCCTAGCGCCCGCTC |
| | Plasmid | CTTTCGCTTTCTTCCCTTCCTTTCTCGCCACGTTCGCCGGCTTTCCCC |
| | | GTCAAGCTCTAAATCGGGGGCTCCCTTTAGGGTTCCGATTTAGTGC |
| | | TTTACGGCACCTCGACCCCAAAAAACTTGATTAGGGTGATGGTTCA |
| | | CGTAGTGGGCCATCGCCCTGATAGACGGTTTTTCGCCCTTTGACGT |
| | | TGGAGTCCACGTTCTTTAATAGTGGACTCTTGTTCCAAACTGGAAC |
| | | AACACTCAACCCTATCTCGGTCTATTCTTTTGATTTATAAGGGATTT |
| | | TGCCGATTTCGGCCTATTGGTTAAAAAATGAGCTGATTTAACAAAA |
| | | ATTTAACGCGAATTTTAACAAAATATTAACGTTTACAATTTCAGGT |
| | | GGCACTTTTCGGGGAAATGTGCGCGGAACCCCTATTTGTTTATTTT |
| | | TCTAAATACATTCAAATATGTATCCGCTCATGAGACAATAACCCTG |
| | | ATAAATGCTTCAATAATATTGAAAAAGGAAGAGTATGAGTATTCA |
| | | ACATTTCCGTGTCGCCCTTATTCCCTTTTTTGCGGCATTTTGCCTTC |
| | | CTGTTTTTGCTCACCCAGAAACGCTGGTGAAAGTAAAAGATGCTGA |
| | | AGATCAGTTGGGTGCACGAGTGGGTTACATCGAACTGGATCTCAA |
| | | CAGCGGTAAGATCCTTGAGAGTTTTCGCCCCGAAGAACGTTTTCCA |
| | | ATGATGAGCACTTTTAAAGTTCTGCTATGTGGCGCGGTATTATCCC |
| | | GTATTGACGCCGGGCAAGAGCAACTCGGTCGCCGCATACACTATT |
| | | CTCAGAATGACTTGGTTGAGTACTCACCAGTCACAGAAAAGCATCT |
| | | TACGGATGGCATGACAGTAAGAGAATTATGCAGTGCTGCCATAAC |
| | | CATGAGTGATAACACTGCGGCCAACTTACTTCTGACAACGATCGG |
| | | AGGACCGAAGGAGCTAACCGCTTTTTTGCACAACATGGGGGATCA |
| | | TGTAACTCGCCTTGATCGTTGGGAACCGGAGCTGAATGAAGCCAT |
| | | ACCAAACGACGAGCGTGACACCACGATGCCTGTAGCAATGGCAAC |
| | | AACGTTGCGCAAACTATTAACTGGCGAACTACTTACTCTAGCTTCC |
| | | CGGCAACAATTAATAGACTGGATGGAGGCGGATAAAGTTGCAGGA |
| | | CCACTTCTGCGCTCGGCCCTTCCGGCTGGCTGGTTTATTGCTGATA |
| | | AATCTGGAGCCGGTGAGCGTGGGTCTCGCGGTATCATTGCAGCACT |
| | | GGGGCCAGATGGTAAGCCCTCCCGTATCGTAGTTATCTACACGACG |
| | | GGGAGTCAGGCAACTATGGATGAACGAAATAGACAGATCGCTGAG |
| | | ATAGGTGCCTCACTGATTAAGCATTGGTAACTGTCAGACCAAGTTT |
| | | ACTCATATATACTTTAGATTGATTTAAAACTTCATTTTTAATTTAAA |
| | | AGGATCTAGGTGAAGATCCTTTTTGATAATCTCATGACCAAAATCC |
| | | CTTAACGTGAGTTTTCGTTCCACTGAGCGTCAGACCCCGTAGAAAA |
| | | GATCAAAGGATCTTCTTGAGATCCTTTTTTTCTGCGCGTAATCTGCT |
| | | GCTTGCAAACAAAAAAACCACCGCTACCAGCGGTGGTTTGTTTGCC |
| | | GGATCAAGAGCTACCAACTCTTTTTCCGAAGGTAACTGGCTTCAGC |
| | | AGAGCGCAGATACCAAATACTGTCCTTCTAGTGTAGCCGTAGTTAG |
| | | GCCACCACTTCAAGAACTCTGTAGCACCGCCTACATACCTCGCTCT |
| | | GCTAATCCTGTTACCAGTGGCTGCTGCCAGTGGCGATAAGTCGTGT |
| | | CTTACCGGGTTGGACTCAAGACGATAGTTACCGGATAAGGCGCAG |
| | | CGGTCGGGCTGAACGGGGGGTTCGTGCACACAGCCCAGCTTGGAG |
| | | CGAACGACCTACACCGAACTGAGATACCTACAGCGTGAGCATTGA |
| | | GAAAGCGCCACGCTTCCCGAAGGGAGAAAGGCGGACAGGTATCCG |
| | | GTAAGCGGCAGGGTCGGAACAGGAGAGCGCACGAGGGAGCTTCC |
| | | AGGGGGAAACGCCTGGTATCTTTATAGTCCTGTCGGGTTTCGCCAC |
| | | CTCTGACTTGAGCGTCGATTTTTGTGATGCTCGTCAGGGGGGCGGA |
| | | GCCTATGGAAAAACGCCAGCAACGCGGCCTTTTTACGGTTCCTGGC |
| | | CTTTTGCTGGCCTTTTGCTCACATGTTCTTTCCTGCGTTATCCCCTG |
| | | ATTCTGTGGATAACCGTATTACCGCCTTTGAGTGAGCTGATACCGC |
| | | TCGCCGCAGCCGAACGACCGAGCGCAGCGAGTCAGTGAGCGAGGA |
| | | AGCGGAAGAGCGCCTGATGCGGTATTTTCTCCTTACGCATCTGTGC |
| | | GGTATTTCACACCGCAGACCAGCCGCGTAACCTGGCAAAATCGGT |
| | | TACGGTTGAGTAATAAATGGATGCCCTGCGTAAGCGGGTGTGGGC |
| | | GGACAATAAAGTCTTAAACTGAACAAAATAGATCTAAACTATGAC |
| | | AATAAAGTCTTAAACTAGACAGAATAGTTGTAAACTGAAATCAGT |
| | | CCAGTTATGCTGTGAAAAAGCATACTGGACTTTTGTTATGGCTAAA |
| | | GCAAACTCTTCATTTTCTGAAGTGCAAATTGCCCGTCGTATTAAAG |
| | | AGGGGCGTGGCCAAGGGCATGGTAAAGACTATATTCGCGGCGTTG |
| | | TGACAATTTACCGAACAACTCCGCGGCCGGGAAGCCGATCTCGGC |
| | | TTGAACGAATTGTTAGGTGGCGGTACTTGGGTCGATATCAAAGTGC |
| | | ATCACTTCTTCCCGTATGCCCAACTTTGTATAGAGAGCCACTGCGG |
| | | GATCGTCACCGTAATCTGCTTGCACGTAGATCACATAAGCACCAAG |
| | | CGCGTTGGCCTCATGCTTGAGGAGATTGATGAGCGCGGTGGCAAT |
| | | GCCCTGCCTCCGGTGCTCGCCGGAGACTGCGAGATCATAGATATA |
| | | GATCTCACTACGCGGCTGCTCAAACCTGGGCAGAACGTAAGCCGC |
| | | GAGAGCGCCAACAACCGCTTCTTGGTCGAAGGCAGCAAGCGCGAT |
| | | GAATGTCTTACTACGGAGCAAGTTCCCGAGGTAATCGGAGTCCGG |
| | | CTGATGTTGGGAGTAGGTGGCTACGTCTCCGAACTCACGACCGAA |
| | | AAGATCAAGAGCAGCCCGCATGGATTTGACTTGGTCAGGGCCGAG |
| | | CCTACATGTGCGAATGATGCCCATACTTGAGCCACCTAACTTTGTT |
| | | TTAGGGCGACTGCCTTGCTGTGTAACGTCATTGCTGCTGCGTAACA |
| | | TCGTTGCTGCTCCATAACATCAAACATCGACCCACGGCGTAACGCG |
| | | CTTGCTGCTTGGATGCCCGAGGCATAGACTGTACAAAAAAACAGT |
| | | CATAACAAGCCATGAAAACCGCCACTGCGCCGTTACCACCGCTGC |
| | | GTTCGGTCAAGGTTCTGGACCAGTTGCGTGAGCGCATACGCTACTT |
| | | GCATTACAGTTTACGAACCGAACAGGCTTATGTCAACTGGGTTCGT |
| | | GCCTTCATCCGTTTCCACGGTGTGCGTCACCCGGCAACCTTGGGCA |
| | | GCAGCGAAGTCGAGGCATTTCTGTCCTGGCTGGCGAACGAGCGCA |
| | | AGGTTTCGGTCTCCACGCATCGTCAGGCATTGGCGGCCTTGCTGTT |
| | | CTTCTACGGCAAGGTGCTGTGCACGGATCTGCCCTGGCTTCAGGAG |
| | | ATCGGAAGACCTCGGCCGTCGCGGCGCTTGCCGGTGGTGCTGACC |
| | | CCGGATGAAGTGGTTCGCATCCTCGGTTTTCTGGAAGGCGAGCATC |
| | | GTTTGTTCGCCCAGGACTCTAGCTATAGTTCTAGTGGTTGGCTACA |
| | | GCTTGCTGCGCGCTCGCTCGCTCACTGAGGCCGCCCGGGCAAAGCC |
| | | CGGGCGTCGGGCGACCTTTGGTCGCCCGGCCTCAGTGAGCGAGCG |
| | | AGCGCGCAGAGAGGGAGTGGCCAACTCCATCACTAGGGGTTCCTA |
| | | GGAAGCTGATCTGAATTCGGTACCCGTTACATAACTTACGGTAAAT |
| | | GGCCCGCCTGGCTGACCGCCCAACGACCCCCGCCCATTGACGTCA |
| | | ATAATGACGTATGTTCCCATAGTAACGCCAATAGGGACTTTCCATT |
| | | GACGTCAATGGGTGGAGTATTTACGGTAAACTGCCCACTTGGCAGT |
| | | ACATCAAGTGTATCATATGCCAAGTACGCCCCCTATTGACGTCAAT |
| | | GACGGTAAATGGCCCGCCTGGCATTATGCCCAGTACATGACCTTAT |
| | | GGGACTTTCCTACTTGGCAGTACATCTACGTATTAGTCATCGCTAT |
| | | TACCATGGCTCGAGGTGAGCCCCACGTTCTGCTTCACTCTCCCCAT |
| | | CTCCCCCCCCTCCCCACCCCCAATTTTGTATTTATTTATTTTTTAATT |
| | | ATTTTGTGCAGCGATGGGGGCGGGGGGGGGGGGGGGGCGCGCGCC |
| | | AGGCGGGGCGGGGCGGGGCGAGGGGCGGGGCGGGGCGAGGCGGA |
| | | GAGGTGCGGCGGCAGCCAATCAGAGCGGCGCGCTCCGAAAGTTTC |
| | | CTTTTATGGCGAGGCGGCGGCGGCGGCGGCCCTATAAAAAGCGAA |
| | | GCGCGCGGCGGGCGGGAGTCGCTGCGTTGCCTTCGCCCCGTGCCCC |
| | | GCTCCGCGCCGCCTCGCGCCGCCCGCCCCGGCTCTGACTGACCGCG |
| | | TTACTCCCACAGGTAAGTTTAGTCTTTTTGTCTTTTATTTCAGGTCC |
| | | CGGATCCGGTGGTGGTGCAAATCAAAGAACTGCTCCTCAGTGGAT |
| | | GTTGCCTTTACTTCTAGGCCTGCCGCCACCATGGGAATCCAAGGCG |
| | | GCTCTGTGCTGTTTGGCCTGCTGCTGGTTCTGGCCGTGTTTTGTCAC |
| | | TCTGGCCACAGCCTGCAGTGCTACAACTGCCCTAATCCTACCGCCG |
| | | ACTGCAAGACCGCCGTGAACTGCAGCAGCGATTTCGACGCCTGCC |
| | | TGATCACAAAGGCCGGACTGCAGGTCTACAACAAGTGCTGGAAGT |
| | | TCGAGCACTGCAACTTCAACGACGTGACCACCAGACTGAGAGAGA |
| | | ACGAGCTGACCTACTACTGCTGCAAGAAAGACCTGTGCAATTTCA |
| | | ATGAGCAGCTGGAAAACGGCGGCACCAGCCTGAGCGAAAAGACA |
| | | GTGCTGCTGCTCGTGACCCCTTTCCTGGCTGCTGCTTGGAGCCTGC |
| | | ATCCTTGAAGAACCAGGTGAGCCTGACCTGCCTGGTGAAGGGCTT |
| | | CTACCCCAGCGACATCGCCGTGGAGTGGGAGAGCAACGGCCAGCC |
| | | CGAGAACAACTACAAGACCACCCCCCCCGTGCTGGACAGCGACGG |
| | | CAGCTTCTTCCTGTACAGCAGACTGACCGTGGACAAGAGCAGATG |
| | | GCAGGAGGGCAACGTGTTCAGCTGCAGCGTGATGCACGAGGCCCT |
| | | GCACAACCACTACACCCAGAAGAGCCTGAGCCTGAGCCTGGGCAA |
| | | GGGCGGAGGCGGAAGCGGCGGAGGCGGATCTGGCGGAGGCGGAA |
| | | GCGGCGGAGGCGGCTCTGAGCACCCCAACGCGCGCAAATACAAAG |
| | | GAGCCAACAAGAAGGGCTTGTCCAAGGGCTGCTTCGGCCTCAAGC |
| | | TGGACCGAATCGGCTCCATGAGCGGCCTGGGATGTTAACTCGAGA |
| | | ATCAACCTCTGGATTACAAAATTTGTGAAAGATTGACTGGTATTCT |
| | | TAACTATGTTGCTCCTTTTACGCTATGTGGATACGCTGCTTTAATGC |
| | | CTTTGTATCATGCTATTGCTTCCCGTATGGCTTTCATTTTCTCCTCCT |
| | | TGTATAAATCCTGGTTGCTGTCTCTTTATGAGGAGTTGTGGCCCGTT |
| | | GTCAGGCAACGTGGCGTGGTGTGCACTGTGTTTGCTGACGCAACCC |
| | | CCACTGGTTGGGGCATTGCCACCACCTGTCAGCTCCTTTCCGGGAC |
| | | TTTCGCTTTCCCCCTCCCTATTGCCACGGCGGAACTCATCGCCGCCT |
| | | GCCTTGCCCGCTGCTGGACAGGGGCTCGGCTGTTGGGCACTGACA |
| | | ATTCCGTGGTGGTACCTTCGAAAATAAAATATCTTTATTTTCATTAC |
| | | ATCTGTGTGTTGGTTTTTTGTGTGGCATGCTGGGGAGAGATCAACC |
| | | CCACTCCCTCTCTGCGCGCTCGCTCGCTCACTGAGGCCGGGCGACC |
| | | AAAGGTCGCCCGACGCCCGGGCTTTGCCCGGGCGGCCTCAGTGAG |
| | | CGAGCGAGCGCGCAGGATCCGAGCTTGTCGAGAAGTACTAGAGGA |
| | | TCATAATCAGCCATACCACATTTGTAGAGGTTTTACTTGCTTTAAA |
| | | AAACCTCCCACACCTCCCCCTGAACCTGAAACATAAAATGAATGC |
| | | AATTGTTGTTGTTAACTTGTTTATTGCAGCTTATAATGGTTACAAAT |
| | | AAAGCAATAGCATCACAAATTTCACAAATAAAGCATTTTTTTCACT |
| | | GCATTCTAGTTGTGGTTTGTCCAAACTCATCAATGTATCTTATCATG |
| | | TCTGGATCTGATCACTGCTTGAGCCTAGGAGATCCGAACCAGATAA |
| | | GTGAAATCTAGTTCCAAACTATTTTGTCATTTTTAATTTTCGTATTA |
| | | GCTTACGACGCTACACCCAGTTCCCATCTATTTTGTCACTCTTCCCT |
| | | AAATAATCCTTAAAAACTCCATTTCCACCCCTCCCAGTTCCCAACT |
| | | ATTTTGTCCGCCCACAGCGGGGCATTTTTCTTCCTGTTATGTTTTTA |
| | | ATCAAACATCCTGCCAACTCCATGTGACAAACCGTCATCTTCGGCT |
| | | ACTTTTTCTCTGTCACAGAATGAAAATTTTTCTGTCATCTCTTCGTT |
| | | ATTAATGTTTGTAATTGACTGAATATCAACGCTTATTTGCAGCCTG |
| | | AATGGCGAATG |
| SEQ ID NO: 125 | Vector | GACGCGCCCTGTAGCGGCGCATTAAGCGCGGCGGGTGTGGTGGTT |
| | GAT | ACGCGCAGCGTGACCGCTACACTTGCCAGCGCCCTAGCGCCCGCTC |
| | Plasmid | CTTTCGCTTTCTTCCCTTCCTTTCTCGCCACGTTCGCCGGCTTTCCCC |
| | | GTCAAGCTCTAAATCGGGGGCTCCCTTTAGGGTTCCGATTTAGTGC |
| | | TTTACGGCACCTCGACCCCAAAAAACTTGATTAGGGTGATGGTTCA |
| | | CGTAGTGGGCCATCGCCCTGATAGACGGTTTTTCGCCCTTTGACGT |
| | | TGGAGTCCACGTTCTTTAATAGTGGACTCTTGTTCCAAACTGGAAC |
| | | AACACTCAACCCTATCTCGGTCTATTCTTTTGATTTATAAGGGATTT |
| | | TGCCGATTTCGGCCTATTGGTTAAAAAATGAGCTGATTTAACAAAA |
| | | ATTTAACGCGAATTTTAACAAAATATTAACGTTTACAATTTCAGGT |
| | | GGCACTTTTCGGGGAAATGTGCGCGGAACCCCTATTTGTTTATTTT |
| | | TCTAAATACATTCAAATATGTATCCGCTCATGAGACAATAACCCTG |
| | | ATAAATGCTTCAATAATATTGAAAAAGGAAGAGTATGAGTATTCA |
| | | ACATTTCCGTGTCGCCCTTATTCCCTTTTTTGCGGCATTTTGCCTTC |
| | | CTGTTTTTGCTCACCCAGAAACGCTGGTGAAAGTAAAAGATGCTGA |
| | | AGATCAGTTGGGTGCACGAGTGGGTTACATCGAACTGGATCTCAA |
| | | CAGCGGTAAGATCCTTGAGAGTTTTCGCCCCGAAGAACGTTTTCCA |
| | | ATGATGAGCACTTTTAAAGTTCTGCTATGTGGCGCGGTATTATCCC |
| | | GTATTGACGCCGGGCAAGAGCAACTCGGTCGCCGCATACACTATT |
| | | CTCAGAATGACTTGGTTGAGTACTCACCAGTCACAGAAAAGCATCT |
| | | TACGGATGGCATGACAGTAAGAGAATTATGCAGTGCTGCCATAAC |
| | | CATGAGTGATAACACTGCGGCCAACTTACTTCTGACAACGATCGG |
| | | AGGACCGAAGGAGCTAACCGCTTTTTTGCACAACATGGGGGATCA |
| | | TGTAACTCGCCTTGATCGTTGGGAACCGGAGCTGAATGAAGCCAT |
| | | ACCAAACGACGAGCGTGACACCACGATGCCTGTAGCAATGGCAAC |
| | | AACGTTGCGCAAACTATTAACTGGCGAACTACTTACTCTAGCTTCC |
| | | CGGCAACAATTAATAGACTGGATGGAGGCGGATAAAGTTGCAGGA |
| | | CCACTTCTGCGCTCGGCCCTTCCGGCTGGCTGGTTTATTGCTGATA |
| | | AATCTGGAGCCGGTGAGCGTGGGTCTCGCGGTATCATTGCAGCACT |
| | | GGGGCCAGATGGTAAGCCCTCCCGTATCGTAGTTATCTACACGACG |
| | | GGGAGTCAGGCAACTATGGATGAACGAAATAGACAGATCGCTGAG |
| | | ATAGGTGCCTCACTGATTAAGCATTGGTAACTGTCAGACCAAGTTT |
| | | ACTCATATATACTTTAGATTGATTTAAAACTTCATTTTTAATTTAAA |
| | | AGGATCTAGGTGAAGATCCTTTTTGATAATCTCATGACCAAAATCC |
| | | CTTAACGTGAGTTTTCGTTCCACTGAGCGTCAGACCCCGTAGAAAA |
| | | GATCAAAGGATCTTCTTGAGATCCTTTTTTTCTGCGCGTAATCTGCT |
| | | GCTTGCAAACAAAAAAACCACCGCTACCAGCGGTGGTTTGTTTGCC |
| | | GGATCAAGAGCTACCAACTCTTTTTCCGAAGGTAACTGGCTTCAGC |
| | | AGAGCGCAGATACCAAATACTGTCCTTCTAGTGTAGCCGTAGTTAG |
| | | GCCACCACTTCAAGAACTCTGTAGCACCGCCTACATACCTCGCTCT |
| | | GCTAATCCTGTTACCAGTGGCTGCTGCCAGTGGCGATAAGTCGTGT |
| | | CTTACCGGGTTGGACTCAAGACGATAGTTACCGGATAAGGCGCAG |
| | | CGGTCGGGCTGAACGGGGGGTTCGTGCACACAGCCCAGCTTGGAG |
| | | CGAACGACCTACACCGAACTGAGATACCTACAGCGTGAGCATTGA |
| | | GAAAGCGCCACGCTTCCCGAAGGGAGAAAGGCGGACAGGTATCCG |
| | | GTAAGCGGCAGGGTCGGAACAGGAGAGCGCACGAGGGAGCTTCC |
| | | AGGGGGAAACGCCTGGTATCTTTATAGTCCTGTCGGGTTTCGCCAC |
| | | CTCTGACTTGAGCGTCGATTTTTGTGATGCTCGTCAGGGGGGCGGA |
| | | GCCTATGGAAAAACGCCAGCAACGCGGCCTTTTTACGGTTCCTGGC |
| | | CTTTTGCTGGCCTTTTGCTCACATGTTCTTTCCTGCGTTATCCCCTG |
| | | ATTCTGTGGATAACCGTATTACCGCCTTTGAGTGAGCTGATACCGC |
| | | TCGCCGCAGCCGAACGACCGAGCGCAGCGAGTCAGTGAGCGAGGA |
| | | AGCGGAAGAGCGCCTGATGCGGTATTTTCTCCTTACGCATCTGTGC |
| | | GGTATTTCACACCGCAGACCAGCCGCGTAACCTGGCAAAATCGGT |
| | | TACGGTTGAGTAATAAATGGATGCCCTGCGTAAGCGGGTGTGGGC |
| | | GGACAATAAAGTCTTAAACTGAACAAAATAGATCTAAACTATGAC |
| | | AATAAAGTCTTAAACTAGACAGAATAGTTGTAAACTGAAATCAGT |
| | | CCAGTTATGCTGTGAAAAAGCATACTGGACTTTTGTTATGGCTAAA |
| | | GCAAACTCTTCATTTTCTGAAGTGCAAATTGCCCGTCGTATTAAAG |
| | | AGGGGCGTGGCCAAGGGCATGGTAAAGACTATATTCGCGGCGTTG |
| | | TGACAATTTACCGAACAACTCCGCGGCCGGGAAGCCGATCTCGGC |
| | | TTGAACGAATTGTTAGGTGGCGGTACTTGGGTCGATATCAAAGTGC |
| | | ATCACTTCTTCCCGTATGCCCAACTTTGTATAGAGAGCCACTGCGG |
| | | GATCGTCACCGTAATCTGCTTGCACGTAGATCACATAAGCACCAAG |
| | | CGCGTTGGCCTCATGCTTGAGGAGATTGATGAGCGCGGTGGCAAT |
| | | GCCCTGCCTCCGGTGCTCGCCGGAGACTGCGAGATCATAGATATA |
| | | GATCTCACTACGCGGCTGCTCAAACCTGGGCAGAACGTAAGCCGC |
| | | GAGAGCGCCAACAACCGCTTCTTGGTCGAAGGCAGCAAGCGCGAT |
| | | GAATGTCTTACTACGGAGCAAGTTCCCGAGGTAATCGGAGTCCGG |
| | | CTGATGTTGGGAGTAGGTGGCTACGTCTCCGAACTCACGACCGAA |
| | | AAGATCAAGAGCAGCCCGCATGGATTTGACTTGGTCAGGGCCGAG |
| | | CCTACATGTGCGAATGATGCCCATACTTGAGCCACCTAACTTTGTT |
| | | TTAGGGCGACTGCCTTGCTGTGTAACGTCATTGCTGCTGCGTAACA |
| | | TCGTTGCTGCTCCATAACATCAAACATCGACCCACGGCGTAACGCG |
| | | CTTGCTGCTTGGATGCCCGAGGCATAGACTGTACAAAAAAACAGT |
| | | CATAACAAGCCATGAAAACCGCCACTGCGCCGTTACCACCGCTGC |
| | | GTTCGGTCAAGGTTCTGGACCAGTTGCGTGAGCGCATACGCTACTT |
| | | GCATTACAGTTTACGAACCGAACAGGCTTATGTCAACTGGGTTCGT |
| | | GCCTTCATCCGTTTCCACGGTGTGCGTCACCCGGCAACCTTGGGCA |
| | | GCAGCGAAGTCGAGGCATTTCTGTCCTGGCTGGCGAACGAGCGCA |
| | | AGGTTTCGGTCTCCACGCATCGTCAGGCATTGGCGGCCTTGCTGTT |
| | | CTTCTACGGCAAGGTGCTGTGCACGGATCTGCCCTGGCTTCAGGAG |
| | | ATCGGAAGACCTCGGCCGTCGCGGCGCTTGCCGGTGGTGCTGACC |
| | | CCGGATGAAGTGGTTCGCATCCTCGGTTTTCTGGAAGGCGAGCATC |
| | | GTTTGTTCGCCCAGGACTCTAGCTATAGTTCTAGTGGTTGGCTACA |
| | | GCTTGCTGCGCGCTCGCTCGCTCACTGAGGCCGCCCGGGCAAAGCC |
| | | CGGGCGTCGGGCGACCTTTGGTCGCCCGGCCTCAGTGAGCGAGCG |
| | | AGCGCGCAGAGAGGGAGTGGCCAACTCCATCACTAGGGGTTCCTA |
| | | GGAAGCTGATCTGAATTCGGTACCCGTTACATAACTTACGGTAAAT |
| | | GGCCCGCCTGGCTGACCGCCCAACGACCCCCGCCCATTGACGTCA |
| | | ATAATGACGTATGTTCCCATAGTAACGCCAATAGGGACTTTCCATT |
| | | GACGTCAATGGGTGGAGTATTTACGGTAAACTGCCCACTTGGCAGT |
| | | ACATCAAGTGTATCATATGCCAAGTACGCCCCCTATTGACGTCAAT |
| | | GACGGTAAATGGCCCGCCTGGCATTATGCCCAGTACATGACCTTAT |
| | | GGGACTTTCCTACTTGGCAGTACATCTACGTATTAGTCATCGCTAT |
| | | TACCATGGCTCGAGGTGAGCCCCACGTTCTGCTTCACTCTCCCCAT |
| | | CTCCCCCCCCTCCCCACCCCCAATTTTGTATTTATTTATTTTTTAATT |
| | | ATTTTGTGCAGCGATGGGGGCGGGGGGGGGGGGGGGGCGCGCGCC |
| | | AGGCGGGGGGGGCGGGGCGAGGGGCGGGGCGGGGCGAGGCGGA |
| | | GAGGTGCGGCGGCAGCCAATCAGAGCGGCGCGCTCCGAAAGTTTC |
| | | CTTTTATGGCGAGGCGGCGGCGGCGGCGGCCCTATAAAAAGCGAA |
| | | GCGCGCGGCGGGCGGGAGTCGCTGCGTTGCCTTCGCCCCGTGCCCC |
| | | GCTCCGCGCCGCCTCGCGCCGCCCGCCCCGGCTCTGACTGACCGCG |
| | | TTACTCCCACAGGTAAGTTTAGTCTTTTTGTCTTTTATTTCAGGTCC |
| | | CGGATCCGGTGGTGGTGCAAATCAAAGAACTGCTCCTCAGTGGAT |
| | | GTTGCCTTTACTTCTAGGCCTGCCGCCACCATGGGCATCCAGGGCG |
| | | GCAGCGTGCTGTTTGGACTGCTGCTGGTCCTCGCCGTGTTCTGCCA |
| | | CAGCGGCCATTCTCTGCAATGTTATAACTGTCCTAACCCAACAGCT |
| | | GATTGCAAAACAGCCGTGAACTGCAGCTCTGATTTCGACGCCTGCC |
| | | TGATCACCAAGGCCGGCCTGCAGGTGTACAACAAGTGCTGGAAGT |
| | | TCGAGCACTGTAATTTTAACGACGTGACCACCAGACTGCGGGAAA |
| | | ATGAGCTGACCTACTACTGCTGCAAAAAGGACCTGTGCAACTTCA |
| | | ACGAGCAGCTGGAAAACGGCGGAACCAGCCTGAGCGAGAAGACC |
| | | GTGCTGCTGCTGGTGACACCTTTCCTGGCCGCCGCTTGGTCCCTGC |
| | | ACCCCTAAAGAACCAGGTGAGCCTGACCTGCCTGGTGAAGGGCTT |
| | | CTACCCCAGCGACATCGCCGTGGAGTGGGAGAGCAACGGCCAGCC |
| | | CGAGAACAACTACAAGACCACCCCCCCCGTGCTGGACAGCGACGG |
| | | CAGCTTCTTCCTGTACAGCAGACTGACCGTGGACAAGAGCAGATG |
| | | GCAGGAGGGCAACGTGTTCAGCTGCAGCGTGATGCACGAGGCCCT |
| | | GCACAACCACTACACCCAGAAGAGCCTGAGCCTGAGCCTGGGCAA |
| | | GGGCGGAGGCGGAAGCGGCGGAGGCGGATCTGGCGGAGGCGGAA |
| | | GCGGCGGAGGCGGCTCTGAGCACCCCAACGCGCGCAAATACAAAG |
| | | GAGCCAACAAGAAGGGCTTGTCCAAGGGCTGCTTCGGCCTCAAGC |
| | | TGGACCGAATCGGCTCCATGAGCGGCCTGGGATGTTAACTCGAGA |
| | | ATCAACCTCTGGATTACAAAATTTGTGAAAGATTGACTGGTATTCT |
| | | TAACTATGTTGCTCCTTTTACGCTATGTGGATACGCTGCTTTAATGC |
| | | CTTTGTATCATGCTATTGCTTCCCGTATGGCTTTCATTTTCTCCTCCT |
| | | TGTATAAATCCTGGTTGCTGTCTCTTTATGAGGAGTTGTGGCCCGTT |
| | | GTCAGGCAACGTGGCGTGGTGTGCACTGTGTTTGCTGACGCAACCC |
| | | CCACTGGTTGGGGCATTGCCACCACCTGTCAGCTCCTTTCCGGGAC |
| | | TTTCGCTTTCCCCCTCCCTATTGCCACGGCGGAACTCATCGCCGCCT |
| | | GCCTTGCCCGCTGCTGGACAGGGGCTCGGCTGTTGGGCACTGACA |
| | | ATTCCGTGGTGGTACCTTCGAAAATAAAATATCTTTATTTTCATTAC |
| | | ATCTGTGTGTTGGTTTTTTGTGTGGCATGCTGGGGAGAGATCAACC |
| | | CCACTCCCTCTCTGCGCGCTCGCTCGCTCACTGAGGCCGGGCGACC |
| | | AAAGGTCGCCCGACGCCCGGGCTTTGCCCGGGCGGCCTCAGTGAG |
| | | CGAGCGAGCGCGCAGGATCCGAGCTTGTCGAGAAGTACTAGAGGA |
| | | TCATAATCAGCCATACCACATTTGTAGAGGTTTTACTTGCTTTAAA |
| | | AAACCTCCCACACCTCCCCCTGAACCTGAAACATAAAATGAATGC |
| | | AATTGTTGTTGTTAACTTGTTTATTGCAGCTTATAATGGTTACAAAT |
| | | AAAGCAATAGCATCACAAATTTCACAAATAAAGCATTTTTTTCACT |
| | | GCATTCTAGTTGTGGTTTGTCCAAACTCATCAATGTATCTTATCATG |
| | | TCTGGATCTGATCACTGCTTGAGCCTAGGAGATCCGAACCAGATAA |
| | | GTGAAATCTAGTTCCAAACTATTTTGTCATTTTTAATTTTCGTATTA |
| | | GCTTACGACGCTACACCCAGTTCCCATCTATTTTGTCACTCTTCCCT |
| | | AAATAATCCTTAAAAACTCCATTTCCACCCCTCCCAGTTCCCAACT |
| | | ATTTTGTCCGCCCACAGCGGGGCATTTTTCTTCCTGTTATGTTTTTA |
| | | ATCAAACATCCTGCCAACTCCATGTGACAAACCGTCATCTTCGGCT |
| | | ACTTTTTCTCTGTCACAGAATGAAAATTTTTCTGTCATCTCTTCGTT |
| | | ATTAATGTTTGTAATTGACTGAATATCAACGCTTATTTGCAGCCTG |
| | | AATGGCGAATG |
| SEQ ID NO: 126 | Vector | GACGCGCCCTGTAGCGGCGCATTAAGCGCGGCGGGTGTGGTGGTT |
| | GCK | ACGCGCAGCGTGACCGCTACACTTGCCAGCGCCCTAGCGCCCGCTC |
| | Plasmid | CTTTCGCTTTCTTCCCTTCCTTTCTCGCCACGTTCGCCGGCTTTCCCC |
| | | GTCAAGCTCTAAATCGGGGGCTCCCTTTAGGGTTCCGATTTAGTGC |
| | | TTTACGGCACCTCGACCCCAAAAAACTTGATTAGGGTGATGGTTCA |
| | | CGTAGTGGGCCATCGCCCTGATAGACGGTTTTTCGCCCTTTGACGT |
| | | TGGAGTCCACGTTCTTTAATAGTGGACTCTTGTTCCAAACTGGAAC |
| | | AACACTCAACCCTATCTCGGTCTATTCTTTTGATTTATAAGGGATTT |
| | | TGCCGATTTCGGCCTATTGGTTAAAAAATGAGCTGATTTAACAAAA |
| | | ATTTAACGCGAATTTTAACAAAATATTAACGTTTACAATTTCAGGT |
| | | GGCACTTTTCGGGGAAATGTGCGCGGAACCCCTATTTGTTTATTTT |
| | | TCTAAATACATTCAAATATGTATCCGCTCATGAGACAATAACCCTG |
| | | ATAAATGCTTCAATAATATTGAAAAAGGAAGAGTATGAGTATTCA |
| | | ACATTTCCGTGTCGCCCTTATTCCCTTTTTTGCGGCATTTTGCCTTC |
| | | CTGTTTTTGCTCACCCAGAAACGCTGGTGAAAGTAAAAGATGCTGA |
| | | AGATCAGTTGGGTGCACGAGTGGGTTACATCGAACTGGATCTCAA |
| | | CAGCGGTAAGATCCTTGAGAGTTTTCGCCCCGAAGAACGTTTTCCA |
| | | ATGATGAGCACTTTTAAAGTTCTGCTATGTGGCGCGGTATTATCCC |
| | | GTATTGACGCCGGGCAAGAGCAACTCGGTCGCCGCATACACTATT |
| | | CTCAGAATGACTTGGTTGAGTACTCACCAGTCACAGAAAAGCATCT |
| | | TACGGATGGCATGACAGTAAGAGAATTATGCAGTGCTGCCATAAC |
| | | CATGAGTGATAACACTGCGGCCAACTTACTTCTGACAACGATCGG |
| | | AGGACCGAAGGAGCTAACCGCTTTTTTGCACAACATGGGGGATCA |
| | | TGTAACTCGCCTTGATCGTTGGGAACCGGAGCTGAATGAAGCCAT |
| | | ACCAAACGACGAGCGTGACACCACGATGCCTGTAGCAATGGCAAC |
| | | AACGTTGCGCAAACTATTAACTGGCGAACTACTTACTCTAGCTTCC |
| | | CGGCAACAATTAATAGACTGGATGGAGGCGGATAAAGTTGCAGGA |
| | | CCACTTCTGCGCTCGGCCCTTCCGGCTGGCTGGTTTATTGCTGATA |
| | | AATCTGGAGCCGGTGAGCGTGGGTCTCGCGGTATCATTGCAGCACT |
| | | GGGGCCAGATGGTAAGCCCTCCCGTATCGTAGTTATCTACACGACG |
| | | GGGAGTCAGGCAACTATGGATGAACGAAATAGACAGATCGCTGAG |
| | | ATAGGTGCCTCACTGATTAAGCATTGGTAACTGTCAGACCAAGTTT |
| | | ACTCATATATACTTTAGATTGATTTAAAACTTCATTTTTAATTTAAA |
| | | AGGATCTAGGTGAAGATCCTTTTTGATAATCTCATGACCAAAATCC |
| | | CTTAACGTGAGTTTTCGTTCCACTGAGCGTCAGACCCCGTAGAAAA |
| | | GATCAAAGGATCTTCTTGAGATCCTTTTTTTCTGCGCGTAATCTGCT |
| | | GCTTGCAAACAAAAAAACCACCGCTACCAGCGGTGGTTTGTTTGCC |
| | | GGATCAAGAGCTACCAACTCTTTTTCCGAAGGTAACTGGCTTCAGC |
| | | AGAGCGCAGATACCAAATACTGTCCTTCTAGTGTAGCCGTAGTTAG |
| | | GCCACCACTTCAAGAACTCTGTAGCACCGCCTACATACCTCGCTCT |
| | | GCTAATCCTGTTACCAGTGGCTGCTGCCAGTGGCGATAAGTCGTGT |
| | | CTTACCGGGTTGGACTCAAGACGATAGTTACCGGATAAGGCGCAG |
| | | CGGTCGGGCTGAACGGGGGGTTCGTGCACACAGCCCAGCTTGGAG |
| | | CGAACGACCTACACCGAACTGAGATACCTACAGCGTGAGCATTGA |
| | | GAAAGCGCCACGCTTCCCGAAGGGAGAAAGGCGGACAGGTATCCG |
| | | GTAAGCGGCAGGGTCGGAACAGGAGAGCGCACGAGGGAGCTTCC |
| | | AGGGGGAAACGCCTGGTATCTTTATAGTCCTGTCGGGTTTCGCCAC |
| | | CTCTGACTTGAGCGTCGATTTTTGTGATGCTCGTCAGGGGGGCGGA |
| | | GCCTATGGAAAAACGCCAGCAACGCGGCCTTTTTACGGTTCCTGGC |
| | | CTTTTGCTGGCCTTTTGCTCACATGTTCTTTCCTGCGTTATCCCCTG |
| | | ATTCTGTGGATAACCGTATTACCGCCTTTGAGTGAGCTGATACCGC |
| | | TCGCCGCAGCCGAACGACCGAGCGCAGCGAGTCAGTGAGCGAGGA |
| | | AGCGGAAGAGCGCCTGATGCGGTATTTTCTCCTTACGCATCTGTGC |
| | | GGTATTTCACACCGCAGACCAGCCGCGTAACCTGGCAAAATCGGT |
| | | TACGGTTGAGTAATAAATGGATGCCCTGCGTAAGCGGGTGTGGGC |
| | | GGACAATAAAGTCTTAAACTGAACAAAATAGATCTAAACTATGAC |
| | | AATAAAGTCTTAAACTAGACAGAATAGTTGTAAACTGAAATCAGT |
| | | CCAGTTATGCTGTGAAAAAGCATACTGGACTTTTGTTATGGCTAAA |
| | | GCAAACTCTTCATTTTCTGAAGTGCAAATTGCCCGTCGTATTAAAG |
| | | AGGGGCGTGGCCAAGGGCATGGTAAAGACTATATTCGCGGCGTTG |
| | | TGACAATTTACCGAACAACTCCGCGGCCGGGAAGCCGATCTCGGC |
| | | TTGAACGAATTGTTAGGTGGCGGTACTTGGGTCGATATCAAAGTGC |
| | | ATCACTTCTTCCCGTATGCCCAACTTTGTATAGAGAGCCACTGCGG |
| | | GATCGTCACCGTAATCTGCTTGCACGTAGATCACATAAGCACCAAG |
| | | CGCGTTGGCCTCATGCTTGAGGAGATTGATGAGCGCGGTGGCAAT |
| | | GCCCTGCCTCCGGTGCTCGCCGGAGACTGCGAGATCATAGATATA |
| | | GATCTCACTACGCGGCTGCTCAAACCTGGGCAGAACGTAAGCCGC |
| | | GAGAGCGCCAACAACCGCTTCTTGGTCGAAGGCAGCAAGCGCGAT |
| | | GAATGTCTTACTACGGAGCAAGTTCCCGAGGTAATCGGAGTCCGG |
| | | CTGATGTTGGGAGTAGGTGGCTACGTCTCCGAACTCACGACCGAA |
| | | AAGATCAAGAGCAGCCCGCATGGATTTGACTTGGTCAGGGCCGAG |
| | | CCTACATGTGCGAATGATGCCCATACTTGAGCCACCTAACTTTGTT |
| | | TTAGGGCGACTGCCTTGCTGTGTAACGTCATTGCTGCTGCGTAACA |
| | | TCGTTGCTGCTCCATAACATCAAACATCGACCCACGGCGTAACGCG |
| | | CTTGCTGCTTGGATGCCCGAGGCATAGACTGTACAAAAAAACAGT |
| | | CATAACAAGCCATGAAAACCGCCACTGCGCCGTTACCACCGCTGC |
| | | GTTCGGTCAAGGTTCTGGACCAGTTGCGTGAGCGCATACGCTACTT |
| | | GCATTACAGTTTACGAACCGAACAGGCTTATGTCAACTGGGTTCGT |
| | | GCCTTCATCCGTTTCCACGGTGTGCGTCACCCGGCAACCTTGGGCA |
| | | GCAGCGAAGTCGAGGCATTTCTGTCCTGGCTGGCGAACGAGCGCA |
| | | AGGTTTCGGTCTCCACGCATCGTCAGGCATTGGCGGCCTTGCTGTT |
| | | CTTCTACGGCAAGGTGCTGTGCACGGATCTGCCCTGGCTTCAGGAG |
| | | ATCGGAAGACCTCGGCCGTCGCGGCGCTTGCCGGTGGTGCTGACC |
| | | CCGGATGAAGTGGTTCGCATCCTCGGTTTTCTGGAAGGCGAGCATC |
| | | GTTTGTTCGCCCAGGACTCTAGCTATAGTTCTAGTGGTTGGCTACA |
| | | GCTTGCTGCGCGCTCGCTCGCTCACTGAGGCCGCCCGGGCAAAGCC |
| | | CGGGCGTCGGGCGACCTTTGGTCGCCCGGCCTCAGTGAGCGAGCG |
| | | AGCGCGCAGAGAGGGAGTGGCCAACTCCATCACTAGGGGTTCCTA |
| | | GGAAGCTGATCTGAATTCGGTACCCGTTACATAACTTACGGTAAAT |
| | | GGCCCGCCTGGCTGACCGCCCAACGACCCCCGCCCATTGACGTCA |
| | | ATAATGACGTATGTTCCCATAGTAACGCCAATAGGGACTTTCCATT |
| | | GACGTCAATGGGTGGAGTATTTACGGTAAACTGCCCACTTGGCAGT |
| | | ACATCAAGTGTATCATATGCCAAGTACGCCCCCTATTGACGTCAAT |
| | | GACGGTAAATGGCCCGCCTGGCATTATGCCCAGTACATGACCTTAT |
| | | GGGACTTTCCTACTTGGCAGTACATCTACGTATTAGTCATCGCTAT |
| | | TACCATGGCTCGAGGTGAGCCCCACGTTCTGCTTCACTCTCCCCAT |
| | | CTCCCCCCCCTCCCCACCCCCAATTTTGTATTTATTTATTTTTTAATT |
| | | ATTTTGTGCAGCGATGGGGGCGGGGGGGGGGGGGGGGCGCGCGCC |
| | | AGGCGGGGCGGGGCGGGGCGAGGGGCGGGGCGGGGCGAGGCGGA |
| | | GAGGTGCGGCGGCAGCCAATCAGAGCGGCGCGCTCCGAAAGTTTC |
| | | CTTTTATGGCGAGGCGGCGGCGGCGGCGGCCCTATAAAAAGCGAA |
| | | GCGCGCGGCGGGCGGGAGTCGCTGCGTTGCCTTCGCCCCGTGCCCC |
| | | GCTCCGCGCCGCCTCGCGCCGCCCGCCCCGGCTCTGACTGACCGCG |
| | | TTACTCCCACAGGTAAGTTTAGTCTTTTTGTCTTTTATTTCAGGTCC |
| | | CGGATCCGGTGGTGGTGCAAATCAAAGAACTGCTCCTCAGTGGAT |
| | | GTTGCCTTTACTTCTAGGCCTGCCGCCACCATGGGCATCCAGGGCG |
| | | GCAGCGTGCTGTTTGGACTGCTGCTGGTCCTCGCCGTGTTCTGCCA |
| | | CAGCGGCCATTCTCTGCAATGTTATAACTGTCCTAACCCAACAGCT |
| | | GATTGCAAAACAGCCGTGAACTGCAGCTCTGATTTCGACGCCTGCC |
| | | TGATCACCAAGGCCGGCCTGCAGGTGTACAACAAGTGCTGGAAGT |
| | | TCGAGCACTGTAATTTTAACGACGTGACCACCAGACTGCGGGAAA |
| | | ATGAGCTGACCTACTACTGCTGCAAAAAGGACCTGTGCAACTTCA |
| | | ACGAGCAGCTGGAAAACGGCGGAACCAGCCTGAGCGAGAAGACC |
| | | GTGCTGCTGCTGGTGACACCTTTCCTGGCCGCCGCTTGGTCCCTGC |
| | | ACCCCTAAAGAACCAGGTGAGCCTGACCTGCCTGGTGAAGGGCTT |
| | | CTACCCCAGCGACATCGCCGTGGAGTGGGAGAGCAACGGCCAGCC |
| | | CGAGAACAACTACAAGACCACCCCCCCCGTGCTGGACAGCGACGG |
| | | CAGCTTCTTCCTGTACAGCAGACTGACCGTGGACAAGAGCAGATG |
| | | GCAGGAGGGCAACGTGTTCAGCTGCAGCGTGATGCACGAGGCCCT |
| | | GCACAACCACTACACCCAGAAGAGCCTGAGCCTGAGCCTGGGCAA |
| | | GGGCGGAGGCGGAAGCGGCGGAGGCGGATCTGGCGGAGGCGGAA |
| | | GCGGCGGAGGCGGCTCTGAGCACCCCAACGCGCGCAAATACAAAG |
| | | GAGCCAACAAGAAGGGCTTGTCCAAGGGCTGCTTCGGCCTCAAGC |
| | | TGGACCGAATCGGCTCCATGAGCGGCCTGGGATGTTAACTCGAGA |
| | | ATCAACCTCTGGATTACAAAATTTGTGAAAGATTGACTGGTATTCT |
| | | TAACTATGTTGCTCCTTTTACGCTATGTGGATACGCTGCTTTAATGC |
| | | CTTTGTATCATGCTATTGCTTCCCGTATGGCTTTCATTTTCTCCTCCT |
| | | TGTATAAATCCTGGTTGCTGTCTCTTTATGAGGAGTTGTGGCCCGTT |
| | | GTCAGGCAACGTGGCGTGGTGTGCACTGTGTTTGCTGACGCAACCC |
| | | CCACTGGTTGGGGCATTGCCACCACCTGTCAGCTCCTTTCCGGGAC |
| | | TTTCGCTTTCCCCCTCCCTATTGCCACGGCGGAACTCATCGCCGCCT |
| | | GCCTTGCCCGCTGCTGGACAGGGGCTCGGCTGTTGGGCACTGACA |
| | | ATTCCGTGGTGGTACCTTCGAAAATAAAATATCTTTATTTTCATTAC |
| | | ATCTGTGTGTTGGTTTTTTGTGTGGCATGCTGGGGAGAGATCAACC |
| | | CCACTCCCTCTCTGCGCGCTCGCTCGCTCACTGAGGCCGGGCGACC |
| | | AAAGGTCGCCCGACGCCCGGGCTTTGCCCGGGCGGCCTCAGTGAG |
| | | CGAGCGAGCGCGCAGGATCCGAGCTTGTCGAGAAGTACTAGAGGA |
| | | TCATAATCAGCCATACCACATTTGTAGAGGTTTTACTTGCTTTAAA |
| | | AAACCTCCCACACCTCCCCCTGAACCTGAAACATAAAATGAATGC |
| | | AATTGTTGTTGTTAACTTGTTTATTGCAGCTTATAATGGTTACAAAT |
| | | AAAGCAATAGCATCACAAATTTCACAAATAAAGCATTTTTTTCACT |
| | | GCATTCTAGTTGTGGTTTGTCCAAACTCATCAATGTATCTTATCATG |
| | | TCTGGATCTGATCACTGCTTGAGCCTAGGAGATCCGAACCAGATAA |
| | | GTGAAATCTAGTTCCAAACTATTTTGTCATTTTTAATTTTCGTATTA |
| | | GCTTACGACGCTACACCCAGTTCCCATCTATTTTGTCACTCTTCCCT |
| | | AAATAATCCTTAAAAACTCCATTTCCACCCCTCCCAGTTCCCAACT |
| | | ATTTTGTCCGCCCACAGCGGGGCATTTTTCTTCCTGTTATGTTTTTA |
| | | ATCAAACATCCTGCCAACTCCATGTGACAAACCGTCATCTTCGGCT |
| | | ACTTTTTCTCTGTCACAGAATGAAAATTTTTCTGTCATCTCTTCGTT |
| | | ATTAATGTTTGTAATTGACTGAATATCAACGCTTATTTGCAGCCTG |
| | | AATGGCGAATG |
| SEQ ID NO: 127 | Vector | CATTCGCCATTCAGGCTGCAAATAAGCGTTGATATTCAGTCAATTA |
| | GCM | CAAACATTAATAACGAAGAGATGACAGAAAAATTTTCATTCTGTG |
| | Plasmid | ACAGAGAAAAAGTAGCCGAAGATGACGGTTTGTCACATGGAGTTG |
| | | GCAGGATGTTTGATTAAAAACATAACAGGAAGAAAAATGCCCCGC |
| | | TGTGGGCGGACAAAATAGTTGGGAACTGGGAGGGGTGGAAATGG |
| | | AGTTTTTAAGGATTATTTAGGGAAGAGTGACAAAATAGATGGGAA |
| | | CTGGGTGTAGCGTCGTAAGCTAATACGAAAATTAAAAATGACAAA |
| | | ATAGTTTGGAACTAGATTTCACTTATCTGGTTCGGATCTCCTAGGG |
| | | GTACCGGGCCCCCCCTCGAGGTCGACGGTATCGATGGATCCGATAT |
| | | CGAATTCAAGCGCGCTCGCTCGCTCACTGAGGCCGCCCGGGCAAA |
| | | GCCCGGGCGTCGGGCGACCTTTGGTCGCCCGGCCTCAGTGAGCGA |
| | | GCGAGCGCGCAGAGAGGGAGTGGCCAACTCCATCACTAGGGGTTC |
| | | CTTGTAGTTAATGATTAACCCGCCATGCTACTTATCTACGTACTCTG |
| | | GAGACGACTTACGGTAAATGGCCCGCCTGGCTGACCGCCCAACGA |
| | | CCCCCGCCCATTGACGTCAATAATGACGTATGTTCCCATAGTAACG |
| | | CCAATAGGGACTTTCCATTGACGTCAATGGGTGGAGTATTTACGGT |
| | | AAACTGCCCACTTGGCAGTACATCAAGTGTATCATATGCCAAGTCC |
| | | GCCCCCTATTGACGTCAATGACGGTAAATGGCCCGCCTGGCATTAT |
| | | GCCCAGTACATGACCTTACGGGACTTTCCTACTTGGCAGTACATCT |
| | | ACGTATTAGTCATCGCTATTACCATGGTCGAGGTGAGCCCCACGTT |
| | | CTGCTTCACTCTCCCCATCTCCCCCCCCTCCCCACCCCCAATTTTGT |
| | | ATTTATTTATTTTTTAATTATTTTGTGCAGCGATGGGGGGGGGGGG |
| | | GGGGGGGGGGGGCGCGCCAGGCGGGGCGGGGCGGGGCGAGGGGC |
| | | GGGGCGGGGCGAGGCGGAGAGGTGCGGCGGCAGCCAATCAGAGC |
| | | GGCGCGCTCCGAAAGTTTCCTTTTATGGCGAGGCGGCGGCGGCGG |
| | | CGGCCCTATAAAAAGCGAAGCGCGCGGCGGGCGGGAGTCGCTGCG |
| | | ACGCTGCCTTCGCCCCGTGCCCCGCTCCGCCGCCGCCTCGCGCCGC |
| | | CCGCCCCGGCTCTGACTGACCGCGTTACTCCCACAGAGATCTCGTT |
| | | TAGTGAACCGTCAGATCCTCACTCTCTTCCGCATCGCTGTCTGCGA |
| | | GGGCCAGCTGTTGGGCTCGCGGTTGAGGACAAACTCTTCGCGGTCT |
| | | TTCCAGTACTCTTGGATCGGAAACCCGTCGGCCTCCGAACGGTACT |
| | | CCGCCACCGAGGGACCTGAGCGAGTCCGCATCGACCGGATCGGAA |
| | | AACCTCTCGAGAAAGGCGTCTAACCAGTCACAGTCGCAAGGTAGG |
| | | CTGAGCACCGTGGCGGGCGGCAGCGGGTGGCGGTCGGGGTTGTTT |
| | | CTGGCGGAGGTGCTGCTGATGATGTAATTAAAGTAGGCGGTCTTG |
| | | AGACGGCGGATGGTCGAGGTGAGGTGTGGCAGGCTTGAGATCCAG |
| | | CTGTTGGGGTGAGTACTCCCTCTCAAAAGCGGGCATTACTTCTGCG |
| | | CTAAGATTGTCAGTTTCCAAAAACGAGGAGGATTTGATATTCACCT |
| | | GGCCCGATCTGGCCATACACTTGAGTGACAATGACATCCACTTTGC |
| | | CTTTCTCTCCACAGGTGTCCACTCCCAGGTCCAAGTTTAAACGCCG |
| | | CCACCATGGAGTTCGGCCTGAGCTGGCTGTTCCTGGTGGCCATCCT |
| | | TAAGGGCGTGCAGTGCGAGGTGCAGCTGCAGTGCTACAACTGTCC |
| | | TAACCCAACTGCTGACTGCAAAACAGCCGTCAATTGTTCATCTGAT |
| | | TTTGATGCGTGTCTCATTACCAAAGCTGGGTTACAAGTGTATAACA |
| | | AGTGTTGGAAGTTTGAGCATTGCAATTTCAACGACGTCACAACCCG |
| | | CTTGAGGGAAAATGAGCTAACGTACTACTGCTGCAAGAAGGACCT |
| | | GTGTAACTTTAACGAACAGCTTGAAAATTAACTCGAGAATCAACCT |
| | | CTGGATTACAAAATTTGTGAAAGATTGACTGGTATTCTTAACTATG |
| | | TTGCTCCTTTTACGCTATGTGGATACGCTGCTTTAATGCCTTTGTAT |
| | | CATGCTATTGCTTCCCGTATGGCTTTCATTTTCTCCTCCTTGTATAA |
| | | ATCCTGGTTGCTGTCTCTTTATGAGGAGTTGTGGCCCGTTGTCAGG |
| | | CAACGTGGCGTGGTGTGCACTGTGTTTGCTGACGCAACCCCCACTG |
| | | GTTGGGGCATTGCCACCACCTGTCAGCTCCTTTCCGGGACTTTCGC |
| | | TTTCCCCCTCCCTATTGCCACGGCGGAACTCATCGCCGCCTGCCTT |
| | | GCCCGCTGCTGGACAGGGGCTCGGCTGTTGGGCACTGACAATTCC |
| | | GTGGTCTGTTCTCATCACATCATATCAAGGTTATATACCATCAATA |
| | | TTGCCACAGATGTTACTTAGCCTTTTAATATTTCTCTAATTTAGTGT |
| | | ATATGCAATGATAGTTCTCTGATTTCTGAGATTGAGTTTCTCATGTG |
| | | TAATGATTATTTAGAGTTTCTCTTTCATCTGTTCAAATTTTTGTCTA |
| | | GTTTTATTTTTTACTGATTTGTAAGACTTCTTTTTATAATCTGCATAT |
| | | TACAATTCTCTTTACTGGGGTGTTGCAAATATTTTCTGTCATTCTAT |
| | | GGCCTGACTTTTCTTAATGGTTTTTTAATTTTAAAAATAAGTCTTAA |
| | | TATTCATGCAATCTAATTAACAATCTTTTCTTTGTGGTTAGGACTTT |
| | | GAGTCATAAGAAATTTTTCTCTACACTGAAGTCATGATGGCATGCT |
| | | TCTATATTATTTTCTAAAAGATTTAAAGTTTTGCCTTCTCCATTTAG |
| | | ACTTATAATTCACTGGAATTTTTTTGTGTGTATGGTATGACATATGG |
| | | GTTCCCTTTTATTTTTTACATATAAATATATTTCCCTGTTTTTCTAAA |
| | | AAAGAAAAAGATCATCATTTTCCCATTGTAAAATGCCATATTTTTT |
| | | TCATAGGTCACTTACATATATCAATGGGTCTGTTTCTGAGCTCTACT |
| | | CTATTTTATCAGCCTCACTGTCTATCCCCACACATCTCATGCTTTGC |
| | | TCTAAATCTTGATATTTAGTGGAACATTCTTTCCCATTTTGTTCTAC |
| | | AAGAATATTTTTGTTATTGTCTTTGGGCTTTCTATATACATTTTGAA |
| | | ATGAGGTTGACAAGTTACCTAGGAAAACTGTCTTCCTGCCCGGGTG |
| | | GCATCCCTGTGACCCCTCCCCAGTGCCTCTCCTGGCCCTGGAAGTT |
| | | GCCACTCCAGTGCCCACCAGCCTTGTCCTAATAAAATTAAGTTGCA |
| | | TCATTTTGTCTGACTAGGTGTCCTTCTATAATATTATGGGGTGGAG |
| | | GGGGGTGGTATGGAGCAAGGGGCCCAAGTTGGGAAGAAACCTGTA |
| | | GGGCCTGCGTACGTAGATAAGTAGCATGGCGGGTTAATCATTAAC |
| | | TACAAGGAACCCCTAGTGATGGAGTTGGCCACTCCCTCTCTGCGCG |
| | | CTCGCTCGCTCACTGAGGCCGGGCGACCAAAGGTCGCCCGACGCC |
| | | CGGGCTTTGCCCGGGCGGCCTCAGTGAGCGAGCGAGCGCGCAGCT |
| | | GCTGGTACCGGGCCCCCCCTCGAGGTCGACGGTATCGATGGATCC |
| | | GATATCGAATTCAACCTGCAGGCATGCTCTGAGACAATAACCCTG |
| | | ATAAATGCTTCAATAATGTAGCCAACCACTAGAACTATAGCTAGA |
| | | GTCCTGGGCGAACAAACGATGCTCGCCTTCCAGAAAACCGAGGAT |
| | | GCGAACCACTTCATCCGGGGTCAGCACCACCGGCAAGCGCCGCGA |
| | | CGGCCGAGGTCTTCCGATCTCCTGAAGCCAGGGCAGATCCGTGCA |
| | | CAGCACCTTGCCGTAGAAGAACAGCAAGGCCGCCAATGCCTGACG |
| | | ATGCGTGGAGACCGAAACCTTGCGCTCGTTCGCCAGCCAGGACAG |
| | | AAATGCCTCGACTTCGCTGCTGCCCAAGGTTGCCGGGTGACGCACA |
| | | CCGTGGAAACGGATGAAGGCACGAACCCAGTTGACATAAGCCTGT |
| | | TCGGTTCGTAAACTGTAATGCAAGTAGCGTATGCGCTCACGCAACT |
| | | GGTCCAGAACCTTGACCGAACGCAGCGGTGGTAACGGCGCAGTGG |
| | | CGGTTTTCATGGCTTGTTATGACTGTTTTTTTGTACAGTCTATGCCT |
| | | CGGGCATCCAAGCAGCAAGCGCGTTACGCCGTGGGTCGATGTTTG |
| | | ATGTTATGGAGCAGCAACGATGTTACGCAGCAGCAACGATGTTAC |
| | | GCAGCAGGGCAGTCGCCCTAAAACAAAGTTAGGTGGCTCAAGTAT |
| | | GGGCATCATTCGCACATGTAGGCTCGGCCCTGACCAAGTCAAATCC |
| | | ATGCGGGCTGCTCTTGATCTTTTCGGTCGTGAGTTCGGAGACGTAG |
| | | CCACCTACTCCCAACATCAGCCGGACTCCGATTACCTCGGGAACTT |
| | | GCTCCGTAGTAAGACATTCATCGCGCTTGCTGCCTTCGACCAAGAA |
| | | GCGGTTGTTGGCGCTCTCGCGGCTTACGTTCTGCCCAGGTTTGAGC |
| | | AGCCGCGTAGTGAGATCTATATCTATGATCTCGCAGTCTCCGGCGA |
| | | GCACCGGAGGCAGGGCATTGCCACCGCGCTCATCAATCTCCTCAA |
| | | GCATGAGGCCAACGCGCTTGGTGCTTATGTGATCTACGTGCAAGCA |
| | | GATTACGGTGACGATCCCGCAGTGGCTCTCTATACAAAGTTGGGCA |
| | | TACGGGAAGAAGTGATGCACTTTGATATCGACCCAAGTACCGCCA |
| | | CCTAACAATTCGTTCAAGCCGAGATCGGCTTCCCGGCCGCGGAGTT |
| | | GTTCGGTAAATTGTCACAACGCCGCGAATATAGTCTTTACCATGCC |
| | | CTTGGCCACGCCCCTCTTTAATACGACGGGCAATTTGCACTTCAGA |
| | | AAATGAAGAGTTTGCTTTAGCCATAACAAAAGTCCAGTATGCTTTT |
| | | TCACAGCATAACTGGACTGATTTCAGTTTACAACTATTCTGTCTAG |
| | | TTTAAGACTTTATTGTCATAGTTTAGATCTATTTTGTTCAGTTTAAG |
| | | ACTTTATTGTCCGCCCACACCCGCTTACGCAGGGCATCCATTTATT |
| | | ACTCAACCGTAACCGATTTTGCCAGGTTACGCGGCTGGTCTGCGGT |
| | | GTGAAATACCGCACAGATGCGTAAGGAGAAAATACCGCATCAGGC |
| | | GCTCTTCCGCTTCCTCGCTCACTGACTCGCTGCGCTCGGTCGTTCGG |
| | | CTGCGGCGAGCGGTATCAGCTCACTCAAAGGCGGTAATACGGTTA |
| | | TCCACAGAATCAGGGGATAACGCAGGAAAGAACATGTGAGCAAA |
| | | AGGCCAGCAAAAGGCCAGGAACCGTAAAAAGGCCGCGTTGCTGGC |
| | | GTTTTTCCATAGGCTCCGCCCCCCTGACGAGCATCACAAAAATCGA |
| | | CGCTCAAGTCAGAGGTGGCGAAACCCGACAGGACTATAAAGATAC |
| | | CAGGCGTTTCCCCCTGGAAGCTCCCTCGTGCGCTCTCCTGTTCCGA |
| | | CCCTGCCGCTTACCGGATACCTGTCCGCCTTTCTCCCTTCGGGAAG |
| | | CGTGGCGCTTTCTCAATGCTCACGCTGTAGGTATCTCAGTTCGGTG |
| | | TAGGTCGTTCGCTCCAAGCTGGGCTGTGTGCACGAACCCCCCGTTC |
| | | AGCCCGACCGCTGCGCCTTATCCGGTAACTATCGTCTTGAGTCCAA |
| | | CCCGGTAAGACACGACTTATCGCCACTGGCAGCAGCCACTGGTAA |
| | | CAGGATTAGCAGAGCGAGGTATGTAGGCGGTGCTACAGAGTTCTT |
| | | GAAGTGGTGGCCTAACTACGGCTACACTAGAAGGACAGTATTTGG |
| | | TATCTGCGCTCTGCTGAAGCCAGTTACCTTCGGAAAAAGAGTTGGT |
| | | AGCTCTTGATCCGGCAAACAAACCACCGCTGGTAGCGGTGGTTTTT |
| | | TTGTTTGCAAGCAGCAGATTACGCGCAGAAAAAAAGGATCTCAAG |
| | | AAGATCCTTTGATCTTTTCTACGTGGTCTGACGCTCAGTGGAACGA |
| | | AAACTCACGTTAAGGGATTTTGGTCATGAGATTATCAAAAAGGAT |
| | | CTTCACCTAGATCCTTTTAAATTAAAAATGAAGTTTTAAATCAATC |
| | | TAAAGTATATATGAGTAAACTTGGTCTGACGGTTACTCAGAAGAA |
| | | CTCGTCAAGAAGGCGATAGAAGGCGATGCGCTGCGAATCGGGAGC |
| | | GGCGATACCGTAAAGCACGAGGAAGCGGTCAGCCCATTCGCCGCC |
| | | AAGCTCTTCAGCAATATCACGGGTAGCCAACGCTATGTCCTGATAG |
| | | CGGTCCGCCACACCCAGCCGGCCACAGTCGATGAATCCAGAAAAG |
| | | CGGCCATTTTCCACCATGATATTCGGCAAGCAGGCATCGCCATGGG |
| | | TCACGACGAGATCCTCGCCGTCGGGCATGCGCGCCTTGAGCCTGGC |
| | | GAACAGTTCGGCTGGCGCGAGCCCCTGATGCTCTTCGTCCAGATCA |
| | | TCCTGATCGACAAGACCGGCTTCCATCCGAGTACGTGCTCGCTCGA |
| | | TGCGATGTTTCGCTTGGTGGTCGAATGGGCAGGTAGCCGGATCAA |
| | | GCGTATGCAGCCGCCGCATTGCATCAGCCATGATGGATACTTTCTC |
| | | GGCAGGAGCAAGGTGAGATGACAGGAGATCCTGCCCCGGCACTTC |
| | | GCCCAATAGCAGCCAGTCCCTTCCCGCTTCAGTGACAACGTCGAGC |
| | | ACAGCTGCGCAAGGAACGCCCGTCGTGGCCAGCCACGATAGCCGC |
| | | GCTGCCTCGTCCTGCAGTTCATTCAGGGCACCGGACAGGTCGGTCT |
| | | TGACAAAAAGAACCGGGCGCCCCTGCGCTGACAGCCGGAACACGG |
| | | CGGCATCAGAGCAGCCGATTGTCTGTTGTGCCCAGTCATAGCCGAA |
| | | TAGCCTCTCCACCCAAGCGGCCGGAGAACCTGCGTGCAATCCATCT |
| | | TGTTCAATCATACTCTTCCTTTTTCAATATTATTGAAGCATTTATCA |
| | | GGGTTATTGTCTCATGAGCGGATACATATTTGAATGTATTTAGAAA |
| | | AATAAACAAATAGGGGTTCCGCGCACATTTCCCCGAAAAGTGCCA |
| | | CCTGACGTCTAAGAAACCATTATTATCATGACATTAACCTATAAAA |
| | | ATAGGCGTATCACGAGGCCCTTTCGTCTCGCGCGTTTCGGTGATGA |
| | | CGGTGAAAACCTCTGACACATGCAGCTCCCGGAGACGGTCACAGC |
| | | TTGTCTGTAAGCGGATGCCGGGAGCAGACAAGCCCGTCAGGGCGC |
| | | GTCAGCGGGTGTTGGCGGGTGTCGGGGCTGGCTTAACTATGCGGC |
| | | ATCAGAGCAGATTGTACTGAGAGTGCACCATATGCGGTGTGAAAT |
| | | ACCGCACAGATGCGTAAGGAGAAAATACCGCATCAGGAAATTGTA |
| | | AACGTTAATATTTTGTTAAAATTCGCGTTAAATTTTTGTTAAATCAG |
| | | CTCATTTTTTAACCAATAGGCCGAAATCGGCAAAATCCCTTATAAA |
| | | TCAAAAGAATAGACCGAGATAGGGTTGAGTGTTGTTCCAGTTTGG |
| | | AACAAGAGTCCACTATTAAAGAACGTGGACTCCAACGTCAAAGGG |
| | | CGAAAAACCGTCTATCAGGGCGATGGCCCACTACGTGAACCATCA |
| | | CCCTAATCAAGTTTTTTGGGGTCGAGGTGCCGTAAAGCACTAAATC |
| | | GGAACCCTAAAGGGAGCCCCCGATTTAGAGCTTGACGGGGAAAGC |
| | | CGGCGAACGTGGCGAGAAAGGAAGGGAAGAAAGCGAAAGGAGCG |
| | | GGCGCTAGGGCGCTGGCAAGTGTAGCGGTCACGCTGCGCGTAACC |
| | | ACCACACCCGCCGCGCTTAATGCGCCGCTACAGGGCGCGTCC |
| SEQ ID NO: 128 | Vector | CATTCGCCATTCAGGCTGCAAATAAGCGTTGATATTCAGTCAATTA |
| | GEM | CAAACATTAATAACGAAGAGATGACAGAAAAATTTTCATTCTGTG |
| | Plasmid | ACAGAGAAAAAGTAGCCGAAGATGACGGTTTGTCACATGGAGTTG |
| | | GCAGGATGTTTGATTAAAAACATAACAGGAAGAAAAATGCCCCGC |
| | | TGTGGGCGGACAAAATAGTTGGGAACTGGGAGGGGTGGAAATGG |
| | | AGTTTTTAAGGATTATTTAGGGAAGAGTGACAAAATAGATGGGAA |
| | | CTGGGTGTAGCGTCGTAAGCTAATACGAAAATTAAAAATGACAAA |
| | | ATAGTTTGGAACTAGATTTCACTTATCTGGTTCGGATCTCCTAGGG |
| | | GTACCGGGCCCCCCCTCGAGGTCGACGGTATCGATGGATCCGATAT |
| | | CGAATTCAAGCGCGCTCGCTCGCTCACTGAGGCCGCCCGGGCAAA |
| | | GCCCGGGCGTCGGGCGACCTTTGGTCGCCCGGCCTCAGTGAGCGA |
| | | GCGAGCGCGCAGAGAGGGAGTGGCCAACTCCATCACTAGGGGTTC |
| | | CTTGTAGTTAATGATTAACCCGCCATGCTACTTATCTACGTACTCTG |
| | | GAGACGACTTACGGTAAATGGCCCGCCTGGCTGACCGCCCAACGA |
| | | CCCCCGCCCATTGACGTCAATAATGACGTATGTTCCCATAGTAACG |
| | | CCAATAGGGACTTTCCATTGACGTCAATGGGTGGAGTATTTACGGT |
| | | AAACTGCCCACTTGGCAGTACATCAAGTGTATCATATGCCAAGTCC |
| | | GCCCCCTATTGACGTCAATGACGGTAAATGGCCCGCCTGGCATTAT |
| | | GCCCAGTACATGACCTTACGGGACTTTCCTACTTGGCAGTACATCT |
| | | ACGTATTAGTCATCGCTATTACCATGGTCGAGGTGAGCCCCACGTT |
| | | CTGCTTCACTCTCCCCATCTCCCCCCCCTCCCCACCCCCAATTTTGT |
| | | ATTTATTTATTTTTTAATTATTTTGTGCAGCGATGGGGGGGGGGGG |
| | | GGGGGGGGGGGGCGCGCCAGGCGGGGCGGGGCGGGGCGAGGGGC |
| | | GGGGCGGGGCGAGGCGGAGAGGTGCGGCGGCAGCCAATCAGAGC |
| | | GGCGCGCTCCGAAAGTTTCCTTTTATGGCGAGGCGGCGGCGGCGG |
| | | CGGCCCTATAAAAAGCGAAGCGCGCGGCGGGCGGGAGTCGCTGCG |
| | | ACGCTGCCTTCGCCCCGTGCCCCGCTCCGCCGCCGCCTCGCGCCGC |
| | | CCGCCCCGGCTCTGACTGACCGCGTTACTCCCACAGAGATCTCGTT |
| | | TAGTGAACCGTCAGATCCTCACTCTCTTCCGCATCGCTGTCTGCGA |
| | | GGGCCAGCTGTTGGGCTCGCGGTTGAGGACAAACTCTTCGCGGTCT |
| | | TTCCAGTACTCTTGGATCGGAAACCCGTCGGCCTCCGAACGGTACT |
| | | CCGCCACCGAGGGACCTGAGCGAGTCCGCATCGACCGGATCGGAA |
| | | AACCTCTCGAGAAAGGCGTCTAACCAGTCACAGTCGCAAGGTAGG |
| | | CTGAGCACCGTGGCGGGCGGCAGCGGGTGGCGGTCGGGGTTGTTT |
| | | CTGGCGGAGGTGCTGCTGATGATGTAATTAAAGTAGGCGGTCTTG |
| | | AGACGGCGGATGGTCGAGGTGAGGTGTGGCAGGCTTGAGATCCAG |
| | | CTGTTGGGGTGAGTACTCCCTCTCAAAAGCGGGCATTACTTCTGCG |
| | | CTAAGATTGTCAGTTTCCAAAAACGAGGAGGATTTGATATTCACCT |
| | | GGCCCGATCTGGCCATACACTTGAGTGACAATGACATCCACTTTGC |
| | | CTTTCTCTCCACAGGTGTCCACTCCCAGGTCCAAGTTTAAACGCCG |
| | | CCACCATGGGCATCCAGGGCGGCAGCGTGCTGTTCGGCCTGCTGCT |
| | | GGTGCTGGCCGTGTTCTGCCACAGCGGCCACAGCCTGCAGTGCTAC |
| | | AACTGCCCCAACCCCACCGCCGACTGCAAGACCGCCGTGAACTGC |
| | | AGCAGCGACTTCGACGCCTGCCTGATCACCAAGGCCGGCCTGCAG |
| | | GTGTACAACAAGTGCTGGAAGTTCGAGCACTGCAACTTCAACGAC |
| | | GTGACCACCAGGCTGAGGGAGAACGAGCTGACCTACTACTGCTGC |
| | | AAGAAGGACCTGTGCAACTTCAACGAGCAGCTGGAGAACTAAGTT |
| | | AACTCGAGAATCAACCTCTGGATTACAAAATTTGTGAAAGATTGA |
| | | CTGGTATTCTTAACTATGTTGCTCCTTTTACGCTATGTGGATACGCT |
| | | GCTTTAATGCCTTTGTATCATGCTATTGCTTCCCGTATGGCTTTCAT |
| | | TTTCTCCTCCTTGTATAAATCCTGGTTGCTGTCTCTTTATGAGGAGT |
| | | TGTGGCCCGTTGTCAGGCAACGTGGCGTGGTGTGCACTGTGTTTGC |
| | | TGACGCAACCCCCACTGGTTGGGGCATTGCCACCACCTGTCAGCTC |
| | | CTTTCCGGGACTTTCGCTTTCCCCCTCCCTATTGCCACGGCGGAACT |
| | | CATCGCCGCCTGCCTTGCCCGCTGCTGGACAGGGGCTCGGCTGTTG |
| | | GGCACTGACAATTCCGTGGTCTGTTCTCATCACATCATATCAAGGT |
| | | TATATACCATCAATATTGCCACAGATGTTACTTAGCCTTTTAATATT |
| | | TCTCTAATTTAGTGTATATGCAATGATAGTTCTCTGATTTCTGAGAT |
| | | TGAGTTTCTCATGTGTAATGATTATTTAGAGTTTCTCTTTCATCTGT |
| | | TCAAATTTTTGTCTAGTTTTATTTTTTACTGATTTGTAAGACTTCTTT |
| | | TTATAATCTGCATATTACAATTCTCTTTACTGGGGTGTTGCAAATAT |
| | | TTTCTGTCATTCTATGGCCTGACTTTTCTTAATGGTTTTTTAATTTTA |
| | | AAAATAAGTCTTAATATTCATGCAATCTAATTAACAATCTTTTCTTT |
| | | GTGGTTAGGACTTTGAGTCATAAGAAATTTTTCTCTACACTGAAGT |
| | | CATGATGGCATGCTTCTATATTATTTTCTAAAAGATTTAAAGTTTTG |
| | | CCTTCTCCATTTAGACTTATAATTCACTGGAATTTTTTTGTGTGTAT |
| | | GGTATGACATATGGGTTCCCTTTTATTTTTTACATATAAATATATTT |
| | | CCCTGTTTTTCTAAAAAAGAAAAAGATCATCATTTTCCCATTGTAA |
| | | AATGCCATATTTTTTTCATAGGTCACTTACATATATCAATGGGTCTG |
| | | TTTCTGAGCTCTACTCTATTTTATCAGCCTCACTGTCTATCCCCACA |
| | | CATCTCATGCTTTGCTCTAAATCTTGATATTTAGTGGAACATTCTTT |
| | | CCCATTTTGTTCTACAAGAATATTTTTGTTATTGTCTTTGGGCTTTC |
| | | TATATACATTTTGAAATGAGGTTGACAAGTTACCTAGGAAAACTGT |
| | | CTTCCTGCCCGGGTGGCATCCCTGTGACCCCTCCCCAGTGCCTCTC |
| | | CTGGCCCTGGAAGTTGCCACTCCAGTGCCCACCAGCCTTGTCCTAA |
| | | TAAAATTAAGTTGCATCATTTTGTCTGACTAGGTGTCCTTCTATAAT |
| | | ATTATGGGGTGGAGGGGGGTGGTATGGAGCAAGGGGCCCAAGTTG |
| | | GGAAGAAACCTGTAGGGCCTGCGTACGTAGATAAGTAGCATGGCG |
| | | GGTTAATCATTAACTACAAGGAACCCCTAGTGATGGAGTTGGCCA |
| | | CTCCCTCTCTGCGCGCTCGCTCGCTCACTGAGGCCGGGCGACCAAA |
| | | GGTCGCCCGACGCCCGGGCTTTGCCCGGGCGGCCTCAGTGAGCGA |
| | | GCGAGCGCGCAGCTGCTGGTACCGGGCCCCCCCTCGAGGTCGACG |
| | | GTATCGATGGATCCGATATCGAATTCAACCTGCAGGCATGCTCTGA |
| | | GACAATAACCCTGATAAATGCTTCAATAATGTAGCCAACCACTAG |
| | | AACTATAGCTAGAGTCCTGGGCGAACAAACGATGCTCGCCTTCCA |
| | | GAAAACCGAGGATGCGAACCACTTCATCCGGGGTCAGCACCACCG |
| | | GCAAGCGCCGCGACGGCCGAGGTCTTCCGATCTCCTGAAGCCAGG |
| | | GCAGATCCGTGCACAGCACCTTGCCGTAGAAGAACAGCAAGGCCG |
| | | CCAATGCCTGACGATGCGTGGAGACCGAAACCTTGCGCTCGTTCGC |
| | | CAGCCAGGACAGAAATGCCTCGACTTCGCTGCTGCCCAAGGTTGC |
| | | CGGGTGACGCACACCGTGGAAACGGATGAAGGCACGAACCCAGTT |
| | | GACATAAGCCTGTTCGGTTCGTAAACTGTAATGCAAGTAGCGTATG |
| | | CGCTCACGCAACTGGTCCAGAACCTTGACCGAACGCAGCGGTGGT |
| | | AACGGCGCAGTGGCGGTTTTCATGGCTTGTTATGACTGTTTTTTTGT |
| | | ACAGTCTATGCCTCGGGCATCCAAGCAGCAAGCGCGTTACGCCGT |
| | | GGGTCGATGTTTGATGTTATGGAGCAGCAACGATGTTACGCAGCA |
| | | GCAACGATGTTACGCAGCAGGGCAGTCGCCCTAAAACAAAGTTAG |
| | | GTGGCTCAAGTATGGGCATCATTCGCACATGTAGGCTCGGCCCTGA |
| | | CCAAGTCAAATCCATGCGGGCTGCTCTTGATCTTTTCGGTCGTGAG |
| | | TTCGGAGACGTAGCCACCTACTCCCAACATCAGCCGGACTCCGATT |
| | | ACCTCGGGAACTTGCTCCGTAGTAAGACATTCATCGCGCTTGCTGC |
| | | CTTCGACCAAGAAGCGGTTGTTGGCGCTCTCGCGGCTTACGTTCTG |
| | | CCCAGGTTTGAGCAGCCGCGTAGTGAGATCTATATCTATGATCTCG |
| | | CAGTCTCCGGCGAGCACCGGAGGCAGGGCATTGCCACCGCGCTCA |
| | | TCAATCTCCTCAAGCATGAGGCCAACGCGCTTGGTGCTTATGTGAT |
| | | CTACGTGCAAGCAGATTACGGTGACGATCCCGCAGTGGCTCTCTAT |
| | | ACAAAGTTGGGCATACGGGAAGAAGTGATGCACTTTGATATCGAC |
| | | CCAAGTACCGCCACCTAACAATTCGTTCAAGCCGAGATCGGCTTCC |
| | | CGGCCGCGGAGTTGTTCGGTAAATTGTCACAACGCCGCGAATATA |
| | | GTCTTTACCATGCCCTTGGCCACGCCCCTCTTTAATACGACGGGCA |
| | | ATTTGCACTTCAGAAAATGAAGAGTTTGCTTTAGCCATAACAAAAG |
| | | TCCAGTATGCTTTTTCACAGCATAACTGGACTGATTTCAGTTTACA |
| | | ACTATTCTGTCTAGTTTAAGACTTTATTGTCATAGTTTAGATCTATT |
| | | TTGTTCAGTTTAAGACTTTATTGTCCGCCCACACCCGCTTACGCAG |
| | | GGCATCCATTTATTACTCAACCGTAACCGATTTTGCCAGGTTACGC |
| | | GGCTGGTCTGCGGTGTGAAATACCGCACAGATGCGTAAGGAGAAA |
| | | ATACCGCATCAGGCGCTCTTCCGCTTCCTCGCTCACTGACTCGCTG |
| | | CGCTCGGTCGTTCGGCTGCGGCGAGCGGTATCAGCTCACTCAAAG |
| | | GCGGTAATACGGTTATCCACAGAATCAGGGGATAACGCAGGAAAG |
| | | AACATGTGAGCAAAAGGCCAGCAAAAGGCCAGGAACCGTAAAAA |
| | | GGCCGCGTTGCTGGCGTTTTTCCATAGGCTCCGCCCCCCTGACGAG |
| | | CATCACAAAAATCGACGCTCAAGTCAGAGGTGGCGAAACCCGACA |
| | | GGACTATAAAGATACCAGGCGTTTCCCCCTGGAAGCTCCCTCGTGC |
| | | GCTCTCCTGTTCCGACCCTGCCGCTTACCGGATACCTGTCCGCCTTT |
| | | CTCCCTTCGGGAAGCGTGGCGCTTTCTCAATGCTCACGCTGTAGGT |
| | | ATCTCAGTTCGGTGTAGGTCGTTCGCTCCAAGCTGGGCTGTGTGCA |
| | | CGAACCCCCCGTTCAGCCCGACCGCTGCGCCTTATCCGGTAACTAT |
| | | CGTCTTGAGTCCAACCCGGTAAGACACGACTTATCGCCACTGGCAG |
| | | CAGCCACTGGTAACAGGATTAGCAGAGCGAGGTATGTAGGCGGTG |
| | | CTACAGAGTTCTTGAAGTGGTGGCCTAACTACGGCTACACTAGAA |
| | | GGACAGTATTTGGTATCTGCGCTCTGCTGAAGCCAGTTACCTTCGG |
| | | AAAAAGAGTTGGTAGCTCTTGATCCGGCAAACAAACCACCGCTGG |
| | | TAGCGGTGGTTTTTTTGTTTGCAAGCAGCAGATTACGCGCAGAAAA |
| | | AAAGGATCTCAAGAAGATCCTTTGATCTTTTCTACGTGGTCTGACG |
| | | CTCAGTGGAACGAAAACTCACGTTAAGGGATTTTGGTCATGAGATT |
| | | ATCAAAAAGGATCTTCACCTAGATCCTTTTAAATTAAAAATGAAGT |
| | | TTTAAATCAATCTAAAGTATATATGAGTAAACTTGGTCTGACGGTT |
| | | ACTCAGAAGAACTCGTCAAGAAGGCGATAGAAGGCGATGCGCTGC |
| | | GAATCGGGAGCGGCGATACCGTAAAGCACGAGGAAGCGGTCAGC |
| | | CCATTCGCCGCCAAGCTCTTCAGCAATATCACGGGTAGCCAACGCT |
| | | ATGTCCTGATAGCGGTCCGCCACACCCAGCCGGCCACAGTCGATG |
| | | AATCCAGAAAAGCGGCCATTTTCCACCATGATATTCGGCAAGCAG |
| | | GCATCGCCATGGGTCACGACGAGATCCTCGCCGTCGGGCATGCGC |
| | | GCCTTGAGCCTGGCGAACAGTTCGGCTGGCGCGAGCCCCTGATGCT |
| | | CTTCGTCCAGATCATCCTGATCGACAAGACCGGCTTCCATCCGAGT |
| | | ACGTGCTCGCTCGATGCGATGTTTCGCTTGGTGGTCGAATGGGCAG |
| | | GTAGCCGGATCAAGCGTATGCAGCCGCCGCATTGCATCAGCCATG |
| | | ATGGATACTTTCTCGGCAGGAGCAAGGTGAGATGACAGGAGATCC |
| | | TGCCCCGGCACTTCGCCCAATAGCAGCCAGTCCCTTCCCGCTTCAG |
| | | TGACAACGTCGAGCACAGCTGCGCAAGGAACGCCCGTCGTGGCCA |
| | | GCCACGATAGCCGCGCTGCCTCGTCCTGCAGTTCATTCAGGGCACC |
| | | GGACAGGTCGGTCTTGACAAAAAGAACCGGGCGCCCCTGCGCTGA |
| | | CAGCCGGAACACGGCGGCATCAGAGCAGCCGATTGTCTGTTGTGC |
| | | CCAGTCATAGCCGAATAGCCTCTCCACCCAAGCGGCCGGAGAACC |
| | | TGCGTGCAATCCATCTTGTTCAATCATACTCTTCCTTTTTCAATATT |
| | | ATTGAAGCATTTATCAGGGTTATTGTCTCATGAGCGGATACATATT |
| | | TGAATGTATTTAGAAAAATAAACAAATAGGGGTTCCGCGCACATT |
| | | TCCCCGAAAAGTGCCACCTGACGTCTAAGAAACCATTATTATCATG |
| | | ACATTAACCTATAAAAATAGGCGTATCACGAGGCCCTTTCGTCTCG |
| | | CGCGTTTCGGTGATGACGGTGAAAACCTCTGACACATGCAGCTCCC |
| | | GGAGACGGTCACAGCTTGTCTGTAAGCGGATGCCGGGAGCAGACA |
| | | AGCCCGTCAGGGCGCGTCAGCGGGTGTTGGCGGGTGTCGGGGCTG |
| | | GCTTAACTATGCGGCATCAGAGCAGATTGTACTGAGAGTGCACCA |
| | | TATGCGGTGTGAAATACCGCACAGATGCGTAAGGAGAAAATACCG |
| | | CATCAGGAAATTGTAAACGTTAATATTTTGTTAAAATTCGCGTTAA |
| | | ATTTTTGTTAAATCAGCTCATTTTTTAACCAATAGGCCGAAATCGG |
| | | CAAAATCCCTTATAAATCAAAAGAATAGACCGAGATAGGGTTGAG |
| | | TGTTGTTCCAGTTTGGAACAAGAGTCCACTATTAAAGAACGTGGAC |
| | | TCCAACGTCAAAGGGCGAAAAACCGTCTATCAGGGCGATGGCCCA |
| | | CTACGTGAACCATCACCCTAATCAAGTTTTTTGGGGTCGAGGTGCC |
| | | GTAAAGCACTAAATCGGAACCCTAAAGGGAGCCCCCGATTTAGAG |
| | | CTTGACGGGGAAAGCCGGCGAACGTGGCGAGAAAGGAAGGGAAG |
| | | AAAGCGAAAGGAGCGGGCGCTAGGGCGCTGGCAAGTGTAGCGGT |
| | | CACGCTGCGCGTAACCACCACACCCGCCGCGCTTAATGCGCCGCTA |
| | | CAGGGCGCGTCC |
| SEQ ID NO: 129 | Vector | CATTCGCCATTCAGGCTGCAAATAAGCGTTGATATTCAGTCAATTA |
| | GEP | CAAACATTAATAACGAAGAGATGACAGAAAAATTTTCATTCTGTG |
| | Plasmid | ACAGAGAAAAAGTAGCCGAAGATGACGGTTTGTCACATGGAGTTG |
| | | GCAGGATGTTTGATTAAAAACATAACAGGAAGAAAAATGCCCCGC |
| | | TGTGGGCGGACAAAATAGTTGGGAACTGGGAGGGGTGGAAATGG |
| | | AGTTTTTAAGGATTATTTAGGGAAGAGTGACAAAATAGATGGGAA |
| | | CTGGGTGTAGCGTCGTAAGCTAATACGAAAATTAAAAATGACAAA |
| | | ATAGTTTGGAACTAGATTTCACTTATCTGGTTCGGATCTCCTAGGG |
| | | GTACCGGGCCCCCCCTCGAGGTCGACGGTATCGATGGATCCGATAT |
| | | CGAATTCAAGCGCGCTCGCTCGCTCACTGAGGCCGCCCGGGCAAA |
| | | GCCCGGGCGTCGGGCGACCTTTGGTCGCCCGGCCTCAGTGAGCGA |
| | | GCGAGCGCGCAGAGAGGGAGTGGCCAACTCCATCACTAGGGGTTC |
| | | CTTGTAGTTAATGATTAACCCGCCATGCTACTTATCTACGTACTCTG |
| | | GAGACGACTTACGGTAAATGGCCCGCCTGGCTGACCGCCCAACGA |
| | | CCCCCGCCCATTGACGTCAATAATGACGTATGTTCCCATAGTAACG |
| | | CCAATAGGGACTTTCCATTGACGTCAATGGGTGGAGTATTTACGGT |
| | | AAACTGCCCACTTGGCAGTACATCAAGTGTATCATATGCCAAGTCC |
| | | GCCCCCTATTGACGTCAATGACGGTAAATGGCCCGCCTGGCATTAT |
| | | GCCCAGTACATGACCTTACGGGACTTTCCTACTTGGCAGTACATCT |
| | | ACGTATTAGTCATCGCTATTACCATGGTCGAGGTGAGCCCCACGTT |
| | | CTGCTTCACTCTCCCCATCTCCCCCCCCTCCCCACCCCCAATTTTGT |
| | | ATTTATTTATTTTTTAATTATTTTGTGCAGCGATGGGGGCGGGGGG |
| | | GGGGGGGGGGGGCGCGCCAGGCGGGGCGGGGCGGGGCGAGGGGC |
| | | GGGGCGGGGCGAGGCGGAGAGGTGCGGCGGCAGCCAATCAGAGC |
| | | GGCGCGCTCCGAAAGTTTCCTTTTATGGCGAGGCGGCGGCGGCGG |
| | | CGGCCCTATAAAAAGCGAAGCGCGCGGCGGGCGGGAGTCGCTGCG |
| | | ACGCTGCCTTCGCCCCGTGCCCCGCTCCGCCGCCGCCTCGCGCCGC |
| | | CCGCCCCGGCTCTGACTGACCGCGTTACTCCCACAGAGATCTCGTT |
| | | TAGTGAACCGTCAGATCCTCACTCTCTTCCGCATCGCTGTCTGCGA |
| | | GGGCCAGCTGTTGGGCTCGCGGTTGAGGACAAACTCTTCGCGGTCT |
| | | TTCCAGTACTCTTGGATCGGAAACCCGTCGGCCTCCGAACGGTACT |
| | | CCGCCACCGAGGGACCTGAGCGAGTCCGCATCGACCGGATCGGAA |
| | | AACCTCTCGAGAAAGGCGTCTAACCAGTCACAGTCGCAAGGTAGG |
| | | CTGAGCACCGTGGCGGGCGGCAGCGGGTGGCGGTCGGGGTTGTTT |
| | | CTGGCGGAGGTGCTGCTGATGATGTAATTAAAGTAGGCGGTCTTG |
| | | AGACGGCGGATGGTCGAGGTGAGGTGTGGCAGGCTTGAGATCCAG |
| | | CTGTTGGGGTGAGTACTCCCTCTCAAAAGCGGGCATTACTTCTGCG |
| | | CTAAGATTGTCAGTTTCCAAAAACGAGGAGGATTTGATATTCACCT |
| | | GGCCCGATCTGGCCATACACTTGAGTGACAATGACATCCACTTTGC |
| | | CTTTCTCTCCACAGGTGTCCACTCCCAGGTCCAAGTTTAAACGCCG |
| | | CCACCATGGAGTTCGGCCTGAGCTGGCTGTTCCTGGTGGCCATCCT |
| | | TAAGGGCGTGCAGTGCGAGGTGCAGCTGCAGTGCTACAACTGCCC |
| | | CAACCCCACCGCCGACTGCAAGACCGCCGTGAACTGCAGCAGCGA |
| | | CTTCGACGCCTGCCTGATCACCAAGGCCGGCCTGCAGGTGTACAAC |
| | | AAGTGCTGGAAGTTCGAGCACTGCAACTTCAACGACGTGACCACC |
| | | AGGCTGAGGGAGAACGAGCTGACCTACTACTGCTGCAAGAAGGAC |
| | | CTGTGCAACTTCAACGAGCAGCTGGAGAACTAAGTTAACTCGAGA |
| | | ATCAACCTCTGGATTACAAAATTTGTGAAAGATTGACTGGTATTCT |
| | | TAACTATGTTGCTCCTTTTACGCTATGTGGATACGCTGCTTTAATGC |
| | | CTTTGTATCATGCTATTGCTTCCCGTATGGCTTTCATTTTCTCCTCCT |
| | | TGTATAAATCCTGGTTGCTGTCTCTTTATGAGGAGTTGTGGCCCGTT |
| | | GTCAGGCAACGTGGCGTGGTGTGCACTGTGTTTGCTGACGCAACCC |
| | | CCACTGGTTGGGGCATTGCCACCACCTGTCAGCTCCTTTCCGGGAC |
| | | TTTCGCTTTCCCCCTCCCTATTGCCACGGCGGAACTCATCGCCGCCT |
| | | GCCTTGCCCGCTGCTGGACAGGGGCTCGGCTGTTGGGCACTGACA |
| | | ATTCCGTGGTCTGTTCTCATCACATCATATCAAGGTTATATACCATC |
| | | AATATTGCCACAGATGTTACTTAGCCTTTTAATATTTCTCTAATTTA |
| | | GTGTATATGCAATGATAGTTCTCTGATTTCTGAGATTGAGTTTCTCA |
| | | TGTGTAATGATTATTTAGAGTTTCTCTTTCATCTGTTCAAATTTTTG |
| | | TCTAGTTTTATTTTTTACTGATTTGTAAGACTTCTTTTTATAATCTGC |
| | | ATATTACAATTCTCTTTACTGGGGTGTTGCAAATATTTTCTGTCATT |
| | | CTATGGCCTGACTTTTCTTAATGGTTTTTTAATTTTAAAAATAAGTC |
| | | TTAATATTCATGCAATCTAATTAACAATCTTTTCTTTGTGGTTAGGA |
| | | CTTTGAGTCATAAGAAATTTTTCTCTACACTGAAGTCATGATGGCA |
| | | TGCTTCTATATTATTTTCTAAAAGATTTAAAGTTTTGCCTTCTCCAT |
| | | TTAGACTTATAATTCACTGGAATTTTTTTGTGTGTATGGTATGACAT |
| | | ATGGGTTCCCTTTTATTTTTTACATATAAATATATTTCCCTGTTTTTC |
| | | TAAAAAAGAAAAAGATCATCATTTTCCCATTGTAAAATGCCATATT |
| | | TTTTTCATAGGTCACTTACATATATCAATGGGTCTGTTTCTGAGCTC |
| | | TACTCTATTTTATCAGCCTCACTGTCTATCCCCACACATCTCATGCT |
| | | TTGCTCTAAATCTTGATATTTAGTGGAACATTCTTTCCCATTTTGTT |
| | | CTACAAGAATATTTTTGTTATTGTCTTTGGGCTTTCTATATACATTT |
| | | TGAAATGAGGTTGACAAGTTACCTAGGAAAACTGTCTTCCTGCCCG |
| | | GGTGGCATCCCTGTGACCCCTCCCCAGTGCCTCTCCTGGCCCTGGA |
| | | AGTTGCCACTCCAGTGCCCACCAGCCTTGTCCTAATAAAATTAAGT |
| | | TGCATCATTTTGTCTGACTAGGTGTCCTTCTATAATATTATGGGGTG |
| | | GAGGGGGGTGGTATGGAGCAAGGGGCCCAAGTTGGGAAGAAACC |
| | | TGTAGGGCCTGCGTACGTAGATAAGTAGCATGGCGGGTTAATCATT |
| | | AACTACAAGGAACCCCTAGTGATGGAGTTGGCCACTCCCTCTCTGC |
| | | GCGCTCGCTCGCTCACTGAGGCCGGGCGACCAAAGGTCGCCCGAC |
| | | GCCCGGGCTTTGCCCGGGCGGCCTCAGTGAGCGAGCGAGCGCGCA |
| | | GCTGCTGGTACCGGGCCCCCCCTCGAGGTCGACGGTATCGATGGAT |
| | | CCGATATCGAATTCAACCTGCAGGCATGCTCTGAGACAATAACCCT |
| | | GATAAATGCTTCAATAATGTAGCCAACCACTAGAACTATAGCTAG |
| | | AGTCCTGGGCGAACAAACGATGCTCGCCTTCCAGAAAACCGAGGA |
| | | TGCGAACCACTTCATCCGGGGTCAGCACCACCGGCAAGCGCCGCG |
| | | ACGGCCGAGGTCTTCCGATCTCCTGAAGCCAGGGCAGATCCGTGC |
| | | ACAGCACCTTGCCGTAGAAGAACAGCAAGGCCGCCAATGCCTGAC |
| | | GATGCGTGGAGACCGAAACCTTGCGCTCGTTCGCCAGCCAGGACA |
| | | GAAATGCCTCGACTTCGCTGCTGCCCAAGGTTGCCGGGTGACGCAC |
| | | ACCGTGGAAACGGATGAAGGCACGAACCCAGTTGACATAAGCCTG |
| | | TTCGGTTCGTAAACTGTAATGCAAGTAGCGTATGCGCTCACGCAAC |
| | | TGGTCCAGAACCTTGACCGAACGCAGCGGTGGTAACGGCGCAGTG |
| | | GCGGTTTTCATGGCTTGTTATGACTGTTTTTTTGTACAGTCTATGCC |
| | | TCGGGCATCCAAGCAGCAAGCGCGTTACGCCGTGGGTCGATGTTT |
| | | GATGTTATGGAGCAGCAACGATGTTACGCAGCAGCAACGATGTTA |
| | | CGCAGCAGGGCAGTCGCCCTAAAACAAAGTTAGGTGGCTCAAGTA |
| | | TGGGCATCATTCGCACATGTAGGCTCGGCCCTGACCAAGTCAAATC |
| | | CATGCGGGCTGCTCTTGATCTTTTCGGTCGTGAGTTCGGAGACGTA |
| | | GCCACCTACTCCCAACATCAGCCGGACTCCGATTACCTCGGGAACT |
| | | TGCTCCGTAGTAAGACATTCATCGCGCTTGCTGCCTTCGACCAAGA |
| | | AGCGGTTGTTGGCGCTCTCGCGGCTTACGTTCTGCCCAGGTTTGAG |
| | | CAGCCGCGTAGTGAGATCTATATCTATGATCTCGCAGTCTCCGGCG |
| | | AGCACCGGAGGCAGGGCATTGCCACCGCGCTCATCAATCTCCTCA |
| | | AGCATGAGGCCAACGCGCTTGGTGCTTATGTGATCTACGTGCAAGC |
| | | AGATTACGGTGACGATCCCGCAGTGGCTCTCTATACAAAGTTGGGC |
| | | ATACGGGAAGAAGTGATGCACTTTGATATCGACCCAAGTACCGCC |
| | | ACCTAACAATTCGTTCAAGCCGAGATCGGCTTCCCGGCCGCGGAGT |
| | | TGTTCGGTAAATTGTCACAACGCCGCGAATATAGTCTTTACCATGC |
| | | CCTTGGCCACGCCCCTCTTTAATACGACGGGCAATTTGCACTTCAG |
| | | AAAATGAAGAGTTTGCTTTAGCCATAACAAAAGTCCAGTATGCTTT |
| | | TTCACAGCATAACTGGACTGATTTCAGTTTACAACTATTCTGTCTA |
| | | GTTTAAGACTTTATTGTCATAGTTTAGATCTATTTTGTTCAGTTTAA |
| | | GACTTTATTGTCCGCCCACACCCGCTTACGCAGGGCATCCATTTAT |
| | | TACTCAACCGTAACCGATTTTGCCAGGTTACGCGGCTGGTCTGCGG |
| | | TGTGAAATACCGCACAGATGCGTAAGGAGAAAATACCGCATCAGG |
| | | CGCTCTTCCGCTTCCTCGCTCACTGACTCGCTGCGCTCGGTCGTTCG |
| | | GCTGCGGCGAGCGGTATCAGCTCACTCAAAGGCGGTAATACGGTT |
| | | ATCCACAGAATCAGGGGATAACGCAGGAAAGAACATGTGAGCAA |
| | | AAGGCCAGCAAAAGGCCAGGAACCGTAAAAAGGCCGCGTTGCTG |
| | | GCGTTTTTCCATAGGCTCCGCCCCCCTGACGAGCATCACAAAAATC |
| | | GACGCTCAAGTCAGAGGTGGCGAAACCCGACAGGACTATAAAGAT |
| | | ACCAGGCGTTTCCCCCTGGAAGCTCCCTCGTGCGCTCTCCTGTTCC |
| | | GACCCTGCCGCTTACCGGATACCTGTCCGCCTTTCTCCCTTCGGGA |
| | | AGCGTGGCGCTTTCTCAATGCTCACGCTGTAGGTATCTCAGTTCGG |
| | | TGTAGGTCGTTCGCTCCAAGCTGGGCTGTGTGCACGAACCCCCCGT |
| | | TCAGCCCGACCGCTGCGCCTTATCCGGTAACTATCGTCTTGAGTCC |
| | | AACCCGGTAAGACACGACTTATCGCCACTGGCAGCAGCCACTGGT |
| | | AACAGGATTAGCAGAGCGAGGTATGTAGGCGGTGCTACAGAGTTC |
| | | TTGAAGTGGTGGCCTAACTACGGCTACACTAGAAGGACAGTATTT |
| | | GGTATCTGCGCTCTGCTGAAGCCAGTTACCTTCGGAAAAAGAGTTG |
| | | GTAGCTCTTGATCCGGCAAACAAACCACCGCTGGTAGCGGTGGTTT |
| | | TTTTGTTTGCAAGCAGCAGATTACGCGCAGAAAAAAAGGATCTCA |
| | | AGAAGATCCTTTGATCTTTTCTACGTGGTCTGACGCTCAGTGGAAC |
| | | GAAAACTCACGTTAAGGGATTTTGGTCATGAGATTATCAAAAAGG |
| | | ATCTTCACCTAGATCCTTTTAAATTAAAAATGAAGTTTTAAATCAA |
| | | TCTAAAGTATATATGAGTAAACTTGGTCTGACGGTTACTCAGAAGA |
| | | ACTCGTCAAGAAGGCGATAGAAGGCGATGCGCTGCGAATCGGGAG |
| | | CGGCGATACCGTAAAGCACGAGGAAGCGGTCAGCCCATTCGCCGC |
| | | CAAGCTCTTCAGCAATATCACGGGTAGCCAACGCTATGTCCTGATA |
| | | GCGGTCCGCCACACCCAGCCGGCCACAGTCGATGAATCCAGAAAA |
| | | GCGGCCATTTTCCACCATGATATTCGGCAAGCAGGCATCGCCATGG |
| | | GTCACGACGAGATCCTCGCCGTCGGGCATGCGCGCCTTGAGCCTG |
| | | GCGAACAGTTCGGCTGGCGCGAGCCCCTGATGCTCTTCGTCCAGAT |
| | | CATCCTGATCGACAAGACCGGCTTCCATCCGAGTACGTGCTCGCTC |
| | | GATGCGATGTTTCGCTTGGTGGTCGAATGGGCAGGTAGCCGGATC |
| | | AAGCGTATGCAGCCGCCGCATTGCATCAGCCATGATGGATACTTTC |
| | | TCGGCAGGAGCAAGGTGAGATGACAGGAGATCCTGCCCCGGCACT |
| | | TCGCCCAATAGCAGCCAGTCCCTTCCCGCTTCAGTGACAACGTCGA |
| | | GCACAGCTGCGCAAGGAACGCCCGTCGTGGCCAGCCACGATAGCC |
| | | GCGCTGCCTCGTCCTGCAGTTCATTCAGGGCACCGGACAGGTCGGT |
| | | CTTGACAAAAAGAACCGGGCGCCCCTGCGCTGACAGCCGGAACAC |
| | | GGCGGCATCAGAGCAGCCGATTGTCTGTTGTGCCCAGTCATAGCCG |
| | | AATAGCCTCTCCACCCAAGCGGCCGGAGAACCTGCGTGCAATCCA |
| | | TCTTGTTCAATCATACTCTTCCTTTTTCAATATTATTGAAGCATTTA |
| | | TCAGGGTTATTGTCTCATGAGCGGATACATATTTGAATGTATTTAG |
| | | AAAAATAAACAAATAGGGGTTCCGCGCACATTTCCCCGAAAAGTG |
| | | CCACCTGACGTCTAAGAAACCATTATTATCATGACATTAACCTATA |
| | | AAAATAGGCGTATCACGAGGCCCTTTCGTCTCGCGCGTTTCGGTGA |
| | | TGACGGTGAAAACCTCTGACACATGCAGCTCCCGGAGACGGTCAC |
| | | AGCTTGTCTGTAAGCGGATGCCGGGAGCAGACAAGCCCGTCAGGG |
| | | CGCGTCAGCGGGTGTTGGCGGGTGTCGGGGCTGGCTTAACTATGC |
| | | GGCATCAGAGCAGATTGTACTGAGAGTGCACCATATGCGGTGTGA |
| | | AATACCGCACAGATGCGTAAGGAGAAAATACCGCATCAGGAAATT |
| | | GTAAACGTTAATATTTTGTTAAAATTCGCGTTAAATTTTTGTTAAAT |
| | | CAGCTCATTTTTTAACCAATAGGCCGAAATCGGCAAAATCCCTTAT |
| | | AAATCAAAAGAATAGACCGAGATAGGGTTGAGTGTTGTTCCAGTT |
| | | TGGAACAAGAGTCCACTATTAAAGAACGTGGACTCCAACGTCAAA |
| | | GGGCGAAAAACCGTCTATCAGGGCGATGGCCCACTACGTGAACCA |
| | | TCACCCTAATCAAGTTTTTTGGGGTCGAGGTGCCGTAAAGCACTAA |
| | | ATCGGAACCCTAAAGGGAGCCCCCGATTTAGAGCTTGACGGGGAA |
| | | AGCCGGCGAACGTGGCGAGAAAGGAAGGGAAGAAAGCGAAAGGA |
| | | GCGGGCGCTAGGGCGCTGGCAAGTGTAGCGGTCACGCTGCGCGTA |
| | | ACCACCACACCCGCCGCGCTTAATGCGCCGCTACAGGGCGCGTCC |

TABLE 9
Peptides and fusion protein sequences of Example 4

| SEQ | | | |
|---|---|---|---|
| ID | Clone | | |
| NO | ID | Protein | Protein sequences |
| 43 | Vector | Membrane | LQCYNCPNPTADCKTAVNCSSDFDACLI |
| | GTM, | anchored | TKAGLQVYNKCWKFEHCNFNDVTTRLRE |
| | Vector | CD59 | NELTYYCCKKDLCNFNEQLENGGTSLSE |
| | GTP, | (mCD59) | KTVLLLVTPFLAAAWSLHP |
| | Vector | | |
| | GTQ, | | |
| | Vector | | |
| | GTR, | | |
| | Vector | | |
| | GAT | | |
| 44 | Vector | Native | LQCYNCPNPTADCKTAVNCSSDFDACLI |
| | GCK, | soluble | TKAGLQVYNKCWKFEHCNFNDVTTRLRE |
| | Vector | CD59 | NELTYYCCKKDLCNFNEQLEN |
| | GEM | (nsCD59) | |
| 45 | Vector | Vh- | EVQLQCYNCPNPTADCKTAVNCSSDFDA |
| | GCM, | soluble | CLITKAGLQVYNKCWKFEHCNFNDVTTR |
| | Vector | CD59, | LRENELTYYCCKKDLCNFNEQLEN |
| | GEP | (vsCD59) | |

### Example 5. Development of Macular Selective Adeno-Associated Vectors (AAV) Carrying Multiple Genes of Interests to Target Dry Age-Related Macular Degeneration

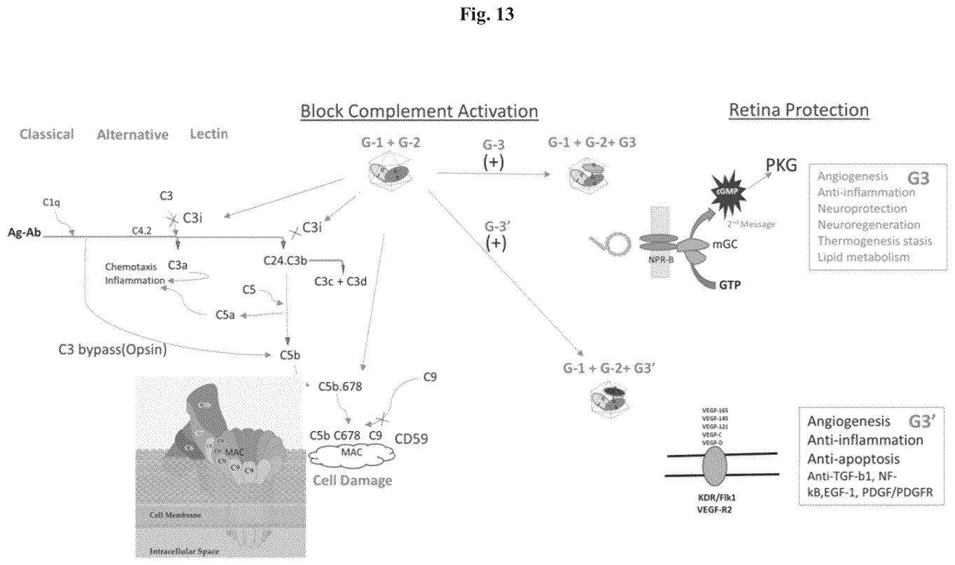

Age-related macular degeneration (AMD) is the leading cause of blindness in individuals over 60 years old, affecting 19.8 million people in the U.S. and 198 million worldwide. Currently, there is no effective treatment to halt geographic atrophy (GA) and improve visual acuity. Adeno-associated viral (AAV) vector can be a gene delivery method. AAV capsid can be engineered to improve tissue tropism. In this example, a capsid (AAV2.N54) with improved tropism to the macular retina was identified through multi-species screening in mice, pigs, rabbits, and monkeys. Across four species, AAV2.N54 showed improved tropism over wild-type AAV2 and AAV2.7m8, effectively delivering to the macular retina while detargeting from retinal ganglion cells (RGCs). The AAV2.N54 carrying multiple genes of interest (GOIs) encoding: a complement 3 inhibiting protein (C3IP), an engineered C3/C3b inhibitory peptide (C3ip) fused to human IgG Fc fragment, C3IP fused at its terminus to C3ip (C3IP-C3ip), C3IP fused at its C-terminus either with a 36 amino acid peptide containing C-type natriuretic peptide (CNP36) (C3IP-CNP36) or endostatin (C3IP-Endo), and soluble CD59 (sCD59). The resulting AAV vectors selected for systemic analysis were shown in FIG. 13: AAV2.N54-C3IP-C3ip, sCD59, AAV2.N54-C3IP-CNP36, sCD59, and AAV2.N54-C3IP-endo, sCD59. C3IP demonstrated similar affinity (KD) to C3b determined by Biacore and ELISA, comparable to the positive control peptide CP40. C3IP-C3ip showed a 2× lower IC50 than CP40 in hemolysis assays using human red blood cells (RBCs). Both C3IP-CNP36 and C3IP-Endo demonstrated hemolysis inhibition and proliferation inhibition of HUVEC cells stimulated with VEGF-A165. C3IP-CNP36 also induced cyclic guanidine monophosphate (cGMP) formation, indicating binding to natriuretic peptide receptor B (NPR-B). Fc-CNP induced a dose-dependent RGC protection in a rat model of partial optical nerve transection (pONT) and a mouse N-methyl-D-aspartate (NMDA) excitatory model following a single dose of AAV intravitreal (IVT) injection. AAV2.N54-C3IP, sCD59, AAV2.N54-C3IP-CNP36, sCD59, and AAV2.N54-C3IP-Endo, sCD59 demonstrated good expression in vitro in aRPE-19 and HEK293 cells and in mice post IVT injection. These AAV vectors are under evaluation in a sodium iodate-impaired retina model using cynomolgus monkeys.

### Example 6. Optimization of Fusion Protein Expression

Five different strategies shown in the Table 5 were applied to further optimize the C3i-Fc4-CNP36 fusion gene in the Vector GAM expression, such as, 1) The promoter was changed from CBA to CAG (Vector GGQ); 2) The recognition site of leader sequences of EVQL was replaced by DK (Vector GGE); 3) C3i location was switched from C-terminal to N-terminal (Vector GGG); 4) Fc fragment of IgG4 was replaced with Fc fragment of IgG1 (Vector GKK); 5) In the 3′ end, GOI was changed from CNP36 to endostatin (Vector GKA). The ORF sequences of GOI and their encoded protein sequences are listed in Table 19 and Table 20, respectively. Additionally, the structure of 1. Vector GGE, 2. Vector GGG, 3. Vector GGQ, 4. Vector GKA, and 5. Vector GKK are shown the FIG. 16.

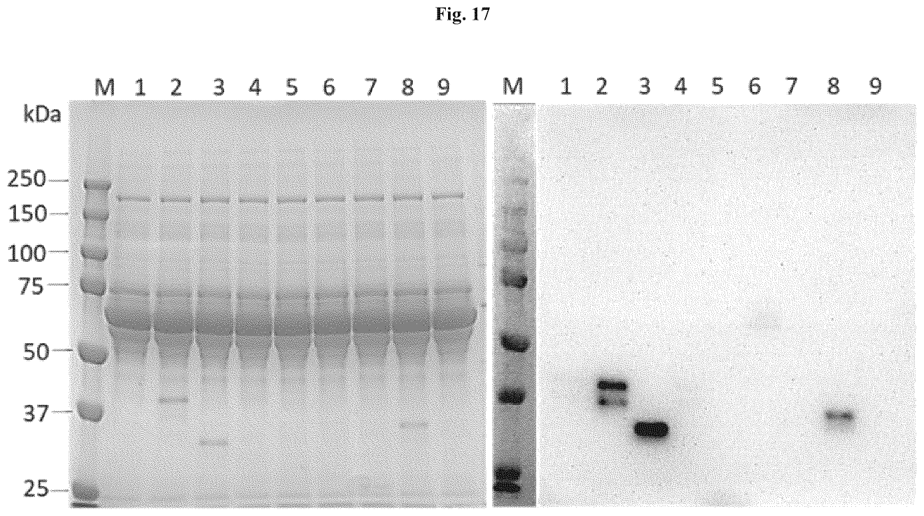

While the plasmids were prepared by Xtra Maxi Plus EF (Macherey-Nagel SAS, France), they were confirmed by restriction digestion and partial plasmid sequencing. Then they were used for transfection into HEK293LTV cells following the protocol described in Example 3. In this study, the plasmid, Vector EKQ with EGFP gene was used as transfection control, and the plasmid, Vector CPE with the Fc4-CNP36 gene as protein expression control. To evaluate the transfection efficiency, cells transfected with Vector EKQ were observed under fluorescence microscope (Data not shown). Cell culture supernatant at 5 days post-transfection were collected and analyzed for protein expression. The fusion proteins were detected by the method described in the Example 3 using the mouse-anti-human Fc antibodies conjugated with HRP (Cat. #A01854-200, GenScript, Nanjing, China). Lane content: M. Pre-stain protein marker; 1. Vector GAM; 2. Vector GGE; 3. Vector GGG; 4. Vector GGQ; 5. Vector GKA; 6. Vector GKK; 7. Vector EKQ; 8. Vector CPE; 9. Non-transfected cells (FIG. 17).

The western blot results showed that the C3i fusion proteins were highly expressed by the plasmids, Vector GGE and Vector GGG, but weakly expressed in the plasmids, Vector GGQ, Vector GKA, and Vector GKK (FIG. 17). The results indicated that the promoter, Fc fragment or the GOI change did not contribute to the improvement of the C3i fusion protein. However, the recognition site change (EVQL->DK) of Vh leader sequences or placing Fc4 in the 5′ end of C3i fusion gene dramatically improved the gene expression. Of note, Fc4 fragment in the construct Vector GGG uses the same the recognition site (DK) as Vector GGE. Thus, in this study the recognition site of Vh leader sequences played an important role on the optimization of C3i fusion gene expression.

(DK)C3i-Fc-endostatin protein expression

C3i-Fc-endostatin (Vector GKA) fusion gene had low expression. Since the recognition site change of Vh leader sequences could enhance the C3i-Fc4-CNP36 fusion protein expression, Vector GKA, was also changed to DK in the new plasmid, Vector GKC. The C3i-related GOI sequence in the plasmid, Vector GKC is shown in the Table 19, and the encoded protein sequence in the Table 20.

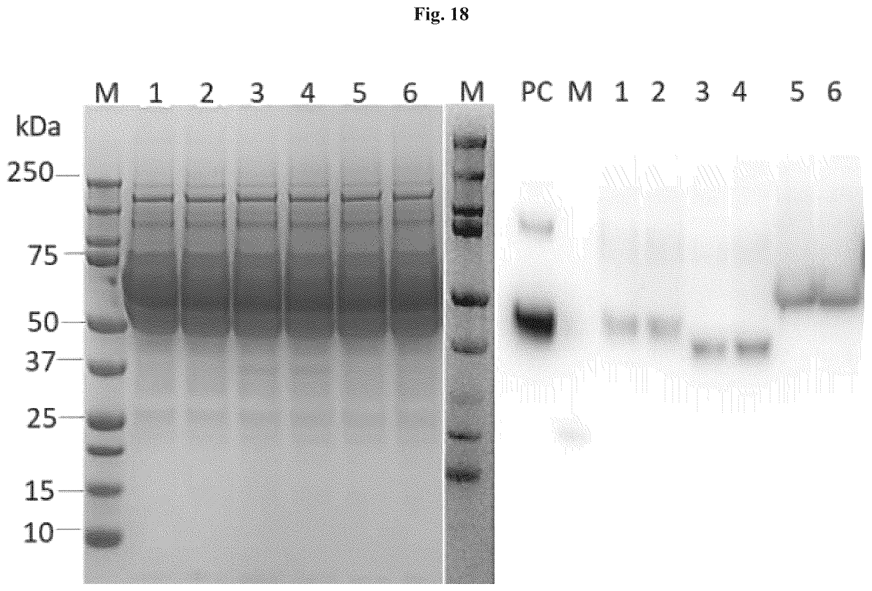

The protein expression by Vector GKC was not tested. Instead, a bacmid was prepared and the AAV was packaged and directly used for the protein expression test. FIG. 18 shows that the Vector GKC AAV had the similar C3i fusion protein expression level to the Vector GGE and Vector GGG AAVs, suggesting the recognition site alternation of Vh leader sequences also improved the C3i-Fc4-endostatin fusion gene expression. Lane content: M. Pre-stain protein marker; 1. Vector GGE (repeat 1); 2. Vector GGE (repeat 2); 3. Vector GGG (repeat 1); 4. Vector GGG (repeat 2); 5. Vector GKA (repeat 1); 6. Vector GKA (repeat 2); PC, positive control, purified Vector GGE proteins. In addition, when Vh leader sequences was used to drive the expression of a gene other than the C3i-related fusion protein (Data not shown), the recognition site, DK, also provided much high expression than EVQL. Therefore, in general, the recognition site optimization of a leader sequence was shown to be an important consideration regarding the gene expression improvement of a secretion protein.

TABLE 19
DNA sequences of plasmids used in Example 6

| Vector | | | SEQ ID |
|---|---|---|---|
| name | Protein | Nucleic acid sequence (ORF for GOI) | NO |
| Vector | C3i-Fc4- | ATGGAGTTCGGCCTGAGCTGGCTGTTCCTGGTGGCCATCC | 250 |
| GAM | CNP36 | TTAAGGGCGTGCAGTGCGAGGTGCAGCTGGGCATCTGCG | |
| | | TGTGGCAGGACTGGGGCGCCCACCGCTGCACCAACGGCG | |
| | | GAGGCGGAAGCGGCGGAGGCGGATCTGGCGGAGGCGGA | |
| | | AGCGACAAGAGAGTGGAGAGCAAGTACGGCCCCCCCTGC | |
| | | CCCCCCTGCCCCGCCCCCGAGTTCCTGGGCGGCCCCAGCG | |
| | | TGTTCCTGTTCCCCCCCAAGCCCAAGGACACCCTGATGAT | |
| | | CAGCAGAACCCCCGAGGTGACCTGCGTGGTGGTGGACGT | |
| | | GAGCCAGGAGGACCCCGAGGTGCAGTTCAACTGGTACGT | |
| | | GGACGGCGTGGAGGTGCACAACGCCAAGACCAAGCCCA | |
| | | GAGAGGAGCAGTTCAACAGCACCTACAGAGTGGTGAGCG | |
| | | TGCTGACCGTGCTGCACCAGGACTGGCTGAACGGCAAGG | |
| | | AGTACAAGTGCAAGGTGAGCAACAAGGGCCTGCCCAGCA | |
| | | GCATCGAGAAGACCATCAGCAAGGCCAAGGGCCAGCCCA | |
| | | GAGAGCCCCAGGTGTACACCCTGCCCCCCAGCCAGGAGG | |
| | | AGATGACCAAGAACCAGGTGAGCCTGACCTGCCTGGTGA | |
| | | AGGGCTTCTACCCCAGCGACATCGCCGTGGAGTGGGAGA | |
| | | GCAACGGCCAGCCCGAGAACAACTACAAGACCACCCCCC | |
| | | CCGTGCTGGACAGCGACGGCAGCTTCTTCCTGTACAGCA | |
| | | GACTGACCGTGGACAAGAGCAGATGGCAGGAGGGCAAC | |
| | | GTGTTCAGCTGCAGCGTGATGCACGAGGCCCTGCACAAC | |
| | | CACTACACCCAGAAGAGCCTGAGCCTGAGCCTGGGCAAG | |
| | | GGCGGAGGCGGAAGCGGCGGAGGCGGATCTGGCGGAGG | |
| | | CGGAAGCGGCGGAGGCGGCTCTGAGCACCCCAACGCGCG | |
| | | CAAATACAAAGGAGCCAACAAGAAGGGCTTGTCCAAGG | |
| | | GCTGCTTCGGCCTCAAGCTGGACCGAATCGGCTCCATGA | |
| | | GCGGCCTGGGATGTTAA | |
| Vector | EGFP | ATGGTGAGCAAGGGCGAGGAGCTGTTCACCGGGGTGGTG | 251 |
| EKQ | | CCCATCCTGGTCGAGCTGGACGGCGACGTAAACGGCCAC | |
| | | AAGTTCAGCGTGTCCGGCGAGGGCGAGGGCGATGCCACC | |
| | | TACGGCAAGCTGACCCTGAAGTTCATCTGCACCACCGGC | |
| | | AAGCTGCCCGTGCCCTGGCCCACCCTCGTGACCACCCTGA | |
| | | CCTACGGCGTGCAGTGCTTCAGCCGCTACCCCGACCACAT | |
| | | GAAGCAGCACGACTTCTTCAAGTCCGCCATGCCCGAAGG | |
| | | CTACGTCCAGGAGCGCACCATCTTCTTCAAGGACGACGG | |
| | | CAACTACAAGACCCGCGCCGAGGTGAAGTTCGAGGGCGA | |
| | | CACCCTGGTGAACCGCATCGAGCTGAAGGGCATCGACTT | |
| | | CAAGGAGGACGGCAACATCCTGGGGCACAAGCTGGAGTA | |
| | | CAACTACAACAGCCACAACGTCTATATCATGGCCGACAA | |
| | | GCAGAAGAACGGCATCAAGGTGAACTTCAAGATCCGCCA | |
| | | CAACATCGAGGACGGCAGCGTGCAGCTCGCCGACCACTA | |
| | | CCAGCAGAACACCCCCATCGGCGACGGCCCCGTGCTGCT | |
| | | GCCCGACAACCACTACCTGAGCACCCAGTCCGCCCTGAG | |
| | | CAAAGACCCCAACGAGAAGCGCGATCACATGGTCCTGCT | |
| | | GGAGTTCGTGACCGCCGCCGGGATCACTCTCGGCATGGA | |
| | | CGAGCTGTACAAGTAA | |
| Vector | Fc4-CNP36 | ATGGAGTTCGGCCTGAGCTGGCTGTTCCTGGTGGCCATCC | 252 |
| CPE | | TTAAGGGCGTGCAGTGCGACAAGAGAGTGGAGAGCAAGT | |
| | | ACGGCCCCCCCTGCCCCCCCTGCCCCGCCCCCGAGTTCCT | |
| | | GGGCGGCCCCAGCGTGTTCCTGTTCCCCCCCAAGCCCAAG | |
| | | GACACCCTGATGATCAGCAGAACCCCCGAGGTGACCTGC | |
| | | GTGGTGGTGGACGTGAGCCAGGAGGACCCCGAGGTGCAG | |
| | | TTCAACTGGTACGTGGACGGCGTGGAGGTGCACAACGCC | |
| | | AAGACCAAGCCCAGAGAGGAGCAGTTCAACAGCACCTAC | |
| | | AGAGTGGTGAGCGTGCTGACCGTGCTGCACCAGGACTGG | |
| | | CTGAACGGCAAGGAGTACAAGTGCAAGGTGAGCAACAA | |
| | | GGGCCTGCCCAGCAGCATCGAGAAGACCATCAGCAAGGC | |
| | | CAAGGGCCAGCCCAGAGAGCCCCAGGTGTACACCCTGCC | |
| | | CCCCAGCCAGGAGGAGATGACCAAGAACCAGGTGAGCCT | |
| | | GACCTGCCTGGTGAAGGGCTTCTACCCCAGCGACATCGC | |
| | | CGTGGAGTGGGAGAGCAACGGCCAGCCCGAGAACAACT | |
| | | ACAAGACCACCCCCCCCGTGCTGGACAGCGACGGCAGCT | |
| | | TCTTCCTGTACAGCAGACTGACCGTGGACAAGAGCAGAT | |
| | | GGCAGGAGGGCAACGTGTTCAGCTGCAGCGTGATGCACG | |
| | | AGGCCCTGCACAACCACTACACCCAGAAGAGCCTGAGCC | |
| | | TGAGCCTGGGCAAGGGCGGAGGCGGAAGCGGCGGAGGC | |
| | | GGATCTGGCGGAGGCGGAAGCGGCGGAGGCGGCTCTGA | |
| | | GCACCCCAACGCGCGCAAATACAAAGGAGCCAACAAGA | |
| | | AGGGCTTGTCCAAGGGCTGCTTCGGCCTCAAGCTGGACC | |
| | | GAATCGGCTCCATGAGCGGCCTGGGATGTTAA | |
| Vector | C3i-Fc4- | ATGGAGTTCGGCCTGAGCTGGCTGTTCCTGGTGGCCATCC | 253 |
| GGQ | CNP36 | TTAAGGGCGTGCAGTGCGAGGTGCAGCTGGGCATCTGCG | |
| | | TGTGGCAGGACTGGGGCGCCCACCGCTGCACCAACGGCG | |
| | | GAGGCGGAAGCGGCGGAGGCGGATCTGGCGGAGGCGGA | |
| | | AGCGACAAGAGAGTGGAGAGCAAGTACGGCCCCCCCTGC | |
| | | CCCCCCTGCCCCGCCCCCGAGTTCCTGGGCGGCCCCAGCG | |
| | | TGTTCCTGTTCCCCCCCAAGCCCAAGGACACCCTGATGAT | |
| | | CAGCAGAACCCCCGAGGTGACCTGCGTGGTGGTGGACGT | |
| | | GAGCCAGGAGGACCCCGAGGTGCAGTTCAACTGGTACGT | |
| | | GGACGGCGTGGAGGTGCACAACGCCAAGACCAAGCCCA | |
| | | GAGAGGAGCAGTTCAACAGCACCTACAGAGTGGTGAGCG | |
| | | TGCTGACCGTGCTGCACCAGGACTGGCTGAACGGCAAGG | |
| | | AGTACAAGTGCAAGGTGAGCAACAAGGGCCTGCCCAGCA | |
| | | GCATCGAGAAGACCATCAGCAAGGCCAAGGGCCAGCCCA | |
| | | GAGAGCCCCAGGTGTACACCCTGCCCCCCAGCCAGGAGG | |
| | | AGATGACCAAGAACCAGGTGAGCCTGACCTGCCTGGTGA | |
| | | AGGGCTTCTACCCCAGCGACATCGCCGTGGAGTGGGAGA | |
| | | GCAACGGCCAGCCCGAGAACAACTACAAGACCACCCCCC | |
| | | CCGTGCTGGACAGCGACGGCAGCTTCTTCCTGTACAGCA | |
| | | GACTGACCGTGGACAAGAGCAGATGGCAGGAGGGCAAC | |
| | | GTGTTCAGCTGCAGCGTGATGCACGAGGCCCTGCACAAC | |
| | | CACTACACCCAGAAGAGCCTGAGCCTGAGCCTGGGCAAG | |
| | | GGCGGAGGCGGAAGCGGCGGAGGCGGATCTGGCGGAGG | |
| | | CGGAAGCGGCGGAGGCGGCTCTGAGCACCCCAACGCGCG | |
| | | CAAATACAAAGGAGCCAACAAGAAGGGCTTGTCCAAGG | |
| | | GCTGCTTCGGCCTCAAGCTGGACCGAATCGGCTCCATGA | |
| | | GCGGCCTGGGATGTTAA | |
| Vector | (DK)C3i-Fc4- | ATGGAGTTCGGCCTGAGCTGGCTGTTCCTGGTGGCCATCC | 254 |
| GGE | CNP36 | TTAAGGGCGTGCAGTGCGACAAGGGCATCTGCGTGTGGC | |
| | | AGGACTGGGGCGCCCACCGCTGCACCAACGGCGGAGGCG | |
| | | GAAGCGGCGGAGGCGGATCTGGCGGAGGCGGAAGCGAC | |
| | | AAGAGAGTGGAGAGCAAGTACGGCCCCCCCTGCCCCCCC | |
| | | TGCCCCGCCCCCGAGTTCCTGGGCGGCCCCAGCGTGTTCC | |
| | | TGTTCCCCCCCAAGCCCAAGGACACCCTGATGATCAGCA | |
| | | GAACCCCCGAGGTGACCTGCGTGGTGGTGGACGTGAGCC | |
| | | AGGAGGACCCCGAGGTGCAGTTCAACTGGTACGTGGACG | |
| | | GCGTGGAGGTGCACAACGCCAAGACCAAGCCCAGAGAG | |
| | | GAGCAGTTCAACAGCACCTACAGAGTGGTGAGCGTGCTG | |
| | | ACCGTGCTGCACCAGGACTGGCTGAACGGCAAGGAGTAC | |
| | | AAGTGCAAGGTGAGCAACAAGGGCCTGCCCAGCAGCATC | |
| | | GAGAAGACCATCAGCAAGGCCAAGGGCCAGCCCAGAGA | |
| | | GCCCCAGGTGTACACCCTGCCCCCCAGCCAGGAGGAGAT | |
| | | GACCAAGAACCAGGTGAGCCTGACCTGCCTGGTGAAGGG | |
| | | CTTCTACCCCAGCGACATCGCCGTGGAGTGGGAGAGCAA | |
| | | CGGCCAGCCCGAGAACAACTACAAGACCACCCCCCCCGT | |
| | | GCTGGACAGCGACGGCAGCTTCTTCCTGTACAGCAGACT | |
| | | GACCGTGGACAAGAGCAGATGGCAGGAGGGCAACGTGTT | |
| | | CAGCTGCAGCGTGATGCACGAGGCCCTGCACAACCACTA | |
| | | CACCCAGAAGAGCCTGAGCCTGAGCCTGGGCAAGGGCGG | |
| | | AGGCGGAAGCGGCGGAGGCGGATCTGGCGGAGGCGGAA | |
| | | GCGGCGGAGGCGGCTCTGAGCACCCCAACGCGCGCAAAT | |
| | | ACAAAGGAGCCAACAAGAAGGGCTTGTCCAAGGGCTGCT | |
| | | TCGGCCTCAAGCTGGACCGAATCGGCTCCATGAGCGGCC | |
| | | TGGGATGTTAA | |
| Vector | Fc4-C3i | ATGGAGTTCGGCCTGAGCTGGCTGTTCCTGGTGGCCATCC | 255 |
| GGG | | TTAAGGGCGTGCAGTGCGACAAGAGAGTGGAGAGCAAGT | |
| | | ACGGCCCCCCCTGCCCCCCCTGCCCCGCCCCCGAGTTCCT | |
| | | GGGCGGCCCCAGCGTGTTCCTGTTCCCCCCCAAGCCCAAG | |
| | | GACACCCTGATGATCAGCAGAACCCCCGAGGTGACCTGC | |
| | | GTGGTGGTGGACGTGAGCCAGGAGGACCCCGAGGTGCAG | |
| | | TTCAACTGGTACGTGGACGGCGTGGAGGTGCACAACGCC | |
| | | AAGACCAAGCCCAGAGAGGAGCAGTTCAACAGCACCTAC | |
| | | AGAGTGGTGAGCGTGCTGACCGTGCTGCACCAGGACTGG | |
| | | CTGAACGGCAAGGAGTACAAGTGCAAGGTGAGCAACAA | |
| | | GGGCCTGCCCAGCAGCATCGAGAAGACCATCAGCAAGGC | |
| | | CAAGGGCCAGCCCAGAGAGCCCCAGGTGTACACCCTGCC | |
| | | CCCCAGCCAGGAGGAGATGACCAAGAACCAGGTGAGCCT | |
| | | GACCTGCCTGGTGAAGGGCTTCTACCCCAGCGACATCGC | |
| | | CGTGGAGTGGGAGAGCAACGGCCAGCCCGAGAACAACT | |
| | | ACAAGACCACCCCCCCCGTGCTGGACAGCGACGGCAGCT | |
| | | TCTTCCTGTACAGCAGACTGACCGTGGACAAGAGCAGAT | |
| | | GGCAGGAGGGCAACGTGTTCAGCTGCAGCGTGATGCACG | |
| | | AGGCCCTGCACAACCACTACACCCAGAAGAGCCTGAGCC | |
| | | TGAGCCTGGGCAAGGGCGGAGGCGGAAGCGGCGGAGGC | |
| | | GGATCTGGCGGAGGCGGAAGCGGCGGAGGCGGCTCTGGC | |
| | | ATCTGCGTGTGGCAGGACTGGGGCGCCCACCGCTGCACC | |
| | | AACTAA | |
| Vector | Aflibercept- | ATGGTGAGCTACTGGGACACCGGCGTGCTGCTGTGCGCC | 256 |
| GKK | Fc1-C3i | CTGCTGAGCTGCCTGCTGCTGACCGGCAGCAGCAGCGGC | |
| | | AGCGACACCGGCAGGCCCTTCGTGGAGATGTACTCCGAG | |
| | | ATCCCCGAGATCATCCACATGACCGAGGGCAGGGAGCTG | |
| | | GTGATCCCCTGCAGGGTGACCTCCCCCAACATCACCGTGA | |
| | | CCCTGAAGAAGTTCCCCCTGGACACCCTGATCCCCGACG | |
| | | GCAAGAGGATCATCTGGGACTCCAGGAAGGGCTTCATCA | |
| | | TCTCCAACGCCACCTACAAGGAGATCGGCCTGCTGACCT | |
| | | GCGAGGCCACCGTGAACGGCCACCTGTACAAGACCAACT | |
| | | ACCTGACCCACAGGCAGACCAACACCATCATCGACGTGG | |
| | | TGCTGTCCCCCTCCCACGGCATCGAGCTGTCCGTGGGCGA | |
| | | GAAGCTGGTGCTGAACTGCACCGCCAGGACCGAGCTGAA | |
| | | CGTGGGCATCGACTTCAACTGGGAGTACCCCTCCTCCAAG | |
| | | CACCAGCACAAGAAGCTGGTGAACAGGGACCTGAAGACC | |
| | | CAGTCCGGCTCCGAGATGAAGAAGTTCCTGTCCACCCTG | |
| | | ACCATCGACGGCGTGACCAGGTCCGACCAGGGCCTGTAC | |
| | | ACCTGCGCCGCCTCCTCCGGCCTGATGACCAAGAAGAAC | |
| | | TCCACCTTCGTGAGGGTGCACGAGAAGGACAAGACCCAC | |
| | | ACCTGCCCCCCCTGCCCCGCCCCCGAGCTGCTGGGCGGCC | |
| | | CCTCCGTGTTCCTGTTCCCCCCCAAGCCCAAGGACACCCT | |
| | | GATGATCTCCAGGACCCCCGAGGTGACCTGCGTGGTGGT | |
| | | GGACGTGTCCCACGAGGACCCCGAGGTGAAGTTCAACTG | |
| | | GTACGTGGACGGCGTGGAGGTGCACAACGCCAAGACCAA | |
| | | GCCCAGGGAGGAGCAGTACAACTCCACCTACAGGGTGGT | |
| | | GTCCGTGCTGACCGTGCTGCACCAGGACTGGCTGAACGG | |
| | | CAAGGAGTACAAGTGCAAGGTGTCCAACAAGGCCCTGCC | |
| | | CGCCCCCATCGAGAAGACCATCTCCAAGGCCAAGGGCCA | |
| | | GCCCAGGGAGCCCCAGGTGTACACCCTGCCCCCCTCCAG | |
| | | GGACGAGCTGACCAAGAACCAGGTGTCCCTGACCTGCCT | |
| | | GGTGAAGGGCTTCTACCCCTCCGACATCGCCGTGGAGTG | |
| | | GGAGTCCAACGGCCAGCCCGAGAACAACTACAAGACCAC | |
| | | CCCCCCCGTGCTGGACTCCGACGGCTCCTTCTTCCTGTAC | |
| | | TCCAAGCTGACCGTGGACAAGTCCAGGTGGCAGCAGGGC | |
| | | AACGTGTTCTCCTGCTCCGTGATGCACGAGGCCCTGCACA | |
| | | ACCACTACACCCAGAAGTCCCTGTCCCTGTCCCCCGGCAA | |
| | | GGGCGGAGGCGGAAGCGGCGGAGGCGGATCTGGCGGAG | |
| | | GCGGAAGCGGCGGAGGCGGCTCTGGCATCTGCGTGTGGC | |
| | | AGGACTGGGGCGCCCACCGCTGCACCAACTAA | |
| Vector | C3i-Fc4- | ATGGAGTTCGGCCTGAGCTGGCTGTTCCTGGTGGCCATCC | 257 |
| GKA | endostatin | TTAAGGGCGTGCAGTGCGAGGTGCAGCTGGGCATCTGCG | |
| | | TGTGGCAGGACTGGGGCGCCCACCGCTGCACCAACGGCG | |
| | | GAGGCGGAAGCGGCGGAGGCGGATCTGGCGGAGGCGGA | |
| | | AGCGACAAGAGAGTGGAGAGCAAGTACGGCCCCCCCTGC | |
| | | CCCCCCTGCCCCGCCCCCGAGTTCCTGGGCGGCCCCAGCG | |
| | | TGTTCCTGTTCCCCCCCAAGCCCAAGGACACCCTGATGAT | |
| | | CAGCAGAACCCCCGAGGTGACCTGCGTGGTGGTGGACGT | |
| | | GAGCCAGGAGGACCCCGAGGTGCAGTTCAACTGGTACGT | |
| | | GGACGGCGTGGAGGTGCACAACGCCAAGACCAAGCCCA | |
| | | GAGAGGAGCAGTTCAACAGCACCTACAGAGTGGTGAGCG | |
| | | TGCTGACCGTGCTGCACCAGGACTGGCTGAACGGCAAGG | |
| | | AGTACAAGTGCAAGGTGAGCAACAAGGGCCTGCCCAGCA | |
| | | GCATCGAGAAGACCATCAGCAAGGCCAAGGGCCAGCCCA | |
| | | GAGAGCCCCAGGTGTACACCCTGCCCCCCAGCCAGGAGG | |
| | | AGATGACCAAGAACCAGGTGAGCCTGACCTGCCTGGTGA | |
| | | AGGGCTTCTACCCCAGCGACATCGCCGTGGAGTGGGAGA | |
| | | GCAACGGCCAGCCCGAGAACAACTACAAGACCACCCCCC | |
| | | CCGTGCTGGACAGCGACGGCAGCTTCTTCCTGTACAGCA | |
| | | GACTGACCGTGGACAAGAGCAGATGGCAGGAGGGCAAC | |
| | | GTGTTCAGCTGCAGCGTGATGCACGAGGCCCTGCACAAC | |
| | | CACTACACCCAGAAGAGCCTGAGCCTGAGCCTGGGCAAG | |
| | | GGCGGAGGCGGAAGCGGCGGAGGCGGATCTGGCGGAGG | |
| | | CGGAAGCGGCGGAGGCGGCTCTCACAGCCACAGGGACTT | |
| | | CCAGCCTGTGCTGCACCTGGTGGCCCTGAACAGCCCTCTG | |
| | | AGCGGAGGAATGAGGGGAATCAGGGGAGCCGACTTCCA | |
| | | GTGCTTCCAGCAGGCCAGGGCCGTGGGACTGGCCGGAAC | |
| | | CTTCAGGGCCTTCCTGAGCAGCAGGCTGCAGGACCTGTA | |
| | | CAGCATCGTGAGGAGGGCCGACAGGGCCGCCGTGCCCAT | |
| | | CGTGAACCTGAAGGACGAGCTGCTGTTCCCTAGCTGGGA | |
| | | GGCCCTGTTCAGCGGAAGCGAGGGACCCCTGAAGCCCGG | |
| | | AGCCAGGATCTTCAGCTTCGACGGAAAGGACGTGCTGAG | |
| | | GCACCCTACCTGGCCTCAGAAGAGCGTGTGGCACGGAAG | |
| | | CGACCCTAACGGAAGGAGGCTGACCGAGAGCTACTGCGA | |
| | | GACCTGGAGGACCGAGGCCCCTAGCGCCACCGGCCAGGC | |
| | | CAGCAGCCTGCTGGGAGGAAGGCTGCTGGGACAGAGCGC | |
| | | CGCCAGCTGCCACCACGCCTACATCGTGCTGTGCATCGAG | |
| | | AACAGCTTCATGACCGCCAGCAAGTGA | |
| Vector | (DK)C3i- | ATGGAGTTCGGCCTGAGCTGGCTGTTCCTGGTGGCCATCC | 258 |
| GKC | Fc4- | TTAAGGGCGTGCAGTGCGACAAGGGCATCTGCGTGTGGC | |
| | endostatin | AGGACTGGGGCGCCCACCGCTGCACCAACGGCGGAGGCG | |
| | | GAAGCGGCGGAGGCGGATCTGGCGGAGGCGGAAGCGAC | |
| | | AAGAGAGTGGAGAGCAAGTACGGCCCCCCCTGCCCCCCC | |
| | | TGCCCCGCCCCCGAGTTCCTGGGCGGCCCCAGCGTGTTCC | |
| | | TGTTCCCCCCCAAGCCCAAGGACACCCTGATGATCAGCA | |
| | | GAACCCCCGAGGTGACCTGCGTGGTGGTGGACGTGAGCC | |
| | | AGGAGGACCCCGAGGTGCAGTTCAACTGGTACGTGGACG | |
| | | GCGTGGAGGTGCACAACGCCAAGACCAAGCCCAGAGAG | |
| | | GAGCAGTTCAACAGCACCTACAGAGTGGTGAGCGTGCTG | |
| | | ACCGTGCTGCACCAGGACTGGCTGAACGGCAAGGAGTAC | |
| | | AAGTGCAAGGTGAGCAACAAGGGCCTGCCCAGCAGCATC | |
| | | GAGAAGACCATCAGCAAGGCCAAGGGCCAGCCCAGAGA | |
| | | GCCCCAGGTGTACACCCTGCCCCCCAGCCAGGAGGAGAT | |
| | | GACCAAGAACCAGGTGAGCCTGACCTGCCTGGTGAAGGG | |
| | | CTTCTACCCCAGCGACATCGCCGTGGAGTGGGAGAGCAA | |
| | | CGGCCAGCCCGAGAACAACTACAAGACCACCCCCCCCGT | |
| | | GCTGGACAGCGACGGCAGCTTCTTCCTGTACAGCAGACT | |
| | | GACCGTGGACAAGAGCAGATGGCAGGAGGGCAACGTGTT | |
| | | CAGCTGCAGCGTGATGCACGAGGCCCTGCACAACCACTA | |
| | | CACCCAGAAGAGCCTGAGCCTGAGCCTGGGCAAGGGCGG | |
| | | AGGCGGAAGCGGCGGAGGCGGATCTGGCGGAGGCGGAA | |
| | | GCGGCGGAGGCGGCTCTCACAGCCACAGGGACTTCCAGC | |
| | | CTGTGCTGCACCTGGTGGCCCTGAACAGCCCTCTGAGCGG | |
| | | AGGAATGAGGGGAATCAGGGGAGCCGACTTCCAGTGCTT | |
| | | CCAGCAGGCCAGGGCCGTGGGACTGGCCGGAACCTTCAG | |
| | | GGCCTTCCTGAGCAGCAGGCTGCAGGACCTGTACAGCAT | |
| | | CGTGAGGAGGGCCGACAGGGCCGCCGTGCCCATCGTGAA | |
| | | CCTGAAGGACGAGCTGCTGTTCCCTAGCTGGGAGGCCCT | |
| | | GTTCAGCGGAAGCGAGGGACCCCTGAAGCCCGGAGCCAG | |
| | | GATCTTCAGCTTCGACGGAAAGGACGTGCTGAGGCACCC | |
| | | TACCTGGCCTCAGAAGAGCGTGTGGCACGGAAGCGACCC | |
| | | TAACGGAAGGAGGCTGACCGAGAGCTACTGCGAGACCTG | |
| | | GAGGACCGAGGCCCCTAGCGCCACCGGCCAGGCCAGCAG | |
| | | CCTGCTGGGAGGAAGGCTGCTGGGACAGAGCGCCGCCAG | |
| | | CTGCCACCACGCCTACATCGTGCTGTGCATCGAGAACAG | |
| | | CTTCATGACCGCCAGCAAGTGA | |

TABLE 20
Peptides and fusion protein sequences of Example 6

| Vector | | | |
|---|---|---|---|
| name | Protein | Nucleic acid sequence (ORF for GOI) | SEQ ID NO |
| Vector | C3i-Fc4- | MEFGLSWLFLVAILKGVQCEVQLGICVWQDWGAHRCTNG | |
| GAM | CNP36 | GGGSGGGGSGGGGSDKRVESKYGPPCPPCPAPEFLGGPSVF | |
| | (CFC36) | LFPPKPKDTLMISRTPEVTCVVVDVSQEDPEVQFNWYVDG | |
| | | VEVHNAKTKPREEQFNSTYRVVSVLTVLHQDWLNGKEYK | 277 |
| | | CKVSNKGLPSSIEKTISKAKGQPREPQVYTLPPSQEEMTKN | |
| | | QVSLTCLVKGFYPSDIAVEWESNGQPENNYKTTPPVLDSD | |
| | | GSFFLYSRLTVDKSRWQEGNVFSCSVMHEALHNHYTQKSL | |
| | | SLSLGKGGGGSGGGGSGGGGSGGGGSEHPNARKYKGANK | |
| | | KGLSKGCFGLKLDRIGSMSGLGC | |
| Vector | EGFP | MVSKGEELFTGVVPILVELDGDVNGHKFSVSGEGEGDATY | 278 |
| EKQ | | GKLTLKFICTTGKLPVPWPTLVTTLTYGVQCFSRYPDHMK | |
| | | QHDFFKSAMPEGYVQERTIFFKDDGNYKTRAEVKFEGDTL | |
| | | VNRIELKGIDFKEDGNILGHKLEYNYNSHNVYIMADKQKN | |
| | | GIKVNFKIRHNIEDGSVQLADHYQQNTPIGDGPVLLPDNHY | |
| | | LSTQSALSKDPNEKRDHMVLLEFVTAAGITLGMDELYK | |
| Vector | Fc4- | MEFGLSWLFLVAILKGVQCDKRVESKYGPPCPPCPAPEFLG | 279 |
| CPE | (G4S)4- | GPSVFLFPPKPKDTLMISRTPEVTCVVVDVSQEDPEVQFNW | |
| | CNP36 | YVDGVEVHNAKTKPREEQFNSTYRVVSVLTVLHQDWLNG | |
| | | KEYKCKVSNKGLPSSIEKTISKAKGQPREPQVYTLPPSQEE | |
| | | MTKNQVSLTCLVKGFYPSDIAVEWESNGQPENNYKTTPPV | |
| | | LDSDGSFFLYSRLTVDKSRWQEGNVFSCSVMHEALHNHYT | |
| | | QKSLSLSLGKGGGGSGGGGSGGGGSGGGGSEHPNARKYK | |
| | | GANKKGLSKGCFGLKLDRIGSMSGLGC | |
| Vector | C3i-Fc4- | MEFGLSWLFLVAILKGVQCEVQLGICVWQDWGAHRCTNG | 280 |
| GGQ | CNP36 | GGGSGGGGSGGGGSDKRVESKYGPPCPPCPAPEFLGGPSVF | |
| | | LFPPKPKDTLMISRTPEVTCVVVDVSQEDPEVQFNWYVDG | |
| | | VEVHNAKTKPREEQFNSTYRVVSVLTVLHQDWLNGKEYK | |
| | | CKVSNKGLPSSIEKTISKAKGQPREPQVYTLPPSQEEMTKN | |
| | | QVSLTCLVKGFYPSDIAVEWESNGQPENNYKTTPPVLDSD | |
| | | GSFFLYSRLTVDKSRWQEGNVFSCSVMHEALHNHYTQKSL | |
| | | SLSLGKGGGGSGGGGSGGGGSGGGGSEHPNARKYKGANK | |
| | | KGLSKGCFGLKLDRIGSMSGLGC | |
| Vector | (DK)C3i- | MEFGLSWLFLVAILKGVQCDKGICVWQDWGAHRCTNGGG | 281 |
| GGE | Fc4- | GSGGGGSGGGGSDKRVESKYGPPCPPCPAPEFLGGPSVFLF | |
| | CNP36 | PPKPKDTLMISRTPEVTCVVVDVSQEDPEVQFNWYVDGVE | |
| | | VHNAKTKPREEQFNSTYRVVSVLTVLHQDWLNGKEYKCK | |
| | | VSNKGLPSSIEKTISKAKGQPREPQVYTLPPSQEEMTKNQV | |
| | | SLTCLVKGFYPSDIAVEWESNGQPENNYKTTPPVLDSDGSF | |
| | | FLYSRLTVDKSRWQEGNVFSCSVMHEALHNHYTQKSLSLS | |
| | | LGKGGGGSGGGGSGGGGSGGGGSEHPNARKYKGANKKG | |
| | | LSKGCFGLKLDRIGSMSGLGC | |
| Vector | Fc4-C3i | MEFGLSWLFLVAILKGVQCDKRVESKYGPPCPPCPAPEFLG | 282 |
| GGG | | GPSVFLFPPKPKDTLMISRTPEVTCVVVDVSQEDPEVQFNW | |
| | | YVDGVEVHNAKTKPREEQFNSTYRVVSVLTVLHQDWLNG | |
| | | KEYKCKVSNKGLPSSIEKTISKAKGQPREPQVYTLPPSQEE | |
| | | MTKNQVSLTCLVKGFYPSDIAVEWESNGQPENNYKTTPPV | |
| | | LDSDGSFFLYSRLTVDKSRWQEGNVFSCSVMHEALHNHYT | |
| | | QKSLSLSLGKGGGGSGGGGSGGGGSGGGGSGICVWQDWG | |
| | | AHRCTN | |
| Vector | Afliberce | MVSYWDTGVLLCALLSCLLLTGSSSGSDTGRPFVEMYSEIP | 283 |
| GKK | pt-Fc1- | EIIHMTEGRELVIPCRVTSPNITVTLKKFPLDTLIPDGKRIIW | |
| | C3i | DSRKGFIISNATYKEIGLLTCEATVNGHLYKTNYLTHRQTN | |
| | | TIIDVVLSPSHGIELSVGEKLVLNCTARTELNVGIDENWEYP | |
| | | SSKHQHKKLVNRDLKTQSGSEMKKFLSTLTIDGVTRSDQG | |
| | | LYTCAASSGLMTKKNSTFVRVHEKDKTHTCPPCPAPELLG | |
| | | GPSVFLFPPKPKDTLMISRTPEVTCVVVDVSHEDPEVKFNW | |
| | | YVDGVEVHNAKTKPREEQYNSTYRVVSVLTVLHQDWLNG | |
| | | KEYKCKVSNKALPAPIEKTISKAKGQPREPQVYTLPPSRDE | |
| | | LTKNQVSLTCLVKGFYPSDIAVEWESNGQPENNYKTTPPVL | |
| | | DSDGSFFLYSKLTVDKSRWQQGNVFSCSVMHEALHNHYT | |
| | | QKSLSLSPGKGGGGSGGGGSGGGGSGGGGSGICVWQDWG | |
| | | AHRCTN | |
| Vector | C3i-Fc4- | MEFGLSWLFLVAILKGVQCEVQLGICVWQDWGAHRCTNG | 284 |
| GKA | endostati | GGGSGGGGSGGGGSDKRVESKYGPPCPPCPAPEFLGGPSVF | |
| | n | LFPPKPKDTLMISRTPEVTCVVVDVSQEDPEVQFNWYVDG | |
| | | VEVHNAKTKPREEQFNSTYRVVSVLTVLHQDWLNGKEYK | |
| | | CKVSNKGLPSSIEKTISKAKGQPREPQVYTLPPSQEEMTKN | |
| | | QVSLTCLVKGFYPSDIAVEWESNGQPENNYKTTPPVLDSD | |
| | | GSFFLYSRLTVDKSRWQEGNVFSCSVMHEALHNHYTQKSL | |
| | | SLSLGKGGGGSGGGGSGGGGSGGGGSHSHRDFQPVLHLV | |
| | | ALNSPLSGGMRGIRGADFQCFQQARAVGLAGTFRAFLSSR | |
| | | LQDLYSIVRRADRAAVPIVNLKDELLFPSWEALFSGSEGPL | |
| | | KPGARIFSFDGKDVLRHPTWPQKSVWHGSDPNGRRLTESY | |
| | | CETWRTEAPSATGQASSLLGGRLLGQSAASCHHAYIVLCIE | |
| | | NSFMTASK | |
| Vector | (DK) | MEFGLSWLFLVAILKGVQCDKGICVWQDWGAHRCTNGGG | |
| GKC | C3i-Fc4- | GSGGGGSGGGGSDKRVESKYGPPCPPCPAPEFLGGPSVFLF | |
| | endostatin | PPKPKDTLMISRTPEVTCVVVDVSQEDPEVQFNWYVDGVE | |
| | | VHNAKTKPREEQFNSTYRVVSVLTVLHQDWLNGKEYKCK | |
| | | VSNKGLPSSIEKTISKAKGQPREPQVYTLPPSQEEMTKNQV | |
| | | SLTCLVKGFYPSDIAVEWESNGQPENNYKTTPPVLDSDGSF | |
| | | FLYSRLTVDKSRWQEGNVFSCSVMHEALHNHYTQKSLSLS | 285 |
| | | LGKGGGGSGGGGSGGGGSGGGGSHSHRDFQPVLHLVALN | |
| | | SPLSGGMRGIRGADFQCFQQARAVGLAGTFRAFLSSRLQD | |
| | | LYSIVRRADRAAVPIVNLKDELLFPSWEALFSGSEGPLKPG | |
| | | ARIFSFDGKDVLRHPTWPQKSVWHGSDPNGRRLTESYCET | |
| | | WRTEAPSATGQASSLLGGRLLGQSAASCHHAYIVLCIENSF | |
| | | MTASK | |

### Example 7. C3i Fusion Protein Purification and its N-Terminal Sequencing

In the last experiment, Vector GGE and Vector GGG were shown to highly express C3i fusion genes. In this experiment, the adherent HEK293LTV cells were seeded in 6-well plates, and a large number of plasmids were prepared and transfected into the suspension Expi293F cells for large-scale protein purification. Expi293F cells were cultured in the BalanCD HEP293 medium (Cat, #91165, Fujifim, Tokyo, Japan) with 25 mM of L-glutamine (Cat, #35050-61, ThermoFisher Scientific, Waltham, MA, USA). 1-2 hours prior to transfection, cells were diluted at a density of 1×10⁶ cells/ml into a transfection culture vessel with 150 mL of culture medium. In a separate sterile tube, 600 μg of plasmid DNA and 15 mL of diluent were combined and vortexed briefly. 1200 μL of PEImax was added to the diluted DNA, and the mixture was vortexed for 5 min and incubated at room temperature for 20 minutes without additional agitation allowing transfection complex formation. After gently mixing the solution by pipetting up and down, the entire solution was added to 150 mL of suspension cell culture. The flasks were shaken in an incubator for 2-3 hours before adding 150 ml of fresh medium into each flask. The cell cultures were incubated for 5 days prior to harvest.

The C3i fusion proteins were detected in the SDS-PAGE gel. All of the cell culture supernatant was collected for the protein purification using protein A resin (Cat. #L00210, GenScript USA, Piscataway, NJ, USA). After completely resuspending the resin, 2 ml of resin was transferred into a new tube. The beads were spun down and the supernatant was discarded. 12 ml binding/wash buffer (150 mM NaCl, 20 mM Na₂HPO₄, pH 7.0) was added to the beads and vortexed for 20 seconds. The beads were spun down and the supernatant discarded again. Beads were diluted into binding/wash buffer (1:1) [300 ml+300 mL] and pH was adjusted to 7.0. Then the washed beads were added to the diluted sample and incubated at 4° C. overnight. The resin/diluted sample mixture was centrifuged and the supernatant was discarded. The resin with remaining supernatant was loaded onto a column and the flow through sample was collected for analysis. 90 mL of wash buffer was added to the resins in the column and the wash sample was collected for analysis. The wash process was repeated again. Finally, the protein was eluted with 3 mL of elution buffer (0.1 M Glycine, pH 3.0) 3 times. The elution was neutralized to pH 7.4 using 300 μL of neutralization buffer (1M Tris.Cl, pH8.5). Finally, the sample was concentrated using 3 kDa cutoff Amicon® Ultra Centrifugal Filter column (Cat. #UFC900308, Millipore-Sigma, Burlington, MA, USA), and buffer exchange was performed.

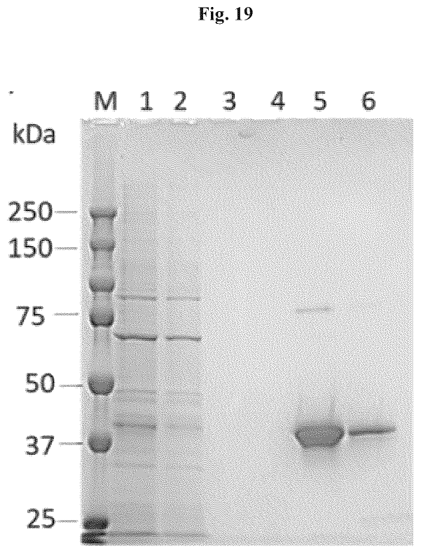

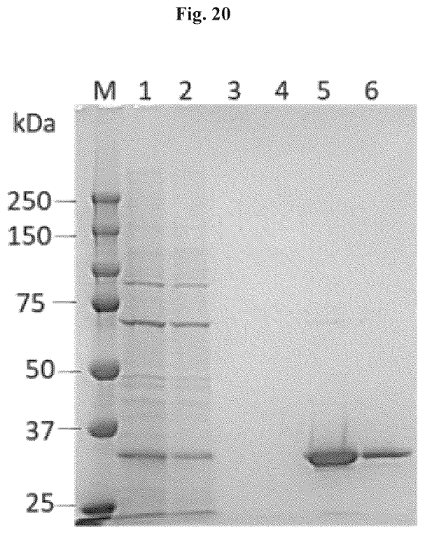

After the flow-through, wash, and elution samples of Vector GGE and Vector GGG were collected above, they were analyzed by SDS-PAGE gel. Most of Fc-fusion proteins were able to bind to protein A resins and were eluted by low pH of Glycine buffer. Both purified Vector GGE (FIG. 19) and Vector GGG (FIG. 20) proteins were of high purity and a dimer band was observed for each. Lane content for both gels: M. Protein marker; 1. cell culture medium; 2. flow through; 3. Wash 1; 4. Wash 2; 5. Elution sample; 6. Proteins in PBS buffer. The result showed that the Vector GGE and Vector GGG was very well purified and some dimer formed even in the reducing loading buffer.

After the elution buffer was exchanged with PBS buffer, the purified protein was quantified by BCA protein assay kit (Cat. #23225, Thermo Scientific, Waltham, MA, USA) and stored at ≤−60° C. until testing.

### C3i Fusion Protein N-Terminal Sequencing

To further identify the purified Vector GGE and Vector GGG proteins, the purified proteins were sent for protein N-terminal sequencing. The proteins were blotted onto a PVDF membrane and stained with SimplyBlue SafeStain (Cat. #465034, Thermo Fisher Scientific), the band of each sample containing approximately 8 ug proteins was individually cut and kept in the 5 mL of Eppendorf tube. The bands were thoroughly de-stained with 50%, 60%, and 70% of methanol for 1 min sequentially. Then the blots were washed by milliQ water for 15 min. Airdry the blot overnight.

The de-stained membrane is transferred to a fresh tube and stored at 4° C. until the samples were shipped on ice pack to Creative Proteomics (Shirley, NY, USA) for N-terminus first 5 amino acids identification.

The N-terminal sequencing results showed that the first 4 amino acids of Vector GGE (Table 43) and 5 amino acids of Vector GGG (Table 44) are identical to the expected sequences. Since the fifth amino acid was Cysteine for Vector GGE, it could not be identified by a regular method. These results demonstrated that both Vector GGE and Vector GGG proteins were correctly translated, cleaved from the signaling peptide, and secreted out of cells.

TABLE 43
Vector GGE protein N-terminal sequencing results

| Cycle | Residue | Amino Acid |
|---|---|---|
| 1 | Blank | — |
| 2 | Standard | — |
| 3 | 1 | D |
| 4 | 2 | K, (L, T, P) |
| 5 | 3 | G, (P, E, F) |
| 6 | 4 | I, (V, P) |
| 7 | 5 | (V, F, L, S) |

TABLE 44
Vector GGG protein N-terminal sequencing results

| Cycle | Residue | Amino Acid |
|---|---|---|
| 1 | Blank | — |
| 2 | Standard | — |
| 3 | 1 | D |
| 4 | 2 | K |
| 5 | 3 | V |
| 6 | 4 | E |
| 7 | 5 | (V, F, L, S) |

### Example 8. Optimization of C3i Fusion Protein Engineering

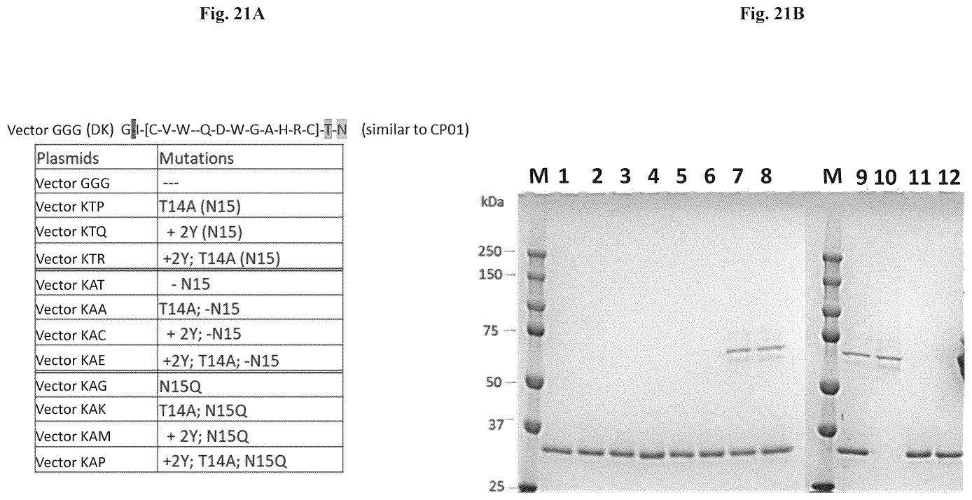

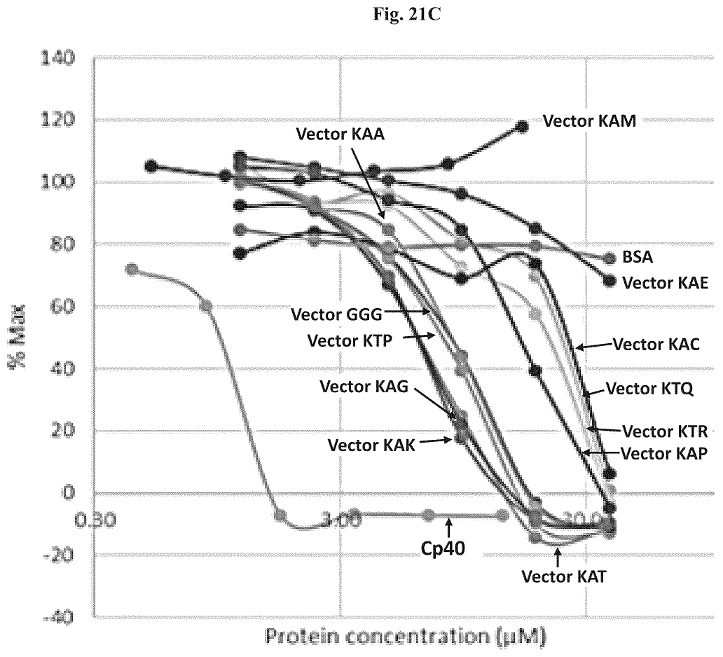

It was previously shown that when the Compstatin sequence (G-I-[C-V-W-Q-D-W-G-A-H-R-C]-T-N) was mutated with unnatured amino acids, it showed higher biding affinity to C3b than the wildtype Compstatin. Therefore, four mutations were designed, as shown in the FIG. 21A, and introduced into Vector GGG. Some designed mutants were insertion of Glycine (Y) in between 1^{st} and 2^{nd} amino acids (+2Y), mutation of Threonine (T) into Alanine (A) at the 14th amino acid (T14A), deletion of Asparagine (N) at 15th amino acid (−N15), and mutation of Asparagine (N) into Glutamine (Q) at 15th amino acid (N15Q). Various combination of these mutations resulted in 11 mutants, each of which carried 1, 2 or 3 different mutations.

The ORF sequences of GOI and their encoded protein sequences are listed in Table 26 and Table 27, respectively. New plasmids Vector KTP, Vector KTQ, Vector KTR, Vector KAP, Vector KAA, Vector KAC, Vector KAE, Vector KAG, Vector KAK, Vector KAM, and Vector KAP, were generated with the described mutations and were cloned and confirmed by Sanger sequencing. They were transfected into the suspension Expi293F cells following the protocol in Example 7. When all the mutant proteins were expressed and detected by SDS-PAGE gel (Data not shown), they were purified using protein A resins following the protocol in Example 7. FIG. 21B showed that all the mutant proteins were successfully purified with high purity from the culture medium. Lane content: M: Protein marker; 1, Vector KTP; 2, Vector KTQ; 3, Vector KTR; 4, Vector KAT; 5, Vector KAA; 6, Vector KAC; 7, Vector KAE; 8, Vector KAG; 9, Vector KAK; 10, Vector KAM; 11, Vector KAP; 12, Vector GGG. Among them, Vector KTP, Vector KTQ, Vector KTR, Vector KAP, Vector KAA, and Vector KAC, had only a single band with the size of about 29 kDa, and Vector KAE, Vector KAG, and Vector KAK had two bands, about 29 kDa and 58 kDa (suspected as a dimer). However, only Vector KAM had a single 58 kDa band, suggesting all the proteins form dimers even in the presence of reducing reagents. After the purified proteins were quantified by BCA, they were stored at ≤−60° C. for hemolysis inhibition assay.

In parallel, serum dose causing approximately 70% hemolysis was determined for new normal human serum (NHS) before the purified proteins were used for a hemolysis inhibition assay. Various doses of NHS were mixed with rabbit erythrocytes (Cat. B300, Complement Technology) in the presence of 0.1 M Mg2+ EGTA (Cat. B106, Complement Technology). Background lysis was measured using GVBE (GVBo plus 20 mM EDTA, pH 7.4), and maximum lysis was measured by lysing all the cells with Maximum Lysis Buffer (GVBo+0.2% NP40). After all the samples were mixed on ice, they were transferred to 37° C. water bath. The samples were vortexed every 5 minutes and incubated for 30 minutes in total. 1 mL of cold GVBE was added into each tube to stop the reaction. After samples were centrifuged for 3 min at approximately 1000× g to pellet cells, the supernatant was transferred to a cuvette and read at 412 nm in SpectaMax iD3 microplate reader. Each reading was normalized to the background, divided by Max Lysis minus background, and multiplied by 100, which equaled % of Max Lysis. Using the GraphPad Prism software, the % of Max Lysis was plotted against microliters of NHS, and the dose of NHS causing 50% and 70% hemolysis was respectively determined.

Using the NHS dose causing 70% hemolysis, the purified mutant proteins were utilized for a hemolysis inhibition assay. All mutant proteins were 2× serially diluted and mixed with the rabbit erythrocytes in the presence of NHS, resulting in the final concentration of 32.77, 16.38, 8.9, 4.10, 2.05, and 1.02 μM of each mutant protein. The benchmark, CP40 peptide, was also used in this study. After each reading was subtracted from the background, it was divided by 70% hemolysis minus background and multiplied by 100. The value was considered as % of hemolysis inhibition. The percentage of hemolysis inhibition was plotted against the molecular concentration of mutant proteins by GraphPad Prism software and IC50 value was estimated based on the data plotting.

Hemolysis inhibition assay results for all mutant proteins (Vector KTP, Vector KTQ, Vector KTR, Vector KAP, Vector KAA, Vector KAC, Vector KAE, Vector KAG, Vector KAK, Vector KAM, and Vector KAP) showed that, compared with parental protein, Vector GGG, the IC50 of proteins, Vector KAT, Vector KAG, and Vector KAK decreased, Vector KTP and Vector KAA remained the same, Vector KAC, Vector KAM, and Vector KAP significantly increased, and Vector KTQ, Vector KTR, and Vector KAE nearly lost hemolysis inhibition activities completely. BSA was used as a negative control and CP40 was included as a control (FIG. 21C, Table 21). Interestingly, IC50 alternation data indicate that either presence of Asparagine (N) or introduction of Tyrosine (Y) compromised the protein hemolytic inhibition activity and combination of both mutation caused the major loss of the protein hemolytic inhibition activity. Vector KAG mutation was selected for continued C3i fusion protein engineering.

### C3i Location Alternation

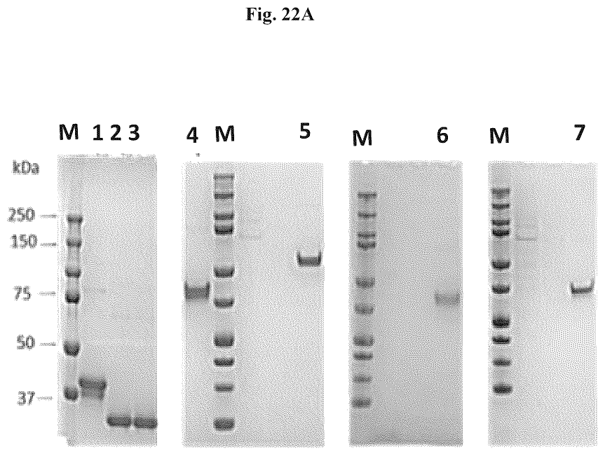

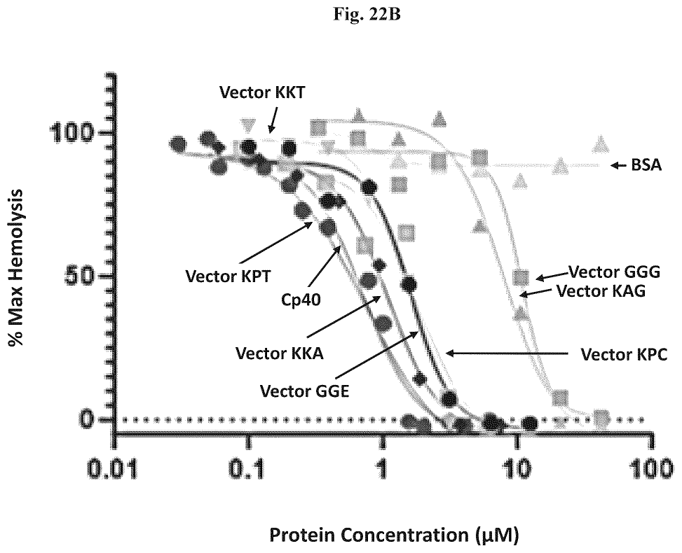

After (DK) C3i-Fc4-CNP36 (Vector GGE) and Fc4-C3i (Vector GGG) were purified, SDS-PAGE analysis showed the purifications were of high purity (FIG. 22A). In the hemolysis inhibition assay, the IC50 for Vector GGE and Vector GGG was 2.37 and 29.05 μM, respectively. Compared with Vector GGG, IC50 value of Vector GGE (CNP36) was decreased by about 12 times. Since CNP36 is not involved in the hemolysis inhibition, it was thought that C3i located at the N-terminus could possess stronger hemolysis inhibition ability. Additionally, it was observed that a fusion protein with C3i located at the C-terminus had poor secretion, but this was not an issue for that with C3i at the N-terminus (Data not shown). Therefore, it was anticipated that switching C3i from C-terminus to N-terminus could not only ameliorate the protein secretion issue, but also efficiently improve fusion protein hemolysis inhibition activities.

The mutant protein, Vector KAG, derived from the Vector GGG, had the C3i(N15Q) at the C-terminus following Fc4 fragment (Fc4-C3i (N15Q)). When the C3i(N15Q) location was changed from C-terminus to N-terminus, a new mutant protein (C3i(N15Q)-Fc4) in Vector KPC was generated. Both proteins were purified and analyzed on a SDS-PAGE gel (FIG. 22A) and confirmed to have high purity. The proteins were then used in a hemolysis inhibition assay. As expected, IC50 value of Vector KPC protein was about 8 times lower than Vector KAG protein (FIG. 22B, Table 21), suggesting Vector KPC and Vector KAG proteins could have different protein structure.

### C3i Installment on Both N-Terminus and C-Terminus

Since C3i located in either N-terminus or C-terminus showed hemolysis inhibition activities, to improve the avidity, C3i(N15Q) was introduced into both N- and C-terminus. A new variant was generated, C3i(N15Q)-Fc4-C3i(N15Q) (Vector KPT). Vector KPT protein was purified and analyzed on a SDS-PAGE gel (FIG. 22A) and confirmed to have high purity. Lane content: M. Protein marker; 1, Vector GGE; 2, Vector GGG; 3, Vector KAG; 4, Vector KKT; 5, Vector KKA; 6, Vector KPT; 7, Vector KPC.

The proteins were then used in a hemolysis inhibition assay with other C3i fusion proteins as well as the benchmark CP40. As shown in the FIG. 22B, Vector KPT protein showed the strongest hemolysis inhibition activities among all C3i fusion proteins and the benchmark CP40. When the data was plotted, IC50 of Vector KPT was estimated to be 0.58 μM, which was even lower than the CP40 (Table 21).

Table 45 summarizes the process of C3i fusion protein engineering with the estimated IC50 comparison. Derived from Vector GGG with C3i at the C-terminus, Vector KAG has a single amino acid mutation in the last position of C3i, Vector KPC has the C3i at the N-terminus, and Vector KPT has C3i at both N- and C-terminus. Despite of the subtle change, these proteins have dramatically different hemolysis inhibition activities. Compared with Vector GGG, IC50 for Vector KAG, Vector KPC, and Vector KPT was decreased by 2.61, 20.46, 50.09 times, respectively. More importantly, the Vector KPT showed a lower IC50 than the benchmark, CP40. Taken together, this data indicated these C3i fusion proteins showed an unexpected and improved technical effect.

TABLE 45
Estimated IC50 for C3i-fusion proteins

| | IC50 (μM) | Ratio (decrease) |
|---|---|---|
| Vector GGG (Fc-C3i) N15Q | 29.05 | 1.00 |
| Vector KAG (Fc-C3i (N15Q)) | 11.14 | 2.61 |
| C’---> N’ | | |
| Vector KPC (C3i (N15Q)) | 1.42 | 20.46 |
| Vector KPT (C3i(N15Q)-Fc- | 0.58 | 50.09 |
| C3i(N15Q)) | | |

Introduction of CNP36 and Endostatin into the Fusion Proteins

Dry AMD is a chronic disease and its pathology remains unclear. An angiogenesis inhibitor, CNP36 and endostatin was fused with C3i(N15Q)-Fc4, generating a C3i fusion protein, C3i(N15Q)-Fc4-CNP36 (Vector KKT) and C3i(N15Q)-Fc4-endostatin (Vector KKA), respectively.

C3i(N15Q)-Fc4-CNP36 (Vector KKT) and C3i(N15Q)-Fc4-endostatin (Vector KKA) proteins were purified by Protein A resins. The purified proteins were run on an SDS-PAGE gel and their purity was assessed (FIG. 22A). The purified Vector KKT and Vector KKA proteins were used in a hemolysis inhibition assay. The results showed that Vector KKT and Vector KKA had the similar IC50 to Vector KPC (Table 21), which confirms that CNP36 and endostatin are not related with the complement pathways.

TABLE 21
Estimated IC50 for C3i-fusion proteins

| Proteins | Description | Estimated IC50 (μM) |
|---|---|---|
| Vector KPT | C3i(N15Q)-Fc- | 0.58 |
| | C3i(N15Q) | |
| CP40 | CP40 peptide | 0.78 |
| Vector KKT | C3i(N15Q)-Fc-CNP36 | 1.29 |
| Vector KPC | C3i(N15Q)-Fc | 1.42 |
| Vector KKA | C3i(N15Q)-Fc- | 1.6 |
| | Endostatin | |
| Vector GGE | C3i-Fc-CNP36 | 2.37 |
| Vector KAG | Fc-C3i(N15Q) | 11.14 |
| Vector GGG | Fc-C3i | 29.05 |

### The Binding Affinity Measurement of C3i Fusion Proteins by Biacore

The estimated IC50 of the C3i fusion proteins were measured as shown above. Next their binding affinity with Complement C3b, C3c, and C3 was tested. Binding affinity was measured using Biacore. Biosensor analysis was performed at room temperature in a running buffer (1×HBS, 20 mM NaH₂PO₄-Na₂HPO₄·H₂O, 150 mM NaCl, pH 7.4, supplemented with 0.005% (v/v) surfactant P20), using a Biacore 3000 optical biosensor equipped with a research-grade CM5 sensor chip. C3, C3b and C3c proteins were immobilized in flow cell 2 or 4 via standard amine coupling, leaving Fc1 or 3 unmodified to serve as a reference surface. The immobilization levels of proteins were calculated using Rmax=100 RU and the ratio of ligand molecular weight/analyte molecular. Data analysis was conducted using the BIAevaluation software. All sensorgrams were double-referenced by subtracting the response on a reference flow cell and a blank sample. Five 2× serial dilution samples were injected sequentially from low concentration to high concentration of proteins at a flow rate of 30 μL/min (300 seconds for each), followed by a dissociation time of 900 seconds. Calculations of kinetic parameters were conducted assuming a simple 1:1 (Langmuir) binding.

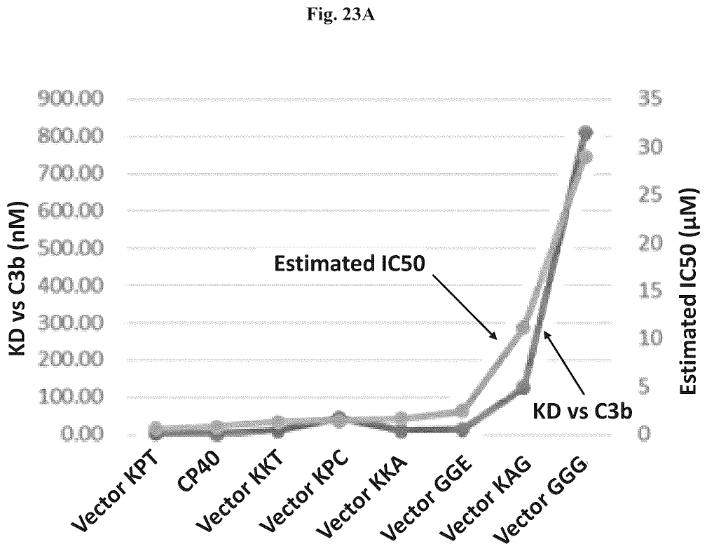

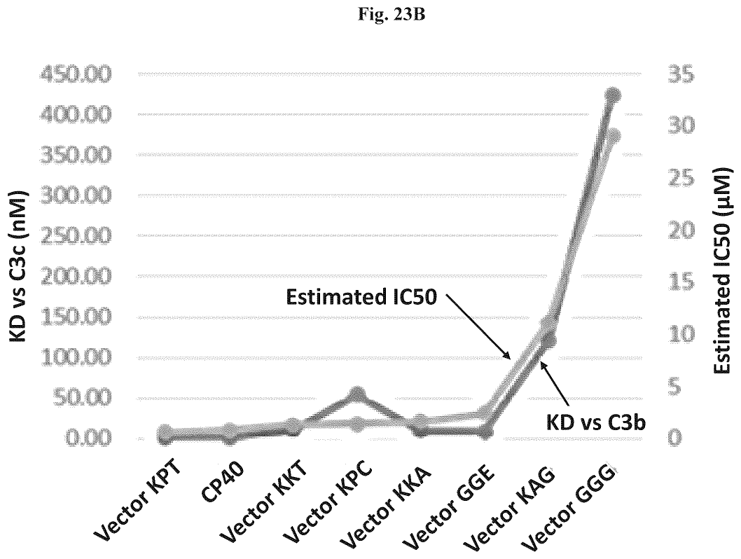

After the testing concentrating was optimized for individual proteins, they were applied on the immobilized chip, and association rate constant (Ka, M⁻¹s⁻¹), dissociation constant (Kd, s⁻¹), equilibrium dissociation constant (KD, M), maximal feasible signal (Rₘₐₓ), and chi² were recorded. The results for complement C3b, C3c, and C3 are listed in Table 22, Table 23, and Table 24, respectively. The results of the experiments showed that CP40 binding affinity against C3b and C3c was similar to previously published results, indicating the testing conditions were well optimized. The estimated IC50 of C3i and its fusion proteins and their binding affinity (KD) against C3b, C3c, and C3 are shown in Table 25. When KD values were plotted against estimated IC50, they correlated well with each other for C3b (FIG. 23A) and C3c (FIG. 23B) and, in most cases, for C3 (FIG. 23C). The Vector GGG was shown to have relatively high binding affinity with C3, which was inconsistent with the IC50 value.

TABLE 22
Binding affinity of C3i fusion proteins
and CP40 against C3b

| Proteins | ka (M⁻¹S⁻¹) | kd (S⁻¹) | KD (M) | Rmax | Chi² |
|---|---|---|---|---|---|
| CP40 | 2.67E+06 | 8.66E−04 | 3.24E−10 | 3.18 | 0.12 |
| Vector KPT | 1.90E+05 | 1.83E−04 | 9.60E−10 | 98.60 | 0.34 |
| Vector KKT | 3.69E+09 | 3.56E+01 | 9.63E−09 | 51.8 | 4.81 |
| Vector KKA | 7.46E+06 | 6.87E−02 | 9.20E−09 | 74.5 | 9.81 |
| Vector KPC | 2.62E+05 | 1.12E−02 | 4.29E−08 | 12.5 | 0.948 |
| Vector GGE | 4.98E+05 | 6.34E−03 | 1.27E−08 | 45.60 | 3.97 |
| Vector KAG | 6.30E+04 | 7.94E−03 | 1.25E−07 | 18.4 | 0.74 |
| Vector GGG | 9.21E+03 | 7.46E−03 | 8.10E−07 | 30.7 | 0.408 |

TABLE 23
Binding affinity of C3i fusion proteins and CP40 against C3c

| Proteins | ka (M⁻¹S⁻¹) | kd (S⁻¹) | KD (M) | Rmax | Chi² |
|---|---|---|---|---|---|
| CP40 | 8.32E+05 | 4.24E−04 | 5.09E−10 | 4.65 | 0.05 |
| Vector KPT | 3.76E+05 | 1.44E−04 | 3.82E−10 | 147.00 | 0.26 |
| Vector KKT | 4.98E+06 | 5.36E−02 | 1.08E−08 | 29.70 | 0.67 |
| Vector KKA | 3.12E+06 | 2.69E−02 | 8.62E−09 | 53.50 | 4.39 |
| Vector KPC | 5.43E+04 | 2.91E−03 | 5.35E−08 | 18.50 | 0.25 |
| Vector GGE | 1.27E+06 | 1.01E−02 | 7.97E−09 | 42.20 | 2.12 |
| Vector KAG | 7.50E+04 | 9.05E−03 | 1.21E−07 | 14.50 | 0.20 |
| Vector GGG | 9.77E+03 | 4.14E−03 | 4.24E−07 | 25.00 | 0.25 |

TABLE 24
Binding affinity of C3i fusion proteins and CP40 against C3

| Proteins | ka (M⁻¹S⁻¹) | kd (S⁻¹) | KD (M) | Rmax | Chi² |
|---|---|---|---|---|---|
| CP40 | 1.37E+06 | 8.62E−04 | 6.29E−10 | 25.70 | 0.10 |
| Vector KPT | 1.35E+05 | 7.64E−04 | 5.67E−09 | 28.70 | 0.12 |
| Vector KKT | 7.74E+05 | 3.64E−03 | 4.71E−09 | 15.80 | 0.75 |
| Vector KKA | 1.32E+06 | 3.07E−03 | 2.31E−09 | 10.10 | 0.79 |
| Vector KPC | 8.64E+02 | 1.18E−06 | 1.37E−09 | 136.00 | 0.37 |
| Vector GGE | 1.00E+05 | 4.91E−03 | 4.89E−08 | 28.80 | 0.37 |
| Vector KAG | 6.17E+04 | 3.10E−03 | 5.03E−08 | 7.17 | 0.48 |
| Vector GGG | 9.75E+04 | 2.47E−03 | 2.54E−08 | 3.23 | 0.30 |

TABLE 25
The correlation between binding affinity and estimated IC50

| | Bioactivity | | Affinity to C3 | | Affinity to C3b | | Affinity to C3c | |
|---|---|---|---|---|---|---|---|---|
| | IC50 | Change | KD | Change | KD | Change | KD | Change |
| Proteins | (μM) | (fold) | (nM) | (fold) | (nM) | (fold) | (nM) | (fold) |
| CP40 | 0.78 | 1 | 0.63 | 1 | 0.32 | 1 | 0.51 | 1 |
| Vector | 0.58 | 1.3 | 5.67 | −9 | 0.96 | −3 | 0.38 | 1.3 |
| KPT/Vector | | | | | | | | |
| KMR | | | | | | | | |
| Vector | 1.29 | −1.7 | 4.71 | −7.5 | 9.63 | −30 | 10.8 | −21 |
| KKT | | | | | | | | |
| Vector | 1.6 | −2.1 | 2.31 | −3.7 | 9.2 | −29 | 8.62 | −17 |
| KKA | | | | | | | | |
| Vector | 1.42 | −1.8 | 1.37 | −2.2 | 42.9 | −134 | 53.5 | −105 |
| KPC | | | | | | | | |
| Vector | 2.37 | −3.03 | 48.9 | −78 | 12.7 | −37.5 | 7.97 | −15.6 |
| GGE | | | | | | | | |
| Vector | 11.14 | −14.62 | 50.3 | −80 | 125 | −390 | 121 | −237.3 |
| KAG | | | | | | | | |
| Vector | 29.05 | −37.24 | 25.4 | −40 | 810 | −2531 | 424 | −831.3 |
| GGG | | | | | | | | |

TABLE 26
DNA sequences of plasmids used in Example 8

| Vector | | | |
|---|---|---|---|
| name | Protein | Nucleic acid sequence (ORF for GOI) | SEQ ID NO |
| Vector | Fc-C3i, | ATGGAGTTCGGCCTGAGCTGGCTGTTCCTGGTGGCCATC | 259 |
| KTP | T14A | CTTAAGGGCGTGCAGTGCGACAAGAGAGTGGAGAGCAA | |
| | | GTACGGCCCCCCCTGCCCCCCCTGCCCCGCCCCCGAGTT | |
| | | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCATCTGCGTGTGGCAGGACTGGGGC | |
| | | GCCCACCGCTGCGCCAACTAA | |
| Vector | Fc-C3i, | ATGGAGTTCGGCCTGAGCTGGCTGTTCCTGGTGGCCATC | 260 |
| KTQ | +2Y | CTTAAGGGCGTGCAGTGCGACAAGAGAGTGGAGAGCAA | |
| | | GTACGGCCCCCCCTGCCCCCCCTGCCCCGCCCCCGAGTT | |
| | | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCTACATCTGCGTGTGGCAGGACTGGG | |
| | | GCGCCCACCGCTGCACCAACTAA | |
| Vector | Fc-C3i, | ATGGAGTTCGGCCTGAGCTGGCTGTTCCTGGTGGCCATC | 261 |
| KTR | +2Y, | CTTAAGGGCGTGCAGTGCGACAAGAGAGTGGAGAGCAA | |
| | T14A | GTACGGCCCCCCCTGCCCCCCCTGCCCCGCCCCCGAGTT | |
| | | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCTACATCTGCGTGTGGCAGGACTGGG | |
| | | GCGCCCACCGCTGCGCCAACTAA | |
| Vector | Fc-C3i,- | ATGGAGTTCGGCCTGAGCTGGCTGTTCCTGGTGGCCATC | 262 |
| KAT | N15 | CTTAAGGGCGTGCAGTGCGACAAGAGAGTGGAGAGCAA | |
| | | GTACGGCCCCCCCTGCCCCCCCTGCCCCGCCCCCGAGTT | |
| | | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCATCTGCGTGTGGCAGGACTGGGGC | |
| | | GCCCACCGCTGCACCTAA | |
| Vector | Fc-C3i, | ATGGAGTTCGGCCTGAGCTGGCTGTTCCTGGTGGCCATC | 263 |
| KAA | T14A,- | CTTAAGGGCGTGCAGTGCGACAAGAGAGTGGAGAGCAA | |
| | N15 | GTACGGCCCCCCCTGCCCCCCCTGCCCCGCCCCCGAGTT | |
| | | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCATCTGCGTGTGGCAGGACTGGGGC | |
| | | GCCCACCGCTGCGCCTAA | |
| Vector | Fc-C3i, | ATGGAGTTCGGCCTGAGCTGGCTGTTCCTGGTGGCCATC | 264 |
| KAC | +2Y,- | CTTAAGGGCGTGCAGTGCGACAAGAGAGTGGAGAGCAA | |
| | N15 | GTACGGCCCCCCCTGCCCCCCCTGCCCCGCCCCCGAGTT | |
| | | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCTACATCTGCGTGTGGCAGGACTGGG | |
| | | GCGCCCACCGCTGCACCTAA | |
| Vector | Fc-C3i, | ATGGAGTTCGGCCTGAGCTGGCTGTTCCTGGTGGCCATC | 265 |
| KAE | +2Y, | CTTAAGGGCGTGCAGTGCGACAAGAGAGTGGAGAGCAA | |
| | T14A,- | GTACGGCCCCCCCTGCCCCCCCTGCCCCGCCCCCGAGTT | |
| | N15 | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCTACATCTGCGTGTGGCAGGACTGGG | |
| | | GCGCCCACCGCTGCGCCTAA | |
| Vector | Fc-C3i, | ATGGAGTTCGGCCTGAGCTGGCTGTTCCTGGTGGCCATC | 266 |
| KAG | N15Q | CTTAAGGGCGTGCAGTGCGACAAGAGAGTGGAGAGCAA | |
| | | GTACGGCCCCCCCTGCCCCCCCTGCCCCGCCCCCGAGTT | |
| | | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCATCTGCGTGTGGCAGGACTGGGGC | |
| | | GCCCACCGCTGCACCCAGTAA | |
| Vector | Fc-C3i, | ATGGAGTTCGGCCTGAGCTGGCTGTTCCTGGTGGCCATC | 267 |
| KAK | T14A, | CTTAAGGGCGTGCAGTGCGACAAGAGAGTGGAGAGCAA | |
| | N15Q | GTACGGCCCCCCCTGCCCCCCCTGCCCCGCCCCCGAGTT | |
| | | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCATCTGCGTGTGGCAGGACTGGGGC | |
| | | GCCCACCGCTGCGCCCAGTAA | |
| Vector | Fc-C3i, | ATGGAGTTCGGCCTGAGCTGGCTGTTCCTGGTGGCCATC | 268 |
| KAM | +2Y, | CTTAAGGGCGTGCAGTGCGACAAGAGAGTGGAGAGCAA | |
| | N15Q | GTACGGCCCCCCCTGCCCCCCCTGCCCCGCCCCCGAGTT | |
| | | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCTACATCTGCGTGTGGCAGGACTGGG | |
| | | GCGCCCACCGCTGCACCCAGTAA | |
| Vector | Fc-C3i, | ATGGAGTTCGGCCTGAGCTGGCTGTTCCTGGTGGCCATC | 269 |
| KAP | +2Y, | CTTAAGGGCGTGCAGTGCGACAAGAGAGTGGAGAGCAA | |
| | T14A, | GTACGGCCCCCCCTGCCCCCCCTGCCCCGCCCCCGAGTT | |
| | N15Q | CCTGGGCGGCCCCAGCGTGTTCCTGTTCCCCCCCAAGCC | |
| | | CAAGGACACCCTGATGATCAGCAGAACCCCCGAGGTGA | |
| | | CCTGCGTGGTGGTGGACGTGAGCCAGGAGGACCCCGAG | |
| | | GTGCAGTTCAACTGGTACGTGGACGGCGTGGAGGTGCAC | |
| | | AACGCCAAGACCAAGCCCAGAGAGGAGCAGTTCAACAG | |
| | | CACCTACAGAGTGGTGAGCGTGCTGACCGTGCTGCACCA | |
| | | GGACTGGCTGAACGGCAAGGAGTACAAGTGCAAGGTGA | |
| | | GCAACAAGGGCCTGCCCAGCAGCATCGAGAAGACCATC | |
| | | AGCAAGGCCAAGGGCCAGCCCAGAGAGCCCCAGGTGTA | |
| | | CACCCTGCCCCCCAGCCAGGAGGAGATGACCAAGAACC | |
| | | AGGTGAGCCTGACCTGCCTGGTGAAGGGCTTCTACCCCA | |
| | | GCGACATCGCCGTGGAGTGGGAGAGCAACGGCCAGCCC | |
| | | GAGAACAACTACAAGACCACCCCCCCCGTGCTGGACAG | |
| | | CGACGGCAGCTTCTTCCTGTACAGCAGACTGACCGTGGA | |
| | | CAAGAGCAGATGGCAGGAGGGCAACGTGTTCAGCTGCA | |
| | | GCGTGATGCACGAGGCCCTGCACAACCACTACACCCAG | |
| | | AAGAGCCTGAGCCTGAGCCTGGGCAAGGGCGGAGGCGG | |
| | | AAGCGGCGGAGGCGGATCTGGCGGAGGCGGAAGCGGCG | |
| | | GAGGCGGCTCTGGCTACATCTGCGTGTGGCAGGACTGGG | |
| | | GCGCCCACCGCTGCGCCCAGTAA | |
| Vector | C3i(N15 | ATGGAGTTCGGCCTGAGCTGGCTGTTCCTGGTGGCCATC | 272 |
| KKT | Q)-Fc4- | CTTAAGGGCGTGCAGTGCGACAAGGGCATCTGCGTGTGG | |
| | CNP36 | CAGGACTGGGGCGCCCACCGCTGCACCCAGGGCGGAGG | |
| | | CGGAAGCGGCGGAGGCGGATCTGGCGGAGGCGGAAGCG | |
| | | ACAAGAGAGTGGAGAGCAAGTACGGCCCCCCCTGCCCC | |
| | | CCCTGCCCCGCCCCCGAGTTCCTGGGCGGCCCCAGCGTG | |
| | | TTCCTGTTCCCCCCCAAGCCCAAGGACACCCTGATGATC | |
| | | AGCAGAACCCCCGAGGTGACCTGCGTGGTGGTGGACGT | |
| | | GAGCCAGGAGGACCCCGAGGTGCAGTTCAACTGGTACG | |
| | | TGGACGGCGTGGAGGTGCACAACGCCAAGACCAAGCCC | |
| | | AGAGAGGAGCAGTTCAACAGCACCTACAGAGTGGTGAG | |
| | | CGTGCTGACCGTGCTGCACCAGGACTGGCTGAACGGCAA | |
| | | GGAGTACAAGTGCAAGGTGAGCAACAAGGGCCTGCCCA | |
| | | GCAGCATCGAGAAGACCATCAGCAAGGCCAAGGGCCAG | |
| | | CCCAGAGAGCCCCAGGTGTACACCCTGCCCCCCAGCCAG | |
| | | GAGGAGATGACCAAGAACCAGGTGAGCCTGACCTGCCT | |
| | | GGTGAAGGGCTTCTACCCCAGCGACATCGCCGTGGAGTG | |
| | | GGAGAGCAACGGCCAGCCCGAGAACAACTACAAGACCA | |
| | | CCCCCCCCGTGCTGGACAGCGACGGCAGCTTCTTCCTGT | |
| | | ACAGCAGACTGACCGTGGACAAGAGCAGATGGCAGGAG | |
| | | GGCAACGTGTTCAGCTGCAGCGTGATGCACGAGGCCCTG | |
| | | CACAACCACTACACCCAGAAGAGCCTGAGCCTGAGCCT | |
| | | GGGCAAGGGCGGAGGCGGAAGCGGCGGAGGCGGATCTG | |
| | | GCGGAGGCGGAAGCGGCGGAGGCGGCTCTGAGCACCCC | |
| | | AACGCGCGCAAATACAAAGGAGCCAACAAGAAGGGCTT | |
| | | GTCCAAGGGCTGCTTCGGCCTCAAGCTGGACCGAATCGG | |
| | | CTCCATGAGCGGCCTGGGATGTTAG | |
| Vector | C3i(N15 | ATGGAGTTCGGCCTGAGCTGGCTGTTCCTGGTGGCCATC | 273 |
| KKA | Q)-Fc4- | CTTAAGGGCGTGCAGTGCGACAAGGGCATCTGCGTGTGG | |
| | Endostatin | CAGGACTGGGGCGCCCACCGCTGCACCCAGGGCGGAGG | |
| | | CGGAAGCGGCGGAGGCGGATCTGGCGGAGGCGGAAGCG | |
| | | ACAAGAGAGTGGAGAGCAAGTACGGCCCCCCCTGCCCC | |
| | | CCCTGCCCCGCCCCCGAGTTCCTGGGCGGCCCCAGCGTG | |
| | | TTCCTGTTCCCCCCCAAGCCCAAGGACACCCTGATGATC | |
| | | AGCAGAACCCCCGAGGTGACCTGCGTGGTGGTGGACGT | |
| | | GAGCCAGGAGGACCCCGAGGTGCAGTTCAACTGGTACG | |
| | | TGGACGGCGTGGAGGTGCACAACGCCAAGACCAAGCCC | |
| | | AGAGAGGAGCAGTTCAACAGCACCTACAGAGTGGTGAG | |
| | | CGTGCTGACCGTGCTGCACCAGGACTGGCTGAACGGCAA | |
| | | GGAGTACAAGTGCAAGGTGAGCAACAAGGGCCTGCCCA | |
| | | GCAGCATCGAGAAGACCATCAGCAAGGCCAAGGGCCAG | |
| | | CCCAGAGAGCCCCAGGTGTACACCCTGCCCCCCAGCCAG | |
| | | GAGGAGATGACCAAGAACCAGGTGAGCCTGACCTGCCT | |
| | | GGTGAAGGGCTTCTACCCCAGCGACATCGCCGTGGAGTG | |
| | | GGAGAGCAACGGCCAGCCCGAGAACAACTACAAGACCA | |
| | | CCCCCCCCGTGCTGGACAGCGACGGCAGCTTCTTCCTGT | |
| | | ACAGCAGACTGACCGTGGACAAGAGCAGATGGCAGGAG | |
| | | GGCAACGTGTTCAGCTGCAGCGTGATGCACGAGGCCCTG | |
| | | CACAACCACTACACCCAGAAGAGCCTGAGCCTGAGCCT | |
| | | GGGCAAGGGCGGAGGCGGAAGCGGCGGAGGCGGATCTG | |
| | | GCGGAGGCGGAAGCGGCGGAGGCGGCTCTCACAGCCAC | |
| | | AGGGACTTCCAGCCTGTGCTGCACCTGGTGGCCCTGAAC | |
| | | AGCCCTCTGAGCGGAGGAATGAGGGGAATCAGGGGAGC | |
| | | CGACTTCCAGTGCTTCCAGCAGGCCAGGGCCGTGGGACT | |
| | | GGCCGGAACCTTCAGGGCCTTCCTGAGCAGCAGGCTGCA | |
| | | GGACCTGTACAGCATCGTGAGGAGGGCCGACAGGGCCG | |
| | | CCGTGCCCATCGTGAACCTGAAGGACGAGCTGCTGTTCC | |
| | | CTAGCTGGGAGGCCCTGTTCAGCGGAAGCGAGGGACCC | |
| | | CTGAAGCCCGGAGCCAGGATCTTCAGCTTCGACGGAAA | |
| | | GGACGTGCTGAGGCACCCTACCTGGCCTCAGAAGAGCGT | |
| | | GTGGCACGGAAGCGACCCTAACGGAAGGAGGCTGACCG | |
| | | AGAGCTACTGCGAGACCTGGAGGACCGAGGCCCCTAGC | |
| | | GCCACCGGCCAGGCCAGCAGCCTGCTGGGAGGAAGGCT | |
| | | GCTGGGACAGAGCGCCGCCAGCTGCCACCACGCCTACA | |
| | | TCGTGCTGTGCATCGAGAACAGCTTCATGACCGCCAGCA | |
| | | AGTGA | |
| Vector | C3i(N15 | ATGGAGTTCGGCCTGAGCTGGCTGTTCCTGGTGGCCATC | 274 |
| | | CTTAAGGGCGTGCAGTGCGACAAGGGCATCTGCGTGTGG | |
| | | CAGGACTGGGGCGCCCACCGCTGCACCCAGGGCGGAGG | |
| | | CGGAAGCGGCGGAGGCGGATCTGGCGGAGGCGGAAGCG | |
| | | ACAAGAGAGTGGAGAGCAAGTACGGCCCCCCCTGCCCC | |
| | | CCCTGCCCCGCCCCCGAGTTCCTGGGCGGCCCCAGCGTG | |
| | | TTCCTGTTCCCCCCCAAGCCCAAGGACACCCTGATGATC | |
| KMR, | Q)-Fc4- | AGCAGAACCCCCGAGGTGACCTGCGTGGTGGTGGACGT | |
| Vector | C3i(N15Q) | GAGCCAGGAGGACCCCGAGGTGCAGTTCAACTGGTACG | |
| KPT | | TGGACGGCGTGGAGGTGCACAACGCCAAGACCAAGCCC | |
| | | AGAGAGGAGCAGTTCAACAGCACCTACAGAGTGGTGAG | |
| | | CGTGCTGACCGTGCTGCACCAGGACTGGCTGAACGGCAA | |
| | | GGAGTACAAGTGCAAGGTGAGCAACAAGGGCCTGCCCA | |
| | | GCAGCATCGAGAAGACCATCAGCAAGGCCAAGGGCCAG | |
| | | CCCAGAGAGCCCCAGGTGTACACCCTGCCCCCCAGCCAG | |
| | | GAGGAGATGACCAAGAACCAGGTGAGCCTGACCTGCCT | |
| | | GGTGAAGGGCTTCTACCCCAGCGACATCGCCGTGGAGTG | |
| | | GGAGAGCAACGGCCAGCCCGAGAACAACTACAAGACCA | |
| | | CCCCCCCCGTGCTGGACAGCGACGGCAGCTTCTTCCTGT | |
| | | ACAGCAGACTGACCGTGGACAAGAGCAGATGGCAGGAG | |
| | | GGCAACGTGTTCAGCTGCAGCGTGATGCACGAGGCCCTG | |
| | | CACAACCACTACACCCAGAAGAGCCTGAGCCTGAGCCT | |
| | | GGGCAAGGGCGGAGGCGGAAGCGGCGGAGGCGGATCTG | |
| | | GCGGAGGCGGAAGCGGCGGAGGCGGCTCTGGCATCTGC | |
| | | GTGTGGCAGGACTGGGGCGCCCACCGCTGCACCCAGTA | |
| | | A | |
| Vector | C3i(N15 | ATGGAGTTCGGCCTGAGCTGGCTGTTCCTGGTGGCCATC | 275 |
| KPC | Q)-Fc4 | CTTAAGGGCGTGCAGTGCGACAAGGGCATCTGCGTGTGG | |
| | | CAGGACTGGGGCGCCCACCGCTGCACCCAGGGCGGAGG | |
| | | CGGAAGCGGCGGAGGCGGATCTGGCGGAGGCGGAAGCG | |
| | | ACAAGAGAGTGGAGAGCAAGTACGGCCCCCCCTGCCCC | |
| | | CCCTGCCCCGCCCCCGAGTTCCTGGGCGGCCCCAGCGTG | |
| | | TTCCTGTTCCCCCCCAAGCCCAAGGACACCCTGATGATC | |
| | | AGCAGAACCCCCGAGGTGACCTGCGTGGTGGTGGACGT | |
| | | GAGCCAGGAGGACCCCGAGGTGCAGTTCAACTGGTACG | |
| | | TGGACGGCGTGGAGGTGCACAACGCCAAGACCAAGCCC | |
| | | AGAGAGGAGCAGTTCAACAGCACCTACAGAGTGGTGAG | |
| | | CGTGCTGACCGTGCTGCACCAGGACTGGCTGAACGGCAA | |
| | | GGAGTACAAGTGCAAGGTGAGCAACAAGGGCCTGCCCA | |
| | | GCAGCATCGAGAAGACCATCAGCAAGGCCAAGGGCCAG | |
| | | CCCAGAGAGCCCCAGGTGTACACCCTGCCCCCCAGCCAG | |
| | | GAGGAGATGACCAAGAACCAGGTGAGCCTGACCTGCCT | |
| | | GGTGAAGGGCTTCTACCCCAGCGACATCGCCGTGGAGTG | |
| | | GGAGAGCAACGGCCAGCCCGAGAACAACTACAAGACCA | |
| | | CCCCCCCCGTGCTGGACAGCGACGGCAGCTTCTTCCTGT | |
| | | ACAGCAGACTGACCGTGGACAAGAGCAGATGGCAGGAG | |
| | | GGCAACGTGTTCAGCTGCAGCGTGATGCACGAGGCCCTG | |
| | | CACAACCACTACACCCAGAAGAGCCTGAGCCTGAGCCT | |
| | | GGGCAAGTAA | |

TABLE 27
Peptides and fusion protein sequences of Example 8

| Vector | | | |
|---|---|---|---|
| name | Protein | Nucleic acid sequence (ORF for GOI) | SEQ ID NO |
| Vector | Fc-C3i, | MEFGLSWLFLVAILKGVQCDKRVESKYGPPCPPCPAPEFL | 286 |
| KTP | T14A | GGPSVFLFPPKPKDTLMISRTPEVTCVVVDVSQEDPEVQFN | |
| | | WYVDGVEVHNAKTKPREEQFNSTYRVVSVLTVLHQDWL | |
| | | NGKEYKCKVSNKGLPSSIEKTISKAKGQPREPQVYTLPPSQ | |
| | | EEMTKNQVSLTCLVKGFYPSDIAVEWESNGQPENNYKTT | |
| | | PPVLDSDGSFFLYSRLTVDKSRWQEGNVFSCSVMHEALH | |
| | | NHYTQKSLSLSLGKGGGGSGGGGSGGGGSGGGGSGICVW | |
| | | QDWGAHRCAN | |
| Vector | Fc-C3i, | MEFGLSWLFLVAILKGVQCDKRVESKYGPPCPPCPAPEFL | 287 |
| KTQ | +2Y | GGPSVFLFPPKPKDTLMISRTPEVTCVVVDVSQEDPEVQFN | |
| | | WYVDGVEVHNAKTKPREEQFNSTYRVVSVLTVLHQDWL | |
| | | NGKEYKCKVSNKGLPSSIEKTISKAKGQPREPQVYTLPPSQ | |
| | | EEMTKNQVSLTCLVKGFYPSDIAVEWESNGQPENNYKTT | |
| | | PPVLDSDGSFFLYSRLTVDKSRWQEGNVFSCSVMHEALH | |
| | | NHYTQKSLSLSLGKGGGGSGGGGSGGGGSGGGGSGYICV | |
| | | WQDWGAHRCTN | |
| Vector | Fc-C3i, | MEFGLSWLFLVAILKGVQCDKRVESKYGPPCPPCPAPEFL | 288 |
| KTR | +2Y, | GGPSVFLFPPKPKDTLMISRTPEVTCVVVDVSQEDPEVQFN | |
| | T14A | WYVDGVEVHNAKTKPREEQFNSTYRVVSVLTVLHQDWL | |
| | | NGKEYKCKVSNKGLPSSIEKTISKAKGQPREPQVYTLPPSQ | |
| | | EEMTKNQVSLTCLVKGFYPSDIAVEWESNGQPENNYKTT | |
| | | PPVLDSDGSFFLYSRLTVDKSRWQEGNVFSCSVMHEALH | |
| | | NHYTQKSLSLSLGKGGGGSGGGGSGGGGSGGGGSGYICV | |
| | | WQDWGAHRCAN | |
| Vector | Fc-C3i,- | MEFGLSWLFLVAILKGVQCDKRVESKYGPPCPPCPAPEFL | 289 |
| KAT | N15 | GGPSVFLFPPKPKDTLMISRTPEVTCVVVDVSQEDPEVQFN | |
| | | WYVDGVEVHNAKTKPREEQFNSTYRVVSVLTVLHQDWL | |
| | | NGKEYKCKVSNKGLPSSIEKTISKAKGQPREPQVYTLPPSQ | |
| | | EEMTKNQVSLTCLVKGFYPSDIAVEWESNGQPENNYKTT | |
| | | PPVLDSDGSFFLYSRLTVDKSRWQEGNVFSCSVMHEALH | |
| | | NHYTQKSLSLSLGKGGGGSGGGGSGGGGSGGGGSGICVW | |
| | | QDWGAHRCT | |
| Vector | Fc-C3i, | MEFGLSWLFLVAILKGVQCDKRVESKYGPPCPPCPAPEFL | 290 |
| KAA | T14A,- | GGPSVFLFPPKPKDTLMISRTPEVTCVVVDVSQEDPEVQFN | |
| | N15 | WYVDGVEVHNAKTKPREEQFNSTYRVVSVLTVLHQDWL | |
| | | NGKEYKCKVSNKGLPSSIEKTISKAKGQPREPQVYTLPPSQ | |
| | | EEMTKNQVSLTCLVKGFYPSDIAVEWESNGQPENNYKTT | |
| | | PPVLDSDGSFFLYSRLTVDKSRWQEGNVFSCSVMHEALH | |
| | | NHYTQKSLSLSLGKGGGGSGGGGSGGGGSGGGGSGICVW | |
| | | QDWGAHRCA | |
| Vector | Fc-C3i, | MEFGLSWLFLVAILKGVQCDKRVESKYGPPCPPCPAPEFL | 291 |
| KAC | +2Y,- | GGPSVFLFPPKPKDTLMISRTPEVTCVVVDVSQEDPEVQFN | |
| | N15 | WYVDGVEVHNAKTKPREEQFNSTYRVVSVLTVLHQDWL | |
| | | NGKEYKCKVSNKGLPSSIEKTISKAKGQPREPQVYTLPPSQ | |
| | | EEMTKNQVSLTCLVKGFYPSDIAVEWESNGQPENNYKIT | |
| | | PPVLDSDGSFFLYSRLTVDKSRWQEGNVFSCSVMHEALH | |
| | | NHYTQKSLSLSLGKGGGGSGGGGSGGGGSGGGGSGYICV | |
| | | WQDWGAHRCT | |
| Vector | Fc-C3i, | MEFGLSWLFLVAILKGVQCDKRVESKYGPPCPPCPAPEFL | 292 |
| KAE | +2Y, | GGPSVFLFPPKPKDTLMISRTPEVTCVVVDVSQEDPEVQFN | |
| | T14A,- | WYVDGVEVHNAKTKPREEQFNSTYRVVSVLTVLHQDWL | |
| | N15 | NGKEYKCKVSNKGLPSSIEKTISKAKGQPREPQVYTLPPSQ | |
| | | EEMTKNQVSLTCLVKGFYPSDIAVEWESNGQPENNYKTT | |
| | | PPVLDSDGSFFLYSRLTVDKSRWQEGNVFSCSVMHEALH | |
| | | NHYTQKSLSLSLGKGGGGSGGGGSGGGGSGGGGSGYICV | |
| | | WQDWGAHRCA | |
| Vector | Fc-C3i, | MEFGLSWLFLVAILKGVQCDKRVESKYGPPCPPCPAPEFL | 293 |
| KAG | N15Q | GGPSVFLFPPKPKDTLMISRTPEVTCVVVDVSQEDPEVQFN | |
| | | WYVDGVEVHNAKTKPREEQFNSTYRVVSVLTVLHQDWL | |
| | | NGKEYKCKVSNKGLPSSIEKTISKAKGQPREPQVYTLPPSQ | |
| | | EEMTKNQVSLTCLVKGFYPSDIAVEWESNGQPENNYKTT | |
| | | PPVLDSDGSFFLYSRLTVDKSRWQEGNVFSCSVMHEALH | |
| | | NHYTQKSLSLSLGKGGGGSGGGGSGGGGSGGGGSGICVW | |
| | | QDWGAHRCTQ | |
| Vector | Fc-C3i, | MEFGLSWLFLVAILKGVQCDKRVESKYGPPCPPCPAPEFL | 294 |
| KAK | T14A, | GGPSVFLFPPKPKDTLMISRTPEVTCVVVDVSQEDPEVQFN | |
| | N15Q | WYVDGVEVHNAKTKPREEQFNSTYRVVSVLTVLHQDWL | |
| | | NGKEYKCKVSNKGLPSSIEKTISKAKGQPREPQVYTLPPSQ | |
| | | EEMTKNQVSLTCLVKGFYPSDIAVEWESNGQPENNYKTT | |
| | | PPVLDSDGSFFLYSRLTVDKSRWQEGNVFSCSVMHEALH | |
| | | NHYTQKSLSLSLGKGGGGSGGGGSGGGGSGGGGSGICVW | |
| | | QDWGAHRCAQ | |
| Vector | Fc-C3i, | MEFGLSWLFLVAILKGVQCDKRVESKYGPPCPPCPAPEFL | 295 |
| KAM | +2Y, | GGPSVFLFPPKPKDTLMISRTPEVTCVVVDVSQEDPEVQFN | |
| | N15Q | WYVDGVEVHNAKTKPREEQFNSTYRVVSVLTVLHQDWL | |
| | | NGKEYKCKVSNKGLPSSIEKTISKAKGQPREPQVYTLPPSQ | |
| | | EEMTKNQVSLTCLVKGFYPSDIAVEWESNGQPENNYKTT | |
| | | PPVLDSDGSFFLYSRLTVDKSRWQEGNVFSCSVMHEALH | |
| | | NHYTQKSLSLSLGKGGGGSGGGGSGGGGSGGGGSGYICV | |
| | | WQDWGAHRCTQ | |
| Vector | Fc-C3i, | MEFGLSWLFLVAILKGVQCDKRVESKYGPPCPPCPAPEFL | 296 |
| KAP | +2Y, | GGPSVFLFPPKPKDTLMISRTPEVTCVVVDVSQEDPEVQFN | |
| | T14A, | WYVDGVEVHNAKTKPREEQFNSTYRVVSVLTVLHQDWL | |
| | N15Q | NGKEYKCKVSNKGLPSSIEKTISKAKGQPREPQVYTLPPSQ | |
| | | EEMTKNQVSLTCLVKGFYPSDIAVEWESNGQPENNYKTT | |
| | | PPVLDSDGSFFLYSRLTVDKSRWQEGNVFSCSVMHEALH | |
| | | NHYTQKSLSLSLGKGGGGSGGGGSGGGGSGGGGSGYICV | |
| | | WQDWGAHRCAQ | |
| Vector | C3i(N15Q)- | MEFGLSWLFLVAILKGVQCDKGICVWQDWGAHRCTQGG | 299 |
| KKT | Fc4- | GGSGGGGSGGGGSDKRVESKYGPPCPPCPAPEFLGGPSVF | |
| | CNP36 | LFPPKPKDTLMISRTPEVTCVVVDVSQEDPEVQFNWYVDG | |
| | | VEVHNAKTKPREEQFNSTYRVVSVLTVLHQDWLNGKEY | |
| | | KCKVSNKGLPSSIEKTISKAKGQPREPQVYTLPPSQEEMTK | |
| | | NQVSLTCLVKGFYPSDIAVEWESNGQPENNYKTTPPVLDS | |
| | | DGSFFLYSRLTVDKSRWQEGNVFSCSVMHEALHNHYTQK | |
| | | SLSLSLGKGGGGSGGGGSGGGGSGGGGSEHPNARKYKGA | |
| | | NKKGLSKGCFGLKLDRIGSMSGLGC | |
| Vector | C3i(N15Q)- | MEFGLSWLFLVAILKGVQCDKGICVWQDWGAHRCTQGG | 300 |
| KKA | Fc4- | GGSGGGGSGGGGSDKRVESKYGPPCPPCPAPEFLGGPSVF | |
| | Endostatin | LFPPKPKDTLMISRTPEVTCVVVDVSQEDPEVQFNWYVDG | |
| | | VEVHNAKTKPREEQFNSTYRVVSVLTVLHQDWLNGKEY | |
| | | KCKVSNKGLPSSIEKTISKAKGQPREPQVYTLPPSQEEMTK | |
| | | NQVSLTCLVKGFYPSDIAVEWESNGQPENNYKTTPPVLDS | |
| | | DGSFFLYSRLTVDKSRWQEGNVFSCSVMHEALHNHYTQK | |
| | | SLSLSLGKGGGGSGGGGSGGGGSGGGGSHSHRDFQPVLH | |
| | | LVALNSPLSGGMRGIRGADFQCFQQARAVGLAGTFRAFLS | |
| | | SRLQDLYSIVRRADRAAVPIVNLKDELLFPSWEALFSGSEG | |
| | | PLKPGARIFSFDGKDVLRHPTWPQKSVWHGSDPNGRRLTE | |
| | | SYCETWRTEAPSATGQASSLLGGRLLGQSAASCHHAYIVL | |
| | | CIENSFMTASK | |
| Vector | C3i(N15Q)- | MEFGLSWLFLVAILKGVQCDKGICVWQDWGAHRCTQGG | 301 |
| KMR, | Fc4- | GGSGGGGSGGGGSDKRVESKYGPPCPPCPAPEFLGGPSVF | |
| Vector | C3i(N15Q | LFPPKPKDTLMISRTPEVTCVVVDVSQEDPEVQFNWYVDG | |
| KPT | | VEVHNAKTKPREEQFNSTYRVVSVLTVLHQDWLNGKEY | |
| | | KCKVSNKGLPSSIEKTISKAKGQPREPQVYTLPPSQEEMTK | |
| | | NQVSLTCLVKGFYPSDIAVEWESNGQPENNYKTTPPVLDS | |
| | | DGSFFLYSRLTVDKSRWQEGNVFSCSVMHEALHNHYTQK | |
| | | SLSLSLGKGGGGSGGGGSGGGGSGGGGSGICVWQDWGA | |
| | | HRCTQ | |
| Vector | C3i(N15Q)- | MEFGLSWLFLVAILKGVQCDKGICVWQDWGAHRCTQGG | 302 |
| KPC | Fc4 | GGSGGGGSGGGGSDKRVESKYGPPCPPCPAPEFLGGPSVF | |
| | | LFPPKPKDTLMISRTPEVTCVVVDVSQEDPEVQFNWYVDG | |
| | | VEVHNAKTKPREEQFNSTYRVVSVLTVLHQDWLNGKEY | |
| | | KCKVSNKGLPSSIEKTISKAKGQPREPQVYTLPPSQEEMTK | |
| | | NQVSLTCLVKGFYPSDIAVEWESNGQPENNYKTTPPVLDS | |
| | | DGSFFLYSRLTVDKSRWQEGNVFSCSVMHEALHNHYTQK | |
| | | SLSLSLGK | |

### Example 9. Optimization of AAV Constructs and Expression

AAV2.N54 was demonstrated optimal for delivering GOIs into retinal pigment epithelium (RPE) cells. Sf9 rhabdovirus (Sf-RhV) free Sf9 cells were used for AAV production. The cells were cultured at 28° C. and 150 RPM in ESF AF medium (Expression Systems). The cells were split 1:4 once the cell density reaches 7×10⁶ cells/ml for maintenance. Recombinant baculovirus (rBVs) were generated according to Invitrogen's protocol (Carlsbad, CA). Briefly, the constructed plasmids were transformed into ΔcathDH10Bac competent E. coli cells to generate recombinant bacmid DNA. The bacmid DNA was transfected into Sf-RhV free Sf9 cells to generate rBVs carrying the GOIs. The rBVs were amplified and quantified by QPCR. Sf-RhV free Sf9 cells were co-infected with rBV-RepCap and rBV-GOI to generate AAV vectors.

Sf-RhV free Sf9 cells were cultured to 9±2×10⁶ cells/ml and diluted 1:1 with fresh ESF AF media. ~400 virus per cell of rBV containing the designated rep-cap genes and ~400 virus per cell of rBV containing IgG4-Fc fusion genes were used to co-infect the Sf-RhV free Sf9 cells for 3 days at 28° C. in a shaker incubator with an agitation of 180 RPM. The infected Sf9 cells were harvested by centrifugation at 3,000 RPM for 10 min. Cell pellets were lysed in Sf9 lysis buffer (50 mM Tris-HCl, pH7.8, 2 mM MgCl2, 1% Sarkosyl, 1% Triton X-100, and 140 units/ml Benzonase®, Millipore, Burlington, MA) at 37° C. with vigorous shaking at 300 RPM for one hour. At the end of incubation, 5 M NaCl was added to adjust the salt concentration of the lysate to about 1M to further dissociate the AAV vectors from cell matrix. Cell debris was removed by centrifugation at 8,000 RPM for 20 min. The cleared lysates were loaded onto CsCl step-gradient and subjected to ultracentrifugation at 28,000 RPM for 20 hours in swing bucket rotors. The viral bands were withdrawn through a syringe with an 18-gauge needle and loaded onto a second CsCl and subjected to linear-ultracentrifugation at 65,000 RPM for 20 hours. The full AAV band was withdrawn and passed through two PD-10 desalting columns (GE HealthCare) to remove the CsCl and detergents and at the same time exchanged to Formulation Buffer (10 mM Sodium phosphate, 180 mM NaCl, and 0.001% Pluronic F-68). To obtain AAVs with higher purity and safety for animal studies, selected AAV vectors after in vitro screening were also underwent AAVx affinity column and XQ anion exchange column before buffer exchange into Formulation Buffer. Digit droplet PCR (ddPCR) was performed to determine the AAV vector genome copy numbers with primers/probes targeting GOI.

Constructs packaged by AAV2-N54 capsid were produced for delivering various forms of Fc fusion genes and/or sCD59 gene. All the AAVs prepared for this study were listed in the Table 28 with the AAV titer, amount and yields. The ORF sequences of GOI and their encoded protein sequences are listed in Table 30 and Table 31, respectively.

TABLE 28
Yields of AAV vectors determined with ITR-QPCR

| | | | Total | | |
|---|---|---|---|---|---|
| | | AAV | AAV | Total | |
| | | titer | Vol | Yield | Yield |
| Construct | Lot # | (vg/mL) | (mL) | (vg) | (vg/L) |
| Packaged | | | | | |
| in Vector | | | | | |
| TKG | | | | | |
| Vector | 23-029 | 1.97E13 | 2.6 | 5.12E13 | 2.56E14 |
| GTP | | | | | |
| Vector | 23-038 | 1.69E13 | 2.3 | 3.89E13 | 1.94E14 |
| GAM | | | | | |
| Vector | 23-037 | 5.48E12 | 2.8 | 1.53E13 | 7.67E13 |
| GEM | | | | | |
| Vector | 23-046, | 2.00E13 | 1.6 | 3.20E13 | 1.60E14 |
| GGE | AAVx | | | | |
| Vector | 23-047, | 2.00E13 | 1 | 2.00E13 | 1.00E14 |
| GGG | AAVx | | | | |
| Vector | 23-048 | 3.68E13 | 2.3 | 8.46E13 | 4.23E14 |
| GGK | | | | | |
| Vector | 23-063, | 2.07E13 | 1.3 | 2.69E13 | 1.35E14 |
| GGM | AAVx | | | | |
| Vector | 23-084 | 8.88E12 | 2.6 | 2.31E13 | 1.15E14 |
| GGP | | | | | |
| Vector | 23-049 | 2.40E13 | 2.3 | 5.52E13 | 2.76E14 |
| GGQ | | | | | |
| Vector | 23-075 | 2.46E12 | 2.3 | 5.66E12 | 2.83E13 |
| GGR | | | | | |
| Vector | 23-050 | 3.09E13 | 3.2 | 9.89E13 | 4.94E14 |
| GKT | | | | | |
| Vector | 23-085 | 8.16E12 | 2.6 | 2.12E13 | 1.06E14 |
| GKA | | | | | |
| Vector | 23-086, | 1.81E13 | 0.6 | 1.09E13 | 5.43E13 |
| GKC | AAVx | | | | |
| Vector | 23-087, | 1.85E13 | 0.14 | 2.59E12 | 1.30E13 |
| GKK | AAVx | | | | |
| Vector | 23-057 | 3.68E13 | 2.3 | 8.46E13 | 4.23E14 |
| GKM | | | | | |
| Vector | 23-058 | 4.23E12 | 2.1 | 8.88E12 | 4.44E13 |
| GKP | | | | | |
| Vector | 23-059 | 2.39E13 | 2.3 | 5.50E13 | 2.75E14 |
| GKQ | | | | | |
| Vector | 23-060 | 3.30E13 | 2.3 | 7.59E13 | 3.80E14 |
| GKR | | | | | |
| Vector | 23-070 | 3.60E13 | 1.3 | 4.68E13 | 2.34E14 |
| GMA | | | | | |
| Vector | 23-071 | 2.11E13 | 3.3 | 6.96E13 | 3.48E14 |
| GMC | | | | | |
| Vector | 23-079 | 2.97E13 | 3.3 | 9.80E13 | 4.90E14 |
| GME | | | | | |
| Vector | 23-080 | 5.32E12 | 2.3 | 1.22E13 | 6.13E13 |
| GMG | | | | | |
| Vector | 23-081 | 3.30E13 | 3.3 | 1.09E14 | 5.45E14 |
| GMK | | | | | |
| Vector | 23-082 | 3.85E13 | 2.3 | 8.86E13 | 4.43E14 |
| GMM | | | | | |
| Vector | 23-083 | 2.53E13 | 3.3 | 8.35E13 | 4.17E14 |
| GMP | | | | | |
| Vector | 23-077 | 2.76E13 | 3.3 | 9.11E13 | 4.55E14 |
| GMQ | | | | | |
| Vector | 23-078 | 2.16E13 | 3.2 | 6.91E13 | 3.46E14 |
| GMR | | | | | |
| Vector | 23-088, | 1.86E13 | 0.8 | 1.49E13 | 7.44E13 |
| KAQ | AAVx | | | | |
| Vector | 23-089, | 1.74E13 | 0.4 | 6.96E12 | 3.48E13 |
| KAR | AAVx | | | | |
| Vector | 24-013 | 3.74E11 | 1.5 | 5.61E11 | 2.81E12 |
| KAG | | | | | |
| Vector | 24-014 | 6.76E12 | 2.2 | 1.49E13 | 7.44E13 |
| KGQ | | | | | |
| Vector | 24-016 | 1.20E13 | 2.3 | 2.76E13 | 1.38E14 |
| KKT | | | | | |
| Vector | 24-017 | 1.09E13 | 2.8 | 3.05E13 | 1.53E14 |
| KKA | | | | | |
| Vector | 24-027 | 9.40E12 | 1.5 | 1.41E13 | 7.05E13 |
| KGR | | | | | |
| Packaged | | | | | |
| in Vector | | | | | |
| CRG | | | | | |
| Vector | 24-030 | 7.57E12 | 1.8 | 1.36E13 | 9.08E13 |
| KKT | | | | | |
| Vector | 24-032, | 2.28E12 | 9.5 | 2.17E13 | 1.20E13 |
| KKT | AAVx, XQ | | | | |
| Vector | 24-031 | 9.57E12 | 2.2 | 2.11E13 | 1.40E14 |
| KKA | | | | | |
| Vector | 24-038, | 1.36E13 | 15.2 | 2.07E14 | 3.45E14 |
| KKA | AAVx, XQ | | | | |
| Vector | 24-057 | 2.87E12 | 1.5 | 4.31E12 | 1.44E13 |
| KMR | | | | | |
| Vector | 24-063, | 1.22E13 | 12.6 | 1.54E14 | 7.69E13 |
| KMR | AAVx, XQ | | | | |
| Vector | 24-049, | 1.71E12 | 6.2 | 1.06E13 | 3.53E13 |
| KPT | AAVx, XQ | | | | |
| Vector | 24-050, | 1.49E12 | 6.5 | 9.69E12 | 3.23E13 |
| KPA | AAVx, XQ | | | | |
| Vector | 24-051, | 3.55E12 | 6.5 | 2.31E13 | 7.69E13 |
| KPC | AAVx, XQ | | | | |
| Vector | 24-058 | 4.41E12 | 1.5 | 6.62E12 | 2.21E13 |
| KPP | | | | | |
| Vector | 24-064, | 1.87E13 | 9.6 | 1.80E14 | 8.98E13 |
| KPP | AAVx, XQ | | | | |
| Vector | 24-060 | 2.10E13 | 13.8 | 2.90E14 | 4.83E14 |
| KPQ | | | | | |
| Vector | 24-066 (cell | N.D. | N.D. | N.D. | N.D. |
| KPR | pellet at | | | | |
| | −80 C.) | | | | |
| Vector | 24-069 (in | N.D. | N.D. | N.D. | N.D. |
| KQT | purification) | | | | |
| Vector | 24-070 (in | N.D. | N.D. | N.D. | N.D. |
| KQA | purification) | | | | |

N.D. = No Data

### Optimization of AAV Constructs and Expression (SP)

The selected AAV constructs are listed in the Table 28. This study focused on the constructs with two or more GOIs, and those with single GOI are used for protein expression comparison. The two or more GOIs in a AAV construct were distributed into dual cassettes or linked together under a 2A system. In the single cassette, the sCD59 gene was placed in a single cassette under CBA or sCAG promoter, and the C3i fusion genes are in another cassette driven by CBA promoter. In the 2A systems, the sCD59 is immediately under CBA or sCAG promoter and then linked with C3i fusion genes by P2A-T2A.

Expression of AAV constructs were performed by developing four independent ELISAs for each of the gene products (sCD59, C3i, CNP and Endostatin).

In order to determine the expression in HEK293 LTV and ARPE-19 cells, the cells were transduced by AAV constructs using standard protocol. Five days after transductions, spent medium was collected and expression was determined by the specific ELISAs developed in-house. All the ELISAs were performed on 96-well maxisorp plates. Table 51 provides the list of antibodies used for coating and detection along with the standard used in the assays. All the plates were coated with 5 μg/mL of coating antibody at 2-8° C. overnight. The plates were washed thrice with wash buffer and blocked using 1% casein in PBS with tween 20. After blocking, samples and standards were added to the plate and incubated for 1-2 hrs. After washing for 3 times, detection antibody was added and incubated for 1 hr. After streptavidin-HRP, the plates were developed using TMB substrate. The absorbance was read at 450 nm with a reference wavelength of 600 nm.

TABLE 51
ELISA procedure for Example 9

| | Coating | | | |
|---|---|---|---|---|
| | antibody | Standard | Detection | Streptavidin- |
| ELISA | (5 μg/mL) | (ng/ml) | antibody/dilution | HRP/dilution |
| sCD59 | Mouse anti- | sCD59 or | Mouse anti-CD59- | Yes/1:10,000 |
| | CD59 | commercial CD59 | Biotin/1:5000 | |
| | | (50-0.8) | | |
| C3i | Goat anti- | C3i-Fc or C3i-Fc- | Mouse anti-human | No |
| | human IgG | CNP or C3i-Fc- | IgG-HRP | |
| | Fc | Endostatin (50-0.8) | | |
| Fc-CNP | Rat anti- | Fc-CNP or C3i-Fc- | Goat anti-human | Yes/1:10,000 |
| | CNP | CNP (25-0.4) | IgG-Biotin/1:10,000 | |
| Endostatin | Mouse anti- | Commercial | Mouse anti- | No |
| | endostatin | Endostatin (3-0.05) | endostatin-HRP | |

Table 52 shows the protein expression of selected AAVs in ARPE-19 cells. When the sCD59 gene and C3i fusion genes were cloned into dual cassettes or 2A system, the sCD59 protein expression showed no difference, but the C3i fusion proteins expression in dual cassettes were much higher than 2A system, for example, Vector KAQ vs. Vector KAR, Vector KCA vs. Vector KCC, Vector KEG vs. Vector KEK, Vector KCP vs. Vector KCQ, Vector KKA vs. Vector KEE. In general, sCD59 driven by CBA promoter had higher expression than by sCAG promoter. In addition, when the WPRE was introduced between sCD59 gene and the poly A signal, it enhanced the sCD59 gene expression significantly. For example, compared with Vector KKA, introduction of WPRE into Vector KPP increased sCD59 expression by about 16 times.

TABLE 52
AAV expression in ARPE19 cells

| | | | | | | | C3i- | C3i- |
|---|---|---|---|---|---|---|---|---|
| | | | | | | | Fc4- | Fc4- |
| | | CBA/CAG | | | CD59 | C3i | CNP36 | Endostatin |
| Construct | Description | for CD59 | Dual/2A | WPRE | (μg/mL) | (μg/mL) | (μg/mL) | (μg/mL) |
| Vector | CBA- | CBA | Dual | No | 0.2 ± 0.0 | N.A. | 5.7 ± 0.1 | N.A. |
| KAQ | sCD59- | | | | | | | |
| | CBA-DK | | | | | | | |
| | C3i-Fc4- | | | | | | | |
| | CNP36 | | | | | | | |
| Vector | CBA- | CBA | 2A | No | 0.2 | N.A. | 0.2 | N.A. |
| KAR | sCD59- | | | | | | | |
| | Furin- | | | | | | | |
| | GSG-P2A- | | | | | | | |
| | T2A-DK | | | | | | | |
| | C3i-Fc4- | | | | | | | |
| | 4xGGGs- | | | | | | | |
| | CNP36 | | | | | | | |
| Vector | sCAG- | CAG | Dual | No | 0.2 ± 0.0 | N.A. | 5.4 ± 0.8 | N.A. |
| KCA | sCD59- | | | | | | | |
| | CBA-DK | | | | | | | |
| | C3i-Fc4- | | | | | | | |
| | CNP36 | | | | | | | |
| Vector | sCAG- | CAG | 2A | No | 0.3 | N.A. | 0.3 | N.A. |
| KCC | sCD59- | | | | | | | |
| | Furin- | | | | | | | |
| | GSG-P2A- | | | | | | | |
| | T2A-DK | | | | | | | |
| | C3i-Fc4- | | | | | | | |
| | 4xGGGs- | | | | | | | |
| | CNP36 | | | | | | | |
| Vector | CBA- | CBA | Dual | Yes | 1.6 ± 1.4 | N.A. | 2.8 ± 1.8 | N.A. |
| KEQ | sCD59 | | | | | | | |
| | WPRE- | | | | | | | |
| | CBA-DK | | | | | | | |
| | C3i-Fc4- | | | | | | | |
| | CNP36 | | | | | | | |
| Vector | sCAG- | CAG | Dual | Yes | 1.0 ± 0.7 | N.A. | 4.6 ± 3.3 | N.A. |
| KGT | sCD59 | | | | | | | |
| | WPRE- | | | | | | | |
| | CBA-DK | | | | | | | |
| | C3i-Fc4- | | | | | | | |
| | CNP36 | | | | | | | |
| Vector | sCAG- | CAG | Dual | Yes | 2.5 ± 0.2 | N.A. | 4.4 ± 1.3 | N.A. |
| KKT | sCD59 | | | | | | | |
| | WPRE- | | | | | | | |
| | CBA-DK | | | | | | | |
| | C3i-Fc4- | | | | | | | |
| | CNP36 | | | | | | | |
| Vector | (CBA) DK | CBA | Single | No | N.A. | N.A. | 4.0 ± 2.1 | N.A. |
| GGE | C3i-Fc4- | | | | | | | |
| | CNP36 | | | | | | | |
| Vector | (sCAG) | CAG | Single | No | N.A. | N.A. | 2.2 ± 1.6 | N.A. |
| GGQ | DK C3i- | | | | | | | |
| | Fc4- | | | | | | | |
| | CNP36 | | | | | | | |
| Vector | CBA-Fc4- | CBA | Single | No | N.A. | N.A. | 22.7 | N.A. |
| CPE | CNP36 | | | | | | | |
| Vector | sCAG- | CAG | Dual | No | 0.2 ± 0.1 | 15.0 ± 7.5 | N.A. | N.A. |
| KEG | sCD59- | | | | | | | |
| | (CBA)- | | | | | | | |
| | Fc4-C3i | | | | | | | |
| Vector | sCAG- | CAG | 2A | No | 0.3 | 0.2 | N.A. | N.A. |
| KEK | sCD59- | | | | | | | |
| | Furin- | | | | | | | |
| | GSG-P2A- | | | | | | | |
| | T2A-Fc4- | | | | | | | |
| | 4xGGGS- | | | | | | | |
| | C3i | | | | | | | |
| Vector | CBA- | CBA | Dual | No | 0.6 ± 0.2 | 11.3 ± 2.5 | N.A. | N.A. |
| KCP | sCD59- | | | | | | | |
| | (CBA)- | | | | | | | |
| | Fc4-C3i | | | | | | | |
| Vector | CBA- | CBA | 2A | No | 0.3 ± 0.2 | 0.2 ± 0.0 | N.A. | N.A. |
| KCQ | sCD59- | | | | | | | |
| | Furin- | | | | | | | |
| | GSG-P2A- | | | | | | | |
| | T2A-Fc4- | | | | | | | |
| | 4xGGGS- | | | | | | | |
| | C3i | | | | | | | |
| Vector | CBA- | CBA | Dual | Yes | 2.1 ± 1.2 | 11.0 ± 2.6 | N.A. | N.A. |
| KER | sCD59 | | | | | | | |
| | WPRE- | | | | | | | |
| | (CBA)- | | | | | | | |
| | Fc4-C3i | | | | | | | |
| Vector | CBA- | CBA | Dual | Yes | 1.0 ± 0.3 | 6.2 ± 5.8 | N.A. | N.A. |
| KGQ | sCD59 | | | | | | | |
| | WPRE- | | | | | | | |
| | (CBA)- | | | | | | | |
| | Fc4-C3i | | | | | | | |
| Vector | sCAG- | CAG | Dual | Yes | 1.1 ± 0.8 | 6.6 ± 4.8 | N.A. | N.A. |
| KGA | sCD59 | | | | | | | |
| | WPRE- | | | | | | | |
| | (CBA)- | | | | | | | |
| | Fc4-C3i | | | | | | | |
| Vector | sCAG- | CAG | Dual | Yes | 0.9 ± 0.1 | 2.9 ± 0.8 | N.A. | N.A. |
| KGR | sCD59 | | | | | | | |
| | WPRE- | | | | | | | |
| | (CBA)- | | | | | | | |
| | Fc4-C3i | | | | | | | |
| Vector | CBA- | CBA | Dual | Yes | 6.3 ± 0.5 | 2.5 ± 0.1 | N.A. | N.A. |
| KMR | cCD59 | | | | | | | |
| | WPRE- | | | | | | | |
| | (CBA)- | | | | | | | |
| | C3i-Fc4- | | | | | | | |
| | C3i | | | | | | | |
| Vector | (CBA) | CBA | Single | No | N.A. | 4.9 ± 1.7 | N.A. | N.A. |
| GGG | Fc4-C3i | | | | | | | |
| Vector | (CBA) | CBA | Single | No | N.A. | 15.5 | N.A. | N.A. |
| KAG | Fc4-C3i | | | | | | | |
| Vector | sCAG- | CAG | Dual | No | 0.3 ± 0.2 | N.A. | N.A. | 16.2 ± 11.6 |
| KEC | sCD69- | | | | | | | |
| | CBA-DK | | | | | | | |
| | C3i-Fc4- | | | | | | | |
| | endostatin | | | | | | | |
| Vector | sCAG- | CAG | Dual | No | 0.1 ± 0.0 | N.A. | N.A. | 14.2 ± 6.4 |
| KKA | sCD69- | | | | | | | |
| | CBA-DK | | | | | | | |
| | C3i-Fc4- | | | | | | | |
| | endostatin | | | | | | | |
| Vector | sCAG- | CAG | 2A | No | 0.2 ± 0.1 | N.A. | N.A. | 4.2 ± 2.1 |
| KEE | sCD69- | | | | | | | |
| | Furin- | | | | | | | |
| | GSG-P2A- | | | | | | | |
| | T2A-DK | | | | | | | |
| | C3i-Fc4- | | | | | | | |
| | endostatin | | | | | | | |
| Vector | sCAG- | CAG | Dual | Yes | 0.3 ± 0.1 | N.A. | N.A. | 16.0 ± 4.8 |
| KGC | sCD69 | | | | | | | |
| | WPRE- | | | | | | | |
| | CBA-DK | | | | | | | |
| | C3i-Fc4- | | | | | | | |
| | endostatin | | | | | | | |
| Vector | sCAG- | CAG | Dual | Yes | 1.6 ± 0.7 | N.A. | N.A. | 2.8 ± 0.2 |
| KPP | sCD69 | | | | | | | |
| | WPRE- | | | | | | | |
| | CBA-DK | | | | | | | |
| | C3i-Fc4- | | | | | | | |
| | endostatin | | | | | | | |
| Vector | (CBA)DK | CBA | Single | No | N.A. | N.A. | N.A. | 11.7 ± 3.8 |
| GKC | C3i-Fc4- | | | | | | | |
| | endostatin | | | | | | | |
| Vector | (sCAG)DK | CAG | Single | No | N.A. | N.A. | N.A. | 5.9 ± 2.8 |
| KEM | C3i-Fc4- | | | | | | | |
| | endostatin | | | | | | | |
| Vector | (CBA) | CBA | Single | No | 3.3 ± 0.3 | N.A. | N.A. | N.A. |
| GEM | sCD59snap | | | | | | | |
| | gene | | | | | | | |
| Vector | (sCAG) | CAG | Single | No | 2.1 ± 0.1 | N.A. | N.A. | N.A. |
| GKT | sCD59snap | | | | | | | |
| | gene | | | | | | | |

N.A. = Not applicable

### Three Candidate Constructs for Monkey Study

With a broad screening on AAV constructs completed, the GOI expressions of each construct were displayed. Based on the AAV expression in vitro, in the combination with information on Fc-fusion protein binding affinity and IC50 value, 3 candidate constructs, Vector KMR, Vector KKT, and Vector KPP, were selected for monkey study (FIG. 24). All these three constructs have sCD59 under with CBA or sCAG promoter in the first cassette, but they have different GOI in the second cassette. For example, Vector KMR, Vector KKT, and Vector KPP has C3i-Fc4-C3i, C3i-Fc4-CNP36, and C3i-Fc4-endostatin, respectively.

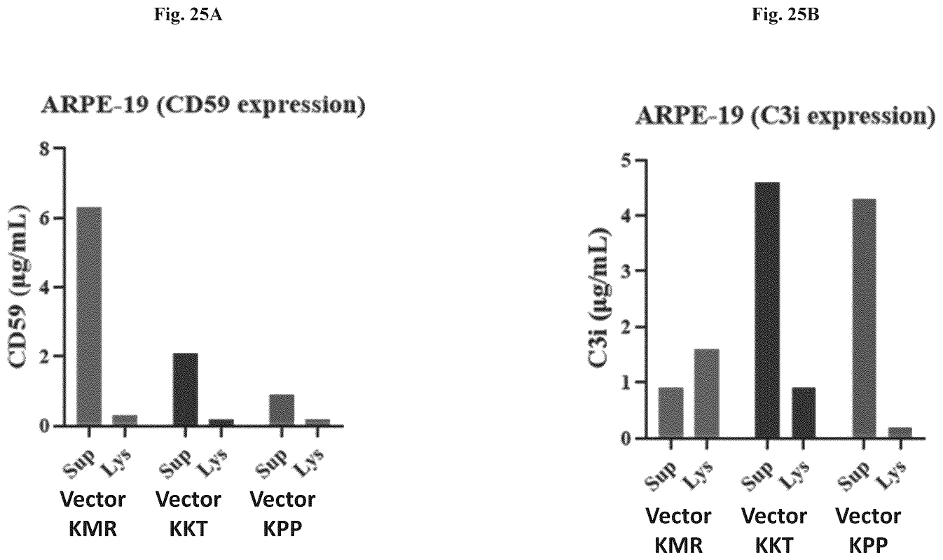

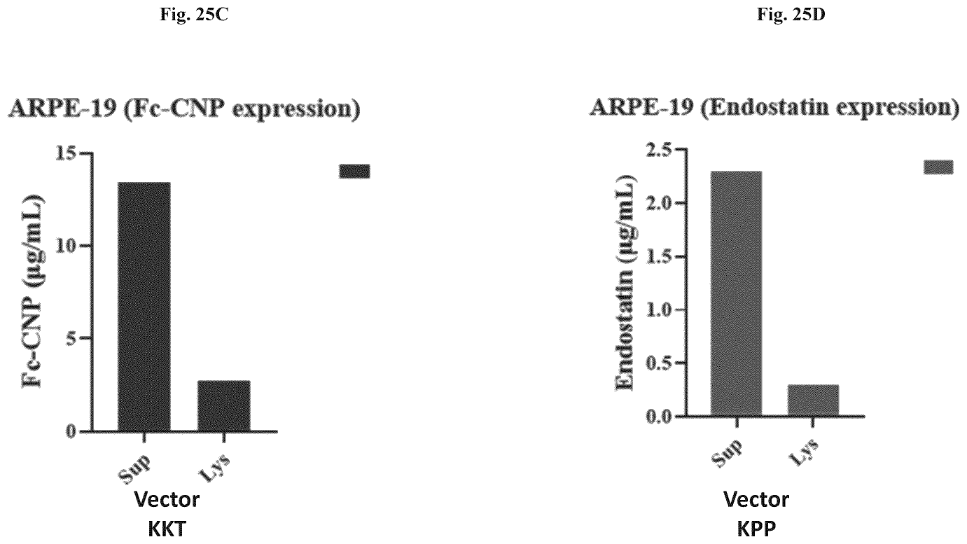

Their AAV expressions in the HEK293LTV and ARPE-19 cells were summarized in Table 29 and Table 30, respectively. Vector KMR had the highest sCD59 expression due to the CBA promoter and Vector KPP had the least sCD59 expression (FIG. 25A) due to the construct having only WPRE in the first cassette. In the case of IgG4-Fc fusion protein, Vector KMR had the lowest expression of C3i-Fc4-C3i and Vector KKT and Vector KPP expressed the similar amount of Fc4 fusion proteins (FIG. 25B). In addition, Vector KKT expressed Fc-CNP (FIG. 25C) and Vector KPP expressed endostatin (FIG. 25D). All proteins were efficiently secreted outside the cells with more than 80% of protein present in the medium, except for C3i-Fc4-C3i.

TABLE 29
AAV expression of sCD59 and C3i fusion protein in HEK293LTV cells

| | sCD59 | | | | | C3i | | | | |
|---|---|---|---|---|---|---|---|---|---|---|
| | ug/mL | | | % expression | | ug/mL | | | % expression | |
| HEK293 LTV | Sup | Lys | Total | Sup | Lys | Sup | Lys | Total | Sup | Lys |
| 294-Vector | 3.8 | 0.4 | 4.2 | 90.6 | 9.4 | 1.5 | 1.5 | 3 | 50.4 | 49.6 |
| KMR | | | | | | | | | | |
| 294-Vector | 1.5 | 0.3 | 1.8 | 85.9 | 14.1 | 9.4 | 1.2 | 10.6 | 88.6 | 11.4 |
| KKT | | | | | | | | | | |
| 294-Vector | 0.7 | 0.1 | 0.9 | 84.7 | 15.3 | 5 | 0.7 | 5.7 | 87.8 | 12.2 |
| KPP | | | | | | | | | | |
| | CNP | | | | | Endostatin | | | | |
| | ug/mL | | | % expression | | ug/mL | | | % expression | |
| HEK293 LTV | Sup | Lys | Total | Sup | Lys | Sup | Lys | Total | Sup | Lys |
| 294-Vector | N.A. | N.A. | N.A. | N.A. | N.A. | N.A. | N.A. | N.A. | N.A. | N.A. |
| KMR | | | | | | | | | | |
| 294-Vector | 13.1 | 2.4 | 15.5 | 84.7 | 15.3 | N.A. | N.A. | N.A. | N.A. | N.A. |
| KKT | | | | | | | | | | |
| 294-Vector | N.A. | N.A. | N.A. | N.A. | N.A. | 3.6 | 0.4 | 4.1 | 89.3 | 10.7 |
| KPP | | | | | | | | | | |

N.A. = Not Applicable

TABLE 30
AAV expression of sCD59 and C3i fusion protein in ARPE-19 cells

| | sCD59 | | | | | C3i | | | | |
|---|---|---|---|---|---|---|---|---|---|---|
| | ug/mL | | | % expression | | ug/mL | | | % expression | |
| ARPE-19 | Sup | Lys | Total | Sup | Lys | Sup | Lys | Total | Sup | Lys |
| 294-Vector | 6.3 | 0.3 | 6.6 | 96 | 4 | 0.9 | 1.6 | 2.5 | 35.4 | 64.6 |
| KMR | | | | | | | | | | |
| 294-Vector | 2.1 | 0.2 | 2.3 | 91.1 | 8.9 | 4.6 | 0.9 | 5.5 | 83.6 | 16.4 |
| KKT | | | | | | | | | | |
| 294-Vector | 0.9 | 0.2 | 1.1 | 82.4 | 17.6 | 4.3 | 0.2 | 4.6 | 95 | 5 |
| KPP | | | | | | | | | | |
| | CNP | | | | | Endostatin | | | | |
| | ug/mL | | | % expression | | ug/mL | | | % expression | |
| ARPE-19 | Sup | Lys | Total | Sup | Lys | Sup | Lys | Total | Sup | Lys |
| 294-Vector | N.A. | N.A. | N.A. | N.A. | N.A. | N.A. | N.A. | N.A. | N.A. | N.A. |
| KMR | | | | | | | | | | |
| 294-Vector | 13.4 | 2.7 | 16.1 | 83.5 | 16.5 | N.A. | N.A. | N.A. | N.A. | N.A. |
| KKT | | | | | | | | | | |
| 294-Vector | N.A. | N.A. | N.A. | N.A. | N.A. | 2.3 | 0.3 | 2.6 | 90.1 | 9.9 |
| KPP | | | | | | | | | | |

N.A. = Not Applicable

TABLE 31
DNA sequences of plasmids used in Example 9

| Vector | | of plasmids used in Example | |
|---|---|---|---|
| name | Protein | Nucleic acid sequence (ORF for GOI) | SEQ ID NO |
| Vector | sCD59 | ATGGGCATCCAGGGCGGCAGCGTGCTGTTCGGCCTGCTG | 270 |
| KGQ, | | CTGGTGCTGGCCGTGTTCTGCCACAGCGGCCACAGCCTGC | |
| Vector | | AGTGCTACAACTGCCCCAACCCCACCGCCGACTGCAAGA | |
| KGR, | | CCGCCGTGAACTGCAGCAGCGACTTCGACGCCTGCCTGA | |
| Vector | | TCACCAAGGCCGGCCTGCAGGTGTACAACAAGTGCTGGA | |
| KKT, | | AGTTCGAGCACTGCAACTTCAACGACGTGACCACCAGGC | |
| Vector | | TGAGGGAGAACGAGCTGACCTACTACTGCTGCAAGAAGG | |
| KKA. | | ACCTGTGCAACTTCAACGAGCAGCTGGAGAACTAA | |
| Vector | | | |
| KMR, | | | |
| Vector | | | |
| KPT, | | | |
| Vector | | | |
| KPC, | | | |
| Vector | | | |
| KPP | | | |
| Vector | C3i(N15 | ATGGAGTTCGGCCTGAGCTGGCTGTTCCTGGTGGCCATCC | 272 |
| KKT | Q)-Fc4- | TTAAGGGCGTGCAGTGCGACAAGGGCATCTGCGTGTGGC | |
| | CNP36 | AGGACTGGGGCGCCCACCGCTGCACCCAGGGCGGAGGCG | |
| | | GAAGCGGCGGAGGCGGATCTGGCGGAGGCGGAAGCGAC | |
| | | AAGAGAGTGGAGAGCAAGTACGGCCCCCCCTGCCCCCCC | |
| | | TGCCCCGCCCCCGAGTTCCTGGGCGGCCCCAGCGTGTTCC | |
| | | TGTTCCCCCCCAAGCCCAAGGACACCCTGATGATCAGCA | |
| | | GAACCCCCGAGGTGACCTGCGTGGTGGTGGACGTGAGCC | |
| | | AGGAGGACCCCGAGGTGCAGTTCAACTGGTACGTGGACG | |
| | | GCGTGGAGGTGCACAACGCCAAGACCAAGCCCAGAGAG | |
| | | GAGCAGTTCAACAGCACCTACAGAGTGGTGAGCGTGCTG | |
| | | ACCGTGCTGCACCAGGACTGGCTGAACGGCAAGGAGTAC | |
| | | AAGTGCAAGGTGAGCAACAAGGGCCTGCCCAGCAGCATC | |
| | | GAGAAGACCATCAGCAAGGCCAAGGGCCAGCCCAGAGA | |
| | | GCCCCAGGTGTACACCCTGCCCCCCAGCCAGGAGGAGAT | |
| | | GACCAAGAACCAGGTGAGCCTGACCTGCCTGGTGAAGGG | |
| | | CTTCTACCCCAGCGACATCGCCGTGGAGTGGGAGAGCAA | |
| | | CGGCCAGCCCGAGAACAACTACAAGACCACCCCCCCCGT | |
| | | GCTGGACAGCGACGGCAGCTTCTTCCTGTACAGCAGACT | |
| | | GACCGTGGACAAGAGCAGATGGCAGGAGGGCAACGTGTT | |
| | | CAGCTGCAGCGTGATGCACGAGGCCCTGCACAACCACTA | |
| | | CACCCAGAAGAGCCTGAGCCTGAGCCTGGGCAAGGGCGG | |
| | | AGGCGGAAGCGGCGGAGGCGGATCTGGCGGAGGCGGAA | |
| | | GCGGCGGAGGCGGCTCTGAGCACCCCAACGCGCGCAAAT | |
| | | ACAAAGGAGCCAACAAGAAGGGCTTGTCCAAGGGCTGCT | |
| | | TCGGCCTCAAGCTGGACCGAATCGGCTCCATGAGCGGCC | |
| | | TGGGATGTTAG | |
| Vector | C3i(N15 | ATGGAGTTCGGCCTGAGCTGGCTGTTCCTGGTGGCCATCC | 274 |
| KMR, | Q)-Fc4- | TTAAGGGCGTGCAGTGCGACAAGGGCATCTGCGTGTGGC | |
| Vector | C3i(N15Q) | AGGACTGGGGCGCCCACCGCTGCACCCAGGGCGGAGGCG | |
| KPT | | GAAGCGGCGGAGGCGGATCTGGCGGAGGCGGAAGCGAC | |
| | | AAGAGAGTGGAGAGCAAGTACGGCCCCCCCTGCCCCCCC | |
| | | TGCCCCGCCCCCGAGTTCCTGGGCGGCCCCAGCGTGTTCC | |
| | | TGTTCCCCCCCAAGCCCAAGGACACCCTGATGATCAGCA | |
| | | GAACCCCCGAGGTGACCTGCGTGGTGGTGGACGTGAGCC | |
| | | AGGAGGACCCCGAGGTGCAGTTCAACTGGTACGTGGACG | |
| | | GCGTGGAGGTGCACAACGCCAAGACCAAGCCCAGAGAG | |
| | | GAGCAGTTCAACAGCACCTACAGAGTGGTGAGCGTGCTG | |
| | | ACCGTGCTGCACCAGGACTGGCTGAACGGCAAGGAGTAC | |
| | | AAGTGCAAGGTGAGCAACAAGGGCCTGCCCAGCAGCATC | |
| | | GAGAAGACCATCAGCAAGGCCAAGGGCCAGCCCAGAGA | |
| | | GCCCCAGGTGTACACCCTGCCCCCCAGCCAGGAGGAGAT | |
| | | GACCAAGAACCAGGTGAGCCTGACCTGCCTGGTGAAGGG | |
| | | CTTCTACCCCAGCGACATCGCCGTGGAGTGGGAGAGCAA | |
| | | CGGCCAGCCCGAGAACAACTACAAGACCACCCCCCCCGT | |
| | | GCTGGACAGCGACGGCAGCTTCTTCCTGTACAGCAGACT | |
| | | GACCGTGGACAAGAGCAGATGGCAGGAGGGCAACGTGTT | |
| | | CAGCTGCAGCGTGATGCACGAGGCCCTGCACAACCACTA | |
| | | CACCCAGAAGAGCCTGAGCCTGAGCCTGGGCAAGGGCGG | |
| | | AGGCGGAAGCGGCGGAGGCGGATCTGGCGGAGGCGGAA | |
| | | GCGGCGGAGGCGGCTCTGGCATCTGCGTGTGGCAGGACT | |
| | | GGGGCGCCCACCGCTGCACCCAGTAA | |
| Vector | C3i(N15 | ATGGAGTTCGGCCTGAGCTGGCTGTTCCTGGTGGCCATCC | 276 |
| KPP | Q)-Fc4- | TTAAGGGCGTGCAGTGCGACAAGGGCATCTGCGTGTGGC | |
| | Endostatin | AGGACTGGGGCGCCCACCGCTGCACCCAGGGCGGAGGCG | |
| | | GAAGCGGCGGAGGCGGATCTGGCGGAGGCGGAAGCGAC | |
| | | AAGAGAGTGGAGAGCAAGTACGGCCCCCCCTGCCCCCCC | |
| | | TGCCCCGCCCCCGAGTTCCTGGGCGGCCCCAGCGTGTTCC | |
| | | TGTTCCCCCCCAAGCCCAAGGACACCCTGATGATCAGCA | |
| | | GAACCCCCGAGGTGACCTGCGTGGTGGTGGACGTGAGCC | |
| | | AGGAGGACCCCGAGGTGCAGTTCAACTGGTACGTGGACG | |
| | | GCGTGGAGGTGCACAACGCCAAGACCAAGCCCAGAGAG | |
| | | GAGCAGTTCAACAGCACCTACAGAGTGGTGAGCGTGCTG | |
| | | ACCGTGCTGCACCAGGACTGGCTGAACGGCAAGGAGTAC | |
| | | AAGTGCAAGGTGAGCAACAAGGGCCTGCCCAGCAGCATC | |
| | | GAGAAGACCATCAGCAAGGCCAAGGGCCAGCCCAGAGA | |
| | | GCCCCAGGTGTACACCCTGCCCCCCAGCCAGGAGGAGAT | |
| | | GACCAAGAACCAGGTGAGCCTGACCTGCCTGGTGAAGGG | |
| | | CTTCTACCCCAGCGACATCGCCGTGGAGTGGGAGAGCAA | |
| | | CGGCCAGCCCGAGAACAACTACAAGACCACCCCCCCCGT | |
| | | GCTGGACAGCGACGGCAGCTTCTTCCTGTACAGCAGACT | |
| | | GACCGTGGACAAGAGCAGATGGCAGGAGGGCAACGTGTT | |
| | | CAGCTGCAGCGTGATGCACGAGGCCCTGCACAACCACTA | |
| | | CACCCAGAAGAGCCTGAGCCTGAGCCTGGGCAAGGGCGG | |
| | | AGGCGGAAGCGGCGGAGGCGGATCTGGCGGAGGCGGAA | |
| | | GCGGCGGAGGCGGCTCTCACAGCCACAGGGACTTCCAGC | |
| | | CTGTGCTGCACCTGGTGGCCCTGAACAGCCCTCTGAGCGG | |
| | | AGGAATGAGGGGAATCAGGGGAGCCGACTTCCAGTGCTT | |
| | | CCAGCAGGCCAGGGCCGTGGGACTGGCCGGAACCTTCAG | |
| | | GGCCTTCCTGAGCAGCAGGCTGCAGGACCTGTACAGCAT | |
| | | CGTGAGGAGGGCCGACAGGGCCGCCGTGCCCATCGTGAA | |
| | | CCTGAAGGACGAGCTGCTGTTCCCTAGCTGGGAGGCCCT | |
| | | GTTCAGCGGAAGCGAGGGACCCCTGAAGCCCGGAGCCAG | |
| | | GATCTTCAGCTTCGACGGAAAGGACGTGCTGAGGCACCC | |
| | | TACCTGGCCTCAGAAGAGCGTGTGGCACGGAAGCGACCC | |
| | | TAACGGAAGGAGGCTGACCGAGAGCTACTGCGAGACCTG | |
| | | GAGGACCGAGGCCCCTAGCGCCACCGGCCAGGCCAGCAG | |
| | | CCTGCTGGGAGGAAGGCTGCTGGGACAGAGCGCCGCCAG | |
| | | CTGCCACCACGCCTACATCGTGCTGTGCATCGAGAACAG | |
| | | CTTCATGACCGCCAGCAAGTGA | |

TABLE 32
Peptides and fusion protein sequences of Example 9

| Vector | | | |
|---|---|---|---|
| name | Protein | Nucleic acid sequence (ORF for GOI) | SEQ ID NO |
| Vector | sCD59 | MGIQGGSVLFGLLLVLAVFCHSGHSLQCYNCPNPTADCKT | 297 |
| KGQ. | | AVNCSSDFDACLITKAGLQVYNKCWKFEHCNFNDVTTRLR | |
| Vector | | ENELTYYCCKKDLCNFNEQLEN | |
| KGR. | | | |
| Vector | | | |
| KKT | | | |
| Vector | | | |
| KKA, | | | |
| Vector | | | |
| KMR | | | |
| Vector | | | |
| KPT, | | | |
| Vector | | | |
| KPC. | | | |
| Vector | | | |
| KPP | | | |
| Vector | C3i(N15 | MEFGLSWLFLVAILKGVQCDKGICVWQDWGAHRCTQGGG | 299 |
| KKT | Q)-Fc4- | GSGGGGSGGGGSDKRVESKYGPPCPPCPAPEFLGGPSVFLF | |
| | CNP36 | PPKPKDTLMISRTPEVTCVVVDVSQEDPEVQFNWYVDGVE | |
| | | VHNAKTKPREEQFNSTYRVVSVLTVLHQDWLNGKEYKCK | |
| | | VSNKGLPSSIEKTISKAKGQPREPQVYTLPPSQEEMTKNQV | |
| | | SLTCLVKGFYPSDIAVEWESNGQPENNYKTTPPVLDSDGSF | |
| | | FLYSRLTVDKSRWQEGNVFSCSVMHEALHNHYTQKSLSLS | |
| | | LGKGGGGSGGGGSGGGGSGGGGSEHPNARKYKGANKKG | |
| | | LSKGCFGLKLDRIGSMSGLGC | |
| Vector | C3i(N15 | MEFGLSWLFLVAILKGVQCDKGICVWQDWGAHRCTQGGG | 301 |
| KMR. | Q)-Fc4- | GSGGGGSGGGGSDKRVESKYGPPCPPCPAPEFLGGPSVFLF | |
| Vector | C3i(N15Q) | PPKPKDTLMISRTPEVTCVVVDVSQEDPEVQFNWYVDGVE | |
| KPT | | VHNAKTKPREEQFNSTYRVVSVLTVLHQDWLNGKEYKCK | |
| | | VSNKGLPSSIEKTISKAKGQPREPQVYTLPPSQEEMTKNQV | |
| | | SLTCLVKGFYPSDIAVEWESNGQPENNYKTTPPVLDSDGSF | |
| | | FLYSRLTVDKSRWQEGNVFSCSVMHEALHNHYTQKSLSLS | |
| | | LGKGGGGSGGGGSGGGGSGGGGSGICVWQDWGAHRCTQ | |
| Vector | C3i(N15 | MEFGLSWLFLVAILKGVQCDKGICVWQDWGAHRCTQGGG | 303 |
| KPP | Q)-Fc4- | GSGGGGSGGGGSDKRVESKYGPPCPPCPAPEFLGGPSVFLF | |
| | Endostatin | PPKPKDTLMISRTPEVTCVVVDVSQEDPEVQFNWYVDGVE | |
| | | VHNAKTKPREEQFNSTYRVVSVLTVLHQDWLNGKEYKCK | |
| | | VSNKGLPSSIEKTISKAKGQPREPQVYTLPPSQEEMTKNQV | |
| | | SLTCLVKGFYPSDIAVEWESNGQPENNYKTTPPVLDSDGSF | |
| | | FLYSRLTVDKSRWQEGNVFSCSVMHEALHNHYTQKSLSLS | |
| | | LGKGGGGSGGGGSGGGGSGGGGSHSHRDFQPVLHLVALN | |
| | | SPLSGGMRGIRGADFQCFQQARAVGLAGTFRAFLSSRLQD | |
| | | LYSIVRRADRAAVPIVNLKDELLFPSWEALFSGSEGPLKPG | |
| | | ARIFSFDGKDVLRHPTWPQKSVWHGSDPNGRRLTESYCET | |
| | | WRTEAPSATGQASSLLGGRLLGQSAASCHHAYIVLCIENSF | |
| | | MTASK | |

### Example 10. In Vivo Expression Assay

Three AAV constructs were selected for mice intravitreal injection. The AAV-originated plasmids have dual cassettes. In the first cassette, all contained sCD59 with CBA or sCAG promoter in addition to a different expression elements. In the second cassette, they have different gene of interest, for example, Vector KGQ & Vector KGR, Vector KKT, and Vector KKA have Fc4-C3i, C3i-Fc4-CNP36, and C3i-Fc4-endostatin, respectively (FIG. 26). The ORF sequences of GOI and their encoded protein sequences are listed in Table 37 and Table 38, respectively

Twelve male mice were divided into 4 groups. Intravitreal (IVT) injection of Vector KGQ, Vector KGR, Vector KKT, and Vector KKA rAAV was performed for both eyes in each mouse, and 2.0 μL of rAAV was injected into each eye, resulting in the dosing of about 6×10⁹ vg per eye. The experimental design is shown in Table 33. No animal death occurred during or post-administration. All the animals were sacrificed on day 28 after IVT injection. Before both the eyes were isolated from each mouse, blood was collected and added with 10 μL of 50 mM EDTA and stored at −80° C. Table 34 shows the weights all the animals tested. Some of the homogenates were lost during the processing and are noted in the table below.

TABLE 33
Experimental design for mice intravitreal injection of rAAVs

| No. of | | Treatment | Dosage | Vol./ |
|---|---|---|---|---|
| animals | Group | (OU) | (vg/eye) | Route |
| 3 | 1 | Vector KGQ | 7.16E+09 | 2.0 μL/eye |
| 3 | 2 | Vector KGR | 6.22E+09 | |
| 3 | 3 | Vector KKT | 5.63E+09 | IVT |
| 3 | 4 | Vector KKA | 6.98E+09 | |

TABLE 34
The weight of dissected mice eyes after AAV injection

| | | Weight (mg) | |
|---|---|---|---|
| Group | Mouse # | OS | OD |
| 1 | 251 | 33.3 | 31.1 |
| | 252 | 28.8 | 28.8 |
| | 253 | 31.7 | 33* |
| 2 | 254 | 32.6 | 32.5 |
| | 255 | 31.8 | 28.9 |
| | 256 | 32.4 | 33.4 |
| 3 | 257 | 31.9 | 11.1* |
| | 258 | 32.2 | 30.5 |
| | 259 | 32.1 | 22.2 |
| 4 | 260 | 32.8 | 28.1 |
| | 261 | 29.9 | 30.2 |
| | 262 | 30.7 | 28.5 |

*Samples lost during the homogenization process

ELISA assays were performed according to the manufacturer protocol. For sCD59 ELISA, 5 μg/mL of mouse monoclonal anti-human CD59 Ab was coated on the plates. Standard CD59 range lies between 25-0.4 ng/mL. The bound CD59 proteins were detected with mouse monoclonal anti-human CD59 Ab (1:5000 dilution) and Streptavidin-HRP dilution (1:10,000 dilution). For C3i ELISA, 5 μg/mL of Goat anti-Human IgG Fc Cross-Adsorbed Secondary Antibody was coated on the plates. Vector KKT (C3i-Fc4-CNP36) protein was used as the standard (4.06 mg/mL), 250-3.9 ng/ml range of the assay. The bound C3i fusion proteins were detected with HRP-conjugated Mouse anti-Human IgG Fc monoclonal antibody (50B4A9) (with 1:1000 dilution). For Fc-CNP ELISA, 5 g/mL of Human CNP monoclonal antibody was coated on the plates. Vector TQQ (Fc1-CNP36) standard proteins range lies between 25-0.4 ng/ml. The bound Fc-CNP proteins were detected with Biotin-conjugated Goat anti Human IgG polyclonal antibody (1:10,000 dilution) and Streptavidin-HRP conjugate (1:10000 dilution). For the Endostatin ELISA, 5 μg/mL of Mouse monoclonal [KT58] to Endostatin/COL 18A1 was coated on the plates. Human Endostatin Recombinant Protein, PeproTech®, was used as standards with the concentration range of 3-0.05 ng/mL. The bound endostatin fusion protein was detected with Goat-anti-Human IgG polyclonal antibody (Biotin) (1:10,000 dilution) and Streptavidin-HRP (1:10,000 dilution).

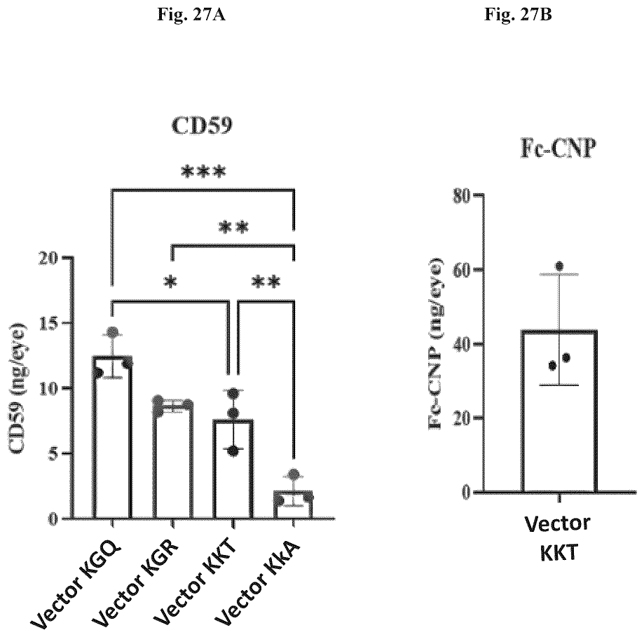

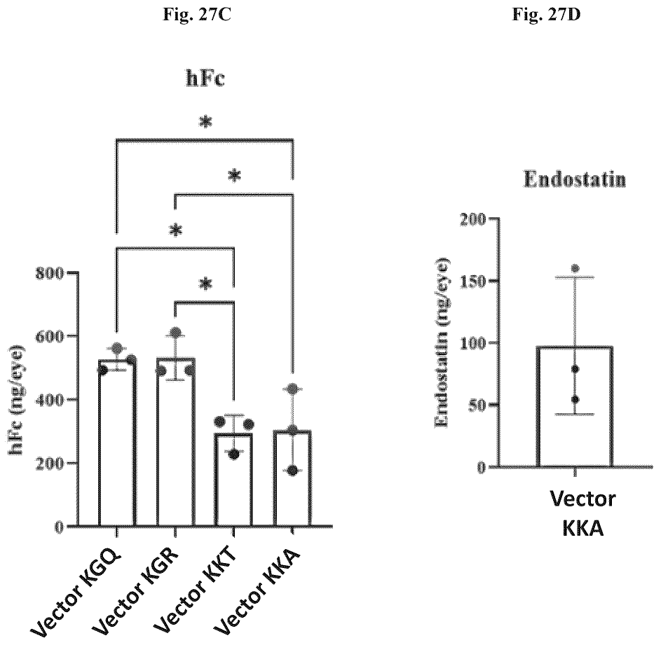

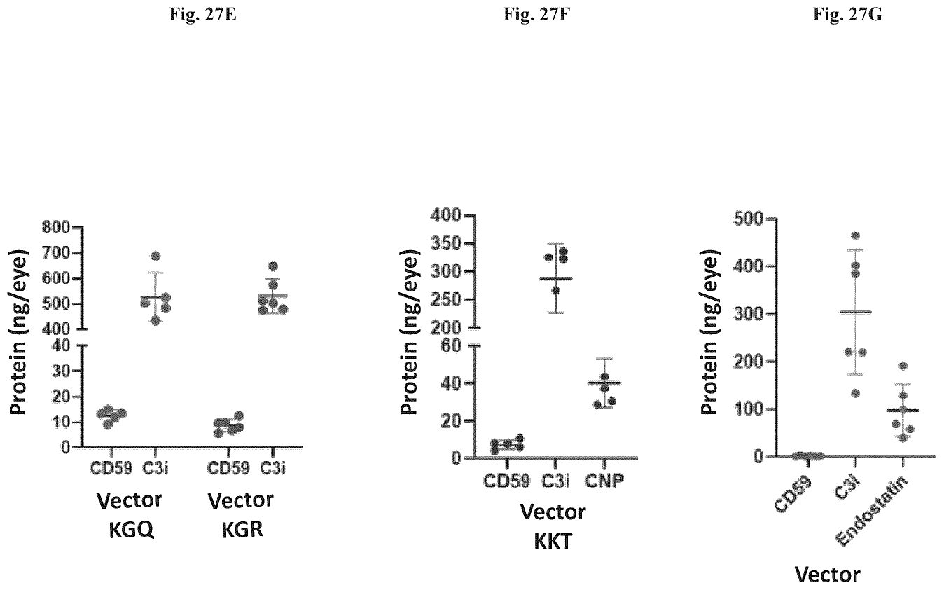

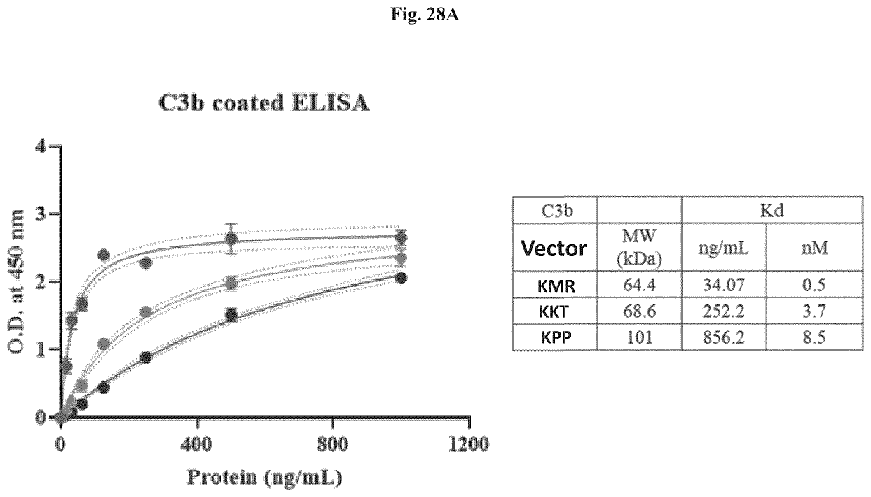

Eye homogenates were tested for all four gene products. Table 35 shows the data of eye homogenates in ng/mL. The data was statistically analyzed with GraphPad Prism software using One-Way ANOVA with Tukey's multiple comparison. The concentrations of sCD59 and C3i were significantly higher in Vector KGQ compared to Vector KKT (FIG. 27A) and Vector KKA (FIG. 27C). Like in vitro expression, CBA promoter also showed higher expression of sCD59 when compared to CAG promoter. Although there was a slight, non-significant decrease in sCD59 levels in Vector KGR compared to Vector KGQ, Vector KGR still showed higher levels than Vector KKT and Vector KKA. In addition, Vector KKT and Vector KKA had detection with the CNP36 (FIG. 27B) and endostatin (FIG. 27D) expression. To depict the expression landscape of each AAV, proteins of interest for each individual construct, Vector KGQ and Vector KGR (FIG. 27E), Vector KKT (FIG. 27F), and Vector KKA (FIG. 27G) were shown together.

TABLE 35
Protein concentrations of sCD59, C3i, Fc-CNP36 and
Endostatin in Eye Homogenate (ng/mL)

| | | Concentration (ng/mL) | | | |
|---|---|---|---|---|---|
| Group/Test Article | Eye | CD59 | C3i | CNP | Endostatin |
| 1 | 251 OS | 39.9 | 2066.4 | N.A. | N.A. |
| Vector KGQ | 251 OD | 29.2 | 1399.4 | N.A. | N.A. |
| | 252 OS | 47.1 | 1748.6 | N.A. | N.A. |
| | 252 OD | 51.9 | 1678.2 | N.A. | N.A. |
| | 253 OS | 37.5 | 1656.3 | N.A. | N.A. |
| 2 | 254 OS | 38.0 | 1764.1 | N.A. | N.A. |
| Vector KGR | 254 OD | 17.7 | 1995.3 | N.A. | N.A. |
| | 255 OS | 24.8 | 1607.3 | N.A. | N.A. |
| | 255 OD | 33.1 | 1640.5 | N.A. | N.A. |
| | 256 OS | 29.9 | 1478.2 | N.A. | N.A. |
| | 256 OD | 20.2 | 1505.3 | N.A. | N.A. |
| 3 | 257 OS | 25.3 | 1011.1 | 183.2 | N.A. |
| Vector KKT | 258 OS | 12.9 | 591.4 | 92.6 | N.A. |
| | 258 OD | 20.2 | 873.8 | 112.6 | N.A. |
| | 259 OS | 25.9 | 1014.1 | 86.7 | N.A. |
| | 259 OD | 48.9 | 1515.4 | 131.3 | N.A. |
| 4 | 260 OS | 8.6 | 1226.4 | N.A. | 392.6 |
| Vector KKA | 260 OD | 14.1 | 1655.1 | N.A. | 681.0 |
| | 261 OS | 6.0 | 737.9 | N.A. | 196.3 |
| | 261 OD | 5.0 | 1276.2 | N.A. | 328.8 |
| | 262 OS | 4.2 | 716.0 | N.A. | 224.9 |
| | 262 OD | 5.1 | 470.5 | N.A. | 139.9 |

N.A. = Not Applicable

Blood was collected at tine time of sacrifice on day 28 and stored. Table 36 shows the concentration of sCD59, C3i, Fc-CNP36 and Endostatin (in ng/mL) in the plasma along with the eye tissue concentration (in ng/eye). The sCD59 and endostatin concentrations were low in the plasma samples. CNP levels were also low except for one animal, #259. The same animal showed high C3i concentration as well. Overall, the concentration (for all gene products) in plasma was >10 fold lower than eye tissue.

TABLE 36
Protein concentration of sCD59, C3i, Fc-CNP36 and Endostatin
in the mouse plasma (ng/ml) and eye tissue (ng/eye)

| Animal | CD59 | C3i | CNP | Endostatin |
|---|---|---|---|---|
| # | (ng/eye) | (ng/eye) | (ng/eye) | (ng/eye) |
| | Whole eye | | | |
| 251 | 11.2 | 561.7 | N.D. | N.D. |
| 252 | 14.3 | 493.5 | N.D. | N.D. |
| 253 | 11.9 | 525.0 | N.D. | N.D. |
| 254 | 9.1 | 611.8 | N.D. | N.D. |
| 255 | 8.7 | 492.6 | N.D. | N.D. |
| 256 | 8.2 | 490.8 | N.D. | N.D. |
| 257 | 8.1 | 322.5 | 61.0 | N.D. |
| 258 | 5.2 | 228.5 | 34.2 | N.D. |
| 259 | 9.6 | 331.0 | 36.3 | N.D. |
| 260 | 3.4 | 433.7 | N.D. | 160.1 |
| 261 | 1.7 | 303.0 | N.D. | 79.0 |
| 262 | 1.4 | 177.0 | N.D. | 54.5 |
| | Plasma | | | |
| 251 | 0.0 | 23.2 | 0.2 | 0 |
| 252 | 0.0 | 20.9 | 0.0 | 0 |
| 253 | 0.0 | 29.4 | 0.1 | 0 |
| 254 | 14.1 | 27.9 | 0.0 | 0 |
| 255 | 0.0 | 19.4 | 0.1 | 0 |
| 256 | 0.0 | 22.3 | 0.0 | 0 |
| 257 | 0.0 | 48.1 | 0.7 | 0 |
| 258 | 4.2 | 63.6 | 0.6 | 0 |
| 259 | 48.0 | 168.6 | 19.4 | 0 |
| 260 | 0.0 | 4.7 | 0.0 | 0 |
| 261 | 0.0 | 6.1 | 0.1 | 0.01 |
| 262 | 0.0 | 11.4 | 0.0 | 0.01 |

N.D. = No Data

TABLE 37
DNA sequences of plasmids used in Example 10

| Vector | | | |
|---|---|---|---|
| name | Protein | Nucleic acid sequence (ORF for GOI) | SEQ ID NO |
| Vector | sCD59 | ATGGGCATCCAGGGCGGCAGCGTGCTGTTCGGCCTGCTG | 270 |
| KGQ, | | CTGGTGCTGGCCGTGTTCTGCCACAGCGGCCACAGCCTGC | |
| Vector | | AGTGCTACAACTGCCCCAACCCCACCGCCGACTGCAAGA | |
| KGR, | | CCGCCGTGAACTGCAGCAGCGACTTCGACGCCTGCCTGA | |
| Vector | | TCACCAAGGCCGGCCTGCAGGTGTACAACAAGTGCTGGA | |
| KKT, | | AGTTCGAGCACTGCAACTTCAACGACGTGACCACCAGGC | |
| Vector | | TGAGGGAGAACGAGCTGACCTACTACTGCTGCAAGAAGG | |
| KKA, | | ACCTGTGCAACTTCAACGAGCAGCTGGAGAACTAA | |
| Vector | | | |
| KMR, | | | |
| Vector | | | |
| KPT, | | | |
| Vector | | | |
| KPC, | | | |
| Vector | | | |
| KPP | | | |
| Vector | Fc4- | ATGGAGTTCGGCCTGAGCTGGCTGTTCCTGGTGGCCATCC | 271 |
| KGQ, | C3i(N15Q) | TTAAGGGCGTGCAGTGCGACAAGAGAGTGGAGAGCAAGT | |
| Vector | | ACGGCCCCCCCTGCCCCCCCTGCCCCGCCCCCGAGTTCCT | |
| KGR | | GGGCGGCCCCAGCGTGTTCCTGTTCCCCCCCAAGCCCAAG | |
| | | GACACCCTGATGATCAGCAGAACCCCCGAGGTGACCTGC | |
| | | GTGGTGGTGGACGTGAGCCAGGAGGACCCCGAGGTGCAG | |
| | | TTCAACTGGTACGTGGACGGCGTGGAGGTGCACAACGCC | |
| | | AAGACCAAGCCCAGAGAGGAGCAGTTCAACAGCACCTAC | |
| | | AGAGTGGTGAGCGTGCTGACCGTGCTGCACCAGGACTGG | |
| | | CTGAACGGCAAGGAGTACAAGTGCAAGGTGAGCAACAA | |
| | | GGGCCTGCCCAGCAGCATCGAGAAGACCATCAGCAAGGC | |
| | | CAAGGGCCAGCCCAGAGAGCCCCAGGTGTACACCCTGCC | |
| | | CCCCAGCCAGGAGGAGATGACCAAGAACCAGGTGAGCCT | |
| | | GACCTGCCTGGTGAAGGGCTTCTACCCCAGCGACATCGC | |
| | | CGTGGAGTGGGAGAGCAACGGCCAGCCCGAGAACAACT | |
| | | ACAAGACCACCCCCCCCGTGCTGGACAGCGACGGCAGCT | |
| | | TCTTCCTGTACAGCAGACTGACCGTGGACAAGAGCAGAT | |
| | | GGCAGGAGGGCAACGTGTTCAGCTGCAGCGTGATGCACG | |
| | | AGGCCCTGCACAACCACTACACCCAGAAGAGCCTGAGCC | |
| | | TGAGCCTGGGCAAGGGCGGAGGCGGAAGCGGCGGAGGC | |
| | | GGATCTGGCGGAGGCGGAAGCGGCGGAGGCGGCTCTGGC | |
| | | ATCTGCGTGTGGCAGGACTGGGGCGCCCACCGCTGCACC | |
| | | CAGTAA | |
| Vector | C3i(N15 | ATGGAGTTCGGCCTGAGCTGGCTGTTCCTGGTGGCCATCC | 272 |
| KKT | Q)-Fc4- | TTAAGGGCGTGCAGTGCGACAAGGGCATCTGCGTGTGGC | |
| | CNP36 | AGGACTGGGGCGCCCACCGCTGCACCCAGGGCGGAGGCG | |
| | | GAAGCGGCGGAGGCGGATCTGGCGGAGGCGGAAGCGAC | |
| | | AAGAGAGTGGAGAGCAAGTACGGCCCCCCCTGCCCCCCC | |
| | | TGCCCCGCCCCCGAGTTCCTGGGCGGCCCCAGCGTGTTCC | |
| | | TGTTCCCCCCCAAGCCCAAGGACACCCTGATGATCAGCA | |
| | | GAACCCCCGAGGTGACCTGCGTGGTGGTGGACGTGAGCC | |
| | | AGGAGGACCCCGAGGTGCAGTTCAACTGGTACGTGGACG | |
| | | GCGTGGAGGTGCACAACGCCAAGACCAAGCCCAGAGAG | |
| | | GAGCAGTTCAACAGCACCTACAGAGTGGTGAGCGTGCTG | |
| | | ACCGTGCTGCACCAGGACTGGCTGAACGGCAAGGAGTAC | |
| | | AAGTGCAAGGTGAGCAACAAGGGCCTGCCCAGCAGCATC | |
| | | GAGAAGACCATCAGCAAGGCCAAGGGCCAGCCCAGAGA | |
| | | GCCCCAGGTGTACACCCTGCCCCCCAGCCAGGAGGAGAT | |
| | | GACCAAGAACCAGGTGAGCCTGACCTGCCTGGTGAAGGG | |
| | | CTTCTACCCCAGCGACATCGCCGTGGAGTGGGAGAGCAA | |
| | | CGGCCAGCCCGAGAACAACTACAAGACCACCCCCCCCGT | |
| | | GCTGGACAGCGACGGCAGCTTCTTCCTGTACAGCAGACT | |
| | | GACCGTGGACAAGAGCAGATGGCAGGAGGGCAACGTGTT | |
| | | CAGCTGCAGCGTGATGCACGAGGCCCTGCACAACCACTA | |
| | | CACCCAGAAGAGCCTGAGCCTGAGCCTGGGCAAGGGCGG | |
| | | AGGCGGAAGCGGCGGAGGCGGATCTGGCGGAGGCGGAA | |
| | | GCGGCGGAGGCGGCTCTGAGCACCCCAACGCGCGCAAAT | |
| | | ACAAAGGAGCCAACAAGAAGGGCTTGTCCAAGGGCTGCT | |
| | | TCGGCCTCAAGCTGGACCGAATCGGCTCCATGAGCGGCC | |
| | | TGGGATGTTAG | |
| Vector | C3i(N15 | ATGGAGTTCGGCCTGAGCTGGCTGTTCCTGGTGGCCATCC | 273 |
| KKA | Q)-Fc4- | TTAAGGGCGTGCAGTGCGACAAGGGCATCTGCGTGTGGC | |
| | Endostatin | AGGACTGGGGCGCCCACCGCTGCACCCAGGGCGGAGGCG | |
| | | GAAGCGGCGGAGGCGGATCTGGCGGAGGCGGAAGCGAC | |
| | | AAGAGAGTGGAGAGCAAGTACGGCCCCCCCTGCCCCCCC | |
| | | TGCCCCGCCCCCGAGTTCCTGGGCGGCCCCAGCGTGTTCC | |
| | | TGTTCCCCCCCAAGCCCAAGGACACCCTGATGATCAGCA | |
| | | GAACCCCCGAGGTGACCTGCGTGGTGGTGGACGTGAGCC | |
| | | AGGAGGACCCCGAGGTGCAGTTCAACTGGTACGTGGACG | |
| | | GCGTGGAGGTGCACAACGCCAAGACCAAGCCCAGAGAG | |
| | | GAGCAGTTCAACAGCACCTACAGAGTGGTGAGCGTGCTG | |
| | | ACCGTGCTGCACCAGGACTGGCTGAACGGCAAGGAGTAC | |
| | | AAGTGCAAGGTGAGCAACAAGGGCCTGCCCAGCAGCATC | |
| | | GAGAAGACCATCAGCAAGGCCAAGGGCCAGCCCAGAGA | |
| | | GCCCCAGGTGTACACCCTGCCCCCCAGCCAGGAGGAGAT | |
| | | GACCAAGAACCAGGTGAGCCTGACCTGCCTGGTGAAGGG | |
| | | CTTCTACCCCAGCGACATCGCCGTGGAGTGGGAGAGCAA | |
| | | CGGCCAGCCCGAGAACAACTACAAGACCACCCCCCCCGT | |
| | | GCTGGACAGCGACGGCAGCTTCTTCCTGTACAGCAGACT | |
| | | GACCGTGGACAAGAGCAGATGGCAGGAGGGCAACGTGTT | |
| | | CAGCTGCAGCGTGATGCACGAGGCCCTGCACAACCACTA | |
| | | CACCCAGAAGAGCCTGAGCCTGAGCCTGGGCAAGGGCGG | |
| | | AGGCGGAAGCGGCGGAGGCGGATCTGGCGGAGGCGGAA | |
| | | GCGGCGGAGGCGGCTCTCACAGCCACAGGGACTTCCAGC | |
| | | CTGTGCTGCACCTGGTGGCCCTGAACAGCCCTCTGAGCGG | |
| | | AGGAATGAGGGGAATCAGGGGAGCCGACTTCCAGTGCTT | |
| | | CCAGCAGGCCAGGGCCGTGGGACTGGCCGGAACCTTCAG | |
| | | GGCCTTCCTGAGCAGCAGGCTGCAGGACCTGTACAGCAT | |
| | | CGTGAGGAGGGCCGACAGGGCCGCCGTGCCCATCGTGAA | |
| | | CCTGAAGGACGAGCTGCTGTTCCCTAGCTGGGAGGCCCT | |
| | | GTTCAGCGGAAGCGAGGGACCCCTGAAGCCCGGAGCCAG | |
| | | GATCTTCAGCTTCGACGGAAAGGACGTGCTGAGGCACCC | |
| | | TACCTGGCCTCAGAAGAGCGTGTGGCACGGAAGCGACCC | |
| | | TAACGGAAGGAGGCTGACCGAGAGCTACTGCGAGACCTG | |
| | | GAGGACCGAGGCCCCTAGCGCCACCGGCCAGGCCAGCAG | |
| | | CCTGCTGGGAGGAAGGCTGCTGGGACAGAGCGCCGCCAG | |
| | | CTGCCACCACGCCTACATCGTGCTGTGCATCGAGAACAG | |
| | | CTTCATGACCGCCAGCAAGTGA | |

TABLE 38
Peptides and fusion protein sequences of Example 10

| Vector | | | |
|---|---|---|---|
| name | Protein | Nucleic acid sequence (ORF for GOI) | SEQ ID NO |
| Vector | sCD59 | MGIQGGSVLFGLLLVLAVFCHSGHSLQCYNCPNPTADCKT | 297 |
| KGQ, | | AVNCSSDFDACLITKAGLQVYNKCWKFEHCNFNDVTTRLR | |
| Vector | | ENELTYYCCKKDLCNFNEQLEN | |
| KGR, | | | |
| Vector | | | |
| KKT, | | | |
| Vector | | | |
| KKA, | | | |
| Vector | | | |
| KMR, | | | |
| Vector | | | |
| KPT, | | | |
| Vector | | | |
| KPC, | | | |
| Vector | | | |
| KPP | | | |
| Vector | Fc4-C3i, | MEFGLSWLFLVAILKGVQCDKRVESKYGPPCPPCPAPEFLG | 298 |
| KGQ, | | GPSVFLFPPKPKDTLMISRTPEVTCVVVDVSQEDPEVQFNW | |
| Vector | | YVDGVEVHNAKTKPREEQFNSTYRVVSVLTVLHQDWLNG | |
| KGR | | KEYKCKVSNKGLPSSIEKTISKAKGQPREPQVYTLPPSQEE | |
| | | MTKNQVSLTCLVKGFYPSDIAVEWESNGQPENNYKTTPPV | |
| | | LDSDGSFFLYSRLTVDKSRWQEGNVFSCSVMHEALHNHYT | |
| | | QKSLSLSLGKGGGGSGGGGSGGGGSGGGGSGICVWQDWG | |
| | | AHRCTQ | |
| Vector | C3i | MEFGLSWLFLVAILKGVQCDKGICVWQDWGAHRCTQGGG | 299 |
| KKT | (N15Q)- | GSGGGGSGGGGSDKRVESKYGPPCPPCPAPEFLGGPSVFLF | |
| | Fc4- | PPKPKDTLMISRTPEVTCVVVDVSQEDPEVQFNWYVDGVE | |
| | CNP36 | VHNAKTKPREEQFNSTYRVVSVLTVLHQDWLNGKEYKCK | |
| | | VSNKGLPSSIEKTISKAKGQPREPQVYTLPPSQEEMTKNQV | |
| | | SLTCLVKGFYPSDIAVEWESNGQPENNYKTTPPVLDSDGSF | |
| | | FLYSRLTVDKSRWQEGNVFSCSVMHEALHNHYTQKSLSLS | |
| | | LGKGGGGSGGGGSGGGGSGGGGSEHPNARKYKGANKKG | |
| | | LSKGCFGLKLDRIGSMSGLGC | |
| Vector | C3i(N15 | MEFGLSWLFLVAILKGVQCDKGICVWQDWGAHRCTQGGG | 300 |
| KKA | Q)-Fc4- | GSGGGGSGGGGSDKRVESKYGPPCPPCPAPEFLGGPSVFLF | |
| | Endostatin | PPKPKDTLMISRTPEVTCVVVDVSQEDPEVQFNWYVDGVE | |
| | | VHNAKTKPREEQFNSTYRVVSVLTVLHQDWLNGKEYKCK | |
| | | VSNKGLPSSIEKTISKAKGQPREPQVYTLPPSQEEMTKNQV | |
| | | SLTCLVKGFYPSDIAVEWESNGQPENNYKTTPPVLDSDGSF | |
| | | FLYSRLTVDKSRWQEGNVFSCSVMHEALHNHYTQKSLSLS | |
| | | LGKGGGGSGGGGSGGGGSGGGGSHSHRDFQPVLHLVALN | |
| | | SPLSGGMRGIRGADFQCFQQARAVGLAGTFRAFLSSRLQD | |
| | | LYSIVRRADRAAVPIVNLKDELLFPSWEALFSGSEGPLKPG | |
| | | ARIFSFDGKDVLRHPTWPQKSVWHGSDPNGRRLTESYCET | |
| | | WRTEAPSATGQASSLLGGRLLGQSAASCHHAYIVLCIENSF | |
| | | MTASK | |

### Example 11. Ligand Binding and/or Function Assay of C3i Fusion Proteins

### Ligand Binding Assay of C3i Fusion Proteins

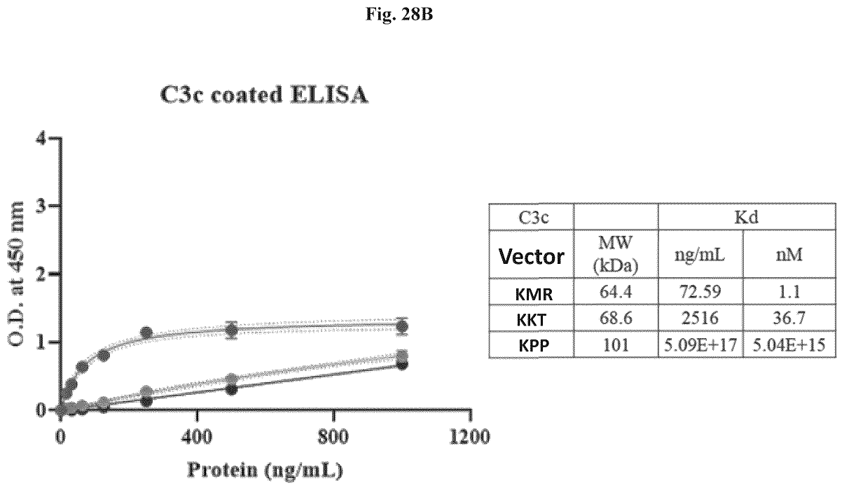

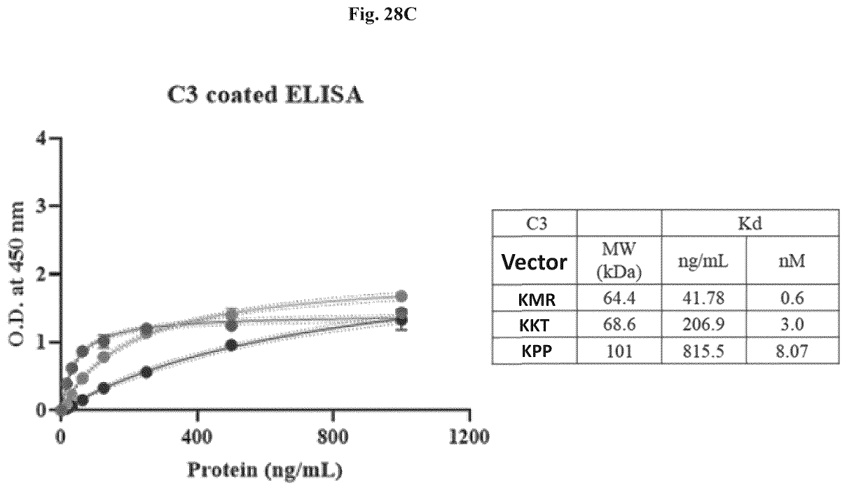

To test the binding affinity of C3i fusion proteins against C3i ligands by ELISA and develop the potency assay for clinical trial, C3b (FIG. 28A), C3c (FIG. 28B), or C3 (FIG. 28C) protein was coated on the plates overnight. After wash for three times, the C3i fusion proteins, Vector KMR, Vector KKT, and Vector KPP, were added onto the plates. The bound proteins were detected with HRP-conjugated anti-human Fc antibody. The data was fit using hyperbolic equation on GraphPad Prism software and Kd value was determined. Vector KMR shows the higher binding affinity than Vector KKT, followed by Vector KPP against all these proteins, C3b, C3c and C3. However, compared to C3 and C3b all C3i fusion proteins have the low binding affinity against C3c, and similar between C3b and C3.

### Ligand Binding and Function Assay of CNP36 Portion in C3i-Fc4-CNP36 (Vector KKT)

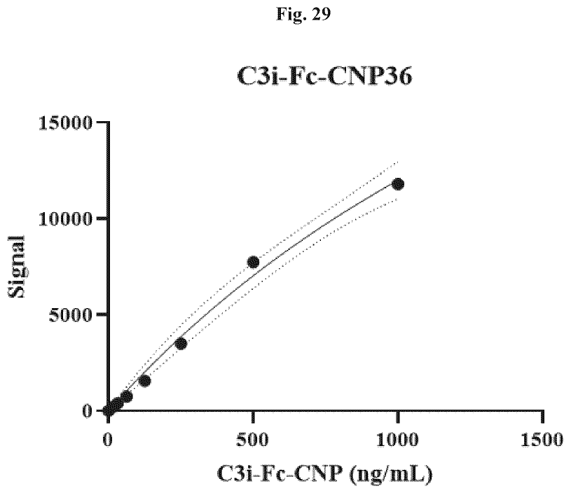

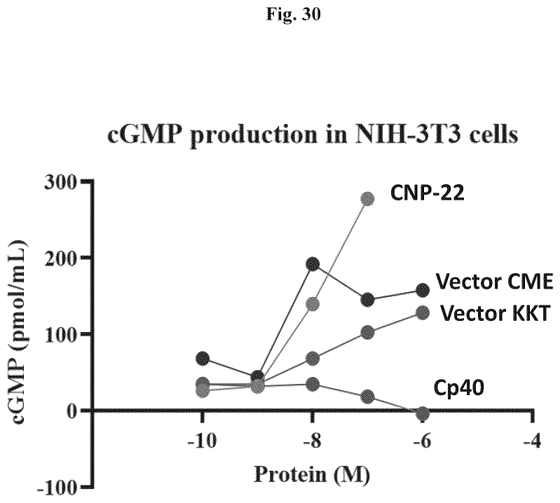

Although C3i portion in C3i-Fc4-CNP36 showed biological function, CNP36 function is to be demonstrated. Ligand binding assay was performed for Vector KKT (C3i-Fc4-CNP36). Initially, 0.1 μg/mL NPR-b protein was coated on the plates. Then the fusions proteins, Vector KKT protein was added onto the plates. The bound proteins were detected with Biotin-labelled anti-human Fc antibodies and Streptavidin-Sulfo tag. The electro-chemiluminescence signal was detected using a MSD plate reader. C3i-Fc-SNP36 showed binding to NPR-b in a dose-dependent manner (FIG. 29).

On the other hand, cGMP production was quantified in NIH-3T3 cells triggered by C3i-Fc4-CNP36 (Vector KKT), Fc4-CNP36 (Vector CME), CNP-22 peptide and CP40. In FIG. 30 was shown that CNP22 and CNP-containing proteins, Vector CME and Vector KKT, could stimulate cGMP production in NIH-3T3 cells, but not the CP40 peptide. It suggests that, as Fc4-CNP36, the expressed C3i-Fc4-CNP36 had the same function.

### Ligand Binding Assay of Endostatin Portion in C3i-Fc4-Endostatin (Vector KPP)

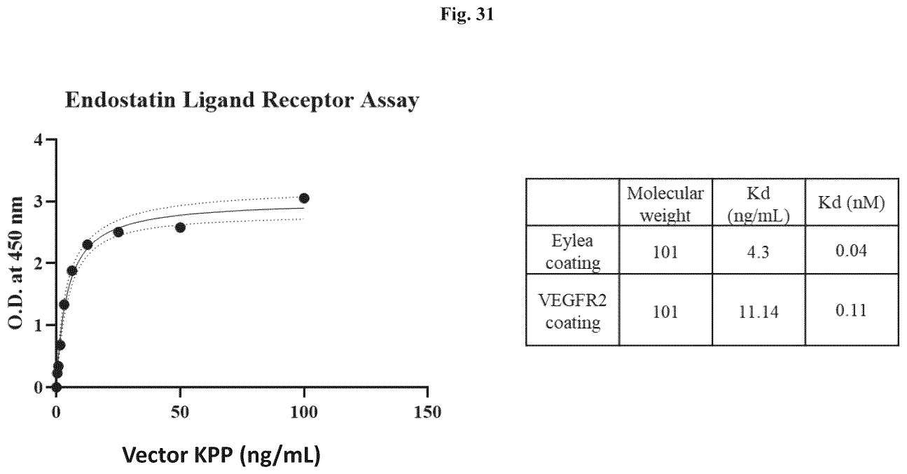

In the same, C3i function in C3i-Fc4-endostatin (Vector KPP) had been shown above, but endostatin function is to be tested. Here the Eylea or VEGFR2 proteins were coated on the plates. After wash for three times, Vector KPP proteins were added onto the plates and the bound proteins were detected with HRP-conjugated anti-human endostatin antibodies. The data was plotted by GraphPad Prism software and Kd value was estimated accordingly. The result shows commercial drug (Eylea) or pure VEGFR2 proteins have very high binding affinity against Endostatin ligands (FIG. 31).

### Example 12. Optimization of vsCD59 Protein Engineering

Designing and Molecular Cloning of Variant Soluble CD59 (vsCD59) Genes

To improve the sCD59 biological activities, a series of mutations were prepared based on the plasmid Vector GEM. First, the 6×His tag-encoding sequences were cloned to the 3′ end of sCD59 gene for the convenient protein purification, generating the plasmid Vector KRE. The ORF sequences of GOI and their encoded protein sequences are listed in Table 49 and Table 50, respectively. After the sCD59-6×His protein (Vector KRE) expression was confirmed, single or multiple mutations were introduced to sCD59 gene on the plasmid Vector KRE and the resulting plasmids were generated (Table 46).

TABLE 46
sCD59 mutations introduced on the plasmid Vector GEM

| Plasmids | Signal peptide | Mutations | Add |
|---|---|---|---|
| Vector GEM | wild-type | wild-type | No |
| Vector KRC | Vh(DK) | wild-type | No |
| Vector KRE | wild-type | wild-type | 6 × His |
| Vector KRG | wild-type | N18Q | 6 × His |
| Vector KRK | wild-type | Q34E | 6 × His |
| Vector KRM | wild-type | K38R | 6 × His |
| Vector KRP | wild-type | Q34E, K38R | 6 × His |
| Vector MTQ | wild-type | N18Q, Q34E | 6 × His |
| Vector KRQ | wild-type | N18Q, K38R | 6 × His |
| Vector KRR | wild-type | N18Q, Q34E, | 6 × His |
| | | K38R | |
| Vector MTT | wild-type | L33E | 6 × His |
| Vector MTA | wild-type | S20G | 6 × His |
| Vector MTC | wild-type | T51G | 6 × His |
| Vector MTE | wild-type | S20G, T51G | 6 × His |

Transient Expression of vsCD59 Genes in Mammalian Cell Culture System

While the mutations were cloned and the plasmid sequences were verified, a large amount of plasmids were prepared by maxiprep. These plasmids were transfected into Expi293 cells following the procedure shown in Example 3. Three days after transfection, media were harvested for vsCD59 protein detection by ELISA. The vsCD59 expression was displayed in the Table 47. The mutants containing the mutation Q34E were not expressed, neither was the Vector MTT (L33E). Other expressed vsCD59 proteins were directly used for testing their binding with the ligand C9 protein by MSD.

TABLE 47
Transient expression of vsCD59 in the Expi293 cells

| Plasmids | Mutations | Protein expression (μg/mL) | |
|---|---|---|---|
| Vector GEM | wild-type | 3.44 | * |
| Vector KRC | wild-type | 0.1 | * |
| Vector KRE | wild-type with 6 × His | 3.15 | |
| Vector KRG | N18Q | 1.22 | |
| Vector KRK | Q34E | 0 | |
| Vector KRM | K38R | 5.58 | |
| Vector KRP | Q34E, K38R | 0 | |
| Vector MTQ | N18Q, Q34E | 0 | |
| Vector KRQ | N18Q, K38R | N/A | |
| Vector KRR | N18Q, Q34E, K38R | 0 | |
| Vector MTT | L33E | 0 | |
| Vector MTA | S20G | 5.05 | |
| Vector MTC | T51G | 2.88 | |
| Vector MTE | S20G, T51G | 1.85 | |

* Vector GEM and Vector KRC was expressed in HEK293LTV cells on the 6-well plates.

Determination of Binding Affinity of vsCD59 Against C9 Protein

The binding assay for full-length CD59, soluble(s) CD59, and variant sCD59 to C9 was conducted by immobilizing 5 μg/mL of C9 onto an MSD 96-well Sector plate. The plate was subsequently blocked for 1 hour at room temperature with gentle shaking. CD59 variants were titrated starting from 1 μg/mL down to 0.015 μg/mL. A mouse monoclonal anti-CD59 antibody conjugated to biotin was then added at a 1:1000 dilution, followed by the addition of Streptavidin-Sulfo tag at a 1:5000 dilution. After the addition of the read buffer, the electrochemiluminescence signal was detected using an MSD plate reader. The resulting binding curves were analyzed using a hyperbolic equation in GraphPad Prism software, and the dissociation constant (Kd) was determined.

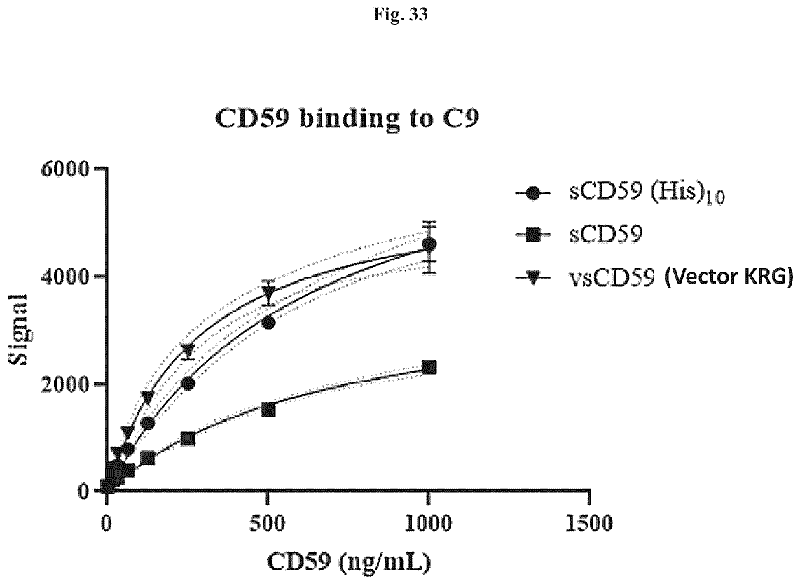

When the different amount of wild-type or variant sCD59 was used for the binding assay, they were plotted against the electrochemiluminescence signal (FIG. 33) for determining their binding affinity against C9 proteins. The Kd value for the vcCD59 proteins (Vector KRE, Vector KRG, Vector KRM, Vector MTA, Vector MTC, and Vector MTE) and the commercial and in-house purified sCD59 are shown in Table 48. The wild-type sCD59-6×His had the similar Kd value to the commercial or in-house purified sCD59. Among the vsCD59 proteins, only Vector KRG (N18Q) showed roughly 2 times lower Kd value than the wild-type sCD59.

TABLE 48
Kd values for CD59 proteins

| | | | vsCD59 | vsCD59 | vsCD59 | vsCD59 | vsCD59 | vsCD59 |
|---|---|---|---|---|---|---|---|---|
| | sCD59 | | Vector | Vector | Vector | Vector | Vector | Vector |
| Proteins | (His)₁₀ | sCD59 | KRE | KRG | KRM | MTA | MTC | MTE |
| Mutation | Wild- | Wild- | Wild- | N18Q | K38R | S20G | T51G | S20G, |
| | type | type | type | | | | | T51G |
| Kd (nM) | 33.1 | 38.6 | 34.3 | 16 | 39 | 46.1 | 23.7 | 28.9 |

TABLE 49
DNA sequences of plasmids of Example 12

| Vector | | | |
|---|---|---|---|
| name | Protein | Nucleic acid sequence (ORF for GOI) | SEQ ID NO |
| Vector | (DK) | ATGGAGTTCGGCCTGAGCTGGCTGTTCCTGGTGGCCATC | 304 |
| KRC | vsCD59 | CTTAAGGGCGTGCAGTGCGACAAGCTGCAGTGCTACAAC | |
| | | TGCCCCAACCCCACCGCCGACTGCAAGACCGCCGTGAAC | |
| | | TGCAGCAGCGACTTCGACGCCTGCCTGATCACCAAGGCC | |
| | | GGCCTGCAGGTGTACAACAAGTGCTGGAAGTTCGAGCA | |
| | | CTGCAACTTCAACGACGTGACCACCAGGCTGAGGGAGA | |
| | | ACGAGCTGACCTACTACTGCTGCAAGAAGGACCTGTGCA | |
| | | ACTTCAACGAGCAGCTGGAGAACTAA | |
| Vector | vsCD59 | ATGGGCATCCAGGGCGGCAGCGTGCTGTTCGGCCTGCTG | 305 |
| KRE | | CTGGTGCTGGCCGTGTTCTGCCACAGCGGCCACAGCCTG | |
| | | CAGTGCTACAACTGCCCCAACCCCACCGCCGACTGCAAG | |
| | | ACCGCCGTGAACTGCAGCAGCGACTTCGACGCCTGCCTG | |
| | | ATCACCAAGGCCGGCCTGCAGGTGTACAACAAGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | vsCD59 | ATGGGCATCCAGGGCGGCAGCGTGCTGTTCGGCCTGCTG | 306 |
| KRG | N18Q | CTGGTGCTGGCCGTGTTCTGCCACAGCGGCCACAGCCTG | |
| | | CAGTGCTACAACTGCCCCAACCCCACCGCCGACTGCAAG | |
| | | ACCGCCGTGCAATGCAGCAGCGACTTCGACGCCTGCCTG | |
| | | ATCACCAAGGCCGGCCTGCAGGTGTACAACAAGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | vsCD59 | ATGGGCATCCAGGGCGGCAGCGTGCTGTTCGGCCTGCTG | 307 |
| KRK | Q34E | CTGGTGCTGGCCGTGTTCTGCCACAGCGGCCACAGCCTG | |
| | | CAGTGCTACAACTGCCCCAACCCCACCGCCGACTGCAAG | |
| | | ACCGCCGTGAACTGCAGCAGCGACTTCGACGCCTGCCTG | |
| | | ATCACCAAGGCCGGCCTGGAGGTGTACAACAAGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | vsCD59 | ATGGGCATCCAGGGCGGCAGCGTGCTGTTCGGCCTGCTG | 308 |
| KRM | K38R | CTGGTGCTGGCCGTGTTCTGCCACAGCGGCCACAGCCTG | |
| | | CAGTGCTACAACTGCCCCAACCCCACCGCCGACTGCAAG | |
| | | ACCGCCGTGAACTGCAGCAGCGACTTCGACGCCTGCCTG | |
| | | ATCACCAAGGCCGGCCTGCAGGTGTACAACCGGTGCTGG | |
| | | AAGTTCGAGCACTGCAACTTCAACGACGTGACCACCAGG | |
| | | CTGAGGGAGAACGAGCTGACCTACTACTGCTGCAAGAA | |
| | | GGACCTGTGCAACTTCAACGAGCAGCTGGAGAACCACC | |
| | | ATCACCATCACCATTAA | |
| Vector | vsCD59 | ATGGGCATCCAGGGCGGCAGCGTGCTGTTCGGCCTGCTG | 309 |
| KRP | Q34E, | CTGGTGCTGGCCGTGTTCTGCCACAGCGGCCACAGCCTG | |
| | K38R | CAGTGCTACAACTGCCCCAACCCCACCGCCGACTGCAAG | |
| | | ACCGCCGTGAACTGCAGCAGCGACTTCGACGCCTGCCTG | |
| | | ATCACCAAGGCCGGCCTGGAGGTGTACAACCGGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | vsCD59 | ATGGGCATCCAGGGCGGCAGCGTGCTGTTCGGCCTGCTG | 310 |
| KRQ | N18Q, | CTGGTGCTGGCCGTGTTCTGCCACAGCGGCCACAGCCTG | |
| | K38R | CAGTGCTACAACTGCCCCAACCCCACCGCCGACTGCAAG | |
| | | ACCGCCGTGCAATGCAGCAGCGACTTCGACGCCTGCCTG | |
| | | ATCACCAAGGCCGGCCTGCAGGTGTACAACCGGTGCTGG | |
| | | AAGTTCGAGCACTGCAACTTCAACGACGTGACCACCAGG | |
| | | CTGAGGGAGAACGAGCTGACCTACTACTGCTGCAAGAA | |
| | | GGACCTGTGCAACTTCAACGAGCAGCTGGAGAACCACC | |
| | | ATCACCATCACCATTAA | |
| Vector | vsCD59 | ATGGGCATCCAGGGCGGCAGCGTGCTGTTCGGCCTGCTG | 311 |
| KRR | N18Q, | CTGGTGCTGGCCGTGTTCTGCCACAGCGGCCACAGCCTG | |
| | Q34E, | CAGTGCTACAACTGCCCCAACCCCACCGCCGACTGCAAG | |
| | K38R | ACCGCCGTGCAATGCAGCAGCGACTTCGACGCCTGCCTG | |
| | | ATCACCAAGGCCGGCCTGGAGGTGTACAACCGGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | SCD59 | ATGGGCATCCAGGGCGGCAGCGTGCTGTTCGGCCTGCTG | 320 |
| MTT | L33E- | CTGGTGCTGGCCGTGTTCTGCCACAGCGGCCACAGCCTG | |
| | 6 × His | CAGTGCTACAACTGCCCCAACCCCACCGCCGACTGCAAG | |
| | | ACCGCCGTGAACTGCAGCAGCGACTTCGACGCCTGCCTG | |
| | | ATCACCAAGGCCGGCGAGCAGGTGTACAACAAGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | sCD59 | ATGGGCATCCAGGGCGGCAGCGTGCTGTTCGGCCTGCTG | 321 |
| MTA | S20G- | CTGGTGCTGGCCGTGTTCTGCCACAGCGGCCACAGCCTG | |
| | 6 × His | CAGTGCTACAACTGCCCCAACCCCACCGCCGACTGCAAG | |
| | | ACCGCCGTGAACTGCGGCAGCGACTTCGACGCCTGCCTG | |
| | | ATCACCAAGGCCGGCCTGCAGGTGTACAACAAGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | sCD59 | ATGGGCATCCAGGGCGGCAGCGTGCTGTTCGGCCTGCTG | 322 |
| MTC | T51G- | CTGGTGCTGGCCGTGTTCTGCCACAGCGGCCACAGCCTG | |
| | 6 × His | CAGTGCTACAACTGCCCCAACCCCACCGCCGACTGCAAG | |
| | | ACCGCCGTGAACTGCAGCAGCGACTTCGACGCCTGCCTG | |
| | | ATCACCAAGGCCGGCCTGCAGGTGTACAACAAGTGCTG | |
| | | GAAGTTCGAGCACTGCAACTTCAACGACGTGGGCACCA | |
| | | GGCTGAGGGAGAACGAGCTGACCTACTACTGCTGCAAG | |
| | | AAGGACCTGTGCAACTTCAACGAGCAGCTGGAGAACCA | |
| | | CCATCACCATCACCATTAAGTTAA | |
| Vector | sCD59 | ATGGGCATCCAGGGCGGCAGCGTGCTGTTCGGCCTGCTG | |
| MTE | S20G, | CTGGTGCTGGCCGTGTTCTGCCACAGCGGCCACAGCCTG | |
| | T51G- | CAGTGCTACAACTGCCCCAACCCCACCGCCGACTGCAAG | |
| | 6 × His | ACCGCCGTGAACTGCGGCAGCGACTTCGACGCCTGCCTG | |
| | | ATCACCAAGGCCGGCCTGCAGGTGTACAACAAGTGCTG | |
| | | GAAGTTCGAGCACTGCAACTTCAACGACGTGGGCACCA | |
| | | GGCTGAGGGAGAACGAGCTGACCTACTACTGCTGCAAG | |
| | | AAGGACCTGTGCAACTTCAACGAGCAGCTGGAGAACCA | |
| | | CCATCACCATCACCATTAA | 323 |
| Vector | sCD59 | ATGGGCATCCAGGGCGGCAGCGTGCTGTTCGGCCTGCTG | |
| MTQ | N18Q, | CTGGTGCTGGCCGTGTTCTGCCACAGCGGCCACAGCCTG | |
| | Q34E- | CAGTGCTACAACTGCCCCAACCCCACCGCCGACTGCAAG | |
| | 6 × His | ACCGCCGTGCAATGCAGCAGCGACTTCGACGCCTGCCTG | |
| | | ATCACCAAGGCCGGCCTGGAGGTGTACAACAAGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | 324 |
| | | CATCACCATCACCATTAA | |

TABLE 50
Peptides and fusion protein sequences of Example 12

| Vector | | | |
|---|---|---|---|
| name | Protein | Nucleic acid sequence (ORF for GOI) | SEQ ID NO |
| Vector | (DK) | MEFGLSWLFLVAILKGVQCDKLQCYNCPNPTADCKTAVN | 312 |
| KRC | vsCD59 | CSSDFDACLITKAGLQVYNKCWKFEHCNFNDVTTRLRENE | |
| | | LTYYCCKKDLCNFNEQLEN | |
| Vector | vsCD59 | MGIQGGSVLFGLLLVLAVFCHSGHSLQCYNCPNPTADCKT | 313 |
| KRE | | AVNCSSDFDACLITKAGLQVYNKCWKFEHCNFNDVTTRLR | |
| | | ENELTYYCCKKDLCNFNEQLENHHHHHH | |
| Vector | vsCD59 | MGIQGGSVLFGLLLVLAVFCHSGHSLQCYNCPNPTADCKT | 314 |
| KRG | N18Q | AVQCSSDFDACLITKAGLQVYNKCWKFEHCNFNDVTTRLR | |
| | | ENELTYYCCKKDLCNFNEQLENHHHHHH | |
| Vector | vsCD59 | MGIQGGSVLFGLLLVLAVFCHSGHSLQCYNCPNPTADCKT | 315 |
| KRK | Q34E | AVNCSSDFDACLITKAGLEVYNKCWKFEHCNFNDVTTRLR | |
| | | ENELTYYCCKKDLCNFNEQLENHHHHHH | |
| Vector | vsCD59 | MGIQGGSVLFGLLLVLAVFCHSGHSLQCYNCPNPTADCKT | 316 |
| KRM | K38R | AVNCSSDFDACLITKAGLQVYNRCWKFEHCNFNDVTTRLR | |
| | | ENELTYYCCKKDLCNFNEQLENHHHHHH | |
| Vector | vsCD59 | MGIQGGSVLFGLLLVLAVFCHSGHSLQCYNCPNPTADCKT | 317 |
| KRP | Q34E, | AVNCSSDFDACLITKAGLEVYNRCWKFEHCNFNDVTTRLR | |
| | K38R | ENELTYYCCKKDLCNFNEQLENHHHHHH | |
| Vector | vsCD59 | MGIQGGSVLFGLLLVLAVFCHSGHSLQCYNCPNPTADCKT | 318 |
| KRQ | N18Q, | AVQCSSDFDACLITKAGLQVYNRCWKFEHCNFNDVTTRLR | |
| | K38R | ENELTYYCCKKDLCNFNEQLENHHHHHH | |
| Vector | vsCD59 | MGIQGGSVLFGLLLVLAVFCHSGHSLQCYNCPNPTADCKT | 319 |
| KRR | N18Q, | AVQCSSDFDACLITKAGLEVYNRCWKFEHCNFNDVTTRLR | |
| | Q34E, | ENELTYYCCKKDLCNFNEQLENHHHHHH | |
| | K38R | | |
| Vector | sCD59 | MGIQGGSVLFGLLLVLAVFCHSGHSLQCYNCPNPTADCKT | 325 |
| MTT | L33E- | AVNCSSDFDACLITKAGEQVYNKCWKFEHCNFNDVTTRLR | |
| | 6×His | ENELTYYCCKKDLCNFNEQLENHHHHHH | |
| Vector | sCD59 | MGIQGGSVLFGLLLVLAVFCHSGHSLQCYNCPNPTADCKT | 326 |
| MTA | S20G- | AVNCGSDFDACLITKAGLQVYNKCWKFEHCNFNDVTTRL | |
| | 6 × His | RENELTYYCCKKDLCNFNEQLENHHHHHH | |
| Vector | sCD59 | MGIQGGSVLFGLLLVLAVFCHSGHSLQCYNCPNPTADCKT | 327 |
| MTC | T51G- | AVNCSSDFDACLITKAGLQVYNKCWKFEHCNFNDVGTRL | |
| | 6 × His | RENELTYYCCKKDLCNFNEQLENHHHHHH | |
| Vector | SCD59 | MGIQGGSVLFGLLLVLAVFCHSGHSLQCYNCPNPTADCKT | 328 |
| MTE | S20G, | AVNCGSDFDACLITKAGLQVYNKCWKFEHCNFNDVGTRL | |
| | T51G- | RENELTYYCCKKDLCNFNEQLENHHHHHH | |
| | 6 × His | | |
| Vector | sCD59 | MGIQGGSVLFGLLLVLAVFCHSGHSLQCYNCPNPTADCKT | 329 |
| MTQ | N18Q, | AVQCSSDFDACLITKAGLEVYNKCWKFEHCNFNDVTTRLR | |
| | Q34E- | ENELTYYCCKKDLCNFNEQLENHHHHHH | |
| | 6×His | | |

### Example 13. Cloning and Expression of CD59 Genes

Designing and molecular cloning of variant soluble CD59 (vsCD59) genes vsCD59 with a signal peptide-encoding sequences at the 5′-end are constructed into a self-complementary AAV vector. After a synthetic DNA fragment is successfully cloned into a vector, all plasmid DNA is identified by restriction digestion, and the sequences from all these plasmids are verified by compete sequencing. The ORF sequences of GOI and their encoded protein sequences are listed in Table 39 and Table 40, respectively. FIG. 32 illustrates additional exemplary vectors for expressing the angiogenesis inhibitors described herein.

Transient Expression of vsCD59 Genes in Mammalian Cell Culture System

Human HEK293LTV cells are used for vsCD59 gene expression and are cultured in DMEM medium (Thermo Fisher Scientific) with 10% FBS (ATCC, Manassas, VA) in a CO₂ incubator at 37° C. For maintenance passage, cells are split by 1:10 twice a week. For transfection, cells are seeded on 6-well plates (Corning, NY) at 0.6×10⁶ cells/well in 2 mL media overnight. One or two hours before transfection, the culture medium is replaced with DMEM medium with 2% FBS. For each well, 2 μg of plasmid DNA is diluted into 300 μL of 150 mM NaCl before 8 μL of PEImax (Polysciences, Germany) is added. After the mixture is incubated at room temperature for 20 minutes, it is added to the cells dropwise and incubated at 37° C. in the CO₂ incubator. Three days later, all medium from each well is harvested for further experiments, and 3 mL of fresh medium is added to the cells. In the same, the medium is collected in 3 days one more time.

After HEK293LTV cells are seeded onto 6-well plates 1 day prior to transient transfection, each transfection is performed with 2 μg/well DNA using PEImax. To evaluate the transfection efficiency, cells transfected with Vector EKQ are observed under fluorescence microscope. Cell culture supernatants at 3 days and 6 days post-transfection are collected and analyzed for protein expression.

vsCD59 proteins are detected by SDS-PAGE and Western blot analysis. Cell culture supernatant is collected at day 3 and 6 after plasmid DNA is transfected into the cells. A total volume of 26 μL of each supernatant is mixed with 10 μl of 4× loading buffer and 4 μl of 10× reducing buffer and loaded onto the NuPAGE™ 4-12% Bis-Tris gels (Thermo Fisher Scientific) for electrophoresis. After the proteins are run at 150 v for about 1 hour, one gel is stained by SimplyBlue™ SafeStain (Thermo Fisher Scientific), de-stained by water, and imaged using a digital camera. On the other hand, another gel is subsequently transferred onto PVDF membranes using Trans-Blot Turbo Transfer System (Bio-Rad, Hercules, CA, USA). Immediately after membranes are treated with casein blocker in PBS (Thermo Scientific, Waltham, MA, USA) for 1 hour at room temperature, they are probed with HRP-conjugated mouse-anti-human CD59 monoclonal antibody (sc-133170) (Santa Cruz Biotechnology, Dallas, TX). Proteins are detected using the Supersignal™ West Atto Ultimate sensitivity kit (Thermo Fisher Scientific) and images are captured by Gel Doc™ XR+ with Image Lab™ Software (Bio-Rad, Hercules, CA).

vsCD59 Protein Purification

A large-scale cell culture is prepared for protein purification. vsCD59 protein is purified using AKTA Explorer 100 in four steps. First, sample clarification is performed to remove cellular debris by centrifugation followed by 0.2 μm filtration (Nalgene, Rochester, NY). The clarified harvest is directly transferred into downstream purification. Second, gel filtration column chromatography is performed using Sephadex G-25 (Cytiva, Marlborugh, MA) to remove cell culture medium from the cell culture supernatant containing the CD59 protein. After the G-25 desalting column is equilibrated with Tris buffer (20 mM Tris pH7.3, 100 mM NaCl, 0.005% F-68), the cell culture supernatant containing the vsCD59 protein is loaded. The vsCD59 protein is collected when the UV A280 increases to 5 mAU and collection is stopped when the UV A280 reduced to 5 mAU. Third, a strong anion exchange column chromatography using HQ resin (ThermoFisher, Walthan, MA) is performed on the G-25 desalted pool. The G-25 desalted product is diluted with XQ column equilibration buffer (20 mM Tris pH7.3, 0.005% F-68) to a conductivity <3 mS/cm and loaded onto an equilibrated HQ column with equilibration buffer (20 mM Tris pH7.3, 10 mM NaCl, 0.005% F-68). After loading and washing with equilibration buffer, the column is eluted using a 10-500 mM NaCl gradient. The elution peak is collected when the A280 increased to 5 mAU and the collection is ended when the UV A280 reduced to 5 mAU. Fourth, size exclusion column (SEC) chromatography using Superdex 75 (Cytiva, Marlborugh, MA) is performed to further remove the impurity from the collection of HQ purification. After the SEC column is equilibrated with PBS buffer, the HQ column chromatography collection is loaded. The CD59 protein elution peak is collected when the A280 increased to 5 mAU and the collection is ended when the UV A280 reduced to 5 mAU.

The purified CD59 sample is resolved by SDS-PAGE using 4-12% Bis-Tris gradient gels (Invitrogen™ Novex) according to the manufacturer's instructions. Protein bands are visualized by staining with Coomassie Blue.

vsCD59 N-Terminal Sequencing

The purified vsCD59 proteins are sent for N-terminal sequencing for protein identification. After the vsCD59 proteins are transferred onto the PVDF membrane and stained with SimplyBlue SafeStain (Thermo Fisher Scientific, Cat. #465034), a single band with approximately 8 ug proteins is excised and kept in the 5 mL of Eppendorf tube (data not shown). The bands are thoroughly de-stained by 50%, 60%, and 70% of methanol for 15 min sequentially. Then the blots are washed by milliQ water for 15 min. The blot is air dried overnight. The de-stained membrane is transferred to a fresh tube and stored at 4° C. until the sample is shipped on ice pack to Creative Proteomics (Shirley, NY, USA) for N-terminal first 5 amino acids identification.

vsCD59 Function Assay

To test biological function of vsCD59, cell lysis inhibition assay is performed using Hep1c1c7 cells. The serum dose causing approximately 70% cell lysis is determined for the new normal human serum (NHS) before the purified vsCD59 proteins are used for cell lysis inhibition assay. Three hours prior to the experiment, 1× 10⁴ cells per well are seeded onto a 96-well plate. Various doses of NHS are mixed with GVB2++ buffer (with Ca²⁺ and Mg²⁺) (Complement Technology, Cat. B100). 100 μL of mixture is added to each well. Background lysis is measured using GVB2++ buffer only and maximum lysis is measured by lysing all the cells with Maximum Lysis Buffer (GVB2++ plus 1% SDS). After cells are incubated with mixtures for 1 hour, 10 μL of WST-8 is added into each well of the plate. Again, the cells are incubated with WST-8 for 1-4 hours. The absorbance at 450 nm is measured using a microplate reader. To calculate the percentage of live cells, the absorbance is subtracted by the Max Lysis, divided by Background Lysis minus Max Lysis and multiplied by 100, which equaled the % of live cells. The % of dead cells is calculated by subtracting 100 by the % of live cells. In the GraphPad Prism, the % of cell death is plotted against dose of NHS, and the dose of NHS causing 50% and 70% hemolysis is respectively determined.

Using the NHS dose causing 70% cell lysis, the purified vsCD59 proteins are used for cell lysis inhibition assay. The vsCD59 proteins are 2× serially diluted and mixed with GVB2++ buffer in the presence of NHS, resulting in the final concentration of 30.11, 15.06, 7.53, 3.76, 1.88, and 0.94 μM. The benchmark, CP40 peptide, is also used in this study. The mixture for each sample is added into 4 wells. After quadruple readings is subtracted from the Max Lysis and averaged out, it is divided by 70% of cell lysis minus Max Lysis and multiplied by 100. The % of cell lysis is considered as % of cell lysis inhibition. Using GraphPad Prism software, the percentage of cell lysis inhibition is plotted against the molecular concentration of vsCD59 proteins and IC50 value is estimated.

TABLE 39
DNA sequences of plasmids of Example 13

| Vector | | | |
|---|---|---|---|
| name | Protein | Nucleic acid sequence (ORF for GOI) | SEQ ID NO |
| Vector | (DK) | ATGGAGTTCGGCCTGAGCTGGCTGTTCCTGGTGGCCATC | 304 |
| KRC | vsCD59 | CTTAAGGGCGTGCAGTGCGACAAGCTGCAGTGCTACAAC | |
| | | TGCCCCAACCCCACCGCCGACTGCAAGACCGCCGTGAAC | |
| | | TGCAGCAGCGACTTCGACGCCTGCCTGATCACCAAGGCC | |
| | | GGCCTGCAGGTGTACAACAAGTGCTGGAAGTTCGAGCA | |
| | | CTGCAACTTCAACGACGTGACCACCAGGCTGAGGGAGA | |
| | | ACGAGCTGACCTACTACTGCTGCAAGAAGGACCTGTGCA | |
| | | ACTTCAACGAGCAGCTGGAGAACTAA | |
| Vector | vsCD59 | ATGGGCATCCAGGGCGGCAGCGTGCTGTTCGGCCTGCTG | 305 |
| KRE | | CTGGTGCTGGCCGTGTTCTGCCACAGCGGCCACAGCCTG | |
| | | CAGTGCTACAACTGCCCCAACCCCACCGCCGACTGCAAG | |
| | | ACCGCCGTGAACTGCAGCAGCGACTTCGACGCCTGCCTG | |
| | | ATCACCAAGGCCGGCCTGCAGGTGTACAACAAGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | vsCD59 | ATGGGCATCCAGGGCGGCAGCGTGCTGTTCGGCCTGCTG | 306 |
| KRG | N18Q | CTGGTGCTGGCCGTGTTCTGCCACAGCGGCCACAGCCTG | |
| | | CAGTGCTACAACTGCCCCAACCCCACCGCCGACTGCAAG | |
| | | ACCGCCGTGCAATGCAGCAGCGACTTCGACGCCTGCCTG | |
| | | ATCACCAAGGCCGGCCTGCAGGTGTACAACAAGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | vsCD59 | ATGGGCATCCAGGGCGGCAGCGTGCTGTTCGGCCTGCTG | 307 |
| KRK | Q34E | CTGGTGCTGGCCGTGTTCTGCCACAGCGGCCACAGCCTG | |
| | | CAGTGCTACAACTGCCCCAACCCCACCGCCGACTGCAAG | |
| | | ACCGCCGTGAACTGCAGCAGCGACTTCGACGCCTGCCTG | |
| | | ATCACCAAGGCCGGCCTGGAGGTGTACAACAAGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | vsCD59 | ATGGGCATCCAGGGCGGCAGCGTGCTGTTCGGCCTGCTG | 308 |
| KRM | K38R | CTGGTGCTGGCCGTGTTCTGCCACAGCGGCCACAGCCTG | |
| | | CAGTGCTACAACTGCCCCAACCCCACCGCCGACTGCAAG | |
| | | ACCGCCGTGAACTGCAGCAGCGACTTCGACGCCTGCCTG | |
| | | ATCACCAAGGCCGGCCTGCAGGTGTACAACCGGTGCTGG | |
| | | AAGTTCGAGCACTGCAACTTCAACGACGTGACCACCAGG | |
| | | CTGAGGGAGAACGAGCTGACCTACTACTGCTGCAAGAA | |
| | | GGACCTGTGCAACTTCAACGAGCAGCTGGAGAACCACC | |
| | | ATCACCATCACCATTAA | |
| Vector | vsCD59 | ATGGGCATCCAGGGCGGCAGCGTGCTGTTCGGCCTGCTG | 309 |
| KRP | Q34E, | CTGGTGCTGGCCGTGTTCTGCCACAGCGGCCACAGCCTG | |
| | K38R | CAGTGCTACAACTGCCCCAACCCCACCGCCGACTGCAAG | |
| | | ACCGCCGTGAACTGCAGCAGCGACTTCGACGCCTGCCTG | |
| | | ATCACCAAGGCCGGCCTGGAGGTGTACAACCGGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |
| Vector | vsCD59 | ATGGGCATCCAGGGCGGCAGCGTGCTGTTCGGCCTGCTG | 310 |
| KRQ | N18Q, | CTGGTGCTGGCCGTGTTCTGCCACAGCGGCCACAGCCTG | |
| | K38R | CAGTGCTACAACTGCCCCAACCCCACCGCCGACTGCAAG | |
| | | ACCGCCGTGCAATGCAGCAGCGACTTCGACGCCTGCCTG | |
| | | ATCACCAAGGCCGGCCTGCAGGTGTACAACCGGTGCTGG | |
| | | AAGTTCGAGCACTGCAACTTCAACGACGTGACCACCAGG | |
| | | CTGAGGGAGAACGAGCTGACCTACTACTGCTGCAAGAA | |
| | | GGACCTGTGCAACTTCAACGAGCAGCTGGAGAACCACC | |
| | | ATCACCATCACCATTAA | |
| Vector | vsCD59 | ATGGGCATCCAGGGCGGCAGCGTGCTGTTCGGCCTGCTG | 311 |
| KRR | N18Q, | CTGGTGCTGGCCGTGTTCTGCCACAGCGGCCACAGCCTG | |
| | Q34E, | CAGTGCTACAACTGCCCCAACCCCACCGCCGACTGCAAG | |
| | K38R | ACCGCCGTGCAATGCAGCAGCGACTTCGACGCCTGCCTG | |
| | | ATCACCAAGGCCGGCCTGGAGGTGTACAACCGGTGCTG | |
| | | GAAGTTCGAGCACTGCAACTTCAACGACGTGACCACCAG | |
| | | GCTGAGGGAGAACGAGCTGACCTACTACTGCTGCAAGA | |
| | | AGGACCTGTGCAACTTCAACGAGCAGCTGGAGAACCAC | |
| | | CATCACCATCACCATTAA | |

TABLE 40
Peptides and fusion protein sequences of Example 13

| Vector | | | |
|---|---|---|---|
| name | Protein | Nucleic acid sequence (ORF for GOI) | SEQ ID NO |
| Vector | Vh(DK) | MEFGLSWLFLVAILKGVQCDKLQCYNCPNPTADCKT | 312 |
| KRC | vsCD59 | AVNCSSDFDACLITKAGLQVYNKCWKFEHCNFNDVT | |
| | | TRLRENELTYYCCKKDLCNFNEQLEN | |
| Vector | vsCD59- | MGIQGGSVLFGLLLVLAVFCHSGHSLQCYNCPNPTAD | 313 |
| KRE | 6 × His | CKTAVNCSSDFDACLITKAGLQVYNKCWKFEHCNFN | |
| | | DVTTRLRENELTYYCCKKDLCNFNEQLENHHHHHH | |
| Vector | vsCD59 | MGIQGGSVLFGLLLVLAVFCHSGHSLQCYNCPNPTAD | 314 |
| KRG | N18Q- | CKTAVQCSSDFDACLITKAGLQVYNKCWKFEHCNFN | |
| | 6 × His | DVTTRLRENELTYYCCKKDLCNFNEQLENHHHHHH | |
| Vector | vsCD59 | MGIQGGSVLFGLLLVLAVFCHSGHSLQCYNCPNPTAD | 315 |
| KRK | Q34E- | CKTAVNCSSDFDACLITKAGLEVYNKCWKFEHCNFN | |
| | 6 × His | DVTTRLRENELTYYCCKKDLCNFNEQLENHHHHHH | |
| Vector | vsCD59 | MGIQGGSVLFGLLLVLAVFCHSGHSLQCYNCPNPTAD | 316 |
| KRM | K38R- | CKTAVNCSSDFDACLITKAGLQVYNRCWKFEHCNFN | |
| | 6 × His | DVTTRLRENELTYYCCKKDLCNFNEQLENHHHHHH | |
| Vector | vsCD59 | MGIQGGSVLFGLLLVLAVFCHSGHSLQCYNCPNPTAD | 317 |
| KRP | Q34E, | CKTAVNCSSDFDACLITKAGLEVYNRCWKFEHCNFN | |
| | K38R- | DVTTRLRENELTYYCCKKDLCNFNEQLENHHHHHH | |
| | 6 × His | | |
| Vector | vsCD59 | MGIQGGSVLFGLLLVLAVFCHSGHSLQCYNCPNPTAD | 318 |
| KRQ | N18Q, | CKTAVQCSSDFDACLITKAGLQVYNRCWKFEHCNFN | |
| | K38R- | DVTTRLRENELTYYCCKKDLCNFNEQLENHHHHHH | |
| | 6 × His | | |
| Vector | vsCD59 | MGIQGGSVLFGLLLVLAVFCHSGHSLQCYNCPNPTAD | 319 |
| KRR | N18Q, | CKTAVQCSSDFDACLITKAGLEVYNRCWKFEHCNFN | |
| | Q33E, | DVTTRLRENELTYYCCKKDLCNFNEQLENHHHHHH | |
| | K38R- | | |
| | 6 × His | | |

### Example 14. Cynomolgus Monkey In Vivo Expression Assay

The objective of this study is to evaluate the possible effects of constructs following a single intravitreal injection prior to sodium iodate (SI) induction of retinal degeneration in Cynomolgus monkeys (Macaca fascicularis). AAV constructs Vector KMR, Vector KKT, Vector KPP are used for intravitreal injection. Vector EKQ with EGFP gene is used as transfection control and Syfovre is used as a comparison control. The AAV-originated plasmids have dual cassettes. In the first cassette, all contained sCD59 with promoters in addition to a different expression elements. In the second cassette, they have different gene of interest, for example, Vector KMR has C3i(N15Q)-Fc4-C3i(N15Q), Vector KKT has C3i(N15Q)-Fc4-CNP36, and Vector KPP has C3i(N15Q)-Fc4-Endostatin.

Sixteen male monkeys are divided into 6 groups (Table 41). Groups 1, 2, 4, 5, and 6 are dosed 28 days prior to modeling with sodium iodate. Before the intravitreal injection, animals receive mydriatic agent first and then anesthetized using Zoletil® 50 (8 mg/kg, 50 mg/mL) by intramuscular injection, the supplementary dose can be provided according to SOP (TOX-039). The most current body weights are used for calculation of the anesthetics. The animal are kept warm using appropriate means (for example, covering blanket) until regaining consciousness and cared by a designated person during anesthesia. Monkeys are dosed according to Table 41. All animals in Groups 1 to 6 will receive levofloxacin eye drops and/or ofloxacin eye ointment 2 to 3 times daily for 3 consecutive days after administration.

TABLE 41 [symbol]
Study Design Dosage

| | | | | Dose | Number of |
|---|---|---|---|---|---|
| Group | | Dose Level | | Volume | Animals |
| 1 | Vehicle Control Group | 0 | | 50 μL/eye | 3 |
| 2 | Vector EKQ Group | 1 × 10¹¹ | vg/eye | 50 μL/eye | 1 |
| 3 | Control Article Group | 7.5 | mg/eye | 50 μL/eye | 3 |
| 4 | Vector KKT Group | 1 × 10¹¹ | vg/eye | 50 μL/eye | 3 |
| 5 | Vector KMR Group | 1 × 10¹¹ | vg/eye | 50 μL/eye | 3 |
| 6 | Vector KPP Group | 1 × 10¹¹ | vg/eye | 50 μL/eye | 3 |

Next, the animal model is established via injection of sodium iodate. The neck of anesthetized animals is unhaired and disinfected. The skin on the right side of the larynx is cut and the muscles and soft tissues are separated bluntly. The internal and external carotid arteries after branches are found along the common carotid artery. The common and external carotid arteries are clamped with arterial clamp. Sodium iodate (20 mg/kg, 10 mg/mL) is injected through the bifurcation of the common carotid artery, and the common carotid artery blood flow is blocked for 1 min. The needle is pulled out quickly, hemostatic cotton and dry cotton ball or gauze is used for hemostasis, and the common carotid artery and external carotid artery clips are released. The muscle and skin is sutured when the carotid stops bleeding. The surgical wound is disinfected and sprayed with ceftriaxone sodium. If the procedure fails on the right carotid artery, the SI induction at the left side of the same animal is performed. Ceftriaxone sodium (diluted with normal saline to 250 mg/mL) is intramuscularly injected (80 mg/kg, once a day) for 3 consecutive days after surgery. Ceftriaxone sodium is sprayed.

### Clinical Observations

All animals in Groups 1 to 6 are examined closely for clinical signs within the week after modeling. The observed findings in body surface and visible mucosa changes, neurological and behavioral activities, respiratory manifestations, gastrointestinal reactions, urinary and reproductive orifices are recorded.

Body weights are obtained from all animals in Groups 1 to 6 on the screening day, Day-28 (only Group 1, 2, 4, 5, 6), Day-3, Day 1 (pre-dose), Day 8, Day 15, Day 29 and Day 60 during the study period. The body weight gains in Groups 1 to 6 is recorded. Body weight is taken prior to the scheduled necropsy. Body weight is be recorded when an animal is found dead or euthanized in extremis in Groups 1 to 6.

General ocular examination is conducted for animals in Groups 1, 2, 4, 5 and 6 prior to dosing (Day-28), Day-21, Day-14. General ocular examination is conducted for animals in Groups 1 to 6 on Day-3, and Days 8, 15, 29 and 60. Anterior segment of the eyes: the eyelid, cornea, sclera, conjunctiva, anterior chamber, iris, pupil, lens, vitreous body, and etc. are observed carefully using the slit lamp (KOWA SL-17, Kowa Company, Ltd., Japan; and/or PSL CLASSIC, Keeler. Co. UK, United Kingdom; and/or SLM-6H, KANGHUA RUIMING S&T CO., LTD, China; and/or SLM-8E, CHONGQING KANGHUA RUIMING S&T CO., LTD). Photos of any abnormalities are taken. The fundus is examined using a direct ophthalmoscope (HEINE mini 3000®, HEINE Optotechnik GmbH & Co. KG, Germany). The degree of ocular abnormalities (+, +, ++, +++, ++++ indicating degrees of traceable, minimal, slight, moderate and severe, respectively) is graded using the SPOTS scoring system (Eaton, Seth, J., 2017), and findings without scoring criteria in the SPOTS scoring system are described by the Study Ophthalmologist using standardized terminology.

Intraocular pressure is recorded for animals in Groups 1, 2, 4, 5 and 6 on Day −28 (prior to dosing) and Days −21, −14. All animals in Groups 1 to 6 intraocular pressure is measured on Days −3, 8, 15, 29 and 60. Intraocular pressure is measured using a Model 30 Pneumatic Tonometer (Model 30™, Reichert Technologies, USA).

Dark-adapted and light-adapted electroretinography (ERG) is conducted for animals in Groups 1, 2, 4, 5 and 6 on Days −21, −14 and −3 using Ophthalmic Electrophysiological Diagnosis System (RETI-Scan 21, ROLAND CONSULT Stasche & Finger GmbH, Germany). All animals in Groups 1 to 6 are measured on Days 8, 15, 29 and 60. Animals are placed in a dark room for at least 30 minutes for dark adaptation prior to dark-adapted ERG testing (including dark-adapted 0.01 ERG, dark-adapted 3.0 ERG+Ops and dark-adapted 10.0 ERG). After the dark-adapted ERG testing, animals are placed in a light room for at least 10 minutes for light adaptation prior to light-adapted ERG testing (including light-adapted 3.0 ERG, and light-adapted 3.0 scintillation ERG).

Spectral Domain-Optical coherence tomography (SD-OCT) is conducted for animals in Groups 1, 2, 4, 5 and 6 on Days −21, −14, −3 using Spectralis® HRA OCT (Heidelberg Engineering, Germany). All animals in Groups 1 to 6 are measured on Days 8, 15, 29 and 60. Images are taken covering five areas of the fundus including superior vascular arch, inferior vascular arch, temporal, nasal (including optic disc), and macular area. The scanned area in each eye has a field of view around 55-degree. The thickness of the retina in OCT images is determined using the instrument software.

Blue autofluorescence (BAF) is conducted for animals in Groups 1, 2, 4, 5 and 6 on Days −21, −14, −3 using Spectralis® HRA OCT (Heidelberg Engineering, Germany). All animals in Groups 1 to 6 are measured on Days 8, 15, 29 and 60. Images are taken covering five areas of the fundus including superior vascular arch, inferior vascular arch, temporal, nasal (including optic disc), and macular area. The scanned area in each eye has a field of view around 55-degree.

Fundus fluorescein angiography (FFA) and fundus photography (FP) are conducted for animals in Groups 1, 2, 4, 5 and 66 on Days −21, −14, −3 using Digital Fundus Angiography (APS-BER, CHONGQING KANGHUA RUIMING S&T CO., LTD). All animals in Groups 1 to 6 are measured on Days 8, 15, 29 and 60. The scanned area in each eye has a field of view around 53-degree. Five regions of the fundus (superior vascular arch, inferior vascular arch, temporal, nasal (including optic disc), and macular area) are photographed. Early images are taken within about 1.5 min with fluorescein angiography, and late images are taken after about 5 min. Animals are given fluorescein sodium injection by intravenous injection (8 to 10 mg/kg, 100 mg/mL) before the fluorescein angiography.

Aqueous humor samples are collected for each monkey in Groups 1, 2, 3, 4, 5 and 6 on Days −14. All animals in Groups 1 to 6 are collected on Days 29 and 60. On Days −14 and 29, 20 μL aqueous humor samples are collected and mixed with 180 μL PBS dilution buffer (JOINN will provide sterile 1×PBS) with 1% BSA (C134730100, Acros). On Day 60, the aqueous humor is collected.

Blood samples of approximately 2.0-3.0 mL are collected from all animals in all Groups via forelimb or hindlimb subcutaneous vein on Days −14. All animals in Groups 1 to 6 are collected on Days −1, 29 and 60. Blood samples are collected into tubes containing separation gel and coagulant. The whole blood samples are stored and transported at room temperature and centrifuged at 1500×g for 10 min at 2 to 8° C. within 2 hours after blood collection. After centrifugation, the collected supernatant (serum) is transferred to a new labeled centrifuge tube and stored at ≤−60° C. Blood sterile samples of approximately 1.5-2.0 mL is collected from all animals in Groups 1 to 6 via forelimb or hindlimb subcutaneous vein into a labeled collection tube containing EDTA-K2 anticoagulant on Days −14. All animals in Groups 1 to 6 are collected on Days −1, 29 and 60. The tubes are stored on crushed ice before collection and stored and transported on dry ice temporarily after collection. Anticoagulated whole blood samples are dispensed as about 500 μL/tube within 2 hours, transferred into a new labeled centrifuge tube, and stored at ≤−60° C.

All monkeys receive a complete macroscopic examination. Animals found dead after regular working hours are refrigerated (2 to 8° C.) and necropsies are performed as soon as possible within 24 hours. Carcasses are disposed of as medical waste following macroscopic examination and requested tissue collection. The macroscopic examination includes the external surfaces of the body, all orifices of the body, and the cranial cavity, thoracic cavity, abdominal cavity as well as their contents.

Both eyes of animal in Group 2 are collected at scheduled necropsy on Day 60. Both eyes are soaked in 10% neutral buffered formalin (NaH₂PO₄(2H₂0), Na₂HPO₄(12H₂0) and 36-40% formaldehyde solution, pH is 6.8-7.2) for Immunohistochemistry (IHC) using anti-GFP, anti-RPE65, PNA Lectin Cy5 and DAPI antibodies. Modeling eyes in Group 1 and Groups 3 to 6 (in group order) are collected at scheduled necropsy on Day 60. The aqueous humor, vitreous humor, retina, optic nerve (including posterior orbital optic nerve), optic chiasm, optic tract, lateral geniculate nucleus (LGN), and visual cortex are separated. Each tissue is collected and aliquoted into RNase-free tubes for two sets (100 mg or 0.1 to 0.2 mL of aqueous humor and vitreous humor for one per set of samples, depending on the size of the tissue). The tissues from left eye are used for protein analysis and the tissues from right eyes are used for DNA/RNA analyses. All samples are stored in dry ice temporarily, and then stored below −60° C. One set of anatomical equipment is used for each side of each animal.

The main organs are collected, including brain (cerebral cortex, cerebellum), reproductive system (testicles), colon, spinal cord (cervical, thoracic, lumbar, sacral), submandibular lymph nodes, mesenteric lymph nodes, heart, lungs, adrenal gland, kidneys, liver and spleen at scheduled necropsy in Group 1 and Group 3 to 6 (in group order) on Day 60. Three or Four sets of each tissue (one per set of samples is 100 mg) is collected into RNase-free tubes. For paired organs (eg. lung, kidney etc), left is used for protein analysis and right is used for DNA/RNA analyses. All samples are stored in dry ice temporarily, and then stored below −60° C.

All statistical tests are conducted as 2-sided tests with a significance level set at 0.05 or P≤0.05. Group means and standard deviations (SD) are calculated for IOP and ERG by SPSS 13.0. The data are analyzed with the following procedures: A Levene's test is performed on the original data to test for variance homogeneity. If the result shows no significance (P>0.05), a one-way analysis of variance (ANOVA) is performed. If ANOVA shows significance (P≤0.05), multiple comparisons is performed. In the case of variance heterogeneity (P≤0.05 by Levene's test), a Kruskal-Wallis test (non-parametric method) is performed on the original data. If Kruskal-Wallis test shows significance (P≤0.05), a two-independent-sample test (Mann-Whitney U) is performed for multiple comparisons.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1.-50. (canceled)

51. A method of treating a disease or condition in a subject, the method comprising administering to the subject an engineered polynucleotide comprising one or more expression cassettes, the one or more expression cassettes encoding a first angiogenesis inhibitor and a second angiogenesis inhibitor.

52. An engineered polynucleotide encoding an inhibitor of a membrane attack complex (MAC) and a natriuretic peptide.

53. The engineered polynucleotide of claim 52, wherein the inhibitor of a membrane attack complex (MAC) comprises a CD59.

54. The engineered polynucleotide of claim 53, wherein the CD 59 comprises an amino acid sequence that is at least 80% identical to any one of SEQ ID NOs: 41-45, 312-319, or 325-329.

55. The engineered polynucleotide of claim 52, wherein the natriuretic peptide is covalently connected to an antibody or fragment thereof.

56. The engineered polynucleotide of claim 55, wherein the antibody or fragment thereof comprises a fragment crystallizable (Fc) region.

57. The engineered polynucleotide of claim 52, wherein the natriuretic peptide comprises a C-type natriuretic peptide (CNP).

58. The engineered polynucleotide of claim 52, wherein the natriuretic peptide comprises an amino acid sequence that is at least 80% identical to any one of SEQ ID NOs: 61-72.

59. The engineered polynucleotide of claim 58, wherein the natriuretic peptide consists the amino acid sequence that is any one of SEQ ID NOs: 61-72.

60. The engineered polynucleotide of claim 52, wherein the natriuretic peptide is covalently connected to a complement 3 inhibitor or a C3 degraded fragment.

61. The engineered polynucleotide of claim 60, wherein the C3 degraded fragment comprises a C3a, C3b, iC3b, C3f, C3c, C3d, C3g, or a combination thereof.

62. The engineered polynucleotide of claim 60, wherein the complement 3 inhibitor comprises an amino acid sequence that is at least 80% identical to any one of SEQ ID NOs: 1-15.

63. The engineered polynucleotide of claim 62, wherein the complement 3 inhibitor consists the amino acid sequence that is any one of SEQ ID NOs: 1-15.

64. The engineered polynucleotide of claim 52, wherein the engineered polynucleotide comprises a viral vector.

65. The engineered polynucleotide of claim 64, wherein the viral vector comprises an AAV vector.

66. The engineered polynucleotide of claim 65, wherein the AAV vector is an AAV2 vector.

67. The engineered polynucleotide of claim 66, wherein the AAV vector encodes an engineered AAV capsid.

68. The engineered polynucleotide of claim 52, wherein the engineered polynucleotide encodes a protease peptide sequence flanked by the inhibitor of a membrane attack complex (MAC) and the natriuretic peptide.

69. A pharmaceutical composition comprising the engineered polynucleotide of claim 52.

70. A method of treating a disease or condition in a subject, comprising administering to the subject the engineered polynucleotide of claim 52.


SEQUENCE LISTING (337 sequences)

SEQ ID NO: 1 - protein, 14 aa, synthetic construct
GICVVQDWGHHRCT

SEQ ID NO: 2 - protein, 15 aa, synthetic construct
GICVWQDWGAHRCTN

SEQ ID NO: 3 - protein, 15 aa, synthetic construct
GICVWQDWGAHRCAN

SEQ ID NO: 4 - protein, 16 aa, synthetic construct
GYICVWQDWGAHRCTN

SEQ ID NO: 5 - protein, 16 aa, synthetic construct
GYICVWQDWGAHRCAN

SEQ ID NO: 6 - protein, 14 aa, synthetic construct
GICVWQDWGAHRCT

SEQ ID NO: 7 - protein, 14 aa, synthetic construct
GICVWQDWGAHRCA

SEQ ID NO: 8 - protein, 15 aa, synthetic construct
GYICVWQDWGAHRCT

SEQ ID NO: 9 - protein, 15 aa, synthetic construct
GYICVWQDWGAHRCA

SEQ ID NO: 10 - protein, 15 aa, synthetic construct
GICVWQDWGAHRCTQ

SEQ ID NO: 11 - protein, 15 aa, synthetic construct
GICVWQDWGAHRCAQ

SEQ ID NO: 12 - protein, 16 aa, synthetic construct
GYICVWQDWGAHRCTQ

SEQ ID NO: 13 - protein, 16 aa, synthetic construct
GYICVWQDWGAHRCAQ

SEQ ID NO: 14 - protein, 27 aa, synthetic construct
DKGICVWQDWGAHRCTNGGGGSGGGGS

SEQ ID NO: 15 - protein, 29 aa, synthetic construct
EVQLGICVWQDWGAHRCTNGGGGSGGGGS

SEQ ID NO: 16-19 - skipped (4 entries)

SEQ ID NO: 20 - DNA, 81 bp, synthetic construct
  source 1..81: mol_type = other DNA
gacaagggcatctgcgtgtggcaggactggggcgcccaccgctgcaccaacggcggcggcggcagcggcggcggcggcagc

SEQ ID NO: 21 - DNA, 81 bp, synthetic construct
  source 1..81: mol_type = other DNA
gacaagggcatctgcgtgtggcaggactggggcgcccaccgctgcaccaacggcggaggcggaagcggcggaggcggatct

SEQ ID NO: 22 - DNA, 45 bp, synthetic construct
  source 1..45: mol_type = other DNA
ggcatctgcgtgtggcaggactggggcgcccaccgctgcaccaac

SEQ ID NO: 23 - DNA, 45 bp, synthetic construct
  source 1..45: mol_type = other DNA
ggcatctgcgtgtggcaggactggggcgcccaccgctgcgccaac

SEQ ID NO: 24 - DNA, 48 bp, synthetic construct
  source 1..48: mol_type = other DNA
ggctacatctgcgtgtggcaggactggggcgcccaccgctgcaccaac

SEQ ID NO: 25 - DNA, 48 bp, synthetic construct
  source 1..48: mol_type = other DNA
ggctacatctgcgtgtggcaggactggggcgcccaccgctgcgccaac

SEQ ID NO: 26 - DNA, 42 bp, synthetic construct
  source 1..42: mol_type = other DNA
ggcatctgcgtgtggcaggactggggcgcccaccgctgcacc

SEQ ID NO: 27 - DNA, 42 bp, synthetic construct
  source 1..42: mol_type = other DNA
ggcatctgcgtgtggcaggactggggcgcccaccgctgcgcc

SEQ ID NO: 28 - DNA, 45 bp, synthetic construct
  source 1..45: mol_type = other DNA
ggctacatctgcgtgtggcaggactggggcgcccaccgctgcacc

SEQ ID NO: 29 - DNA, 45 bp, synthetic construct
  source 1..45: mol_type = other DNA
ggctacatctgcgtgtggcaggactggggcgcccaccgctgcgcc

SEQ ID NO: 30 - DNA, 45 bp, synthetic construct
  source 1..45: mol_type = other DNA
ggcatctgcgtgtggcaggactggggcgcccaccgctgcacccag

SEQ ID NO: 31 - DNA, 45 bp, synthetic construct
  source 1..45: mol_type = other DNA
ggcatctgcgtgtggcaggactggggcgcccaccgctgcgcccag

SEQ ID NO: 32 - DNA, 48 bp, synthetic construct
  source 1..48: mol_type = other DNA
ggctacatctgcgtgtggcaggactggggcgcccaccgctgcacccag

SEQ ID NO: 33 - DNA, 48 bp, synthetic construct
  source 1..48: mol_type = other DNA
ggctacatctgcgtgtggcaggactggggcgcccaccgctgcgcccag

SEQ ID NO: 34-40 - skipped (7 entries)

SEQ ID NO: 41 - protein, 128 aa, synthetic construct
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 42 - protein, 102 aa, synthetic construct
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLEN

SEQ ID NO: 43 - protein, 103 aa, synthetic construct
LQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 44 - protein, 77 aa, synthetic construct
LQCYNCPNPTADCKTAVNCSSDFDACLITKAGLQVYNKCWKFEHCNFNDVTTRLRENELTYYCCKKDLCNFNEQLEN

SEQ ID NO: 45 - protein, 80 aa, synthetic construct
EVQLQCYNCPNPTADCKTAVNCSSDFDACLITKAGLQVYNKCWKFEHCNFNDVTTRLRENELTYYCCKKDLCNFNEQLEN

SEQ ID NO: 46-50 - skipped (5 entries)

SEQ ID NO: 51 - protein, 184 aa, synthetic construct
MHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 52-60 - skipped (9 entries)

SEQ ID NO: 61 - protein, 126 aa, synthetic construct
MHLSQLLACALLLTLLSLRPSEAKPGAPPKVPRTPPAEELAEPQAAGGGQKKGDKAPGGGGANLKGDRSRLLRDLRVDTKSRAAWARLLQEHPNARKYKGANKKGLSKGCFGLKLDRIGSMSGLGC

SEQ ID NO: 62 - protein, 61 aa, synthetic construct
GDRSRLLRDLRVDTKSRAAWARLLQEHPNARKYKGANKKGLSKGCFGLKLDRIGSMSGLGC

SEQ ID NO: 63 - protein, 53 aa, synthetic construct
DLRVDTKSRAAWARLLQEHPNARKYKGANKKGLSKGCFGLKLDRIGSMSGLGC

SEQ ID NO: 64 - protein, 36 aa, synthetic construct
EHPNARKYKGANKKGLSKGCFGLKLDRIGSMSGLGC

SEQ ID NO: 65 - protein, 22 aa, synthetic construct
GLSKGCFGLKLDRIGSMSGLGC

SEQ ID NO: 66 - protein, 19 aa, synthetic construct
MEFGLSWLFLVAILKGVQC

SEQ ID NO: 67 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 233 aa, synthetic construct
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - protein, 54 aa, synthetic construct
MEFGLWLFLVAILKGVQCEHPNARKYKGANKKGLSKGCFGLKLDRIGSMSGLGC

SEQ ID NO: 70 - protein, 40 aa, synthetic construct
MEFGLWLFLVAILKGVQCGLSKGCFGLKLDRIGSMSGLGC

SEQ ID NO: 71 - protein, 302 aa, synthetic construct
MEFGLSWLFLVAILKGVQCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEHPNARKYKGANKKGLSKGCFGLKLDRIGSMSGLGC

SEQ ID NO: 72 - protein, 287 aa, synthetic construct
MEFGLSWLFLVAILKGVQCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSLSKGCFGLKLDRIGSMSGLGC

SEQ ID NO: 73-80 - skipped (8 entries)

SEQ ID NO: 81 - protein, 123 aa, synthetic construct [sequence omitted]

SEQ ID NO: 82 - protein, 112 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAA

SEQ ID NO: 83 - protein, 255 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSGGGGSGGGGSGGGGSGGGGSDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAA

SEQ ID NO: 84 - protein, 231 aa, synthetic construct
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 85 - protein, 214 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 255 aa, synthetic construct
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAGGGGSGGGGSGGGGSGGGGSEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSS

SEQ ID NO: 87 - protein, 252 aa, synthetic construct
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 88 - protein, 467 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 204 aa, synthetic construct
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEK

SEQ ID NO: 90 - protein, 431 aa, synthetic construct
DTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 431 aa, synthetic construct
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 457 aa, synthetic construct
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 93-100 - skipped (8 entries)

SEQ ID NO: 101 - DNA, 8678 bp, synthetic construct
  source 1..8678: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggggtaccgggccccccctcgaggtcgacggtatcgatggatccgatatcgaattcaagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacagagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatggagttcggcctgagctggctgttcctggtggccatccttaagggcgtgcagtgcgaggtgcagctgggcatctgcgtgtggcaggactggggcgcccaccgctgcaccaacggcggaggcggaagcggcggaggcggatctggcggaggcggaagcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgctggtaccgggccccccctcgaggtcgacggtatcgatggatccgatatcgaattcaacctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacggttactcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcc

SEQ ID NO: 102 - DNA, 5153 bp, synthetic construct
  source 1..5153: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagagcttacattattgaagcatttatcagggttattgtctcagacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgtaccggttcgaacaggtaagcgcccctaaaatccctttgggcacaatgtgtcctgaggggagaggcagcgacctgtagatgggacgggggcactaaccctcaggtttggggcttctgaatgtgagtatcgccatgtaagcccagtatttggccaatctcagaaagctcctggtccctggagggatggagagagaaaaacaaacagctcctggagcagggagagtgctggcctcttgctctccggctccctctgttgccctctggtttctccccaggttcgaagcgcgcaattaaccctcactaaagggaacaaaagctggagctcaatcggaccgaaattaatacgactcactataggggaattgtgagcggataacaattccccggagttaatccgggacctttaattcaacccaacacaatatattatagttaaataagaattattatcaaatcatttgtatattaattaaaatactatactgtaaattacattttatttacaatcaaaggagatataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccggccgcacagctgtatacacgtgcaagccagccagaactcgccccggaagaccccgaggatctcgagcaccaccatcaccatcaccatcactaagtgattaacctcaggtgcaggctgcctatcagaaggtggtggctggtgtgggtacccaattcgccctatagtgagtcgtattacgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctccggacacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcatgcaagcgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatggatcctgtctcaaaatctctgatgttacattgcacaagataaaaatatatcatcatgaacaataaaactgtctgcttacataaacagtaatacaaggggtgttatgagccatattcaacgggaaacgtcttgctcaaggccgcgattaaattccaacatggatgctgatttatatgggtataaatgggctcgcgataatgtcgggcaatcaggtgcgacaatctatcgactttatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcgcctaggctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgaattcaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 103 - DNA, 7014 bp, synthetic construct
  source 1..7014: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggctcaagcagtgatcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagtacttctcgacaagctcggatcctgcgcgctcgctcgctcactgaggccgcccgggcaaagcccgggcgtcgggcgacctttggtcgcccggcctcagtgagcgagcgagcgcgcagagagggagtggggttgatctctccccagcatgccacacaaaaaaccaacacacagatgtaatgaaaataaagatattttattttcgaaggtaccaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattctcgagttaacatcccaggccgctcatggagccgattcggtccagcttgaggccgaagcagcccttggacaagcccttcttgttggctcctttgtatttgcgcgcgttggggtgctcagagccgcctccgccgcttccgcctccgccagatccgcctccgccgcttccgcctccgcccttgcccaggctcaggctcaggctcttctgggtgtagtggttgtgcagggcctcgtgcatcacgctgcagctgaacacgttgccctcctgccatctgctcttgtccacggtcagtctgctgtacaggaagaagctgccgtcgctgtccagcacggggggggtggtcttgtagttgttctcgggctggccgttgctctcccactccacggcgatgtcgctggggtagaagcccttcaccaggcaggtcaggctcacctggttcttggtcatctcctcctggctggggggcagggtgtacacctggggctctctgggctggcccttggccttgctgatggtcttctcgatgctgctgggcaggcccttgttgctcaccttgcacttgtactccttgccgttcagccagtcctggtgcagcacggtcagcacgctcaccactctgtaggtgctgttgaactgctcctctctgggcttggtcttggcgttgtgcacctccacgccgtccacgtaccagttgaactgcacctcggggtcctcctggctcacgtccaccaccacgcaggtcacctcgggggttctgctgatcatcagggtgtccttgggcttgggggggaacaggaacacgctggggccgcccaggaactcgggggcggggcagggggggcagggggggccgtacttgctctccactctcttgtcgcactgcacgcccttaaggatggccaccaggaacagccagctcaggccgaactccatggtggcggcaggcctagaagtaaaggcaacatccactgaggagcagttctttgatttgcaccaccaccggatccgggacctgaaataaaagacaaaaagactaaacttacctgtgggagtaacgcggtcagtcagagccggggcgggcggcgcgaggcggcggcggagcggggcacggggcgaaggcagcgtcgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccccgcccccatcgctgcacaaaataattaaaaaataaataaatacaaaattgggggtggggaggggggggagatggggagagtgaagcagaacgtggggctcacctcgagccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggtaccgaattcagatcagcttcctaggaacccctagtgatggagttggccactccctctctgcgcgctcgctcgctcactgaggccgggcgaccaaaggtcgcccgacgcccgggctttgcccgggcggcctcagtgagcgagcgagcgcgccaggatccgagcttgtcgagaagtactagaggatcatcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtc

SEQ ID NO: 104-110 - skipped (7 entries)

SEQ ID NO: 111 - protein, 323 aa, synthetic construct
EVQLGICVWQDWGAHRCTNGGGGSGGGGSGGGGSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEHPNARKYKGANKKGLSKGCFGLKLDRIGSMSGLGC

SEQ ID NO: 112 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 113 - protein, 289 aa, synthetic construct
DKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEHPNARKYKGANKKGLSKGCFGLKLDRIGSMSGLGC

SEQ ID NO: 114-120 - skipped (7 entries)

SEQ ID NO: 121 - DNA, 6869 bp, synthetic construct
  source 1..6869: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccttgctgtgtaacgtcattgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtaagtttagtctttttgtcttttatttcaggtcccggatccggtggtggtgcaaatcaaagaactgctcctcagtggatgttgcctttacttctaggcctgccgccaccatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtggtaccttcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcaggatccgagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 122 - DNA, 6869 bp, synthetic construct
  source 1..6869: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccttgctgtgtaacgtcattgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtaagtttagtctttttgtcttttatttcaggtcccggatccggtggtggtgcaaatcaaagaactgctcctcagtggatgttgcctttacttctaggcctgccgccaccatgggcatccagggcggcagcgtgctgttcggcctgctgctggtgctggccgtgttctgccacagcggccacagcctgcagtgctacaactgccccaaccccaccgccgactgcaagaccgccgtgaactgcagcagcgacttcgacgcctgcctgatcaccaaggccggcctgcaggtgtacaacaagtgctggaagttcgagcactgcaacttcaacgacgtgaccaccaggctgagggagaacgagctgacctactactgctgcaagaaggacctgtgcaacttcaacgagcagctggagaacggcggcaccagcctgagcgagaagaccgtgctgctgctggtgacccccttcctggccgccgcctggagcctgcacccctgaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtggtaccttcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcaggatccgagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 123 - DNA, 6869 bp, synthetic construct
  source 1..6869: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccttgctgtgtaacgtcattgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtaagtttagtctttttgtcttttatttcaggtcccggatccggtggtggtgcaaatcaaagaactgctcctcagtggatgttgcctttacttctaggcctgccgccaccatgggcatccagggcggcagcgtgctgttcggcctgctgctggtgctggccgtgttctgccacagcggccacagcctgcagtgctacaactgccccaaccccaccgccgactgcaagaccgccgtgaactgcagcagcgacttcgacgcctgcctgatcaccaaggccggcctgcaggtgtacaacaagtgctggaagttcgagcactgcaacttcaacgacgtgaccacccgcctgcgcgagaacgagctgacctactactgctgcaagaaggacctgtgcaacttcaacgagcagctggagaacggcggcaccagcctgagcgagaagaccgtgctgctgctggtgacccccttcctggccgccgcctggagcctgcacccctaaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtggtaccttcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcaggatccgagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 124 - DNA, 6869 bp, synthetic construct
  source 1..6869: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccttgctgtgtaacgtcattgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtaagtttagtctttttgtcttttatttcaggtcccggatccggtggtggtgcaaatcaaagaactgctcctcagtggatgttgcctttacttctaggcctgccgccaccatgggaatccaaggcggctctgtgctgtttggcctgctgctggttctggccgtgttttgtcactctggccacagcctgcagtgctacaactgccctaatcctaccgccgactgcaagaccgccgtgaactgcagcagcgatttcgacgcctgcctgatcacaaaggccggactgcaggtctacaacaagtgctggaagttcgagcactgcaacttcaacgacgtgaccaccagactgagagagaacgagctgacctactactgctgcaagaaagacctgtgcaatttcaatgagcagctggaaaacggcggcaccagcctgagcgaaaagacagtgctgctgctcgtgacccctttcctggctgctgcttggagcctgcatccttgaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtggtaccttcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcaggatccgagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 125 - DNA, 6869 bp, synthetic construct
  source 1..6869: mol_type = other DNA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccttgctgtgtaacgtcattgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgctgcgcgctcgctcgctcactgaggccgcccgggcaaagcccgggcgtcgggcgacctttggtcgcccggcctcagtgagcgagcgagcgcgcagagagggagtggccaactccatcactaggggttcctaggaagctgatctgaattcggtacccgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtaagtttagtctttttgtcttttatttcaggtcccggatccggtggtggtgcaaatcaaagaactgctcctcagtggatgttgcctttacttctaggcctgccgccaccatgggcatccagggcggcagcgtgctgtttggactgctgctggtcctcgccgtgttctgccacagcggccattctctgcaatgttataactgtcctaacccaacagctgattgcaaaacagccgtgaactgcagctctgatttcgacgcctgcctgatcaccaaggccggcctgcaggtgtacaacaagtgctggaagttcgagcactgtaattttaacgacgtgaccaccagactgcgggaaaatgagctgacctactactgctgcaaaaaggacctgtgcaacttcaacgagcagctggaaaacggcggaaccagcctgagcgagaagaccgtgctgctgctggtgacacctttcctggccgccgcttggtccctgcacccctaaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtggtaccttcgaaaataaaatatctttattttcattacatctgtgtgttggttttttgtgtggcatgctggggagagatcaaccccactccctctctgcgcgctcgctcgctcactgaggccgggcgaccaaaggtcgcccgacgcccgggctttgcccgggcggcctcagtgagcgagcgagcgcgcaggatccgagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 126 - DNA, 6869 bp, synthetic construct; identical to SEQ ID NO: 125
  source 1..6869: mol_type = other DNA

SEQ ID NO: 127 - DNA, 7949 bp, synthetic construct
  source 1..7949: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggggtaccgggccccccctcgaggtcgacggtatcgatggatccgatatcgaattcaagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacagagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatggagttcggcctgagctggctgttcctggtggccatccttaagggcgtgcagtgcgaggtgcagctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaattaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgctggtaccgggccccccctcgaggtcgacggtatcgatggatccgatatcgaattcaacctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacggttactcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcc

SEQ ID NO: 128 - DNA, 7963 bp, synthetic construct
  source 1..7963: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggggtaccgggccccccctcgaggtcgacggtatcgatggatccgatatcgaattcaagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacagagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatgggcatccagggcggcagcgtgctgttcggcctgctgctggtgctggccgtgttctgccacagcggccacagcctgcagtgctacaactgccccaaccccaccgccgactgcaagaccgccgtgaactgcagcagcgacttcgacgcctgcctgatcaccaaggccggcctgcaggtgtacaacaagtgctggaagttcgagcactgcaacttcaacgacgtgaccaccaggctgagggagaacgagctgacctactactgctgcaagaaggacctgtgcaacttcaacgagcagctggagaactaagttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgctggtaccgggccccccctcgaggtcgacggtatcgatggatccgatatcgaattcaacctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacggttactcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcc

SEQ ID NO: 129 - DNA, 7954 bp, synthetic construct
  source 1..7954: mol_type = other DNA
cattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctaggggtaccgggccccccctcgaggtcgacggtatcgatggatccgatatcgaattcaagcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacagagatctcgtttagtgaaccgtcagatcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaagtttaaacgccgccaccatggagttcggcctgagctggctgttcctggtggccatccttaagggcgtgcagtgcgaggtgcagctgcagtgctacaactgccccaaccccaccgccgactgcaagaccgccgtgaactgcagcagcgacttcgacgcctgcctgatcaccaaggccggcctgcaggtgtacaacaagtgctggaagttcgagcactgcaacttcaacgacgtgaccaccaggctgagggagaacgagctgacctactactgctgcaagaaggacctgtgcaacttcaacgagcagctggagaactaagttaactcgagaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagttacctaggaaaactgtcttcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgctggtaccgggccccccctcgaggtcgacggtatcgatggatccgatatcgaattcaacctgcaggcatgctctgagacaataaccctgataaatgcttcaataatgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacgtggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacggttactcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcc

SEQ ID NO: 130-160 - skipped (31 entries)

SEQ ID NO: 161 - protein, 10 aa, synthetic construct
LALGETTRPA

SEQ ID NO: 162 - protein, 10 aa, synthetic construct
LALGETTKPA

SEQ ID NO: 163 - protein, 8 aa, synthetic construct
ALGETTKP

SEQ ID NO: 164 - protein, 8 aa, synthetic construct
ALGETTKP

SEQ ID NO: 165 - protein, 10 aa, synthetic construct
LALGETTKPA

SEQ ID NO: 166 - protein, 10 aa, synthetic construct
LKLGQTTKPK

SEQ ID NO: 167 - protein, 10 aa, synthetic construct
LALGQTTKPK

SEQ ID NO: 168 - protein, 10 aa, synthetic construct
LKLGQTTKPA

SEQ ID NO: 169 - protein, 10 aa, synthetic construct
LALGQTTKPA

SEQ ID NO: 170 - protein, 10 aa, synthetic construct
LALGQTTKPA

SEQ ID NO: 171 - protein, 10 aa, synthetic construct
LALGQTTEPA

SEQ ID NO: 172 - protein, 10 aa, synthetic construct
LALGQTTKPA

SEQ ID NO: 173 - protein, 10 aa, synthetic construct
LALGQTTKPA

SEQ ID NO: 174 - protein, 10 aa, synthetic construct
VALGQTTKPA

SEQ ID NO: 175 - protein, 10 aa, synthetic construct
LALGESTARG

SEQ ID NO: 176 - protein, 10 aa, synthetic construct
LALGETSKRA

SEQ ID NO: 177 - protein, 10 aa, synthetic construct
LALGQSTKPA

SEQ ID NO: 178 - protein, 10 aa, synthetic construct
LALGQTTKPA

SEQ ID NO: 179 - protein, 10 aa, synthetic construct
LALGQTTKPA

SEQ ID NO: 180 - protein, 10 aa, synthetic construct
LALGQTTKPA

SEQ ID NO: 181 - protein, 20 aa, synthetic construct
LALGQTTKPALALGQTTKPA

SEQ ID NO: 182 - protein, 10 aa, synthetic construct
VKLGQTTKPA

SEQ ID NO: 183-190 - skipped (8 entries)

SEQ ID NO: 191 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGETTRPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 192 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 193 - protein, 743 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSALGETTKPGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 194 - protein, 753 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSALGETTKPGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGETTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 195 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLKLGQTTKPKRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 196 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPKRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 197 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLKLGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 198 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 199 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGQTTKPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 200 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTEPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 201 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRLALGQTTKPAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 202 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRLALGQTTKPAGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 203 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNVALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 204 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGESTARGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 205 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGETSKRANRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 206 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGLALGQSTKPANRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 207 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSLALGQTTKPAGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 208 - protein, 755 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGLALGQTTKPATTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 209 - protein, 755 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLALGQTTKPALALGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 210 - protein, 745 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNVAKGQTTKPARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 211-220 - skipped (10 entries)

SEQ ID NO: 221 - protein, 735 aa, Adeno-associated virus 2 [sequence omitted]

SEQ ID NO: 222 - protein, 734 aa, Adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 223 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 224 - protein, 736 aa, Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 225 - protein, 733 aa, Adeno-associated virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 226 - protein, 742 aa, Adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 227-231 - skipped (5 entries)

SEQ ID NO: 232 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
LALGXXXX

SEQ ID NO: 233 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
LKLGXXXX

SEQ ID NO: 234 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
VKLGXXXX

SEQ ID NO: 235 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
VKLGXXXX

SEQ ID NO: 236 - protein, 8 aa, synthetic construct
  VARIANT 5: note = E, S, or Q
  VARIANT 6: note = A, N, Q, S, T, E, D, K, R, or H
  VARIANT 8: note = K, R, E, or A
VKLGXXTX

SEQ ID NO: 237 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
VKLGXXXK

SEQ ID NO: 238 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
LALGXXXX

SEQ ID NO: 239 - protein, 8 aa, synthetic construct
  VARIANT 5: note = E, S, or Q
  VARIANT 6: note = A, N, Q, S, T, E, D, K, R, or H
  VARIANT 8: note = K, R, E, or A
LALGXXTX

SEQ ID NO: 240 - protein, 8 aa, synthetic construct
  VARIANT 5: note = E, S, or Q
  VARIANT 6: note = A, N, Q, S, T, E, D, K, R, or H
  VARIANT 8: note = K, R, E, or A
LALGXXSX

SEQ ID NO: 241 - protein, 8 aa, synthetic construct
  VARIANT 5: note = E, S, or Q
  VARIANT 6: note = A, N, Q, S, T, E, D, K, R, or H
LALGXXTR

SEQ ID NO: 242 - protein, 8 aa, synthetic construct
  VARIANT 5: note = E, S, or Q
  VARIANT 6: note = A, N, Q, S, T, E, D, K, R, or H
LALGXXTK

SEQ ID NO: 243 - protein, 8 aa, synthetic construct
  VARIANT 5: note = E, S, or Q
  VARIANT 6: note = A, N, Q, S, T, E, D, K, R, or H
LALGXXTE

SEQ ID NO: 244 - protein, 8 aa, synthetic construct
  VARIANT 5: note = E, S, or Q
  VARIANT 6: note = A, N, Q, S, T, E, D, K, R, or H
LALGXXTA

SEQ ID NO: 245 - skipped

SEQ ID NO: 246 - protein, 8 aa, synthetic construct
  VARIANT 5: note = E, S, or Q
  VARIANT 6: note = A, N, Q, S, T, E, D, K, R, or H
LALGXXSK

SEQ ID NO: 247 - protein, 8 aa, synthetic construct
  VARIANT 5: note = E, S, or Q
  VARIANT 6: note = A, N, Q, S, T, E, D, K, R, or H
  VARIANT 7: note = A, N, Q, S, T, E, D, K, R, or H
  VARIANT 8: note = K, R, E, or A
LKLGXXXX

SEQ ID NO: 248 - protein, 8 aa, synthetic construct
  VARIANT 5: note = E, S, or Q
  VARIANT 6: note = A, N, Q, S, T, E, D, K, R, or H
  VARIANT 8: note = K, R, E, or A
LKLGXXTX

SEQ ID NO: 249 - protein, 8 aa, synthetic construct
  VARIANT 5: note = E, S, or Q
  VARIANT 6: note = A, N, Q, S, T, E, D, K, R, or H
LKLGXXTK

SEQ ID NO: 250 - DNA, 1029 bp, synthetic construct
  source 1..1029: mol_type = other DNA
atggagttcggcctgagctggctgttcctggtggccatccttaagggcgtgcagtgcgaggtgcagctgggcatctgcgtgtggcaggactggggcgcccaccgctgcaccaacggcggaggcggaagcggcggaggcggatctggcggaggcggaagcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaa

SEQ ID NO: 251 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 252 - DNA, 927 bp, synthetic construct
  source 1..927: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaa

SEQ ID NO: 253 - DNA, 1029 bp, synthetic construct; identical to SEQ ID NO: 250
  source 1..1029: mol_type = other DNA

SEQ ID NO: 254 - DNA, 1023 bp, synthetic construct
  source 1..1023: mol_type = other DNA
atggagttcggcctgagctggctgttcctggtggccatccttaagggcgtgcagtgcgacaagggcatctgcgtgtggcaggactggggcgcccaccgctgcaccaacggcggaggcggaagcggcggaggcggatctggcggaggcggaagcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttaa

SEQ ID NO: 255 - DNA, 864 bp, synthetic construct
  source 1..864: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggcatctgcgtgtggcaggactggggcgcccaccgctgcaccaactaa

SEQ ID NO: 256 - DNA, 1482 bp, synthetic construct
  source 1..1482: mol_type = other DNA
atggtgagctactgggacaccggcgtgctgctgtgcgccctgctgagctgcctgctgctgaccggcagcagcagcggcagcgacaccggcaggcccttcgtggagatgtactccgagatccccgagatcatccacatgaccgagggcagggagctggtgatcccctgcagggtgacctcccccaacatcaccgtgaccctgaagaagttccccctggacaccctgatccccgacggcaagaggatcatctgggactccaggaagggcttcatcatctccaacgccacctacaaggagatcggcctgctgacctgcgaggccaccgtgaacggccacctgtacaagaccaactacctgacccacaggcagaccaacaccatcatcgacgtggtgctgtccccctcccacggcatcgagctgtccgtgggcgagaagctggtgctgaactgcaccgccaggaccgagctgaacgtgggcatcgacttcaactgggagtacccctcctccaagcaccagcacaagaagctggtgaacagggacctgaagacccagtccggctccgagatgaagaagttcctgtccaccctgaccatcgacggcgtgaccaggtccgaccagggcctgtacacctgcgccgcctcctccggcctgatgaccaagaagaactccaccttcgtgagggtgcacgagaaggacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggacgagctgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaagggcggaggcggaagcggcggaggcggatctggcggaggcggaagcggcggaggcggctctggcatctgcgtgtggcaggactggggcgcccaccgctgcaccaactaa

SEQ ID NO: 257 - DNA, 1470 bp, synthetic construct
  source 1..1470: mol_type = other DNA
atggagttcggcctgagctggctgttcctggtggccatccttaagggcgtgcagtgcgaggtgcagctgggcatctgcgtgtggcaggactggggcgcccaccgctgcaccaacggcggaggcggaagcggcggaggcggatctggcggaggcggaagcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctcacagccacagggacttccagcctgtgctgcacctggtggccctgaacagccctctgagcggaggaatgaggggaatcaggggagccgacttccagtgcttccagcaggccagggccgtgggactggccggaaccttcagggccttcctgagcagcaggctgcaggacctgtacagcatcgtgaggagggccgacagggccgccgtgcccatcgtgaacctgaaggacgagctgctgttccctagctgggaggccctgttcagcggaagcgagggacccctgaagcccggagccaggatcttcagcttcgacggaaaggacgtgctgaggcaccctacctggcctcagaagagcgtgtggcacggaagcgaccctaacggaaggaggctgaccgagagctactgcgagacctggaggaccgaggcccctagcgccaccggccaggccagcagcctgctgggaggaaggctgctgggacagagcgccgccagctgccaccacgcctacatcgtgctgtgcatcgagaacagcttcatgaccgccagcaagtga

SEQ ID NO: 258 - DNA, 1464 bp, synthetic construct
  source 1..1464: mol_type = other DNA
atggagttcggcctgagctggctgttcctggtggccatccttaagggcgtgcagtgcgacaagggcatctgcgtgtggcaggactggggcgcccaccgctgcaccaacggcggaggcggaagcggcggaggcggatctggcggaggcggaagcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctcacagccacagggacttccagcctgtgctgcacctggtggccctgaacagccctctgagcggaggaatgaggggaatcaggggagccgacttccagtgcttccagcaggccagggccgtgggactggccggaaccttcagggccttcctgagcagcaggctgcaggacctgtacagcatcgtgaggagggccgacagggccgccgtgcccatcgtgaacctgaaggacgagctgctgttccctagctgggaggccctgttcagcggaagcgagggacccctgaagcccggagccaggatcttcagcttcgacggaaaggacgtgctgaggcaccctacctggcctcagaagagcgtgtggcacggaagcgaccctaacggaaggaggctgaccgagagctactgcgagacctggaggaccgaggcccctagcgccaccggccaggccagcagcctgctgggaggaaggctgctgggacagagcgccgccagctgccaccacgcctacatcgtgctgtgcatcgagaacagcttcatgaccgccagcaagtga

SEQ ID NO: 259 - DNA, 864 bp, synthetic construct
  source 1..864: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggcatctgcgtgtggcaggactggggcgcccaccgctgcgccaactaa

SEQ ID NO: 260 - DNA, 867 bp, synthetic construct
  source 1..867: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggctacatctgcgtgtggcaggactggggcgcccaccgctgcaccaactaa

SEQ ID NO: 261 - DNA, 867 bp, synthetic construct
  source 1..867: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggctacatctgcgtgtggcaggactggggcgcccaccgctgcgccaactaa

SEQ ID NO: 262 - DNA, 861 bp, synthetic construct
  source 1..861: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggcatctgcgtgtggcaggactggggcgcccaccgctgcacctaa

SEQ ID NO: 263 - DNA, 861 bp, synthetic construct
  source 1..861: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggcatctgcgtgtggcaggactggggcgcccaccgctgcgcctaa

SEQ ID NO: 264 - DNA, 864 bp, synthetic construct
  source 1..864: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggctacatctgcgtgtggcaggactggggcgcccaccgctgcacctaa

SEQ ID NO: 265 - DNA, 864 bp, synthetic construct
  source 1..864: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggctacatctgcgtgtggcaggactggggcgcccaccgctgcgcctaa

SEQ ID NO: 266 - DNA, 864 bp, synthetic construct
  source 1..864: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggcatctgcgtgtggcaggactggggcgcccaccgctgcacccagtaa

SEQ ID NO: 267 - DNA, 864 bp, synthetic construct
  source 1..864: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggcatctgcgtgtggcaggactggggcgcccaccgctgcgcccagtaa

SEQ ID NO: 268 - DNA, 867 bp, synthetic construct
  source 1..867: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggctacatctgcgtgtggcaggactggggcgcccaccgctgcacccagtaa

SEQ ID NO: 269 - DNA, 867 bp, synthetic construct
  source 1..867: mol_type = other DNA
atggagttcggcctgagctggctgttcctggtggccatccttaagggcgtgcagtgcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggctacatctgcgtgtggcaggactggggcgcccaccgctgcgcccagtaa

SEQ ID NO: 270 - DNA, 309 bp, synthetic construct
  source 1..309: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgaactgcagcagcgacttcgacgcctgcctgatcaccaaggccggcctgcaggtgtacaacaagtgctggaagttcgagcactgcaacttcaacgacgtgaccaccaggctgagggagaacgagctgacctactactgctgcaagaaggacctgtgcaacttcaacgagcagctggagaactaa

SEQ ID NO: 271 - DNA, 864 bp, synthetic construct; identical to SEQ ID NO: 266
  source 1..864: mol_type = other DNA

SEQ ID NO: 272 - DNA, 1023 bp, synthetic construct
  source 1..1023: mol_type = other DNA
atggagttcggcctgagctggctgttcctggtggccatccttaagggcgtgcagtgcgacaagggcatctgcgtgtggcaggactggggcgcccaccgctgcacccagggcggaggcggaagcggcggaggcggatctggcggaggcggaagcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctgagcaccccaacgcgcgcaaatacaaaggagccaacaagaagggcttgtccaagggctgcttcggcctcaagctggaccgaatcggctccatgagcggcctgggatgttag

SEQ ID NO: 273 - DNA, 1464 bp, synthetic construct
  source 1..1464: mol_type = other DNA
atggagttcggcctgagctggctgttcctggtggccatccttaagggcgtgcagtgcgacaagggcatctgcgtgtggcaggactggggcgcccaccgctgcacccagggcggaggcggaagcggcggaggcggatctggcggaggcggaagcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctcacagccacagggacttccagcctgtgctgcacctggtggccctgaacagccctctgagcggaggaatgaggggaatcaggggagccgacttccagtgcttccagcaggccagggccgtgggactggccggaaccttcagggccttcctgagcagcaggctgcaggacctgtacagcatcgtgaggagggccgacagggccgccgtgcccatcgtgaacctgaaggacgagctgctgttccctagctgggaggccctgttcagcggaagcgagggacccctgaagcccggagccaggatcttcagcttcgacggaaaggacgtgctgaggcaccctacctggcctcagaagagcgtgtggcacggaagcgaccctaacggaaggaggctgaccgagagctactgcgagacctggaggaccgaggcccctagcgccaccggccaggccagcagcctgctgggaggaaggctgctgggacagagcgccgccagctgccaccacgcctacatcgtgctgtgcatcgagaacagcttcatgaccgccagcaagtga

SEQ ID NO: 274 - DNA, 960 bp, synthetic construct
  source 1..960: mol_type = other DNA
atggagttcggcctgagctggctgttcctggtggccatccttaagggcgtgcagtgcgacaagggcatctgcgtgtggcaggactggggcgcccaccgctgcacccagggcggaggcggaagcggcggaggcggatctggcggaggcggaagcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagggcggaggcggaagcggcggaggcggatctggcggaggcggaagcggcggaggcggctctggcatctgcgtgtggcaggactggggcgcccaccgctgcacccagtaa

SEQ ID NO: 275 - DNA, 855 bp, synthetic construct
  source 1..855: mol_type = other DNA
atggagttcggcctgagctggctgttcctggtggccatccttaagggcgtgcagtgcgacaagggcatctgcgtgtggcaggactggggcgcccaccgctgcacccagggcggaggcggaagcggcggaggcggatctggcggaggcggaagcgacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaagtaa

SEQ ID NO: 276 - DNA, 1464 bp, synthetic construct; identical to SEQ ID NO: 273
  source 1..1464: mol_type = other DNA

SEQ ID NO: 277 - protein, 342 aa, synthetic construct
MEFGLSWLFLVAILKGVQCEVQLGICVWQDWGAHRCTNGGGGSGGGGSGGGGSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEHPNARKYKGANKKGLSKGCFGLKLDRIGSMSGLGC

SEQ ID NO: 278 - protein, 239 aa, synthetic construct; identical to SEQ ID NO: 112

SEQ ID NO: 279 - protein, 308 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEHPNARKYKGANKKGLSKGCFGLKLDRIGSMSGLGC

SEQ ID NO: 280 - protein, 342 aa, synthetic construct; identical to SEQ ID NO: 277

SEQ ID NO: 281 - protein, 340 aa, synthetic construct
MEFGLSWLFLVAILKGVQCDKGICVWQDWGAHRCTNGGGGSGGGGSGGGGSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEHPNARKYKGANKKGLSKGCFGLKLDRIGSMSGLGC

SEQ ID NO: 282 - protein, 287 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGICVWQDWGAHRCTN

SEQ ID NO: 283 - protein, 493 aa, synthetic construct
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGICVWQDWGAHRCTN

SEQ ID NO: 284 - protein, 489 aa, synthetic construct
MEFGLSWLFLVAILKGVQCEVQLGICVWQDWGAHRCTNGGGGSGGGGSGGGGSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 285 - protein, 487 aa, synthetic construct
MEFGLSWLFLVAILKGVQCDKGICVWQDWGAHRCTNGGGGSGGGGSGGGGSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 286 - protein, 287 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGICVWQDWGAHRCAN

SEQ ID NO: 287 - protein, 288 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGYICVWQDWGAHRCTN

SEQ ID NO: 288 - protein, 288 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGYICVWQDWGAHRCAN

SEQ ID NO: 289 - protein, 286 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGICVWQDWGAHRCT

SEQ ID NO: 290 - protein, 286 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGICVWQDWGAHRCA

SEQ ID NO: 291 - protein, 287 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGYICVWQDWGAHRCT

SEQ ID NO: 292 - protein, 287 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGYICVWQDWGAHRCA

SEQ ID NO: 293 - protein, 287 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGICVWQDWGAHRCTQ

SEQ ID NO: 294 - protein, 287 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGICVWQDWGAHRCAQ

SEQ ID NO: 295 - protein, 288 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGYICVWQDWGAHRCTQ

SEQ ID NO: 296 - protein, 288 aa, synthetic construct
MEFGLSWLFLVAILKGVQCDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGYICVWQDWGAHRCAQ

SEQ ID NO: 297 - protein, 102 aa, synthetic construct; identical to SEQ ID NO: 42

SEQ ID NO: 298 - protein, 287 aa, synthetic construct; identical to SEQ ID NO: 293

SEQ ID NO: 299 - protein, 340 aa, synthetic construct
MEFGLSWLFLVAILKGVQCDKGICVWQDWGAHRCTQGGGGSGGGGSGGGGSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEHPNARKYKGANKKGLSKGCFGLKLDRIGSMSGLGC

SEQ ID NO: 300 - protein, 487 aa, synthetic construct
MEFGLSWLFLVAILKGVQCDKGICVWQDWGAHRCTQGGGGSGGGGSGGGGSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASK

SEQ ID NO: 301 - protein, 319 aa, synthetic construct
MEFGLSWLFLVAILKGVQCDKGICVWQDWGAHRCTQGGGGSGGGGSGGGGSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSGICVWQDWGAHRCTQ

SEQ ID NO: 302 - protein, 284 aa, synthetic construct
MEFGLSWLFLVAILKGVQCDKGICVWQDWGAHRCTQGGGGSGGGGSGGGGSDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 303 - protein, 487 aa, synthetic construct; identical to SEQ ID NO: 300

SEQ ID NO: 304 - DNA, 297 bp, synthetic construct
  source 1..297: mol_type = other DNA
atggagttcggcctgagctggctgttcctggtggccatccttaagggcgtgcagtgcgacaagctgcagtgctacaactgccccaaccccaccgccgactgcaagaccgccgtgaactgcagcagcgacttcgacgcctgcctgatcaccaaggccggcctgcaggtgtacaacaagtgctggaagttcgagcactgcaacttcaacgacgtgaccaccaggctgagggagaacgagctgacctactactgctgcaagaaggacctgtgcaacttcaacgagcagctggagaactaa

SEQ ID NO: 305 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgaactgcagcagcgacttcgacgcctgcctgatcaccaaggccggcctgcaggtgtacaacaagtgctggaagttcgagcactgcaacttcaacgacgtgaccaccaggctgagggagaacgagctgacctactactgctgcaagaaggacctgtgcaacttcaacgagcagctggagaaccaccatcaccatcaccattaa

SEQ ID NO: 306 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgcaatgcagcagcgacttcgacgcctgcctgatcaccaaggccggcctgcaggtgtacaacaagtgctggaagttcgagcactgcaacttcaacgacgtgaccaccaggctgagggagaacgagctgacctactactgctgcaagaaggacctgtgcaacttcaacgagcagctggagaaccaccatcaccatcaccattaa

SEQ ID NO: 307 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgaactgcagcagcgacttcgacgcctgcctgatcaccaaggccggcctggaggtgtacaacaagtgctggaagttcgagcactgcaacttcaacgacgtgaccaccaggctgagggagaacgagctgacctactactgctgcaagaaggacctgtgcaacttcaacgagcagctggagaaccaccatcaccatcaccattaa

SEQ ID NO: 308 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgaactgcagcagcgacttcgacgcctgcctgatcaccaaggccggcctgcaggtgtacaaccggtgctggaagttcgagcactgcaacttcaacgacgtgaccaccaggctgagggagaacgagctgacctactactgctgcaagaaggacctgtgcaacttcaacgagcagctggagaaccaccatcaccatcaccattaa

SEQ ID NO: 309 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgaactgcagcagcgacttcgacgcctgcctgatcaccaaggccggcctggaggtgtacaaccggtgctggaagttcgagcactgcaacttcaacgacgtgaccaccaggctgagggagaacgagctgacctactactgctgcaagaaggacctgtgcaacttcaacgagcagctggagaaccaccatcaccatcaccattaa

SEQ ID NO: 310 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgcaatgcagcagcgacttcgacgcctgcctgatcaccaaggccggcctgcaggtgtacaaccggtgctggaagttcgagcactgcaacttcaacgacgtgaccaccaggctgagggagaacgagctgacctactactgctgcaagaaggacctgtgcaacttcaacgagcagctggagaaccaccatcaccatcaccattaa

SEQ ID NO: 311 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgcaatgcagcagcgacttcgacgcctgcctgatcaccaaggccggcctggaggtgtacaaccggtgctggaagttcgagcactgcaacttcaacgacgtgaccaccaggctgagggagaacgagctgacctactactgctgcaagaaggacctgtgcaacttcaacgagcagctggagaaccaccatcaccatcaccattaa

SEQ ID NO: 312 - protein, 98 aa, synthetic construct
MEFGLSWLFLVAILKGVQCDKLQCYNCPNPTADCKTAVNCSSDFDACLITKAGLQVYNKCWKFEHCNFNDVTTRLRENELTYYCCKKDLCNFNEQLEN

SEQ ID NO: 313 - protein, 108 aa, synthetic construct
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENHHHHHH

SEQ ID NO: 314 - protein, 108 aa, synthetic construct
MGIQGGSVLFGLLLVLAVFCHSGHSLQCYNCPNPTADCKTAVQCSSDFDACLITKAGLQVYNKCWKFEHCNFNDVTTRLRENELTYYCCKKDLCNFNEQLENHHHHHH

SEQ ID NO: 315 - protein, 108 aa, synthetic construct
MGIQGGSVLFGLLLVLAVFCHSGHSLQCYNCPNPTADCKTAVNCSSDFDACLITKAGLEVYNKCWKFEHCNFNDVTTRLRENELTYYCCKKDLCNFNEQLENHHHHHH

SEQ ID NO: 316 - protein, 108 aa, synthetic construct
MGIQGGSVLFGLLLVLAVFCHSGHSLQCYNCPNPTADCKTAVNCSSDFDACLITKAGLQVYNRCWKFEHCNFNDVTTRLRENELTYYCCKKDLCNFNEQLENHHHHHH

SEQ ID NO: 317 - protein, 108 aa, synthetic construct
MGIQGGSVLFGLLLVLAVFCHSGHSLQCYNCPNPTADCKTAVNCSSDFDACLITKAGLEVYNRCWKFEHCNFNDVTTRLRENELTYYCCKKDLCNFNEQLENHHHHHH

SEQ ID NO: 318 - protein, 108 aa, synthetic construct
MGIQGGSVLFGLLLVLAVFCHSGHSLQCYNCPNPTADCKTAVQCSSDFDACLITKAGLQVYNRCWKFEHCNFNDVTTRLRENELTYYCCKKDLCNFNEQLENHHHHHH

SEQ ID NO: 319 - protein, 108 aa, synthetic construct
MGIQGGSVLFGLLLVLAVFCHSGHSLQCYNCPNPTADCKTAVQCSSDFDACLITKAGLEVYNRCWKFEHCNFNDVTTRLRENELTYYCCKKDLCNFNEQLENHHHHHH

SEQ ID NO: 320 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgaactgcagcagcgacttcgacgcctgcctgatcaccaaggccggcgagcaggtgtacaacaagtgctggaagttcgagcactgcaacttcaacgacgtgaccaccaggctgagggagaacgagctgacctactactgctgcaagaaggacctgtgcaacttcaacgagcagctggagaaccaccatcaccatcaccattaa

SEQ ID NO: 321 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgaactgcggcagcgacttcgacgcctgcctgatcaccaaggccggcctgcaggtgtacaacaagtgctggaagttcgagcactgcaacttcaacgacgtgaccaccaggctgagggagaacgagctgacctactactgctgcaagaaggacctgtgcaacttcaacgagcagctggagaaccaccatcaccatcaccattaa

SEQ ID NO: 322 - DNA, 332 bp, synthetic construct
  source 1..332: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgaactgcagcagcgacttcgacgcctgcctgatcaccaaggccggcctgcaggtgtacaacaagtgctggaagttcgagcactgcaacttcaacgacgtgggcaccaggctgagggagaacgagctgacctactactgctgcaagaaggacctgtgcaacttcaacgagcagctggagaaccaccatcaccatcaccattaagttaa

SEQ ID NO: 323 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgaactgcggcagcgacttcgacgcctgcctgatcaccaaggccggcctgcaggtgtacaacaagtgctggaagttcgagcactgcaacttcaacgacgtgggcaccaggctgagggagaacgagctgacctactactgctgcaagaaggacctgtgcaacttcaacgagcagctggagaaccaccatcaccatcaccattaa

SEQ ID NO: 324 - DNA, 327 bp, synthetic construct
  source 1..327: mol_type = other DNA
atgggcatccagggcggcagcgtgctgttcggcctgctgctggtgctggccgtgttctgccacagcggccacagcctgcagtgctacaactgccccaaccccaccgccgactgcaagaccgccgtgcaatgcagcagcgacttcgacgcctgcctgatcaccaaggccggcctggaggtgtacaacaagtgctggaagttcgagcactgcaacttcaacgacgtgaccaccaggctgagggagaacgagctgacctactactgctgcaagaaggacctgtgcaacttcaacgagcagctggagaaccaccatcaccatcaccattaa

SEQ ID NO: 325 - protein, 108 aa, synthetic construct
MGIQGGSVLFGLLLVLAVFCHSGHSLQCYNCPNPTADCKTAVNCSSDFDACLITKAGEQVYNKCWKFEHCNFNDVTTRLRENELTYYCCKKDLCNFNEQLENHHHHHH

SEQ ID NO: 326 - protein, 108 aa, synthetic construct
MGIQGGSVLFGLLLVLAVFCHSGHSLQCYNCPNPTADCKTAVNCGSDFDACLITKAGLQVYNKCWKFEHCNFNDVTTRLRENELTYYCCKKDLCNFNEQLENHHHHHH

SEQ ID NO: 327 - protein, 108 aa, synthetic construct
MGIQGGSVLFGLLLVLAVFCHSGHSLQCYNCPNPTADCKTAVNCSSDFDACLITKAGLQVYNKCWKFEHCNFNDVGTRLRENELTYYCCKKDLCNFNEQLENHHHHHH

SEQ ID NO: 328 - protein, 108 aa, synthetic construct
MGIQGGSVLFGLLLVLAVFCHSGHSLQCYNCPNPTADCKTAVNCGSDFDACLITKAGLQVYNKCWKFEHCNFNDVGTRLRENELTYYCCKKDLCNFNEQLENHHHHHH

SEQ ID NO: 329 - protein, 108 aa, synthetic construct
MGIQGGSVLFGLLLVLAVFCHSGHSLQCYNCPNPTADCKTAVQCSSDFDACLITKAGLEVYNKCWKFEHCNFNDVTTRLRENELTYYCCKKDLCNFNEQLENHHHHHH

SEQ ID NO: 330 - protein, 8 aa, synthetic construct
GGGSGGGS

SEQ ID NO: 331 - protein, 4 aa, synthetic construct
EVQL

SEQ ID NO: 332 - protein, 26 aa, unidentified
GGTSLSEKTVLLLVTPFLAAAWSLHP

SEQ ID NO: 333 - protein, 6 aa, synthetic construct
HHHHHH

SEQ ID NO: 334 - protein, 19 aa, synthetic construct
EVQLGICVWQDWGAHRCTN

SEQ ID NO: 335 - protein, 17 aa, synthetic construct
DKGICVWQDWGAHRCTN

SEQ ID NO: 336 - protein, 16 aa, synthetic construct
GGGSGGGSGGGSGGGS

SEQ ID NO: 337 - protein, 15 aa, unidentified
GICVWQDWGAHRCTN